(19) United States
(12) Patent Application Publication
(10) Pub. No.: US 2012/0202814 A1
(43) Pub. Date: Aug. 9, 2012

(54) COMPOUNDS WITH DDX3 INHIBITORY ACTIVITY AND USES THEREOF

(75) Inventors: Marco Radi, Siena (IT); Maurizio Botta, Siena (IT); Federico Falchi, Siena (IT); Giovanni Maga, Pavia (IT); Fausto Baldanti, Borgonovo V.T. (PC) (IT); Stefania Paolucci, Borgarello (PV) (IT)

(73) Assignees: UNIVERSITÀ DEGLI STUDI DI SIENA, Siena (IT); CONSIGLIO NAZIONALE DELLE RICERCHE, Roma (IT)

(21) Appl. No.: 13/499,547

(22) PCT Filed: Oct. 4, 2010
(86) PCT No.: PCT/IB10/54475

§ 371 Date: Apr. 19, 2012

Related U.S. Application Data
(60) Provisional application No. 61/248,030, filed on Oct. 2, 2009.

(51) Int. Cl. C07D 413/04; A61K 31/426; A61K 31/53; C07D 251/54; A61P 31/12; A61K 31/535; A61K 31/505; C07D 239/48; C12N 9/99; C12N 5/00; C07D 277/36; C07D 403/12

(52) U.S. Cl. 514/236.2; 548/183; 514/369; 514/245; 544/197; 544/198; 544/113; 514/275; 544/323; 435/184; 435/375

(57) ABSTRACT

The present invention relates to the medical use of the compound of formula 1, 2, 3 or 4:

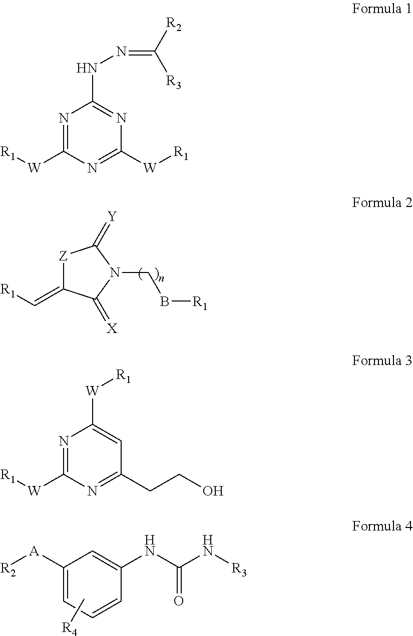

### FIELD OF THE INVENTION

The present invention refers to compounds with cellular RNA helicase and/or ATPase DDX3 inhibitory activity and their therapeutic use, in particular for the treatment of viral and neoplastic diseases. The invention relates also to a method for identifying compounds endowed with binding capacities to the target sites on DDX3.

### BACKGROUND OF THE INVENTION

### DDX3 and HIV-1 Infections

### Cellular Cofactors as Potential Novel Targets for Suppressing HIV-1 Replication.

All compounds that are currently approved for the treatment of HIV infections target viral proteins: The enzymes reverse transcriptase (RT), protease (PR), integrase (IN) and, introduced more recently, the fusion peptide gp41 of the viral Env, which is essential for HIV entry. In considering how to design antiviral drugs, one could envision two different strategies. In fact, since viruses, including HIV, have evolved the capacity to exploit the cell's molecular machineries as essential components of their replicative cycle, agents designed to interrupt viral replication could, in principle, target with equal effectiveness either a viral or cellular polypeptide. These two strategies hold different implications. In the case of a unique viral function, such as retrotranscription or virion assembly, there is a lower risk of creating inhibitors with toxic effects on the host. However, the Achilles' heel of such an approach is viral resistance to the drugs. Unless a drug is incredibly potent (reducing the size of the replicating pool of virus rapidly), and therefore requires only a short duration of treatment (reducing the time for the resistant viruses to amplify), resistance to treatment will arise over time, as observed with HIV or HBV patients on therapy. The alternative approach, targeting a cellular factor that is required for viral replication should help to overcome the problem of viral resistance, but it has a major potential of causing greater toxicity to the host. Theoretically, a drug targeting a cellular factor could inhibit all viruses that are dependent on the same host factor. Thus, it has become a paradigm in antiviral research, that the understanding of the dynamic interplay of host cell and virus is essential to develop effective strategies for controlling viral infection. This holds true also for HIV. It has been shown that several cellular proteins are employed as essential co-factors by HIV. Established co-factors for HIV replication are the chemokine receptors, CXCR4 and CCR5, that, in conjunction with CD4, are essential for viral entry into target cells. But also after entry, cellular proteins play essential roles within the viral replication cycle. Cellular binding partners exist for example for HIV-1 integrase such as integrase interactor 1 (Inil) which has amino acid similarity to the yeast transcriptional activator SNF5, a component of the multiprotein SWI/SNF complex (Kalpana et al., 1994), and HMGA1, a non-histone chromosomal protein important for transcriptional control and chromosomal architecture (Farnet and Bushman, 1997). The Debyser group has recently identified Lens Epithelium Derived Growth Factor (LEDGF) as a novel binding partner of HIV-1 integrase (Cherepanov et al., 2003), using Fluorescence Cross Correlation Spectroscopy as an innovative technology to study protein-protein interactions in the living cell (Maertens et al., 2005). Besides integration, other steps of the replicative cycle of HIV-1 require the essential contribution of specific cellular proteins, including viral transcription (cellular Cyclin T1 (Wei et al., 1998)) and viral assembly and egress (e.g. TSG101, a member of the cellular ESCRT machinery (von Schwedler et al., 2003)). From another perspective, the critical role of cellular co-factors gains strong support from the finding that HIV-1 is unable to replicate in non-human cells, including cells from non-human primates, mice, and rats (Cohen, 2001). Apparently, HIV-1 replication is subject to potent restrictions in these species. Over the years, important advances have been made in elucidating the molecular basis of such blocks to HIV-1 replication, with a particular emphasis on rodent cells. In fact, the critical role of several cellular co-factors for HIV-1 infection, including the CXCR4 co-receptor and the transcription enhancing factor Cyclin T1, which is a component of the pTEFb transcription factor complex (Wei et al., 1998), was first identified in mouse cells. Expression of the human protein, but not of the mouse ortholog, allowed the HIV replication cycle to progress. Remarkably, a single amino acid difference between mouse and human Cyclin T1 appears to determine its ability to interact with the viral Tat protein. Subsequent inhibition or knock-down studies in human cells confirmed the pivotal role of CXCR4 and Cyclin T1 for HIV-1 replication. Thus, the characterization of intrinsic restrictions in the HIV-1 replication cycle in rodent cells is a powerful approach for the identification of essential cellular co-factors and potential drug targets in human cells. Furthermore, pinpointing such species-specific restrictions provides the basis to overcome these blocks and advance the development of a fully-permissive transgenic small animal model of HIV-1 infection (Cohen, 2001). Here, the Keppler group previously demonstrated that immuno-competent rats, that transgenically express the human CD4/CCR5 receptor complex, support a robust cellular infection and transient, low-level viremia (Keppler et al., 2005; Keppler et al., 2002; Keppler et al., 2001). In particular primary macrophages from transgenic rats supported the full HIV-1 replication cycle and secreted infectious virions.

### The Essential Rev-Dependent HIV RNA Nuclear Export Pathway and the Role of the RNA Helicase DDX3.

The nuclear export of viral RNAs is a crucial step in HIV-1 replication, since the appropriate expression of viral genes paves the way to all the following steps like encapsidation, virion assembly and budding. Any drug targeting this step will act at an earlier level than protease inhibitors, shutting down viral proliferation at a step where no structural viral proteins (Gag-Pol, Env) or even full length genomic RNA have yet been produced in the infected cells. Due to its outmost importance, it is of no surprise that the export of partially spliced or unspliced viral transcripts is one of the most complex and tightly regulated steps of the entire HIV-1 life cycle. It has to be stressed that normal, uninfected cells have evolved strict quality control pathways to avoid the nuclear export of partially spliced transcripts, in order to ensure that no aberrant proteins will be produced upon translation by ribosomes (Cullen, 2003). Thus, in order to allow nuclear export of its own unprocessed or partially processed transcripts, HIV-1 has to completely subvert and take control of these cellular machineries, and this is why at this particular step of the viral life cycle, functional interactions between viral and cellular proteins do likely play a pivotal role.

### The Rev/RRE-Dependent HIV RNA Export Pathway.

All nucleo-cytoplasmic transport processes take place through the nuclear pore complex (NPC), a large dynamic multi-protein assembly that acts as the passageway for transport (Pante, 2004). This active transport can also occur against a concentration gradient, and is mediated by soluble transport factors, that in turn shuttle between the nucleus and the cytoplasm. Importantly, even molecules that are theoretically small enough for passive diffusion (e.g., HIV-1 Rev) are actively and selectively transported, since regulated transport appears to be more efficient and more amendable for specific regulation (Pemberton and Paschal, 2005). HIV-1 has a total of nine genes that are expressed by alternative splicing of a single, initial proviral transcript that also forms the RNA genome. Importantly, HIV-1 replication requires the nuclear export and translation of unspliced, singly-spliced and multiply-spliced derivatives of this initial transcript. Fully spliced mRNAs encode the viral regulatory proteins Tat, Rev and Nef, whereas incompletely spliced HIV-1 mRNAs primarily encode viral auxiliary (Vif, Vpr, Vpu) and structural proteins. The HIV-1 Rev is a sequence-specific nuclear mRNA-export factor. In the absence of Rev function, the incompletely spliced HIV-1 mRNAs that encode the viral structural proteins are retained in the cell nucleus, whereas nuclear export of fully-spliced HIV-1 mRNAs, including the mRNA encoding Rev itself, is independent of Rev function. Nuclear export of unspliced HIV-1 mRNAs also requires a structured cis-acting RNA sequence, called the Rev response element (RRE), which is specifically bound by Rev. The nuclear retention of the incompletely spliced viral mRNAs in the absence of Rev results from the fact that splice sites present in the retained introns are recognized by cellular mRNA processing factors, termed splicing commitment factors, that normally prevent cellular pre-mRNAs (i.e. incompletely spliced cellular mRNAs) from exiting the nucleus. (Cullen, 2003). The HIV-1 Rev protein has two distinct functional domains, an N-terminal sequence required for RRE binding and Rev multimerization and a 10-amino-acid leucine-rich domain near the C-terminus that serves as the Rev nuclear export signal (NES). This signal mediates the nuclear export of the Rev/RNA complex, through interaction with the cellular export receptor Crm1 and proposed additional cellular cofactors (Yedavalli et al., 2004).

### The Crm1-Dependent Cellular RNA Export Pathway.

Crm1 is a member of the karyopherin-beta family of nucleocytoplasmic-transport factors. Karyopherin-beta members associate with karyopherin-alpha in the cytoplasm, forming a heterodimeric complex with proteins containing a nuclear localisation signal (NLS). Then, karyopherin-beta interacts with the nuclear pore complex (NPC), facilitating the import of the karyopherin-alpha/NLS-protein complex. Recently, however, members of the karyopherin-beta family have been identified which regulate nuclear export, rather than import, pathways. One of such proteins is Crm1, which shares homology with karyopherin beta1, beta2, beta3 and beta4. Human Crm1 is localized both at the NPC and in the nucleoplasm and seems to shuttle between the nucleus and cytoplasm. It is now clear that Crm1 is the crucial nuclear export factor for two classes of cellular RNAs, that is, U-rich small nuclear RNAs and ribosomal RNAs. Crm1 binds its cargo in the nucleus in the presence of the GTP-bound form of the Ran GTPase. After nuclear export, hydrolysis of the bound GTP to GDP causes a conformational shift that induces cytoplasmic cargo release, thus providing the directionality of this export pathway. Crm1 also interacts with components of the NPC, the portal used for all nucleocytoplasmic transport, and this interaction is essential for Crm1-mediated nuclear RNA export.

### The DDX3 Family of Cellular RNA Helicases.

Recently, the DEAD-box RNA helicase DDX3 was identified as an essential cofactor for Rev/RRE-mediated HIV RNA-export (Yedavalli et al., 2004). RNA helicases from the DEAD-box family are found in almost all organisms and have important roles in RNA metabolism. They are associated with many processes ranging from RNA synthesis to RNA degradation. DEAD-box proteins use the energy from ATP hydrolysis to rearrange inter- or intra-molecular RNA structures or dissociate RNA-protein complexes. Human DEAD-box family includes 36 members. The majority of DEAD-box family members have demonstrated functions in ribosome biogenesis and translation initiation. In addition to a function in RNA metabolism, two other functional features appear to be present in this gene family. The first feature is dysregulation in cancer, which occurs in the form of involvement in recurrent chromosomal translocations (DDX6, DDX10) or overexpression (DDX1, DDX6, DDX4). The second feature is the involvement of DDX members in tissue-organ differentiation (DDX4 in germ cell development, DDX5 in organ differentiation, DDX25 in spermatogenesis, and DDX41 in visual system development). Therefore, putative RNA helicases may have a function in differentiation, possibly by their effect on the expression of critical differentiation genes.

### The Human Cellular RNA Helicase DDX3/HIV-1 Rev Axis.

DDX3 expression was found to be induced in HIV-1 infected cells by the viral transcriptional activator Tat, but it apparently plays no role in HIV-1 transcription. Instead, DDX3 was shown to be an RNA-dependent ATPase/helicase which functions in the Rev-RRE/CRM1 pathway for the export of unspliced/partially spliced HIV-1 transcripts. DDX3 is a nucleo-cytoplasmic shuttling protein that binds Crm1 and associates to the cytoplasmic side of nuclear pores. Its enzymatic activity, as well as its physical interaction with Crm1 is necessary for Rev/RRE mediated nuclear export of viral RNAs. However, the molecular details of its role(s) in this pathway are yet to be elucidated.

### DDX3 and HBV/HCV Infections

A significant downregulation of DDX3 expression is found in hepatocellular carcinoma (HCCs) from hepatitis B virus (HBV)-positive patients, but not from HCV-positive ones, compared to the corresponding nontumor tissues. The expression of DDX3 is differentially regulated by the gender and, moreover, there is a tendency that the downregulation of DDX3 expression in HCCs is more frequent in males than in females. Genetic knockdown of DDX3 with small interfering RNAs (siRNA) in a nontransformed mouse fibroblast cell line, NIH-3T3, results in a premature entry to S phase and an enhancement of cell growth. This enhanced cell cycle progression is linked to the upregulation of cyclin D1 and the downregulation of p21 (WAF1) in the DDX3 knockdown cells. In addition, constitutive reduction of DDX3 expression increases the resistance of NIH-3T3 cells to serum depletion-induced apoptosis and enhances the ras-induced anchorage-independent growth, indicating the involvement of DDX3 in cell growth control. These suggest that the deregulation of DDX3, a DEAD box RNA helicase with cell growth-regulatory functions, is involved in HBV- and HCV-associated pathogenesis. Owsianka and Patel (1999) have shown that DDX3 interact specifically with hepatitis C virus (HCV) core protein and thus resulted in a change of its intracellular location. Huang et al. (2004) have demonstrated that the mRNA expression of DDX3 is up-regulated in hepatocellular carcinoma (HCC). Since many lines of evidence indicate that HCV core protein is the oncogenic agent that activates the neoplastic transformation pathways in HCC, the overexpression of DDX3 in HCC tissue could play a role in the downstream transformation pathway or in synergy to the transformation capability of HCV core protein in HCC.

### DDX3 and Cancers

Chao et al. (2006) showed that the subcellular localization of DDX3 is alterated in cutaneous squamous cell carcinoma (SCC).

In 2008 Botlagunta et al. (2008) using immortalized breast cell lines and breast cancer cells, showed that DDX3 expression correlates with an aggressive tumoral phenotype. The human breast cell line, MCF 10A, was characterized for the gene expression pattern. Of the differential genes expressed, it was found consistent activation of DDX3, a member of the DEAD box RNA helicase family. Overexpression of DDX3 in MCF 10A cells induced an epithelial-mesenchymal-like transformation, exhibited increased motility and invasive properties, and formed colonies in soft-agar assays. Besides the altered phenotype, MCF 10A-DDX3 cells repressed E-cadherin expression as demonstrated by both immunoblots and by E-cadherin promoter-reporter assays. In addition, an in vivo association of DDX3 and the E-cadherin promoter was demonstrated by chromatin immunoprecipitation assays. Collectively, these results demonstrate that the activation of DDX3 by BPDE, can promote growth, proliferation and neoplastic transformation of breast epithelial cells. It is possible that the regulatory mechanisms of DDX3 in various cell types are different depending upon the presence of appropriate cofactor and/or signaling pathways

As indicated above, current pharmacological therapies against HIV-1 infections are based on multiple drug combinations. Most used and cornerstone drugs act upon viral enzymes: reverse transcriptase (RT), protease (PR), integrase (IN). In addition, one class of drugs inhibits HIV entry in cells by competing for the recepetor/coreceptor usage. While drugs directed against RT and PR are widely used, their efficacy is hampered by mutations in the target enzymes, leading invariably to drug resistance and chemotherapy failure. The protein DDX3 has been shown to 1) be an essential cofactor for the replication of the HIV-1 and HCV viruses; 2) be involved in the proliferation of cancer cells. The DDX3-inhibitors are therefore considered as potential agents not only in the treatment of HIV-1 infections, but also for other viruses such as HCV, as well as in the treatment of various kinds of cancer, such as hepatocellular carcinoma.

The combination of DDX3-inhibitors to the existing antiviral and anticancer therapy would also improve the outcome of the treatment.

No inhibitors of DEAD-box RNA helicases DDX3 have been described so-far. A compound has been recently published as a DDX3 inhibitor (Yedavalli, V. S. R. K. et al. J. Med. Chem. 2008, 51, 5043-5051). However, direct inhibition of RNA helicases has not been proven.

Maga et al. (J. Med. Chem. 2008, 51, 6635-6638) disclose a receptor-based Pharmacophore Models of the ATP-binding site to be used for the identification of DDX3 inhibitors. The three dimensional arrangements of the functional groups of an inhibitor described by the Pharmacophoric Models (essential for the effective binding to DDX3) has been used for a three-dimensional (3D) database search.

The present invention describes compounds with cellular DDX3 inhibitory activity and their use in the treatment of viral and neoplastic diseases. A method for the identification of such inhibitors, based on an homology model of the closed conformation of DDX3 to locate the RNA-binding site and Virtual Docking protocol is disclosed. The synthesis of the compounds is also provided.

The invention illustrate a novel approach to treat HIV/AIDS, namely targeting a cellular enzyme (the DEAD-box RNA helicases DDX3) which has been recognized as a co-factor for HIV replication, thus rendering the infected cell an unfavorable environment for viral replication. The advantage of such approach is ideally represented by the possibility to overcome the drug resistance problem connected to common anti-HIV drugs, since cellular enzymes do not have the high mutation rates typical of viral enzymes.

### SUMMARY OF THE INVENTION

The present invention describes compounds able to suppress the enzymatic functions of the cellular protein DDX3 (Dead-box polypeptide 3; Ref. Seq. NP 001347), namely: ATP hydrolysis (ATPase) and/or DNA/RNA unwinding (helicase).

The compounds presented in this invention (Formula 1-4) surprisingly showed:

1) the ability to selectively suppress the enzymatic activity of DDX3 in vitro;
2) the ability to suppress HIV-1 replication in infected cells;
3) the ability to suppress proliferation of tumor cell lines.

These experimental findings make the new compounds useful as both antiviral and anticancer therapy.

It is therefore an object of the invention a compound of formula 1, 2, 3 or 4 for use as a treatment of a pathology modulated by DDX3 activity and/or expression:

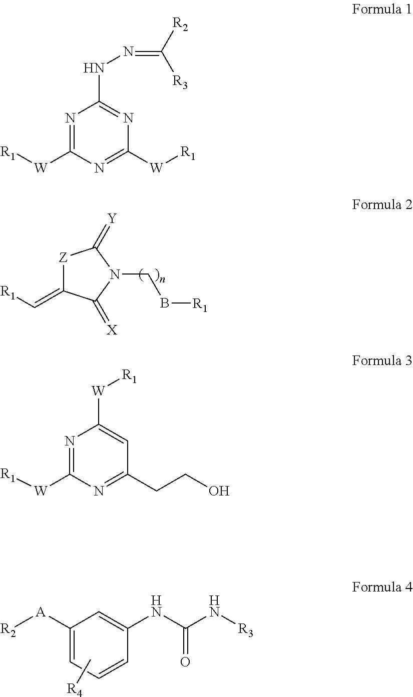

wherein:
Z represents CH₂ or S;
X and Y represent independently O or S;
n is comprised between 0 and 4;
B is absent or represents

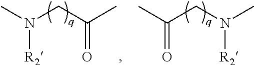

q is comprised between 0 and 2 and R₂′ represents H, —(CH₂)_{w′}—OH, —(CH₂)_{w′}—NH₂, w′ is an integer from 1 to 3; B is also C═O;
R₁, R₂, R₃ are each independently selected from the group of: H, a linear or branched alkyl group comprising 1 to 6 carbon atoms, unsubstituted or substituted phenyl radical, an unsubstituted or substituted phenylalkenyl radical, an unsubstituted or substituted phenyalkynyl radical, an unsubstituted or substituted biphenylalkyl radical, an unsubstituted or substituted heterocyclic radical, an unsubstituted or substituted polycyclic radical, an unsubstituted or substituted alicyclic radical or a radical of the formula (R₁a-)ₘ(L-)ₚR₁b-, wherein R₁a and R₁b may be the same or different and represent an unsubstituted or substituted heterocyclic radical or an unsubstituted or substituted phenyl radical, R₁a also represents an unsubstituted or substituted polycyclic radical and L represents a divalent linking moiety selected from the group consisting of a valence bond, —(CH₂)_{q}—, —HC═CH—, —C≡C—, —C(═O)—, —O—, —S—, —S(═O)—, —S(═O)₂—, —NHCONH— or NR₁c, R₁c being hydrogen or alkyl, m and p are each independently 0 or 1, and q is an integer from 1 to 3;
R₂ and R₃ together optionally form a cycloalkyl, cycloalkenyl, non-aromatic heterocyclic, or fused or polycyclic ring, 2-oxindole wherein said cycloalkyl, cycloalkenyl, non-aromatic heterocyclic and fused or polycyclic ring are optionally substituted with one or more substituents selected from the groups above described. Examples of suitable fused or polycyclic ring are substituted or unsubstituted 2-oxindoles; substituted or unsubstituted 1H-indene-1,3-diones
wherein the heterocyclic radical is selected from the group consisting of morpholine, thiomorpholine, piperidine, piperazine, pyrrolidine, furan, thiophene, oxazole, oxadiazole, isoxazole, pyridine, 1,3-oxathio lane, thiazole, isothiazole, thiadiazole, imidazole, pyrrole, tetrazole or triazine;
wherein the polycyclic radical is selected from the group consisting benzofuran, isobenzofuran, benzothiophene, isobenzothiophene, benzoxazole, indole, 2-isoindole, 2-oxindole, 2-methylindole, benzopyrazole, quinoline, isoquinoline, tetrahydroquinoline, 1,3-benzodioxole, 1,2-benzodiazine, 1,3-benzodiazine, 1,2,3-benzotriazole, benzothiazole, benzimidazole, 1,2,3-benzotriazine, 1,2,4-benzotriazine, naphtalene, antracene or fluorene; wherein the alicyclic radical preferably comprises 5 to 8 carbon atoms. Examples of suitable cycloalkyls are cyclopentyl, cyclohexyl, methylcyclohexyl and norbornyl.
wherein the heterocyclic radical substituents, the polycyclic radical substituents and the alicyclic radical substituents being at least one selected from the group consisting of straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;
wherein the phenyl radical substituents, the phenylalkenyl radical substituents, the phenylalkynyl radical substituents or the biphenylalkyl radical substituents are selected from the group consisting of a straight or branched chain, saturated or unsaturated aliphatic group having 1-6 carbon atoms, halogen, nitro, carboxy, carboxy alkyl, alkoxy, hydroxy, hydroxyalkyl, perhaloalkoxy, acyl, acyloxy, acyloxyalkyl, cyano, carbalkoxy, thio, alkylhio, alkylsulfinyl, alkylsulfonyl, amino, alkylamino, dialkylamino, aminoalkyl, alkylaminoalkyl, dialkylaminoalkyl, sulfonamido, carboxamido, alkanoylamino;
W is absent or represents independently O, S, NH, NHCH₂ or N—R₅ wherein R₅ is a linear or branched alkyl group comprising 1 to 6 carbon atoms;
A is absent or represents CONH, NHCO, NHCONH;
R₄ represents H, unsubstituted or substituted alkyl from 1 to 6 carbon atoms, unsubstituted or substituted alkenyl, unsubstituted or substituted alkynyl, halogen, haloalkyl, COOH, OCH₃, NO₂, NH₂, CN, OZ′ or SZ′ where Z′ is H, or unsubstituted or substituted alkyl from 1 to 6 carbon atoms;
with the provisio that the compounds indicated below are not comprised

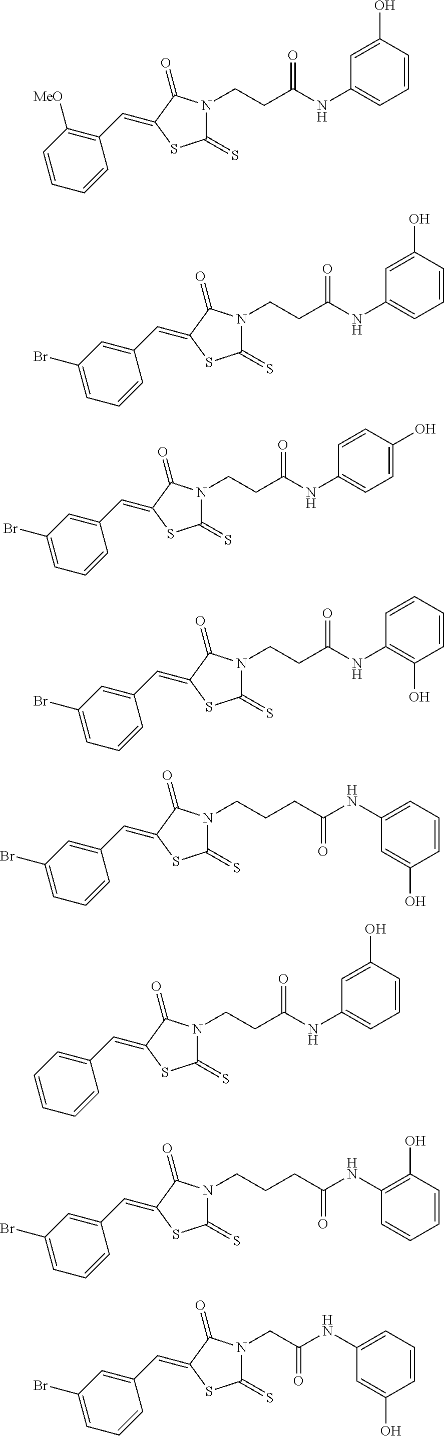

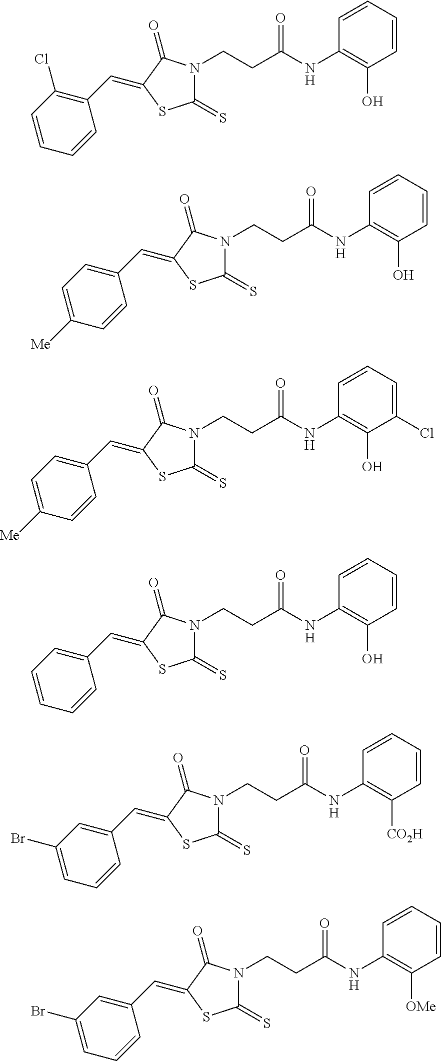

Preferably, the compound has formula 1 or 3 wherein W is absent or independently selected from NH, NHCH₂ or N—R₅ wherein R₅ ethyl; R₁ is independently selected from the group of ethyl, heterocyclic radical, unsubstituted or substituted phenyl radical and polycyclic radical; R₂ is selected from the group of unsubstituted or substituted phenyl radical, heterocyclic radical or polycyclic radical when R₃═H or Me; alternatively, R₂ and

R₃ together form a 2-oxindole.

Still preferably, the compound is the compound 11d, 14f, 14g, 15h, 15k, 38, FE56, FE56M, FE56F, FE56N, FE56AN or 22.

In a preferred embodiment, the compound has formula 2 wherein Z=S; Y=S; X=O; n=2;

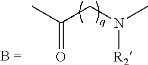

or C═O; q=0; R₂′=H; R₁ is independently selected from the group of substituted phenyl radical or polycyclic radical

Still preferably, the compound is the compound 35b, 35c, 35f, FE-70, FE-77, FE-69, FE-74.

Preferably, the compound has formula 4 wherein R₂, R₃ are independently selected from the group of H, unsubstituted or substituted phenyl radical; R₄═H, unsubstituted or substituted alkyl from 1 to 6 carbon atoms, COOH, OCH₃, NO₂, NH₂; A is absent or represents CONH, NHCO, NHCONH.

Still preferably, the compound is the compound EI-01, EI-01A, EI-01B, 25, 26, 27, 29, 30, 31.

Preferably the compound is an inhibitor of the human DEAD-box RNA helicases DDX3.

Still preferably the compound is an inhibitor of ATPase and/or helicase of the human DEAD-box RNA helicases DDX3.

In a preferred embodiment the pathology modulated by DDX3 activity and/or expression is an infection or a hyperproliferative disease. Preferably, the infection is a viral infection. Still preferably, the viral infection is caused by HIV, HBV, HCV or poxviruses. More preferably it is caused by HIV-1.

In a preferred embodiment the hyperproliferative disease is a tumor. Preferably, the tumour is selected from the group of: hepatocellular carcinoma, cervical cancer, breast cancer, lymphomas, leukemias.

It is a further object of the invention a pharmaceutical composition comprising the compound as defined above, or a pharmaceutically acceptable salt, solvate, or hydrate thereof and pharmaceutically acceptable excipients for use as a treatment of a pathology modulated by DDX3 activity and/or expression.

In the present invention a pathology modulated by DDX3 activity and/or expression is defined as a pathology that can be induced, triggered or enhanced by DDX3 protein. The activity of DDX3 can be measured by techniques known in the art for instance by measuring the enzymatic activity of the protein (ATPase or helicase activity). The expression of DDX3 is also measured by commonly used methods in the art. The compounds of the present invention are particularly suitable for the treatment of patient that are resistant to at least one currently used treatment for HIV infection. For instance, patient resistant to RT inhibitors, PR inhibitors and/or IN inhibitors.

In the present invention the compounds as defined above and suitable excipients and/or diluents may be administered in combination with pharmaceutical compositions of approved drugs for the treatment of the HIV-1 infections as part of highly active antiretroviral therapy (HAART).

The pharmaceutical composition of the invention may comprise a combination of at least two of the compounds of the invention or a pharmaceutically acceptable salt thereof, and suitable excipients and/or diluents and may be also administered in combination with pharmaceutical compositions of approved drugs for the treatment of the HCV infections as part of combinatorial multidrug anti-HCV therapy.

The pharmaceutical composition of the invention may comprise a combination of at least two of the compounds of the invention or a pharmaceutically acceptable salt thereof, and suitable excipients and/or diluents and may be also administered in combination with pharmaceutical compositions of approved drugs for the treatment of cancers as part of combinatorial multidrug cancer therapy.

Preferably the pharmaceutical composition comprising at least one or two of the compounds of the invention together with at least one approved compound for the treatment of HIV-1 infections are in the same formulation or a pharmaceutically acceptable salt thereof, and suitable excipients and/or diluents to be administered as such.

In the present invention the compounds of the invention or their salts may be administered as pure or as pharmaceutical formulations, i.e. suitable for parenteral, oral, or rectal administrations. Each of said formulations may contain excipients and/or fillers and/or additives and/or binders, coatings and/or suspending agents and/or emulsifying agents, preserving and/or control release agents, suitable for the selected pharmaceutical form.

It is a further object of the invention a method for inhibiting the human DEAD-box RNA helicases DDX3 comprising contacting the compound of the invention or the composition as defined above with human DDX3, thereby inhibiting the activity of DDX3.

It is a further object of the invention a method for treating a viral and/or a hyperproliferative disease in a cell, comprising contacting the cell with the compound or the composition of the invention.

It is a further object of the invention a process for the preparation of the compound as defined above.

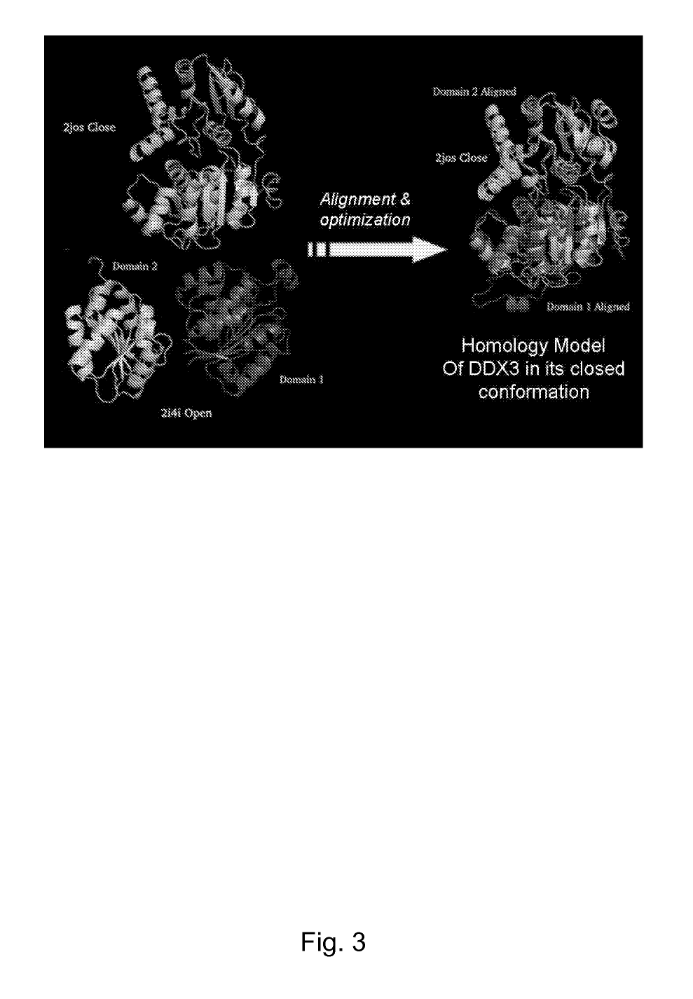

It is a further object of the invention a method for identifying a compound endowed with helicase inhibitory activity against the human DEAD-box RNA helicases DDX3 comprising using at least a portion of the homology model (namely the RNA binding site) of the close conformation of DDX3 as defined in Appendix I and FIG. 3.

Preferably, the method comprises:

(a) Providing a homology model of the close conformation of DDX3 as defined in Appendix I;
(b) providing a candidate compound;
(c) evaluating the level of binding of the candidate compound to the RNA-binding site of the human DEAD-box RNA helicases DDX3, wherein if a sufficient level of binding is found, then the compound is identified.

Preferably the candidate compound is selected from a library of compounds, selected from a from a database, is provided computationally, is designed de novo or is designed from a known DDX3 inhibitor.

Still preferably the sufficient level of binding is indicated by a calculated binding energy defined by a Chemscore value of at least 25.

It is a further object of the invention a compound identified by the method as described above.

Preferably the compound has the formula 4 as defined above.

It is a further object of the invention a composition comprising the compound identified by the method as described above or a pharmaceutically acceptable salt, solvate, or hydrate thereof and pharmaceutically acceptable excipients for use as a treatment of a pathology modulated by DDX3 activity and/or expression.

It is a further object of the invention a computer for producing a three-dimensional representation of the close conformation of DDX3 wherein said computer comprises:

(a) a computer-readable data storage medium comprising a data storage material encoded with computer-readable data, wherein said data comprises the structure co-ordinates of the close conformation of DDX3 of Appendix 1;
(b) a working memory for storing instructions for processing said computer-readable data;
(c) a central-processing unit coupled to said working memory and to said computer-readable data storage medium for processing said computer-machine readable data into said three-dimensional representation; and
(d) a display coupled to said central-processing unit for displaying said three-dimensional representation.

It is a further object of the invention a machine-readable data storage medium comprising a data storage material encoded with machine readable data, wherein the data is defined by at least a portion of the structure co-ordinates of the close conformation of DDX3 of Appendix 1.

It is a further object of the invention the use of the computer or the machine readable data storage medium as defined above to predict the structure and/or function of potential modulators of DDX3.

The foregoing only summarizes certain aspects of the invention and is not intended to be limiting in nature. These and other aspects of the invention will be further elucidated in the following description and examples. The descriptions in the present invention are provided only as examples and should not be understood to be limiting on the claims. Based on the description, a person of ordinary skill in the art could make modifications and changes to the preferred embodiments, which does not depart from the scope of the present invention.

The invention will be now illustrated by means of non limiting examples referring to the following figures

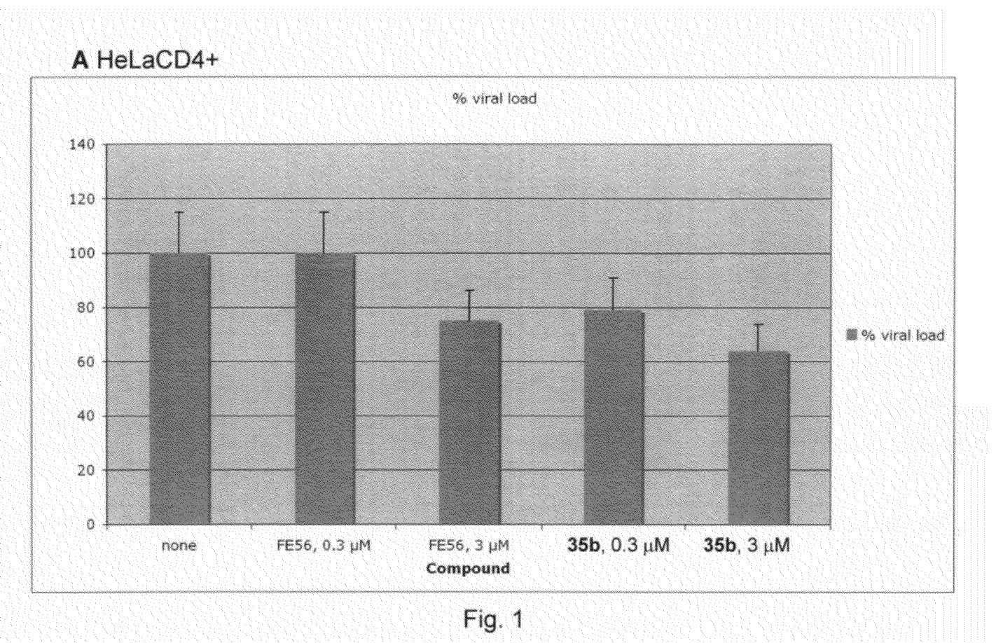

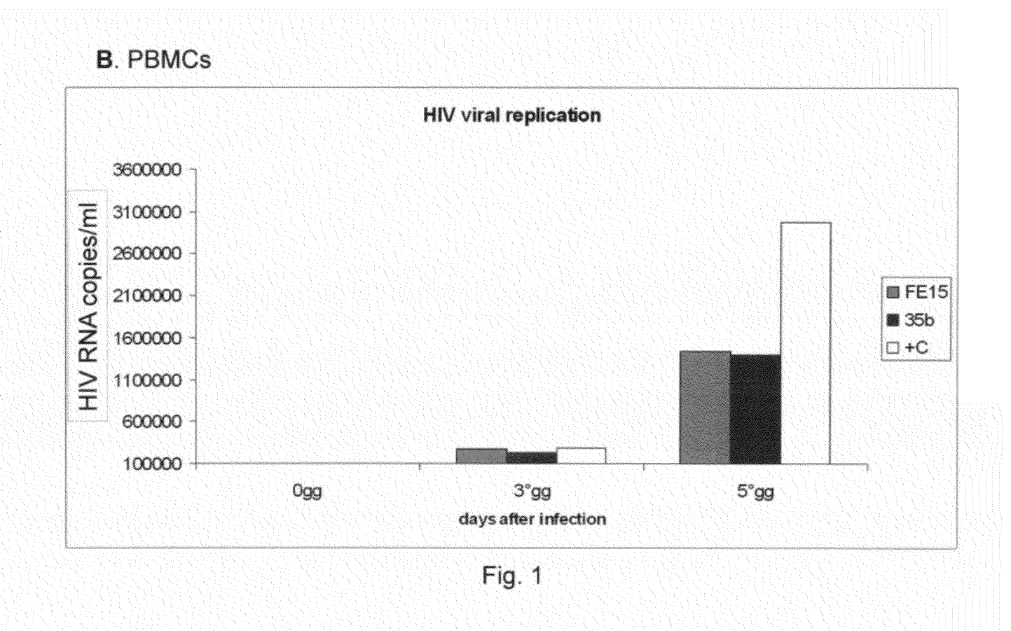

FIG. 1. A) Antiviral activity (expressed as % of viral load with respect to the control) of compounds FE56 and FE66 at 0.3 μM and 3 μM doses against single round HIV-1 replication in HeLaCD4+ cells infected with a wild type HIV-1 strain. Measurements were performed at 72 h post infection. B) Antiviral activity of compounds FE15 and FE66 on HIV-1 replication in phytohemagglutinin-stimulated peripheral blood mononuclear cells (PBMCs) transfected with HeLa CD4⁺-cell culture supernatants containing wild type HIV. Compounds were tested at 50 μM (FE15) and 5 μM (FE66). The HIV-1 RNA was quantified in the cell culture supernatant after 3, and 5 days of incubation. Results are expressed as HIV RNA copies/ml. Control (white bars).

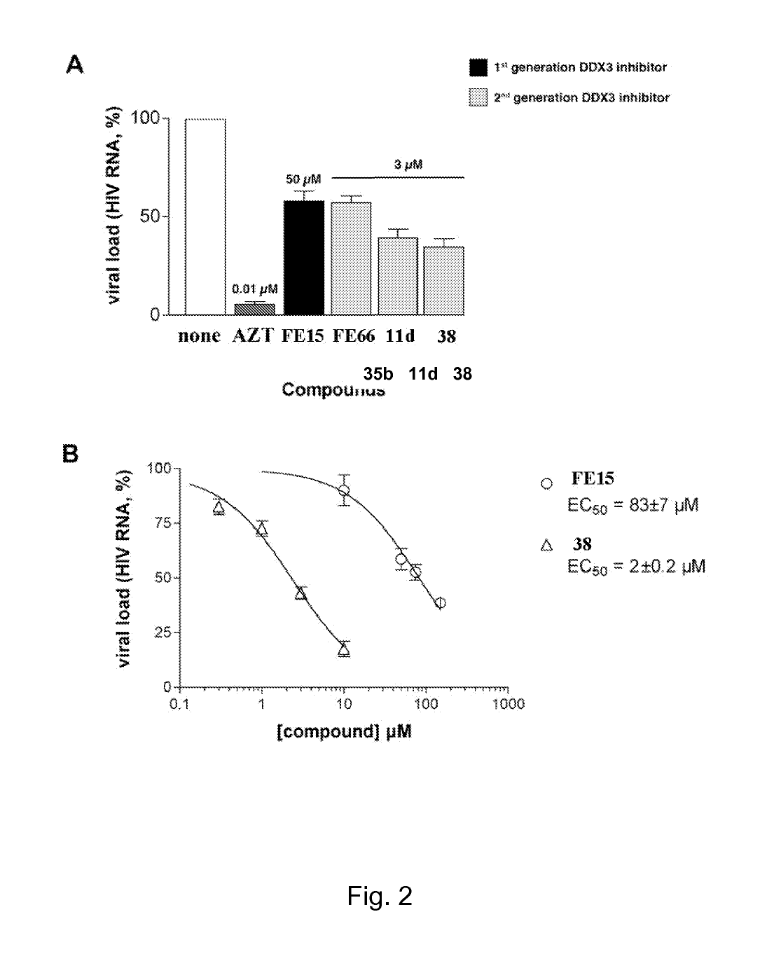

FIG. 2. Antiviral activity of DDX3 inhibitors. A) Comparison of the antiviral activity (expressed as % of viral RNA with respect to the control) on infected PBMCs of a fixed dose of selected inhibitors. Measurements were performed at 72 h post infection. Values are the means of three independent replicates. Error bars are ±SD. White bars: control without drugs; dark grey bar: positive control with AZT (0.01 μM); black bar: first generation inhibitor FE15 (50 μM); light grey bars: second generation DDX3 inhibitors FE66, 11d and 38 (3 μM). B) Dose-response curves for the inhibiton of HIV-1 replication (expressed as % of viral RNA with respect to the control) in infected PBMCs by FE15 (circles) and 38 (triangles). Measurements were performed at 72 h post infection. Values are the means of three independent replicates. Error bars are ±SD.

FIG. 3. Schematic representation of in silico DDX3-closed model preparation.

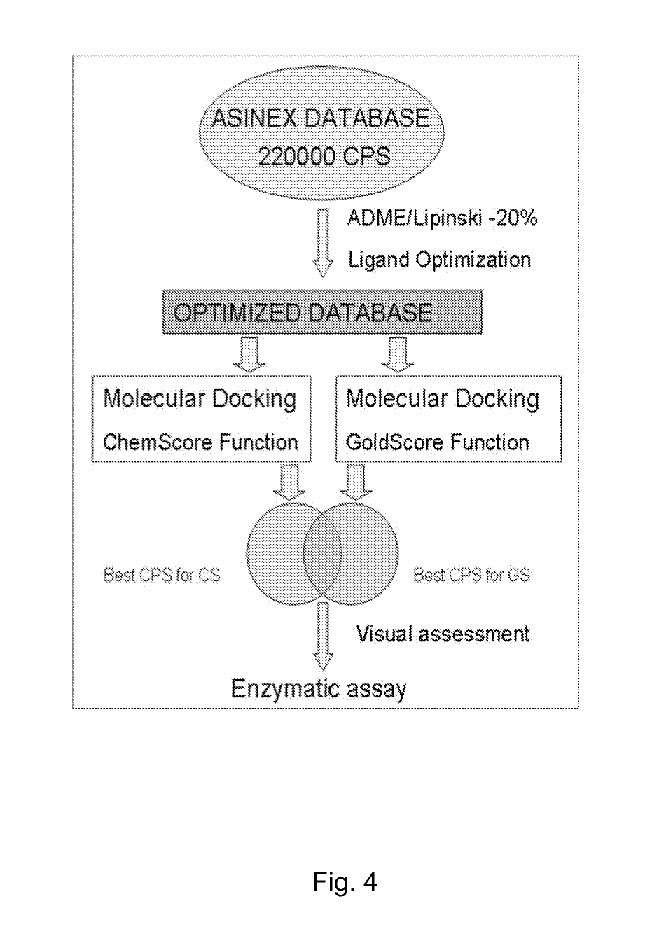

FIG. 4. Schematic representation of in silico screening protocol (virtual docking) for the discovery of DDX3 inhibitors targeting the ATP- and/or the RNA-binding site.

### DETAILED DESCRIPTION OF THE INVENTION

### Material and Methods

### Synthesis

General. All commercially available chemicals were used as purchased. CH₃CN was dried over calcium hydride, tBuOH was dried over Mg/I₂, CH₂Cl₂ was dried over sodium hydride and THF and dioxane were dried over Na/benzophenone prior to use while DMF was bought already anhydrous Anhydrous reactions were run under a positive pressure of dry N₂ or argon. IR spectra were recorded on a Perkin-Elmer BX FTIR system, using KBr pellets. TLC was carried out using Merck TLC plates silica gel 60 F254. Chromatographic purifications were performed on columns packed with Merk 60 silica gel, 23-400 mesh, for flash technique. ¹H-NMR and ¹³C-NMR spectra were recorded at 400 MHz on a Brucker Avance DPX400, at 300 MHz on a Varian VXR-300 and at 200 MHz on a Bruker AC200F spectrometer. Chemical shifts are reported relative to tetramethylsilane at 0.00 ppm. Elemental analyses (C, H, N) were performed in-house using a Perkin-Elmer Elemental Analyzer 240C. Melting points were taken using a Gallenkamp melting point apparatus and are uncorrected. Mass spectra (MS) data were obtained using an Agilent 1100 LC/MSD VL system (G1946C) with a 0.4 mL/min flow rate using a binary solvent system of 95:5 methyl alcohol/water. UV detection was monitored at 254 nm. Mass spectra were acquired in positive and negative mode scanning over the mass range.

Microwave Irradiation Experiments. Microwave irradiation experiments were conducted using a CEM Discover Synthesis Unit (CEM Corp., Matthews, N.C.). The machine consists of a continuous focused microwave power delivery system with operator-selectable power output from 0 to 300 W. The temperature of the contents of the vessels was monitored using a calibrate infrared temperature control mounted under the reaction vessel. All the experiments were performed using a stirring option whereby the contents of the vessel are stirred by means of rotating magnetic plate located below the floor of the microwave cavity and a Teflon-coated magnetic stir bar in the vessel.

### Biology

### Enzymatic Assays

### Human DDX3 ATPase Activity Assay

ATPase activity was tested with a luciferase-based luminescence assay (Easylite-Kinase, Perkin Elmer) on 96 wells microtiter plates. Briefly, recombinant purified human DDX3 (50-100 ng) was incubated in a 15 μL total reaction volume with reaction buffer (25 mM TrisHCl pH 7.5, 5 mM MgCl₂), in the presence of increasing amounts of ATP and/or different combinations of ATP and the inhibitor to be tested. Reference curves in the absence of inhibitor (giving the 100% of enzymatic activity) and in the absence of enzyme (giving the baseline), were also included in each experiment. Reading was performed with a Microbeta Trilux (Perkin Elmer) luminometer, according to the manufacturer's protocol.

### Human DDX3 Helicase Activity Assay

The helicase activity of DDX3 wt and mutant forms was monitored by measuring the conversion of a double stranded (ds) DNA or RNA (labelled at the 5′-end of one strand with a 6-FAM fluorescent group or P³², respectively) into single stranded (ss) nucleic acid. Reactions were performed in 50 mM TrisHCl pH 7.5, 1 mM DTT, 0.2 mg/ml BSA, 5% glycerol and 100 μM ATP, 10 mM MgCl₂ at 37° C. degrees for 10′ and stopped by adding EDTA 50 mM pH 8. Products were separated through non-denaturating 8% PAGE at 5 W for 2 hours in TBE buffer at 4° C. Substrates and products were quantified by laser scanning densitometry (Thyphoon-TRIO, GE Healthcare).

### Kinetic Analysis

ATPase reactions were performed as described, in the presence of increasing amounts of inhibitor and variable ATP concentrations. Variations of the initial velocities of the reaction as a function of ATP concentrations in the absence or presence of 4 were analyzed with the equation:

k_{cat}·E₀/(1+(K_{S}/[ATP])) Eq. (1)

where kₐₜ is the reaction rate, E₀ is the input enzyme concentration, [ATP] is the variable substrate concentration, K_{S} is the apparent affinity for the substrate. K_{S}=Kₘ in the absence of the inhibitor, whereas K_{S}=Kₘ₍ₐₚₚ₎ in the presence of the inhibitor.

Variation of the % inhibition as a function of compound FE15 at different ATP concentrations were fitted to the equation for cooperative binding:

Vₘₐₓ·[I]ⁿ/(Kᵢ₍ₐₚₚ₎+[I]ⁿ) Eq. (2)

where Vₘₐₓ is the apparent maximal rate of the reaction in the absence of the inhibitor, [I] is the variable inhibitor concentration, n is the cooperativity index and Kᵢ₍ₐₚₚ₎ is the apparent inhibition constant. Comparatively, fitting was done with the same Eq. (2) where n was kept fixed at the value of 1 (non-cooperativity hypothesis). F test was used to compare the two fittings and p value was calculated.

The variation of the Kₘ₍ₐₚₚ₎ as a function of the inhibitor concentration was fitted to the equation:

Kₘ₍ₐₚₚ₎=Kₘ/(1+([I]ⁿ/Kᵢ)) Eq. (3)

The variation of the Kᵢ₍ₐₚₚ₎ as a function of the ATP substrate concentration was fitted to the equation:

Kᵢ₍ₐₚₚ₎=Kᵢ·(1+(Kₘ/[ATP]) Eq. (4)

All values used are the means of at least three independent experiments.

### Proteins

Human recombinant DDX3 was cloned, expressed and purified as described (Franca et al. Proteins 2007, 67, 1128-37).

### Cell Viability Assays

Cell viability in the presence of the compounds has been determined by standard MTT assays.

### Antiviral Assays

The susceptibilities of HIV-1 recombinant strains to drugs was performed as follow.

In detail, 0.5 μg of each HIV-1 plasmid construct was transfected into CD4 HeLa cells by using the lipofectin reagent, according to the recommendations of the manufacturer (Invitrogen, Groningen, The Netherlands). After 3 days of incubation at 37° C., the cell supernatants, which contained reconstituted viable recombinant viruses, were collected. Quantification of the newly produced recombinant strains was obtained by determination of the HIV RNA copy number in the cell culture supernatants. Successively, 10 ml of transfected HeLa CD4⁺-cell culture supernatants containing wild-type, and mutated recombinant HIV strain RNA per ml, respectively, was used to infect aliquots of 10×10⁶ phytohemagglutinin-stimulated peripheral blood mononuclear cells (PBMCs) from HIV-seronegative blood donors. After 4 h of incubation, supernatants were removed and infected PBMCs were incubated at 37° C. in 10 ml of RPMI 1640 medium (Eurobio, Les Ulis Cedex B, France) supplemented with 20% fetal calf serum (Life Technologies, Ltd., Paisley, Scotland), 2 mM l-glutamine, 100 U of penicillin per ml, 100 μg of streptomycin per ml, 10% interleukin-2 (ZeptoMetrix Co., Buffalo, N.Y.), 5 μg of hydrocortisone (Sigma Chemical Co.) per ml and with fourfold dilutions of antiretroviral drugs. No-drug controls for each drug dilution were included in each assay. After 3, 5 and 7 days of incubation the HIV-1 RNA in the cell culture supernatant was quantified. Recombinant HIV-1 strains from treatment-naive patients and multidrug resistance-associated changes were assayed in parallel. The degree of inhibition of viral replication was measured by determining the HIV-1 RNA level in the supernatants of cell cultures and was expressed as the fold increase in the 50% inhibitory concentrations (IC₅₀s) for resistant recombinant HIV-1 variants compared with the IC₅₀s for the wild-type recombinant variant. Each test was performed in triplicate.

### Modelling

### Closed Conformation of DDX3

The DDX3X(V168-G582) domain 2 has to rotate approximately 180° relative to domain 1 to obtain the closed conformation of the protein required for RNA binding. This rearrangement would bring positively charged patches on the solvent-exposed surface of domain 2 into closer proximity with positively charged surfaces on domain 1, thus forming the RNA-binding site of the protein.

A comparison of the closed structure of the VASA protein in complex with poly(U) with a modeled closed structure of the DDX3X(V168-G582) protein show that all amino acid residues that are involved in interaction with the RNA in the VASA-structure are present in the DDX3X(V168-G582) structure at corresponding positions. This indicates, as expected, that these residues are involved in RNA binding in both proteins, and that the RNA binding mode in this area should be very similar.

The present homology model has been built as follows: the “closed” structure was built by alignment of the individual domains on the respective domains of the protein eIF4A (eukaryotic translation initiation factor) which is in a closed conformation (pdb entry 2J0S, homology 37%) and final optimization of the resulting structure [RMS 1.2 Å]. 2J0S is cocrystallized with PolyU and this allowed the authors to immediately identify the binding site of nucleic acid for the authors′ protein.

The alignment has been performend using Pymol alignement function. The structure obtained has been used as target for the authors' virtual docking studies. Protein Preparation Wizard procedure was used to obtain a satisfactory starting structure for docking studies. This facility is designed to ensure chemical correctness and to optimize the protein structure for further analysis. The process adds hydrogens, neutralizes appropriate amino acid chains, and relieves steric clashes. In particular, it performs a series of restrained, partial minimizations on the cocrystallized structure, each of which employs a limited number of minimization steps. It is not intended to minimize the system completely. In the present invention, the minimization (OPLS 2001 force field) was stopped when rmsd of the nonhydrogen atoms reached 0.30 Å. The final homology model is reported in Appendix I.

APPENDIX I
Structure coordinates of the Homology Model of DDX3 in the closed
conformation reported in “PDB format” (for further detail see
http://www.wwpdb.org/docs.html)

| | | | | | | | | | | | |
|---|---|---|---|---|---|---|---|---|---|---|---|
| ATOM | 1 | N | MET | A | 167 | 19.061 | 73.704 | 3.313 | 1.00 | 0.00 | N1+ |
| ATOM | 2 | CA | MET | A | 167 | 18.817 | 74.581 | 2.164 | 1.00 | 0.00 | C |
| ATOM | 3 | C | MET | A | 167 | 20.082 | 74.562 | 1.310 | 1.00 | 0.00 | C |
| ATOM | 4 | O | MET | A | 167 | 20.140 | 73.825 | 0.328 | 1.00 | 0.00 | O |
| ATOM | 5 | CB | MET | A | 167 | 18.358 | 75.990 | 2.588 | 1.00 | 0.00 | C |
| ATOM | 6 | CG | MET | A | 167 | 18.124 | 76.936 | 1.403 | 1.00 | 0.00 | C |
| ATOM | 7 | SD | MET | A | 167 | 16.981 | 76.327 | 0.132 | 1.00 | 0.00 | S |
| ATOM | 8 | CE | MET | A | 167 | 17.039 | 77.716 | −1.029 | 1.00 | 0.00 | C |
| ATOM | 9 | 1H | MET | A | 167 | 18.244 | 73.689 | 3.906 | 1.00 | 0.00 | H |
| ATOM | 10 | 2H | MET | A | 167 | 19.258 | 72.769 | 2.988 | 1.00 | 0.00 | H |
| ATOM | 11 | 3H | MET | A | 167 | 19.851 | 74.050 | 3.839 | 1.00 | 0.00 | H |
| ATOM | 12 | HA | MET | A | 167 | 17.899 | 74.275 | 1.661 | 1.00 | 0.00 | H |
| ATOM | 13 | 1HB | MET | A | 167 | 17.421 | 75.917 | 3.139 | 1.00 | 0.00 | H |
| ATOM | 14 | 2HB | MET | A | 167 | 19.118 | 76.443 | 3.224 | 1.00 | 0.00 | H |
| ATOM | 15 | 1HG | MET | A | 167 | 17.709 | 77.877 | 1.765 | 1.00 | 0.00 | H |
| ATOM | 16 | 2HG | MET | A | 167 | 19.071 | 77.128 | 0.898 | 1.00 | 0.00 | H |
| ATOM | 17 | 1HE | MET | A | 167 | 16.388 | 77.508 | −1.878 | 1.00 | 0.00 | H |
| ATOM | 18 | 2HE | MET | A | 167 | 16.703 | 78.624 | −0.528 | 1.00 | 0.00 | H |
| ATOM | 19 | 3HE | MET | A | 167 | 18.061 | 77.854 | −1.381 | 1.00 | 0.00 | H |
| ATOM | 20 | N | VAL | A | 168 | 21.104 | 75.315 | 1.711 | 1.00 | 0.00 | N |
| ATOM | 21 | CA | VAL | A | 168 | 22.421 | 75.342 | 1.104 | 1.00 | 0.00 | C |
| ATOM | 22 | C | VAL | A | 168 | 23.409 | 75.495 | 2.265 | 1.00 | 0.00 | C |
| ATOM | 23 | O | VAL | A | 168 | 23.614 | 76.603 | 2.758 | 1.00 | 0.00 | O |
| ATOM | 24 | CB | VAL | A | 168 | 22.534 | 76.495 | 0.084 | 1.00 | 0.00 | C |
| ATOM | 25 | CG1 | VAL | A | 168 | 23.951 | 76.561 | −0.505 | 1.00 | 0.00 | C |
| ATOM | 26 | CG2 | VAL | A | 168 | 21.550 | 76.321 | −1.082 | 1.00 | 0.00 | C |
| ATOM | 27 | H | VAL | A | 168 | 20.995 | 75.934 | 2.501 | 1.00 | 0.00 | H |
| ATOM | 28 | HA | VAL | A | 168 | 22.578 | 74.427 | 0.532 | 1.00 | 0.00 | H |
| ATOM | 29 | HB | VAL | A | 168 | 22.313 | 77.441 | 0.579 | 1.00 | 0.00 | H |
| ATOM | 30 | 1HG1 | VAL | A | 168 | 24.009 | 77.381 | −1.221 | 1.00 | 0.00 | H |
| ATOM | 31 | 2HG1 | VAL | A | 168 | 24.671 | 76.728 | 0.297 | 1.00 | 0.00 | H |
| ATOM | 32 | 3HG1 | VAL | A | 168 | 24.180 | 75.622 | −1.009 | 1.00 | 0.00 | H |
| ATOM | 33 | 1HG2 | VAL | A | 168 | 21.660 | 77.153 | −1.778 | 1.00 | 0.00 | H |
| ATOM | 34 | 2HG2 | VAL | A | 168 | 21.761 | 75.385 | −1.599 | 1.00 | 0.00 | H |
| ATOM | 35 | 3HG2 | VAL | A | 168 | 20.530 | 76.301 | −0.698 | 1.00 | 0.00 | H |
| ATOM | 36 | N | GLU | A | 169 | 24.029 | 74.393 | 2.689 | 1.00 | 0.00 | N |
| ATOM | 37 | CA | GLU | A | 169 | 24.873 | 74.349 | 3.873 | 1.00 | 0.00 | C |
| ATOM | 38 | C | GLU | A | 169 | 26.127 | 73.524 | 3.555 | 1.00 | 0.00 | C |
| ATOM | 39 | O | GLU | A | 169 | 26.083 | 72.635 | 2.703 | 1.00 | 0.00 | O |
| ATOM | 40 | CB | GLU | A | 169 | 24.099 | 73.754 | 5.066 | 1.00 | 0.00 | C |
| ATOM | 41 | CG | GLU | A | 169 | 22.968 | 74.659 | 5.598 | 1.00 | 0.00 | C |
| ATOM | 42 | CD | GLU | A | 169 | 21.610 | 74.412 | 4.957 | 1.00 | 0.00 | C |
| ATOM | 43 | OE1 | GLU | A | 169 | 21.021 | 75.337 | 4.368 | 1.00 | 0.00 | O |
| ATOM | 44 | OE2 | GLU | A | 169 | 21.080 | 73.287 | 5.025 | 1.00 | 0.00 | O1− |
| ATOM | 45 | H | GLU | A | 169 | 23.924 | 73.527 | 2.179 | 1.00 | 0.00 | H |
| ATOM | 46 | HA | GLU | A | 169 | 25.124 | 75.364 | 4.179 | 1.00 | 0.00 | H |
| ATOM | 47 | 1HB | GLU | A | 169 | 23.641 | 72.810 | 4.769 | 1.00 | 0.00 | H |
| ATOM | 48 | 2HB | GLU | A | 169 | 24.786 | 73.579 | 5.894 | 1.00 | 0.00 | H |
| ATOM | 49 | 1HG | GLU | A | 169 | 22.847 | 74.498 | 6.670 | 1.00 | 0.00 | H |
| ATOM | 50 | 2HG | GLU | A | 169 | 23.220 | 75.703 | 5.414 | 1.00 | 0.00 | H |
| ATOM | 51 | N | ALA | A | 170 | 27.248 | 73.811 | 4.225 | 1.00 | 0.00 | N |
| ATOM | 52 | CA | ALA | A | 170 | 28.504 | 73.082 | 4.115 | 1.00 | 0.00 | C |
| ATOM | 53 | C | ALA | A | 170 | 29.361 | 73.470 | 5.323 | 1.00 | 0.00 | C |
| ATOM | 54 | O | ALA | A | 170 | 30.368 | 74.166 | 5.200 | 1.00 | 0.00 | O |
| ATOM | 55 | CB | ALA | A | 170 | 29.202 | 73.423 | 2.793 | 1.00 | 0.00 | C |
| ATOM | 56 | H | ALA | A | 170 | 27.266 | 74.589 | 4.868 | 1.00 | 0.00 | H |
| ATOM | 57 | HA | ALA | A | 170 | 28.302 | 72.011 | 4.085 | 1.00 | 0.00 | H |
| ATOM | 58 | 1HB | ALA | A | 170 | 30.139 | 72.872 | 2.723 | 1.00 | 0.00 | H |
| ATOM | 59 | 2HB | ALA | A | 170 | 28.556 | 73.148 | 1.959 | 1.00 | 0.00 | H |
| ATOM | 60 | 3HB | ALA | A | 170 | 29.406 | 74.493 | 2.755 | 1.00 | 0.00 | H |
| ATOM | 61 | N | THR | A | 171 | 28.932 | 73.047 | 6.506 | 1.00 | 0.00 | N |
| ATOM | 62 | CA | THR | A | 171 | 29.159 | 73.776 | 7.735 | 1.00 | 0.00 | C |
| ATOM | 63 | C | THR | A | 171 | 29.325 | 72.781 | 8.891 | 1.00 | 0.00 | C |
| ATOM | 64 | O | THR | A | 171 | 28.515 | 71.871 | 9.058 | 1.00 | 0.00 | O |
| ATOM | 65 | CB | THR | A | 171 | 27.929 | 74.681 | 7.966 | 1.00 | 0.00 | C |
| ATOM | 66 | CG2 | THR | A | 171 | 28.219 | 75.769 | 9.004 | 1.00 | 0.00 | C |
| ATOM | 67 | OG1 | THR | A | 171 | 27.509 | 75.308 | 6.760 | 1.00 | 0.00 | O |
| ATOM | 68 | H | THR | A | 171 | 28.420 | 72.179 | 6.577 | 1.00 | 0.00 | H |
| ATOM | 69 | HA | THR | A | 171 | 30.062 | 74.379 | 7.639 | 1.00 | 0.00 | H |
| ATOM | 70 | HB | THR | A | 171 | 27.087 | 74.073 | 8.298 | 1.00 | 0.00 | H |
| ATOM | 71 | 1HG2 | THR | A | 171 | 27.331 | 76.387 | 9.141 | 1.00 | 0.00 | H |
| ATOM | 72 | 2HG2 | THR | A | 171 | 28.487 | 75.304 | 9.952 | 1.00 | 0.00 | H |
| ATOM | 73 | 3HG2 | THR | A | 171 | 29.044 | 76.391 | 8.658 | 1.00 | 0.00 | H |
| ATOM | 74 | HG1 | THR | A | 171 | 26.745 | 75.861 | 6.936 | 1.00 | 0.00 | H |
| ATOM | 75 | N | GLY | A | 172 | 30.358 | 72.950 | 9.712 | 1.00 | 0.00 | N |
| ATOM | 76 | CA | GLY | A | 172 | 30.566 | 72.166 | 10.916 | 1.00 | 0.00 | C |
| ATOM | 77 | C | GLY | A | 172 | 31.878 | 72.623 | 11.544 | 1.00 | 0.00 | C |
| ATOM | 78 | O | GLY | A | 172 | 32.577 | 73.457 | 10.963 | 1.00 | 0.00 | O |
| ATOM | 79 | H | GLY | A | 172 | 31.052 | 73.656 | 9.513 | 1.00 | 0.00 | H |
| ATOM | 80 | 1HA | GLY | A | 172 | 29.738 | 72.330 | 11.605 | 1.00 | 0.00 | H |
| ATOM | 81 | 2HA | GLY | A | 172 | 30.617 | 71.108 | 10.657 | 1.00 | 0.00 | H |
| ATOM | 82 | N | ASN | A | 173 | 32.239 | 72.088 | 12.711 | 1.00 | 0.00 | N |
| ATOM | 83 | CA | ASN | A | 173 | 33.562 | 72.353 | 13.261 | 1.00 | 0.00 | C |
| ATOM | 84 | C | ASN | A | 173 | 34.580 | 71.735 | 12.309 | 1.00 | 0.00 | C |
| ATOM | 85 | O | ASN | A | 173 | 34.499 | 70.537 | 12.047 | 1.00 | 0.00 | O |
| ATOM | 86 | CB | ASN | A | 173 | 33.737 | 71.708 | 14.638 | 1.00 | 0.00 | C |
| ATOM | 87 | CG | ASN | A | 173 | 35.208 | 71.734 | 15.053 | 1.00 | 0.00 | C |
| ATOM | 88 | ND2 | ASN | A | 173 | 35.749 | 70.638 | 15.562 | 1.00 | 0.00 | N |
| ATOM | 89 | OD1 | ASN | A | 173 | 35.893 | 72.734 | 14.856 | 1.00 | 0.00 | O |
| ATOM | 90 | H | ASN | A | 173 | 31.595 | 71.498 | 13.217 | 1.00 | 0.00 | H |
| ATOM | 91 | HA | ASN | A | 173 | 33.726 | 73.429 | 13.314 | 1.00 | 0.00 | H |
| ATOM | 92 | 1HB | ASN | A | 173 | 33.150 | 72.257 | 15.375 | 1.00 | 0.00 | H |
| ATOM | 93 | 2HB | ASN | A | 173 | 33.396 | 70.673 | 14.601 | 1.00 | 0.00 | H |
| ATOM | 94 | 1HD2 | ASN | A | 173 | 36.719 | 70.637 | 15.841 | 1.00 | 0.00 | H |
| ATOM | 95 | 2HD2 | ASN | A | 173 | 35.191 | 69.803 | 15.671 | 1.00 | 0.00 | H |
| ATOM | 96 | N | ASN | A | 174 | 35.544 | 72.518 | 11.827 | 1.00 | 0.00 | N |
| ATOM | 97 | CA | ASN | A | 174 | 36.570 | 72.057 | 10.902 | 1.00 | 0.00 | C |
| ATOM | 98 | C | ASN | A | 174 | 35.911 | 71.331 | 9.728 | 1.00 | 0.00 | C |
| ATOM | 99 | O | ASN | A | 174 | 36.057 | 70.119 | 9.567 | 1.00 | 0.00 | O |
| ATOM | 100 | CB | ASN | A | 174 | 37.616 | 71.191 | 11.625 | 1.00 | 0.00 | C |
| ATOM | 101 | CG | ASN | A | 174 | 38.664 | 72.043 | 12.331 | 1.00 | 0.00 | C |
| ATOM | 102 | ND2 | ASN | A | 174 | 38.362 | 72.575 | 13.510 | 1.00 | 0.00 | N |
| ATOM | 103 | OD1 | ASN | A | 174 | 39.753 | 72.250 | 11.809 | 1.00 | 0.00 | O |
| ATOM | 104 | H | ASN | A | 174 | 35.589 | 73.489 | 12.101 | 1.00 | 0.00 | H |
| ATOM | 105 | HA | ASN | A | 174 | 37.192 | 72.900 | 10.598 | 1.00 | 0.00 | H |
| ATOM | 106 | 1HB | ASN | A | 174 | 37.120 | 70.570 | 12.371 | 1.00 | 0.00 | H |
| ATOM | 107 | 2HB | ASN | A | 174 | 38.124 | 70.554 | 10.901 | 1.00 | 0.00 | H |
| ATOM | 108 | 1HD2 | ASN | A | 174 | 39.041 | 73.143 | 13.996 | 1.00 | 0.00 | H |
| ATOM | 109 | 2HD2 | ASN | A | 174 | 37.454 | 72.412 | 13.920 | 1.00 | 0.00 | H |
| ATOM | 110 | N | CYS | A | 175 | 35.191 | 72.098 | 8.909 | 1.00 | 0.00 | N |
| ATOM | 111 | CA | CYS | A | 175 | 34.717 | 71.648 | 7.610 | 1.00 | 0.00 | C |
| ATOM | 112 | C | CYS | A | 175 | 35.952 | 71.585 | 6.701 | 1.00 | 0.00 | C |
| ATOM | 113 | O | CYS | A | 175 | 36.671 | 72.580 | 6.619 | 1.00 | 0.00 | O |
| ATOM | 114 | CB | CYS | A | 175 | 33.672 | 72.639 | 7.082 | 1.00 | 0.00 | C |
| ATOM | 115 | SG | CYS | A | 175 | 32.855 | 71.965 | 5.610 | 1.00 | 0.00 | S |
| ATOM | 116 | H | CYS | A | 175 | 34.950 | 73.040 | 9.182 | 1.00 | 0.00 | H |
| ATOM | 117 | HA | CYS | A | 175 | 34.244 | 70.672 | 7.714 | 1.00 | 0.00 | H |
| ATOM | 118 | 1HB | CYS | A | 175 | 32.923 | 72.823 | 7.852 | 1.00 | 0.00 | H |
| ATOM | 119 | 2HB | CYS | A | 175 | 34.160 | 73.578 | 6.820 | 1.00 | 0.00 | H |
| ATOM | 120 | HG | CYS | A | 175 | 32.109 | 72.685 | 5.242 | 1.00 | 0.00 | H |
| ATOM | 121 | N | PRO | A | 176 | 36.255 | 70.451 | 6.057 | 1.00 | 0.00 | N |
| ATOM | 122 | CA | PRO | A | 176 | 37.606 | 70.223 | 5.565 | 1.00 | 0.00 | C |
| ATOM | 123 | C | PRO | A | 176 | 37.812 | 70.803 | 4.157 | 1.00 | 0.00 | C |
| ATOM | 124 | O | PRO | A | 176 | 36.854 | 70.934 | 3.392 | 1.00 | 0.00 | O |
| ATOM | 125 | CB | PRO | A | 176 | 37.776 | 68.698 | 5.558 | 1.00 | 0.00 | C |
| ATOM | 126 | CG | PRO | A | 176 | 36.351 | 68.181 | 5.382 | 1.00 | 0.00 | C |
| ATOM | 127 | CD | PRO | A | 176 | 35.542 | 69.192 | 6.190 | 1.00 | 0.00 | C |
| ATOM | 128 | HA | PRO | A | 176 | 38.323 | 70.690 | 6.240 | 1.00 | 0.00 | H |
| ATOM | 129 | 1HB | PRO | A | 176 | 38.428 | 68.408 | 4.734 | 1.00 | 0.00 | H |
| ATOM | 130 | 2HB | PRO | A | 176 | 38.220 | 68.377 | 6.501 | 1.00 | 0.00 | H |
| ATOM | 131 | 1HG | PRO | A | 176 | 36.094 | 68.178 | 4.323 | 1.00 | 0.00 | H |
| ATOM | 132 | 2HG | PRO | A | 176 | 36.281 | 67.167 | 5.775 | 1.00 | 0.00 | H |
| ATOM | 133 | 1HD | PRO | A | 176 | 35.492 | 68.912 | 7.242 | 1.00 | 0.00 | H |
| ATOM | 134 | 2HD | PRO | A | 176 | 34.533 | 69.302 | 5.795 | 1.00 | 0.00 | H |
| ATOM | 135 | N | PRO | A | 177 | 39.061 | 71.128 | 3.779 | 1.00 | 0.00 | N |
| ATOM | 136 | CA | PRO | A | 177 | 39.388 | 71.329 | 2.376 | 1.00 | 0.00 | C |
| ATOM | 137 | C | PRO | A | 177 | 38.974 | 70.105 | 1.546 | 1.00 | 0.00 | C |
| ATOM | 138 | O | PRO | A | 177 | 38.903 | 68.988 | 2.060 | 1.00 | 0.00 | O |
| ATOM | 139 | CB | PRO | A | 177 | 40.903 | 71.537 | 2.345 | 1.00 | 0.00 | C |
| ATOM | 140 | CG | PRO | A | 177 | 41.389 | 70.682 | 3.514 | 1.00 | 0.00 | C |
| ATOM | 141 | CD | PRO | A | 177 | 40.266 | 70.835 | 4.541 | 1.00 | 0.00 | C |
| ATOM | 142 | HA | PRO | A | 177 | 38.863 | 72.208 | 2.003 | 1.00 | 0.00 | H |
| ATOM | 143 | 1HB | PRO | A | 177 | 41.297 | 71.200 | 1.386 | 1.00 | 0.00 | H |
| ATOM | 144 | 2HB | PRO | A | 177 | 41.128 | 72.596 | 2.477 | 1.00 | 0.00 | H |
| ATOM | 145 | 1HG | PRO | A | 177 | 41.517 | 69.652 | 3.183 | 1.00 | 0.00 | H |
| ATOM | 146 | 2HG | PRO | A | 177 | 42.343 | 71.068 | 3.876 | 1.00 | 0.00 | H |
| ATOM | 147 | 1HD | PRO | A | 177 | 40.471 | 71.657 | 5.228 | 1.00 | 0.00 | H |
| ATOM | 148 | 2HD | PRO | A | 177 | 40.124 | 69.915 | 5.108 | 1.00 | 0.00 | H |
| ATOM | 149 | N | HIS | A | 178 | 38.710 | 70.318 | 0.258 | 1.00 | 0.00 | N |
| ATOM | 150 | CA | HIS | A | 178 | 38.026 | 69.341 | −0.571 | 1.00 | 0.00 | C |
| ATOM | 151 | C | HIS | A | 178 | 38.954 | 68.753 | −1.633 | 1.00 | 0.00 | C |
| ATOM | 152 | O | HIS | A | 178 | 39.988 | 69.339 | −1.944 | 1.00 | 0.00 | O |
| ATOM | 153 | CB | HIS | A | 178 | 36.760 | 69.970 | −1.163 | 1.00 | 0.00 | C |
| ATOM | 154 | CG | HIS | A | 178 | 36.913 | 71.164 | −2.070 | 1.00 | 0.00 | C |
| ATOM | 155 | CD2 | HIS | A | 178 | 37.856 | 71.534 | −2.998 | 1.00 | 0.00 | C |
| ATOM | 156 | ND1 | HIS | A | 178 | 35.944 | 72.159 | −2.127 | 1.00 | 0.00 | N |
| ATOM | 157 | CE1 | HIS | A | 178 | 36.286 | 73.030 | −3.088 | 1.00 | 0.00 | C |
| ATOM | 158 | NE2 | HIS | A | 178 | 37.405 | 72.642 | −3.701 | 1.00 | 0.00 | N |
| ATOM | 159 | H | HIS | A | 178 | 38.987 | 71.186 | −0.178 | 1.00 | 0.00 | H |
| ATOM | 160 | HA | HIS | A | 178 | 37.568 | 68.582 | 0.063 | 1.00 | 0.00 | H |
| ATOM | 161 | 1HB | HIS | A | 178 | 36.231 | 69.229 | −1.762 | 1.00 | 0.00 | H |
| ATOM | 162 | 2HB | HIS | A | 178 | 36.112 | 70.312 | −0.356 | 1.00 | 0.00 | H |
| ATOM | 163 | HD2 | HIS | A | 178 | 38.776 | 70.975 | −3.085 | 1.00 | 0.00 | H |
| ATOM | 164 | HD1 | HIS | A | 178 | 35.153 | 72.129 | −1.499 | 1.00 | 0.00 | H |
| ATOM | 165 | HE1 | HIS | A | 178 | 35.675 | 73.902 | −3.270 | 1.00 | 0.00 | H |
| ATOM | 166 | N | ILE | A | 179 | 38.573 | 67.609 | −2.205 | 1.00 | 0.00 | N |
| ATOM | 167 | CA | ILE | A | 179 | 39.376 | 66.922 | −3.213 | 1.00 | 0.00 | C |
| ATOM | 168 | C | ILE | A | 179 | 38.980 | 67.431 | −4.606 | 1.00 | 0.00 | C |
| ATOM | 169 | O | ILE | A | 179 | 38.197 | 68.382 | −4.701 | 1.00 | 0.00 | O |
| ATOM | 170 | CB | ILE | A | 179 | 39.214 | 65.400 | −3.052 | 1.00 | 0.00 | C |
| ATOM | 171 | CG1 | ILE | A | 179 | 37.799 | 64.906 | −3.406 | 1.00 | 0.00 | C |
| ATOM | 172 | CG2 | ILE | A | 179 | 39.636 | 64.967 | −1.640 | 1.00 | 0.00 | C |
| ATOM | 173 | CD1 | ILE | A | 179 | 37.785 | 63.396 | −3.646 | 1.00 | 0.00 | C |
| ATOM | 174 | H | ILE | A | 179 | 37.696 | 67.181 | −1.944 | 1.00 | 0.00 | H |
| ATOM | 175 | HA | ILE | A | 179 | 40.434 | 67.055 | −2.989 | 1.00 | 0.00 | H |
| ATOM | 176 | HB | ILE | A | 179 | 39.987 | 64.889 | −3.626 | 1.00 | 0.00 | H |
| ATOM | 177 | 1HG1 | ILE | A | 179 | 37.118 | 65.134 | −2.585 | 1.00 | 0.00 | H |
| ATOM | 178 | 2HG1 | ILE | A | 179 | 37.455 | 65.406 | −4.311 | 1.00 | 0.00 | H |
| ATOM | 179 | 1HG2 | ILE | A | 179 | 39.516 | 63.888 | −1.539 | 1.00 | 0.00 | H |
| ATOM | 180 | 2HG2 | ILE | A | 179 | 40.680 | 65.234 | −1.474 | 1.00 | 0.00 | H |
| ATOM | 181 | 3HG2 | ILE | A | 179 | 39.011 | 65.472 | −0.903 | 1.00 | 0.00 | H |
| ATOM | 182 | 1HD1 | ILE | A | 179 | 36.773 | 63.076 | −3.894 | 1.00 | 0.00 | H |
| ATOM | 183 | 2HD1 | ILE | A | 179 | 38.455 | 63.153 | −4.472 | 1.00 | 0.00 | H |
| ATOM | 184 | 3HD1 | ILE | A | 179 | 38.118 | 62.881 | −2.745 | 1.00 | 0.00 | H |
| ATOM | 185 | N | GLU | A | 180 | 39.463 | 66.811 | −5.688 | 1.00 | 0.00 | N |
| ATOM | 186 | CA | GLU | A | 180 | 39.273 | 67.302 | −7.047 | 1.00 | 0.00 | C |
| ATOM | 187 | C | GLU | A | 180 | 38.872 | 66.196 | −8.028 | 1.00 | 0.00 | C |
| ATOM | 188 | O | GLU | A | 180 | 38.253 | 66.499 | −9.054 | 1.00 | 0.00 | O |
| ATOM | 189 | CB | GLU | A | 180 | 40.559 | 68.005 | −7.508 | 1.00 | 0.00 | C |
| ATOM | 190 | CG | GLU | A | 180 | 40.514 | 69.513 | −7.221 | 1.00 | 0.00 | C |
| ATOM | 191 | CD | GLU | A | 180 | 39.640 | 70.206 | −8.245 | 1.00 | 0.00 | C |
| ATOM | 192 | OE1 | GLU | A | 180 | 40.110 | 70.374 | −9.388 | 1.00 | 0.00 | O |
| ATOM | 193 | OE2 | GLU | A | 180 | 38.448 | 70.448 | −7.962 | 1.00 | 0.00 | O1− |
| ATOM | 194 | H | GLU | A | 180 | 39.990 | 65.956 | −5.586 | 1.00 | 0.00 | H |
| ATOM | 195 | HA | GLU | A | 180 | 38.531 | 68.100 | −7.045 | 1.00 | 0.00 | H |
| ATOM | 196 | 1HB | GLU | A | 180 | 41.414 | 67.581 | −6.981 | 1.00 | 0.00 | H |
| ATOM | 197 | 2HB | GLU | A | 180 | 40.687 | 67.863 | −8.581 | 1.00 | 0.00 | H |
| ATOM | 198 | 1HG | GLU | A | 180 | 40.104 | 69.683 | −6.225 | 1.00 | 0.00 | H |
| ATOM | 199 | 2HG | GLU | A | 180 | 41.522 | 69.924 | −7.273 | 1.00 | 0.00 | H |
| ATOM | 200 | N | SER | A | 181 | 39.221 | 64.942 | −7.752 | 1.00 | 0.00 | N |
| ATOM | 201 | CA | SER | A | 181 | 39.054 | 63.848 | −8.696 | 1.00 | 0.00 | C |
| ATOM | 202 | C | SER | A | 181 | 38.513 | 62.594 | −7.992 | 1.00 | 0.00 | C |
| ATOM | 203 | O | SER | A | 181 | 37.561 | 62.698 | −7.218 | 1.00 | 0.00 | O |
| ATOM | 204 | CB | SER | A | 181 | 40.368 | 63.656 | −9.466 | 1.00 | 0.00 | C |
| ATOM | 205 | OG | SER | A | 181 | 40.762 | 64.882 | −10.060 | 1.00 | 0.00 | O |
| ATOM | 206 | H | SER | A | 181 | 39.621 | 64.716 | −6.853 | 1.00 | 0.00 | H |
| ATOM | 207 | HA | SER | A | 181 | 38.464 | 64.189 | −9.547 | 1.00 | 0.00 | H |
| ATOM | 208 | 1HB | SER | A | 181 | 41.147 | 63.323 | −8.780 | 1.00 | 0.00 | H |
| ATOM | 209 | 2HB | SER | A | 181 | 40.227 | 62.908 | −10.246 | 1.00 | 0.00 | H |
| ATOM | 210 | HG | SER | A | 181 | 41.584 | 64.756 | −10.540 | 1.00 | 0.00 | H |
| ATOM | 211 | N | PHE | A | 182 | 39.046 | 61.405 | −8.291 | 1.00 | 0.00 | N |
| ATOM | 212 | CA | PHE | A | 182 | 38.544 | 60.132 | −7.786 | 1.00 | 0.00 | C |
| ATOM | 213 | C | PHE | A | 182 | 39.745 | 59.216 | −7.549 | 1.00 | 0.00 | C |
| ATOM | 214 | O | PHE | A | 182 | 39.709 | 58.016 | −7.819 | 1.00 | 0.00 | O |
| ATOM | 215 | CB | PHE | A | 182 | 37.596 | 59.488 | −8.809 | 1.00 | 0.00 | C |
| ATOM | 216 | CG | PHE | A | 182 | 36.305 | 60.221 | −9.120 | 1.00 | 0.00 | C |
| ATOM | 217 | CD1 | PHE | A | 182 | 35.372 | 60.500 | −8.101 | 1.00 | 0.00 | C |
| ATOM | 218 | CD2 | PHE | A | 182 | 35.995 | 60.596 | −10.442 | 1.00 | 0.00 | C |
| ATOM | 219 | CE1 | PHE | A | 182 | 34.135 | 61.095 | −8.402 | 1.00 | 0.00 | C |
| ATOM | 220 | CE2 | PHE | A | 182 | 34.764 | 61.202 | −10.744 | 1.00 | 0.00 | C |
| ATOM | 221 | CZ | PHE | A | 182 | 33.825 | 61.437 | −9.728 | 1.00 | 0.00 | C |
| ATOM | 222 | H | PHE | A | 182 | 39.848 | 61.350 | −8.902 | 1.00 | 0.00 | H |
| ATOM | 223 | HA | PHE | A | 182 | 38.008 | 60.297 | −6.852 | 1.00 | 0.00 | H |
| ATOM | 224 | 1HB | PHE | A | 182 | 38.108 | 59.386 | −9.766 | 1.00 | 0.00 | H |
| ATOM | 225 | 2HB | PHE | A | 182 | 37.291 | 58.504 | −8.453 | 1.00 | 0.00 | H |
| ATOM | 226 | HD1 | PHE | A | 182 | 35.604 | 60.255 | −7.075 | 1.00 | 0.00 | H |
| ATOM | 227 | HD2 | PHE | A | 182 | 36.707 | 60.418 | −11.234 | 1.00 | 0.00 | H |
| ATOM | 228 | HE1 | PHE | A | 182 | 33.423 | 61.289 | −7.614 | 1.00 | 0.00 | H |
| ATOM | 229 | HE2 | PHE | A | 182 | 34.538 | 61.488 | −11.760 | 1.00 | 0.00 | H |
| ATOM | 230 | HZ | PHE | A | 182 | 32.867 | 61.879 | −9.962 | 1.00 | 0.00 | H |
| ATOM | 231 | N | SER | A | 183 | 40.834 | 59.813 | −7.092 | 1.00 | 0.00 | N |
| ATOM | 232 | CA | SER | A | 183 | 42.171 | 59.253 | −7.081 | 1.00 | 0.00 | C |
| ATOM | 233 | C | SER | A | 183 | 42.877 | 59.637 | −5.777 | 1.00 | 0.00 | C |
| ATOM | 234 | O | SER | A | 183 | 44.046 | 59.317 | −5.580 | 1.00 | 0.00 | O |
| ATOM | 235 | CB | SER | A | 183 | 42.917 | 59.848 | −8.290 | 1.00 | 0.00 | C |
| ATOM | 236 | OG | SER | A | 183 | 42.297 | 61.049 | −8.741 | 1.00 | 0.00 | O |
| ATOM | 237 | H | SER | A | 183 | 40.776 | 60.744 | −6.707 | 1.00 | 0.00 | H |
| ATOM | 238 | HA | SER | A | 183 | 42.111 | 58.166 | −7.147 | 1.00 | 0.00 | H |
| ATOM | 239 | 1HB | SER | A | 183 | 43.945 | 60.075 | −8.007 | 1.00 | 0.00 | H |
| ATOM | 240 | 2HB | SER | A | 183 | 42.916 | 59.129 | −9.109 | 1.00 | 0.00 | H |
| ATOM | 241 | HG | SER | A | 183 | 42.782 | 61.397 | −9.493 | 1.00 | 0.00 | H |
| ATOM | 242 | N | ASP | A | 184 | 42.187 | 60.408 | −4.938 | 1.00 | 0.00 | N |
| ATOM | 243 | CA | ASP | A | 184 | 42.820 | 61.525 | −4.255 | 1.00 | 0.00 | C |
| ATOM | 244 | C | ASP | A | 184 | 43.260 | 61.116 | −2.839 | 1.00 | 0.00 | C |
| ATOM | 245 | O | ASP | A | 184 | 43.665 | 61.964 | −2.051 | 1.00 | 0.00 | O |
| ATOM | 246 | CB | ASP | A | 184 | 41.847 | 62.729 | −4.249 | 1.00 | 0.00 | C |
| ATOM | 247 | CG | ASP | A | 184 | 41.400 | 63.229 | −5.628 | 1.00 | 0.00 | C |
| ATOM | 248 | OD1 | ASP | A | 184 | 41.348 | 62.406 | −6.568 | 1.00 | 0.00 | O |
| ATOM | 249 | OD2 | ASP | A | 184 | 41.035 | 64.420 | −5.744 | 1.00 | 0.00 | O1− |
| ATOM | 250 | H | ASP | A | 184 | 41.209 | 60.220 | −4.768 | 1.00 | 0.00 | H |
| ATOM | 251 | HA | ASP | A | 184 | 43.652 | 61.894 | −4.855 | 1.00 | 0.00 | H |
| ATOM | 252 | 1HB | ASP | A | 184 | 40.937 | 62.459 | −3.713 | 1.00 | 0.00 | H |
| ATOM | 253 | 2HB | ASP | A | 184 | 42.320 | 63.578 | −3.755 | 1.00 | 0.00 | H |
| ATOM | 254 | N | VAL | A | 185 | 43.171 | 59.818 | −2.523 | 1.00 | 0.00 | N |
| ATOM | 255 | CA | VAL | A | 185 | 43.515 | 59.163 | −1.259 | 1.00 | 0.00 | C |
| ATOM | 256 | C | VAL | A | 185 | 43.609 | 57.651 | −1.556 | 1.00 | 0.00 | C |
| ATOM | 257 | O | VAL | A | 185 | 43.545 | 57.275 | −2.730 | 1.00 | 0.00 | O |
| ATOM | 258 | CB | VAL | A | 185 | 42.475 | 59.472 | −0.153 | 1.00 | 0.00 | C |
| ATOM | 259 | CG1 | VAL | A | 185 | 42.580 | 60.897 | 0.405 | 1.00 | 0.00 | C |
| ATOM | 260 | CG2 | VAL | A | 185 | 41.030 | 59.213 | −0.600 | 1.00 | 0.00 | C |
| ATOM | 261 | H | VAL | A | 185 | 42.829 | 59.155 | −3.204 | 1.00 | 0.00 | H |
| ATOM | 262 | HA | VAL | A | 185 | 44.445 | 59.580 | −0.874 | 1.00 | 0.00 | H |
| ATOM | 263 | HB | VAL | A | 185 | 42.556 | 58.730 | 0.640 | 1.00 | 0.00 | H |
| ATOM | 264 | 1HG1 | VAL | A | 185 | 41.822 | 61.043 | 1.175 | 1.00 | 0.00 | H |
| ATOM | 265 | 2HG1 | VAL | A | 185 | 43.569 | 61.046 | 0.838 | 1.00 | 0.00 | H |
| ATOM | 266 | 3HG1 | VAL | A | 185 | 42.424 | 61.616 | −0.399 | 1.00 | 0.00 | H |
| ATOM | 267 | 1HG2 | VAL | A | 185 | 40.348 | 59.448 | 0.217 | 1.00 | 0.00 | H |
| ATOM | 268 | 2HG2 | VAL | A | 185 | 40.796 | 59.841 | −1.460 | 1.00 | 0.00 | H |
| ATOM | 269 | 3HG2 | VAL | A | 185 | 40.917 | 58.164 | −0.876 | 1.00 | 0.00 | H |
| ATOM | 270 | N | GLU | A | 186 | 43.739 | 56.768 | −0.553 | 1.00 | 0.00 | N |
| ATOM | 271 | CA | GLU | A | 186 | 43.956 | 55.342 | −0.780 | 1.00 | 0.00 | C |
| ATOM | 272 | C | GLU | A | 186 | 42.799 | 54.737 | −1.586 | 1.00 | 0.00 | C |
| ATOM | 273 | O | GLU | A | 186 | 41.740 | 54.418 | −1.041 | 1.00 | 0.00 | O |
| ATOM | 274 | CB | GLU | A | 186 | 44.152 | 54.543 | 0.522 | 1.00 | 0.00 | C |
| ATOM | 275 | CG | GLU | A | 186 | 45.092 | 55.183 | 1.552 | 1.00 | 0.00 | C |
| ATOM | 276 | CD | GLU | A | 186 | 44.273 | 55.982 | 2.538 | 1.00 | 0.00 | C |
| ATOM | 277 | OE1 | GLU | A | 186 | 43.834 | 57.077 | 2.145 | 1.00 | 0.00 | O |
| ATOM | 278 | OE2 | GLU | A | 186 | 43.895 | 55.433 | 3.596 | 1.00 | 0.00 | O1− |
| ATOM | 279 | H | GLU | A | 186 | 43.688 | 57.082 | 0.406 | 1.00 | 0.00 | H |
| ATOM | 280 | HA | GLU | A | 186 | 44.914 | 55.195 | −1.280 | 1.00 | 0.00 | H |
| ATOM | 281 | 1HB | GLU | A | 186 | 43.190 | 54.417 | 1.020 | 1.00 | 0.00 | H |
| ATOM | 282 | 2HB | GLU | A | 186 | 44.571 | 53.564 | 0.289 | 1.00 | 0.00 | H |
| ATOM | 283 | 1HG | GLU | A | 186 | 45.640 | 54.402 | 2.079 | 1.00 | 0.00 | H |
| ATOM | 284 | 2HG | GLU | A | 186 | 45.796 | 55.841 | 1.043 | 1.00 | 0.00 | H |
| ATOM | 285 | N | MET | A | 187 | 43.019 | 54.553 | −2.883 | 1.00 | 0.00 | N |
| ATOM | 286 | CA | MET | A | 187 | 42.015 | 54.197 | −3.867 | 1.00 | 0.00 | C |
| ATOM | 287 | C | MET | A | 187 | 42.792 | 53.744 | −5.098 | 1.00 | 0.00 | C |
| ATOM | 288 | O | MET | A | 187 | 42.866 | 52.553 | −5.396 | 1.00 | 0.00 | O |
| ATOM | 289 | CB | MET | A | 187 | 41.114 | 55.404 | −4.201 | 1.00 | 0.00 | C |
| ATOM | 290 | CG | MET | A | 187 | 40.115 | 55.763 | −3.093 | 1.00 | 0.00 | C |
| ATOM | 291 | SD | MET | A | 187 | 38.765 | 56.880 | −3.567 | 1.00 | 0.00 | S |
| ATOM | 292 | CE | MET | A | 187 | 39.719 | 58.291 | −4.177 | 1.00 | 0.00 | C |
| ATOM | 293 | H | MET | A | 187 | 43.951 | 54.660 | −3.257 | 1.00 | 0.00 | H |
| ATOM | 294 | HA | MET | A | 187 | 41.388 | 53.397 | −3.474 | 1.00 | 0.00 | H |
| ATOM | 295 | 1HB | MET | A | 187 | 41.733 | 56.285 | −4.373 | 1.00 | 0.00 | H |
| ATOM | 296 | 2HB | MET | A | 187 | 40.534 | 55.188 | −5.099 | 1.00 | 0.00 | H |
| ATOM | 297 | 1HG | MET | A | 187 | 39.639 | 54.855 | −2.724 | 1.00 | 0.00 | H |
| ATOM | 298 | 2HG | MET | A | 187 | 40.641 | 56.255 | −2.275 | 1.00 | 0.00 | H |
| ATOM | 299 | 1HE | MET | A | 187 | 39.037 | 59.074 | −4.509 | 1.00 | 0.00 | H |
| ATOM | 300 | 2HE | MET | A | 187 | 40.351 | 58.676 | −3.376 | 1.00 | 0.00 | H |
| ATOM | 301 | 3HE | MET | A | 187 | 40.343 | 57.974 | −5.012 | 1.00 | 0.00 | H |
| ATOM | 302 | N | GLY | A | 188 | 43.423 | 54.707 | −5.775 | 1.00 | 0.00 | N |
| ATOM | 303 | CA | GLY | A | 188 | 44.259 | 54.473 | −6.938 | 1.00 | 0.00 | C |
| ATOM | 304 | C | GLY | A | 188 | 43.602 | 53.496 | −7.914 | 1.00 | 0.00 | C |
| ATOM | 305 | O | GLY | A | 188 | 42.441 | 53.675 | −8.277 | 1.00 | 0.00 | O |
| ATOM | 306 | H | GLY | A | 188 | 43.333 | 55.670 | −5.484 | 1.00 | 0.00 | H |
| ATOM | 307 | 1HA | GLY | A | 188 | 44.431 | 55.416 | −7.458 | 1.00 | 0.00 | H |
| ATOM | 308 | 2HA | GLY | A | 188 | 45.214 | 54.054 | −6.621 | 1.00 | 0.00 | H |
| ATOM | 309 | N | GLU | A | 189 | 44.351 | 52.471 | −8.327 | 1.00 | 0.00 | N |
| ATOM | 310 | CA | GLU | A | 189 | 43.938 | 51.434 | −9.264 | 1.00 | 0.00 | C |
| ATOM | 311 | C | GLU | A | 189 | 42.536 | 50.908 | −8.928 | 1.00 | 0.00 | C |
| ATOM | 312 | O | GLU | A | 189 | 41.707 | 50.685 | −9.812 | 1.00 | 0.00 | O |
| ATOM | 313 | CB | GLU | A | 189 | 44.992 | 50.311 | −9.214 | 1.00 | 0.00 | C |
| ATOM | 314 | CG | GLU | A | 189 | 45.132 | 49.536 | −10.529 | 1.00 | 0.00 | C |
| ATOM | 315 | CD | GLU | A | 189 | 43.867 | 48.796 | −10.913 | 1.00 | 0.00 | C |
| ATOM | 316 | OE1 | GLU | A | 189 | 43.566 | 48.744 | −12.121 | 1.00 | 0.00 | O |
| ATOM | 317 | OE2 | GLU | A | 189 | 43.172 | 48.286 | −10.012 | 1.00 | 0.00 | O1− |
| ATOM | 318 | H | GLU | A | 189 | 45.295 | 52.365 | −7.985 | 1.00 | 0.00 | H |
| ATOM | 319 | HA | GLU | A | 189 | 43.966 | 51.830 | −10.280 | 1.00 | 0.00 | H |
| ATOM | 320 | 1HB | GLU | A | 189 | 45.968 | 50.738 | −8.985 | 1.00 | 0.00 | H |
| ATOM | 321 | 2HB | GLU | A | 189 | 44.720 | 49.591 | −8.442 | 1.00 | 0.00 | H |
| ATOM | 322 | 1HG | GLU | A | 189 | 45.369 | 50.229 | −11.336 | 1.00 | 0.00 | H |
| ATOM | 323 | 2HG | GLU | A | 189 | 45.931 | 48.801 | −10.435 | 1.00 | 0.00 | H |
| ATOM | 324 | N | ILE | A | 190 | 42.264 | 50.706 | −7.639 | 1.00 | 0.00 | N |
| ATOM | 325 | CA | ILE | A | 190 | 41.107 | 49.941 | −7.210 | 1.00 | 0.00 | C |
| ATOM | 326 | C | ILE | A | 190 | 39.827 | 50.736 | −7.504 | 1.00 | 0.00 | C |
| ATOM | 327 | O | ILE | A | 190 | 38.729 | 50.178 | −7.510 | 1.00 | 0.00 | O |
| ATOM | 328 | CB | ILE | A | 190 | 41.248 | 49.523 | −5.728 | 1.00 | 0.00 | C |
| ATOM | 329 | CG1 | ILE | A | 190 | 42.669 | 49.001 | −5.420 | 1.00 | 0.00 | C |
| ATOM | 330 | CG2 | ILE | A | 190 | 40.225 | 48.424 | −5.404 | 1.00 | 0.00 | C |
| ATOM | 331 | CD1 | ILE | A | 190 | 42.857 | 48.537 | −3.971 | 1.00 | 0.00 | C |
| ATOM | 332 | H | ILE | A | 190 | 42.871 | 51.090 | −6.929 | 1.00 | 0.00 | H |
| ATOM | 333 | HA | ILE | A | 190 | 41.134 | 48.952 | −7.669 | 1.00 | 0.00 | H |
| ATOM | 334 | HB | ILE | A | 190 | 41.033 | 50.379 | −5.088 | 1.00 | 0.00 | H |
| ATOM | 335 | 1HG1 | ILE | A | 190 | 42.891 | 48.150 | −6.064 | 1.00 | 0.00 | H |
| ATOM | 336 | 2HG1 | ILE | A | 190 | 43.395 | 49.793 | −5.602 | 1.00 | 0.00 | H |
| ATOM | 337 | 1HG2 | ILE | A | 190 | 40.325 | 48.131 | −4.359 | 1.00 | 0.00 | H |
| ATOM | 338 | 2HG2 | ILE | A | 190 | 39.218 | 48.801 | −5.580 | 1.00 | 0.00 | H |
| ATOM | 339 | 3HG2 | ILE | A | 190 | 40.406 | 47.559 | −6.042 | 1.00 | 0.00 | H |
| ATOM | 340 | 1HD1 | ILE | A | 190 | 43.878 | 48.184 | −3.832 | 1.00 | 0.00 | H |
| ATOM | 341 | 2HD1 | ILE | A | 190 | 42.665 | 49.369 | −3.295 | 1.00 | 0.00 | H |
| ATOM | 342 | 3HD1 | ILE | A | 190 | 42.160 | 47.726 | −3.756 | 1.00 | 0.00 | H |
| ATOM | 343 | N | ILE | A | 191 | 39.964 | 52.035 | −7.771 | 1.00 | 0.00 | N |
| ATOM | 344 | CA | ILE | A | 191 | 38.884 | 52.892 | −8.218 | 1.00 | 0.00 | C |
| ATOM | 345 | C | ILE | A | 191 | 39.142 | 53.270 | −9.674 | 1.00 | 0.00 | C |
| ATOM | 346 | O | ILE | A | 191 | 38.284 | 53.043 | −10.521 | 1.00 | 0.00 | O |
| ATOM | 347 | CB | ILE | A | 191 | 38.747 | 54.112 | −7.292 | 1.00 | 0.00 | C |
| ATOM | 348 | CG1 | ILE | A | 191 | 38.412 | 53.699 | −5.844 | 1.00 | 0.00 | C |
| ATOM | 349 | CG2 | ILE | A | 191 | 37.710 | 55.114 | −7.821 | 1.00 | 0.00 | C |
| ATOM | 350 | CD1 | ILE | A | 191 | 37.130 | 52.876 | −5.673 | 1.00 | 0.00 | C |
| ATOM | 351 | H | ILE | A | 191 | 40.865 | 52.480 | −7.666 | 1.00 | 0.00 | H |
| ATOM | 352 | HA | ILE | A | 191 | 37.931 | 52.387 | −8.060 | 1.00 | 0.00 | H |
| ATOM | 353 | HB | ILE | A | 191 | 39.656 | 54.711 | −7.342 | 1.00 | 0.00 | H |
| ATOM | 354 | 1HG1 | ILE | A | 191 | 39.222 | 53.091 | −5.442 | 1.00 | 0.00 | H |
| ATOM | 355 | 2HG1 | ILE | A | 191 | 38.288 | 54.591 | −5.230 | 1.00 | 0.00 | H |
| ATOM | 356 | 1HG2 | ILE | A | 191 | 37.642 | 55.962 | −7.140 | 1.00 | 0.00 | H |
| ATOM | 357 | 2HG2 | ILE | A | 191 | 38.013 | 55.464 | −8.808 | 1.00 | 0.00 | H |
| ATOM | 358 | 3HG2 | ILE | A | 191 | 36.737 | 54.627 | −7.892 | 1.00 | 0.00 | H |
| ATOM | 359 | 1HD1 | ILE | A | 191 | 36.988 | 52.639 | −4.619 | 1.00 | 0.00 | H |
| ATOM | 360 | 2HD1 | ILE | A | 191 | 36.278 | 53.452 | −6.034 | 1.00 | 0.00 | H |
| ATOM | 361 | 3HD1 | ILE | A | 191 | 37.212 | 51.952 | −6.246 | 1.00 | 0.00 | H |
| ATOM | 362 | N | MET | A | 192 | 40.305 | 53.847 | −9.972 | 1.00 | 0.00 | N |
| ATOM | 363 | CA | MET | A | 192 | 40.636 | 54.366 | −11.292 | 1.00 | 0.00 | C |
| ATOM | 364 | C | MET | A | 192 | 40.480 | 53.279 | −12.354 | 1.00 | 0.00 | C |
| ATOM | 365 | O | MET | A | 192 | 39.865 | 53.508 | −13.392 | 1.00 | 0.00 | O |
| ATOM | 366 | CB | MET | A | 192 | 42.055 | 54.949 | −11.301 | 1.00 | 0.00 | C |
| ATOM | 367 | CG | MET | A | 192 | 42.188 | 56.192 | −10.409 | 1.00 | 0.00 | C |
| ATOM | 368 | SD | MET | A | 192 | 41.172 | 57.628 | −10.858 | 1.00 | 0.00 | S |
| ATOM | 369 | CE | MET | A | 192 | 41.852 | 58.040 | −12.487 | 1.00 | 0.00 | C |
| ATOM | 370 | H | MET | A | 192 | 41.018 | 53.945 | −9.263 | 1.00 | 0.00 | H |
| ATOM | 371 | HA | MET | A | 192 | 40.013 | 55.234 | −11.508 | 1.00 | 0.00 | H |
| ATOM | 372 | 1HB | MET | A | 192 | 42.758 | 54.200 | −10.938 | 1.00 | 0.00 | H |
| ATOM | 373 | 2HB | MET | A | 192 | 42.323 | 55.237 | −12.318 | 1.00 | 0.00 | H |
| ATOM | 374 | 1HG | MET | A | 192 | 41.907 | 55.939 | −9.387 | 1.00 | 0.00 | H |
| ATOM | 375 | 2HG | MET | A | 192 | 43.220 | 56.543 | −10.425 | 1.00 | 0.00 | H |
| ATOM | 376 | 1HE | MET | A | 192 | 41.327 | 58.905 | −12.892 | 1.00 | 0.00 | H |
| ATOM | 377 | 2HE | MET | A | 192 | 42.912 | 58.272 | −12.390 | 1.00 | 0.00 | H |
| ATOM | 378 | 3HE | MET | A | 192 | 41.725 | 57.192 | −13.159 | 1.00 | 0.00 | H |
| ATOM | 379 | N | GLY | A | 193 | 40.974 | 52.076 | −12.075 | 1.00 | 0.00 | N |
| ATOM | 380 | CA | GLY | A | 193 | 40.907 | 50.950 | −12.992 | 1.00 | 0.00 | C |
| ATOM | 381 | C | GLY | A | 193 | 39.532 | 50.282 | −12.944 | 1.00 | 0.00 | C |
| ATOM | 382 | O | GLY | A | 193 | 39.355 | 49.169 | −13.433 | 1.00 | 0.00 | O |
| ATOM | 383 | H | GLY | A | 193 | 41.424 | 51.909 | −11.187 | 1.00 | 0.00 | H |
| ATOM | 384 | 1HA | GLY | A | 193 | 41.089 | 51.298 | −14.009 | 1.00 | 0.00 | H |
| ATOM | 385 | 2HA | GLY | A | 193 | 41.662 | 50.213 | −12.720 | 1.00 | 0.00 | H |
| ATOM | 386 | N | ASN | A | 194 | 38.550 | 50.952 | −12.340 | 1.00 | 0.00 | N |
| ATOM | 387 | CA | ASN | A | 194 | 37.149 | 50.575 | −12.332 | 1.00 | 0.00 | C |
| ATOM | 388 | C | ASN | A | 194 | 36.312 | 51.769 | −12.808 | 1.00 | 0.00 | C |
| ATOM | 389 | O | ASN | A | 194 | 35.100 | 51.644 | −12.995 | 1.00 | 0.00 | O |
| ATOM | 390 | CB | ASN | A | 194 | 36.739 | 50.126 | −10.921 | 1.00 | 0.00 | C |
| ATOM | 391 | CG | ASN | A | 194 | 37.197 | 48.705 | −10.599 | 1.00 | 0.00 | C |
| ATOM | 392 | ND2 | ASN | A | 194 | 37.734 | 48.452 | −9.406 | 1.00 | 0.00 | N |
| ATOM | 393 | OD1 | ASN | A | 194 | 37.103 | 47.806 | −11.423 | 1.00 | 0.00 | O |
| ATOM | 394 | H | ASN | A | 194 | 38.755 | 51.800 | −11.832 | 1.00 | 0.00 | H |
| ATOM | 395 | HA | ASN | A | 194 | 37.003 | 49.701 | −12.967 | 1.00 | 0.00 | H |
| ATOM | 396 | 1HB | ASN | A | 194 | 37.183 | 50.795 | −10.183 | 1.00 | 0.00 | H |
| ATOM | 397 | 2HB | ASN | A | 194 | 35.653 | 50.155 | −10.831 | 1.00 | 0.00 | H |
| ATOM | 398 | 1HD2 | ASN | A | 194 | 38.042 | 47.518 | −9.178 | 1.00 | 0.00 | H |
| ATOM | 399 | 2HD2 | ASN | A | 194 | 37.834 | 49.196 | −8.729 | 1.00 | 0.00 | H |
| ATOM | 400 | N | ILE | A | 195 | 36.926 | 52.933 | −13.059 | 1.00 | 0.00 | N |
| ATOM | 401 | CA | ILE | A | 195 | 36.215 | 54.053 | −13.658 | 1.00 | 0.00 | C |
| ATOM | 402 | C | ILE | A | 195 | 35.805 | 53.659 | −15.076 | 1.00 | 0.00 | C |
| ATOM | 403 | O | ILE | A | 195 | 34.788 | 54.138 | −15.569 | 1.00 | 0.00 | O |
| ATOM | 404 | CB | ILE | A | 195 | 37.019 | 55.371 | −13.598 | 1.00 | 0.00 | C |
| ATOM | 405 | CG1 | ILE | A | 195 | 37.060 | 55.861 | −12.140 | 1.00 | 0.00 | C |
| ATOM | 406 | CG2 | ILE | A | 195 | 36.400 | 56.467 | −14.481 | 1.00 | 0.00 | C |
| ATOM | 407 | CD1 | ILE | A | 195 | 37.848 | 57.159 | −11.954 | 1.00 | 0.00 | C |
| ATOM | 408 | H | ILE | A | 195 | 37.904 | 53.047 | −12.832 | 1.00 | 0.00 | H |
| ATOM | 409 | HA | ILE | A | 195 | 35.395 | 54.354 | −13.007 | 1.00 | 0.00 | H |
| ATOM | 410 | HB | ILE | A | 195 | 38.029 | 55.198 | −13.969 | 1.00 | 0.00 | H |
| ATOM | 411 | 1HG1 | ILE | A | 195 | 36.044 | 56.044 | −11.789 | 1.00 | 0.00 | H |
| ATOM | 412 | 2HG1 | ILE | A | 195 | 37.529 | 55.103 | −11.514 | 1.00 | 0.00 | H |
| ATOM | 413 | 1HG2 | ILE | A | 195 | 36.999 | 57.375 | −14.407 | 1.00 | 0.00 | H |
| ATOM | 414 | 2HG2 | ILE | A | 195 | 36.378 | 56.131 | −15.517 | 1.00 | 0.00 | H |
| ATOM | 415 | 3HG2 | ILE | A | 195 | 35.384 | 56.674 | −14.145 | 1.00 | 0.00 | H |
| ATOM | 416 | 1HD1 | ILE | A | 195 | 37.834 | 57.445 | −10.902 | 1.00 | 0.00 | H |
| ATOM | 417 | 2HD1 | ILE | A | 195 | 38.878 | 57.009 | −12.275 | 1.00 | 0.00 | H |
| ATOM | 418 | 3HD1 | ILE | A | 195 | 37.393 | 57.950 | −12.551 | 1.00 | 0.00 | H |
| ATOM | 419 | N | GLU | A | 196 | 36.527 | 52.740 | −15.714 | 1.00 | 0.00 | N |
| ATOM | 420 | CA | GLU | A | 196 | 36.171 | 52.244 | −17.033 | 1.00 | 0.00 | C |
| ATOM | 421 | C | GLU | A | 196 | 34.872 | 51.419 | −17.009 | 1.00 | 0.00 | C |
| ATOM | 422 | O | GLU | A | 196 | 34.374 | 51.010 | −18.061 | 1.00 | 0.00 | O |
| ATOM | 423 | CB | GLU | A | 196 | 37.356 | 51.464 | −17.619 | 1.00 | 0.00 | C |
| ATOM | 424 | CG | GLU | A | 196 | 38.440 | 52.431 | −18.121 | 1.00 | 0.00 | C |
| ATOM | 425 | CD | GLU | A | 196 | 37.953 | 53.147 | −19.368 | 1.00 | 0.00 | C |
| ATOM | 426 | OE1 | GLU | A | 196 | 37.883 | 52.471 | −20.414 | 1.00 | 0.00 | O |
| ATOM | 427 | OE2 | GLU | A | 196 | 37.444 | 54.278 | −19.250 | 1.00 | 0.00 | O1− |
| ATOM | 428 | H | GLU | A | 196 | 37.358 | 52.361 | −15.282 | 1.00 | 0.00 | H |
| ATOM | 429 | HA | GLU | A | 196 | 36.124 | 53.077 | −17.735 | 1.00 | 0.00 | H |
| ATOM | 430 | 1HB | GLU | A | 196 | 37.783 | 50.821 | −16.850 | 1.00 | 0.00 | H |
| ATOM | 431 | 2HB | GLU | A | 196 | 37.012 | 50.854 | −18.454 | 1.00 | 0.00 | H |
| ATOM | 432 | 1HG | GLU | A | 196 | 38.659 | 53.166 | −17.346 | 1.00 | 0.00 | H |
| ATOM | 433 | 2HG | GLU | A | 196 | 39.345 | 51.872 | −18.356 | 1.00 | 0.00 | H |
| ATOM | 434 | N | LEU | A | 197 | 34.289 | 51.199 | −15.829 | 1.00 | 0.00 | N |
| ATOM | 435 | CA | LEU | A | 197 | 32.974 | 50.596 | −15.671 | 1.00 | 0.00 | C |
| ATOM | 436 | C | LEU | A | 197 | 31.936 | 51.695 | −15.409 | 1.00 | 0.00 | C |
| ATOM | 437 | O | LEU | A | 197 | 30.807 | 51.395 | −15.029 | 1.00 | 0.00 | O |
| ATOM | 438 | CB | LEU | A | 197 | 32.996 | 49.589 | −14.509 | 1.00 | 0.00 | C |
| ATOM | 439 | CG | LEU | A | 197 | 34.124 | 48.547 | −14.601 | 1.00 | 0.00 | C |
| ATOM | 440 | CD1 | LEU | A | 197 | 34.105 | 47.668 | −13.347 | 1.00 | 0.00 | C |
| ATOM | 441 | CD2 | LEU | A | 197 | 33.985 | 47.663 | −15.846 | 1.00 | 0.00 | C |
| ATOM | 442 | H | LEU | A | 197 | 34.765 | 51.456 | −14.975 | 1.00 | 0.00 | H |
| ATOM | 443 | HA | LEU | A | 197 | 32.721 | 50.034 | −16.570 | 1.00 | 0.00 | H |
| ATOM | 444 | 1HB | LEU | A | 197 | 33.130 | 50.123 | −13.567 | 1.00 | 0.00 | H |
| ATOM | 445 | 2HB | LEU | A | 197 | 32.055 | 49.041 | −14.484 | 1.00 | 0.00 | H |
| ATOM | 446 | HG | LEU | A | 197 | 35.083 | 49.056 | −14.701 | 1.00 | 0.00 | H |
| ATOM | 447 | 1HD1 | LEU | A | 197 | 34.903 | 46.928 | −13.409 | 1.00 | 0.00 | H |
| ATOM | 448 | 2HD1 | LEU | A | 197 | 34.255 | 48.290 | −12.465 | 1.00 | 0.00 | H |
| ATOM | 449 | 3HD1 | LEU | A | 197 | 33.144 | 47.160 | −13.273 | 1.00 | 0.00 | H |
| ATOM | 450 | 1HD2 | LEU | A | 197 | 34.802 | 46.941 | −15.873 | 1.00 | 0.00 | H |
| ATOM | 451 | 2HD2 | LEU | A | 197 | 33.033 | 47.133 | −15.812 | 1.00 | 0.00 | H |
| ATOM | 452 | 3HD2 | LEU | A | 197 | 34.022 | 48.285 | −16.741 | 1.00 | 0.00 | H |
| ATOM | 453 | N | THR | A | 198 | 32.324 | 52.967 | −15.562 | 1.00 | 0.00 | N |
| ATOM | 454 | CA | THR | A | 198 | 31.583 | 54.099 | −15.025 | 1.00 | 0.00 | C |
| ATOM | 455 | C | THR | A | 198 | 31.760 | 55.357 | −15.892 | 1.00 | 0.00 | C |
| ATOM | 456 | O | THR | A | 198 | 30.857 | 55.729 | −16.634 | 1.00 | 0.00 | O |
| ATOM | 457 | CB | THR | A | 198 | 32.019 | 54.335 | −13.564 | 1.00 | 0.00 | C |
| ATOM | 458 | CG2 | THR | A | 198 | 31.038 | 55.274 | −12.866 | 1.00 | 0.00 | C |
| ATOM | 459 | OG1 | THR | A | 198 | 32.080 | 53.126 | −12.834 | 1.00 | 0.00 | O |
| ATOM | 460 | H | THR | A | 198 | 33.169 | 53.179 | −16.073 | 1.00 | 0.00 | H |
| ATOM | 461 | HA | THR | A | 198 | 30.533 | 53.828 | −14.914 | 1.00 | 0.00 | H |
| ATOM | 462 | HB | THR | A | 198 | 33.018 | 54.771 | −13.548 | 1.00 | 0.00 | H |
| ATOM | 463 | 1HG2 | THR | A | 198 | 31.357 | 55.432 | −11.836 | 1.00 | 0.00 | H |
| ATOM | 464 | 2HG2 | THR | A | 198 | 31.014 | 56.229 | −13.390 | 1.00 | 0.00 | H |
| ATOM | 465 | 3HG2 | THR | A | 198 | 30.042 | 54.830 | −12.874 | 1.00 | 0.00 | H |
| ATOM | 466 | HG1 | THR | A | 198 | 32.354 | 53.309 | −11.932 | 1.00 | 0.00 | H |
| ATOM | 467 | N | ARG | A | 199 | 32.920 | 56.011 | −15.806 | 1.00 | 0.00 | N |
| ATOM | 468 | CA | ARG | A | 199 | 33.281 | 57.232 | −16.514 | 1.00 | 0.00 | C |
| ATOM | 469 | C | ARG | A | 199 | 32.465 | 58.437 | −16.038 | 1.00 | 0.00 | C |
| ATOM | 470 | O | ARG | A | 199 | 32.035 | 59.259 | −16.842 | 1.00 | 0.00 | O |
| ATOM | 471 | CB | ARG | A | 199 | 33.273 | 57.037 | −18.039 | 1.00 | 0.00 | C |
| ATOM | 472 | CG | ARG | A | 199 | 34.168 | 55.852 | −18.427 | 1.00 | 0.00 | C |
| ATOM | 473 | CD | ARG | A | 199 | 34.241 | 55.660 | −19.946 | 1.00 | 0.00 | C |
| ATOM | 474 | NE | ARG | A | 199 | 34.983 | 54.432 | −20.274 | 1.00 | 0.00 | N |
| ATOM | 475 | CZ | ARG | A | 199 | 34.517 | 53.185 | −20.168 | 1.00 | 0.00 | C |
| ATOM | 476 | NH1 | ARG | A | 199 | 33.274 | 52.984 | −19.729 | 1.00 | 0.00 | N1+ |
| ATOM | 477 | NH2 | ARG | A | 199 | 35.282 | 52.142 | −20.460 | 1.00 | 0.00 | N |
| ATOM | 478 | H | ARG | A | 199 | 33.652 | 55.664 | −15.202 | 1.00 | 0.00 | H |
| ATOM | 479 | HA | ARG | A | 199 | 34.363 | 57.360 | −16.490 | 1.00 | 0.00 | H |
| ATOM | 480 | 1HB | ARG | A | 199 | 32.255 | 56.840 | −18.376 | 1.00 | 0.00 | H |
| ATOM | 481 | 2HB | ARG | A | 199 | 33.648 | 57.939 | −18.523 | 1.00 | 0.00 | H |
| ATOM | 482 | 1HG | ARG | A | 199 | 35.179 | 56.023 | −18.057 | 1.00 | 0.00 | H |
| ATOM | 483 | 2HG | ARG | A | 199 | 33.771 | 54.937 | −17.987 | 1.00 | 0.00 | H |
| ATOM | 484 | 1HD | ARG | A | 199 | 33.233 | 55.583 | −20.352 | 1.00 | 0.00 | H |
| ATOM | 485 | 2HD | ARG | A | 199 | 34.750 | 56.512 | −20.397 | 1.00 | 0.00 | H |
| ATOM | 486 | HE | ARG | A | 199 | 35.923 | 54.593 | −20.606 | 1.00 | 0.00 | H |
| ATOM | 487 | 1HH1 | ARG | A | 199 | 32.914 | 52.044 | −19.645 | 1.00 | 0.00 | H |
| ATOM | 488 | 2HH1 | ARG | A | 199 | 32.692 | 53.771 | −19.480 | 1.00 | 0.00 | H |
| ATOM | 489 | 1HH2 | ARG | A | 199 | 34.913 | 51.206 | −20.373 | 1.00 | 0.00 | H |
| ATOM | 490 | 2HH2 | ARG | A | 199 | 36.233 | 52.284 | −20.768 | 1.00 | 0.00 | H |
| ATOM | 491 | N | TYR | A | 200 | 32.360 | 58.599 | −14.718 | 1.00 | 0.00 | N |
| ATOM | 492 | CA | TYR | A | 200 | 32.104 | 59.912 | −14.145 | 1.00 | 0.00 | C |
| ATOM | 493 | C | TYR | A | 200 | 33.428 | 60.673 | −14.244 | 1.00 | 0.00 | C |
| ATOM | 494 | O | TYR | A | 200 | 34.476 | 60.092 | −13.964 | 1.00 | 0.00 | O |
| ATOM | 495 | CB | TYR | A | 200 | 31.682 | 59.800 | −12.671 | 1.00 | 0.00 | C |
| ATOM | 496 | CG | TYR | A | 200 | 30.539 | 58.853 | −12.348 | 1.00 | 0.00 | C |
| ATOM | 497 | CD1 | TYR | A | 200 | 29.485 | 58.626 | −13.256 | 1.00 | 0.00 | C |
| ATOM | 498 | CD2 | TYR | A | 200 | 30.519 | 58.177 | −11.112 | 1.00 | 0.00 | C |
| ATOM | 499 | CE1 | TYR | A | 200 | 28.457 | 57.717 | −12.948 | 1.00 | 0.00 | C |
| ATOM | 500 | CE2 | TYR | A | 200 | 29.489 | 57.274 | −10.801 | 1.00 | 0.00 | C |
| ATOM | 501 | CZ | TYR | A | 200 | 28.460 | 57.023 | −11.725 | 1.00 | 0.00 | C |
| ATOM | 502 | OH | TYR | A | 200 | 27.480 | 56.116 | −11.443 | 1.00 | 0.00 | O |
| ATOM | 503 | H | TYR | A | 200 | 32.458 | 57.804 | −14.102 | 1.00 | 0.00 | H |
| ATOM | 504 | HA | TYR | A | 200 | 31.332 | 60.418 | −14.724 | 1.00 | 0.00 | H |
| ATOM | 505 | 1HB | TYR | A | 200 | 32.526 | 59.451 | −12.076 | 1.00 | 0.00 | H |
| ATOM | 506 | 2HB | TYR | A | 200 | 31.362 | 60.777 | −12.308 | 1.00 | 0.00 | H |
| ATOM | 507 | HD1 | TYR | A | 200 | 29.461 | 59.152 | −14.199 | 1.00 | 0.00 | H |
| ATOM | 508 | HD2 | TYR | A | 200 | 31.303 | 58.351 | −10.390 | 1.00 | 0.00 | H |
| ATOM | 509 | HE1 | TYR | A | 200 | 27.657 | 57.548 | −13.653 | 1.00 | 0.00 | H |
| ATOM | 510 | HE2 | TYR | A | 200 | 29.485 | 56.768 | −9.847 | 1.00 | 0.00 | H |
| ATOM | 511 | HH | TYR | A | 200 | 27.634 | 55.738 | −10.574 | 1.00 | 0.00 | H |
| ATOM | 512 | N | THR | A | 201 | 33.408 | 61.940 | −14.648 | 1.00 | 0.00 | N |
| ATOM | 513 | CA | THR | A | 201 | 34.606 | 62.659 | −15.052 | 1.00 | 0.00 | C |
| ATOM | 514 | C | THR | A | 201 | 35.003 | 63.733 | −14.031 | 1.00 | 0.00 | C |
| ATOM | 515 | O | THR | A | 201 | 35.990 | 64.437 | −14.234 | 1.00 | 0.00 | O |
| ATOM | 516 | CB | THR | A | 201 | 34.302 | 63.278 | −16.426 | 1.00 | 0.00 | C |
| ATOM | 517 | CG2 | THR | A | 201 | 34.368 | 62.209 | −17.523 | 1.00 | 0.00 | C |
| ATOM | 518 | OG1 | THR | A | 201 | 32.986 | 63.813 | −16.420 | 1.00 | 0.00 | O |
| ATOM | 519 | H | THR | A | 201 | 32.534 | 62.445 | −14.683 | 1.00 | 0.00 | H |
| ATOM | 520 | HA | THR | A | 201 | 35.426 | 61.954 | −15.183 | 1.00 | 0.00 | H |
| ATOM | 521 | HB | THR | A | 201 | 35.050 | 64.036 | −16.657 | 1.00 | 0.00 | H |
| ATOM | 522 | 1HG2 | THR | A | 201 | 34.150 | 62.665 | −18.489 | 1.00 | 0.00 | H |
| ATOM | 523 | 2HG2 | THR | A | 201 | 35.366 | 61.772 | −17.545 | 1.00 | 0.00 | H |
| ATOM | 524 | 3HG2 | THR | A | 201 | 33.634 | 61.430 | −17.316 | 1.00 | 0.00 | H |
| ATOM | 525 | HG1 | THR | A | 201 | 32.793 | 64.200 | −17.277 | 1.00 | 0.00 | H |
| ATOM | 526 | N | ARG | A | 202 | 34.229 | 63.910 | −12.958 | 1.00 | 0.00 | N |
| ATOM | 527 | CA | ARG | A | 202 | 34.471 | 64.940 | −11.963 | 1.00 | 0.00 | C |
| ATOM | 528 | C | ARG | A | 202 | 33.552 | 64.651 | −10.771 | 1.00 | 0.00 | C |
| ATOM | 529 | O | ARG | A | 202 | 32.389 | 64.321 | −11.000 | 1.00 | 0.00 | O |
| ATOM | 530 | CB | ARG | A | 202 | 34.119 | 66.311 | −12.571 | 1.00 | 0.00 | C |
| ATOM | 531 | CG | ARG | A | 202 | 34.620 | 67.515 | −11.763 | 1.00 | 0.00 | C |
| ATOM | 532 | CD | ARG | A | 202 | 36.138 | 67.723 | −11.893 | 1.00 | 0.00 | C |
| ATOM | 533 | NE | ARG | A | 202 | 36.512 | 69.084 | −11.473 | 1.00 | 0.00 | N |
| ATOM | 534 | CZ | ARG | A | 202 | 37.710 | 69.473 | −11.007 | 1.00 | 0.00 | C |
| ATOM | 535 | NH1 | ARG | A | 202 | 38.660 | 68.594 | −10.708 | 1.00 | 0.00 | N1+ |
| ATOM | 536 | NH2 | ARG | A | 202 | 37.955 | 70.770 | −10.854 | 1.00 | 0.00 | N |
| ATOM | 537 | H | ARG | A | 202 | 33.430 | 63.311 | −12.808 | 1.00 | 0.00 | H |
| ATOM | 538 | HA | ARG | A | 202 | 35.511 | 64.896 | −11.638 | 1.00 | 0.00 | H |
| ATOM | 539 | 1HB | ARG | A | 202 | 34.560 | 66.392 | −13.564 | 1.00 | 0.00 | H |
| ATOM | 540 | 2HB | ARG | A | 202 | 33.036 | 66.409 | −12.645 | 1.00 | 0.00 | H |
| ATOM | 541 | 1HG | ARG | A | 202 | 34.127 | 68.421 | −12.116 | 1.00 | 0.00 | H |
| ATOM | 542 | 2HG | ARG | A | 202 | 34.392 | 67.366 | −10.707 | 1.00 | 0.00 | H |
| ATOM | 543 | 1HD | ARG | A | 202 | 36.659 | 67.003 | −11.261 | 1.00 | 0.00 | H |
| ATOM | 544 | 2HD | ARG | A | 202 | 36.438 | 67.579 | −12.931 | 1.00 | 0.00 | H |
| ATOM | 545 | HE | ARG | A | 202 | 35.763 | 69.755 | −11.563 | 1.00 | 0.00 | H |
| ATOM | 546 | 1HH1 | ARG | A | 202 | 39.552 | 68.914 | −10.359 | 1.00 | 0.00 | H |
| ATOM | 547 | 2HH1 | ARG | A | 202 | 38.490 | 67.606 | −10.830 | 1.00 | 0.00 | H |
| ATOM | 548 | 1HH2 | ARG | A | 202 | 38.851 | 71.076 | −10.504 | 1.00 | 0.00 | H |
| ATOM | 549 | 2HH2 | ARG | A | 202 | 37.245 | 71.449 | −11.087 | 1.00 | 0.00 | H |
| ATOM | 550 | N | PRO | A | 203 | 34.011 | 64.763 | −9.518 | 1.00 | 0.00 | N |
| ATOM | 551 | CA | PRO | A | 203 | 33.125 | 64.663 | −8.373 | 1.00 | 0.00 | C |
| ATOM | 552 | C | PRO | A | 203 | 32.154 | 65.851 | −8.359 | 1.00 | 0.00 | C |
| ATOM | 553 | O | PRO | A | 203 | 32.542 | 66.990 | −8.626 | 1.00 | 0.00 | O |
| ATOM | 554 | CB | PRO | A | 203 | 34.037 | 64.649 | −7.141 | 1.00 | 0.00 | C |
| ATOM | 555 | CG | PRO | A | 203 | 35.308 | 65.348 | −7.619 | 1.00 | 0.00 | C |
| ATOM | 556 | CD | PRO | A | 203 | 35.387 | 64.934 | −9.086 | 1.00 | 0.00 | C |
| ATOM | 557 | HA | PRO | A | 203 | 32.572 | 63.725 | −8.421 | 1.00 | 0.00 | H |
| ATOM | 558 | 1HB | PRO | A | 203 | 33.554 | 65.186 | −6.325 | 1.00 | 0.00 | H |
| ATOM | 559 | 2HB | PRO | A | 203 | 34.223 | 63.619 | −6.838 | 1.00 | 0.00 | H |
| ATOM | 560 | 1HG | PRO | A | 203 | 35.201 | 66.425 | −7.491 | 1.00 | 0.00 | H |
| ATOM | 561 | 2HG | PRO | A | 203 | 36.158 | 64.996 | −7.036 | 1.00 | 0.00 | H |
| ATOM | 562 | 1HD | PRO | A | 203 | 35.925 | 63.993 | −9.205 | 1.00 | 0.00 | H |
| ATOM | 563 | 2HD | PRO | A | 203 | 35.865 | 65.704 | −9.692 | 1.00 | 0.00 | H |
| ATOM | 564 | N | THR | A | 204 | 30.893 | 65.583 | −8.036 | 1.00 | 0.00 | N |
| ATOM | 565 | CA | THR | A | 204 | 29.847 | 66.584 | −7.973 | 1.00 | 0.00 | C |
| ATOM | 566 | C | THR | A | 204 | 30.030 | 67.465 | −6.719 | 1.00 | 0.00 | C |
| ATOM | 567 | O | THR | A | 204 | 30.884 | 67.169 | −5.879 | 1.00 | 0.00 | O |
| ATOM | 568 | CB | THR | A | 204 | 28.505 | 65.834 | −7.951 | 1.00 | 0.00 | C |
| ATOM | 569 | CG2 | THR | A | 204 | 28.314 | 64.958 | −9.196 | 1.00 | 0.00 | C |
| ATOM | 570 | OG1 | THR | A | 204 | 28.440 | 65.017 | −6.796 | 1.00 | 0.00 | O |
| ATOM | 571 | H | THR | A | 204 | 30.618 | 64.636 | −7.816 | 1.00 | 0.00 | H |
| ATOM | 572 | HA | THR | A | 204 | 29.873 | 67.195 | −8.876 | 1.00 | 0.00 | H |
| ATOM | 573 | HB | THR | A | 204 | 27.687 | 66.552 | −7.892 | 1.00 | 0.00 | H |
| ATOM | 574 | 1HG2 | THR | A | 204 | 27.353 | 64.447 | −9.137 | 1.00 | 0.00 | H |
| ATOM | 575 | 2HG2 | THR | A | 204 | 28.338 | 65.584 | −10.088 | 1.00 | 0.00 | H |
| ATOM | 576 | 3HG2 | THR | A | 204 | 29.114 | 64.220 | −9.248 | 1.00 | 0.00 | H |
| ATOM | 577 | HG1 | THR | A | 204 | 27.602 | 64.549 | −6.782 | 1.00 | 0.00 | H |
| ATOM | 578 | N | PRO | A | 205 | 29.222 | 68.522 | −6.515 | 1.00 | 0.00 | N |
| ATOM | 579 | CA | PRO | A | 205 | 29.477 | 69.462 | −5.430 | 1.00 | 0.00 | C |
| ATOM | 580 | C | PRO | A | 205 | 29.371 | 68.813 | −4.047 | 1.00 | 0.00 | C |
| ATOM | 581 | O | PRO | A | 205 | 30.072 | 69.205 | −3.116 | 1.00 | 0.00 | O |
| ATOM | 582 | CB | PRO | A | 205 | 28.463 | 70.592 | −5.610 | 1.00 | 0.00 | C |
| ATOM | 583 | CG | PRO | A | 205 | 28.256 | 70.609 | −7.124 | 1.00 | 0.00 | C |
| ATOM | 584 | CD | PRO | A | 205 | 28.307 | 69.123 | −7.479 | 1.00 | 0.00 | C |
| ATOM | 585 | HA | PRO | A | 205 | 30.466 | 69.904 | −5.557 | 1.00 | 0.00 | H |
| ATOM | 586 | 1HB | PRO | A | 205 | 27.552 | 70.354 | −5.061 | 1.00 | 0.00 | H |
| ATOM | 587 | 2HB | PRO | A | 205 | 28.883 | 71.522 | −5.230 | 1.00 | 0.00 | H |
| ATOM | 588 | 1HG | PRO | A | 205 | 27.294 | 71.066 | −7.355 | 1.00 | 0.00 | H |
| ATOM | 589 | 2HG | PRO | A | 205 | 29.054 | 71.184 | −7.594 | 1.00 | 0.00 | H |
| ATOM | 590 | 1HD | PRO | A | 205 | 28.688 | 68.968 | −8.488 | 1.00 | 0.00 | H |
| ATOM | 591 | 2HD | PRO | A | 205 | 27.325 | 68.658 | −7.387 | 1.00 | 0.00 | H |
| ATOM | 592 | N | VAL | A | 206 | 28.512 | 67.802 | −3.918 | 1.00 | 0.00 | N |
| ATOM | 593 | CA | VAL | A | 206 | 28.405 | 67.041 | −2.686 | 1.00 | 0.00 | C |
| ATOM | 594 | C | VAL | A | 206 | 29.623 | 66.124 | −2.570 | 1.00 | 0.00 | C |
| ATOM | 595 | O | VAL | A | 206 | 30.285 | 66.101 | −1.536 | 1.00 | 0.00 | O |
| ATOM | 596 | CB | VAL | A | 206 | 27.049 | 66.310 | −2.578 | 1.00 | 0.00 | C |
| ATOM | 597 | CG1 | VAL | A | 206 | 26.705 | 65.407 | −3.770 | 1.00 | 0.00 | C |
| ATOM | 598 | CG2 | VAL | A | 206 | 26.984 | 65.470 | −1.296 | 1.00 | 0.00 | C |
| ATOM | 599 | H | VAL | A | 206 | 27.913 | 67.547 | −4.691 | 1.00 | 0.00 | H |
| ATOM | 600 | HA | VAL | A | 206 | 28.250 | 67.723 | −1.849 | 1.00 | 0.00 | H |
| ATOM | 601 | HB | VAL | A | 206 | 26.252 | 67.040 | −2.439 | 1.00 | 0.00 | H |
| ATOM | 602 | 1HG1 | VAL | A | 206 | 25.736 | 64.937 | −3.602 | 1.00 | 0.00 | H |
| ATOM | 603 | 2HG1 | VAL | A | 206 | 26.667 | 66.004 | −4.680 | 1.00 | 0.00 | H |
| ATOM | 604 | 3HG1 | VAL | A | 206 | 27.469 | 64.636 | −3.874 | 1.00 | 0.00 | H |
| ATOM | 605 | 1HG2 | VAL | A | 206 | 26.020 | 64.963 | −1.242 | 1.00 | 0.00 | H |
| ATOM | 606 | 2HG2 | VAL | A | 206 | 27.783 | 64.728 | −1.305 | 1.00 | 0.00 | H |
| ATOM | 607 | 3HG2 | VAL | A | 206 | 27.102 | 66.119 | −0.429 | 1.00 | 0.00 | H |
| ATOM | 608 | N | GLN | A | 207 | 29.925 | 65.369 | −3.627 | 1.00 | 0.00 | N |
| ATOM | 609 | CA | GLN | A | 207 | 31.005 | 64.396 | −3.629 | 1.00 | 0.00 | C |
| ATOM | 610 | C | GLN | A | 207 | 32.336 | 65.058 | −3.274 | 1.00 | 0.00 | C |
| ATOM | 611 | O | GLN | A | 207 | 33.113 | 64.493 | −2.509 | 1.00 | 0.00 | O |
| ATOM | 612 | CB | GLN | A | 207 | 31.071 | 63.717 | −4.998 | 1.00 | 0.00 | C |
| ATOM | 613 | CG | GLN | A | 207 | 29.864 | 62.794 | −5.211 | 1.00 | 0.00 | C |
| ATOM | 614 | CD | GLN | A | 207 | 29.741 | 62.299 | −6.644 | 1.00 | 0.00 | C |
| ATOM | 615 | NE2 | GLN | A | 207 | 28.753 | 61.456 | −6.924 | 1.00 | 0.00 | N |
| ATOM | 616 | OE1 | GLN | A | 207 | 30.535 | 62.663 | −7.504 | 1.00 | 0.00 | O |
| ATOM | 617 | H | GLN | A | 207 | 29.391 | 65.458 | −4.480 | 1.00 | 0.00 | H |
| ATOM | 618 | HA | GLN | A | 207 | 30.769 | 63.589 | −2.936 | 1.00 | 0.00 | H |
| ATOM | 619 | 1HB | GLN | A | 207 | 31.075 | 64.475 | −5.781 | 1.00 | 0.00 | H |
| ATOM | 620 | 2HB | GLN | A | 207 | 31.982 | 63.122 | −5.065 | 1.00 | 0.00 | H |
| ATOM | 621 | 1HG | GLN | A | 207 | 29.955 | 61.921 | −4.565 | 1.00 | 0.00 | H |
| ATOM | 622 | 2HG | GLN | A | 207 | 28.948 | 63.332 | −4.967 | 1.00 | 0.00 | H |
| ATOM | 623 | 1HE2 | GLN | A | 207 | 28.640 | 61.105 | −7.864 | 1.00 | 0.00 | H |
| ATOM | 624 | 2HE2 | GLN | A | 207 | 28.115 | 61.166 | −6.197 | 1.00 | 0.00 | H |
| ATOM | 625 | N | LYS | A | 208 | 32.591 | 66.247 | −3.824 | 1.00 | 0.00 | N |
| ATOM | 626 | CA | LYS | A | 208 | 33.777 | 67.041 | −3.530 | 1.00 | 0.00 | C |
| ATOM | 627 | C | LYS | A | 208 | 34.098 | 67.062 | −2.029 | 1.00 | 0.00 | C |
| ATOM | 628 | O | LYS | A | 208 | 35.270 | 67.063 | −1.656 | 1.00 | 0.00 | O |
| ATOM | 629 | CB | LYS | A | 208 | 33.562 | 68.481 | −4.027 | 1.00 | 0.00 | C |
| ATOM | 630 | CG | LYS | A | 208 | 33.764 | 68.660 | −5.544 | 1.00 | 0.00 | C |
| ATOM | 631 | CD | LYS | A | 208 | 35.226 | 68.945 | −5.934 | 1.00 | 0.00 | C |
| ATOM | 632 | CE | LYS | A | 208 | 35.670 | 70.374 | −5.560 | 1.00 | 0.00 | C |
| ATOM | 633 | NZ | LYS | A | 208 | 37.118 | 70.595 | −5.742 | 1.00 | 0.00 | N1+ |
| ATOM | 634 | H | LYS | A | 208 | 31.942 | 66.647 | −4.486 | 1.00 | 0.00 | H |
| ATOM | 635 | HA | LYS | A | 208 | 34.629 | 66.637 | −4.076 | 1.00 | 0.00 | H |
| ATOM | 636 | 1HB | LYS | A | 208 | 32.544 | 68.796 | −3.800 | 1.00 | 0.00 | H |
| ATOM | 637 | 2HB | LYS | A | 208 | 34.268 | 69.147 | −3.529 | 1.00 | 0.00 | H |
| ATOM | 638 | 1HG | LYS | A | 208 | 33.457 | 67.751 | −6.062 | 1.00 | 0.00 | H |
| ATOM | 639 | 2HG | LYS | A | 208 | 33.161 | 69.498 | −5.895 | 1.00 | 0.00 | H |
| ATOM | 640 | 1HD | LYS | A | 208 | 35.884 | 68.245 | −5.419 | 1.00 | 0.00 | H |
| ATOM | 641 | 2HD | LYS | A | 208 | 35.345 | 68.829 | −7.012 | 1.00 | 0.00 | H |
| ATOM | 642 | 1HE | LYS | A | 208 | 35.144 | 71.094 | −6.187 | 1.00 | 0.00 | H |
| ATOM | 643 | 2HE | LYS | A | 208 | 35.435 | 70.566 | −4.513 | 1.00 | 0.00 | H |
| ATOM | 644 | 1HZ | LYS | A | 208 | 37.350 | 71.543 | −5.483 | 1.00 | 0.00 | H |
| ATOM | 645 | 2HZ | LYS | A | 208 | 37.633 | 69.951 | −5.158 | 1.00 | 0.00 | H |
| ATOM | 646 | 3HZ | LYS | A | 208 | 37.364 | 70.441 | −6.709 | 1.00 | 0.00 | H |
| ATOM | 647 | N | HIS | A | 209 | 33.066 | 67.136 | −1.188 | 1.00 | 0.00 | N |
| ATOM | 648 | CA | HIS | A | 209 | 33.197 | 67.389 | 0.238 | 1.00 | 0.00 | C |
| ATOM | 649 | C | HIS | A | 209 | 32.878 | 66.119 | 1.022 | 1.00 | 0.00 | C |
| ATOM | 650 | O | HIS | A | 209 | 33.548 | 65.808 | 2.002 | 1.00 | 0.00 | O |
| ATOM | 651 | CB | HIS | A | 209 | 32.257 | 68.533 | 0.634 | 1.00 | 0.00 | C |
| ATOM | 652 | CG | HIS | A | 209 | 32.538 | 69.805 | −0.126 | 1.00 | 0.00 | C |
| ATOM | 653 | CD2 | HIS | A | 209 | 33.452 | 70.810 | 0.075 | 1.00 | 0.00 | C |
| ATOM | 654 | ND1 | HIS | A | 209 | 31.846 | 70.168 | −1.277 | 1.00 | 0.00 | N |
| ATOM | 655 | CE1 | HIS | A | 209 | 32.354 | 71.328 | −1.720 | 1.00 | 0.00 | C |
| ATOM | 656 | NE2 | HIS | A | 209 | 33.362 | 71.738 | −0.953 | 1.00 | 0.00 | N |
| ATOM | 657 | H | HIS | A | 209 | 32.124 | 67.013 | −1.532 | 1.00 | 0.00 | H |
| ATOM | 658 | HA | HIS | A | 209 | 34.207 | 67.738 | 0.454 | 1.00 | 0.00 | H |
| ATOM | 659 | 1HB | HIS | A | 209 | 31.226 | 68.244 | 0.431 | 1.00 | 0.00 | H |
| ATOM | 660 | 2HB | HIS | A | 209 | 32.371 | 68.746 | 1.697 | 1.00 | 0.00 | H |
| ATOM | 661 | HD2 | HIS | A | 209 | 34.100 | 70.786 | 0.939 | 1.00 | 0.00 | H |
| ATOM | 662 | HD1 | HIS | A | 209 | 31.103 | 69.579 | −1.626 | 1.00 | 0.00 | H |
| ATOM | 663 | HE1 | HIS | A | 209 | 31.937 | 71.801 | −2.597 | 1.00 | 0.00 | H |
| ATOM | 664 | N | ALA | A | 210 | 31.858 | 65.374 | 0.601 | 1.00 | 0.00 | N |
| ATOM | 665 | CA | ALA | A | 210 | 31.453 | 64.164 | 1.292 | 1.00 | 0.00 | C |
| ATOM | 666 | C | ALA | A | 210 | 32.590 | 63.143 | 1.287 | 1.00 | 0.00 | C |
| ATOM | 667 | O | ALA | A | 210 | 32.787 | 62.439 | 2.272 | 1.00 | 0.00 | O |
| ATOM | 668 | CB | ALA | A | 210 | 30.194 | 63.585 | 0.643 | 1.00 | 0.00 | C |
| ATOM | 669 | H | ALA | A | 210 | 31.341 | 65.645 | −0.223 | 1.00 | 0.00 | H |
| ATOM | 670 | HA | ALA | A | 210 | 31.148 | 64.410 | 2.309 | 1.00 | 0.00 | H |
| ATOM | 671 | 1HB | ALA | A | 210 | 29.898 | 62.677 | 1.168 | 1.00 | 0.00 | H |
| ATOM | 672 | 2HB | ALA | A | 210 | 29.388 | 64.316 | 0.698 | 1.00 | 0.00 | H |
| ATOM | 673 | 3HB | ALA | A | 210 | 30.399 | 63.349 | −0.402 | 1.00 | 0.00 | H |
| ATOM | 674 | N | ILE | A | 211 | 33.312 | 63.034 | 0.169 | 1.00 | 0.00 | N |
| ATOM | 675 | CA | ILE | A | 211 | 34.407 | 62.086 | 0.060 | 1.00 | 0.00 | C |
| ATOM | 676 | C | ILE | A | 211 | 35.436 | 62.326 | 1.181 | 1.00 | 0.00 | C |
| ATOM | 677 | O | ILE | A | 211 | 35.715 | 61.398 | 1.940 | 1.00 | 0.00 | O |
| ATOM | 678 | CB | ILE | A | 211 | 34.963 | 62.025 | −1.381 | 1.00 | 0.00 | C |
| ATOM | 679 | CG1 | ILE | A | 211 | 33.864 | 61.511 | −2.337 | 1.00 | 0.00 | C |
| ATOM | 680 | CG2 | ILE | A | 211 | 36.186 | 61.097 | −1.442 | 1.00 | 0.00 | C |
| ATOM | 681 | CD1 | ILE | A | 211 | 34.223 | 61.629 | −3.823 | 1.00 | 0.00 | C |
| ATOM | 682 | H | ILE | A | 211 | 33.101 | 63.618 | −0.627 | 1.00 | 0.00 | H |
| ATOM | 683 | HA | ILE | A | 211 | 34.007 | 61.078 | −0.052 | 1.00 | 0.00 | H |
| ATOM | 684 | HB | ILE | A | 211 | 35.294 | 63.018 | −1.686 | 1.00 | 0.00 | H |
| ATOM | 685 | 1HG1 | ILE | A | 211 | 33.672 | 60.456 | −2.137 | 1.00 | 0.00 | H |
| ATOM | 686 | 2HG1 | ILE | A | 211 | 32.949 | 62.082 | −2.182 | 1.00 | 0.00 | H |
| ATOM | 687 | 1HG2 | ILE | A | 211 | 36.567 | 61.063 | −2.462 | 1.00 | 0.00 | H |
| ATOM | 688 | 2HG2 | ILE | A | 211 | 36.963 | 61.474 | −0.777 | 1.00 | 0.00 | H |
| ATOM | 689 | 3HG2 | ILE | A | 211 | 35.897 | 60.093 | −1.129 | 1.00 | 0.00 | H |
| ATOM | 690 | 1HD1 | ILE | A | 211 | 33.400 | 61.247 | −4.427 | 1.00 | 0.00 | H |
| ATOM | 691 | 2HD1 | ILE | A | 211 | 34.400 | 62.676 | −4.072 | 1.00 | 0.00 | H |
| ATOM | 692 | 3HD1 | ILE | A | 211 | 35.123 | 61.050 | −4.027 | 1.00 | 0.00 | H |
| ATOM | 693 | N | PRO | A | 212 | 36.004 | 63.532 | 1.332 | 1.00 | 0.00 | N |
| ATOM | 694 | CA | PRO | A | 212 | 37.029 | 63.752 | 2.339 | 1.00 | 0.00 | C |
| ATOM | 695 | C | PRO | A | 212 | 36.454 | 63.715 | 3.758 | 1.00 | 0.00 | C |
| ATOM | 696 | O | PRO | A | 212 | 37.130 | 63.261 | 4.678 | 1.00 | 0.00 | O |
| ATOM | 697 | CB | PRO | A | 212 | 37.694 | 65.092 | 2.012 | 1.00 | 0.00 | C |
| ATOM | 698 | CG | PRO | A | 212 | 36.707 | 65.772 | 1.065 | 1.00 | 0.00 | C |
| ATOM | 699 | CD | PRO | A | 212 | 36.033 | 64.602 | 0.355 | 1.00 | 0.00 | C |
| ATOM | 700 | HA | PRO | A | 212 | 37.822 | 63.013 | 2.219 | 1.00 | 0.00 | H |
| ATOM | 701 | 1HB | PRO | A | 212 | 37.838 | 65.661 | 2.931 | 1.00 | 0.00 | H |
| ATOM | 702 | 2HB | PRO | A | 212 | 38.660 | 64.914 | 1.540 | 1.00 | 0.00 | H |
| ATOM | 703 | 1HG | PRO | A | 212 | 36.001 | 66.369 | 1.642 | 1.00 | 0.00 | H |
| ATOM | 704 | 2HG | PRO | A | 212 | 37.250 | 66.418 | 0.376 | 1.00 | 0.00 | H |
| ATOM | 705 | 1HD | PRO | A | 212 | 36.602 | 64.281 | −0.518 | 1.00 | 0.00 | H |
| ATOM | 706 | 2HD | PRO | A | 212 | 35.015 | 64.848 | 0.055 | 1.00 | 0.00 | H |
| ATOM | 707 | N | ILE | A | 213 | 35.212 | 64.165 | 3.955 | 1.00 | 0.00 | N |
| ATOM | 708 | CA | ILE | A | 213 | 34.601 | 64.106 | 5.278 | 1.00 | 0.00 | C |
| ATOM | 709 | C | ILE | A | 213 | 34.507 | 62.645 | 5.724 | 1.00 | 0.00 | C |
| ATOM | 710 | O | ILE | A | 213 | 34.860 | 62.315 | 6.854 | 1.00 | 0.00 | O |
| ATOM | 711 | CB | ILE | A | 213 | 33.225 | 64.800 | 5.302 | 1.00 | 0.00 | C |
| ATOM | 712 | CG1 | ILE | A | 213 | 33.372 | 66.316 | 5.082 | 1.00 | 0.00 | C |
| ATOM | 713 | CG2 | ILE | A | 213 | 32.550 | 64.553 | 6.662 | 1.00 | 0.00 | C |
| ATOM | 714 | CD1 | ILE | A | 213 | 32.060 | 66.963 | 4.622 | 1.00 | 0.00 | C |
| ATOM | 715 | H | ILE | A | 213 | 34.687 | 64.551 | 3.184 | 1.00 | 0.00 | H |
| ATOM | 716 | HA | ILE | A | 213 | 35.170 | 64.726 | 5.970 | 1.00 | 0.00 | H |
| ATOM | 717 | HB | ILE | A | 213 | 32.595 | 64.386 | 4.515 | 1.00 | 0.00 | H |
| ATOM | 718 | 1HG1 | ILE | A | 213 | 33.675 | 66.791 | 6.015 | 1.00 | 0.00 | H |
| ATOM | 719 | 2HG1 | ILE | A | 213 | 34.127 | 66.502 | 4.319 | 1.00 | 0.00 | H |
| ATOM | 720 | 1HG2 | ILE | A | 213 | 31.577 | 65.043 | 6.679 | 1.00 | 0.00 | H |
| ATOM | 721 | 2HG2 | ILE | A | 213 | 32.419 | 63.481 | 6.814 | 1.00 | 0.00 | H |
| ATOM | 722 | 3HG2 | ILE | A | 213 | 33.176 | 64.958 | 7.457 | 1.00 | 0.00 | H |
| ATOM | 723 | 1HD1 | ILE | A | 213 | 32.213 | 68.033 | 4.480 | 1.00 | 0.00 | H |
| ATOM | 724 | 2HD1 | ILE | A | 213 | 31.743 | 66.514 | 3.681 | 1.00 | 0.00 | H |
| ATOM | 725 | 3HD1 | ILE | A | 213 | 31.291 | 66.804 | 5.378 | 1.00 | 0.00 | H |
| ATOM | 726 | N | ILE | A | 214 | 34.020 | 61.771 | 4.844 | 1.00 | 0.00 | N |
| ATOM | 727 | CA | ILE | A | 214 | 33.851 | 60.368 | 5.181 | 1.00 | 0.00 | C |
| ATOM | 728 | C | ILE | A | 214 | 35.234 | 59.729 | 5.325 | 1.00 | 0.00 | C |
| ATOM | 729 | O | ILE | A | 214 | 35.434 | 58.791 | 6.103 | 1.00 | 0.00 | O |
| ATOM | 730 | CB | ILE | A | 214 | 32.978 | 59.682 | 4.111 | 1.00 | 0.00 | C |
| ATOM | 731 | CG1 | ILE | A | 214 | 31.559 | 60.283 | 4.157 | 1.00 | 0.00 | C |
| ATOM | 732 | CG2 | ILE | A | 214 | 32.914 | 58.160 | 4.318 | 1.00 | 0.00 | C |
| ATOM | 733 | CD1 | ILE | A | 214 | 30.701 | 59.905 | 2.944 | 1.00 | 0.00 | C |
| ATOM | 734 | H | ILE | A | 214 | 33.759 | 62.079 | 3.918 | 1.00 | 0.00 | H |
| ATOM | 735 | HA | ILE | A | 214 | 33.245 | 60.281 | 6.083 | 1.00 | 0.00 | H |
| ATOM | 736 | HB | ILE | A | 214 | 33.432 | 59.816 | 3.130 | 1.00 | 0.00 | H |
| ATOM | 737 | 1HG1 | ILE | A | 214 | 31.041 | 59.927 | 5.047 | 1.00 | 0.00 | H |
| ATOM | 738 | 2HG1 | ILE | A | 214 | 31.625 | 61.370 | 4.185 | 1.00 | 0.00 | H |
| ATOM | 739 | 1HG2 | ILE | A | 214 | 32.289 | 57.713 | 3.545 | 1.00 | 0.00 | H |
| ATOM | 740 | 2HG2 | ILE | A | 214 | 33.919 | 57.742 | 4.259 | 1.00 | 0.00 | H |
| ATOM | 741 | 3HG2 | ILE | A | 214 | 32.489 | 57.943 | 5.298 | 1.00 | 0.00 | H |
| ATOM | 742 | 1HD1 | ILE | A | 214 | 29.715 | 60.361 | 3.040 | 1.00 | 0.00 | H |
| ATOM | 743 | 2HD1 | ILE | A | 214 | 31.179 | 60.265 | 2.033 | 1.00 | 0.00 | H |
| ATOM | 744 | 3HD1 | ILE | A | 214 | 30.596 | 58.822 | 2.896 | 1.00 | 0.00 | H |
| ATOM | 745 | N | LYS | A | 215 | 36.198 | 60.215 | 4.544 | 1.00 | 0.00 | N |
| ATOM | 746 | CA | LYS | A | 215 | 37.553 | 59.729 | 4.629 | 1.00 | 0.00 | C |
| ATOM | 747 | C | LYS | A | 215 | 38.141 | 60.049 | 6.007 | 1.00 | 0.00 | C |
| ATOM | 748 | O | LYS | A | 215 | 38.822 | 59.206 | 6.602 | 1.00 | 0.00 | O |
| ATOM | 749 | CB | LYS | A | 215 | 38.394 | 60.304 | 3.483 | 1.00 | 0.00 | C |
| ATOM | 750 | CG | LYS | A | 215 | 39.829 | 59.776 | 3.465 | 1.00 | 0.00 | C |
| ATOM | 751 | CD | LYS | A | 215 | 39.908 | 58.273 | 3.177 | 1.00 | 0.00 | C |
| ATOM | 752 | CE | LYS | A | 215 | 41.270 | 57.754 | 3.628 | 1.00 | 0.00 | C |
| ATOM | 753 | NZ | LYS | A | 215 | 41.382 | 56.304 | 3.400 | 1.00 | 0.00 | N1+ |
| ATOM | 754 | H | LYS | A | 215 | 35.988 | 60.940 | 3.874 | 1.00 | 0.00 | H |
| ATOM | 755 | HA | LYS | A | 215 | 37.573 | 58.664 | 4.396 | 1.00 | 0.00 | H |
| ATOM | 756 | 1HB | LYS | A | 215 | 37.936 | 60.043 | 2.529 | 1.00 | 0.00 | H |
| ATOM | 757 | 2HB | LYS | A | 215 | 38.444 | 61.389 | 3.577 | 1.00 | 0.00 | H |
| ATOM | 758 | 1HG | LYS | A | 215 | 40.397 | 60.292 | 2.691 | 1.00 | 0.00 | H |
| ATOM | 759 | 2HG | LYS | A | 215 | 40.294 | 59.953 | 4.435 | 1.00 | 0.00 | H |
| ATOM | 760 | 1HD | LYS | A | 215 | 39.118 | 57.756 | 3.723 | 1.00 | 0.00 | H |
| ATOM | 761 | 2HD | LYS | A | 215 | 39.783 | 58.100 | 2.108 | 1.00 | 0.00 | H |
| ATOM | 762 | 1HE | LYS | A | 215 | 42.056 | 58.258 | 3.065 | 1.00 | 0.00 | H |
| ATOM | 763 | 2HE | LYS | A | 215 | 41.401 | 57.952 | 4.691 | 1.00 | 0.00 | H |
| ATOM | 764 | 1HZ | LYS | A | 215 | 42.290 | 55.984 | 3.705 | 1.00 | 0.00 | H |
| ATOM | 765 | 2HZ | LYS | A | 215 | 40.663 | 55.824 | 3.923 | 1.00 | 0.00 | H |
| ATOM | 766 | 3HZ | LYS | A | 215 | 41.270 | 56.107 | 2.416 | 1.00 | 0.00 | H |
| ATOM | 767 | N | GLU | A | 216 | 37.876 | 61.252 | 6.515 | 1.00 | 0.00 | N |
| ATOM | 768 | CA | GLU | A | 216 | 38.435 | 61.758 | 7.755 | 1.00 | 0.00 | C |
| ATOM | 769 | C | GLU | A | 216 | 37.367 | 61.890 | 8.839 | 1.00 | 0.00 | C |
| ATOM | 770 | O | GLU | A | 216 | 37.433 | 62.761 | 9.702 | 1.00 | 0.00 | O |
| ATOM | 771 | CB | GLU | A | 216 | 39.290 | 63.007 | 7.501 | 1.00 | 0.00 | C |
| ATOM | 772 | CG | GLU | A | 216 | 40.502 | 62.681 | 6.602 | 1.00 | 0.00 | C |
| ATOM | 773 | CD | GLU | A | 216 | 41.312 | 61.515 | 7.148 | 1.00 | 0.00 | C |
| ATOM | 774 | OE1 | GLU | A | 216 | 41.524 | 61.472 | 8.379 | 1.00 | 0.00 | O |
| ATOM | 775 | OE2 | GLU | A | 216 | 41.550 | 60.537 | 6.408 | 1.00 | 0.00 | O1− |
| ATOM | 776 | H | GLU | A | 216 | 37.250 | 61.878 | 6.029 | 1.00 | 0.00 | H |
| ATOM | 777 | HA | GLU | A | 216 | 39.327 | 61.187 | 8.012 | 1.00 | 0.00 | H |
| ATOM | 778 | 1HB | GLU | A | 216 | 38.685 | 63.767 | 7.007 | 1.00 | 0.00 | H |
| ATOM | 779 | 2HB | GLU | A | 216 | 39.657 | 63.396 | 8.451 | 1.00 | 0.00 | H |
| ATOM | 780 | 1HG | GLU | A | 216 | 40.154 | 62.417 | 5.603 | 1.00 | 0.00 | H |
| ATOM | 781 | 2HG | GLU | A | 216 | 41.155 | 63.551 | 6.541 | 1.00 | 0.00 | H |
| ATOM | 782 | N | LYS | A | 217 | 36.498 | 60.878 | 8.884 | 1.00 | 0.00 | N |
| ATOM | 783 | CA | LYS | A | 217 | 36.071 | 60.255 | 10.127 | 1.00 | 0.00 | C |
| ATOM | 784 | C | LYS | A | 217 | 35.284 | 61.217 | 11.024 | 1.00 | 0.00 | C |
| ATOM | 785 | O | LYS | A | 217 | 35.485 | 61.232 | 12.237 | 1.00 | 0.00 | O |
| ATOM | 786 | CB | LYS | A | 217 | 37.304 | 59.673 | 10.855 | 1.00 | 0.00 | C |
| ATOM | 787 | CG | LYS | A | 217 | 38.153 | 58.725 | 9.981 | 1.00 | 0.00 | C |
| ATOM | 788 | CD | LYS | A | 217 | 39.587 | 58.599 | 10.527 | 1.00 | 0.00 | C |
| ATOM | 789 | CE | LYS | A | 217 | 40.573 | 58.009 | 9.502 | 1.00 | 0.00 | C |
| ATOM | 790 | NZ | LYS | A | 217 | 40.866 | 58.949 | 8.400 | 1.00 | 0.00 | N1+ |
| ATOM | 791 | H | LYS | A | 217 | 36.105 | 60.511 | 8.029 | 1.00 | 0.00 | H |
| ATOM | 792 | HA | LYS | A | 217 | 35.499 | 59.354 | 9.905 | 1.00 | 0.00 | H |
| ATOM | 793 | 1HB | LYS | A | 217 | 37.954 | 60.486 | 11.177 | 1.00 | 0.00 | H |
| ATOM | 794 | 2HB | LYS | A | 217 | 36.977 | 59.104 | 11.726 | 1.00 | 0.00 | H |
| ATOM | 795 | 1HG | LYS | A | 217 | 37.697 | 57.735 | 9.969 | 1.00 | 0.00 | H |
| ATOM | 796 | 2HG | LYS | A | 217 | 38.202 | 59.114 | 8.964 | 1.00 | 0.00 | H |
| ATOM | 797 | 1HD | LYS | A | 217 | 39.956 | 59.584 | 10.812 | 1.00 | 0.00 | H |
| ATOM | 798 | 2HD | LYS | A | 217 | 39.588 | 57.945 | 11.400 | 1.00 | 0.00 | H |
| ATOM | 799 | 1HE | LYS | A | 217 | 41.514 | 57.768 | 9.998 | 1.00 | 0.00 | H |
| ATOM | 800 | 2HE | LYS | A | 217 | 40.149 | 57.103 | 9.068 | 1.00 | 0.00 | H |
| ATOM | 801 | 1HZ | LYS | A | 217 | 41.514 | 58.521 | 7.755 | 1.00 | 0.00 | H |
| ATOM | 802 | 2HZ | LYS | A | 217 | 40.010 | 59.177 | 7.915 | 1.00 | 0.00 | H |
| ATOM | 803 | 3HZ | LYS | A | 217 | 41.273 | 59.793 | 8.776 | 1.00 | 0.00 | H |
| ATOM | 804 | N | ARG | A | 218 | 34.359 | 61.971 | 10.435 | 1.00 | 0.00 | N |
| ATOM | 805 | CA | ARG | A | 218 | 33.481 | 62.886 | 11.147 | 1.00 | 0.00 | C |
| ATOM | 806 | C | ARG | A | 218 | 32.060 | 62.589 | 10.684 | 1.00 | 0.00 | C |
| ATOM | 807 | O | ARG | A | 218 | 31.800 | 62.611 | 9.482 | 1.00 | 0.00 | O |
| ATOM | 808 | CB | ARG | A | 218 | 33.851 | 64.331 | 10.795 | 1.00 | 0.00 | C |
| ATOM | 809 | CG | ARG | A | 218 | 35.202 | 64.771 | 11.381 | 1.00 | 0.00 | C |
| ATOM | 810 | CD | ARG | A | 218 | 35.125 | 65.172 | 12.864 | 1.00 | 0.00 | C |
| ATOM | 811 | NE | ARG | A | 218 | 34.131 | 66.227 | 13.125 | 1.00 | 0.00 | N |
| ATOM | 812 | CZ | ARG | A | 218 | 34.188 | 67.503 | 12.729 | 1.00 | 0.00 | C |
| ATOM | 813 | NH1 | ARG | A | 218 | 35.228 | 67.927 | 12.011 | 1.00 | 0.00 | N1+ |
| ATOM | 814 | NH2 | ARG | A | 218 | 33.200 | 68.345 | 12.995 | 1.00 | 0.00 | N |
| ATOM | 815 | H | ARG | A | 218 | 34.235 | 61.927 | 9.434 | 1.00 | 0.00 | H |
| ATOM | 816 | HA | ARG | A | 218 | 33.565 | 62.710 | 12.220 | 1.00 | 0.00 | H |
| ATOM | 817 | 1HB | ARG | A | 218 | 33.912 | 64.438 | 9.712 | 1.00 | 0.00 | H |
| ATOM | 818 | 2HB | ARG | A | 218 | 33.089 | 65.007 | 11.184 | 1.00 | 0.00 | H |
| ATOM | 819 | 1HG | ARG | A | 218 | 35.917 | 63.953 | 11.301 | 1.00 | 0.00 | H |
| ATOM | 820 | 2HG | ARG | A | 218 | 35.574 | 65.634 | 10.829 | 1.00 | 0.00 | H |
| ATOM | 821 | 1HD | ARG | A | 218 | 34.850 | 64.303 | 13.462 | 1.00 | 0.00 | H |
| ATOM | 822 | 2HD | ARG | A | 218 | 36.096 | 65.544 | 13.191 | 1.00 | 0.00 | H |
| ATOM | 823 | HE | ARG | A | 218 | 33.342 | 65.901 | 13.665 | 1.00 | 0.00 | H |
| ATOM | 824 | 1HH1 | ARG | A | 218 | 35.274 | 68.890 | 11.709 | 1.00 | 0.00 | H |
| ATOM | 825 | 2HH1 | ARG | A | 218 | 35.969 | 67.285 | 11.768 | 1.00 | 0.00 | H |
| ATOM | 826 | 1HH2 | ARG | A | 218 | 33.258 | 69.306 | 12.688 | 1.00 | 0.00 | H |
| ATOM | 827 | 2HH2 | ARG | A | 218 | 32.388 | 68.027 | 13.505 | 1.00 | 0.00 | H |
| ATOM | 828 | N | ASP | A | 219 | 31.163 | 62.301 | 11.628 | 1.00 | 0.00 | N |
| ATOM | 829 | CA | ASP | A | 219 | 29.793 | 61.909 | 11.335 | 1.00 | 0.00 | C |
| ATOM | 830 | C | ASP | A | 219 | 29.087 | 63.039 | 10.577 | 1.00 | 0.00 | C |
| ATOM | 831 | O | ASP | A | 219 | 29.419 | 64.217 | 10.735 | 1.00 | 0.00 | O |
| ATOM | 832 | CB | ASP | A | 219 | 29.075 | 61.415 | 12.608 | 1.00 | 0.00 | C |
| ATOM | 833 | CG | ASP | A | 219 | 29.380 | 59.951 | 12.922 | 1.00 | 0.00 | C |
| ATOM | 834 | OD1 | ASP | A | 219 | 30.307 | 59.393 | 12.299 | 1.00 | 0.00 | O |
| ATOM | 835 | OD2 | ASP | A | 219 | 28.643 | 59.327 | 13.715 | 1.00 | 0.00 | O1− |
| ATOM | 836 | H | ASP | A | 219 | 31.423 | 62.350 | 12.603 | 1.00 | 0.00 | H |
| ATOM | 837 | HA | ASP | A | 219 | 29.789 | 60.930 | 10.855 | 1.00 | 0.00 | H |
| ATOM | 838 | 1HB | ASP | A | 219 | 29.393 | 62.014 | 13.461 | 1.00 | 0.00 | H |
| ATOM | 839 | 2HB | ASP | A | 219 | 27.997 | 61.512 | 12.478 | 1.00 | 0.00 | H |
| ATOM | 840 | N | LEU | A | 220 | 28.168 | 62.679 | 9.682 | 1.00 | 0.00 | N |
| ATOM | 841 | CA | LEU | A | 220 | 27.842 | 63.509 | 8.531 | 1.00 | 0.00 | C |
| ATOM | 842 | C | LEU | A | 220 | 26.334 | 63.543 | 8.301 | 1.00 | 0.00 | C |
| ATOM | 843 | O | LEU | A | 220 | 25.679 | 62.502 | 8.282 | 1.00 | 0.00 | O |
| ATOM | 844 | CB | LEU | A | 220 | 28.568 | 62.932 | 7.303 | 1.00 | 0.00 | C |
| ATOM | 845 | CG | LEU | A | 220 | 28.181 | 63.556 | 5.948 | 1.00 | 0.00 | C |
| ATOM | 846 | CD1 | LEU | A | 220 | 28.521 | 65.049 | 5.869 | 1.00 | 0.00 | C |
| ATOM | 847 | CD2 | LEU | A | 220 | 28.932 | 62.824 | 4.830 | 1.00 | 0.00 | C |
| ATOM | 848 | H | LEU | A | 220 | 27.672 | 61.806 | 9.791 | 1.00 | 0.00 | H |
| ATOM | 849 | HA | LEU | A | 220 | 28.218 | 64.519 | 8.693 | 1.00 | 0.00 | H |
| ATOM | 850 | 1HB | LEU | A | 220 | 29.643 | 63.080 | 7.413 | 1.00 | 0.00 | H |
| ATOM | 851 | 2HB | LEU | A | 220 | 28.355 | 61.866 | 7.222 | 1.00 | 0.00 | H |
| ATOM | 852 | HG | LEU | A | 220 | 27.109 | 63.440 | 5.787 | 1.00 | 0.00 | H |
| ATOM | 853 | 1HD1 | LEU | A | 220 | 28.228 | 65.438 | 4.894 | 1.00 | 0.00 | H |
| ATOM | 854 | 2HD1 | LEU | A | 220 | 27.984 | 65.586 | 6.651 | 1.00 | 0.00 | H |
| ATOM | 855 | 3HD1 | LEU | A | 220 | 29.594 | 65.185 | 6.006 | 1.00 | 0.00 | H |
| ATOM | 856 | 1HD2 | LEU | A | 220 | 28.664 | 63.258 | 3.867 | 1.00 | 0.00 | H |
| ATOM | 857 | 2HD2 | LEU | A | 220 | 30.006 | 62.923 | 4.988 | 1.00 | 0.00 | H |
| ATOM | 858 | 3HD2 | LEU | A | 220 | 28.660 | 61.768 | 4.841 | 1.00 | 0.00 | H |
| ATOM | 859 | N | MET | A | 221 | 25.811 | 64.741 | 8.040 | 1.00 | 0.00 | N |
| ATOM | 860 | CA | MET | A | 221 | 24.512 | 64.949 | 7.428 | 1.00 | 0.00 | C |
| ATOM | 861 | C | MET | A | 221 | 24.793 | 65.579 | 6.068 | 1.00 | 0.00 | C |
| ATOM | 862 | O | MET | A | 221 | 25.418 | 66.636 | 6.016 | 1.00 | 0.00 | O |
| ATOM | 863 | CB | MET | A | 221 | 23.701 | 65.911 | 8.302 | 1.00 | 0.00 | C |
| ATOM | 864 | CG | MET | A | 221 | 22.294 | 66.179 | 7.764 | 1.00 | 0.00 | C |
| ATOM | 865 | SD | MET | A | 221 | 21.200 | 64.741 | 7.836 | 1.00 | 0.00 | S |
| ATOM | 866 | CE | MET | A | 221 | 20.778 | 64.575 | 6.086 | 1.00 | 0.00 | C |
| ATOM | 867 | H | MET | A | 221 | 26.327 | 65.578 | 8.272 | 1.00 | 0.00 | H |
| ATOM | 868 | HA | MET | A | 221 | 24.013 | 63.988 | 7.298 | 1.00 | 0.00 | H |
| ATOM | 869 | 1HB | MET | A | 221 | 23.594 | 65.492 | 9.303 | 1.00 | 0.00 | H |
| ATOM | 870 | 2HB | MET | A | 221 | 24.216 | 66.870 | 8.363 | 1.00 | 0.00 | H |
| ATOM | 871 | 1HG | MET | A | 221 | 21.826 | 66.971 | 8.348 | 1.00 | 0.00 | H |
| ATOM | 872 | 2HG | MET | A | 221 | 22.356 | 66.486 | 6.720 | 1.00 | 0.00 | H |
| ATOM | 873 | 1HE | MET | A | 221 | 20.104 | 63.728 | 5.953 | 1.00 | 0.00 | H |
| ATOM | 874 | 2HE | MET | A | 221 | 20.288 | 65.486 | 5.741 | 1.00 | 0.00 | H |
| ATOM | 875 | 3HE | MET | A | 221 | 21.686 | 64.410 | 5.506 | 1.00 | 0.00 | H |
| ATOM | 876 | N | ALA | A | 222 | 24.347 | 64.959 | 4.980 | 1.00 | 0.00 | N |
| ATOM | 877 | CA | ALA | A | 222 | 24.584 | 65.429 | 3.627 | 1.00 | 0.00 | C |
| ATOM | 878 | C | ALA | A | 222 | 23.273 | 65.385 | 2.846 | 1.00 | 0.00 | C |
| ATOM | 879 | O | ALA | A | 222 | 22.987 | 64.419 | 2.143 | 1.00 | 0.00 | O |
| ATOM | 880 | CB | ALA | A | 222 | 25.652 | 64.546 | 2.983 | 1.00 | 0.00 | C |
| ATOM | 881 | H | ALA | A | 222 | 23.809 | 64.108 | 5.068 | 1.00 | 0.00 | H |
| ATOM | 882 | HA | ALA | A | 222 | 24.990 | 66.440 | 3.659 | 1.00 | 0.00 | H |
| ATOM | 883 | 1HB | ALA | A | 222 | 25.841 | 64.887 | 1.965 | 1.00 | 0.00 | H |
| ATOM | 884 | 2HB | ALA | A | 222 | 26.573 | 64.607 | 3.563 | 1.00 | 0.00 | H |
| ATOM | 885 | 3HB | ALA | A | 222 | 25.306 | 63.513 | 2.960 | 1.00 | 0.00 | H |
| ATOM | 886 | N | CYS | A | 223 | 22.484 | 66.452 | 2.939 | 1.00 | 0.00 | N |
| ATOM | 887 | CA | CYS | A | 223 | 21.190 | 66.511 | 2.278 | 1.00 | 0.00 | C |
| ATOM | 888 | C | CYS | A | 223 | 21.400 | 66.872 | 0.806 | 1.00 | 0.00 | C |
| ATOM | 889 | O | CYS | A | 223 | 22.098 | 67.839 | 0.503 | 1.00 | 0.00 | O |
| ATOM | 890 | CB | CYS | A | 223 | 20.293 | 67.531 | 2.991 | 1.00 | 0.00 | C |
| ATOM | 891 | SG | CYS | A | 223 | 18.814 | 67.867 | 2.002 | 1.00 | 0.00 | S |
| ATOM | 892 | H | CYS | A | 223 | 22.778 | 67.253 | 3.479 | 1.00 | 0.00 | H |
| ATOM | 893 | HA | CYS | A | 223 | 20.680 | 65.555 | 2.392 | 1.00 | 0.00 | H |
| ATOM | 894 | 1HB | CYS | A | 223 | 19.991 | 67.136 | 3.960 | 1.00 | 0.00 | H |
| ATOM | 895 | 2HB | CYS | A | 223 | 20.843 | 68.462 | 3.135 | 1.00 | 0.00 | H |
| ATOM | 896 | HG | CYS | A | 223 | 18.181 | 68.600 | 2.524 | 1.00 | 0.00 | H |
| ATOM | 897 | N | ALA | A | 224 | 20.787 | 66.149 | −0.130 | 1.00 | 0.00 | N |
| ATOM | 898 | CA | ALA | A | 224 | 20.885 | 66.493 | −1.538 | 1.00 | 0.00 | C |
| ATOM | 899 | C | ALA | A | 224 | 19.762 | 65.830 | −2.329 | 1.00 | 0.00 | C |
| ATOM | 900 | O | ALA | A | 224 | 19.564 | 64.622 | −2.231 | 1.00 | 0.00 | O |
| ATOM | 901 | CB | ALA | A | 224 | 22.248 | 66.060 | −2.090 | 1.00 | 0.00 | C |
| ATOM | 902 | H | ALA | A | 224 | 20.239 | 65.343 | 0.134 | 1.00 | 0.00 | H |
| ATOM | 903 | HA | ALA | A | 224 | 20.842 | 67.576 | −1.652 | 1.00 | 0.00 | H |
| ATOM | 904 | 1HB | ALA | A | 224 | 22.313 | 66.322 | −3.146 | 1.00 | 0.00 | H |
| ATOM | 905 | 2HB | ALA | A | 224 | 23.041 | 66.568 | −1.541 | 1.00 | 0.00 | H |
| ATOM | 906 | 3HB | ALA | A | 224 | 22.361 | 64.982 | −1.976 | 1.00 | 0.00 | H |
| ATOM | 907 | N | GLN | A | 225 | 19.052 | 66.602 | −3.148 | 1.00 | 0.00 | N |
| ATOM | 908 | CA | GLN | A | 225 | 18.011 | 66.070 | −4.016 | 1.00 | 0.00 | C |
| ATOM | 909 | C | GLN | A | 225 | 18.551 | 64.940 | −4.907 | 1.00 | 0.00 | C |
| ATOM | 910 | O | GLN | A | 225 | 19.741 | 64.912 | −5.232 | 1.00 | 0.00 | O |
| ATOM | 911 | CB | GLN | A | 225 | 17.413 | 67.202 | −4.863 | 1.00 | 0.00 | C |
| ATOM | 912 | CG | GLN | A | 225 | 18.470 | 68.003 | −5.638 | 1.00 | 0.00 | C |
| ATOM | 913 | CD | GLN | A | 225 | 17.818 | 68.910 | −6.670 | 1.00 | 0.00 | C |
| ATOM | 914 | NE2 | GLN | A | 225 | 17.664 | 70.190 | −6.358 | 1.00 | 0.00 | N |
| ATOM | 915 | OE1 | GLN | A | 225 | 17.449 | 68.463 | −7.751 | 1.00 | 0.00 | O |
| ATOM | 916 | H | GLN | A | 225 | 19.227 | 67.596 | −3.183 | 1.00 | 0.00 | H |
| ATOM | 917 | HA | GLN | A | 225 | 17.172 | 65.728 | −3.410 | 1.00 | 0.00 | H |
| ATOM | 918 | 1HB | GLN | A | 225 | 16.719 | 66.782 | −5.592 | 1.00 | 0.00 | H |
| ATOM | 919 | 2HB | GLN | A | 225 | 16.881 | 67.899 | −4.216 | 1.00 | 0.00 | H |
| ATOM | 920 | 1HG | GLN | A | 225 | 19.042 | 68.618 | −4.943 | 1.00 | 0.00 | H |
| ATOM | 921 | 2HG | GLN | A | 225 | 19.142 | 67.316 | −6.152 | 1.00 | 0.00 | H |
| ATOM | 922 | 1HE2 | GLN | A | 225 | 17.236 | 70.823 | −7.018 | 1.00 | 0.00 | H |
| ATOM | 923 | 2HE2 | GLN | A | 225 | 17.976 | 70.532 | −5.460 | 1.00 | 0.00 | H |
| ATOM | 924 | N | THR | A | 226 | 17.683 | 64.025 | −5.352 | 1.00 | 0.00 | N |
| ATOM | 925 | CA | THR | A | 226 | 18.044 | 63.049 | −6.365 | 1.00 | 0.00 | C |
| ATOM | 926 | C | THR | A | 226 | 18.554 | 63.810 | −7.602 | 1.00 | 0.00 | C |
| ATOM | 927 | O | THR | A | 226 | 17.775 | 64.430 | −8.323 | 1.00 | 0.00 | O |
| ATOM | 928 | CB | THR | A | 226 | 16.816 | 62.179 | −6.698 | 1.00 | 0.00 | C |
| ATOM | 929 | CG2 | THR | A | 226 | 17.227 | 60.752 | −7.064 | 1.00 | 0.00 | C |
| ATOM | 930 | OG1 | THR | A | 226 | 15.894 | 62.136 | −5.621 | 1.00 | 0.00 | O |
| ATOM | 931 | H | THR | A | 226 | 16.743 | 63.999 | −4.982 | 1.00 | 0.00 | H |
| ATOM | 932 | HA | THR | A | 226 | 18.802 | 62.374 | −5.967 | 1.00 | 0.00 | H |
| ATOM | 933 | HB | THR | A | 226 | 16.258 | 62.636 | −7.515 | 1.00 | 0.00 | H |
| ATOM | 934 | 1HG2 | THR | A | 226 | 16.337 | 60.165 | −7.293 | 1.00 | 0.00 | H |
| ATOM | 935 | 2HG2 | THR | A | 226 | 17.881 | 60.774 | −7.935 | 1.00 | 0.00 | H |
| ATOM | 936 | 3HG2 | THR | A | 226 | 17.755 | 60.299 | −6.225 | 1.00 | 0.00 | H |
| ATOM | 937 | HG1 | THR | A | 226 | 15.144 | 61.587 | −5.863 | 1.00 | 0.00 | H |
| ATOM | 938 | N | GLY | A | 227 | 19.865 | 63.807 | −7.822 | 1.00 | 0.00 | N |
| ATOM | 939 | CA | GLY | A | 227 | 20.512 | 64.935 | −8.462 | 1.00 | 0.00 | C |
| ATOM | 940 | C | GLY | A | 227 | 22.008 | 64.645 | −8.481 | 1.00 | 0.00 | C |
| ATOM | 941 | O | GLY | A | 227 | 22.442 | 63.739 | −9.200 | 1.00 | 0.00 | O |
| ATOM | 942 | H | GLY | A | 227 | 20.423 | 63.013 | −7.542 | 1.00 | 0.00 | H |
| ATOM | 943 | 1HA | GLY | A | 227 | 20.130 | 65.045 | −9.476 | 1.00 | 0.00 | H |
| ATOM | 944 | 2HA | GLY | A | 227 | 20.305 | 65.843 | −7.895 | 1.00 | 0.00 | H |
| ATOM | 945 | N | SER | A | 228 | 22.780 | 65.323 | −7.633 | 1.00 | 0.00 | N |
| ATOM | 946 | CA | SER | A | 228 | 24.234 | 65.300 | −7.633 | 1.00 | 0.00 | C |
| ATOM | 947 | C | SER | A | 228 | 24.863 | 63.984 | −7.130 | 1.00 | 0.00 | C |
| ATOM | 948 | O | SER | A | 228 | 25.941 | 64.007 | −6.538 | 1.00 | 0.00 | O |
| ATOM | 949 | CB | SER | A | 228 | 24.722 | 66.472 | −6.771 | 1.00 | 0.00 | C |
| ATOM | 950 | OG | SER | A | 228 | 24.226 | 67.710 | −7.238 | 1.00 | 0.00 | O |
| ATOM | 951 | H | SER | A | 228 | 22.360 | 65.907 | −6.924 | 1.00 | 0.00 | H |
| ATOM | 952 | HA | SER | A | 228 | 24.602 | 65.502 | −8.639 | 1.00 | 0.00 | H |
| ATOM | 953 | 1HB | SER | A | 228 | 24.383 | 66.334 | −5.744 | 1.00 | 0.00 | H |
| ATOM | 954 | 2HB | SER | A | 228 | 25.811 | 66.511 | −6.792 | 1.00 | 0.00 | H |
| ATOM | 955 | HG | SER | A | 228 | 24.551 | 68.418 | −6.677 | 1.00 | 0.00 | H |
| ATOM | 956 | N | GLY | A | 229 | 24.254 | 62.826 | −7.387 | 1.00 | 0.00 | N |
| ATOM | 957 | CA | GLY | A | 229 | 24.921 | 61.541 | −7.240 | 1.00 | 0.00 | C |
| ATOM | 958 | C | GLY | A | 229 | 25.317 | 61.222 | −5.796 | 1.00 | 0.00 | C |
| ATOM | 959 | O | GLY | A | 229 | 26.332 | 60.560 | −5.569 | 1.00 | 0.00 | O |
| ATOM | 960 | H | GLY | A | 229 | 23.293 | 62.819 | −7.697 | 1.00 | 0.00 | H |
| ATOM | 961 | 1HA | GLY | A | 229 | 24.257 | 60.746 | −7.580 | 1.00 | 0.00 | H |
| ATOM | 962 | 2HA | GLY | A | 229 | 25.832 | 61.533 | −7.839 | 1.00 | 0.00 | H |
| ATOM | 963 | N | LYS | A | 230 | 24.502 | 61.647 | −4.825 | 1.00 | 0.00 | N |
| ATOM | 964 | CA | LYS | A | 230 | 24.684 | 61.288 | −3.423 | 1.00 | 0.00 | C |
| ATOM | 965 | C | LYS | A | 230 | 24.821 | 59.772 | −3.269 | 1.00 | 0.00 | C |
| ATOM | 966 | O | LYS | A | 230 | 25.666 | 59.290 | −2.522 | 1.00 | 0.00 | O |
| ATOM | 967 | CB | LYS | A | 230 | 23.530 | 61.831 | −2.571 | 1.00 | 0.00 | C |
| ATOM | 968 | CG | LYS | A | 230 | 22.151 | 61.274 | −2.955 | 1.00 | 0.00 | C |
| ATOM | 969 | CD | LYS | A | 230 | 21.072 | 61.917 | −2.082 | 1.00 | 0.00 | C |
| ATOM | 970 | CE | LYS | A | 230 | 19.661 | 61.665 | −2.631 | 1.00 | 0.00 | C |
| ATOM | 971 | NZ | LYS | A | 230 | 19.369 | 60.230 | −2.811 | 1.00 | 0.00 | N1+ |
| ATOM | 972 | H | LYS | A | 230 | 23.718 | 62.243 | −5.049 | 1.00 | 0.00 | H |
| ATOM | 973 | HA | LYS | A | 230 | 25.549 | 61.817 | −3.022 | 1.00 | 0.00 | H |
| ATOM | 974 | 1HB | LYS | A | 230 | 23.699 | 61.575 | −1.525 | 1.00 | 0.00 | H |
| ATOM | 975 | 2HB | LYS | A | 230 | 23.479 | 62.915 | −2.676 | 1.00 | 0.00 | H |
| ATOM | 976 | 1HG | LYS | A | 230 | 21.947 | 61.498 | −4.003 | 1.00 | 0.00 | H |
| ATOM | 977 | 2HG | LYS | A | 230 | 22.139 | 60.195 | −2.806 | 1.00 | 0.00 | H |
| ATOM | 978 | 1HD | LYS | A | 230 | 21.125 | 61.503 | −1.076 | 1.00 | 0.00 | H |
| ATOM | 979 | 2HD | LYS | A | 230 | 21.233 | 62.994 | −2.040 | 1.00 | 0.00 | H |
| ATOM | 980 | 1HE | LYS | A | 230 | 18.924 | 62.071 | −1.939 | 1.00 | 0.00 | H |
| ATOM | 981 | 2HE | LYS | A | 230 | 19.555 | 62.151 | −3.601 | 1.00 | 0.00 | H |
| ATOM | 982 | 1HZ | LYS | A | 230 | 18.432 | 60.120 | −3.172 | 1.00 | 0.00 | H |
| ATOM | 983 | 2HZ | LYS | A | 230 | 20.030 | 59.830 | −3.462 | 1.00 | 0.00 | H |
| ATOM | 984 | 3HZ | LYS | A | 230 | 19.444 | 59.757 | −1.922 | 1.00 | 0.00 | H |
| ATOM | 985 | N | THR | A | 231 | 24.011 | 59.037 | −4.026 | 1.00 | 0.00 | N |
| ATOM | 986 | CA | THR | A | 231 | 23.815 | 57.602 | −3.941 | 1.00 | 0.00 | C |
| ATOM | 987 | C | THR | A | 231 | 25.078 | 56.820 | −4.348 | 1.00 | 0.00 | C |
| ATOM | 988 | O | THR | A | 231 | 25.094 | 55.592 | −4.295 | 1.00 | 0.00 | O |
| ATOM | 989 | CB | THR | A | 231 | 22.590 | 57.314 | −4.830 | 1.00 | 0.00 | C |
| ATOM | 990 | CG2 | THR | A | 231 | 22.007 | 55.908 | −4.678 | 1.00 | 0.00 | C |
| ATOM | 991 | OG1 | THR | A | 231 | 21.578 | 58.256 | −4.495 | 1.00 | 0.00 | O |
| ATOM | 992 | H | THR | A | 231 | 23.455 | 59.478 | −4.745 | 1.00 | 0.00 | H |
| ATOM | 993 | HA | THR | A | 231 | 23.492 | 57.337 | −2.934 | 1.00 | 0.00 | H |
| ATOM | 994 | HB | THR | A | 231 | 22.863 | 57.440 | −5.878 | 1.00 | 0.00 | H |
| ATOM | 995 | 1HG2 | THR | A | 231 | 21.149 | 55.796 | −5.341 | 1.00 | 0.00 | H |
| ATOM | 996 | 2HG2 | THR | A | 231 | 22.766 | 55.170 | −4.938 | 1.00 | 0.00 | H |
| ATOM | 997 | 3HG2 | THR | A | 231 | 21.690 | 55.756 | −3.647 | 1.00 | 0.00 | H |
| ATOM | 998 | HG1 | THR | A | 231 | 20.801 | 58.098 | −5.037 | 1.00 | 0.00 | H |
| ATOM | 999 | N | ALA | A | 232 | 26.140 | 57.513 | −4.773 | 1.00 | 0.00 | N |
| ATOM | 1000 | CA | ALA | A | 232 | 27.437 | 56.917 | −5.048 | 1.00 | 0.00 | C |
| ATOM | 1001 | C | ALA | A | 232 | 28.540 | 57.676 | −4.305 | 1.00 | 0.00 | C |
| ATOM | 1002 | O | ALA | A | 232 | 29.715 | 57.322 | −4.398 | 1.00 | 0.00 | O |
| ATOM | 1003 | CB | ALA | A | 232 | 27.693 | 56.951 | −6.558 | 1.00 | 0.00 | C |
| ATOM | 1004 | H | ALA | A | 232 | 26.066 | 58.510 | −4.920 | 1.00 | 0.00 | H |
| ATOM | 1005 | HA | ALA | A | 232 | 27.430 | 55.871 | −4.742 | 1.00 | 0.00 | H |
| ATOM | 1006 | 1HB | ALA | A | 232 | 28.664 | 56.505 | −6.772 | 1.00 | 0.00 | H |
| ATOM | 1007 | 2HB | ALA | A | 232 | 26.914 | 56.387 | −7.071 | 1.00 | 0.00 | H |
| ATOM | 1008 | 3HB | ALA | A | 232 | 27.682 | 57.983 | −6.906 | 1.00 | 0.00 | H |
| ATOM | 1009 | N | ALA | A | 233 | 28.185 | 58.748 | −3.597 | 1.00 | 0.00 | N |
| ATOM | 1010 | CA | ALA | A | 233 | 29.153 | 59.681 | −3.050 | 1.00 | 0.00 | C |
| ATOM | 1011 | C | ALA | A | 233 | 29.873 | 59.069 | −1.858 | 1.00 | 0.00 | C |
| ATOM | 1012 | O | ALA | A | 233 | 31.019 | 59.410 | −1.577 | 1.00 | 0.00 | O |
| ATOM | 1013 | CB | ALA | A | 233 | 28.451 | 60.972 | −2.623 | 1.00 | 0.00 | C |
| ATOM | 1014 | H | ALA | A | 233 | 27.208 | 58.938 | −3.424 | 1.00 | 0.00 | H |
| ATOM | 1015 | HA | ALA | A | 233 | 29.874 | 59.952 | −3.821 | 1.00 | 0.00 | H |
| ATOM | 1016 | 1HB | ALA | A | 233 | 29.184 | 61.667 | −2.213 | 1.00 | 0.00 | H |
| ATOM | 1017 | 2HB | ALA | A | 233 | 27.966 | 61.425 | −3.488 | 1.00 | 0.00 | H |
| ATOM | 1018 | 3HB | ALA | A | 233 | 27.701 | 60.746 | −1.865 | 1.00 | 0.00 | H |
| ATOM | 1019 | N | PHE | A | 234 | 29.174 | 58.196 | −1.140 | 1.00 | 0.00 | N |
| ATOM | 1020 | CA | PHE | A | 234 | 29.685 | 57.561 | 0.056 | 1.00 | 0.00 | C |
| ATOM | 1021 | C | PHE | A | 234 | 30.371 | 56.244 | −0.297 | 1.00 | 0.00 | C |
| ATOM | 1022 | O | PHE | A | 234 | 31.084 | 55.687 | 0.529 | 1.00 | 0.00 | O |
| ATOM | 1023 | CB | PHE | A | 234 | 28.536 | 57.341 | 1.043 | 1.00 | 0.00 | C |
| ATOM | 1024 | CG | PHE | A | 234 | 27.297 | 56.711 | 0.438 | 1.00 | 0.00 | C |
| ATOM | 1025 | CD1 | PHE | A | 234 | 27.232 | 55.323 | 0.213 | 1.00 | 0.00 | C |
| ATOM | 1026 | CD2 | PHE | A | 234 | 26.188 | 57.508 | 0.091 | 1.00 | 0.00 | C |
| ATOM | 1027 | CE1 | PHE | A | 234 | 26.077 | 54.743 | −0.336 | 1.00 | 0.00 | C |
| ATOM | 1028 | CE2 | PHE | A | 234 | 25.027 | 56.929 | −0.443 | 1.00 | 0.00 | C |
| ATOM | 1029 | CZ | PHE | A | 234 | 24.971 | 55.545 | −0.662 | 1.00 | 0.00 | C |
| ATOM | 1030 | H | PHE | A | 234 | 28.236 | 57.947 | −1.422 | 1.00 | 0.00 | H |
| ATOM | 1031 | HA | PHE | A | 234 | 30.371 | 58.239 | 0.564 | 1.00 | 0.00 | H |
| ATOM | 1032 | 1HB | PHE | A | 234 | 28.867 | 56.681 | 1.845 | 1.00 | 0.00 | H |
| ATOM | 1033 | 2HB | PHE | A | 234 | 28.230 | 58.299 | 1.465 | 1.00 | 0.00 | H |
| ATOM | 1034 | HD1 | PHE | A | 234 | 28.076 | 54.696 | 0.462 | 1.00 | 0.00 | H |
| ATOM | 1035 | HD2 | PHE | A | 234 | 26.226 | 58.578 | 0.235 | 1.00 | 0.00 | H |
| ATOM | 1036 | HE1 | PHE | A | 234 | 26.039 | 53.677 | −0.509 | 1.00 | 0.00 | H |
| ATOM | 1037 | HE2 | PHE | A | 234 | 24.177 | 57.549 | −0.685 | 1.00 | 0.00 | H |
| ATOM | 1038 | HZ | PHE | A | 234 | 24.082 | 55.095 | −1.079 | 1.00 | 0.00 | H |
| ATOM | 1039 | N | LEU | A | 235 | 30.157 | 55.721 | −1.504 | 1.00 | 0.00 | N |
| ATOM | 1040 | CA | LEU | A | 235 | 30.708 | 54.426 | −1.867 | 1.00 | 0.00 | C |
| ATOM | 1041 | C | LEU | A | 235 | 32.224 | 54.529 | −2.000 | 1.00 | 0.00 | C |
| ATOM | 1042 | O | LEU | A | 235 | 32.951 | 53.752 | −1.388 | 1.00 | 0.00 | O |
| ATOM | 1043 | CB | LEU | A | 235 | 30.069 | 53.888 | −3.154 | 1.00 | 0.00 | C |
| ATOM | 1044 | CG | LEU | A | 235 | 28.600 | 53.471 | −2.969 | 1.00 | 0.00 | C |
| ATOM | 1045 | CD1 | LEU | A | 235 | 28.038 | 53.022 | −4.320 | 1.00 | 0.00 | C |
| ATOM | 1046 | CD2 | LEU | A | 235 | 28.461 | 52.313 | −1.969 | 1.00 | 0.00 | C |
| ATOM | 1047 | H | LEU | A | 235 | 29.604 | 56.225 | −2.183 | 1.00 | 0.00 | H |
| ATOM | 1048 | HA | LEU | A | 235 | 30.405 | 53.682 | −1.130 | 1.00 | 0.00 | H |
| ATOM | 1049 | 1HB | LEU | A | 235 | 30.099 | 54.658 | −3.924 | 1.00 | 0.00 | H |
| ATOM | 1050 | 2HB | LEU | A | 235 | 30.620 | 53.012 | −3.495 | 1.00 | 0.00 | H |
| ATOM | 1051 | HG | LEU | A | 235 | 28.026 | 54.319 | −2.595 | 1.00 | 0.00 | H |
| ATOM | 1052 | 1HD1 | LEU | A | 235 | 26.997 | 52.724 | −4.200 | 1.00 | 0.00 | H |
| ATOM | 1053 | 2HD1 | LEU | A | 235 | 28.101 | 53.845 | −5.032 | 1.00 | 0.00 | H |
| ATOM | 1054 | 3HD1 | LEU | A | 235 | 28.617 | 52.176 | −4.692 | 1.00 | 0.00 | H |
| ATOM | 1055 | 1HD2 | LEU | A | 235 | 27.409 | 52.047 | −1.865 | 1.00 | 0.00 | H |
| ATOM | 1056 | 2HD2 | LEU | A | 235 | 29.019 | 51.450 | −2.332 | 1.00 | 0.00 | H |
| ATOM | 1057 | 3HD2 | LEU | A | 235 | 28.855 | 52.619 | −1.000 | 1.00 | 0.00 | H |
| ATOM | 1058 | N | LEU | A | 236 | 32.696 | 55.466 | −2.821 | 1.00 | 0.00 | N |
| ATOM | 1059 | CA | LEU | A | 236 | 34.120 | 55.663 | −3.048 | 1.00 | 0.00 | C |
| ATOM | 1060 | C | LEU | A | 236 | 34.903 | 55.781 | −1.728 | 1.00 | 0.00 | C |
| ATOM | 1061 | O | LEU | A | 236 | 35.898 | 55.076 | −1.556 | 1.00 | 0.00 | O |
| ATOM | 1062 | CB | LEU | A | 236 | 34.376 | 56.841 | −4.002 | 1.00 | 0.00 | C |
| ATOM | 1063 | CG | LEU | A | 236 | 33.635 | 56.740 | −5.348 | 1.00 | 0.00 | C |
| ATOM | 1064 | CD1 | LEU | A | 236 | 33.979 | 57.968 | −6.197 | 1.00 | 0.00 | C |
| ATOM | 1065 | CD2 | LEU | A | 236 | 34.012 | 55.479 | −6.132 | 1.00 | 0.00 | C |
| ATOM | 1066 | H | LEU | A | 236 | 32.058 | 56.073 | −3.315 | 1.00 | 0.00 | H |
| ATOM | 1067 | HA | LEU | A | 236 | 34.493 | 54.885 | −3.715 | 1.00 | 0.00 | H |
| ATOM | 1068 | 1HB | LEU | A | 236 | 34.052 | 57.769 | −3.529 | 1.00 | 0.00 | H |
| ATOM | 1069 | 2HB | LEU | A | 236 | 35.440 | 56.902 | −4.228 | 1.00 | 0.00 | H |
| ATOM | 1070 | HG | LEU | A | 236 | 32.560 | 56.695 | −5.169 | 1.00 | 0.00 | H |
| ATOM | 1071 | 1HD1 | LEU | A | 236 | 33.460 | 57.907 | −7.153 | 1.00 | 0.00 | H |
| ATOM | 1072 | 2HD1 | LEU | A | 236 | 33.669 | 58.872 | −5.672 | 1.00 | 0.00 | H |
| ATOM | 1073 | 3HD1 | LEU | A | 236 | 35.055 | 58.000 | −6.370 | 1.00 | 0.00 | H |
| ATOM | 1074 | 1HD2 | LEU | A | 236 | 33.461 | 55.456 | −7.072 | 1.00 | 0.00 | H |
| ATOM | 1075 | 2HD2 | LEU | A | 236 | 35.082 | 55.485 | −6.338 | 1.00 | 0.00 | H |
| ATOM | 1076 | 3HD2 | LEU | A | 236 | 33.760 | 54.596 | −5.544 | 1.00 | 0.00 | H |
| ATOM | 1077 | N | PRO | A | 237 | 34.501 | 56.633 | −0.771 | 1.00 | 0.00 | N |
| ATOM | 1078 | CA | PRO | A | 237 | 35.275 | 56.797 | 0.442 | 1.00 | 0.00 | C |
| ATOM | 1079 | C | PRO | A | 237 | 35.127 | 55.560 | 1.325 | 1.00 | 0.00 | C |
| ATOM | 1080 | O | PRO | A | 237 | 36.117 | 55.091 | 1.872 | 1.00 | 0.00 | O |
| ATOM | 1081 | CB | PRO | A | 237 | 34.760 | 58.064 | 1.126 | 1.00 | 0.00 | C |
| ATOM | 1082 | CG | PRO | A | 237 | 33.337 | 58.183 | 0.588 | 1.00 | 0.00 | C |
| ATOM | 1083 | CD | PRO | A | 237 | 33.495 | 57.676 | −0.844 | 1.00 | 0.00 | C |
| ATOM | 1084 | HA | PRO | A | 237 | 36.320 | 56.970 | 0.186 | 1.00 | 0.00 | H |
| ATOM | 1085 | 1HB | PRO | A | 237 | 34.792 | 57.933 | 2.207 | 1.00 | 0.00 | H |
| ATOM | 1086 | 2HB | PRO | A | 237 | 35.388 | 58.910 | 0.844 | 1.00 | 0.00 | H |
| ATOM | 1087 | 1HG | PRO | A | 237 | 32.668 | 57.566 | 1.188 | 1.00 | 0.00 | H |
| ATOM | 1088 | 2HG | PRO | A | 237 | 33.014 | 59.223 | 0.639 | 1.00 | 0.00 | H |
| ATOM | 1089 | 1HD | PRO | A | 237 | 33.838 | 58.465 | −1.513 | 1.00 | 0.00 | H |
| ATOM | 1090 | 2HD | PRO | A | 237 | 32.564 | 57.257 | −1.225 | 1.00 | 0.00 | H |
| ATOM | 1091 | N | ILE | A | 238 | 33.928 | 54.989 | 1.451 | 1.00 | 0.00 | N |
| ATOM | 1092 | CA | ILE | A | 238 | 33.752 | 53.784 | 2.251 | 1.00 | 0.00 | C |
| ATOM | 1093 | C | ILE | A | 238 | 34.655 | 52.676 | 1.715 | 1.00 | 0.00 | C |
| ATOM | 1094 | O | ILE | A | 238 | 35.204 | 51.894 | 2.488 | 1.00 | 0.00 | O |
| ATOM | 1095 | CB | ILE | A | 238 | 32.271 | 53.368 | 2.282 | 1.00 | 0.00 | C |
| ATOM | 1096 | CG1 | ILE | A | 238 | 31.533 | 54.338 | 3.220 | 1.00 | 0.00 | C |
| ATOM | 1097 | CG2 | ILE | A | 238 | 32.062 | 51.919 | 2.744 | 1.00 | 0.00 | C |
| ATOM | 1098 | CD1 | ILE | A | 238 | 30.014 | 54.203 | 3.132 | 1.00 | 0.00 | C |
| ATOM | 1099 | H | ILE | A | 238 | 33.126 | 55.391 | 0.987 | 1.00 | 0.00 | H |
| ATOM | 1100 | HA | ILE | A | 238 | 33.939 | 54.013 | 3.300 | 1.00 | 0.00 | H |
| ATOM | 1101 | HB | ILE | A | 238 | 31.866 | 53.390 | 1.271 | 1.00 | 0.00 | H |
| ATOM | 1102 | 1HG1 | ILE | A | 238 | 31.825 | 54.140 | 4.252 | 1.00 | 0.00 | H |
| ATOM | 1103 | 2HG1 | ILE | A | 238 | 31.792 | 55.364 | 2.961 | 1.00 | 0.00 | H |
| ATOM | 1104 | 1HG2 | ILE | A | 238 | 30.997 | 51.687 | 2.744 | 1.00 | 0.00 | H |
| ATOM | 1105 | 2HG2 | ILE | A | 238 | 32.581 | 51.243 | 2.064 | 1.00 | 0.00 | H |
| ATOM | 1106 | 3HG2 | ILE | A | 238 | 32.461 | 51.797 | 3.751 | 1.00 | 0.00 | H |
| ATOM | 1107 | 1HD1 | ILE | A | 238 | 29.545 | 54.911 | 3.815 | 1.00 | 0.00 | H |
| ATOM | 1108 | 2HD1 | ILE | A | 238 | 29.689 | 54.413 | 2.113 | 1.00 | 0.00 | H |
| ATOM | 1109 | 3HD1 | ILE | A | 238 | 29.722 | 53.189 | 3.404 | 1.00 | 0.00 | H |
| ATOM | 1110 | N | LEU | A | 239 | 34.794 | 52.590 | 0.396 | 1.00 | 0.00 | N |
| ATOM | 1111 | CA | LEU | A | 239 | 35.675 | 51.620 | −0.215 | 1.00 | 0.00 | C |
| ATOM | 1112 | C | LEU | A | 239 | 37.129 | 51.986 | 0.091 | 1.00 | 0.00 | C |
| ATOM | 1113 | O | LEU | A | 239 | 37.915 | 51.095 | 0.403 | 1.00 | 0.00 | O |
| ATOM | 1114 | CB | LEU | A | 239 | 35.365 | 51.493 | −1.710 | 1.00 | 0.00 | C |
| ATOM | 1115 | CG | LEU | A | 239 | 33.980 | 50.862 | −1.953 | 1.00 | 0.00 | C |
| ATOM | 1116 | CD1 | LEU | A | 239 | 33.526 | 51.171 | −3.380 | 1.00 | 0.00 | C |
| ATOM | 1117 | CD2 | LEU | A | 239 | 33.994 | 49.340 | −1.762 | 1.00 | 0.00 | C |
| ATOM | 1118 | H | LEU | A | 239 | 34.278 | 53.214 | −0.208 | 1.00 | 0.00 | H |
| ATOM | 1119 | HA | LEU | A | 239 | 35.388 | 50.617 | 0.103 | 1.00 | 0.00 | H |
| ATOM | 1120 | 1HB | LEU | A | 239 | 35.377 | 52.482 | −2.168 | 1.00 | 0.00 | H |
| ATOM | 1121 | 2HB | LEU | A | 239 | 36.118 | 50.864 | −2.185 | 1.00 | 0.00 | H |
| ATOM | 1122 | HG | LEU | A | 239 | 33.265 | 51.267 | −1.237 | 1.00 | 0.00 | H |
| ATOM | 1123 | 1HD1 | LEU | A | 239 | 32.547 | 50.726 | −3.555 | 1.00 | 0.00 | H |
| ATOM | 1124 | 2HD1 | LEU | A | 239 | 33.463 | 52.251 | −3.515 | 1.00 | 0.00 | H |
| ATOM | 1125 | 3HD1 | LEU | A | 239 | 34.245 | 50.758 | −4.088 | 1.00 | 0.00 | H |
| ATOM | 1126 | 1HD2 | LEU | A | 239 | 32.996 | 48.941 | −1.944 | 1.00 | 0.00 | H |
| ATOM | 1127 | 2HD2 | LEU | A | 239 | 34.698 | 48.891 | −2.464 | 1.00 | 0.00 | H |
| ATOM | 1128 | 3HD2 | LEU | A | 239 | 34.299 | 49.103 | −0.742 | 1.00 | 0.00 | H |
| ATOM | 1129 | N | SER | A | 240 | 37.490 | 53.274 | 0.067 | 1.00 | 0.00 | N |
| ATOM | 1130 | CA | SER | A | 240 | 38.789 | 53.711 | 0.567 | 1.00 | 0.00 | C |
| ATOM | 1131 | C | SER | A | 240 | 39.004 | 53.187 | 1.992 | 1.00 | 0.00 | C |
| ATOM | 1132 | O | SER | A | 240 | 40.069 | 52.659 | 2.297 | 1.00 | 0.00 | O |
| ATOM | 1133 | CB | SER | A | 240 | 38.956 | 55.242 | 0.509 | 1.00 | 0.00 | C |
| ATOM | 1134 | OG | SER | A | 240 | 40.271 | 55.647 | 0.866 | 1.00 | 0.00 | O |
| ATOM | 1135 | H | SER | A | 240 | 36.858 | 53.969 | −0.304 | 1.00 | 0.00 | H |
| ATOM | 1136 | HA | SER | A | 240 | 39.571 | 53.389 | −0.121 | 1.00 | 0.00 | H |
| ATOM | 1137 | 1HB | SER | A | 240 | 38.756 | 55.592 | −0.504 | 1.00 | 0.00 | H |
| ATOM | 1138 | 2HB | SER | A | 240 | 38.256 | 55.711 | 1.200 | 1.00 | 0.00 | H |
| ATOM | 1139 | HG | SER | A | 240 | 40.335 | 56.604 | 0.818 | 1.00 | 0.00 | H |
| ATOM | 1140 | N | GLN | A | 241 | 37.997 | 53.276 | 2.865 | 1.00 | 0.00 | N |
| ATOM | 1141 | CA | GLN | A | 241 | 38.109 | 52.795 | 4.240 | 1.00 | 0.00 | C |
| ATOM | 1142 | C | GLN | A | 241 | 38.088 | 51.262 | 4.322 | 1.00 | 0.00 | C |
| ATOM | 1143 | O | GLN | A | 241 | 38.033 | 50.698 | 5.415 | 1.00 | 0.00 | O |
| ATOM | 1144 | CB | GLN | A | 241 | 37.005 | 53.369 | 5.135 | 1.00 | 0.00 | C |
| ATOM | 1145 | CG | GLN | A | 241 | 36.801 | 54.888 | 5.108 | 1.00 | 0.00 | C |
| ATOM | 1146 | CD | GLN | A | 241 | 38.015 | 55.722 | 5.492 | 1.00 | 0.00 | C |
| ATOM | 1147 | NE2 | GLN | A | 241 | 37.778 | 56.760 | 6.278 | 1.00 | 0.00 | N |
| ATOM | 1148 | OE1 | GLN | A | 241 | 39.140 | 55.495 | 5.043 | 1.00 | 0.00 | O |
| ATOM | 1149 | H | GLN | A | 241 | 37.119 | 53.688 | 2.583 | 1.00 | 0.00 | H |
| ATOM | 1150 | HA | GLN | A | 241 | 39.016 | 53.194 | 4.693 | 1.00 | 0.00 | H |
| ATOM | 1151 | 1HB | GLN | A | 241 | 36.044 | 52.941 | 4.848 | 1.00 | 0.00 | H |
| ATOM | 1152 | 2HB | GLN | A | 241 | 37.215 | 53.122 | 6.176 | 1.00 | 0.00 | H |
| ATOM | 1153 | 1HG | GLN | A | 241 | 36.522 | 55.200 | 4.102 | 1.00 | 0.00 | H |
| ATOM | 1154 | 2HG | GLN | A | 241 | 36.010 | 55.162 | 5.806 | 1.00 | 0.00 | H |
| ATOM | 1155 | 1HE2 | GLN | A | 241 | 38.539 | 57.356 | 6.571 | 1.00 | 0.00 | H |
| ATOM | 1156 | 2HE2 | GLN | A | 241 | 36.836 | 56.956 | 6.585 | 1.00 | 0.00 | H |
| ATOM | 1157 | N | ILE | A | 242 | 38.115 | 50.566 | 3.186 | 1.00 | 0.00 | N |
| ATOM | 1158 | CA | ILE | A | 242 | 38.313 | 49.133 | 3.115 | 1.00 | 0.00 | C |
| ATOM | 1159 | C | ILE | A | 242 | 39.590 | 48.864 | 2.302 | 1.00 | 0.00 | C |
| ATOM | 1160 | O | ILE | A | 242 | 40.084 | 47.735 | 2.277 | 1.00 | 0.00 | O |
| ATOM | 1161 | CB | ILE | A | 242 | 37.045 | 48.444 | 2.563 | 1.00 | 0.00 | C |
| ATOM | 1162 | CG1 | ILE | A | 242 | 35.831 | 48.732 | 3.470 | 1.00 | 0.00 | C |
| ATOM | 1163 | CG2 | ILE | A | 242 | 37.255 | 46.924 | 2.491 | 1.00 | 0.00 | C |
| ATOM | 1164 | CD1 | ILE | A | 242 | 34.483 | 48.423 | 2.808 | 1.00 | 0.00 | C |
| ATOM | 1165 | H | ILE | A | 242 | 37.992 | 51.039 | 2.302 | 1.00 | 0.00 | H |
| ATOM | 1166 | HA | ILE | A | 242 | 38.313 | 48.715 | 4.121 | 1.00 | 0.00 | H |
| ATOM | 1167 | HB | ILE | A | 242 | 36.836 | 48.816 | 1.560 | 1.00 | 0.00 | H |
| ATOM | 1168 | 1HG1 | ILE | A | 242 | 35.897 | 48.123 | 4.372 | 1.00 | 0.00 | H |
| ATOM | 1169 | 2HG1 | ILE | A | 242 | 35.824 | 49.787 | 3.745 | 1.00 | 0.00 | H |
| ATOM | 1170 | 1HG2 | ILE | A | 242 | 36.355 | 46.450 | 2.100 | 1.00 | 0.00 | H |
| ATOM | 1171 | 2HG2 | ILE | A | 242 | 38.096 | 46.703 | 1.833 | 1.00 | 0.00 | H |
| ATOM | 1172 | 3HG2 | ILE | A | 242 | 37.464 | 46.538 | 3.489 | 1.00 | 0.00 | H |
| ATOM | 1173 | 1HD1 | ILE | A | 242 | 33.675 | 48.650 | 3.504 | 1.00 | 0.00 | H |
| ATOM | 1174 | 2HD1 | ILE | A | 242 | 34.370 | 49.032 | 1.911 | 1.00 | 0.00 | H |
| ATOM | 1175 | 3HD1 | ILE | A | 242 | 34.443 | 47.368 | 2.537 | 1.00 | 0.00 | H |
| ATOM | 1176 | N | TYR | A | 243 | 40.187 | 49.898 | 1.713 | 1.00 | 0.00 | N |
| ATOM | 1177 | CA | TYR | A | 243 | 41.500 | 49.888 | 1.076 | 1.00 | 0.00 | C |
| ATOM | 1178 | C | TYR | A | 243 | 42.446 | 50.665 | 1.998 | 1.00 | 0.00 | C |
| ATOM | 1179 | O | TYR | A | 243 | 43.404 | 51.297 | 1.567 | 1.00 | 0.00 | O |
| ATOM | 1180 | CB | TYR | A | 243 | 41.405 | 50.476 | −0.342 | 1.00 | 0.00 | C |
| ATOM | 1181 | CG | TYR | A | 243 | 40.296 | 49.900 | −1.215 | 1.00 | 0.00 | C |
| ATOM | 1182 | CD1 | TYR | A | 243 | 39.827 | 48.584 | −1.027 | 1.00 | 0.00 | C |
| ATOM | 1183 | CD2 | TYR | A | 243 | 39.693 | 50.684 | −2.218 | 1.00 | 0.00 | C |
| ATOM | 1184 | CE1 | TYR | A | 243 | 38.738 | 48.097 | −1.762 | 1.00 | 0.00 | C |
| ATOM | 1185 | CE2 | TYR | A | 243 | 38.632 | 50.178 | −2.991 | 1.00 | 0.00 | C |
| ATOM | 1186 | CZ | TYR | A | 243 | 38.136 | 48.881 | −2.757 | 1.00 | 0.00 | C |
| ATOM | 1187 | OH | TYR | A | 243 | 37.095 | 48.364 | −3.474 | 1.00 | 0.00 | O |
| ATOM | 1188 | H | TYR | A | 243 | 39.725 | 50.795 | 1.679 | 1.00 | 0.00 | H |
| ATOM | 1189 | HA | TYR | A | 243 | 41.809 | 48.859 | 0.895 | 1.00 | 0.00 | H |
| ATOM | 1190 | 1HB | TYR | A | 243 | 41.222 | 51.549 | −0.280 | 1.00 | 0.00 | H |
| ATOM | 1191 | 2HB | TYR | A | 243 | 42.340 | 50.298 | −0.874 | 1.00 | 0.00 | H |
| ATOM | 1192 | HD1 | TYR | A | 243 | 40.307 | 47.937 | −0.309 | 1.00 | 0.00 | H |
| ATOM | 1193 | HD2 | TYR | A | 243 | 40.045 | 51.689 | −2.402 | 1.00 | 0.00 | H |
| ATOM | 1194 | HE1 | TYR | A | 243 | 38.352 | 47.108 | −1.566 | 1.00 | 0.00 | H |
| ATOM | 1195 | HE2 | TYR | A | 243 | 38.193 | 50.785 | −3.769 | 1.00 | 0.00 | H |
| ATOM | 1196 | HH | TYR | A | 243 | 36.792 | 49.014 | −4.112 | 1.00 | 0.00 | H |
| ATOM | 1197 | N | SER | A | 244 | 42.129 | 50.577 | 3.288 | 1.00 | 0.00 | N |
| ATOM | 1198 | CA | SER | A | 244 | 42.651 | 51.258 | 4.453 | 1.00 | 0.00 | C |
| ATOM | 1199 | C | SER | A | 244 | 41.782 | 50.695 | 5.594 | 1.00 | 0.00 | C |
| ATOM | 1200 | O | SER | A | 244 | 41.188 | 49.622 | 5.406 | 1.00 | 0.00 | O |
| ATOM | 1201 | CB | SER | A | 244 | 42.512 | 52.779 | 4.288 | 1.00 | 0.00 | C |
| ATOM | 1202 | OG | SER | A | 244 | 43.178 | 53.491 | 5.313 | 1.00 | 0.00 | O |
| ATOM | 1203 | H | SER | A | 244 | 41.402 | 49.942 | 3.587 | 1.00 | 0.00 | H |
| ATOM | 1204 | HA | SER | A | 244 | 43.710 | 51.028 | 4.564 | 1.00 | 0.00 | H |
| ATOM | 1205 | 1HB | SER | A | 244 | 42.940 | 53.082 | 3.332 | 1.00 | 0.00 | H |
| ATOM | 1206 | 2HB | SER | A | 244 | 41.457 | 53.053 | 4.316 | 1.00 | 0.00 | H |
| ATOM | 1207 | HG | SER | A | 244 | 43.067 | 54.434 | 5.173 | 1.00 | 0.00 | H |
| ATOM | 1208 | N | ASP | A | 245 | 41.685 | 51.427 | 6.712 | 1.00 | 0.00 | N |
| ATOM | 1209 | CA | ASP | A | 245 | 40.956 | 51.110 | 7.948 | 1.00 | 0.00 | C |
| ATOM | 1210 | C | ASP | A | 245 | 40.531 | 49.642 | 8.056 | 1.00 | 0.00 | C |
| ATOM | 1211 | O | ASP | A | 245 | 41.306 | 48.823 | 8.551 | 1.00 | 0.00 | O |
| ATOM | 1212 | CB | ASP | A | 245 | 39.779 | 52.075 | 8.184 | 1.00 | 0.00 | C |
| ATOM | 1213 | CG | ASP | A | 245 | 38.905 | 51.658 | 9.368 | 1.00 | 0.00 | C |
| ATOM | 1214 | OD1 | ASP | A | 245 | 37.675 | 51.858 | 9.265 | 1.00 | 0.00 | O |
| ATOM | 1215 | OD2 | ASP | A | 245 | 39.448 | 51.130 | 10.358 | 1.00 | 0.00 | O1− |
| ATOM | 1216 | H | ASP | A | 245 | 42.152 | 52.320 | 6.773 | 1.00 | 0.00 | H |
| ATOM | 1217 | HA | ASP | A | 245 | 41.525 | 51.465 | 8.807 | 1.00 | 0.00 | H |
| ATOM | 1218 | 1HB | ASP | A | 245 | 40.163 | 53.075 | 8.387 | 1.00 | 0.00 | H |
| ATOM | 1219 | 2HB | ASP | A | 245 | 39.148 | 52.103 | 7.296 | 1.00 | 0.00 | H |
| ATOM | 1220 | N | GLY | A | 246 | 39.306 | 49.342 | 7.601 | 1.00 | 0.00 | N |
| ATOM | 1221 | CA | GLY | A | 246 | 38.538 | 48.134 | 7.842 | 1.00 | 0.00 | C |
| ATOM | 1222 | C | GLY | A | 246 | 39.412 | 46.949 | 8.261 | 1.00 | 0.00 | C |
| ATOM | 1223 | O | GLY | A | 246 | 39.985 | 46.301 | 7.384 | 1.00 | 0.00 | O |
| ATOM | 1224 | H | GLY | A | 246 | 38.809 | 49.998 | 7.016 | 1.00 | 0.00 | H |
| ATOM | 1225 | 1HA | GLY | A | 246 | 37.817 | 48.313 | 8.639 | 1.00 | 0.00 | H |
| ATOM | 1226 | 2HA | GLY | A | 246 | 38.010 | 47.851 | 6.931 | 1.00 | 0.00 | H |
| ATOM | 1227 | N | PRO | A | 247 | 39.529 | 46.638 | 9.561 | 1.00 | 0.00 | N |
| ATOM | 1228 | CA | PRO | A | 247 | 40.711 | 45.918 | 10.008 | 1.00 | 0.00 | C |
| ATOM | 1229 | C | PRO | A | 247 | 40.835 | 44.482 | 9.482 | 1.00 | 0.00 | C |
| ATOM | 1230 | O | PRO | A | 247 | 39.901 | 43.901 | 8.924 | 1.00 | 0.00 | O |
| ATOM | 1231 | CB | PRO | A | 247 | 40.660 | 45.971 | 11.533 | 1.00 | 0.00 | C |
| ATOM | 1232 | CG | PRO | A | 247 | 40.095 | 47.370 | 11.770 | 1.00 | 0.00 | C |
| ATOM | 1233 | CD | PRO | A | 247 | 39.100 | 47.546 | 10.622 | 1.00 | 0.00 | C |
| ATOM | 1234 | HA | PRO | A | 247 | 41.604 | 46.484 | 9.742 | 1.00 | 0.00 | H |
| ATOM | 1235 | 1HB | PRO | A | 247 | 40.012 | 45.177 | 11.904 | 1.00 | 0.00 | H |
| ATOM | 1236 | 2HB | PRO | A | 247 | 41.664 | 45.838 | 11.936 | 1.00 | 0.00 | H |
| ATOM | 1237 | 1HG | PRO | A | 247 | 39.614 | 47.407 | 12.747 | 1.00 | 0.00 | H |
| ATOM | 1238 | 2HG | PRO | A | 247 | 40.904 | 48.099 | 11.736 | 1.00 | 0.00 | H |
| ATOM | 1239 | 1HD | PRO | A | 247 | 39.112 | 48.571 | 10.250 | 1.00 | 0.00 | H |
| ATOM | 1240 | 2HD | PRO | A | 247 | 38.090 | 47.286 | 10.938 | 1.00 | 0.00 | H |
| ATOM | 1241 | N | GLY | A | 248 | 42.024 | 43.910 | 9.657 | 1.00 | 0.00 | N |
| ATOM | 1242 | CA | GLY | A | 248 | 42.370 | 42.615 | 9.107 | 1.00 | 0.00 | C |
| ATOM | 1243 | C | GLY | A | 248 | 41.581 | 41.494 | 9.784 | 1.00 | 0.00 | C |
| ATOM | 1244 | O | GLY | A | 248 | 41.257 | 41.580 | 10.969 | 1.00 | 0.00 | O |
| ATOM | 1245 | H | GLY | A | 248 | 42.737 | 44.382 | 10.194 | 1.00 | 0.00 | H |
| ATOM | 1246 | 1HA | GLY | A | 248 | 42.145 | 42.601 | 8.041 | 1.00 | 0.00 | H |
| ATOM | 1247 | 2HA | GLY | A | 248 | 43.433 | 42.428 | 9.257 | 1.00 | 0.00 | H |
| ATOM | 1248 | N | GLU | A | 249 | 41.296 | 40.431 | 9.027 | 1.00 | 0.00 | N |
| ATOM | 1249 | CA | GLU | A | 249 | 40.608 | 39.234 | 9.483 | 1.00 | 0.00 | C |
| ATOM | 1250 | C | GLU | A | 249 | 41.125 | 38.752 | 10.845 | 1.00 | 0.00 | C |
| ATOM | 1251 | O | GLU | A | 249 | 40.334 | 38.388 | 11.711 | 1.00 | 0.00 | O |
| ATOM | 1252 | CB | GLU | A | 249 | 40.771 | 38.144 | 8.418 | 1.00 | 0.00 | C |
| ATOM | 1253 | CG | GLU | A | 249 | 39.870 | 38.408 | 7.198 | 1.00 | 0.00 | C |
| ATOM | 1254 | CD | GLU | A | 249 | 38.482 | 37.828 | 7.409 | 1.00 | 0.00 | C |
| ATOM | 1255 | OE1 | GLU | A | 249 | 37.474 | 38.559 | 7.315 | 1.00 | 0.00 | O |
| ATOM | 1256 | OE2 | GLU | A | 249 | 38.397 | 36.612 | 7.677 | 1.00 | 0.00 | O1− |
| ATOM | 1257 | H | GLU | A | 249 | 41.564 | 40.417 | 8.053 | 1.00 | 0.00 | H |
| ATOM | 1258 | HA | GLU | A | 249 | 39.536 | 39.427 | 9.530 | 1.00 | 0.00 | H |
| ATOM | 1259 | 1HB | GLU | A | 249 | 41.807 | 38.117 | 8.081 | 1.00 | 0.00 | H |
| ATOM | 1260 | 2HB | GLU | A | 249 | 40.501 | 37.177 | 8.843 | 1.00 | 0.00 | H |
| ATOM | 1261 | 1HG | GLU | A | 249 | 39.779 | 39.482 | 7.039 | 1.00 | 0.00 | H |
| ATOM | 1262 | 2HG | GLU | A | 249 | 40.310 | 37.946 | 6.314 | 1.00 | 0.00 | H |
| ATOM | 1263 | N | ALA | A | 250 | 42.442 | 38.749 | 11.053 | 1.00 | 0.00 | N |
| ATOM | 1264 | CA | ALA | A | 250 | 43.035 | 38.279 | 12.298 | 1.00 | 0.00 | C |
| ATOM | 1265 | C | ALA | A | 250 | 42.938 | 39.368 | 13.373 | 1.00 | 0.00 | C |
| ATOM | 1266 | O | ALA | A | 250 | 43.952 | 39.820 | 13.892 | 1.00 | 0.00 | O |
| ATOM | 1267 | CB | ALA | A | 250 | 44.485 | 37.858 | 12.040 | 1.00 | 0.00 | C |
| ATOM | 1268 | H | ALA | A | 250 | 43.068 | 39.081 | 10.334 | 1.00 | 0.00 | H |
| ATOM | 1269 | HA | ALA | A | 250 | 42.542 | 37.358 | 12.611 | 1.00 | 0.00 | H |
| ATOM | 1270 | 1HB | ALA | A | 250 | 44.933 | 37.506 | 12.970 | 1.00 | 0.00 | H |
| ATOM | 1271 | 2HB | ALA | A | 250 | 44.506 | 37.057 | 11.301 | 1.00 | 0.00 | H |
| ATOM | 1272 | 3HB | ALA | A | 250 | 45.051 | 38.712 | 11.666 | 1.00 | 0.00 | H |
| ATOM | 1273 | N | LEU | A | 251 | 41.711 | 39.766 | 13.711 | 1.00 | 0.00 | N |
| ATOM | 1274 | CA | LEU | A | 251 | 41.340 | 40.820 | 14.647 | 1.00 | 0.00 | C |
| ATOM | 1275 | C | LEU | A | 251 | 39.860 | 41.093 | 14.394 | 1.00 | 0.00 | C |
| ATOM | 1276 | O | LEU | A | 251 | 39.013 | 40.932 | 15.273 | 1.00 | 0.00 | O |
| ATOM | 1277 | CB | LEU | A | 251 | 42.185 | 42.097 | 14.465 | 1.00 | 0.00 | C |
| ATOM | 1278 | CG | LEU | A | 251 | 41.646 | 43.323 | 15.224 | 1.00 | 0.00 | C |
| ATOM | 1279 | CD1 | LEU | A | 251 | 41.547 | 43.066 | 16.733 | 1.00 | 0.00 | C |
| ATOM | 1280 | CD2 | LEU | A | 251 | 42.580 | 44.513 | 14.980 | 1.00 | 0.00 | C |
| ATOM | 1281 | H | LEU | A | 251 | 40.904 | 39.319 | 13.299 | 1.00 | 0.00 | H |
| ATOM | 1282 | HA | LEU | A | 251 | 41.539 | 40.487 | 15.666 | 1.00 | 0.00 | H |
| ATOM | 1283 | 1HB | LEU | A | 251 | 43.198 | 41.918 | 14.826 | 1.00 | 0.00 | H |
| ATOM | 1284 | 2HB | LEU | A | 251 | 42.218 | 42.364 | 13.409 | 1.00 | 0.00 | H |
| ATOM | 1285 | HG | LEU | A | 251 | 40.657 | 43.579 | 14.845 | 1.00 | 0.00 | H |
| ATOM | 1286 | 1HD1 | LEU | A | 251 | 41.162 | 43.957 | 17.229 | 1.00 | 0.00 | H |
| ATOM | 1287 | 2HD1 | LEU | A | 251 | 40.873 | 42.229 | 16.916 | 1.00 | 0.00 | H |
| ATOM | 1288 | 3HD1 | LEU | A | 251 | 42.535 | 42.829 | 17.128 | 1.00 | 0.00 | H |
| ATOM | 1289 | 1HD2 | LEU | A | 251 | 42.205 | 45.386 | 15.514 | 1.00 | 0.00 | H |
| ATOM | 1290 | 2HD2 | LEU | A | 251 | 43.581 | 44.270 | 15.339 | 1.00 | 0.00 | H |
| ATOM | 1291 | 3HD2 | LEU | A | 251 | 42.621 | 44.731 | 13.912 | 1.00 | 0.00 | H |
| ATOM | 1292 | N | ARG | A | 252 | 39.536 | 41.486 | 13.163 | 1.00 | 0.00 | N |
| ATOM | 1293 | CA | ARG | A | 252 | 38.164 | 41.678 | 12.738 | 1.00 | 0.00 | C |
| ATOM | 1294 | C | ARG | A | 252 | 37.433 | 40.346 | 12.894 | 1.00 | 0.00 | C |
| ATOM | 1295 | O | ARG | A | 252 | 36.567 | 40.203 | 13.760 | 1.00 | 0.00 | O |
| ATOM | 1296 | CB | ARG | A | 252 | 38.130 | 42.231 | 11.300 | 1.00 | 0.00 | C |
| ATOM | 1297 | CG | ARG | A | 252 | 36.728 | 42.437 | 10.704 | 1.00 | 0.00 | C |
| ATOM | 1298 | CD | ARG | A | 252 | 35.806 | 43.223 | 11.648 | 1.00 | 0.00 | C |
| ATOM | 1299 | NE | ARG | A | 252 | 34.663 | 43.874 | 10.982 | 1.00 | 0.00 | N |
| ATOM | 1300 | CZ | ARG | A | 252 | 33.713 | 43.337 | 10.203 | 1.00 | 0.00 | C |
| ATOM | 1301 | NH1 | ARG | A | 252 | 33.751 | 42.082 | 9.779 | 1.00 | 0.00 | N1+ |
| ATOM | 1302 | NH2 | ARG | A | 252 | 32.695 | 44.110 | 9.855 | 1.00 | 0.00 | N |
| ATOM | 1303 | H | ARG | A | 252 | 40.260 | 41.663 | 12.482 | 1.00 | 0.00 | H |
| ATOM | 1304 | HA | ARG | A | 252 | 37.717 | 42.493 | 13.308 | 1.00 | 0.00 | H |
| ATOM | 1305 | 1HB | ARG | A | 252 | 38.621 | 43.204 | 11.273 | 1.00 | 0.00 | H |
| ATOM | 1306 | 2HB | ARG | A | 252 | 38.650 | 41.544 | 10.633 | 1.00 | 0.00 | H |
| ATOM | 1307 | 1HG | ARG | A | 252 | 36.808 | 42.992 | 9.769 | 1.00 | 0.00 | H |
| ATOM | 1308 | 2HG | ARG | A | 252 | 36.268 | 41.467 | 10.511 | 1.00 | 0.00 | H |
| ATOM | 1309 | 1HD | ARG | A | 252 | 35.394 | 42.549 | 12.399 | 1.00 | 0.00 | H |
| ATOM | 1310 | 2HD | ARG | A | 252 | 36.376 | 44.010 | 12.141 | 1.00 | 0.00 | H |
| ATOM | 1311 | HE | ARG | A | 252 | 34.652 | 44.864 | 11.183 | 1.00 | 0.00 | H |
| ATOM | 1312 | 1HH1 | ARG | A | 252 | 33.010 | 41.724 | 9.193 | 1.00 | 0.00 | H |
| ATOM | 1313 | 2HH1 | ARG | A | 252 | 34.520 | 41.483 | 10.043 | 1.00 | 0.00 | H |
| ATOM | 1314 | 1HH2 | ARG | A | 252 | 31.960 | 43.742 | 9.268 | 1.00 | 0.00 | H |
| ATOM | 1315 | 2HH2 | ARG | A | 252 | 32.655 | 45.066 | 10.175 | 1.00 | 0.00 | H |
| ATOM | 1316 | N | ALA | A | 253 | 37.834 | 39.370 | 12.086 | 1.00 | 0.00 | N |
| ATOM | 1317 | CA | ALA | A | 253 | 37.093 | 38.151 | 11.840 | 1.00 | 0.00 | C |
| ATOM | 1318 | C | ALA | A | 253 | 37.464 | 37.033 | 12.804 | 1.00 | 0.00 | C |
| ATOM | 1319 | O | ALA | A | 253 | 36.935 | 35.926 | 12.718 | 1.00 | 0.00 | O |
| ATOM | 1320 | CB | ALA | A | 253 | 37.275 | 37.724 | 10.394 | 1.00 | 0.00 | C |
| ATOM | 1321 | H | ALA | A | 253 | 38.712 | 39.452 | 11.593 | 1.00 | 0.00 | H |
| ATOM | 1322 | HA | ALA | A | 253 | 36.024 | 38.363 | 11.863 | 1.00 | 0.00 | H |
| ATOM | 1323 | 1HB | ALA | A | 253 | 36.716 | 36.806 | 10.213 | 1.00 | 0.00 | H |
| ATOM | 1324 | 2HB | ALA | A | 253 | 36.907 | 38.509 | 9.734 | 1.00 | 0.00 | H |
| ATOM | 1325 | 3HB | ALA | A | 253 | 38.333 | 37.549 | 10.197 | 1.00 | 0.00 | H |
| ATOM | 1326 | N | MET | A | 254 | 38.308 | 37.342 | 13.785 | 1.00 | 0.00 | N |
| ATOM | 1327 | CA | MET | A | 254 | 38.657 | 36.439 | 14.874 | 1.00 | 0.00 | C |
| ATOM | 1328 | C | MET | A | 254 | 37.545 | 36.461 | 15.934 | 1.00 | 0.00 | C |
| ATOM | 1329 | O | MET | A | 254 | 37.755 | 36.101 | 17.086 | 1.00 | 0.00 | O |
| ATOM | 1330 | CB | MET | A | 254 | 40.036 | 36.850 | 15.416 | 1.00 | 0.00 | C |
| ATOM | 1331 | CG | MET | A | 254 | 40.671 | 35.803 | 16.341 | 1.00 | 0.00 | C |
| ATOM | 1332 | SD | MET | A | 254 | 42.466 | 35.962 | 16.549 | 1.00 | 0.00 | S |
| ATOM | 1333 | CE | MET | A | 254 | 42.576 | 37.617 | 17.275 | 1.00 | 0.00 | C |
| ATOM | 1334 | H | MET | A | 254 | 38.750 | 38.249 | 13.805 | 1.00 | 0.00 | H |
| ATOM | 1335 | HA | MET | A | 254 | 38.846 | 35.443 | 14.474 | 1.00 | 0.00 | H |
| ATOM | 1336 | 1HB | MET | A | 254 | 40.723 | 37.006 | 14.584 | 1.00 | 0.00 | H |
| ATOM | 1337 | 2HB | MET | A | 254 | 39.942 | 37.773 | 15.988 | 1.00 | 0.00 | H |
| ATOM | 1338 | 1HG | MET | A | 254 | 40.231 | 35.882 | 17.336 | 1.00 | 0.00 | H |
| ATOM | 1339 | 2HG | MET | A | 254 | 40.487 | 34.805 | 15.942 | 1.00 | 0.00 | H |
| ATOM | 1340 | 1HE | MET | A | 254 | 43.621 | 37.864 | 17.463 | 1.00 | 0.00 | H |
| ATOM | 1341 | 2HE | MET | A | 254 | 42.149 | 38.346 | 16.586 | 1.00 | 0.00 | H |
| ATOM | 1342 | 3HE | MET | A | 254 | 42.024 | 37.640 | 18.215 | 1.00 | 0.00 | H |
| ATOM | 1343 | N | LYS | A | 255 | 36.355 | 36.928 | 15.553 | 1.00 | 0.00 | N |
| ATOM | 1344 | CA | LYS | A | 255 | 35.141 | 36.968 | 16.344 | 1.00 | 0.00 | C |
| ATOM | 1345 | C | LYS | A | 255 | 33.964 | 36.658 | 15.404 | 1.00 | 0.00 | C |
| ATOM | 1346 | O | LYS | A | 255 | 32.823 | 37.036 | 15.679 | 1.00 | 0.00 | O |
| ATOM | 1347 | CB | LYS | A | 255 | 34.994 | 38.381 | 16.929 | 1.00 | 0.00 | C |
| ATOM | 1348 | CG | LYS | A | 255 | 36.217 | 38.839 | 17.745 | 1.00 | 0.00 | C |
| ATOM | 1349 | CD | LYS | A | 255 | 36.040 | 40.245 | 18.342 | 1.00 | 0.00 | C |
| ATOM | 1350 | CE | LYS | A | 255 | 36.542 | 41.380 | 17.431 | 1.00 | 0.00 | C |
| ATOM | 1351 | NZ | LYS | A | 255 | 35.939 | 41.383 | 16.080 | 1.00 | 0.00 | N1+ |
| ATOM | 1352 | H | LYS | A | 255 | 36.234 | 37.305 | 14.624 | 1.00 | 0.00 | H |
| ATOM | 1353 | HA | LYS | A | 255 | 35.192 | 36.212 | 17.128 | 1.00 | 0.00 | H |
| ATOM | 1354 | 1HB | LYS | A | 255 | 34.856 | 39.098 | 16.120 | 1.00 | 0.00 | H |
| ATOM | 1355 | 2HB | LYS | A | 255 | 34.130 | 38.412 | 17.593 | 1.00 | 0.00 | H |
| ATOM | 1356 | 1HG | LYS | A | 255 | 36.384 | 38.146 | 18.570 | 1.00 | 0.00 | H |
| ATOM | 1357 | 2HG | LYS | A | 255 | 37.097 | 38.858 | 17.102 | 1.00 | 0.00 | H |
| ATOM | 1358 | 1HD | LYS | A | 255 | 34.982 | 40.429 | 18.532 | 1.00 | 0.00 | H |
| ATOM | 1359 | 2HD | LYS | A | 255 | 36.594 | 40.316 | 19.278 | 1.00 | 0.00 | H |
| ATOM | 1360 | 1HE | LYS | A | 255 | 36.309 | 42.343 | 17.887 | 1.00 | 0.00 | H |
| ATOM | 1361 | 2HE | LYS | A | 255 | 37.620 | 41.292 | 17.299 | 1.00 | 0.00 | H |
| ATOM | 1362 | 1HZ | LYS | A | 255 | 36.315 | 42.152 | 15.545 | 1.00 | 0.00 | H |
| ATOM | 1363 | 2HZ | LYS | A | 255 | 36.152 | 40.513 | 15.614 | 1.00 | 0.00 | H |
| ATOM | 1364 | 3HZ | LYS | A | 255 | 34.937 | 41.487 | 16.159 | 1.00 | 0.00 | H |
| ATOM | 1365 | N | GLU | A | 256 | 34.261 | 36.044 | 14.253 | 1.00 | 0.00 | N |
| ATOM | 1366 | CA | GLU | A | 256 | 33.325 | 35.873 | 13.152 | 1.00 | 0.00 | C |
| ATOM | 1367 | C | GLU | A | 256 | 33.543 | 34.516 | 12.478 | 1.00 | 0.00 | C |
| ATOM | 1368 | O | GLU | A | 256 | 32.590 | 33.821 | 12.128 | 1.00 | 0.00 | O |
| ATOM | 1369 | CB | GLU | A | 256 | 33.535 | 36.973 | 12.101 | 1.00 | 0.00 | C |
| ATOM | 1370 | CG | GLU | A | 256 | 33.583 | 38.410 | 12.642 | 1.00 | 0.00 | C |
| ATOM | 1371 | CD | GLU | A | 256 | 33.830 | 39.416 | 11.529 | 1.00 | 0.00 | C |
| ATOM | 1372 | OE1 | GLU | A | 256 | 33.712 | 39.055 | 10.338 | 1.00 | 0.00 | O |
| ATOM | 1373 | OE2 | GLU | A | 256 | 34.210 | 40.555 | 11.859 | 1.00 | 0.00 | O1− |
| ATOM | 1374 | H | GLU | A | 256 | 35.186 | 35.663 | 14.108 | 1.00 | 0.00 | H |
| ATOM | 1375 | HA | GLU | A | 256 | 32.304 | 35.913 | 13.533 | 1.00 | 0.00 | H |
| ATOM | 1376 | 1HB | GLU | A | 256 | 34.482 | 36.808 | 11.587 | 1.00 | 0.00 | H |
| ATOM | 1377 | 2HB | GLU | A | 256 | 32.721 | 36.945 | 11.377 | 1.00 | 0.00 | H |
| ATOM | 1378 | 1HG | GLU | A | 256 | 32.633 | 38.651 | 13.120 | 1.00 | 0.00 | H |
| ATOM | 1379 | 2HG | GLU | A | 256 | 34.389 | 38.498 | 13.371 | 1.00 | 0.00 | H |
| ATOM | 1380 | N | ASN | A | 257 | 34.807 | 34.167 | 12.242 | 1.00 | 0.00 | N |
| ATOM | 1381 | CA | ASN | A | 257 | 35.170 | 32.955 | 11.524 | 1.00 | 0.00 | C |
| ATOM | 1382 | C | ASN | A | 257 | 35.106 | 31.772 | 12.482 | 1.00 | 0.00 | C |
| ATOM | 1383 | O | ASN | A | 257 | 35.577 | 31.866 | 13.614 | 1.00 | 0.00 | O |
| ATOM | 1384 | CB | ASN | A | 257 | 36.591 | 33.059 | 10.949 | 1.00 | 0.00 | C |
| ATOM | 1385 | CG | ASN | A | 257 | 36.670 | 33.920 | 9.693 | 1.00 | 0.00 | C |
| ATOM | 1386 | ND2 | ASN | A | 257 | 37.779 | 34.628 | 9.505 | 1.00 | 0.00 | N |
| ATOM | 1387 | OD1 | ASN | A | 257 | 35.740 | 33.948 | 8.886 | 1.00 | 0.00 | O |
| ATOM | 1388 | H | ASN | A | 257 | 35.562 | 34.754 | 12.566 | 1.00 | 0.00 | H |
| ATOM | 1389 | HA | ASN | A | 257 | 34.475 | 32.800 | 10.698 | 1.00 | 0.00 | H |
| ATOM | 1390 | 1HB | ASN | A | 257 | 37.253 | 33.502 | 11.694 | 1.00 | 0.00 | H |
| ATOM | 1391 | 2HB | ASN | A | 257 | 36.952 | 32.064 | 10.689 | 1.00 | 0.00 | H |
| ATOM | 1392 | 1HD2 | ASN | A | 257 | 37.872 | 35.211 | 8.686 | 1.00 | 0.00 | H |
| ATOM | 1393 | 2HD2 | ASN | A | 257 | 38.528 | 34.583 | 10.181 | 1.00 | 0.00 | H |
| ATOM | 1394 | N | GLY | A | 258 | 34.585 | 30.635 | 12.029 | 1.00 | 0.00 | N |
| ATOM | 1395 | CA | GLY | A | 258 | 34.511 | 29.443 | 12.846 | 1.00 | 0.00 | C |
| ATOM | 1396 | C | GLY | A | 258 | 34.104 | 28.232 | 12.007 | 1.00 | 0.00 | C |
| ATOM | 1397 | O | GLY | A | 258 | 34.342 | 28.189 | 10.802 | 1.00 | 0.00 | O |
| ATOM | 1398 | H | GLY | A | 258 | 34.225 | 30.585 | 11.086 | 1.00 | 0.00 | H |
| ATOM | 1399 | 1HA | GLY | A | 258 | 35.485 | 29.248 | 13.294 | 1.00 | 0.00 | H |
| ATOM | 1400 | 2HA | GLY | A | 258 | 33.772 | 29.588 | 13.634 | 1.00 | 0.00 | H |
| ATOM | 1401 | N | ARG | A | 259 | 33.484 | 27.236 | 12.648 | 1.00 | 0.00 | N |
| ATOM | 1402 | CA | ARG | A | 259 | 33.355 | 25.871 | 12.142 | 1.00 | 0.00 | C |
| ATOM | 1403 | C | ARG | A | 259 | 32.606 | 25.760 | 10.805 | 1.00 | 0.00 | C |
| ATOM | 1404 | O | ARG | A | 259 | 32.595 | 24.688 | 10.208 | 1.00 | 0.00 | O |
| ATOM | 1405 | CB | ARG | A | 259 | 32.692 | 25.013 | 13.241 | 1.00 | 0.00 | C |
| ATOM | 1406 | CG | ARG | A | 259 | 32.557 | 23.520 | 12.895 | 1.00 | 0.00 | C |
| ATOM | 1407 | CD | ARG | A | 259 | 32.041 | 22.693 | 14.083 | 1.00 | 0.00 | C |
| ATOM | 1408 | NE | ARG | A | 259 | 33.046 | 22.541 | 15.154 | 1.00 | 0.00 | N |
| ATOM | 1409 | CZ | ARG | A | 259 | 34.012 | 21.609 | 15.189 | 1.00 | 0.00 | C |
| ATOM | 1410 | NH1 | ARG | A | 259 | 34.204 | 20.817 | 14.131 | 1.00 | 0.00 | N1+ |
| ATOM | 1411 | NH2 | ARG | A | 259 | 34.775 | 21.484 | 16.276 | 1.00 | 0.00 | N |
| ATOM | 1412 | H | ARG | A | 259 | 33.063 | 27.401 | 13.551 | 1.00 | 0.00 | H |
| ATOM | 1413 | HA | ARG | A | 259 | 34.342 | 25.412 | 12.074 | 1.00 | 0.00 | H |
| ATOM | 1414 | 1HB | ARG | A | 259 | 33.283 | 25.073 | 14.155 | 1.00 | 0.00 | H |
| ATOM | 1415 | 2HB | ARG | A | 259 | 31.686 | 25.384 | 13.435 | 1.00 | 0.00 | H |
| ATOM | 1416 | 1HG | ARG | A | 259 | 31.855 | 23.400 | 12.070 | 1.00 | 0.00 | H |
| ATOM | 1417 | 2HG | ARG | A | 259 | 33.530 | 23.124 | 12.604 | 1.00 | 0.00 | H |
| ATOM | 1418 | 1HD | ARG | A | 259 | 31.168 | 23.181 | 14.514 | 1.00 | 0.00 | H |
| ATOM | 1419 | 2HD | ARG | A | 259 | 31.767 | 21.695 | 13.740 | 1.00 | 0.00 | H |
| ATOM | 1420 | HE | ARG | A | 259 | 32.966 | 23.213 | 15.903 | 1.00 | 0.00 | H |
| ATOM | 1421 | 1HH1 | ARG | A | 259 | 34.929 | 20.114 | 14.152 | 1.00 | 0.00 | H |
| ATOM | 1422 | 2HH1 | ARG | A | 259 | 33.624 | 20.921 | 13.311 | 1.00 | 0.00 | H |
| ATOM | 1423 | 1HH2 | ARG | A | 259 | 35.502 | 20.784 | 16.306 | 1.00 | 0.00 | H |
| ATOM | 1424 | 2HH2 | ARG | A | 259 | 34.624 | 22.090 | 17.071 | 1.00 | 0.00 | H |
| ATOM | 1425 | N | TYR | A | 260 | 31.950 | 26.824 | 10.344 | 1.00 | 0.00 | N |
| ATOM | 1426 | CA | TYR | A | 260 | 31.140 | 26.805 | 9.134 | 1.00 | 0.00 | C |
| ATOM | 1427 | C | TYR | A | 260 | 31.498 | 28.025 | 8.282 | 1.00 | 0.00 | C |
| ATOM | 1428 | O | TYR | A | 260 | 30.663 | 28.577 | 7.560 | 1.00 | 0.00 | O |
| ATOM | 1429 | CB | TYR | A | 260 | 29.655 | 26.799 | 9.526 | 1.00 | 0.00 | C |
| ATOM | 1430 | CG | TYR | A | 260 | 29.302 | 25.790 | 10.604 | 1.00 | 0.00 | C |
| ATOM | 1431 | CD1 | TYR | A | 260 | 29.391 | 24.410 | 10.348 | 1.00 | 0.00 | C |
| ATOM | 1432 | CD2 | TYR | A | 260 | 28.906 | 26.215 | 11.888 | 1.00 | 0.00 | C |
| ATOM | 1433 | CE1 | TYR | A | 260 | 29.113 | 23.473 | 11.358 | 1.00 | 0.00 | C |
| ATOM | 1434 | CE2 | TYR | A | 260 | 28.618 | 25.279 | 12.896 | 1.00 | 0.00 | C |
| ATOM | 1435 | CZ | TYR | A | 260 | 28.732 | 23.900 | 12.643 | 1.00 | 0.00 | C |
| ATOM | 1436 | OH | TYR | A | 260 | 28.473 | 22.980 | 13.619 | 1.00 | 0.00 | O |
| ATOM | 1437 | H | TYR | A | 260 | 32.000 | 27.701 | 10.841 | 1.00 | 0.00 | H |
| ATOM | 1438 | HA | TYR | A | 260 | 31.312 | 25.874 | 8.595 | 1.00 | 0.00 | H |
| ATOM | 1439 | 1HB | TYR | A | 260 | 29.374 | 27.782 | 9.903 | 1.00 | 0.00 | H |
| ATOM | 1440 | 2HB | TYR | A | 260 | 29.049 | 26.558 | 8.652 | 1.00 | 0.00 | H |
| ATOM | 1441 | HD1 | TYR | A | 260 | 29.675 | 24.060 | 9.366 | 1.00 | 0.00 | H |
| ATOM | 1442 | HD2 | TYR | A | 260 | 28.820 | 27.269 | 12.105 | 1.00 | 0.00 | H |
| ATOM | 1443 | HE1 | TYR | A | 260 | 29.190 | 22.416 | 11.151 | 1.00 | 0.00 | H |
| ATOM | 1444 | HE2 | TYR | A | 260 | 28.306 | 25.617 | 13.873 | 1.00 | 0.00 | H |
| ATOM | 1445 | HH | TYR | A | 260 | 28.228 | 23.434 | 14.429 | 1.00 | 0.00 | H |
| ATOM | 1446 | N | GLY | A | 261 | 32.734 | 28.501 | 8.417 | 1.00 | 0.00 | N |
| ATOM | 1447 | CA | GLY | A | 261 | 33.154 | 29.737 | 7.794 | 1.00 | 0.00 | C |
| ATOM | 1448 | C | GLY | A | 261 | 32.597 | 30.873 | 8.642 | 1.00 | 0.00 | C |
| ATOM | 1449 | O | GLY | A | 261 | 33.032 | 31.050 | 9.774 | 1.00 | 0.00 | O |
| ATOM | 1450 | H | GLY | A | 261 | 33.412 | 27.996 | 8.968 | 1.00 | 0.00 | H |
| ATOM | 1451 | 1HA | GLY | A | 261 | 34.243 | 29.777 | 7.763 | 1.00 | 0.00 | H |
| ATOM | 1452 | 2HA | GLY | A | 261 | 32.761 | 29.785 | 6.778 | 1.00 | 0.00 | H |
| ATOM | 1453 | N | ARG | A | 262 | 31.614 | 31.607 | 8.124 | 1.00 | 0.00 | N |
| ATOM | 1454 | CA | ARG | A | 262 | 31.027 | 32.770 | 8.770 | 1.00 | 0.00 | C |
| ATOM | 1455 | C | ARG | A | 262 | 29.536 | 32.685 | 8.444 | 1.00 | 0.00 | C |
| ATOM | 1456 | O | ARG | A | 262 | 29.192 | 32.612 | 7.265 | 1.00 | 0.00 | O |
| ATOM | 1457 | CB | ARG | A | 262 | 31.701 | 34.022 | 8.176 | 1.00 | 0.00 | C |
| ATOM | 1458 | CG | ARG | A | 262 | 31.519 | 35.299 | 9.014 | 1.00 | 0.00 | C |
| ATOM | 1459 | CD | ARG | A | 262 | 32.409 | 36.460 | 8.526 | 1.00 | 0.00 | C |
| ATOM | 1460 | NE | ARG | A | 262 | 33.830 | 36.074 | 8.473 | 1.00 | 0.00 | N |
| ATOM | 1461 | CZ | ARG | A | 262 | 34.835 | 36.815 | 7.989 | 1.00 | 0.00 | C |
| ATOM | 1462 | NH1 | ARG | A | 262 | 34.864 | 38.138 | 8.095 | 1.00 | 0.00 | N1+ |
| ATOM | 1463 | NH2 | ARG | A | 262 | 35.825 | 36.172 | 7.393 | 1.00 | 0.00 | N |
| ATOM | 1464 | H | ARG | A | 262 | 31.226 | 31.367 | 7.223 | 1.00 | 0.00 | H |
| ATOM | 1465 | HA | ARG | A | 262 | 31.216 | 32.725 | 9.843 | 1.00 | 0.00 | H |
| ATOM | 1466 | 1HB | ARG | A | 262 | 32.774 | 33.850 | 8.087 | 1.00 | 0.00 | H |
| ATOM | 1467 | 2HB | ARG | A | 262 | 31.284 | 34.227 | 7.190 | 1.00 | 0.00 | H |
| ATOM | 1468 | 1HG | ARG | A | 262 | 30.481 | 35.627 | 8.958 | 1.00 | 0.00 | H |
| ATOM | 1469 | 2HG | ARG | A | 262 | 31.779 | 35.093 | 10.053 | 1.00 | 0.00 | H |
| ATOM | 1470 | 1HD | ARG | A | 262 | 32.099 | 36.760 | 7.525 | 1.00 | 0.00 | H |
| ATOM | 1471 | 2HD | ARG | A | 262 | 32.309 | 37.306 | 9.205 | 1.00 | 0.00 | H |
| ATOM | 1472 | HE | ARG | A | 262 | 34.015 | 35.155 | 8.849 | 1.00 | 0.00 | H |
| ATOM | 1473 | 1HH1 | ARG | A | 262 | 35.642 | 38.659 | 7.716 | 1.00 | 0.00 | H |
| ATOM | 1474 | 2HH1 | ARG | A | 262 | 34.108 | 38.624 | 8.556 | 1.00 | 0.00 | H |
| ATOM | 1475 | 1HH2 | ARG | A | 262 | 36.605 | 36.688 | 7.012 | 1.00 | 0.00 | H |
| ATOM | 1476 | 2HH2 | ARG | A | 262 | 35.800 | 35.165 | 7.319 | 1.00 | 0.00 | H |
| ATOM | 1477 | N | ARG | A | 263 | 28.668 | 32.603 | 9.456 | 1.00 | 0.00 | N |
| ATOM | 1478 | CA | ARG | A | 263 | 27.229 | 32.403 | 9.282 | 1.00 | 0.00 | C |
| ATOM | 1479 | C | ARG | A | 263 | 26.514 | 33.727 | 9.531 | 1.00 | 0.00 | C |
| ATOM | 1480 | O | ARG | A | 263 | 25.837 | 34.245 | 8.644 | 1.00 | 0.00 | O |
| ATOM | 1481 | CB | ARG | A | 263 | 26.698 | 31.325 | 10.238 | 1.00 | 0.00 | C |
| ATOM | 1482 | CG | ARG | A | 263 | 27.330 | 29.936 | 10.075 | 1.00 | 0.00 | C |
| ATOM | 1483 | CD | ARG | A | 263 | 27.035 | 29.280 | 8.717 | 1.00 | 0.00 | C |
| ATOM | 1484 | NE | ARG | A | 263 | 28.105 | 29.532 | 7.736 | 1.00 | 0.00 | N |
| ATOM | 1485 | CZ | ARG | A | 263 | 27.998 | 30.051 | 6.504 | 1.00 | 0.00 | C |
| ATOM | 1486 | NH1 | ARG | A | 263 | 26.877 | 30.604 | 6.064 | 1.00 | 0.00 | N1+ |
| ATOM | 1487 | NH2 | ARG | A | 263 | 29.058 | 29.990 | 5.701 | 1.00 | 0.00 | N |
| ATOM | 1488 | H | ARG | A | 263 | 28.994 | 32.678 | 10.409 | 1.00 | 0.00 | H |
| ATOM | 1489 | HA | ARG | A | 263 | 27.033 | 32.033 | 8.276 | 1.00 | 0.00 | H |
| ATOM | 1490 | 1HB | ARG | A | 263 | 26.881 | 31.630 | 11.268 | 1.00 | 0.00 | H |
| ATOM | 1491 | 2HB | ARG | A | 263 | 25.627 | 31.198 | 10.082 | 1.00 | 0.00 | H |
| ATOM | 1492 | 1HG | ARG | A | 263 | 28.413 | 30.016 | 10.168 | 1.00 | 0.00 | H |
| ATOM | 1493 | 2HG | ARG | A | 263 | 26.948 | 29.269 | 10.848 | 1.00 | 0.00 | H |
| ATOM | 1494 | 1HD | ARG | A | 263 | 26.940 | 28.202 | 8.846 | 1.00 | 0.00 | H |
| ATOM | 1495 | 2HD | ARG | A | 263 | 26.105 | 29.681 | 8.314 | 1.00 | 0.00 | H |
| ATOM | 1496 | HE | ARG | A | 263 | 29.010 | 29.259 | 8.092 | 1.00 | 0.00 | H |
| ATOM | 1497 | 1HH1 | ARG | A | 263 | 26.835 | 30.985 | 5.130 | 1.00 | 0.00 | H |
| ATOM | 1498 | 2HH1 | ARG | A | 263 | 26.065 | 30.646 | 6.664 | 1.00 | 0.00 | H |
| ATOM | 1499 | 1HH2 | ARG | A | 263 | 29.007 | 30.372 | 4.768 | 1.00 | 0.00 | H |
| ATOM | 1500 | 2HH2 | ARG | A | 263 | 29.913 | 29.561 | 6.026 | 1.00 | 0.00 | H |
| ATOM | 1501 | N | LYS | A | 264 | 26.700 | 34.292 | 10.726 | 1.00 | 0.00 | N |
| ATOM | 1502 | CA | LYS | A | 264 | 26.332 | 35.676 | 10.943 | 1.00 | 0.00 | C |
| ATOM | 1503 | C | LYS | A | 264 | 27.344 | 36.469 | 10.123 | 1.00 | 0.00 | C |
| ATOM | 1504 | O | LYS | A | 264 | 28.542 | 36.342 | 10.367 | 1.00 | 0.00 | O |
| ATOM | 1505 | CB | LYS | A | 264 | 26.378 | 36.024 | 12.447 | 1.00 | 0.00 | C |
| ATOM | 1506 | CG | LYS | A | 264 | 25.692 | 37.374 | 12.710 | 1.00 | 0.00 | C |
| ATOM | 1507 | CD | LYS | A | 264 | 25.573 | 37.788 | 14.191 | 1.00 | 0.00 | C |
| ATOM | 1508 | CE | LYS | A | 264 | 26.905 | 38.243 | 14.813 | 1.00 | 0.00 | C |
| ATOM | 1509 | NZ | LYS | A | 264 | 26.721 | 39.257 | 15.877 | 1.00 | 0.00 | N1+ |
| ATOM | 1510 | H | LYS | A | 264 | 27.099 | 33.754 | 11.482 | 1.00 | 0.00 | H |
| ATOM | 1511 | HA | LYS | A | 264 | 25.301 | 35.833 | 10.623 | 1.00 | 0.00 | H |
| ATOM | 1512 | 1HB | LYS | A | 264 | 25.863 | 35.250 | 13.015 | 1.00 | 0.00 | H |
| ATOM | 1513 | 2HB | LYS | A | 264 | 27.416 | 36.085 | 12.774 | 1.00 | 0.00 | H |
| ATOM | 1514 | 1HG | LYS | A | 264 | 26.252 | 38.168 | 12.216 | 1.00 | 0.00 | H |
| ATOM | 1515 | 2HG | LYS | A | 264 | 24.676 | 37.348 | 12.318 | 1.00 | 0.00 | H |
| ATOM | 1516 | 1HD | LYS | A | 264 | 24.872 | 38.618 | 14.281 | 1.00 | 0.00 | H |
| ATOM | 1517 | 2HD | LYS | A | 264 | 25.212 | 36.943 | 14.777 | 1.00 | 0.00 | H |
| ATOM | 1518 | 1HE | LYS | A | 264 | 27.412 | 37.385 | 15.254 | 1.00 | 0.00 | H |
| ATOM | 1519 | 2HE | LYS | A | 264 | 27.537 | 38.681 | 14.041 | 1.00 | 0.00 | H |
| ATOM | 1520 | 1HZ | LYS | A | 264 | 27.622 | 39.520 | 16.251 | 1.00 | 0.00 | H |
| ATOM | 1521 | 2HZ | LYS | A | 264 | 26.266 | 40.072 | 15.492 | 1.00 | 0.00 | H |
| ATOM | 1522 | 3HZ | LYS | A | 264 | 26.151 | 38.871 | 16.616 | 1.00 | 0.00 | H |
| ATOM | 1523 | N | GLN | A | 265 | 26.891 | 37.231 | 9.132 | 1.00 | 0.00 | N |
| ATOM | 1524 | CA | GLN | A | 265 | 27.785 | 38.053 | 8.341 | 1.00 | 0.00 | C |
| ATOM | 1525 | C | GLN | A | 265 | 27.992 | 39.361 | 9.097 | 1.00 | 0.00 | C |
| ATOM | 1526 | O | GLN | A | 265 | 27.061 | 39.849 | 9.738 | 1.00 | 0.00 | O |
| ATOM | 1527 | CB | GLN | A | 265 | 27.194 | 38.313 | 6.951 | 1.00 | 0.00 | C |
| ATOM | 1528 | CG | GLN | A | 265 | 26.746 | 37.025 | 6.249 | 1.00 | 0.00 | C |
| ATOM | 1529 | CD | GLN | A | 265 | 27.871 | 36.005 | 6.162 | 1.00 | 0.00 | C |
| ATOM | 1530 | NE2 | GLN | A | 265 | 27.617 | 34.781 | 6.600 | 1.00 | 0.00 | N |
| ATOM | 1531 | OE1 | GLN | A | 265 | 28.981 | 36.327 | 5.747 | 1.00 | 0.00 | O |
| ATOM | 1532 | H | GLN | A | 265 | 25.905 | 37.246 | 8.917 | 1.00 | 0.00 | H |
| ATOM | 1533 | HA | GLN | A | 265 | 28.727 | 37.526 | 8.192 | 1.00 | 0.00 | H |
| ATOM | 1534 | 1HB | GLN | A | 265 | 26.325 | 38.964 | 7.042 | 1.00 | 0.00 | H |
| ATOM | 1535 | 2HB | GLN | A | 265 | 27.943 | 38.793 | 6.322 | 1.00 | 0.00 | H |
| ATOM | 1536 | 1HG | GLN | A | 265 | 25.921 | 36.577 | 6.803 | 1.00 | 0.00 | H |
| ATOM | 1537 | 2HG | GLN | A | 265 | 26.418 | 37.258 | 5.236 | 1.00 | 0.00 | H |
| ATOM | 1538 | 1HE2 | GLN | A | 265 | 28.334 | 34.071 | 6.562 | 1.00 | 0.00 | H |
| ATOM | 1539 | 2HE2 | GLN | A | 265 | 26.705 | 34.557 | 6.973 | 1.00 | 0.00 | H |
| ATOM | 1540 | N | TYR | A | 266 | 29.190 | 39.932 | 8.998 | 1.00 | 0.00 | N |
| ATOM | 1541 | CA | TYR | A | 266 | 29.557 | 41.156 | 9.686 | 1.00 | 0.00 | C |
| ATOM | 1542 | C | TYR | A | 266 | 30.053 | 42.140 | 8.623 | 1.00 | 0.00 | C |
| ATOM | 1543 | O | TYR | A | 266 | 31.265 | 42.250 | 8.411 | 1.00 | 0.00 | O |
| ATOM | 1544 | CB | TYR | A | 266 | 30.673 | 40.871 | 10.700 | 1.00 | 0.00 | C |
| ATOM | 1545 | CG | TYR | A | 266 | 30.363 | 39.910 | 11.831 | 1.00 | 0.00 | C |
| ATOM | 1546 | CD1 | TYR | A | 266 | 30.196 | 40.375 | 13.149 | 1.00 | 0.00 | C |
| ATOM | 1547 | CD2 | TYR | A | 266 | 30.313 | 38.524 | 11.597 | 1.00 | 0.00 | C |
| ATOM | 1548 | CE1 | TYR | A | 266 | 30.089 | 39.469 | 14.217 | 1.00 | 0.00 | C |
| ATOM | 1549 | CE2 | TYR | A | 266 | 30.173 | 37.618 | 12.660 | 1.00 | 0.00 | C |
| ATOM | 1550 | CZ | TYR | A | 266 | 30.113 | 38.082 | 13.984 | 1.00 | 0.00 | C |
| ATOM | 1551 | OH | TYR | A | 266 | 30.116 | 37.198 | 15.024 | 1.00 | 0.00 | O |
| ATOM | 1552 | H | TYR | A | 266 | 29.904 | 39.512 | 8.420 | 1.00 | 0.00 | H |
| ATOM | 1553 | HA | TYR | A | 266 | 28.685 | 41.562 | 10.198 | 1.00 | 0.00 | H |
| ATOM | 1554 | 1HB | TYR | A | 266 | 31.531 | 40.440 | 10.184 | 1.00 | 0.00 | H |
| ATOM | 1555 | 2HB | TYR | A | 266 | 30.970 | 41.800 | 11.185 | 1.00 | 0.00 | H |
| ATOM | 1556 | HD1 | TYR | A | 266 | 30.150 | 41.436 | 13.345 | 1.00 | 0.00 | H |
| ATOM | 1557 | HD2 | TYR | A | 266 | 30.383 | 38.145 | 10.588 | 1.00 | 0.00 | H |
| ATOM | 1558 | HE1 | TYR | A | 266 | 29.988 | 39.835 | 15.228 | 1.00 | 0.00 | H |
| ATOM | 1559 | HE2 | TYR | A | 266 | 30.111 | 36.558 | 12.461 | 1.00 | 0.00 | H |
| ATOM | 1560 | HH | TYR | A | 266 | 30.133 | 36.301 | 14.682 | 1.00 | 0.00 | H |
| ATOM | 1561 | N | PRO | A | 267 | 29.152 | 42.855 | 7.935 | 1.00 | 0.00 | N |
| ATOM | 1562 | CA | PRO | A | 267 | 29.511 | 43.887 | 6.976 | 1.00 | 0.00 | C |
| ATOM | 1563 | C | PRO | A | 267 | 30.521 | 44.868 | 7.579 | 1.00 | 0.00 | C |
| ATOM | 1564 | O | PRO | A | 267 | 30.426 | 45.206 | 8.758 | 1.00 | 0.00 | O |
| ATOM | 1565 | CB | PRO | A | 267 | 28.198 | 44.599 | 6.623 | 1.00 | 0.00 | C |
| ATOM | 1566 | CG | PRO | A | 267 | 27.115 | 43.585 | 6.983 | 1.00 | 0.00 | C |
| ATOM | 1567 | CD | PRO | A | 267 | 27.723 | 42.882 | 8.192 | 1.00 | 0.00 | C |
| ATOM | 1568 | HA | PRO | A | 267 | 29.949 | 43.426 | 6.091 | 1.00 | 0.00 | H |
| ATOM | 1569 | 1HB | PRO | A | 267 | 28.112 | 45.514 | 7.208 | 1.00 | 0.00 | H |
| ATOM | 1570 | 2HB | PRO | A | 267 | 28.192 | 44.845 | 5.561 | 1.00 | 0.00 | H |
| ATOM | 1571 | 1HG | PRO | A | 267 | 26.189 | 44.110 | 7.217 | 1.00 | 0.00 | H |
| ATOM | 1572 | 2HG | PRO | A | 267 | 26.949 | 42.915 | 6.139 | 1.00 | 0.00 | H |
| ATOM | 1573 | 1HD | PRO | A | 267 | 27.352 | 41.862 | 8.291 | 1.00 | 0.00 | H |
| ATOM | 1574 | 2HD | PRO | A | 267 | 27.529 | 43.430 | 9.114 | 1.00 | 0.00 | H |
| ATOM | 1575 | N | ILE | A | 268 | 31.512 | 45.308 | 6.807 | 1.00 | 0.00 | N |
| ATOM | 1576 | CA | ILE | A | 268 | 32.443 | 46.334 | 7.260 | 1.00 | 0.00 | C |
| ATOM | 1577 | C | ILE | A | 268 | 31.731 | 47.690 | 7.203 | 1.00 | 0.00 | C |
| ATOM | 1578 | O | ILE | A | 268 | 32.121 | 48.629 | 7.895 | 1.00 | 0.00 | O |
| ATOM | 1579 | CB | ILE | A | 268 | 33.746 | 46.290 | 6.430 | 1.00 | 0.00 | C |
| ATOM | 1580 | CG1 | ILE | A | 268 | 34.351 | 44.870 | 6.451 | 1.00 | 0.00 | C |
| ATOM | 1581 | CG2 | ILE | A | 268 | 34.759 | 47.303 | 6.989 | 1.00 | 0.00 | C |
| ATOM | 1582 | CD1 | ILE | A | 268 | 35.575 | 44.712 | 5.541 | 1.00 | 0.00 | C |
| ATOM | 1583 | H | ILE | A | 268 | 31.635 | 44.930 | 5.878 | 1.00 | 0.00 | H |
| ATOM | 1584 | HA | ILE | A | 268 | 32.820 | 46.073 | 8.248 | 1.00 | 0.00 | H |
| ATOM | 1585 | HB | ILE | A | 268 | 33.535 | 46.596 | 5.405 | 1.00 | 0.00 | H |
| ATOM | 1586 | 1HG1 | ILE | A | 268 | 34.666 | 44.623 | 7.466 | 1.00 | 0.00 | H |
| ATOM | 1587 | 2HG1 | ILE | A | 268 | 33.603 | 44.150 | 6.119 | 1.00 | 0.00 | H |
| ATOM | 1588 | 1HG2 | ILE | A | 268 | 35.675 | 47.265 | 6.399 | 1.00 | 0.00 | H |
| ATOM | 1589 | 2HG2 | ILE | A | 268 | 34.337 | 48.306 | 6.939 | 1.00 | 0.00 | H |
| ATOM | 1590 | 3HG2 | ILE | A | 268 | 34.986 | 47.056 | 8.026 | 1.00 | 0.00 | H |
| ATOM | 1591 | 1HD1 | ILE | A | 268 | 35.948 | 43.690 | 5.606 | 1.00 | 0.00 | H |
| ATOM | 1592 | 2HD1 | ILE | A | 268 | 35.293 | 44.931 | 4.511 | 1.00 | 0.00 | H |
| ATOM | 1593 | 3HD1 | ILE | A | 268 | 36.355 | 45.404 | 5.858 | 1.00 | 0.00 | H |
| ATOM | 1594 | N | SER | A | 269 | 30.681 | 47.766 | 6.387 | 1.00 | 0.00 | N |
| ATOM | 1595 | CA | SER | A | 269 | 29.879 | 48.948 | 6.168 | 1.00 | 0.00 | C |
| ATOM | 1596 | C | SER | A | 269 | 28.470 | 48.482 | 5.804 | 1.00 | 0.00 | C |
| ATOM | 1597 | O | SER | A | 269 | 28.315 | 47.433 | 5.170 | 1.00 | 0.00 | O |
| ATOM | 1598 | CB | SER | A | 269 | 30.498 | 49.773 | 5.035 | 1.00 | 0.00 | C |
| ATOM | 1599 | OG | SER | A | 269 | 29.790 | 50.985 | 4.880 | 1.00 | 0.00 | O |
| ATOM | 1600 | H | SER | A | 269 | 30.390 | 46.954 | 5.861 | 1.00 | 0.00 | H |
| ATOM | 1601 | HA | SER | A | 269 | 29.869 | 49.554 | 7.074 | 1.00 | 0.00 | H |
| ATOM | 1602 | 1HB | SER | A | 269 | 31.539 | 49.993 | 5.273 | 1.00 | 0.00 | H |
| ATOM | 1603 | 2HB | SER | A | 269 | 30.450 | 49.207 | 4.105 | 1.00 | 0.00 | H |
| ATOM | 1604 | HG | SER | A | 269 | 30.183 | 51.497 | 4.169 | 1.00 | 0.00 | H |
| ATOM | 1605 | N | LEU | A | 270 | 27.464 | 49.251 | 6.209 | 1.00 | 0.00 | N |
| ATOM | 1606 | CA | LEU | A | 270 | 26.055 | 48.970 | 6.010 | 1.00 | 0.00 | C |
| ATOM | 1607 | C | LEU | A | 270 | 25.412 | 50.219 | 5.421 | 1.00 | 0.00 | C |
| ATOM | 1608 | O | LEU | A | 270 | 25.711 | 51.328 | 5.861 | 1.00 | 0.00 | O |
| ATOM | 1609 | CB | LEU | A | 270 | 25.418 | 48.644 | 7.369 | 1.00 | 0.00 | C |
| ATOM | 1610 | CG | LEU | A | 270 | 23.879 | 48.580 | 7.341 | 1.00 | 0.00 | C |
| ATOM | 1611 | CD1 | LEU | A | 270 | 23.363 | 47.417 | 6.484 | 1.00 | 0.00 | C |
| ATOM | 1612 | CD2 | LEU | A | 270 | 23.364 | 48.418 | 8.770 | 1.00 | 0.00 | C |
| ATOM | 1613 | H | LEU | A | 270 | 27.653 | 50.113 | 6.701 | 1.00 | 0.00 | H |
| ATOM | 1614 | HA | LEU | A | 270 | 25.945 | 48.138 | 5.313 | 1.00 | 0.00 | H |
| ATOM | 1615 | 1HB | LEU | A | 270 | 25.774 | 47.673 | 7.714 | 1.00 | 0.00 | H |
| ATOM | 1616 | 2HB | LEU | A | 270 | 25.694 | 49.410 | 8.093 | 1.00 | 0.00 | H |
| ATOM | 1617 | HG | LEU | A | 270 | 23.483 | 49.514 | 6.942 | 1.00 | 0.00 | H |
| ATOM | 1618 | 1HD1 | LEU | A | 270 | 22.273 | 47.413 | 6.494 | 1.00 | 0.00 | H |
| ATOM | 1619 | 2HD1 | LEU | A | 270 | 23.716 | 47.535 | 5.459 | 1.00 | 0.00 | H |
| ATOM | 1620 | 3HD1 | LEU | A | 270 | 23.734 | 46.475 | 6.888 | 1.00 | 0.00 | H |
| ATOM | 1621 | 1HD2 | LEU | A | 270 | 22.275 | 48.372 | 8.761 | 1.00 | 0.00 | H |
| ATOM | 1622 | 2HD2 | LEU | A | 270 | 23.763 | 47.499 | 9.199 | 1.00 | 0.00 | H |
| ATOM | 1623 | 3HD2 | LEU | A | 270 | 23.686 | 49.268 | 9.371 | 1.00 | 0.00 | H |
| ATOM | 1624 | N | VAL | A | 271 | 24.498 | 50.036 | 4.473 | 1.00 | 0.00 | N |
| ATOM | 1625 | CA | VAL | A | 271 | 23.711 | 51.117 | 3.914 | 1.00 | 0.00 | C |
| ATOM | 1626 | C | VAL | A | 271 | 22.229 | 50.773 | 4.083 | 1.00 | 0.00 | C |
| ATOM | 1627 | O | VAL | A | 271 | 21.823 | 49.632 | 3.853 | 1.00 | 0.00 | O |
| ATOM | 1628 | CB | VAL | A | 271 | 24.085 | 51.329 | 2.439 | 1.00 | 0.00 | C |
| ATOM | 1629 | CG1 | VAL | A | 271 | 23.370 | 52.577 | 1.902 | 1.00 | 0.00 | C |
| ATOM | 1630 | CG2 | VAL | A | 271 | 25.606 | 51.430 | 2.229 | 1.00 | 0.00 | C |
| ATOM | 1631 | H | VAL | A | 271 | 24.325 | 49.110 | 4.109 | 1.00 | 0.00 | H |
| ATOM | 1632 | HA | VAL | A | 271 | 23.974 | 52.052 | 4.408 | 1.00 | 0.00 | H |
| ATOM | 1633 | HB | VAL | A | 271 | 23.847 | 50.431 | 1.870 | 1.00 | 0.00 | H |
| ATOM | 1634 | 1HG1 | VAL | A | 271 | 23.636 | 52.728 | 0.856 | 1.00 | 0.00 | H |
| ATOM | 1635 | 2HG1 | VAL | A | 271 | 22.291 | 52.443 | 1.986 | 1.00 | 0.00 | H |
| ATOM | 1636 | 3HG1 | VAL | A | 271 | 23.673 | 53.449 | 2.482 | 1.00 | 0.00 | H |
| ATOM | 1637 | 1HG2 | VAL | A | 271 | 25.818 | 51.580 | 1.171 | 1.00 | 0.00 | H |
| ATOM | 1638 | 2HG2 | VAL | A | 271 | 25.997 | 52.272 | 2.801 | 1.00 | 0.00 | H |
| ATOM | 1639 | 3HG2 | VAL | A | 271 | 26.081 | 50.509 | 2.568 | 1.00 | 0.00 | H |
| ATOM | 1640 | N | LEU | A | 272 | 21.424 | 51.756 | 4.471 | 1.00 | 0.00 | N |
| ATOM | 1641 | CA | LEU | A | 272 | 19.987 | 51.629 | 4.621 | 1.00 | 0.00 | C |
| ATOM | 1642 | C | LEU | A | 272 | 19.316 | 52.516 | 3.576 | 1.00 | 0.00 | C |
| ATOM | 1643 | O | LEU | A | 272 | 19.849 | 53.562 | 3.210 | 1.00 | 0.00 | O |
| ATOM | 1644 | CB | LEU | A | 272 | 19.562 | 52.039 | 6.037 | 1.00 | 0.00 | C |
| ATOM | 1645 | CG | LEU | A | 272 | 20.189 | 51.192 | 7.156 | 1.00 | 0.00 | C |
| ATOM | 1646 | CD1 | LEU | A | 272 | 19.724 | 51.728 | 8.516 | 1.00 | 0.00 | C |
| ATOM | 1647 | CD2 | LEU | A | 272 | 19.798 | 49.714 | 7.037 | 1.00 | 0.00 | C |
| ATOM | 1648 | H | LEU | A | 272 | 21.805 | 52.667 | 4.683 | 1.00 | 0.00 | H |
| ATOM | 1649 | HA | LEU | A | 272 | 19.699 | 50.585 | 4.495 | 1.00 | 0.00 | H |
| ATOM | 1650 | 1HB | LEU | A | 272 | 19.854 | 53.073 | 6.219 | 1.00 | 0.00 | H |
| ATOM | 1651 | 2HB | LEU | A | 272 | 18.481 | 51.945 | 6.134 | 1.00 | 0.00 | H |
| ATOM | 1652 | HG | LEU | A | 272 | 21.276 | 51.238 | 7.082 | 1.00 | 0.00 | H |
| ATOM | 1653 | 1HD1 | LEU | A | 272 | 20.166 | 51.130 | 9.313 | 1.00 | 0.00 | H |
| ATOM | 1654 | 2HD1 | LEU | A | 272 | 20.038 | 52.766 | 8.624 | 1.00 | 0.00 | H |
| ATOM | 1655 | 3HD1 | LEU | A | 272 | 18.638 | 51.669 | 8.578 | 1.00 | 0.00 | H |
| ATOM | 1656 | 1HD2 | LEU | A | 272 | 20.261 | 49.149 | 7.846 | 1.00 | 0.00 | H |
| ATOM | 1657 | 2HD2 | LEU | A | 272 | 18.715 | 49.618 | 7.101 | 1.00 | 0.00 | H |
| ATOM | 1658 | 3HD2 | LEU | A | 272 | 20.141 | 49.323 | 6.079 | 1.00 | 0.00 | H |
| ATOM | 1659 | N | ALA | A | 273 | 18.153 | 52.102 | 3.083 | 1.00 | 0.00 | N |
| ATOM | 1660 | CA | ALA | A | 273 | 17.450 | 52.796 | 2.019 | 1.00 | 0.00 | C |
| ATOM | 1661 | C | ALA | A | 273 | 15.983 | 52.369 | 2.080 | 1.00 | 0.00 | C |
| ATOM | 1662 | O | ALA | A | 273 | 15.703 | 51.253 | 2.519 | 1.00 | 0.00 | O |
| ATOM | 1663 | CB | ALA | A | 273 | 18.065 | 52.389 | 0.678 | 1.00 | 0.00 | C |
| ATOM | 1664 | H | ALA | A | 273 | 17.718 | 51.268 | 3.449 | 1.00 | 0.00 | H |
| ATOM | 1665 | HA | ALA | A | 273 | 17.530 | 53.872 | 2.171 | 1.00 | 0.00 | H |
| ATOM | 1666 | 1HB | ALA | A | 273 | 17.547 | 52.903 | −0.131 | 1.00 | 0.00 | H |
| ATOM | 1667 | 2HB | ALA | A | 273 | 19.120 | 52.663 | 0.664 | 1.00 | 0.00 | H |
| ATOM | 1668 | 3HB | ALA | A | 273 | 17.969 | 51.312 | 0.545 | 1.00 | 0.00 | H |
| ATOM | 1669 | N | PRO | A | 274 | 15.030 | 53.206 | 1.655 | 1.00 | 0.00 | N |
| ATOM | 1670 | CA | PRO | A | 274 | 13.629 | 52.972 | 1.968 | 1.00 | 0.00 | C |
| ATOM | 1671 | C | PRO | A | 274 | 13.037 | 51.885 | 1.076 | 1.00 | 0.00 | C |
| ATOM | 1672 | O | PRO | A | 274 | 12.124 | 51.165 | 1.476 | 1.00 | 0.00 | O |
| ATOM | 1673 | CB | PRO | A | 274 | 12.934 | 54.319 | 1.745 | 1.00 | 0.00 | C |
| ATOM | 1674 | CG | PRO | A | 274 | 13.853 | 55.052 | 0.768 | 1.00 | 0.00 | C |
| ATOM | 1675 | CD | PRO | A | 274 | 15.229 | 54.587 | 1.236 | 1.00 | 0.00 | C |
| ATOM | 1676 | HA | PRO | A | 274 | 13.535 | 52.672 | 3.011 | 1.00 | 0.00 | H |
| ATOM | 1677 | 1HB | PRO | A | 274 | 11.941 | 54.151 | 1.327 | 1.00 | 0.00 | H |
| ATOM | 1678 | 2HB | PRO | A | 274 | 12.844 | 54.844 | 2.696 | 1.00 | 0.00 | H |
| ATOM | 1679 | 1HG | PRO | A | 274 | 13.619 | 54.746 | −0.252 | 1.00 | 0.00 | H |
| ATOM | 1680 | 2HG | PRO | A | 274 | 13.705 | 56.127 | 0.866 | 1.00 | 0.00 | H |
| ATOM | 1681 | 1HD | PRO | A | 274 | 15.589 | 55.175 | 2.081 | 1.00 | 0.00 | H |
| ATOM | 1682 | 2HD | PRO | A | 274 | 15.963 | 54.621 | 0.432 | 1.00 | 0.00 | H |
| ATOM | 1683 | N | THR | A | 275 | 13.560 | 51.763 | −0.140 | 1.00 | 0.00 | N |
| ATOM | 1684 | CA | THR | A | 275 | 12.913 | 51.025 | −1.200 | 1.00 | 0.00 | C |
| ATOM | 1685 | C | THR | A | 275 | 13.906 | 50.045 | −1.818 | 1.00 | 0.00 | C |
| ATOM | 1686 | O | THR | A | 275 | 15.108 | 50.310 | −1.898 | 1.00 | 0.00 | O |
| ATOM | 1687 | CB | THR | A | 275 | 12.392 | 52.027 | −2.241 | 1.00 | 0.00 | C |
| ATOM | 1688 | CG2 | THR | A | 275 | 11.259 | 52.880 | −1.661 | 1.00 | 0.00 | C |
| ATOM | 1689 | OG1 | THR | A | 275 | 13.445 | 52.870 | −2.668 | 1.00 | 0.00 | O |
| ATOM | 1690 | H | THR | A | 275 | 14.446 | 52.198 | −0.354 | 1.00 | 0.00 | H |
| ATOM | 1691 | HA | THR | A | 275 | 12.046 | 50.499 | −0.800 | 1.00 | 0.00 | H |
| ATOM | 1692 | HB | THR | A | 275 | 12.032 | 51.487 | −3.117 | 1.00 | 0.00 | H |
| ATOM | 1693 | 1HG2 | THR | A | 275 | 10.907 | 53.581 | −2.418 | 1.00 | 0.00 | H |
| ATOM | 1694 | 2HG2 | THR | A | 275 | 10.436 | 52.233 | −1.355 | 1.00 | 0.00 | H |
| ATOM | 1695 | 3HG2 | THR | A | 275 | 11.626 | 53.433 | −0.797 | 1.00 | 0.00 | H |
| ATOM | 1696 | HG1 | THR | A | 275 | 13.113 | 53.494 | −3.318 | 1.00 | 0.00 | H |
| ATOM | 1697 | N | ARG | A | 276 | 13.393 | 48.905 | −2.292 | 1.00 | 0.00 | N |
| ATOM | 1698 | CA | ARG | A | 276 | 14.191 | 47.935 | −3.017 | 1.00 | 0.00 | C |
| ATOM | 1699 | C | ARG | A | 276 | 14.982 | 48.614 | −4.136 | 1.00 | 0.00 | C |
| ATOM | 1700 | O | ARG | A | 276 | 16.090 | 48.191 | −4.441 | 1.00 | 0.00 | O |
| ATOM | 1701 | CB | ARG | A | 276 | 13.291 | 46.824 | −3.584 | 1.00 | 0.00 | C |
| ATOM | 1702 | CG | ARG | A | 276 | 14.081 | 45.908 | −4.530 | 1.00 | 0.00 | C |
| ATOM | 1703 | CD | ARG | A | 276 | 13.328 | 44.604 | −4.820 | 1.00 | 0.00 | C |
| ATOM | 1704 | NE | ARG | A | 276 | 13.890 | 43.843 | −5.950 | 1.00 | 0.00 | N |
| ATOM | 1705 | CZ | ARG | A | 276 | 15.146 | 43.455 | −6.184 | 1.00 | 0.00 | C |
| ATOM | 1706 | NH1 | ARG | A | 276 | 16.072 | 43.621 | −5.241 | 1.00 | 0.00 | N1+ |
| ATOM | 1707 | NH2 | ARG | A | 276 | 15.481 | 42.919 | −7.352 | 1.00 | 0.00 | N |
| ATOM | 1708 | H | ARG | A | 276 | 12.416 | 48.693 | −2.150 | 1.00 | 0.00 | H |
| ATOM | 1709 | HA | ARG | A | 276 | 14.856 | 47.420 | −2.324 | 1.00 | 0.00 | H |
| ATOM | 1710 | 1HB | ARG | A | 276 | 12.896 | 46.223 | −2.764 | 1.00 | 0.00 | H |
| ATOM | 1711 | 2HB | ARG | A | 276 | 12.466 | 47.271 | −4.137 | 1.00 | 0.00 | H |
| ATOM | 1712 | 1HG | ARG | A | 276 | 14.251 | 46.422 | −5.476 | 1.00 | 0.00 | H |
| ATOM | 1713 | 2HG | ARG | A | 276 | 15.039 | 45.656 | −4.077 | 1.00 | 0.00 | H |
| ATOM | 1714 | 1HD | ARG | A | 276 | 13.365 | 43.960 | −3.942 | 1.00 | 0.00 | H |
| ATOM | 1715 | 2HD | ARG | A | 276 | 12.290 | 44.829 | −5.062 | 1.00 | 0.00 | H |
| ATOM | 1716 | HE | ARG | A | 276 | 13.160 | 43.613 | −6.609 | 1.00 | 0.00 | H |
| ATOM | 1717 | 1HH1 | ARG | A | 276 | 17.024 | 43.330 | −5.410 | 1.00 | 0.00 | H |
| ATOM | 1718 | 2HH1 | ARG | A | 276 | 15.821 | 44.038 | −4.355 | 1.00 | 0.00 | H |
| ATOM | 1719 | 1HH2 | ARG | A | 276 | 16.434 | 42.629 | −7.520 | 1.00 | 0.00 | H |
| ATOM | 1720 | 2HH2 | ARG | A | 276 | 14.783 | 42.802 | −8.072 | 1.00 | 0.00 | H |
| ATOM | 1721 | N | GLU | A | 277 | 14.393 | 49.613 | −4.788 | 1.00 | 0.00 | N |
| ATOM | 1722 | CA | GLU | A | 277 | 15.024 | 50.241 | −5.924 | 1.00 | 0.00 | C |
| ATOM | 1723 | C | GLU | A | 277 | 16.305 | 50.974 | −5.517 | 1.00 | 0.00 | C |
| ATOM | 1724 | O | GLU | A | 277 | 17.351 | 50.762 | −6.127 | 1.00 | 0.00 | O |
| ATOM | 1725 | CB | GLU | A | 277 | 14.053 | 51.204 | −6.586 | 1.00 | 0.00 | C |
| ATOM | 1726 | CG | GLU | A | 277 | 12.760 | 50.565 | −7.095 | 1.00 | 0.00 | C |
| ATOM | 1727 | CD | GLU | A | 277 | 12.041 | 51.522 | −8.023 | 1.00 | 0.00 | C |
| ATOM | 1728 | OE1 | GLU | A | 277 | 12.337 | 52.732 | −8.009 | 1.00 | 0.00 | O |
| ATOM | 1729 | OE2 | GLU | A | 277 | 11.172 | 51.061 | −8.788 | 1.00 | 0.00 | O1− |
| ATOM | 1730 | H | GLU | A | 277 | 13.487 | 49.946 | −4.491 | 1.00 | 0.00 | H |
| ATOM | 1731 | HA | GLU | A | 277 | 15.240 | 49.488 | −6.682 | 1.00 | 0.00 | H |
| ATOM | 1732 | 1HB | GLU | A | 277 | 13.762 | 51.975 | −5.873 | 1.00 | 0.00 | H |
| ATOM | 1733 | 2HB | GLU | A | 277 | 14.533 | 51.669 | −7.447 | 1.00 | 0.00 | H |
| ATOM | 1734 | 1HG | GLU | A | 277 | 12.995 | 49.649 | −7.637 | 1.00 | 0.00 | H |
| ATOM | 1735 | 2HG | GLU | A | 277 | 12.113 | 50.330 | −6.250 | 1.00 | 0.00 | H |
| ATOM | 1736 | N | LEU | A | 278 | 16.224 | 51.868 | −4.528 | 1.00 | 0.00 | N |
| ATOM | 1737 | CA | LEU | A | 278 | 17.405 | 52.612 | −4.110 | 1.00 | 0.00 | C |
| ATOM | 1738 | C | LEU | A | 278 | 18.412 | 51.621 | −3.526 | 1.00 | 0.00 | C |
| ATOM | 1739 | O | LEU | A | 278 | 19.616 | 51.735 | −3.751 | 1.00 | 0.00 | O |
| ATOM | 1740 | CB | LEU | A | 278 | 17.038 | 53.724 | −3.114 | 1.00 | 0.00 | C |
| ATOM | 1741 | CG | LEU | A | 278 | 18.201 | 54.695 | −2.835 | 1.00 | 0.00 | C |
| ATOM | 1742 | CD1 | LEU | A | 278 | 18.479 | 55.613 | −4.032 | 1.00 | 0.00 | C |
| ATOM | 1743 | CD2 | LEU | A | 278 | 17.866 | 55.582 | −1.635 | 1.00 | 0.00 | C |
| ATOM | 1744 | H | LEU | A | 278 | 15.342 | 52.031 | −4.065 | 1.00 | 0.00 | H |
| ATOM | 1745 | HA | LEU | A | 278 | 17.799 | 53.175 | −4.956 | 1.00 | 0.00 | H |
| ATOM | 1746 | 1HB | LEU | A | 278 | 16.208 | 54.309 | −3.511 | 1.00 | 0.00 | H |
| ATOM | 1747 | 2HB | LEU | A | 278 | 16.746 | 53.279 | −2.163 | 1.00 | 0.00 | H |
| ATOM | 1748 | HG | LEU | A | 278 | 19.108 | 54.127 | −2.628 | 1.00 | 0.00 | H |
| ATOM | 1749 | 1HD1 | LEU | A | 278 | 19.306 | 56.282 | −3.793 | 1.00 | 0.00 | H |
| ATOM | 1750 | 2HD1 | LEU | A | 278 | 18.740 | 55.009 | −4.901 | 1.00 | 0.00 | H |
| ATOM | 1751 | 3HD1 | LEU | A | 278 | 17.588 | 56.202 | −4.253 | 1.00 | 0.00 | H |
| ATOM | 1752 | 1HD2 | LEU | A | 278 | 18.694 | 56.266 | −1.445 | 1.00 | 0.00 | H |
| ATOM | 1753 | 2HD2 | LEU | A | 278 | 16.964 | 56.155 | −1.846 | 1.00 | 0.00 | H |
| ATOM | 1754 | 3HD2 | LEU | A | 278 | 17.702 | 54.958 | −0.756 | 1.00 | 0.00 | H |
| ATOM | 1755 | N | ALA | A | 279 | 17.910 | 50.622 | −2.794 | 1.00 | 0.00 | N |
| ATOM | 1756 | CA | ALA | A | 279 | 18.759 | 49.596 | −2.224 | 1.00 | 0.00 | C |
| ATOM | 1757 | C | ALA | A | 279 | 19.503 | 48.828 | −3.324 | 1.00 | 0.00 | C |
| ATOM | 1758 | O | ALA | A | 279 | 20.635 | 48.391 | −3.125 | 1.00 | 0.00 | O |
| ATOM | 1759 | CB | ALA | A | 279 | 17.927 | 48.651 | −1.353 | 1.00 | 0.00 | C |
| ATOM | 1760 | H | ALA | A | 279 | 16.915 | 50.570 | −2.626 | 1.00 | 0.00 | H |
| ATOM | 1761 | HA | ALA | A | 279 | 19.450 | 50.049 | −1.513 | 1.00 | 0.00 | H |
| ATOM | 1762 | 1HB | ALA | A | 279 | 18.574 | 47.883 | −0.929 | 1.00 | 0.00 | H |
| ATOM | 1763 | 2HB | ALA | A | 279 | 17.459 | 49.216 | −0.547 | 1.00 | 0.00 | H |
| ATOM | 1764 | 3HB | ALA | A | 279 | 17.155 | 48.180 | −1.961 | 1.00 | 0.00 | H |
| ATOM | 1765 | N | VAL | A | 280 | 18.854 | 48.620 | −4.470 | 1.00 | 0.00 | N |
| ATOM | 1766 | CA | VAL | A | 280 | 19.473 | 48.004 | −5.628 | 1.00 | 0.00 | C |
| ATOM | 1767 | C | VAL | A | 280 | 20.518 | 48.956 | −6.208 | 1.00 | 0.00 | C |
| ATOM | 1768 | O | VAL | A | 280 | 21.645 | 48.516 | −6.421 | 1.00 | 0.00 | O |
| ATOM | 1769 | CB | VAL | A | 280 | 18.407 | 47.539 | −6.640 | 1.00 | 0.00 | C |
| ATOM | 1770 | CG1 | VAL | A | 280 | 18.967 | 47.287 | −8.046 | 1.00 | 0.00 | C |
| ATOM | 1771 | CG2 | VAL | A | 280 | 17.779 | 46.224 | −6.157 | 1.00 | 0.00 | C |
| ATOM | 1772 | H | VAL | A | 280 | 17.886 | 48.895 | −4.559 | 1.00 | 0.00 | H |
| ATOM | 1773 | HA | VAL | A | 280 | 19.847 | 47.016 | −5.358 | 1.00 | 0.00 | H |
| ATOM | 1774 | HB | VAL | A | 280 | 17.633 | 48.301 | −6.731 | 1.00 | 0.00 | H |
| ATOM | 1775 | 1HG1 | VAL | A | 280 | 18.162 | 46.962 | −8.706 | 1.00 | 0.00 | H |
| ATOM | 1776 | 2HG1 | VAL | A | 280 | 19.405 | 48.207 | −8.434 | 1.00 | 0.00 | H |
| ATOM | 1777 | 3HG1 | VAL | A | 280 | 19.733 | 46.512 | −8.000 | 1.00 | 0.00 | H |
| ATOM | 1778 | 1HG2 | VAL | A | 280 | 17.026 | 45.895 | −6.873 | 1.00 | 0.00 | H |
| ATOM | 1779 | 2HG2 | VAL | A | 280 | 18.553 | 45.461 | −6.069 | 1.00 | 0.00 | H |
| ATOM | 1780 | 3HG2 | VAL | A | 280 | 17.311 | 46.380 | −5.184 | 1.00 | 0.00 | H |
| ATOM | 1781 | N | GLN | A | 281 | 20.166 | 50.224 | −6.472 | 1.00 | 0.00 | N |
| ATOM | 1782 | CA | GLN | A | 281 | 21.088 | 51.189 | −7.071 | 1.00 | 0.00 | C |
| ATOM | 1783 | C | GLN | A | 281 | 22.471 | 51.080 | −6.439 | 1.00 | 0.00 | C |
| ATOM | 1784 | O | GLN | A | 281 | 23.470 | 50.881 | −7.122 | 1.00 | 0.00 | O |
| ATOM | 1785 | CB | GLN | A | 281 | 20.627 | 52.645 | −6.899 | 1.00 | 0.00 | C |
| ATOM | 1786 | CG | GLN | A | 281 | 19.431 | 53.044 | −7.762 | 1.00 | 0.00 | C |
| ATOM | 1787 | CD | GLN | A | 281 | 19.547 | 54.513 | −8.153 | 1.00 | 0.00 | C |
| ATOM | 1788 | NE2 | GLN | A | 281 | 18.725 | 55.397 | −7.602 | 1.00 | 0.00 | N |
| ATOM | 1789 | OE1 | GLN | A | 281 | 20.404 | 54.871 | −8.948 | 1.00 | 0.00 | O |
| ATOM | 1790 | H | GLN | A | 281 | 19.233 | 50.544 | −6.255 | 1.00 | 0.00 | H |
| ATOM | 1791 | HA | GLN | A | 281 | 21.172 | 50.996 | −8.140 | 1.00 | 0.00 | H |
| ATOM | 1792 | 1HB | GLN | A | 281 | 20.336 | 52.814 | −5.863 | 1.00 | 0.00 | H |
| ATOM | 1793 | 2HB | GLN | A | 281 | 21.442 | 53.318 | −7.162 | 1.00 | 0.00 | H |
| ATOM | 1794 | 1HG | GLN | A | 281 | 19.412 | 52.431 | −8.664 | 1.00 | 0.00 | H |
| ATOM | 1795 | 2HG | GLN | A | 281 | 18.509 | 52.891 | −7.200 | 1.00 | 0.00 | H |
| ATOM | 1796 | 1HE2 | GLN | A | 281 | 18.792 | 56.373 | −7.853 | 1.00 | 0.00 | H |
| ATOM | 1797 | 2HE2 | GLN | A | 281 | 18.033 | 55.094 | −6.932 | 1.00 | 0.00 | H |
| ATOM | 1798 | N | ILE | A | 282 | 22.509 | 51.243 | −5.122 | 1.00 | 0.00 | N |
| ATOM | 1799 | CA | ILE | A | 282 | 23.742 | 51.381 | −4.374 | 1.00 | 0.00 | C |
| ATOM | 1800 | C | ILE | A | 282 | 24.575 | 50.098 | −4.512 | 1.00 | 0.00 | C |
| ATOM | 1801 | O | ILE | A | 282 | 25.804 | 50.132 | −4.476 | 1.00 | 0.00 | O |
| ATOM | 1802 | CB | ILE | A | 282 | 23.368 | 51.706 | −2.917 | 1.00 | 0.00 | C |
| ATOM | 1803 | CG1 | ILE | A | 282 | 22.624 | 53.055 | −2.844 | 1.00 | 0.00 | C |
| ATOM | 1804 | CG2 | ILE | A | 282 | 24.610 | 51.754 | −2.022 | 1.00 | 0.00 | C |
| ATOM | 1805 | CD1 | ILE | A | 282 | 21.823 | 53.232 | −1.551 | 1.00 | 0.00 | C |
| ATOM | 1806 | H | ILE | A | 282 | 21.650 | 51.277 | −4.592 | 1.00 | 0.00 | H |
| ATOM | 1807 | HA | ILE | A | 282 | 24.283 | 52.261 | −4.723 | 1.00 | 0.00 | H |
| ATOM | 1808 | HB | ILE | A | 282 | 22.750 | 50.904 | −2.512 | 1.00 | 0.00 | H |
| ATOM | 1809 | 1HG1 | ILE | A | 282 | 23.344 | 53.872 | −2.900 | 1.00 | 0.00 | H |
| ATOM | 1810 | 2HG1 | ILE | A | 282 | 21.925 | 53.131 | −3.677 | 1.00 | 0.00 | H |
| ATOM | 1811 | 1HG2 | ILE | A | 282 | 24.313 | 51.985 | −0.999 | 1.00 | 0.00 | H |
| ATOM | 1812 | 2HG2 | ILE | A | 282 | 25.112 | 50.786 | −2.044 | 1.00 | 0.00 | H |
| ATOM | 1813 | 3HG2 | ILE | A | 282 | 25.291 | 52.524 | −2.385 | 1.00 | 0.00 | H |
| ATOM | 1814 | 1HD1 | ILE | A | 282 | 21.324 | 54.201 | −1.562 | 1.00 | 0.00 | H |
| ATOM | 1815 | 2HD1 | ILE | A | 282 | 21.078 | 52.440 | −1.473 | 1.00 | 0.00 | H |
| ATOM | 1816 | 3HD1 | ILE | A | 282 | 22.498 | 53.181 | −0.696 | 1.00 | 0.00 | H |
| ATOM | 1817 | N | TYR | A | 283 | 23.908 | 48.955 | −4.642 | 1.00 | 0.00 | N |
| ATOM | 1818 | CA | TYR | A | 283 | 24.547 | 47.658 | −4.729 | 1.00 | 0.00 | C |
| ATOM | 1819 | C | TYR | A | 283 | 25.065 | 47.413 | −6.149 | 1.00 | 0.00 | C |
| ATOM | 1820 | O | TYR | A | 283 | 26.177 | 46.906 | −6.320 | 1.00 | 0.00 | O |
| ATOM | 1821 | CB | TYR | A | 283 | 23.558 | 46.587 | −4.255 | 1.00 | 0.00 | C |
| ATOM | 1822 | CG | TYR | A | 283 | 23.778 | 45.198 | −4.819 | 1.00 | 0.00 | C |
| ATOM | 1823 | CD1 | TYR | A | 283 | 24.805 | 44.368 | −4.335 | 1.00 | 0.00 | C |
| ATOM | 1824 | CD2 | TYR | A | 283 | 22.946 | 44.722 | −5.846 | 1.00 | 0.00 | C |
| ATOM | 1825 | CE1 | TYR | A | 283 | 25.002 | 43.088 | −4.882 | 1.00 | 0.00 | C |
| ATOM | 1826 | CE2 | TYR | A | 283 | 23.122 | 43.435 | −6.373 | 1.00 | 0.00 | C |
| ATOM | 1827 | CZ | TYR | A | 283 | 24.165 | 42.612 | −5.909 | 1.00 | 0.00 | C |
| ATOM | 1828 | OH | TYR | A | 283 | 24.367 | 41.377 | −6.453 | 1.00 | 0.00 | O |
| ATOM | 1829 | H | TYR | A | 283 | 22.899 | 48.962 | −4.686 | 1.00 | 0.00 | H |
| ATOM | 1830 | HA | TYR | A | 283 | 25.331 | 47.588 | −3.976 | 1.00 | 0.00 | H |
| ATOM | 1831 | 1HB | TYR | A | 283 | 23.618 | 46.491 | −3.171 | 1.00 | 0.00 | H |
| ATOM | 1832 | 2HB | TYR | A | 283 | 22.545 | 46.876 | −4.538 | 1.00 | 0.00 | H |
| ATOM | 1833 | HD1 | TYR | A | 283 | 25.448 | 44.712 | −3.539 | 1.00 | 0.00 | H |
| ATOM | 1834 | HD2 | TYR | A | 283 | 22.160 | 45.349 | −6.240 | 1.00 | 0.00 | H |
| ATOM | 1835 | HE1 | TYR | A | 283 | 25.801 | 42.461 | −4.515 | 1.00 | 0.00 | H |
| ATOM | 1836 | HE2 | TYR | A | 283 | 22.455 | 43.069 | −7.139 | 1.00 | 0.00 | H |
| ATOM | 1837 | HH | TYR | A | 283 | 23.710 | 41.216 | −7.135 | 1.00 | 0.00 | H |
| ATOM | 1838 | N | GLU | A | 284 | 24.262 | 47.743 | −7.161 | 1.00 | 0.00 | N |
| ATOM | 1839 | CA | GLU | A | 284 | 24.687 | 47.674 | −8.547 | 1.00 | 0.00 | C |
| ATOM | 1840 | C | GLU | A | 284 | 25.887 | 48.609 | −8.743 | 1.00 | 0.00 | C |
| ATOM | 1841 | O | GLU | A | 284 | 26.858 | 48.246 | −9.408 | 1.00 | 0.00 | O |
| ATOM | 1842 | CB | GLU | A | 284 | 23.527 | 48.053 | −9.481 | 1.00 | 0.00 | C |
| ATOM | 1843 | CG | GLU | A | 284 | 22.327 | 47.093 | −9.389 | 1.00 | 0.00 | C |
| ATOM | 1844 | CD | GLU | A | 284 | 22.666 | 45.688 | −9.839 | 1.00 | 0.00 | C |
| ATOM | 1845 | OE1 | GLU | A | 284 | 22.332 | 44.727 | −9.116 | 1.00 | 0.00 | O |
| ATOM | 1846 | OE2 | GLU | A | 284 | 23.365 | 45.544 | −10.861 | 1.00 | 0.00 | O1− |
| ATOM | 1847 | H | GLU | A | 284 | 23.319 | 48.055 | −6.977 | 1.00 | 0.00 | H |
| ATOM | 1848 | HA | GLU | A | 284 | 24.932 | 46.643 | −8.802 | 1.00 | 0.00 | H |
| ATOM | 1849 | 1HB | GLU | A | 284 | 23.167 | 49.050 | −9.227 | 1.00 | 0.00 | H |
| ATOM | 1850 | 2HB | GLU | A | 284 | 23.874 | 48.044 | −10.514 | 1.00 | 0.00 | H |
| ATOM | 1851 | 1HG | GLU | A | 284 | 21.984 | 47.038 | −8.356 | 1.00 | 0.00 | H |
| ATOM | 1852 | 2HG | GLU | A | 284 | 21.519 | 47.460 | −10.021 | 1.00 | 0.00 | H |
| ATOM | 1853 | N | GLU | A | 285 | 25.808 | 49.804 | −8.157 | 1.00 | 0.00 | N |
| ATOM | 1854 | CA | GLU | A | 285 | 26.842 | 50.816 | −8.227 | 1.00 | 0.00 | C |
| ATOM | 1855 | C | GLU | A | 285 | 28.108 | 50.279 | −7.558 | 1.00 | 0.00 | C |
| ATOM | 1856 | O | GLU | A | 285 | 29.181 | 50.265 | −8.161 | 1.00 | 0.00 | O |
| ATOM | 1857 | CB | GLU | A | 285 | 26.347 | 52.107 | −7.554 | 1.00 | 0.00 | C |
| ATOM | 1858 | CG | GLU | A | 285 | 27.281 | 53.309 | −7.766 | 1.00 | 0.00 | C |
| ATOM | 1859 | CD | GLU | A | 285 | 27.335 | 53.738 | −9.217 | 1.00 | 0.00 | C |
| ATOM | 1860 | OE1 | GLU | A | 285 | 26.439 | 53.347 | −9.990 | 1.00 | 0.00 | O |
| ATOM | 1861 | OE2 | GLU | A | 285 | 28.310 | 54.402 | −9.628 | 1.00 | 0.00 | O1− |
| ATOM | 1862 | H | GLU | A | 285 | 24.987 | 50.052 | −7.623 | 1.00 | 0.00 | H |
| ATOM | 1863 | HA | GLU | A | 285 | 27.018 | 51.085 | −9.268 | 1.00 | 0.00 | H |
| ATOM | 1864 | 1HB | GLU | A | 285 | 25.372 | 52.378 | −7.959 | 1.00 | 0.00 | H |
| ATOM | 1865 | 2HB | GLU | A | 285 | 26.262 | 51.948 | −6.479 | 1.00 | 0.00 | H |
| ATOM | 1866 | 1HG | GLU | A | 285 | 26.927 | 54.154 | −7.175 | 1.00 | 0.00 | H |
| ATOM | 1867 | 2HG | GLU | A | 285 | 28.291 | 53.045 | −7.452 | 1.00 | 0.00 | H |
| ATOM | 1868 | N | ALA | A | 286 | 28.000 | 49.850 | −6.298 | 1.00 | 0.00 | N |
| ATOM | 1869 | CA | ALA | A | 286 | 29.146 | 49.426 | −5.512 | 1.00 | 0.00 | C |
| ATOM | 1870 | C | ALA | A | 286 | 29.958 | 48.357 | −6.240 | 1.00 | 0.00 | C |
| ATOM | 1871 | O | ALA | A | 286 | 31.179 | 48.307 | −6.101 | 1.00 | 0.00 | O |
| ATOM | 1872 | CB | ALA | A | 286 | 28.691 | 48.907 | −4.146 | 1.00 | 0.00 | C |
| ATOM | 1873 | H | ALA | A | 286 | 27.093 | 49.811 | −5.854 | 1.00 | 0.00 | H |
| ATOM | 1874 | HA | ALA | A | 286 | 29.768 | 50.290 | −5.280 | 1.00 | 0.00 | H |
| ATOM | 1875 | 1HB | ALA | A | 286 | 29.560 | 48.592 | −3.568 | 1.00 | 0.00 | H |
| ATOM | 1876 | 2HB | ALA | A | 286 | 28.167 | 49.699 | −3.612 | 1.00 | 0.00 | H |
| ATOM | 1877 | 3HB | ALA | A | 286 | 28.021 | 48.058 | −4.285 | 1.00 | 0.00 | H |
| ATOM | 1878 | N | ARG | A | 287 | 29.284 | 47.487 | −6.992 | 1.00 | 0.00 | N |
| ATOM | 1879 | CA | ARG | A | 287 | 29.925 | 46.416 | −7.735 | 1.00 | 0.00 | C |
| ATOM | 1880 | C | ARG | A | 287 | 30.885 | 46.924 | −8.812 | 1.00 | 0.00 | C |
| ATOM | 1881 | O | ARG | A | 287 | 31.715 | 46.147 | −9.278 | 1.00 | 0.00 | O |
| ATOM | 1882 | CB | ARG | A | 287 | 28.863 | 45.507 | −8.352 | 1.00 | 0.00 | C |
| ATOM | 1883 | CG | ARG | A | 287 | 28.356 | 44.488 | −7.323 | 1.00 | 0.00 | C |
| ATOM | 1884 | CD | ARG | A | 287 | 27.022 | 43.862 | −7.742 | 1.00 | 0.00 | C |
| ATOM | 1885 | NE | ARG | A | 287 | 26.890 | 43.746 | −9.207 | 1.00 | 0.00 | N |
| ATOM | 1886 | CZ | ARG | A | 287 | 25.700 | 43.825 | −9.812 | 1.00 | 0.00 | C |
| ATOM | 1887 | NH1 | ARG | A | 287 | 24.626 | 43.448 | −9.131 | 1.00 | 0.00 | N1+ |
| ATOM | 1888 | NH2 | ARG | A | 287 | 25.609 | 44.283 | −11.055 | 1.00 | 0.00 | N |
| ATOM | 1889 | H | ARG | A | 287 | 28.279 | 47.555 | −7.065 | 1.00 | 0.00 | H |
| ATOM | 1890 | HA | ARG | A | 287 | 30.441 | 45.751 | −7.042 | 1.00 | 0.00 | H |
| ATOM | 1891 | 1HB | ARG | A | 287 | 28.021 | 46.110 | −8.693 | 1.00 | 0.00 | H |
| ATOM | 1892 | 2HB | ARG | A | 287 | 29.291 | 44.970 | −9.198 | 1.00 | 0.00 | H |
| ATOM | 1893 | 1HG | ARG | A | 287 | 29.087 | 43.687 | −7.214 | 1.00 | 0.00 | H |
| ATOM | 1894 | 2HG | ARG | A | 287 | 28.212 | 44.982 | −6.362 | 1.00 | 0.00 | H |
| ATOM | 1895 | 1HD | ARG | A | 287 | 26.940 | 42.861 | −7.317 | 1.00 | 0.00 | H |
| ATOM | 1896 | 2HD | ARG | A | 287 | 26.200 | 44.478 | −7.379 | 1.00 | 0.00 | H |
| ATOM | 1897 | HE | ARG | A | 287 | 27.726 | 43.604 | −9.754 | 1.00 | 0.00 | H |
| ATOM | 1898 | 1HH1 | ARG | A | 287 | 23.713 | 43.496 | −9.561 | 1.00 | 0.00 | H |
| ATOM | 1899 | 2HH1 | ARG | A | 287 | 24.721 | 43.113 | −8.183 | 1.00 | 0.00 | H |
| ATOM | 1900 | 1HH2 | ARG | A | 287 | 24.708 | 44.341 | −11.508 | 1.00 | 0.00 | H |
| ATOM | 1901 | 2HH2 | ARG | A | 287 | 26.442 | 44.573 | −11.548 | 1.00 | 0.00 | H |
| ATOM | 1902 | N | LYS | A | 288 | 30.781 | 48.184 | −9.234 | 1.00 | 0.00 | N |
| ATOM | 1903 | CA | LYS | A | 288 | 31.797 | 48.774 | −10.091 | 1.00 | 0.00 | C |
| ATOM | 1904 | C | LYS | A | 288 | 33.121 | 48.769 | −9.329 | 1.00 | 0.00 | C |
| ATOM | 1905 | O | LYS | A | 288 | 34.162 | 48.375 | −9.845 | 1.00 | 0.00 | O |
| ATOM | 1906 | CB | LYS | A | 288 | 31.424 | 50.221 | −10.439 | 1.00 | 0.00 | C |
| ATOM | 1907 | CG | LYS | A | 288 | 30.187 | 50.349 | −11.338 | 1.00 | 0.00 | C |
| ATOM | 1908 | CD | LYS | A | 288 | 29.610 | 51.763 | −11.182 | 1.00 | 0.00 | C |
| ATOM | 1909 | CE | LYS | A | 288 | 28.734 | 52.170 | −12.374 | 1.00 | 0.00 | C |
| ATOM | 1910 | NZ | LYS | A | 288 | 27.943 | 53.375 | −12.066 | 1.00 | 0.00 | N1+ |
| ATOM | 1911 | H | LYS | A | 288 | 29.988 | 48.745 | −8.959 | 1.00 | 0.00 | H |
| ATOM | 1912 | HA | LYS | A | 288 | 31.899 | 48.177 | −10.998 | 1.00 | 0.00 | H |
| ATOM | 1913 | 1HB | LYS | A | 288 | 31.213 | 50.773 | −9.523 | 1.00 | 0.00 | H |
| ATOM | 1914 | 2HB | LYS | A | 288 | 32.253 | 50.694 | −10.965 | 1.00 | 0.00 | H |
| ATOM | 1915 | 1HG | LYS | A | 288 | 30.472 | 50.177 | −12.376 | 1.00 | 0.00 | H |
| ATOM | 1916 | 2HG | LYS | A | 288 | 29.443 | 49.610 | −11.039 | 1.00 | 0.00 | H |
| ATOM | 1917 | 1HD | LYS | A | 288 | 28.997 | 51.809 | −10.282 | 1.00 | 0.00 | H |
| ATOM | 1918 | 2HD | LYS | A | 288 | 30.426 | 52.481 | −11.103 | 1.00 | 0.00 | H |
| ATOM | 1919 | 1HE | LYS | A | 288 | 29.367 | 52.380 | −13.236 | 1.00 | 0.00 | H |
| ATOM | 1920 | 2HE | LYS | A | 288 | 28.049 | 51.357 | −12.616 | 1.00 | 0.00 | H |
| ATOM | 1921 | 1HZ | LYS | A | 288 | 27.377 | 53.620 | −12.866 | 1.00 | 0.00 | H |
| ATOM | 1922 | 2HZ | LYS | A | 288 | 27.345 | 53.192 | −11.274 | 1.00 | 0.00 | H |
| ATOM | 1923 | 3HZ | LYS | A | 288 | 28.566 | 54.140 | −11.848 | 1.00 | 0.00 | H |
| ATOM | 1924 | N | PHE | A | 289 | 33.076 | 49.243 | −8.087 | 1.00 | 0.00 | N |
| ATOM | 1925 | CA | PHE | A | 289 | 34.248 | 49.704 | −7.368 | 1.00 | 0.00 | C |
| ATOM | 1926 | C | PHE | A | 289 | 34.636 | 48.717 | −6.272 | 1.00 | 0.00 | C |
| ATOM | 1927 | O | PHE | A | 289 | 35.463 | 49.028 | −5.418 | 1.00 | 0.00 | O |
| ATOM | 1928 | CB | PHE | A | 289 | 33.965 | 51.104 | −6.817 | 1.00 | 0.00 | C |
| ATOM | 1929 | CG | PHE | A | 289 | 33.732 | 52.146 | −7.893 | 1.00 | 0.00 | C |
| ATOM | 1930 | CD1 | PHE | A | 289 | 34.785 | 52.507 | −8.753 | 1.00 | 0.00 | C |
| ATOM | 1931 | CD2 | PHE | A | 289 | 32.475 | 52.760 | −8.058 | 1.00 | 0.00 | C |
| ATOM | 1932 | CE1 | PHE | A | 289 | 34.597 | 53.466 | −9.760 | 1.00 | 0.00 | C |
| ATOM | 1933 | CE2 | PHE | A | 289 | 32.280 | 53.722 | −9.065 | 1.00 | 0.00 | C |
| ATOM | 1934 | CZ | PHE | A | 289 | 33.343 | 54.078 | −9.913 | 1.00 | 0.00 | C |
| ATOM | 1935 | H | PHE | A | 289 | 32.193 | 49.296 | −7.598 | 1.00 | 0.00 | H |
| ATOM | 1936 | HA | PHE | A | 289 | 35.064 | 49.871 | −8.072 | 1.00 | 0.00 | H |
| ATOM | 1937 | 1HB | PHE | A | 289 | 33.071 | 51.076 | −6.194 | 1.00 | 0.00 | H |
| ATOM | 1938 | 2HB | PHE | A | 289 | 34.814 | 51.439 | −6.220 | 1.00 | 0.00 | H |
| ATOM | 1939 | HD1 | PHE | A | 289 | 35.755 | 52.044 | −8.644 | 1.00 | 0.00 | H |
| ATOM | 1940 | HD2 | PHE | A | 289 | 31.654 | 52.493 | −7.410 | 1.00 | 0.00 | H |
| ATOM | 1941 | HE1 | PHE | A | 289 | 35.415 | 53.731 | −10.414 | 1.00 | 0.00 | H |
| ATOM | 1942 | HE2 | PHE | A | 289 | 31.312 | 54.187 | −9.187 | 1.00 | 0.00 | H |
| ATOM | 1943 | HZ | PHE | A | 289 | 33.196 | 54.823 | −10.681 | 1.00 | 0.00 | H |
| ATOM | 1944 | N | SER | A | 290 | 34.052 | 47.521 | −6.303 | 1.00 | 0.00 | N |
| ATOM | 1945 | CA | SER | A | 290 | 34.524 | 46.369 | −5.552 | 1.00 | 0.00 | C |
| ATOM | 1946 | C | SER | A | 290 | 34.973 | 45.280 | −6.531 | 1.00 | 0.00 | C |
| ATOM | 1947 | O | SER | A | 290 | 35.242 | 44.144 | −6.141 | 1.00 | 0.00 | O |
| ATOM | 1948 | CB | SER | A | 290 | 33.426 | 45.887 | −4.595 | 1.00 | 0.00 | C |
| ATOM | 1949 | OG | SER | A | 290 | 32.132 | 45.976 | −5.162 | 1.00 | 0.00 | O |
| ATOM | 1950 | H | SER | A | 290 | 33.231 | 47.374 | −6.873 | 1.00 | 0.00 | H |
| ATOM | 1951 | HA | SER | A | 290 | 35.317 | 46.678 | −4.871 | 1.00 | 0.00 | H |
| ATOM | 1952 | 1HB | SER | A | 290 | 33.605 | 44.845 | −4.331 | 1.00 | 0.00 | H |
| ATOM | 1953 | 2HB | SER | A | 290 | 33.438 | 46.497 | −3.692 | 1.00 | 0.00 | H |
| ATOM | 1954 | HG | SER | A | 290 | 31.482 | 45.665 | −4.527 | 1.00 | 0.00 | H |
| ATOM | 1955 | N | TYR | A | 291 | 35.055 | 45.616 | −7.820 | 1.00 | 0.00 | N |
| ATOM | 1956 | CA | TYR | A | 291 | 35.599 | 44.690 | −8.784 | 1.00 | 0.00 | C |
| ATOM | 1957 | C | TYR | A | 291 | 37.116 | 44.707 | −8.598 | 1.00 | 0.00 | C |
| ATOM | 1958 | O | TYR | A | 291 | 37.768 | 45.699 | −8.927 | 1.00 | 0.00 | O |
| ATOM | 1959 | CB | TYR | A | 291 | 35.168 | 45.073 | −10.206 | 1.00 | 0.00 | C |
| ATOM | 1960 | CG | TYR | A | 291 | 35.644 | 44.108 | −11.278 | 1.00 | 0.00 | C |
| ATOM | 1961 | CD1 | TYR | A | 291 | 35.248 | 42.757 | −11.250 | 1.00 | 0.00 | C |
| ATOM | 1962 | CD2 | TYR | A | 291 | 36.486 | 44.539 | −12.320 | 1.00 | 0.00 | C |
| ATOM | 1963 | CE1 | TYR | A | 291 | 35.698 | 41.857 | −12.231 | 1.00 | 0.00 | C |
| ATOM | 1964 | CE2 | TYR | A | 291 | 36.930 | 43.643 | −13.306 | 1.00 | 0.00 | C |
| ATOM | 1965 | CZ | TYR | A | 291 | 36.543 | 42.291 | −13.266 | 1.00 | 0.00 | C |
| ATOM | 1966 | OH | TYR | A | 291 | 36.970 | 41.407 | −14.216 | 1.00 | 0.00 | O |
| ATOM | 1967 | H | TYR | A | 291 | 34.736 | 46.523 | −8.127 | 1.00 | 0.00 | H |
| ATOM | 1968 | HA | TYR | A | 291 | 35.154 | 43.706 | −8.636 | 1.00 | 0.00 | H |
| ATOM | 1969 | 1HB | TYR | A | 291 | 34.079 | 45.101 | −10.260 | 1.00 | 0.00 | H |
| ATOM | 1970 | 2HB | TYR | A | 291 | 35.570 | 46.055 | −10.456 | 1.00 | 0.00 | H |
| ATOM | 1971 | HD1 | TYR | A | 291 | 34.593 | 42.403 | −10.468 | 1.00 | 0.00 | H |
| ATOM | 1972 | HD2 | TYR | A | 291 | 36.801 | 45.571 | −12.369 | 1.00 | 0.00 | H |
| ATOM | 1973 | HE1 | TYR | A | 291 | 35.393 | 40.821 | −12.193 | 1.00 | 0.00 | H |
| ATOM | 1974 | HE2 | TYR | A | 291 | 37.571 | 43.990 | −14.103 | 1.00 | 0.00 | H |
| ATOM | 1975 | HH | TYR | A | 291 | 37.536 | 41.861 | −14.845 | 1.00 | 0.00 | H |
| ATOM | 1976 | N | ARG | A | 292 | 37.658 | 43.601 | −8.077 | 1.00 | 0.00 | N |
| ATOM | 1977 | CA | ARG | A | 292 | 39.075 | 43.308 | −7.892 | 1.00 | 0.00 | C |
| ATOM | 1978 | C | ARG | A | 292 | 39.546 | 43.710 | −6.486 | 1.00 | 0.00 | C |
| ATOM | 1979 | O | ARG | A | 292 | 40.662 | 44.200 | −6.338 | 1.00 | 0.00 | O |
| ATOM | 1980 | CB | ARG | A | 292 | 39.976 | 43.919 | −8.987 | 1.00 | 0.00 | C |
| ATOM | 1981 | CG | ARG | A | 292 | 39.522 | 43.636 | −10.435 | 1.00 | 0.00 | C |
| ATOM | 1982 | CD | ARG | A | 292 | 40.123 | 44.654 | −11.414 | 1.00 | 0.00 | C |
| ATOM | 1983 | NE | ARG | A | 292 | 39.767 | 46.042 | −11.053 | 1.00 | 0.00 | N |
| ATOM | 1984 | CZ | ARG | A | 292 | 40.600 | 47.083 | −11.222 | 1.00 | 0.00 | C |
| ATOM | 1985 | NH1 | ARG | A | 292 | 41.584 | 46.972 | −12.107 | 1.00 | 0.00 | N1+ |
| ATOM | 1986 | NH2 | ARG | A | 292 | 40.469 | 48.185 | −10.489 | 1.00 | 0.00 | N |
| ATOM | 1987 | H | ARG | A | 292 | 37.063 | 42.849 | −7.758 | 1.00 | 0.00 | H |
| ATOM | 1988 | HA | ARG | A | 292 | 39.266 | 42.263 | −8.137 | 1.00 | 0.00 | H |
| ATOM | 1989 | 1HB | ARG | A | 292 | 40.002 | 45.003 | −8.873 | 1.00 | 0.00 | H |
| ATOM | 1990 | 2HB | ARG | A | 292 | 40.986 | 43.520 | −8.892 | 1.00 | 0.00 | H |
| ATOM | 1991 | 1HG | ARG | A | 292 | 39.845 | 42.638 | −10.729 | 1.00 | 0.00 | H |
| ATOM | 1992 | 2HG | ARG | A | 292 | 38.435 | 43.697 | −10.494 | 1.00 | 0.00 | H |
| ATOM | 1993 | 1HD | ARG | A | 292 | 41.209 | 44.567 | −11.407 | 1.00 | 0.00 | H |
| ATOM | 1994 | 2HD | ARG | A | 292 | 39.750 | 44.456 | −12.419 | 1.00 | 0.00 | H |
| ATOM | 1995 | HE | ARG | A | 292 | 38.848 | 46.189 | −10.662 | 1.00 | 0.00 | H |
| ATOM | 1996 | 1HH1 | ARG | A | 292 | 42.222 | 47.741 | −12.252 | 1.00 | 0.00 | H |
| ATOM | 1997 | 2HH1 | ARG | A | 292 | 41.694 | 46.118 | −12.636 | 1.00 | 0.00 | H |
| ATOM | 1998 | 1HH2 | ARG | A | 292 | 41.101 | 48.960 | −10.626 | 1.00 | 0.00 | H |
| ATOM | 1999 | 2HH2 | ARG | A | 292 | 39.738 | 48.246 | −9.795 | 1.00 | 0.00 | H |
| ATOM | 2000 | N | SER | A | 293 | 38.755 | 43.475 | −5.434 | 1.00 | 0.00 | N |
| ATOM | 2001 | CA | SER | A | 293 | 39.187 | 43.837 | −4.091 | 1.00 | 0.00 | C |
| ATOM | 2002 | C | SER | A | 293 | 38.512 | 42.970 | −3.020 | 1.00 | 0.00 | C |
| ATOM | 2003 | O | SER | A | 293 | 37.691 | 42.108 | −3.330 | 1.00 | 0.00 | O |
| ATOM | 2004 | CB | SER | A | 293 | 38.928 | 45.332 | −3.870 | 1.00 | 0.00 | C |
| ATOM | 2005 | OG | SER | A | 293 | 37.542 | 45.609 | −3.784 | 1.00 | 0.00 | O |
| ATOM | 2006 | H | SER | A | 293 | 37.852 | 43.043 | −5.568 | 1.00 | 0.00 | H |
| ATOM | 2007 | HA | SER | A | 293 | 40.274 | 43.783 | −4.031 | 1.00 | 0.00 | H |
| ATOM | 2008 | 1HB | SER | A | 293 | 39.402 | 45.650 | −2.941 | 1.00 | 0.00 | H |
| ATOM | 2009 | 2HB | SER | A | 293 | 39.343 | 45.900 | −4.702 | 1.00 | 0.00 | H |
| ATOM | 2010 | HG | SER | A | 293 | 37.411 | 46.550 | −3.646 | 1.00 | 0.00 | H |
| ATOM | 2011 | N | ARG | A | 294 | 38.852 | 43.181 | −1.741 | 1.00 | 0.00 | N |
| ATOM | 2012 | CA | ARG | A | 294 | 38.394 | 42.338 | −0.641 | 1.00 | 0.00 | C |
| ATOM | 2013 | C | ARG | A | 294 | 36.948 | 42.658 | −0.229 | 1.00 | 0.00 | C |
| ATOM | 2014 | O | ARG | A | 294 | 36.636 | 42.720 | 0.961 | 1.00 | 0.00 | O |
| ATOM | 2015 | CB | ARG | A | 294 | 39.363 | 42.451 | 0.556 | 1.00 | 0.00 | C |
| ATOM | 2016 | CG | ARG | A | 294 | 39.528 | 43.875 | 1.128 | 1.00 | 0.00 | C |
| ATOM | 2017 | CD | ARG | A | 294 | 40.010 | 43.824 | 2.588 | 1.00 | 0.00 | C |
| ATOM | 2018 | NE | ARG | A | 294 | 40.237 | 45.172 | 3.144 | 1.00 | 0.00 | N |
| ATOM | 2019 | CZ | ARG | A | 294 | 40.343 | 45.502 | 4.442 | 1.00 | 0.00 | C |
| ATOM | 2020 | NH1 | ARG | A | 294 | 40.181 | 44.572 | 5.383 | 1.00 | 0.00 | N1+ |
| ATOM | 2021 | NH2 | ARG | A | 294 | 40.597 | 46.761 | 4.788 | 1.00 | 0.00 | N |
| ATOM | 2022 | H | ARG | A | 294 | 39.455 | 43.955 | −1.500 | 1.00 | 0.00 | H |
| ATOM | 2023 | HA | ARG | A | 294 | 38.519 | 41.289 | −0.909 | 1.00 | 0.00 | H |
| ATOM | 2024 | 1HB | ARG | A | 294 | 39.005 | 41.826 | 1.374 | 1.00 | 0.00 | H |
| ATOM | 2025 | 2HB | ARG | A | 294 | 40.356 | 42.119 | 0.253 | 1.00 | 0.00 | H |
| ATOM | 2026 | 1HG | ARG | A | 294 | 40.260 | 44.423 | 0.534 | 1.00 | 0.00 | H |
| ATOM | 2027 | 2HG | ARG | A | 294 | 38.570 | 44.395 | 1.092 | 1.00 | 0.00 | H |
| ATOM | 2028 | 1HD | ARG | A | 294 | 39.260 | 43.325 | 3.202 | 1.00 | 0.00 | H |
| ATOM | 2029 | 2HD | ARG | A | 294 | 40.949 | 43.271 | 2.642 | 1.00 | 0.00 | H |
| ATOM | 2030 | HE | ARG | A | 294 | 40.315 | 45.887 | 2.434 | 1.00 | 0.00 | H |
| ATOM | 2031 | 1HH1 | ARG | A | 294 | 40.261 | 44.822 | 6.359 | 1.00 | 0.00 | H |
| ATOM | 2032 | 2HH1 | ARG | A | 294 | 39.979 | 43.618 | 5.122 | 1.00 | 0.00 | H |
| ATOM | 2033 | 1HH2 | ARG | A | 294 | 40.677 | 47.010 | 5.763 | 1.00 | 0.00 | H |
| ATOM | 2034 | 2HH2 | ARG | A | 294 | 40.711 | 47.467 | 4.075 | 1.00 | 0.00 | H |
| ATOM | 2035 | N | VAL | A | 295 | 36.046 | 42.852 | −1.188 | 1.00 | 0.00 | N |
| ATOM | 2036 | CA | VAL | A | 295 | 34.715 | 43.379 | −0.946 | 1.00 | 0.00 | C |
| ATOM | 2037 | C | VAL | A | 295 | 33.719 | 42.549 | −1.752 | 1.00 | 0.00 | C |
| ATOM | 2038 | O | VAL | A | 295 | 33.739 | 42.568 | −2.976 | 1.00 | 0.00 | O |
| ATOM | 2039 | CB | VAL | A | 295 | 34.663 | 44.867 | −1.339 | 1.00 | 0.00 | C |
| ATOM | 2040 | CG1 | VAL | A | 295 | 33.277 | 45.455 | −1.033 | 1.00 | 0.00 | C |
| ATOM | 2041 | CG2 | VAL | A | 295 | 35.716 | 45.682 | −0.581 | 1.00 | 0.00 | C |
| ATOM | 2042 | H | VAL | A | 295 | 36.272 | 42.628 | −2.147 | 1.00 | 0.00 | H |
| ATOM | 2043 | HA | VAL | A | 295 | 34.499 | 43.346 | 0.122 | 1.00 | 0.00 | H |
| ATOM | 2044 | HB | VAL | A | 295 | 34.875 | 44.970 | −2.403 | 1.00 | 0.00 | H |
| ATOM | 2045 | 1HG1 | VAL | A | 295 | 33.258 | 46.507 | −1.316 | 1.00 | 0.00 | H |
| ATOM | 2046 | 2HG1 | VAL | A | 295 | 32.519 | 44.913 | −1.598 | 1.00 | 0.00 | H |
| ATOM | 2047 | 3HG1 | VAL | A | 295 | 33.070 | 45.363 | 0.034 | 1.00 | 0.00 | H |
| ATOM | 2048 | 1HG2 | VAL | A | 295 | 35.652 | 46.728 | −0.881 | 1.00 | 0.00 | H |
| ATOM | 2049 | 2HG2 | VAL | A | 295 | 35.536 | 45.601 | 0.491 | 1.00 | 0.00 | H |
| ATOM | 2050 | 3HG2 | VAL | A | 295 | 36.709 | 45.298 | −0.812 | 1.00 | 0.00 | H |
| ATOM | 2051 | N | ARG | A | 296 | 32.823 | 41.844 | −1.063 | 1.00 | 0.00 | N |
| ATOM | 2052 | CA | ARG | A | 296 | 31.770 | 41.061 | −1.682 | 1.00 | 0.00 | C |
| ATOM | 2053 | C | ARG | A | 296 | 30.447 | 41.647 | −1.182 | 1.00 | 0.00 | C |
| ATOM | 2054 | O | ARG | A | 296 | 30.099 | 41.440 | −0.016 | 1.00 | 0.00 | O |
| ATOM | 2055 | CB | ARG | A | 296 | 31.966 | 39.590 | −1.290 | 1.00 | 0.00 | C |
| ATOM | 2056 | CG | ARG | A | 296 | 30.994 | 38.616 | −1.977 | 1.00 | 0.00 | C |
| ATOM | 2057 | CD | ARG | A | 296 | 29.710 | 38.347 | −1.173 | 1.00 | 0.00 | C |
| ATOM | 2058 | NE | ARG | A | 296 | 28.509 | 38.973 | −1.761 | 1.00 | 0.00 | N |
| ATOM | 2059 | CZ | ARG | A | 296 | 27.307 | 38.380 | −1.874 | 1.00 | 0.00 | C |
| ATOM | 2060 | NH1 | ARG | A | 296 | 27.047 | 37.241 | −1.241 | 1.00 | 0.00 | N1+ |
| ATOM | 2061 | NH2 | ARG | A | 296 | 26.381 | 38.943 | −2.637 | 1.00 | 0.00 | N |
| ATOM | 2062 | H | ARG | A | 296 | 32.854 | 41.836 | −0.053 | 1.00 | 0.00 | H |
| ATOM | 2063 | HA | ARG | A | 296 | 31.869 | 41.112 | −2.767 | 1.00 | 0.00 | H |
| ATOM | 2064 | 1HB | ARG | A | 296 | 32.974 | 39.274 | −1.556 | 1.00 | 0.00 | H |
| ATOM | 2065 | 2HB | ARG | A | 296 | 31.822 | 39.478 | −0.216 | 1.00 | 0.00 | H |
| ATOM | 2066 | 1HG | ARG | A | 296 | 30.691 | 39.023 | −2.942 | 1.00 | 0.00 | H |
| ATOM | 2067 | 2HG | ARG | A | 296 | 31.487 | 37.655 | −2.127 | 1.00 | 0.00 | H |
| ATOM | 2068 | 1HD | ARG | A | 296 | 29.529 | 37.273 | −1.125 | 1.00 | 0.00 | H |
| ATOM | 2069 | 2HD | ARG | A | 296 | 29.823 | 38.742 | −0.164 | 1.00 | 0.00 | H |
| ATOM | 2070 | HE | ARG | A | 296 | 28.639 | 39.918 | −2.092 | 1.00 | 0.00 | H |
| ATOM | 2071 | 1HH1 | ARG | A | 296 | 26.140 | 36.807 | −1.335 | 1.00 | 0.00 | H |
| ATOM | 2072 | 2HH1 | ARG | A | 296 | 27.756 | 36.810 | −0.665 | 1.00 | 0.00 | H |
| ATOM | 2073 | 1HH2 | ARG | A | 296 | 25.473 | 38.510 | −2.732 | 1.00 | 0.00 | H |
| ATOM | 2074 | 2HH2 | ARG | A | 296 | 26.582 | 39.805 | −3.124 | 1.00 | 0.00 | H |
| ATOM | 2075 | N | PRO | A | 297 | 29.714 | 42.390 | −2.026 | 1.00 | 0.00 | N |
| ATOM | 2076 | CA | PRO | A | 297 | 28.584 | 43.196 | −1.573 | 1.00 | 0.00 | C |
| ATOM | 2077 | C | PRO | A | 297 | 27.278 | 42.423 | −1.798 | 1.00 | 0.00 | C |
| ATOM | 2078 | O | PRO | A | 297 | 27.277 | 41.414 | −2.507 | 1.00 | 0.00 | O |
| ATOM | 2079 | CB | PRO | A | 297 | 28.634 | 44.468 | −2.421 | 1.00 | 0.00 | C |
| ATOM | 2080 | CG | PRO | A | 297 | 29.207 | 43.973 | −3.746 | 1.00 | 0.00 | C |
| ATOM | 2081 | CD | PRO | A | 297 | 30.214 | 42.917 | −3.294 | 1.00 | 0.00 | C |
| ATOM | 2082 | HA | PRO | A | 297 | 28.695 | 43.414 | −0.511 | 1.00 | 0.00 | H |
| ATOM | 2083 | 1HB | PRO | A | 297 | 27.629 | 44.875 | −2.526 | 1.00 | 0.00 | H |
| ATOM | 2084 | 2HB | PRO | A | 297 | 29.275 | 45.204 | −1.936 | 1.00 | 0.00 | H |
| ATOM | 2085 | 1HG | PRO | A | 297 | 28.405 | 43.559 | −4.357 | 1.00 | 0.00 | H |
| ATOM | 2086 | 2HG | PRO | A | 297 | 29.673 | 44.804 | −4.274 | 1.00 | 0.00 | H |
| ATOM | 2087 | 1HD | PRO | A | 297 | 31.200 | 43.356 | −3.141 | 1.00 | 0.00 | H |
| ATOM | 2088 | 2HD | PRO | A | 297 | 30.286 | 42.108 | −4.021 | 1.00 | 0.00 | H |
| ATOM | 2089 | N | CYS | A | 298 | 26.169 | 42.871 | −1.210 | 1.00 | 0.00 | N |
| ATOM | 2090 | CA | CYS | A | 298 | 24.891 | 42.167 | −1.192 | 1.00 | 0.00 | C |
| ATOM | 2091 | C | CYS | A | 298 | 23.780 | 43.197 | −0.971 | 1.00 | 0.00 | C |
| ATOM | 2092 | O | CYS | A | 298 | 24.037 | 44.242 | −0.374 | 1.00 | 0.00 | O |
| ATOM | 2093 | CB | CYS | A | 298 | 24.924 | 41.140 | −0.049 | 1.00 | 0.00 | C |
| ATOM | 2094 | SG | CYS | A | 298 | 23.385 | 40.185 | 0.073 | 1.00 | 0.00 | S |
| ATOM | 2095 | H | CYS | A | 298 | 26.176 | 43.761 | −0.733 | 1.00 | 0.00 | H |
| ATOM | 2096 | HA | CYS | A | 298 | 24.746 | 41.651 | −2.142 | 1.00 | 0.00 | H |
| ATOM | 2097 | 1HB | CYS | A | 298 | 25.741 | 40.439 | −0.213 | 1.00 | 0.00 | H |
| ATOM | 2098 | 2HB | CYS | A | 298 | 25.073 | 41.657 | 0.899 | 1.00 | 0.00 | H |
| ATOM | 2099 | HG | CYS | A | 298 | 23.463 | 39.468 | 0.904 | 1.00 | 0.00 | H |
| ATOM | 2100 | N | VAL | A | 299 | 22.560 | 42.899 | −1.422 | 1.00 | 0.00 | N |
| ATOM | 2101 | CA | VAL | A | 299 | 21.386 | 43.734 | −1.217 | 1.00 | 0.00 | C |
| ATOM | 2102 | C | VAL | A | 299 | 20.258 | 42.838 | −0.706 | 1.00 | 0.00 | C |
| ATOM | 2103 | O | VAL | A | 299 | 20.076 | 41.732 | −1.219 | 1.00 | 0.00 | O |
| ATOM | 2104 | CB | VAL | A | 299 | 21.004 | 44.459 | −2.521 | 1.00 | 0.00 | C |
| ATOM | 2105 | CG1 | VAL | A | 299 | 20.693 | 43.513 | −3.689 | 1.00 | 0.00 | C |
| ATOM | 2106 | CG2 | VAL | A | 299 | 19.814 | 45.400 | −2.300 | 1.00 | 0.00 | C |
| ATOM | 2107 | H | VAL | A | 299 | 22.408 | 42.047 | −1.942 | 1.00 | 0.00 | H |
| ATOM | 2108 | HA | VAL | A | 299 | 21.629 | 44.539 | −0.524 | 1.00 | 0.00 | H |
| ATOM | 2109 | HB | VAL | A | 299 | 21.797 | 45.153 | −2.797 | 1.00 | 0.00 | H |
| ATOM | 2110 | 1HG1 | VAL | A | 299 | 20.432 | 44.098 | −4.571 | 1.00 | 0.00 | H |
| ATOM | 2111 | 2HG1 | VAL | A | 299 | 21.569 | 42.901 | −3.905 | 1.00 | 0.00 | H |
| ATOM | 2112 | 3HG1 | VAL | A | 299 | 19.857 | 42.867 | −3.422 | 1.00 | 0.00 | H |
| ATOM | 2113 | 1HG2 | VAL | A | 299 | 19.566 | 45.898 | −3.237 | 1.00 | 0.00 | H |
| ATOM | 2114 | 2HG2 | VAL | A | 299 | 18.955 | 44.825 | −1.956 | 1.00 | 0.00 | H |
| ATOM | 2115 | 3HG2 | VAL | A | 299 | 20.076 | 46.147 | −1.550 | 1.00 | 0.00 | H |
| ATOM | 2116 | N | VAL | A | 300 | 19.497 | 43.303 | 0.284 | 1.00 | 0.00 | N |
| ATOM | 2117 | CA | VAL | A | 300 | 18.382 | 42.565 | 0.860 | 1.00 | 0.00 | C |
| ATOM | 2118 | C | VAL | A | 300 | 17.181 | 43.503 | 0.983 | 1.00 | 0.00 | C |
| ATOM | 2119 | O | VAL | A | 300 | 17.339 | 44.702 | 1.194 | 1.00 | 0.00 | O |
| ATOM | 2120 | CB | VAL | A | 300 | 18.782 | 41.913 | 2.195 | 1.00 | 0.00 | C |
| ATOM | 2121 | CG1 | VAL | A | 300 | 19.962 | 40.955 | 1.985 | 1.00 | 0.00 | C |
| ATOM | 2122 | CG2 | VAL | A | 300 | 19.134 | 42.937 | 3.280 | 1.00 | 0.00 | C |
| ATOM | 2123 | H | VAL | A | 300 | 19.682 | 44.214 | 0.677 | 1.00 | 0.00 | H |
| ATOM | 2124 | HA | VAL | A | 300 | 18.190 | 41.673 | 0.265 | 1.00 | 0.00 | H |
| ATOM | 2125 | HB | VAL | A | 300 | 17.920 | 41.401 | 2.622 | 1.00 | 0.00 | H |
| ATOM | 2126 | 1HG1 | VAL | A | 300 | 20.235 | 40.500 | 2.937 | 1.00 | 0.00 | H |
| ATOM | 2127 | 2HG1 | VAL | A | 300 | 19.677 | 40.175 | 1.279 | 1.00 | 0.00 | H |
| ATOM | 2128 | 3HG1 | VAL | A | 300 | 20.814 | 41.508 | 1.590 | 1.00 | 0.00 | H |
| ATOM | 2129 | 1HG2 | VAL | A | 300 | 19.408 | 42.416 | 4.197 | 1.00 | 0.00 | H |
| ATOM | 2130 | 2HG2 | VAL | A | 300 | 19.972 | 43.548 | 2.946 | 1.00 | 0.00 | H |
| ATOM | 2131 | 3HG2 | VAL | A | 300 | 18.272 | 43.576 | 3.470 | 1.00 | 0.00 | H |
| ATOM | 2132 | N | TYR | A | 301 | 15.978 | 42.975 | 0.777 | 1.00 | 0.00 | N |
| ATOM | 2133 | CA | TYR | A | 301 | 14.814 | 43.778 | 0.444 | 1.00 | 0.00 | C |
| ATOM | 2134 | C | TYR | A | 301 | 13.573 | 43.079 | 0.998 | 1.00 | 0.00 | C |
| ATOM | 2135 | O | TYR | A | 301 | 13.425 | 41.864 | 0.846 | 1.00 | 0.00 | O |
| ATOM | 2136 | CB | TYR | A | 301 | 14.739 | 43.992 | −1.081 | 1.00 | 0.00 | C |
| ATOM | 2137 | CG | TYR | A | 301 | 15.182 | 42.820 | −1.945 | 1.00 | 0.00 | C |
| ATOM | 2138 | CD1 | TYR | A | 301 | 14.244 | 41.944 | −2.521 | 1.00 | 0.00 | C |
| ATOM | 2139 | CD2 | TYR | A | 301 | 16.550 | 42.590 | −2.186 | 1.00 | 0.00 | C |
| ATOM | 2140 | CE1 | TYR | A | 301 | 14.667 | 40.859 | −3.310 | 1.00 | 0.00 | C |
| ATOM | 2141 | CE2 | TYR | A | 301 | 16.978 | 41.484 | −2.936 | 1.00 | 0.00 | C |
| ATOM | 2142 | CZ | TYR | A | 301 | 16.038 | 40.606 | −3.502 | 1.00 | 0.00 | C |
| ATOM | 2143 | OH | TYR | A | 301 | 16.443 | 39.522 | −4.226 | 1.00 | 0.00 | O |
| ATOM | 2144 | H | TYR | A | 301 | 15.846 | 41.977 | 0.850 | 1.00 | 0.00 | H |
| ATOM | 2145 | HA | TYR | A | 301 | 14.942 | 44.786 | 0.837 | 1.00 | 0.00 | H |
| ATOM | 2146 | 1HB | TYR | A | 301 | 13.710 | 44.206 | −1.368 | 1.00 | 0.00 | H |
| ATOM | 2147 | 2HB | TYR | A | 301 | 15.377 | 44.830 | −1.362 | 1.00 | 0.00 | H |
| ATOM | 2148 | HD1 | TYR | A | 301 | 13.188 | 42.100 | −2.359 | 1.00 | 0.00 | H |
| ATOM | 2149 | HD2 | TYR | A | 301 | 17.290 | 43.271 | −1.790 | 1.00 | 0.00 | H |
| ATOM | 2150 | HE1 | TYR | A | 301 | 13.937 | 40.212 | −3.774 | 1.00 | 0.00 | H |
| ATOM | 2151 | HE2 | TYR | A | 301 | 18.034 | 41.305 | −3.081 | 1.00 | 0.00 | H |
| ATOM | 2152 | HH | TYR | A | 301 | 17.402 | 39.499 | −4.259 | 1.00 | 0.00 | H |
| ATOM | 2153 | N | GLY | A | 302 | 12.722 | 43.836 | 1.690 | 1.00 | 0.00 | N |
| ATOM | 2154 | CA | GLY | A | 302 | 11.485 | 43.345 | 2.273 | 1.00 | 0.00 | C |
| ATOM | 2155 | C | GLY | A | 302 | 10.548 | 42.731 | 1.230 | 1.00 | 0.00 | C |
| ATOM | 2156 | O | GLY | A | 302 | 10.742 | 42.893 | 0.026 | 1.00 | 0.00 | O |
| ATOM | 2157 | H | GLY | A | 302 | 12.921 | 44.815 | 1.836 | 1.00 | 0.00 | H |
| ATOM | 2158 | 1HA | GLY | A | 302 | 11.711 | 42.578 | 3.014 | 1.00 | 0.00 | H |
| ATOM | 2159 | 2HA | GLY | A | 302 | 10.957 | 44.170 | 2.754 | 1.00 | 0.00 | H |
| ATOM | 2160 | N | GLY | A | 303 | 9.537 | 41.994 | 1.689 | 1.00 | 0.00 | N |
| ATOM | 2161 | CA | GLY | A | 303 | 8.539 | 41.394 | 0.817 | 1.00 | 0.00 | C |
| ATOM | 2162 | C | GLY | A | 303 | 9.040 | 40.050 | 0.292 | 1.00 | 0.00 | C |
| ATOM | 2163 | O | GLY | A | 303 | 8.352 | 39.039 | 0.387 | 1.00 | 0.00 | O |
| ATOM | 2164 | H | GLY | A | 303 | 9.441 | 41.831 | 2.682 | 1.00 | 0.00 | H |
| ATOM | 2165 | 1HA | GLY | A | 303 | 7.615 | 41.238 | 1.374 | 1.00 | 0.00 | H |
| ATOM | 2166 | 2HA | GLY | A | 303 | 8.347 | 42.057 | −0.026 | 1.00 | 0.00 | H |
| ATOM | 2167 | N | ALA | A | 304 | 10.250 | 40.038 | −0.266 | 1.00 | 0.00 | N |
| ATOM | 2168 | CA | ALA | A | 304 | 10.794 | 38.859 | −0.914 | 1.00 | 0.00 | C |
| ATOM | 2169 | C | ALA | A | 304 | 11.077 | 37.750 | 0.101 | 1.00 | 0.00 | C |
| ATOM | 2170 | O | ALA | A | 304 | 11.351 | 38.028 | 1.273 | 1.00 | 0.00 | O |
| ATOM | 2171 | CB | ALA | A | 304 | 12.075 | 39.240 | −1.656 | 1.00 | 0.00 | C |
| ATOM | 2172 | H | ALA | A | 304 | 10.823 | 40.869 | −0.247 | 1.00 | 0.00 | H |
| ATOM | 2173 | HA | ALA | A | 304 | 10.095 | 38.506 | −1.672 | 1.00 | 0.00 | H |
| ATOM | 2174 | 1HB | ALA | A | 304 | 12.488 | 38.357 | −2.145 | 1.00 | 0.00 | H |
| ATOM | 2175 | 2HB | ALA | A | 304 | 11.851 | 39.998 | −2.405 | 1.00 | 0.00 | H |
| ATOM | 2176 | 3HB | ALA | A | 304 | 12.802 | 39.635 | −0.946 | 1.00 | 0.00 | H |
| ATOM | 2177 | N | ASP | A | 305 | 11.078 | 36.502 | −0.382 | 1.00 | 0.00 | N |
| ATOM | 2178 | CA | ASP | A | 305 | 11.501 | 35.325 | 0.367 | 1.00 | 0.00 | C |
| ATOM | 2179 | C | ASP | A | 305 | 12.806 | 35.634 | 1.105 | 1.00 | 0.00 | C |
| ATOM | 2180 | O | ASP | A | 305 | 13.834 | 35.883 | 0.472 | 1.00 | 0.00 | O |
| ATOM | 2181 | CB | ASP | A | 305 | 11.704 | 34.142 | −0.597 | 1.00 | 0.00 | C |
| ATOM | 2182 | CG | ASP | A | 305 | 12.380 | 32.954 | 0.073 | 1.00 | 0.00 | C |
| ATOM | 2183 | OD1 | ASP | A | 305 | 12.213 | 32.800 | 1.298 | 1.00 | 0.00 | O |
| ATOM | 2184 | OD2 | ASP | A | 305 | 13.125 | 32.230 | −0.628 | 1.00 | 0.00 | O1− |
| ATOM | 2185 | H | ASP | A | 305 | 10.771 | 36.326 | −1.327 | 1.00 | 0.00 | H |
| ATOM | 2186 | HA | ASP | A | 305 | 10.723 | 35.046 | 1.077 | 1.00 | 0.00 | H |
| ATOM | 2187 | 1HB | ASP | A | 305 | 10.737 | 33.810 | −0.974 | 1.00 | 0.00 | H |
| ATOM | 2188 | 2HB | ASP | A | 305 | 12.331 | 34.457 | −1.432 | 1.00 | 0.00 | H |
| ATOM | 2189 | N | ILE | A | 306 | 12.773 | 35.601 | 2.438 | 1.00 | 0.00 | N |
| ATOM | 2190 | CA | ILE | A | 306 | 13.958 | 35.726 | 3.270 | 1.00 | 0.00 | C |
| ATOM | 2191 | C | ILE | A | 306 | 15.004 | 34.708 | 2.805 | 1.00 | 0.00 | C |
| ATOM | 2192 | O | ILE | A | 306 | 16.204 | 34.982 | 2.787 | 1.00 | 0.00 | O |
| ATOM | 2193 | CB | ILE | A | 306 | 13.570 | 35.547 | 4.754 | 1.00 | 0.00 | C |
| ATOM | 2194 | CG1 | ILE | A | 306 | 14.736 | 35.965 | 5.665 | 1.00 | 0.00 | C |
| ATOM | 2195 | CG2 | ILE | A | 306 | 13.081 | 34.123 | 5.069 | 1.00 | 0.00 | C |
| ATOM | 2196 | CD1 | ILE | A | 306 | 14.466 | 35.723 | 7.151 | 1.00 | 0.00 | C |
| ATOM | 2197 | H | ILE | A | 306 | 11.893 | 35.484 | 2.918 | 1.00 | 0.00 | H |
| ATOM | 2198 | HA | ILE | A | 306 | 14.323 | 36.752 | 3.231 | 1.00 | 0.00 | H |
| ATOM | 2199 | HB | ILE | A | 306 | 12.644 | 36.086 | 4.956 | 1.00 | 0.00 | H |
| ATOM | 2200 | 1HG1 | ILE | A | 306 | 15.627 | 35.398 | 5.398 | 1.00 | 0.00 | H |
| ATOM | 2201 | 2HG1 | ILE | A | 306 | 14.932 | 37.030 | 5.538 | 1.00 | 0.00 | H |
| ATOM | 2202 | 1HG2 | ILE | A | 306 | 12.821 | 34.053 | 6.125 | 1.00 | 0.00 | H |
| ATOM | 2203 | 2HG2 | ILE | A | 306 | 12.202 | 33.898 | 4.464 | 1.00 | 0.00 | H |
| ATOM | 2204 | 3HG2 | ILE | A | 306 | 13.871 | 33.408 | 4.841 | 1.00 | 0.00 | H |
| ATOM | 2205 | 1HD1 | ILE | A | 306 | 15.330 | 36.041 | 7.735 | 1.00 | 0.00 | H |
| ATOM | 2206 | 2HD1 | ILE | A | 306 | 13.590 | 36.294 | 7.460 | 1.00 | 0.00 | H |
| ATOM | 2207 | 3HD1 | ILE | A | 306 | 14.285 | 34.662 | 7.320 | 1.00 | 0.00 | H |
| ATOM | 2208 | N | GLY | A | 307 | 14.533 | 33.529 | 2.414 | 1.00 | 0.00 | N |
| ATOM | 2209 | CA | GLY | A | 307 | 15.319 | 32.462 | 1.860 | 1.00 | 0.00 | C |
| ATOM | 2210 | C | GLY | A | 307 | 16.163 | 32.949 | 0.686 | 1.00 | 0.00 | C |
| ATOM | 2211 | O | GLY | A | 307 | 17.314 | 32.550 | 0.568 | 1.00 | 0.00 | O |
| ATOM | 2212 | H | GLY | A | 307 | 13.547 | 33.326 | 2.495 | 1.00 | 0.00 | H |
| ATOM | 2213 | 1HA | GLY | A | 307 | 15.986 | 32.066 | 2.626 | 1.00 | 0.00 | H |
| ATOM | 2214 | 2HA | GLY | A | 307 | 14.659 | 31.669 | 1.509 | 1.00 | 0.00 | H |
| ATOM | 2215 | N | GLN | A | 308 | 15.624 | 33.783 | −0.204 | 1.00 | 0.00 | N |
| ATOM | 2216 | CA | GLN | A | 308 | 16.333 | 34.243 | −1.391 | 1.00 | 0.00 | C |
| ATOM | 2217 | C | GLN | A | 308 | 17.581 | 35.024 | −0.997 | 1.00 | 0.00 | C |
| ATOM | 2218 | O | GLN | A | 308 | 18.546 | 35.083 | −1.756 | 1.00 | 0.00 | O |
| ATOM | 2219 | CB | GLN | A | 308 | 15.381 | 35.073 | −2.272 | 1.00 | 0.00 | C |
| ATOM | 2220 | CG | GLN | A | 308 | 15.941 | 35.518 | −3.633 | 1.00 | 0.00 | C |
| ATOM | 2221 | CD | GLN | A | 308 | 16.122 | 34.381 | −4.635 | 1.00 | 0.00 | C |
| ATOM | 2222 | NE2 | GLN | A | 308 | 16.133 | 34.700 | −5.921 | 1.00 | 0.00 | N |
| ATOM | 2223 | OE1 | GLN | A | 308 | 16.234 | 33.211 | −4.272 | 1.00 | 0.00 | O |
| ATOM | 2224 | H | GLN | A | 308 | 14.683 | 34.125 | −0.070 | 1.00 | 0.00 | H |
| ATOM | 2225 | HA | GLN | A | 308 | 16.565 | 33.390 | −2.030 | 1.00 | 0.00 | H |
| ATOM | 2226 | 1HB | GLN | A | 308 | 14.485 | 34.492 | −2.488 | 1.00 | 0.00 | H |
| ATOM | 2227 | 2HB | GLN | A | 308 | 15.103 | 35.986 | −1.746 | 1.00 | 0.00 | H |
| ATOM | 2228 | 1HG | GLN | A | 308 | 15.262 | 36.240 | −4.087 | 1.00 | 0.00 | H |
| ATOM | 2229 | 2HG | GLN | A | 308 | 16.919 | 35.979 | −3.492 | 1.00 | 0.00 | H |
| ATOM | 2230 | 1HE2 | GLN | A | 308 | 16.250 | 33.979 | −6.619 | 1.00 | 0.00 | H |
| ATOM | 2231 | 2HE2 | GLN | A | 308 | 16.025 | 35.663 | −6.203 | 1.00 | 0.00 | H |
| ATOM | 2232 | N | GLN | A | 309 | 17.545 | 35.625 | 0.185 | 1.00 | 0.00 | N |
| ATOM | 2233 | CA | GLN | A | 309 | 18.573 | 36.518 | 0.666 | 1.00 | 0.00 | C |
| ATOM | 2234 | C | GLN | A | 309 | 19.501 | 35.729 | 1.595 | 1.00 | 0.00 | C |
| ATOM | 2235 | O | GLN | A | 309 | 20.710 | 35.945 | 1.589 | 1.00 | 0.00 | O |
| ATOM | 2236 | CB | GLN | A | 309 | 17.870 | 37.714 | 1.312 | 1.00 | 0.00 | C |
| ATOM | 2237 | CG | GLN | A | 309 | 17.142 | 38.529 | 0.224 | 1.00 | 0.00 | C |
| ATOM | 2238 | CD | GLN | A | 309 | 16.001 | 39.382 | 0.761 | 1.00 | 0.00 | C |
| ATOM | 2239 | NE2 | GLN | A | 309 | 15.404 | 40.198 | −0.091 | 1.00 | 0.00 | N |
| ATOM | 2240 | OE1 | GLN | A | 309 | 15.640 | 39.322 | 1.929 | 1.00 | 0.00 | O |
| ATOM | 2241 | H | GLN | A | 309 | 16.766 | 35.469 | 0.808 | 1.00 | 0.00 | H |
| ATOM | 2242 | HA | GLN | A | 309 | 19.082 | 36.977 | −0.182 | 1.00 | 0.00 | H |
| ATOM | 2243 | 1HB | GLN | A | 309 | 17.146 | 37.357 | 2.045 | 1.00 | 0.00 | H |
| ATOM | 2244 | 2HB | GLN | A | 309 | 18.607 | 38.345 | 1.807 | 1.00 | 0.00 | H |
| ATOM | 2245 | 1HG | GLN | A | 309 | 17.849 | 39.201 | −0.261 | 1.00 | 0.00 | H |
| ATOM | 2246 | 2HG | GLN | A | 309 | 16.719 | 37.851 | −0.517 | 1.00 | 0.00 | H |
| ATOM | 2247 | 1HE2 | GLN | A | 309 | 14.642 | 40.783 | 0.221 | 1.00 | 0.00 | H |
| ATOM | 2248 | 2HE2 | GLN | A | 309 | 15.709 | 40.236 | −1.053 | 1.00 | 0.00 | H |
| ATOM | 2249 | N | ILE | A | 310 | 18.967 | 34.758 | 2.342 | 1.00 | 0.00 | N |
| ATOM | 2250 | CA | ILE | A | 310 | 19.774 | 33.761 | 3.035 | 1.00 | 0.00 | C |
| ATOM | 2251 | C | ILE | A | 310 | 20.632 | 33.034 | 1.995 | 1.00 | 0.00 | C |
| ATOM | 2252 | O | ILE | A | 310 | 21.827 | 32.826 | 2.204 | 1.00 | 0.00 | O |
| ATOM | 2253 | CB | ILE | A | 310 | 18.872 | 32.792 | 3.830 | 1.00 | 0.00 | C |
| ATOM | 2254 | CG1 | ILE | A | 310 | 18.234 | 33.536 | 5.017 | 1.00 | 0.00 | C |
| ATOM | 2255 | CG2 | ILE | A | 310 | 19.644 | 31.565 | 4.344 | 1.00 | 0.00 | C |
| ATOM | 2256 | CD1 | ILE | A | 310 | 17.119 | 32.750 | 5.714 | 1.00 | 0.00 | C |
| ATOM | 2257 | H | ILE | A | 310 | 17.964 | 34.694 | 2.445 | 1.00 | 0.00 | H |
| ATOM | 2258 | HA | ILE | A | 310 | 20.369 | 34.247 | 3.808 | 1.00 | 0.00 | H |
| ATOM | 2259 | HB | ILE | A | 310 | 18.109 | 32.380 | 3.169 | 1.00 | 0.00 | H |
| ATOM | 2260 | 1HG1 | ILE | A | 310 | 18.997 | 33.748 | 5.766 | 1.00 | 0.00 | H |
| ATOM | 2261 | 2HG1 | ILE | A | 310 | 17.799 | 34.473 | 4.668 | 1.00 | 0.00 | H |
| ATOM | 2262 | 1HG2 | ILE | A | 310 | 18.966 | 30.914 | 4.897 | 1.00 | 0.00 | H |
| ATOM | 2263 | 2HG2 | ILE | A | 310 | 20.064 | 31.019 | 3.500 | 1.00 | 0.00 | H |
| ATOM | 2264 | 3HG2 | ILE | A | 310 | 20.450 | 31.891 | 5.002 | 1.00 | 0.00 | H |
| ATOM | 2265 | 1HD1 | ILE | A | 310 | 16.719 | 33.340 | 6.539 | 1.00 | 0.00 | H |
| ATOM | 2266 | 2HD1 | ILE | A | 310 | 16.323 | 32.538 | 5.001 | 1.00 | 0.00 | H |
| ATOM | 2267 | 3HD1 | ILE | A | 310 | 17.521 | 31.813 | 6.099 | 1.00 | 0.00 | H |
| ATOM | 2268 | N | ARG | A | 311 | 20.030 | 32.681 | 0.857 | 1.00 | 0.00 | N |
| ATOM | 2269 | CA | ARG | A | 311 | 20.674 | 31.976 | −0.241 | 1.00 | 0.00 | C |
| ATOM | 2270 | C | ARG | A | 311 | 21.741 | 32.839 | −0.940 | 1.00 | 0.00 | C |
| ATOM | 2271 | O | ARG | A | 311 | 22.281 | 32.404 | −1.963 | 1.00 | 0.00 | O |
| ATOM | 2272 | CB | ARG | A | 311 | 19.617 | 31.454 | −1.245 | 1.00 | 0.00 | C |
| ATOM | 2273 | CG | ARG | A | 311 | 18.820 | 30.240 | −0.725 | 1.00 | 0.00 | C |
| ATOM | 2274 | CD | ARG | A | 311 | 17.788 | 29.682 | −1.730 | 1.00 | 0.00 | C |
| ATOM | 2275 | NE | ARG | A | 311 | 16.835 | 30.684 | −2.259 | 1.00 | 0.00 | N |
| ATOM | 2276 | CZ | ARG | A | 311 | 15.567 | 30.904 | −1.864 | 1.00 | 0.00 | C |
| ATOM | 2277 | NH1 | ARG | A | 311 | 15.074 | 30.373 | −0.750 | 1.00 | 0.00 | N1+ |
| ATOM | 2278 | NH2 | ARG | A | 311 | 14.778 | 31.691 | −2.584 | 1.00 | 0.00 | N |
| ATOM | 2279 | H | ARG | A | 311 | 19.055 | 32.902 | 0.711 | 1.00 | 0.00 | H |
| ATOM | 2280 | HA | ARG | A | 311 | 21.084 | 31.033 | 0.123 | 1.00 | 0.00 | H |
| ATOM | 2281 | 1HB | ARG | A | 311 | 18.900 | 32.246 | −1.463 | 1.00 | 0.00 | H |
| ATOM | 2282 | 2HB | ARG | A | 311 | 20.111 | 31.149 | −2.168 | 1.00 | 0.00 | H |
| ATOM | 2283 | 1HG | ARG | A | 311 | 19.507 | 29.428 | −0.488 | 1.00 | 0.00 | H |
| ATOM | 2284 | 2HG | ARG | A | 311 | 18.271 | 30.523 | 0.173 | 1.00 | 0.00 | H |
| ATOM | 2285 | 1HD | ARG | A | 311 | 18.309 | 29.258 | −2.589 | 1.00 | 0.00 | H |
| ATOM | 2286 | 2HD | ARG | A | 311 | 17.193 | 28.906 | −1.248 | 1.00 | 0.00 | H |
| ATOM | 2287 | HE | ARG | A | 311 | 17.237 | 31.239 | −3.001 | 1.00 | 0.00 | H |
| ATOM | 2288 | 1HH1 | ARG | A | 311 | 14.118 | 30.558 | −0.482 | 1.00 | 0.00 | H |
| ATOM | 2289 | 2HH1 | ARG | A | 311 | 15.656 | 29.783 | −0.172 | 1.00 | 0.00 | H |
| ATOM | 2290 | 1HH2 | ARG | A | 311 | 13.827 | 31.857 | −2.289 | 1.00 | 0.00 | H |
| ATOM | 2291 | 2HH2 | ARG | A | 311 | 15.129 | 32.123 | −3.426 | 1.00 | 0.00 | H |
| ATOM | 2292 | N | ASP | A | 312 | 22.066 | 34.029 | −0.416 | 1.00 | 0.00 | N |
| ATOM | 2293 | CA | ASP | A | 312 | 23.196 | 34.840 | −0.850 | 1.00 | 0.00 | C |
| ATOM | 2294 | C | ASP | A | 312 | 24.067 | 35.280 | 0.327 | 1.00 | 0.00 | C |
| ATOM | 2295 | O | ASP | A | 312 | 25.284 | 35.096 | 0.293 | 1.00 | 0.00 | O |
| ATOM | 2296 | CB | ASP | A | 312 | 22.750 | 36.035 | −1.693 | 1.00 | 0.00 | C |
| ATOM | 2297 | CG | ASP | A | 312 | 23.957 | 36.804 | −2.202 | 1.00 | 0.00 | C |
| ATOM | 2298 | OD1 | ASP | A | 312 | 24.970 | 36.166 | −2.562 | 1.00 | 0.00 | O |
| ATOM | 2299 | OD2 | ASP | A | 312 | 23.912 | 38.045 | −2.233 | 1.00 | 0.00 | O1− |
| ATOM | 2300 | H | ASP | A | 312 | 21.512 | 34.420 | 0.332 | 1.00 | 0.00 | H |
| ATOM | 2301 | HA | ASP | A | 312 | 23.758 | 34.302 | −1.614 | 1.00 | 0.00 | H |
| ATOM | 2302 | 1HB | ASP | A | 312 | 22.169 | 35.683 | −2.545 | 1.00 | 0.00 | H |
| ATOM | 2303 | 2HB | ASP | A | 312 | 22.137 | 36.700 | −1.086 | 1.00 | 0.00 | H |
| ATOM | 2304 | N | LEU | A | 313 | 23.465 | 35.852 | 1.370 | 1.00 | 0.00 | N |
| ATOM | 2305 | CA | LEU | A | 313 | 24.189 | 36.307 | 2.550 | 1.00 | 0.00 | C |
| ATOM | 2306 | C | LEU | A | 313 | 25.117 | 35.215 | 3.072 | 1.00 | 0.00 | C |
| ATOM | 2307 | O | LEU | A | 313 | 26.282 | 35.474 | 3.362 | 1.00 | 0.00 | O |
| ATOM | 2308 | CB | LEU | A | 313 | 23.227 | 36.694 | 3.681 | 1.00 | 0.00 | C |
| ATOM | 2309 | CG | LEU | A | 313 | 22.531 | 38.052 | 3.513 | 1.00 | 0.00 | C |
| ATOM | 2310 | CD1 | LEU | A | 313 | 21.519 | 38.189 | 4.656 | 1.00 | 0.00 | C |
| ATOM | 2311 | CD2 | LEU | A | 313 | 23.529 | 39.215 | 3.586 | 1.00 | 0.00 | C |
| ATOM | 2312 | H | LEU | A | 313 | 22.463 | 35.986 | 1.364 | 1.00 | 0.00 | H |
| ATOM | 2313 | HA | LEU | A | 313 | 24.783 | 37.185 | 2.296 | 1.00 | 0.00 | H |
| ATOM | 2314 | 1HB | LEU | A | 313 | 22.435 | 35.949 | 3.756 | 1.00 | 0.00 | H |
| ATOM | 2315 | 2HB | LEU | A | 313 | 23.772 | 36.741 | 4.623 | 1.00 | 0.00 | H |
| ATOM | 2316 | HG | LEU | A | 313 | 22.017 | 38.081 | 2.552 | 1.00 | 0.00 | H |
| ATOM | 2317 | 1HD1 | LEU | A | 313 | 21.002 | 39.145 | 4.572 | 1.00 | 0.00 | H |
| ATOM | 2318 | 2HD1 | LEU | A | 313 | 20.793 | 37.377 | 4.599 | 1.00 | 0.00 | H |
| ATOM | 2319 | 3HD1 | LEU | A | 313 | 22.041 | 38.142 | 5.612 | 1.00 | 0.00 | H |
| ATOM | 2320 | 1HD2 | LEU | A | 313 | 22.997 | 40.158 | 3.463 | 1.00 | 0.00 | H |
| ATOM | 2321 | 2HD2 | LEU | A | 313 | 24.030 | 39.204 | 4.554 | 1.00 | 0.00 | H |
| ATOM | 2322 | 3HD2 | LEU | A | 313 | 24.269 | 39.109 | 2.793 | 1.00 | 0.00 | H |
| ATOM | 2323 | N | GLU | A | 314 | 24.596 | 33.996 | 3.216 | 1.00 | 0.00 | N |
| ATOM | 2324 | CA | GLU | A | 314 | 25.322 | 32.929 | 3.875 | 1.00 | 0.00 | C |
| ATOM | 2325 | C | GLU | A | 314 | 26.442 | 32.358 | 2.998 | 1.00 | 0.00 | C |
| ATOM | 2326 | O | GLU | A | 314 | 27.173 | 31.472 | 3.454 | 1.00 | 0.00 | O |
| ATOM | 2327 | CB | GLU | A | 314 | 24.326 | 31.861 | 4.344 | 1.00 | 0.00 | C |
| ATOM | 2328 | CG | GLU | A | 314 | 23.537 | 32.367 | 5.564 | 1.00 | 0.00 | C |
| ATOM | 2329 | CD | GLU | A | 314 | 23.421 | 31.295 | 6.630 | 1.00 | 0.00 | C |
| ATOM | 2330 | OE1 | GLU | A | 314 | 24.479 | 30.922 | 7.176 | 1.00 | 0.00 | O |
| ATOM | 2331 | OE2 | GLU | A | 314 | 22.282 | 30.869 | 6.909 | 1.00 | 0.00 | O1− |
| ATOM | 2332 | H | GLU | A | 314 | 23.672 | 33.797 | 2.861 | 1.00 | 0.00 | H |
| ATOM | 2333 | HA | GLU | A | 314 | 25.682 | 33.278 | 4.843 | 1.00 | 0.00 | H |
| ATOM | 2334 | 1HB | GLU | A | 314 | 23.628 | 31.635 | 3.537 | 1.00 | 0.00 | H |
| ATOM | 2335 | 2HB | GLU | A | 314 | 24.866 | 30.955 | 4.620 | 1.00 | 0.00 | H |
| ATOM | 2336 | 1HG | GLU | A | 314 | 24.047 | 33.229 | 5.995 | 1.00 | 0.00 | H |
| ATOM | 2337 | 2HG | GLU | A | 314 | 22.533 | 32.658 | 5.254 | 1.00 | 0.00 | H |
| ATOM | 2338 | N | ARG | A | 315 | 26.594 | 32.852 | 1.766 | 1.00 | 0.00 | N |
| ATOM | 2339 | CA | ARG | A | 315 | 27.792 | 32.608 | 0.980 | 1.00 | 0.00 | C |
| ATOM | 2340 | C | ARG | A | 315 | 28.940 | 33.437 | 1.566 | 1.00 | 0.00 | C |
| ATOM | 2341 | O | ARG | A | 315 | 30.093 | 33.020 | 1.525 | 1.00 | 0.00 | O |
| ATOM | 2342 | CB | ARG | A | 315 | 27.587 | 33.030 | −0.485 | 1.00 | 0.00 | C |
| ATOM | 2343 | CG | ARG | A | 315 | 26.358 | 32.416 | −1.172 | 1.00 | 0.00 | C |
| ATOM | 2344 | CD | ARG | A | 315 | 26.188 | 33.043 | −2.567 | 1.00 | 0.00 | C |
| ATOM | 2345 | NE | ARG | A | 315 | 24.855 | 32.769 | −3.133 | 1.00 | 0.00 | N |
| ATOM | 2346 | CZ | ARG | A | 315 | 24.233 | 33.493 | −4.079 | 1.00 | 0.00 | C |
| ATOM | 2347 | NH1 | ARG | A | 315 | 24.816 | 34.563 | −4.614 | 1.00 | 0.00 | N1+ |
| ATOM | 2348 | NH2 | ARG | A | 315 | 23.009 | 33.136 | −4.457 | 1.00 | 0.00 | N |
| ATOM | 2349 | H | ARG | A | 315 | 25.864 | 33.414 | 1.353 | 1.00 | 0.00 | H |
| ATOM | 2350 | HA | ARG | A | 315 | 28.052 | 31.550 | 1.032 | 1.00 | 0.00 | H |
| ATOM | 2351 | 1HB | ARG | A | 315 | 27.466 | 34.112 | −0.538 | 1.00 | 0.00 | H |
| ATOM | 2352 | 2HB | ARG | A | 315 | 28.455 | 32.733 | −1.074 | 1.00 | 0.00 | H |
| ATOM | 2353 | 1HG | ARG | A | 315 | 26.496 | 31.339 | −1.271 | 1.00 | 0.00 | H |
| ATOM | 2354 | 2HG | ARG | A | 315 | 25.469 | 32.615 | −0.573 | 1.00 | 0.00 | H |
| ATOM | 2355 | 1HD | ARG | A | 315 | 26.315 | 34.124 | −2.498 | 1.00 | 0.00 | H |
| ATOM | 2356 | 2HD | ARG | A | 315 | 26.937 | 32.632 | −3.244 | 1.00 | 0.00 | H |
| ATOM | 2357 | HE | ARG | A | 315 | 24.404 | 31.953 | −2.745 | 1.00 | 0.00 | H |
| ATOM | 2358 | 1HH1 | ARG | A | 315 | 24.336 | 35.097 | −5.324 | 1.00 | 0.00 | H |
| ATOM | 2359 | 2HH1 | ARG | A | 315 | 25.738 | 34.841 | −4.309 | 1.00 | 0.00 | H |
| ATOM | 2360 | 1HH2 | ARG | A | 315 | 22.524 | 33.667 | −5.167 | 1.00 | 0.00 | H |
| ATOM | 2361 | 2HH2 | ARG | A | 315 | 22.563 | 32.334 | −4.035 | 1.00 | 0.00 | H |
| ATOM | 2362 | N | GLY | A | 316 | 28.619 | 34.645 | 2.025 | 1.00 | 0.00 | N |
| ATOM | 2363 | CA | GLY | A | 316 | 29.575 | 35.645 | 2.465 | 1.00 | 0.00 | C |
| ATOM | 2364 | C | GLY | A | 316 | 28.996 | 37.028 | 2.158 | 1.00 | 0.00 | C |
| ATOM | 2365 | O | GLY | A | 316 | 28.255 | 37.173 | 1.183 | 1.00 | 0.00 | O |
| ATOM | 2366 | H | GLY | A | 316 | 27.650 | 34.922 | 2.086 | 1.00 | 0.00 | H |
| ATOM | 2367 | 1HA | GLY | A | 316 | 29.745 | 35.539 | 3.536 | 1.00 | 0.00 | H |
| ATOM | 2368 | 2HA | GLY | A | 316 | 30.517 | 35.507 | 1.933 | 1.00 | 0.00 | H |
| ATOM | 2369 | N | CYS | A | 317 | 29.325 | 38.057 | 2.941 | 1.00 | 0.00 | N |
| ATOM | 2370 | CA | CYS | A | 317 | 28.883 | 39.428 | 2.711 | 1.00 | 0.00 | C |
| ATOM | 2371 | C | CYS | A | 317 | 29.800 | 40.358 | 3.506 | 1.00 | 0.00 | C |
| ATOM | 2372 | O | CYS | A | 317 | 29.919 | 40.199 | 4.718 | 1.00 | 0.00 | O |
| ATOM | 2373 | CB | CYS | A | 317 | 27.417 | 39.563 | 3.144 | 1.00 | 0.00 | C |
| ATOM | 2374 | SG | CYS | A | 317 | 26.883 | 41.294 | 3.166 | 1.00 | 0.00 | S |
| ATOM | 2375 | H | CYS | A | 317 | 29.914 | 37.909 | 3.748 | 1.00 | 0.00 | H |
| ATOM | 2376 | HA | CYS | A | 317 | 28.915 | 39.647 | 1.644 | 1.00 | 0.00 | H |
| ATOM | 2377 | 1HB | CYS | A | 317 | 26.780 | 39.016 | 2.449 | 1.00 | 0.00 | H |
| ATOM | 2378 | 2HB | CYS | A | 317 | 27.295 | 39.154 | 4.147 | 1.00 | 0.00 | H |
| ATOM | 2379 | HG | CYS | A | 317 | 25.830 | 41.349 | 3.481 | 1.00 | 0.00 | H |
| ATOM | 2380 | N | HIS | A | 318 | 30.474 | 41.300 | 2.834 | 1.00 | 0.00 | N |
| ATOM | 2381 | CA | HIS | A | 318 | 31.444 | 42.193 | 3.470 | 1.00 | 0.00 | C |
| ATOM | 2382 | C | HIS | A | 318 | 30.885 | 43.617 | 3.531 | 1.00 | 0.00 | C |
| ATOM | 2383 | O | HIS | A | 318 | 31.498 | 44.502 | 4.124 | 1.00 | 0.00 | O |
| ATOM | 2384 | CB | HIS | A | 318 | 32.753 | 42.250 | 2.666 | 1.00 | 0.00 | C |
| ATOM | 2385 | CG | HIS | A | 318 | 33.410 | 40.942 | 2.291 | 1.00 | 0.00 | C |
| ATOM | 2386 | CD2 | HIS | A | 318 | 33.040 | 39.623 | 2.415 | 1.00 | 0.00 | C |
| ATOM | 2387 | ND1 | HIS | A | 318 | 34.653 | 40.909 | 1.664 | 1.00 | 0.00 | N |
| ATOM | 2388 | CE1 | HIS | A | 318 | 34.952 | 39.628 | 1.393 | 1.00 | 0.00 | C |
| ATOM | 2389 | NE2 | HIS | A | 318 | 33.990 | 38.814 | 1.816 | 1.00 | 0.00 | N |
| ATOM | 2390 | H | HIS | A | 318 | 30.323 | 41.419 | 1.843 | 1.00 | 0.00 | H |
| ATOM | 2391 | HA | HIS | A | 318 | 31.632 | 41.859 | 4.490 | 1.00 | 0.00 | H |
| ATOM | 2392 | 1HB | HIS | A | 318 | 32.577 | 42.761 | 1.719 | 1.00 | 0.00 | H |
| ATOM | 2393 | 2HB | HIS | A | 318 | 33.507 | 42.793 | 3.236 | 1.00 | 0.00 | H |
| ATOM | 2394 | HD2 | HIS | A | 318 | 32.124 | 39.365 | 2.925 | 1.00 | 0.00 | H |
| ATOM | 2395 | HD1 | HIS | A | 318 | 35.156 | 41.769 | 1.494 | 1.00 | 0.00 | H |
| ATOM | 2396 | HE1 | HIS | A | 318 | 35.877 | 39.384 | 0.892 | 1.00 | 0.00 | H |
| ATOM | 2397 | N | LEU | A | 319 | 29.761 | 43.855 | 2.861 | 1.00 | 0.00 | N |
| ATOM | 2398 | CA | LEU | A | 319 | 29.164 | 45.163 | 2.645 | 1.00 | 0.00 | C |
| ATOM | 2399 | C | LEU | A | 319 | 27.727 | 44.879 | 2.219 | 1.00 | 0.00 | C |
| ATOM | 2400 | O | LEU | A | 319 | 27.492 | 43.890 | 1.521 | 1.00 | 0.00 | O |
| ATOM | 2401 | CB | LEU | A | 319 | 29.957 | 45.938 | 1.577 | 1.00 | 0.00 | C |
| ATOM | 2402 | CG | LEU | A | 319 | 29.459 | 47.383 | 1.376 | 1.00 | 0.00 | C |
| ATOM | 2403 | CD1 | LEU | A | 319 | 30.645 | 48.298 | 1.041 | 1.00 | 0.00 | C |
| ATOM | 2404 | CD2 | LEU | A | 319 | 28.433 | 47.494 | 0.240 | 1.00 | 0.00 | C |
| ATOM | 2405 | H | LEU | A | 319 | 29.247 | 43.090 | 2.449 | 1.00 | 0.00 | H |
| ATOM | 2406 | HA | LEU | A | 319 | 29.227 | 45.748 | 3.562 | 1.00 | 0.00 | H |
| ATOM | 2407 | 1HB | LEU | A | 319 | 31.005 | 45.990 | 1.870 | 1.00 | 0.00 | H |
| ATOM | 2408 | 2HB | LEU | A | 319 | 29.872 | 45.426 | 0.619 | 1.00 | 0.00 | H |
| ATOM | 2409 | HG | LEU | A | 319 | 28.976 | 47.732 | 2.289 | 1.00 | 0.00 | H |
| ATOM | 2410 | 1HD1 | LEU | A | 319 | 30.288 | 49.318 | 0.899 | 1.00 | 0.00 | H |
| ATOM | 2411 | 2HD1 | LEU | A | 319 | 31.365 | 48.275 | 1.858 | 1.00 | 0.00 | H |
| ATOM | 2412 | 3HD1 | LEU | A | 319 | 31.124 | 47.951 | 0.125 | 1.00 | 0.00 | H |
| ATOM | 2413 | 1HD2 | LEU | A | 319 | 28.114 | 48.532 | 0.138 | 1.00 | 0.00 | H |
| ATOM | 2414 | 2HD2 | LEU | A | 319 | 28.887 | 47.161 | −0.694 | 1.00 | 0.00 | H |
| ATOM | 2415 | 3HD2 | LEU | A | 319 | 27.569 | 46.870 | 0.467 | 1.00 | 0.00 | H |
| ATOM | 2416 | N | LEU | A | 320 | 26.768 | 45.678 | 2.676 | 1.00 | 0.00 | N |
| ATOM | 2417 | CA | LEU | A | 320 | 25.365 | 45.306 | 2.623 | 1.00 | 0.00 | C |
| ATOM | 2418 | C | LEU | A | 320 | 24.502 | 46.547 | 2.448 | 1.00 | 0.00 | C |
| ATOM | 2419 | O | LEU | A | 320 | 24.774 | 47.571 | 3.073 | 1.00 | 0.00 | O |
| ATOM | 2420 | CB | LEU | A | 320 | 25.026 | 44.603 | 3.940 | 1.00 | 0.00 | C |
| ATOM | 2421 | CG | LEU | A | 320 | 23.575 | 44.115 | 4.057 | 1.00 | 0.00 | C |
| ATOM | 2422 | CD1 | LEU | A | 320 | 23.172 | 43.159 | 2.930 | 1.00 | 0.00 | C |
| ATOM | 2423 | CD2 | LEU | A | 320 | 23.466 | 43.354 | 5.376 | 1.00 | 0.00 | C |
| ATOM | 2424 | H | LEU | A | 320 | 27.005 | 46.575 | 3.075 | 1.00 | 0.00 | H |
| ATOM | 2425 | HA | LEU | A | 320 | 25.199 | 44.629 | 1.785 | 1.00 | 0.00 | H |
| ATOM | 2426 | 1HB | LEU | A | 320 | 25.662 | 43.725 | 4.059 | 1.00 | 0.00 | H |
| ATOM | 2427 | 2HB | LEU | A | 320 | 25.194 | 45.287 | 4.772 | 1.00 | 0.00 | H |
| ATOM | 2428 | HG | LEU | A | 320 | 22.898 | 44.967 | 3.994 | 1.00 | 0.00 | H |
| ATOM | 2429 | 1HD1 | LEU | A | 320 | 22.136 | 42.850 | 3.069 | 1.00 | 0.00 | H |
| ATOM | 2430 | 2HD1 | LEU | A | 320 | 23.275 | 43.664 | 1.970 | 1.00 | 0.00 | H |
| ATOM | 2431 | 3HD1 | LEU | A | 320 | 23.818 | 42.281 | 2.949 | 1.00 | 0.00 | H |
| ATOM | 2432 | 1HD2 | LEU | A | 320 | 22.447 | 42.987 | 5.501 | 1.00 | 0.00 | H |
| ATOM | 2433 | 2HD2 | LEU | A | 320 | 24.157 | 42.511 | 5.367 | 1.00 | 0.00 | H |
| ATOM | 2434 | 3HD2 | LEU | A | 320 | 23.716 | 44.020 | 6.202 | 1.00 | 0.00 | H |
| ATOM | 2435 | N | VAL | A | 321 | 23.452 | 46.431 | 1.637 | 1.00 | 0.00 | N |
| ATOM | 2436 | CA | VAL | A | 321 | 22.434 | 47.453 | 1.469 | 1.00 | 0.00 | C |
| ATOM | 2437 | C | VAL | A | 321 | 21.093 | 46.812 | 1.845 | 1.00 | 0.00 | C |
| ATOM | 2438 | O | VAL | A | 321 | 20.824 | 45.687 | 1.413 | 1.00 | 0.00 | O |
| ATOM | 2439 | CB | VAL | A | 321 | 22.436 | 47.962 | 0.018 | 1.00 | 0.00 | C |
| ATOM | 2440 | CG1 | VAL | A | 321 | 21.572 | 49.223 | −0.067 | 1.00 | 0.00 | C |
| ATOM | 2441 | CG2 | VAL | A | 321 | 23.849 | 48.280 | −0.496 | 1.00 | 0.00 | C |
| ATOM | 2442 | H | VAL | A | 321 | 23.327 | 45.591 | 1.090 | 1.00 | 0.00 | H |
| ATOM | 2443 | HA | VAL | A | 321 | 22.669 | 48.308 | 2.103 | 1.00 | 0.00 | H |
| ATOM | 2444 | HB | VAL | A | 321 | 22.052 | 47.183 | −0.641 | 1.00 | 0.00 | H |
| ATOM | 2445 | 1HG1 | VAL | A | 321 | 21.567 | 49.592 | −1.093 | 1.00 | 0.00 | H |
| ATOM | 2446 | 2HG1 | VAL | A | 321 | 20.553 | 48.987 | 0.238 | 1.00 | 0.00 | H |
| ATOM | 2447 | 3HG1 | VAL | A | 321 | 21.981 | 49.989 | 0.591 | 1.00 | 0.00 | H |
| ATOM | 2448 | 1HG2 | VAL | A | 321 | 23.790 | 48.635 | −1.525 | 1.00 | 0.00 | H |
| ATOM | 2449 | 2HG2 | VAL | A | 321 | 24.298 | 49.052 | 0.129 | 1.00 | 0.00 | H |
| ATOM | 2450 | 3HG2 | VAL | A | 321 | 24.461 | 47.379 | −0.457 | 1.00 | 0.00 | H |
| ATOM | 2451 | N | ALA | A | 322 | 20.264 | 47.466 | 2.662 | 1.00 | 0.00 | N |
| ATOM | 2452 | CA | ALA | A | 322 | 19.098 | 46.813 | 3.244 | 1.00 | 0.00 | C |
| ATOM | 2453 | C | ALA | A | 322 | 17.926 | 47.764 | 3.486 | 1.00 | 0.00 | C |
| ATOM | 2454 | O | ALA | A | 322 | 18.103 | 48.840 | 4.048 | 1.00 | 0.00 | O |
| ATOM | 2455 | CB | ALA | A | 322 | 19.506 | 46.165 | 4.567 | 1.00 | 0.00 | C |
| ATOM | 2456 | H | ALA | A | 322 | 20.439 | 48.435 | 2.889 | 1.00 | 0.00 | H |
| ATOM | 2457 | HA | ALA | A | 322 | 18.769 | 46.005 | 2.590 | 1.00 | 0.00 | H |
| ATOM | 2458 | 1HB | ALA | A | 322 | 18.642 | 45.673 | 5.012 | 1.00 | 0.00 | H |
| ATOM | 2459 | 2HB | ALA | A | 322 | 20.289 | 45.429 | 4.385 | 1.00 | 0.00 | H |
| ATOM | 2460 | 3HB | ALA | A | 322 | 19.878 | 46.931 | 5.247 | 1.00 | 0.00 | H |
| ATOM | 2461 | N | THR | A | 323 | 16.710 | 47.332 | 3.147 | 1.00 | 0.00 | N |
| ATOM | 2462 | CA | THR | A | 323 | 15.513 | 47.989 | 3.643 | 1.00 | 0.00 | C |
| ATOM | 2463 | C | THR | A | 323 | 15.367 | 47.574 | 5.120 | 1.00 | 0.00 | C |
| ATOM | 2464 | O | THR | A | 323 | 15.319 | 46.365 | 5.365 | 1.00 | 0.00 | O |
| ATOM | 2465 | CB | THR | A | 323 | 14.306 | 47.518 | 2.811 | 1.00 | 0.00 | C |
| ATOM | 2466 | CG2 | THR | A | 323 | 14.396 | 48.006 | 1.361 | 1.00 | 0.00 | C |
| ATOM | 2467 | OG1 | THR | A | 323 | 14.239 | 46.100 | 2.807 | 1.00 | 0.00 | O |
| ATOM | 2468 | H | THR | A | 323 | 16.610 | 46.534 | 2.535 | 1.00 | 0.00 | H |
| ATOM | 2469 | HA | THR | A | 323 | 15.626 | 49.069 | 3.552 | 1.00 | 0.00 | H |
| ATOM | 2470 | HB | THR | A | 323 | 13.387 | 47.899 | 3.256 | 1.00 | 0.00 | H |
| ATOM | 2471 | 1HG2 | THR | A | 323 | 13.528 | 47.654 | 0.804 | 1.00 | 0.00 | H |
| ATOM | 2472 | 2HG2 | THR | A | 323 | 14.421 | 49.096 | 1.345 | 1.00 | 0.00 | H |
| ATOM | 2473 | 3HG2 | THR | A | 323 | 15.304 | 47.616 | 0.901 | 1.00 | 0.00 | H |
| ATOM | 2474 | HG1 | THR | A | 323 | 13.484 | 45.816 | 2.286 | 1.00 | 0.00 | H |
| ATOM | 2475 | N | PRO | A | 324 | 15.269 | 48.496 | 6.095 | 1.00 | 0.00 | N |
| ATOM | 2476 | CA | PRO | A | 324 | 15.544 | 48.216 | 7.502 | 1.00 | 0.00 | C |
| ATOM | 2477 | C | PRO | A | 324 | 14.872 | 46.960 | 8.068 | 1.00 | 0.00 | C |
| ATOM | 2478 | O | PRO | A | 324 | 15.510 | 46.140 | 8.729 | 1.00 | 0.00 | O |
| ATOM | 2479 | CB | PRO | A | 324 | 15.093 | 49.474 | 8.242 | 1.00 | 0.00 | C |
| ATOM | 2480 | CG | PRO | A | 324 | 15.494 | 50.578 | 7.268 | 1.00 | 0.00 | C |
| ATOM | 2481 | CD | PRO | A | 324 | 15.309 | 49.942 | 5.885 | 1.00 | 0.00 | C |
| ATOM | 2482 | HA | PRO | A | 324 | 16.620 | 48.132 | 7.653 | 1.00 | 0.00 | H |
| ATOM | 2483 | 1HB | PRO | A | 324 | 14.017 | 49.428 | 8.415 | 1.00 | 0.00 | H |
| ATOM | 2484 | 2HB | PRO | A | 324 | 15.611 | 49.540 | 9.198 | 1.00 | 0.00 | H |
| ATOM | 2485 | 1HG | PRO | A | 324 | 14.847 | 51.443 | 7.411 | 1.00 | 0.00 | H |
| ATOM | 2486 | 2HG | PRO | A | 324 | 16.530 | 50.866 | 7.451 | 1.00 | 0.00 | H |
| ATOM | 2487 | 1HD | PRO | A | 324 | 16.141 | 50.191 | 5.227 | 1.00 | 0.00 | H |
| ATOM | 2488 | 2HD | PRO | A | 324 | 14.375 | 50.269 | 5.429 | 1.00 | 0.00 | H |
| ATOM | 2489 | N | GLY | A | 325 | 13.578 | 46.794 | 7.797 | 1.00 | 0.00 | N |
| ATOM | 2490 | CA | GLY | A | 325 | 12.819 | 45.667 | 8.312 | 1.00 | 0.00 | C |
| ATOM | 2491 | C | GLY | A | 325 | 13.486 | 44.338 | 7.947 | 1.00 | 0.00 | C |
| ATOM | 2492 | O | GLY | A | 325 | 13.493 | 43.402 | 8.742 | 1.00 | 0.00 | O |
| ATOM | 2493 | H | GLY | A | 325 | 13.094 | 47.463 | 7.216 | 1.00 | 0.00 | H |
| ATOM | 2494 | 1HA | GLY | A | 325 | 12.756 | 45.737 | 9.398 | 1.00 | 0.00 | H |
| ATOM | 2495 | 2HA | GLY | A | 325 | 11.815 | 45.680 | 7.888 | 1.00 | 0.00 | H |
| ATOM | 2496 | N | ARG | A | 326 | 14.086 | 44.261 | 6.756 | 1.00 | 0.00 | N |
| ATOM | 2497 | CA | ARG | A | 326 | 14.723 | 43.054 | 6.254 | 1.00 | 0.00 | C |
| ATOM | 2498 | C | ARG | A | 326 | 16.220 | 43.070 | 6.585 | 1.00 | 0.00 | C |
| ATOM | 2499 | O | ARG | A | 326 | 17.026 | 42.392 | 5.949 | 1.00 | 0.00 | O |
| ATOM | 2500 | CB | ARG | A | 326 | 14.394 | 42.886 | 4.763 | 1.00 | 0.00 | C |
| ATOM | 2501 | CG | ARG | A | 326 | 15.010 | 41.669 | 4.053 | 1.00 | 0.00 | C |
| ATOM | 2502 | CD | ARG | A | 326 | 15.002 | 40.352 | 4.848 | 1.00 | 0.00 | C |
| ATOM | 2503 | NE | ARG | A | 326 | 13.659 | 39.845 | 5.151 | 1.00 | 0.00 | N |
| ATOM | 2504 | CZ | ARG | A | 326 | 12.806 | 39.257 | 4.310 | 1.00 | 0.00 | C |
| ATOM | 2505 | NH1 | ARG | A | 326 | 13.064 | 39.216 | 3.005 | 1.00 | 0.00 | N1+ |
| ATOM | 2506 | NH2 | ARG | A | 326 | 11.711 | 38.703 | 4.818 | 1.00 | 0.00 | N |
| ATOM | 2507 | H | ARG | A | 326 | 14.113 | 45.069 | 6.151 | 1.00 | 0.00 | H |
| ATOM | 2508 | HA | ARG | A | 326 | 14.184 | 42.179 | 6.619 | 1.00 | 0.00 | H |
| ATOM | 2509 | 1HB | ARG | A | 326 | 13.316 | 42.788 | 4.637 | 1.00 | 0.00 | H |
| ATOM | 2510 | 2HB | ARG | A | 326 | 14.745 | 43.759 | 4.212 | 1.00 | 0.00 | H |
| ATOM | 2511 | 1HG | ARG | A | 326 | 14.464 | 41.470 | 3.131 | 1.00 | 0.00 | H |
| ATOM | 2512 | 2HG | ARG | A | 326 | 16.055 | 41.875 | 3.819 | 1.00 | 0.00 | H |
| ATOM | 2513 | 1HD | ARG | A | 326 | 15.514 | 39.578 | 4.275 | 1.00 | 0.00 | H |
| ATOM | 2514 | 2HD | ARG | A | 326 | 15.513 | 40.497 | 5.799 | 1.00 | 0.00 | H |
| ATOM | 2515 | HE | ARG | A | 326 | 13.401 | 39.983 | 6.118 | 1.00 | 0.00 | H |
| ATOM | 2516 | 1HH1 | ARG | A | 326 | 12.414 | 38.769 | 2.375 | 1.00 | 0.00 | H |
| ATOM | 2517 | 2HH1 | ARG | A | 326 | 13.911 | 39.632 | 2.644 | 1.00 | 0.00 | H |
| ATOM | 2518 | 1HH2 | ARG | A | 326 | 11.045 | 38.250 | 4.209 | 1.00 | 0.00 | H |
| ATOM | 2519 | 2HH2 | ARG | A | 326 | 11.544 | 38.735 | 5.814 | 1.00 | 0.00 | H |
| ATOM | 2520 | N | LEU | A | 327 | 16.597 | 43.806 | 7.626 | 1.00 | 0.00 | N |
| ATOM | 2521 | CA | LEU | A | 327 | 17.877 | 43.651 | 8.284 | 1.00 | 0.00 | C |
| ATOM | 2522 | C | LEU | A | 327 | 17.606 | 43.095 | 9.677 | 1.00 | 0.00 | C |
| ATOM | 2523 | O | LEU | A | 327 | 18.223 | 42.108 | 10.074 | 1.00 | 0.00 | O |
| ATOM | 2524 | CB | LEU | A | 327 | 18.624 | 44.985 | 8.323 | 1.00 | 0.00 | C |
| ATOM | 2525 | CG | LEU | A | 327 | 20.113 | 44.789 | 8.640 | 1.00 | 0.00 | C |
| ATOM | 2526 | CD1 | LEU | A | 327 | 20.883 | 44.162 | 7.473 | 1.00 | 0.00 | C |
| ATOM | 2527 | CD2 | LEU | A | 327 | 20.733 | 46.152 | 8.924 | 1.00 | 0.00 | C |
| ATOM | 2528 | H | LEU | A | 327 | 15.979 | 44.514 | 7.994 | 1.00 | 0.00 | H |
| ATOM | 2529 | HA | LEU | A | 327 | 18.519 | 43.005 | 7.685 | 1.00 | 0.00 | H |
| ATOM | 2530 | 1HB | LEU | A | 327 | 18.541 | 45.477 | 7.354 | 1.00 | 0.00 | H |
| ATOM | 2531 | 2HB | LEU | A | 327 | 18.190 | 45.624 | 9.092 | 1.00 | 0.00 | H |
| ATOM | 2532 | HG | LEU | A | 327 | 20.219 | 44.118 | 9.492 | 1.00 | 0.00 | H |
| ATOM | 2533 | 1HD1 | LEU | A | 327 | 21.931 | 44.045 | 7.749 | 1.00 | 0.00 | H |
| ATOM | 2534 | 2HD1 | LEU | A | 327 | 20.458 | 43.186 | 7.239 | 1.00 | 0.00 | H |
| ATOM | 2535 | 3HD1 | LEU | A | 327 | 20.808 | 44.809 | 6.599 | 1.00 | 0.00 | H |
| ATOM | 2536 | 1HD2 | LEU | A | 327 | 21.792 | 46.031 | 9.151 | 1.00 | 0.00 | H |
| ATOM | 2537 | 2HD2 | LEU | A | 327 | 20.621 | 46.792 | 8.048 | 1.00 | 0.00 | H |
| ATOM | 2538 | 3HD2 | LEU | A | 327 | 20.229 | 46.611 | 9.775 | 1.00 | 0.00 | H |
| ATOM | 2539 | N | VAL | A | 328 | 16.643 | 43.679 | 10.392 | 1.00 | 0.00 | N |
| ATOM | 2540 | CA | VAL | A | 328 | 16.180 | 43.141 | 11.665 | 1.00 | 0.00 | C |
| ATOM | 2541 | C | VAL | A | 328 | 15.821 | 41.661 | 11.476 | 1.00 | 0.00 | C |
| ATOM | 2542 | O | VAL | A | 328 | 16.260 | 40.799 | 12.239 | 1.00 | 0.00 | O |
| ATOM | 2543 | CB | VAL | A | 328 | 14.993 | 43.968 | 12.194 | 1.00 | 0.00 | C |
| ATOM | 2544 | CG1 | VAL | A | 328 | 14.470 | 43.384 | 13.510 | 1.00 | 0.00 | C |
| ATOM | 2545 | CG2 | VAL | A | 328 | 15.408 | 45.425 | 12.438 | 1.00 | 0.00 | C |
| ATOM | 2546 | H | VAL | A | 328 | 16.207 | 44.526 | 10.055 | 1.00 | 0.00 | H |
| ATOM | 2547 | HA | VAL | A | 328 | 16.946 | 43.294 | 12.425 | 1.00 | 0.00 | H |
| ATOM | 2548 | HB | VAL | A | 328 | 14.191 | 43.966 | 11.456 | 1.00 | 0.00 | H |
| ATOM | 2549 | 1HG1 | VAL | A | 328 | 13.632 | 43.983 | 13.867 | 1.00 | 0.00 | H |
| ATOM | 2550 | 2HG1 | VAL | A | 328 | 14.139 | 42.358 | 13.347 | 1.00 | 0.00 | H |
| ATOM | 2551 | 3HG1 | VAL | A | 328 | 15.266 | 43.394 | 14.255 | 1.00 | 0.00 | H |
| ATOM | 2552 | 1HG2 | VAL | A | 328 | 14.553 | 45.989 | 12.811 | 1.00 | 0.00 | H |
| ATOM | 2553 | 2HG2 | VAL | A | 328 | 16.212 | 45.456 | 13.174 | 1.00 | 0.00 | H |
| ATOM | 2554 | 3HG2 | VAL | A | 328 | 15.754 | 45.867 | 11.504 | 1.00 | 0.00 | H |
| ATOM | 2555 | N | ASP | A | 329 | 15.077 | 41.370 | 10.404 | 1.00 | 0.00 | N |
| ATOM | 2556 | CA | ASP | A | 329 | 14.580 | 40.037 | 10.081 | 1.00 | 0.00 | C |
| ATOM | 2557 | C | ASP | A | 329 | 15.716 | 39.037 | 9.810 | 1.00 | 0.00 | C |
| ATOM | 2558 | O | ASP | A | 329 | 15.490 | 37.837 | 9.679 | 1.00 | 0.00 | O |
| ATOM | 2559 | CB | ASP | A | 329 | 13.657 | 40.159 | 8.861 | 1.00 | 0.00 | C |
| ATOM | 2560 | CG | ASP | A | 329 | 12.850 | 38.917 | 8.533 | 1.00 | 0.00 | C |
| ATOM | 2561 | OD1 | ASP | A | 329 | 12.418 | 38.234 | 9.483 | 1.00 | 0.00 | O |
| ATOM | 2562 | OD2 | ASP | A | 329 | 12.615 | 38.707 | 7.321 | 1.00 | 0.00 | O1− |
| ATOM | 2563 | H | ASP | A | 329 | 14.824 | 42.098 | 9.751 | 1.00 | 0.00 | H |
| ATOM | 2564 | HA | ASP | A | 329 | 13.953 | 39.676 | 10.896 | 1.00 | 0.00 | H |
| ATOM | 2565 | 1HB | ASP | A | 329 | 12.937 | 40.960 | 9.027 | 1.00 | 0.00 | H |
| ATOM | 2566 | 2HB | ASP | A | 329 | 14.252 | 40.384 | 7.975 | 1.00 | 0.00 | H |
| ATOM | 2567 | N | MET | A | 330 | 16.950 | 39.526 | 9.678 | 1.00 | 0.00 | N |
| ATOM | 2568 | CA | MET | A | 330 | 18.132 | 38.723 | 9.423 | 1.00 | 0.00 | C |
| ATOM | 2569 | C | MET | A | 330 | 19.016 | 38.672 | 10.668 | 1.00 | 0.00 | C |
| ATOM | 2570 | O | MET | A | 330 | 19.720 | 37.683 | 10.881 | 1.00 | 0.00 | O |
| ATOM | 2571 | CB | MET | A | 330 | 18.898 | 39.340 | 8.252 | 1.00 | 0.00 | C |
| ATOM | 2572 | CG | MET | A | 330 | 18.206 | 39.083 | 6.916 | 1.00 | 0.00 | C |
| ATOM | 2573 | SD | MET | A | 330 | 18.359 | 37.367 | 6.355 | 1.00 | 0.00 | S |
| ATOM | 2574 | CE | MET | A | 330 | 17.908 | 37.579 | 4.621 | 1.00 | 0.00 | C |
| ATOM | 2575 | H | MET | A | 330 | 17.110 | 40.520 | 9.754 | 1.00 | 0.00 | H |
| ATOM | 2576 | HA | MET | A | 330 | 17.833 | 37.715 | 9.139 | 1.00 | 0.00 | H |
| ATOM | 2577 | 1HB | MET | A | 330 | 18.972 | 40.418 | 8.393 | 1.00 | 0.00 | H |
| ATOM | 2578 | 2HB | MET | A | 330 | 19.898 | 38.910 | 8.204 | 1.00 | 0.00 | H |
| ATOM | 2579 | 1HG | MET | A | 330 | 17.143 | 39.307 | 7.007 | 1.00 | 0.00 | H |
| ATOM | 2580 | 2HG | MET | A | 330 | 18.646 | 39.721 | 6.149 | 1.00 | 0.00 | H |
| ATOM | 2581 | 1HE | MET | A | 330 | 17.949 | 36.615 | 4.114 | 1.00 | 0.00 | H |
| ATOM | 2582 | 2HE | MET | A | 330 | 16.898 | 37.982 | 4.555 | 1.00 | 0.00 | H |
| ATOM | 2583 | 3HE | MET | A | 330 | 18.606 | 38.269 | 4.146 | 1.00 | 0.00 | H |
| ATOM | 2584 | N | MET | A | 331 | 19.003 | 39.736 | 11.466 | 1.00 | 0.00 | N |
| ATOM | 2585 | CA | MET | A | 331 | 19.748 | 39.833 | 12.710 | 1.00 | 0.00 | C |
| ATOM | 2586 | C | MET | A | 331 | 19.180 | 38.824 | 13.700 | 1.00 | 0.00 | C |
| ATOM | 2587 | O | MET | A | 331 | 19.903 | 37.962 | 14.201 | 1.00 | 0.00 | O |
| ATOM | 2588 | CB | MET | A | 331 | 19.667 | 41.263 | 13.256 | 1.00 | 0.00 | C |
| ATOM | 2589 | CG | MET | A | 331 | 20.519 | 42.211 | 12.406 | 1.00 | 0.00 | C |
| ATOM | 2590 | SD | MET | A | 331 | 20.126 | 43.969 | 12.567 | 1.00 | 0.00 | S |
| ATOM | 2591 | CE | MET | A | 331 | 20.310 | 44.138 | 14.354 | 1.00 | 0.00 | C |
| ATOM | 2592 | H | MET | A | 331 | 18.451 | 40.545 | 11.220 | 1.00 | 0.00 | H |
| ATOM | 2593 | HA | MET | A | 331 | 20.806 | 39.657 | 12.516 | 1.00 | 0.00 | H |
| ATOM | 2594 | 1HB | MET | A | 331 | 18.632 | 41.603 | 13.234 | 1.00 | 0.00 | H |
| ATOM | 2595 | 2HB | MET | A | 331 | 20.034 | 41.282 | 14.282 | 1.00 | 0.00 | H |
| ATOM | 2596 | 1HG | MET | A | 331 | 21.568 | 42.105 | 12.684 | 1.00 | 0.00 | H |
| ATOM | 2597 | 2HG | MET | A | 331 | 20.396 | 41.964 | 11.352 | 1.00 | 0.00 | H |
| ATOM | 2598 | 1HE | MET | A | 331 | 20.105 | 45.169 | 14.645 | 1.00 | 0.00 | H |
| ATOM | 2599 | 2HE | MET | A | 331 | 19.609 | 43.472 | 14.857 | 1.00 | 0.00 | H |
| ATOM | 2600 | 3HE | MET | A | 331 | 21.329 | 43.877 | 14.641 | 1.00 | 0.00 | H |
| ATOM | 2601 | N | GLU | A | 332 | 17.865 | 38.884 | 13.920 | 1.00 | 0.00 | N |
| ATOM | 2602 | CA | GLU | A | 332 | 17.158 | 38.012 | 14.846 | 1.00 | 0.00 | C |
| ATOM | 2603 | C | GLU | A | 332 | 17.308 | 36.536 | 14.474 | 1.00 | 0.00 | C |
| ATOM | 2604 | O | GLU | A | 332 | 16.894 | 35.655 | 15.223 | 1.00 | 0.00 | O |
| ATOM | 2605 | CB | GLU | A | 332 | 15.669 | 38.344 | 14.788 | 1.00 | 0.00 | C |
| ATOM | 2606 | CG | GLU | A | 332 | 15.339 | 39.729 | 15.336 | 1.00 | 0.00 | C |
| ATOM | 2607 | CD | GLU | A | 332 | 13.868 | 40.056 | 15.168 | 1.00 | 0.00 | C |
| ATOM | 2608 | OE1 | GLU | A | 332 | 13.147 | 39.242 | 14.544 | 1.00 | 0.00 | O |
| ATOM | 2609 | OE2 | GLU | A | 332 | 13.508 | 41.130 | 15.683 | 1.00 | 0.00 | O1− |
| ATOM | 2610 | H | GLU | A | 332 | 17.302 | 39.565 | 13.431 | 1.00 | 0.00 | H |
| ATOM | 2611 | HA | GLU | A | 332 | 17.574 | 38.128 | 15.847 | 1.00 | 0.00 | H |
| ATOM | 2612 | 1HB | GLU | A | 332 | 15.329 | 38.312 | 13.752 | 1.00 | 0.00 | H |
| ATOM | 2613 | 2HB | GLU | A | 332 | 15.110 | 37.617 | 15.376 | 1.00 | 0.00 | H |
| ATOM | 2614 | 1HG | GLU | A | 332 | 15.582 | 39.767 | 16.398 | 1.00 | 0.00 | H |
| ATOM | 2615 | 2HG | GLU | A | 332 | 15.922 | 40.480 | 14.803 | 1.00 | 0.00 | H |
| ATOM | 2616 | N | ARG | A | 333 | 17.779 | 36.259 | 13.261 | 1.00 | 0.00 | N |
| ATOM | 2617 | CA | ARG | A | 333 | 17.801 | 34.931 | 12.684 | 1.00 | 0.00 | C |
| ATOM | 2618 | C | ARG | A | 333 | 19.250 | 34.599 | 12.312 | 1.00 | 0.00 | C |
| ATOM | 2619 | O | ARG | A | 333 | 19.526 | 33.668 | 11.554 | 1.00 | 0.00 | O |
| ATOM | 2620 | CB | ARG | A | 333 | 16.769 | 34.867 | 11.540 | 1.00 | 0.00 | C |
| ATOM | 2621 | CG | ARG | A | 333 | 15.467 | 35.537 | 12.028 | 1.00 | 0.00 | C |
| ATOM | 2622 | CD | ARG | A | 333 | 14.206 | 35.296 | 11.188 | 1.00 | 0.00 | C |
| ATOM | 2623 | NE | ARG | A | 333 | 13.252 | 36.421 | 11.270 | 1.00 | 0.00 | N |
| ATOM | 2624 | CZ | ARG | A | 333 | 12.892 | 37.163 | 12.330 | 1.00 | 0.00 | C |
| ATOM | 2625 | NH1 | ARG | A | 333 | 13.156 | 36.784 | 13.575 | 1.00 | 0.00 | N1+ |
| ATOM | 2626 | NH2 | ARG | A | 333 | 12.281 | 38.321 | 12.139 | 1.00 | 0.00 | N |
| ATOM | 2627 | H | ARG | A | 333 | 18.151 | 36.999 | 12.682 | 1.00 | 0.00 | H |
| ATOM | 2628 | HA | ARG | A | 333 | 17.309 | 34.232 | 13.361 | 1.00 | 0.00 | H |
| ATOM | 2629 | 1HB | ARG | A | 333 | 17.158 | 35.394 | 10.669 | 1.00 | 0.00 | H |
| ATOM | 2630 | 2HB | ARG | A | 333 | 16.579 | 33.826 | 11.279 | 1.00 | 0.00 | H |
| ATOM | 2631 | 1HG | ARG | A | 333 | 15.226 | 35.179 | 13.029 | 1.00 | 0.00 | H |
| ATOM | 2632 | 2HG | ARG | A | 333 | 15.602 | 36.619 | 12.052 | 1.00 | 0.00 | H |
| ATOM | 2633 | 1HD | ARG | A | 333 | 14.485 | 35.167 | 10.142 | 1.00 | 0.00 | H |
| ATOM | 2634 | 2HD | ARG | A | 333 | 13.699 | 34.398 | 11.541 | 1.00 | 0.00 | H |
| ATOM | 2635 | HE | ARG | A | 333 | 12.848 | 36.607 | 10.363 | 1.00 | 0.00 | H |
| ATOM | 2636 | 1HH1 | ARG | A | 333 | 12.872 | 37.365 | 14.351 | 1.00 | 0.00 | H |
| ATOM | 2637 | 2HH1 | ARG | A | 333 | 13.642 | 35.915 | 13.747 | 1.00 | 0.00 | H |
| ATOM | 2638 | 1HH2 | ARG | A | 333 | 12.008 | 38.884 | 12.932 | 1.00 | 0.00 | H |
| ATOM | 2639 | 2HH2 | ARG | A | 333 | 12.089 | 38.642 | 11.201 | 1.00 | 0.00 | H |
| ATOM | 2640 | N | GLY | A | 334 | 20.185 | 35.356 | 12.894 | 1.00 | 0.00 | N |
| ATOM | 2641 | CA | GLY | A | 334 | 21.579 | 34.990 | 13.026 | 1.00 | 0.00 | C |
| ATOM | 2642 | C | GLY | A | 334 | 22.355 | 35.110 | 11.719 | 1.00 | 0.00 | C |
| ATOM | 2643 | O | GLY | A | 334 | 23.431 | 34.528 | 11.602 | 1.00 | 0.00 | O |
| ATOM | 2644 | H | GLY | A | 334 | 19.938 | 36.255 | 13.283 | 1.00 | 0.00 | H |
| ATOM | 2645 | 1HA | GLY | A | 334 | 22.059 | 35.642 | 13.756 | 1.00 | 0.00 | H |
| ATOM | 2646 | 2HA | GLY | A | 334 | 21.654 | 33.955 | 13.360 | 1.00 | 0.00 | H |
| ATOM | 2647 | N | LYS | A | 335 | 21.843 | 35.858 | 10.742 | 1.00 | 0.00 | N |
| ATOM | 2648 | CA | LYS | A | 335 | 22.464 | 35.978 | 9.430 | 1.00 | 0.00 | C |
| ATOM | 2649 | C | LYS | A | 335 | 23.287 | 37.261 | 9.335 | 1.00 | 0.00 | C |
| ATOM | 2650 | O | LYS | A | 335 | 24.110 | 37.399 | 8.433 | 1.00 | 0.00 | O |
| ATOM | 2651 | CB | LYS | A | 335 | 21.389 | 35.962 | 8.335 | 1.00 | 0.00 | C |
| ATOM | 2652 | CG | LYS | A | 335 | 20.351 | 34.843 | 8.494 | 1.00 | 0.00 | C |
| ATOM | 2653 | CD | LYS | A | 335 | 21.019 | 33.466 | 8.593 | 1.00 | 0.00 | C |
| ATOM | 2654 | CE | LYS | A | 335 | 19.979 | 32.341 | 8.601 | 1.00 | 0.00 | C |
| ATOM | 2655 | NZ | LYS | A | 335 | 20.613 | 31.046 | 8.902 | 1.00 | 0.00 | N1+ |
| ATOM | 2656 | H | LYS | A | 335 | 20.988 | 36.373 | 10.895 | 1.00 | 0.00 | H |
| ATOM | 2657 | HA | LYS | A | 335 | 23.096 | 35.109 | 9.246 | 1.00 | 0.00 | H |
| ATOM | 2658 | 1HB | LYS | A | 335 | 20.846 | 36.907 | 8.345 | 1.00 | 0.00 | H |
| ATOM | 2659 | 2HB | LYS | A | 335 | 21.862 | 35.825 | 7.363 | 1.00 | 0.00 | H |
| ATOM | 2660 | 1HG | LYS | A | 335 | 19.771 | 35.010 | 9.401 | 1.00 | 0.00 | H |
| ATOM | 2661 | 2HG | LYS | A | 335 | 19.683 | 34.841 | 7.632 | 1.00 | 0.00 | H |
| ATOM | 2662 | 1HD | LYS | A | 335 | 21.680 | 33.321 | 7.738 | 1.00 | 0.00 | H |
| ATOM | 2663 | 2HD | LYS | A | 335 | 21.599 | 33.409 | 9.514 | 1.00 | 0.00 | H |
| ATOM | 2664 | 1HE | LYS | A | 335 | 19.225 | 32.548 | 9.361 | 1.00 | 0.00 | H |
| ATOM | 2665 | 2HE | LYS | A | 335 | 19.502 | 32.280 | 7.623 | 1.00 | 0.00 | H |
| ATOM | 2666 | 1HZ | LYS | A | 335 | 19.911 | 30.319 | 8.903 | 1.00 | 0.00 | H |
| ATOM | 2667 | 2HZ | LYS | A | 335 | 21.309 | 30.840 | 8.200 | 1.00 | 0.00 | H |
| ATOM | 2668 | 3HZ | LYS | A | 335 | 21.053 | 31.088 | 9.810 | 1.00 | 0.00 | H |
| ATOM | 2669 | N | ILE | A | 336 | 23.057 | 38.205 | 10.244 | 1.00 | 0.00 | N |
| ATOM | 2670 | CA | ILE | A | 336 | 23.633 | 39.538 | 10.190 | 1.00 | 0.00 | C |
| ATOM | 2671 | C | ILE | A | 336 | 24.018 | 39.940 | 11.605 | 1.00 | 0.00 | C |
| ATOM | 2672 | O | ILE | A | 336 | 23.314 | 39.621 | 12.558 | 1.00 | 0.00 | O |
| ATOM | 2673 | CB | ILE | A | 336 | 22.610 | 40.534 | 9.610 | 1.00 | 0.00 | C |
| ATOM | 2674 | CG1 | ILE | A | 336 | 22.322 | 40.285 | 8.120 | 1.00 | 0.00 | C |
| ATOM | 2675 | CG2 | ILE | A | 336 | 23.039 | 41.995 | 9.828 | 1.00 | 0.00 | C |
| ATOM | 2676 | CD1 | ILE | A | 336 | 23.510 | 40.535 | 7.193 | 1.00 | 0.00 | C |
| ATOM | 2677 | H | ILE | A | 336 | 22.451 | 38.013 | 11.029 | 1.00 | 0.00 | H |
| ATOM | 2678 | HA | ILE | A | 336 | 24.508 | 39.531 | 9.540 | 1.00 | 0.00 | H |
| ATOM | 2679 | HB | ILE | A | 336 | 21.698 | 40.506 | 10.205 | 1.00 | 0.00 | H |
| ATOM | 2680 | 1HG1 | ILE | A | 336 | 22.021 | 39.247 | 7.976 | 1.00 | 0.00 | H |
| ATOM | 2681 | 2HG1 | ILE | A | 336 | 21.519 | 40.944 | 7.790 | 1.00 | 0.00 | H |
| ATOM | 2682 | 1HG2 | ILE | A | 336 | 22.288 | 42.662 | 9.404 | 1.00 | 0.00 | H |
| ATOM | 2683 | 2HG2 | ILE | A | 336 | 23.134 | 42.190 | 10.897 | 1.00 | 0.00 | H |
| ATOM | 2684 | 3HG2 | ILE | A | 336 | 23.997 | 42.170 | 9.340 | 1.00 | 0.00 | H |
| ATOM | 2685 | 1HD1 | ILE | A | 336 | 23.217 | 40.335 | 6.163 | 1.00 | 0.00 | H |
| ATOM | 2686 | 2HD1 | ILE | A | 336 | 23.830 | 41.573 | 7.285 | 1.00 | 0.00 | H |
| ATOM | 2687 | 3HD1 | ILE | A | 336 | 24.333 | 39.876 | 7.471 | 1.00 | 0.00 | H |
| ATOM | 2688 | N | GLY | A | 337 | 25.133 | 40.644 | 11.727 | 1.00 | 0.00 | N |
| ATOM | 2689 | CA | GLY | A | 337 | 25.565 | 41.309 | 12.934 | 1.00 | 0.00 | C |
| ATOM | 2690 | C | GLY | A | 337 | 26.341 | 42.534 | 12.477 | 1.00 | 0.00 | C |
| ATOM | 2691 | O | GLY | A | 337 | 26.918 | 42.512 | 11.390 | 1.00 | 0.00 | O |
| ATOM | 2692 | H | GLY | A | 337 | 25.755 | 40.746 | 10.938 | 1.00 | 0.00 | H |
| ATOM | 2693 | 1HA | GLY | A | 337 | 24.694 | 41.592 | 13.526 | 1.00 | 0.00 | H |
| ATOM | 2694 | 2HA | GLY | A | 337 | 26.195 | 40.636 | 13.515 | 1.00 | 0.00 | H |
| ATOM | 2695 | N | LEU | A | 338 | 26.313 | 43.619 | 13.239 | 1.00 | 0.00 | N |
| ATOM | 2696 | CA | LEU | A | 338 | 26.924 | 44.883 | 12.862 | 1.00 | 0.00 | C |
| ATOM | 2697 | C | LEU | A | 338 | 27.979 | 45.218 | 13.922 | 1.00 | 0.00 | C |
| ATOM | 2698 | O | LEU | A | 338 | 28.472 | 46.342 | 14.017 | 1.00 | 0.00 | O |
| ATOM | 2699 | CB | LEU | A | 338 | 25.818 | 45.939 | 12.702 | 1.00 | 0.00 | C |
| ATOM | 2700 | CG | LEU | A | 338 | 24.678 | 45.451 | 11.781 | 1.00 | 0.00 | C |
| ATOM | 2701 | CD1 | LEU | A | 338 | 23.457 | 46.360 | 11.883 | 1.00 | 0.00 | C |
| ATOM | 2702 | CD2 | LEU | A | 338 | 25.132 | 45.359 | 10.320 | 1.00 | 0.00 | C |
| ATOM | 2703 | H | LEU | A | 338 | 25.848 | 43.595 | 14.135 | 1.00 | 0.00 | H |
| ATOM | 2704 | HA | LEU | A | 338 | 27.316 | 44.810 | 11.848 | 1.00 | 0.00 | H |
| ATOM | 2705 | 1HB | LEU | A | 338 | 25.390 | 46.168 | 13.678 | 1.00 | 0.00 | H |
| ATOM | 2706 | 2HB | LEU | A | 338 | 26.240 | 46.846 | 12.269 | 1.00 | 0.00 | H |
| ATOM | 2707 | HG | LEU | A | 338 | 24.419 | 44.423 | 12.036 | 1.00 | 0.00 | H |
| ATOM | 2708 | 1HD1 | LEU | A | 338 | 22.672 | 45.991 | 11.223 | 1.00 | 0.00 | H |
| ATOM | 2709 | 2HD1 | LEU | A | 338 | 23.093 | 46.367 | 12.910 | 1.00 | 0.00 | H |
| ATOM | 2710 | 3HD1 | LEU | A | 338 | 23.732 | 47.373 | 11.588 | 1.00 | 0.00 | H |
| ATOM | 2711 | 1HD2 | LEU | A | 338 | 24.304 | 45.013 | 9.702 | 1.00 | 0.00 | H |
| ATOM | 2712 | 2HD2 | LEU | A | 338 | 25.454 | 46.343 | 9.977 | 1.00 | 0.00 | H |
| ATOM | 2713 | 3HD2 | LEU | A | 338 | 25.963 | 44.658 | 10.240 | 1.00 | 0.00 | H |
| ATOM | 2714 | N | ASP | A | 339 | 28.348 | 44.181 | 14.681 | 1.00 | 0.00 | N |
| ATOM | 2715 | CA | ASP | A | 339 | 29.134 | 44.181 | 15.900 | 1.00 | 0.00 | C |
| ATOM | 2716 | C | ASP | A | 339 | 30.447 | 44.930 | 15.679 | 1.00 | 0.00 | C |
| ATOM | 2717 | O | ASP | A | 339 | 31.018 | 45.515 | 16.596 | 1.00 | 0.00 | O |
| ATOM | 2718 | CB | ASP | A | 339 | 29.473 | 42.726 | 16.294 | 1.00 | 0.00 | C |
| ATOM | 2719 | CG | ASP | A | 339 | 28.328 | 41.725 | 16.238 | 1.00 | 0.00 | C |
| ATOM | 2720 | OD1 | ASP | A | 339 | 28.138 | 40.958 | 17.206 | 1.00 | 0.00 | O |
| ATOM | 2721 | OD2 | ASP | A | 339 | 27.684 | 41.582 | 15.174 | 1.00 | 0.00 | O1− |
| ATOM | 2722 | H | ASP | A | 339 | 28.073 | 43.244 | 14.423 | 1.00 | 0.00 | H |
| ATOM | 2723 | HA | ASP | A | 339 | 28.582 | 44.691 | 16.689 | 1.00 | 0.00 | H |
| ATOM | 2724 | 1HB | ASP | A | 339 | 30.242 | 42.337 | 15.625 | 1.00 | 0.00 | H |
| ATOM | 2725 | 2HB | ASP | A | 339 | 29.840 | 42.703 | 17.320 | 1.00 | 0.00 | H |
| ATOM | 2726 | N | PHE | A | 340 | 30.971 | 44.822 | 14.456 | 1.00 | 0.00 | N |
| ATOM | 2727 | CA | PHE | A | 340 | 32.280 | 45.307 | 14.063 | 1.00 | 0.00 | C |
| ATOM | 2728 | C | PHE | A | 340 | 32.145 | 46.039 | 12.723 | 1.00 | 0.00 | C |
| ATOM | 2729 | O | PHE | A | 340 | 33.005 | 45.912 | 11.846 | 1.00 | 0.00 | O |
| ATOM | 2730 | CB | PHE | A | 340 | 33.253 | 44.115 | 13.954 | 1.00 | 0.00 | C |
| ATOM | 2731 | CG | PHE | A | 340 | 33.173 | 43.082 | 15.066 | 1.00 | 0.00 | C |
| ATOM | 2732 | CD1 | PHE | A | 340 | 33.279 | 43.462 | 16.418 | 1.00 | 0.00 | C |
| ATOM | 2733 | CD2 | PHE | A | 340 | 32.939 | 41.725 | 14.763 | 1.00 | 0.00 | C |
| ATOM | 2734 | CE1 | PHE | A | 340 | 33.072 | 42.525 | 17.445 | 1.00 | 0.00 | C |
| ATOM | 2735 | CE2 | PHE | A | 340 | 32.741 | 40.783 | 15.786 | 1.00 | 0.00 | C |
| ATOM | 2736 | CZ | PHE | A | 340 | 32.786 | 41.186 | 17.130 | 1.00 | 0.00 | C |
| ATOM | 2737 | H | PHE | A | 340 | 30.446 | 44.373 | 13.719 | 1.00 | 0.00 | H |
| ATOM | 2738 | HA | PHE | A | 340 | 32.670 | 45.969 | 14.836 | 1.00 | 0.00 | H |
| ATOM | 2739 | 1HB | PHE | A | 340 | 33.064 | 43.575 | 13.026 | 1.00 | 0.00 | H |
| ATOM | 2740 | 2HB | PHE | A | 340 | 34.280 | 44.482 | 13.957 | 1.00 | 0.00 | H |
| ATOM | 2741 | HD1 | PHE | A | 340 | 33.522 | 44.483 | 16.674 | 1.00 | 0.00 | H |
| ATOM | 2742 | HD2 | PHE | A | 340 | 32.911 | 41.398 | 13.734 | 1.00 | 0.00 | H |
| ATOM | 2743 | HE1 | PHE | A | 340 | 33.132 | 42.833 | 18.478 | 1.00 | 0.00 | H |
| ATOM | 2744 | HE2 | PHE | A | 340 | 32.553 | 39.748 | 15.538 | 1.00 | 0.00 | H |
| ATOM | 2745 | HZ | PHE | A | 340 | 32.602 | 40.473 | 17.920 | 1.00 | 0.00 | H |
| ATOM | 2746 | N | CYS | A | 341 | 31.041 | 46.761 | 12.536 | 1.00 | 0.00 | N |
| ATOM | 2747 | CA | CYS | A | 341 | 30.733 | 47.507 | 11.320 | 1.00 | 0.00 | C |
| ATOM | 2748 | C | CYS | A | 341 | 31.140 | 48.962 | 11.550 | 1.00 | 0.00 | C |
| ATOM | 2749 | O | CYS | A | 341 | 30.590 | 49.619 | 12.427 | 1.00 | 0.00 | O |
| ATOM | 2750 | CB | CYS | A | 341 | 29.230 | 47.375 | 11.034 | 1.00 | 0.00 | C |
| ATOM | 2751 | SG | CYS | A | 341 | 28.784 | 48.121 | 9.445 | 1.00 | 0.00 | S |
| ATOM | 2752 | H | CYS | A | 341 | 30.346 | 46.820 | 13.266 | 1.00 | 0.00 | H |
| ATOM | 2753 | HA | CYS | A | 341 | 31.274 | 47.073 | 10.480 | 1.00 | 0.00 | H |
| ATOM | 2754 | 1HB | CYS | A | 341 | 28.956 | 46.321 | 11.008 | 1.00 | 0.00 | H |
| ATOM | 2755 | 2HB | CYS | A | 341 | 28.665 | 47.877 | 11.820 | 1.00 | 0.00 | H |
| ATOM | 2756 | HG | CYS | A | 341 | 27.703 | 48.007 | 9.276 | 1.00 | 0.00 | H |
| ATOM | 2757 | N | LYS | A | 342 | 32.111 | 49.476 | 10.791 | 1.00 | 0.00 | N |
| ATOM | 2758 | CA | LYS | A | 342 | 32.703 | 50.777 | 11.085 | 1.00 | 0.00 | C |
| ATOM | 2759 | C | LYS | A | 342 | 31.913 | 51.913 | 10.437 | 1.00 | 0.00 | C |
| ATOM | 2760 | O | LYS | A | 342 | 32.294 | 53.069 | 10.617 | 1.00 | 0.00 | O |
| ATOM | 2761 | CB | LYS | A | 342 | 34.160 | 50.843 | 10.583 | 1.00 | 0.00 | C |
| ATOM | 2762 | CG | LYS | A | 342 | 35.251 | 50.561 | 11.621 | 1.00 | 0.00 | C |
| ATOM | 2763 | CD | LYS | A | 342 | 35.400 | 51.613 | 12.740 | 1.00 | 0.00 | C |
| ATOM | 2764 | CE | LYS | A | 342 | 35.574 | 53.081 | 12.285 | 1.00 | 0.00 | C |
| ATOM | 2765 | NZ | LYS | A | 342 | 34.307 | 53.852 | 12.236 | 1.00 | 0.00 | N1+ |
| ATOM | 2766 | H | LYS | A | 342 | 32.453 | 48.962 | 9.992 | 1.00 | 0.00 | H |
| ATOM | 2767 | HA | LYS | A | 342 | 32.730 | 50.928 | 12.164 | 1.00 | 0.00 | H |
| ATOM | 2768 | 1HB | LYS | A | 342 | 34.305 | 50.108 | 9.791 | 1.00 | 0.00 | H |
| ATOM | 2769 | 2HB | LYS | A | 342 | 34.367 | 51.840 | 10.196 | 1.00 | 0.00 | H |
| ATOM | 2770 | 1HG | LYS | A | 342 | 35.045 | 49.614 | 12.119 | 1.00 | 0.00 | H |
| ATOM | 2771 | 2HG | LYS | A | 342 | 36.220 | 50.506 | 11.125 | 1.00 | 0.00 | H |
| ATOM | 2772 | 1HD | LYS | A | 342 | 34.512 | 51.599 | 13.372 | 1.00 | 0.00 | H |
| ATOM | 2773 | 2HD | LYS | A | 342 | 36.278 | 51.381 | 13.343 | 1.00 | 0.00 | H |
| ATOM | 2774 | 1HE | LYS | A | 342 | 36.239 | 53.600 | 12.975 | 1.00 | 0.00 | H |
| ATOM | 2775 | 2HE | LYS | A | 342 | 36.003 | 53.103 | 11.283 | 1.00 | 0.00 | H |
| ATOM | 2776 | 1HZ | LYS | A | 342 | 34.498 | 54.796 | 11.932 | 1.00 | 0.00 | H |
| ATOM | 2777 | 2HZ | LYS | A | 342 | 33.670 | 53.413 | 11.587 | 1.00 | 0.00 | H |
| ATOM | 2778 | 3HZ | LYS | A | 342 | 33.889 | 53.873 | 13.155 | 1.00 | 0.00 | H |
| ATOM | 2779 | N | TYR | A | 343 | 30.891 | 51.620 | 9.636 | 1.00 | 0.00 | N |
| ATOM | 2780 | CA | TYR | A | 343 | 30.311 | 52.594 | 8.722 | 1.00 | 0.00 | C |
| ATOM | 2781 | C | TYR | A | 343 | 28.823 | 52.302 | 8.544 | 1.00 | 0.00 | C |
| ATOM | 2782 | O | TYR | A | 343 | 28.446 | 51.144 | 8.356 | 1.00 | 0.00 | O |
| ATOM | 2783 | CB | TYR | A | 343 | 31.050 | 52.517 | 7.377 | 1.00 | 0.00 | C |
| ATOM | 2784 | CG | TYR | A | 343 | 32.508 | 52.914 | 7.469 | 1.00 | 0.00 | C |
| ATOM | 2785 | CD1 | TYR | A | 343 | 32.853 | 54.264 | 7.636 | 1.00 | 0.00 | C |
| ATOM | 2786 | CD2 | TYR | A | 343 | 33.533 | 51.950 | 7.456 | 1.00 | 0.00 | C |
| ATOM | 2787 | CE1 | TYR | A | 343 | 34.177 | 54.636 | 7.918 | 1.00 | 0.00 | C |
| ATOM | 2788 | CE2 | TYR | A | 343 | 34.863 | 52.322 | 7.716 | 1.00 | 0.00 | C |
| ATOM | 2789 | CZ | TYR | A | 343 | 35.181 | 53.661 | 8.009 | 1.00 | 0.00 | C |
| ATOM | 2790 | OH | TYR | A | 343 | 36.444 | 54.011 | 8.390 | 1.00 | 0.00 | O |
| ATOM | 2791 | H | TYR | A | 343 | 30.490 | 50.693 | 9.648 | 1.00 | 0.00 | H |
| ATOM | 2792 | HA | TYR | A | 343 | 30.461 | 53.599 | 9.118 | 1.00 | 0.00 | H |
| ATOM | 2793 | 1HB | TYR | A | 343 | 31.013 | 51.494 | 7.000 | 1.00 | 0.00 | H |
| ATOM | 2794 | 2HB | TYR | A | 343 | 30.574 | 53.185 | 6.660 | 1.00 | 0.00 | H |
| ATOM | 2795 | HD1 | TYR | A | 343 | 32.096 | 55.030 | 7.547 | 1.00 | 0.00 | H |
| ATOM | 2796 | HD2 | TYR | A | 343 | 33.299 | 50.916 | 7.245 | 1.00 | 0.00 | H |
| ATOM | 2797 | HE1 | TYR | A | 343 | 34.425 | 55.677 | 8.067 | 1.00 | 0.00 | H |
| ATOM | 2798 | HE2 | TYR | A | 343 | 35.646 | 51.579 | 7.693 | 1.00 | 0.00 | H |
| ATOM | 2799 | HH | TYR | A | 343 | 37.004 | 53.231 | 8.400 | 1.00 | 0.00 | H |
| ATOM | 2800 | N | LEU | A | 344 | 27.995 | 53.343 | 8.606 | 1.00 | 0.00 | N |
| ATOM | 2801 | CA | LEU | A | 344 | 26.551 | 53.265 | 8.476 | 1.00 | 0.00 | C |
| ATOM | 2802 | C | LEU | A | 344 | 26.098 | 54.428 | 7.598 | 1.00 | 0.00 | C |
| ATOM | 2803 | O | LEU | A | 344 | 26.349 | 55.582 | 7.942 | 1.00 | 0.00 | O |
| ATOM | 2804 | CB | LEU | A | 344 | 25.914 | 53.364 | 9.868 | 1.00 | 0.00 | C |
| ATOM | 2805 | CG | LEU | A | 344 | 24.396 | 53.616 | 9.825 | 1.00 | 0.00 | C |
| ATOM | 2806 | CD1 | LEU | A | 344 | 23.631 | 52.494 | 9.113 | 1.00 | 0.00 | C |
| ATOM | 2807 | CD2 | LEU | A | 344 | 23.877 | 53.746 | 11.256 | 1.00 | 0.00 | C |
| ATOM | 2808 | H | LEU | A | 344 | 28.362 | 54.273 | 8.753 | 1.00 | 0.00 | H |
| ATOM | 2809 | HA | LEU | A | 344 | 26.278 | 52.315 | 8.015 | 1.00 | 0.00 | H |
| ATOM | 2810 | 1HB | LEU | A | 344 | 26.077 | 52.432 | 10.410 | 1.00 | 0.00 | H |
| ATOM | 2811 | 2HB | LEU | A | 344 | 26.368 | 54.188 | 10.418 | 1.00 | 0.00 | H |
| ATOM | 2812 | HG | LEU | A | 344 | 24.198 | 54.557 | 9.312 | 1.00 | 0.00 | H |
| ATOM | 2813 | 1HD1 | LEU | A | 344 | 22.565 | 52.724 | 9.112 | 1.00 | 0.00 | H |
| ATOM | 2814 | 2HD1 | LEU | A | 344 | 23.984 | 52.408 | 8.085 | 1.00 | 0.00 | H |
| ATOM | 2815 | 3HD1 | LEU | A | 344 | 23.798 | 51.551 | 9.634 | 1.00 | 0.00 | H |
| ATOM | 2816 | 1HD2 | LEU | A | 344 | 22.802 | 53.924 | 11.238 | 1.00 | 0.00 | H |
| ATOM | 2817 | 2HD2 | LEU | A | 344 | 24.083 | 52.825 | 11.802 | 1.00 | 0.00 | H |
| ATOM | 2818 | 3HD2 | LEU | A | 344 | 24.376 | 54.580 | 11.749 | 1.00 | 0.00 | H |
| ATOM | 2819 | N | VAL | A | 345 | 25.407 | 54.126 | 6.501 | 1.00 | 0.00 | N |
| ATOM | 2820 | CA | VAL | A | 345 | 24.861 | 55.116 | 5.587 | 1.00 | 0.00 | C |
| ATOM | 2821 | C | VAL | A | 345 | 23.342 | 54.963 | 5.544 | 1.00 | 0.00 | C |
| ATOM | 2822 | O | VAL | A | 345 | 22.829 | 53.847 | 5.632 | 1.00 | 0.00 | O |
| ATOM | 2823 | CB | VAL | A | 345 | 25.437 | 54.907 | 4.179 | 1.00 | 0.00 | C |
| ATOM | 2824 | CG1 | VAL | A | 345 | 24.920 | 55.965 | 3.195 | 1.00 | 0.00 | C |
| ATOM | 2825 | CG2 | VAL | A | 345 | 26.965 | 54.936 | 4.157 | 1.00 | 0.00 | C |
| ATOM | 2826 | H | VAL | A | 345 | 25.234 | 53.159 | 6.263 | 1.00 | 0.00 | H |
| ATOM | 2827 | HA | VAL | A | 345 | 25.122 | 56.115 | 5.935 | 1.00 | 0.00 | H |
| ATOM | 2828 | HB | VAL | A | 345 | 25.180 | 53.909 | 3.825 | 1.00 | 0.00 | H |
| ATOM | 2829 | 1HG1 | VAL | A | 345 | 25.350 | 55.785 | 2.209 | 1.00 | 0.00 | H |
| ATOM | 2830 | 2HG1 | VAL | A | 345 | 23.834 | 55.905 | 3.134 | 1.00 | 0.00 | H |
| ATOM | 2831 | 3HG1 | VAL | A | 345 | 25.210 | 56.957 | 3.541 | 1.00 | 0.00 | H |
| ATOM | 2832 | 1HG2 | VAL | A | 345 | 27.316 | 54.784 | 3.136 | 1.00 | 0.00 | H |
| ATOM | 2833 | 2HG2 | VAL | A | 345 | 27.316 | 55.902 | 4.521 | 1.00 | 0.00 | H |
| ATOM | 2834 | 3HG2 | VAL | A | 345 | 27.353 | 54.144 | 4.796 | 1.00 | 0.00 | H |
| ATOM | 2835 | N | LEU | A | 346 | 22.631 | 56.066 | 5.332 | 1.00 | 0.00 | N |
| ATOM | 2836 | CA | LEU | A | 346 | 21.218 | 56.077 | 5.004 | 1.00 | 0.00 | C |
| ATOM | 2837 | C | LEU | A | 346 | 21.062 | 57.021 | 3.805 | 1.00 | 0.00 | C |
| ATOM | 2838 | O | LEU | A | 346 | 21.665 | 58.091 | 3.842 | 1.00 | 0.00 | O |
| ATOM | 2839 | CB | LEU | A | 346 | 20.399 | 56.565 | 6.212 | 1.00 | 0.00 | C |
| ATOM | 2840 | CG | LEU | A | 346 | 20.653 | 55.808 | 7.532 | 1.00 | 0.00 | C |
| ATOM | 2841 | CD1 | LEU | A | 346 | 21.776 | 56.432 | 8.375 | 1.00 | 0.00 | C |
| ATOM | 2842 | CD2 | LEU | A | 346 | 19.377 | 55.815 | 8.385 | 1.00 | 0.00 | C |
| ATOM | 2843 | H | LEU | A | 346 | 23.073 | 56.972 | 5.395 | 1.00 | 0.00 | H |
| ATOM | 2844 | HA | LEU | A | 346 | 20.892 | 55.065 | 4.762 | 1.00 | 0.00 | H |
| ATOM | 2845 | 1HB | LEU | A | 346 | 20.629 | 57.613 | 6.408 | 1.00 | 0.00 | H |
| ATOM | 2846 | 2HB | LEU | A | 346 | 19.336 | 56.462 | 5.997 | 1.00 | 0.00 | H |
| ATOM | 2847 | HG | LEU | A | 346 | 20.939 | 54.779 | 7.312 | 1.00 | 0.00 | H |
| ATOM | 2848 | 1HD1 | LEU | A | 346 | 21.908 | 55.856 | 9.290 | 1.00 | 0.00 | H |
| ATOM | 2849 | 2HD1 | LEU | A | 346 | 22.705 | 56.426 | 7.805 | 1.00 | 0.00 | H |
| ATOM | 2850 | 3HD1 | LEU | A | 346 | 21.513 | 57.459 | 8.628 | 1.00 | 0.00 | H |
| ATOM | 2851 | 1HD2 | LEU | A | 346 | 19.559 | 55.279 | 9.317 | 1.00 | 0.00 | H |
| ATOM | 2852 | 2HD2 | LEU | A | 346 | 19.094 | 56.844 | 8.607 | 1.00 | 0.00 | H |
| ATOM | 2853 | 3HD2 | LEU | A | 346 | 18.571 | 55.327 | 7.837 | 1.00 | 0.00 | H |
| ATOM | 2854 | N | ASP | A | 347 | 20.302 | 56.651 | 2.764 | 1.00 | 0.00 | N |
| ATOM | 2855 | CA | ASP | A | 347 | 20.027 | 57.511 | 1.603 | 1.00 | 0.00 | C |
| ATOM | 2856 | C | ASP | A | 347 | 18.540 | 57.443 | 1.239 | 1.00 | 0.00 | C |
| ATOM | 2857 | O | ASP | A | 347 | 17.883 | 56.435 | 1.512 | 1.00 | 0.00 | O |
| ATOM | 2858 | CB | ASP | A | 347 | 20.924 | 57.169 | 0.397 | 1.00 | 0.00 | C |
| ATOM | 2859 | CG | ASP | A | 347 | 20.761 | 58.157 | −0.757 | 1.00 | 0.00 | C |
| ATOM | 2860 | OD1 | ASP | A | 347 | 20.304 | 59.294 | −0.522 | 1.00 | 0.00 | O |
| ATOM | 2861 | OD2 | ASP | A | 347 | 21.081 | 57.807 | −1.914 | 1.00 | 0.00 | O1− |
| ATOM | 2862 | H | ASP | A | 347 | 19.880 | 55.733 | 2.748 | 1.00 | 0.00 | H |
| ATOM | 2863 | HA | ASP | A | 347 | 20.371 | 58.524 | 1.812 | 1.00 | 0.00 | H |
| ATOM | 2864 | 1HB | ASP | A | 347 | 21.970 | 57.185 | 0.704 | 1.00 | 0.00 | H |
| ATOM | 2865 | 2HB | ASP | A | 347 | 20.670 | 56.176 | 0.025 | 1.00 | 0.00 | H |
| ATOM | 2866 | N | GLU | A | 348 | 18.029 | 58.539 | 0.665 | 1.00 | 0.00 | N |
| ATOM | 2867 | CA | GLU | A | 348 | 16.677 | 59.072 | 0.814 | 1.00 | 0.00 | C |
| ATOM | 2868 | C | GLU | A | 348 | 16.060 | 58.569 | 2.125 | 1.00 | 0.00 | C |
| ATOM | 2869 | O | GLU | A | 348 | 15.032 | 57.891 | 2.151 | 1.00 | 0.00 | O |
| ATOM | 2870 | CB | GLU | A | 348 | 15.835 | 58.944 | −0.478 | 1.00 | 0.00 | C |
| ATOM | 2871 | CG | GLU | A | 348 | 16.692 | 59.452 | −1.655 | 1.00 | 0.00 | C |
| ATOM | 2872 | CD | GLU | A | 348 | 16.012 | 59.946 | −2.928 | 1.00 | 0.00 | C |
| ATOM | 2873 | OE1 | GLU | A | 348 | 14.773 | 59.879 | −3.096 | 1.00 | 0.00 | O |
| ATOM | 2874 | OE2 | GLU | A | 348 | 16.816 | 60.418 | −3.766 | 1.00 | 0.00 | O1− |
| ATOM | 2875 | H | GLU | A | 348 | 18.600 | 59.099 | 0.049 | 1.00 | 0.00 | H |
| ATOM | 2876 | HA | GLU | A | 348 | 16.679 | 60.138 | 0.586 | 1.00 | 0.00 | H |
| ATOM | 2877 | 1HB | GLU | A | 348 | 15.562 | 57.901 | −0.633 | 1.00 | 0.00 | H |
| ATOM | 2878 | 2HB | GLU | A | 348 | 14.930 | 59.546 | −0.383 | 1.00 | 0.00 | H |
| ATOM | 2879 | 1HG | GLU | A | 348 | 17.293 | 60.301 | −1.328 | 1.00 | 0.00 | H |
| ATOM | 2880 | 2HG | GLU | A | 348 | 17.349 | 58.653 | −1.997 | 1.00 | 0.00 | H |
| ATOM | 2881 | N | ALA | A | 349 | 16.772 | 58.870 | 3.219 | 1.00 | 0.00 | N |
| ATOM | 2882 | CA | ALA | A | 349 | 16.468 | 58.374 | 4.548 | 1.00 | 0.00 | C |
| ATOM | 2883 | C | ALA | A | 349 | 15.187 | 59.003 | 5.076 | 1.00 | 0.00 | C |
| ATOM | 2884 | O | ALA | A | 349 | 14.519 | 58.439 | 5.938 | 1.00 | 0.00 | O |
| ATOM | 2885 | CB | ALA | A | 349 | 17.637 | 58.696 | 5.479 | 1.00 | 0.00 | C |
| ATOM | 2886 | H | ALA | A | 349 | 17.576 | 59.478 | 3.147 | 1.00 | 0.00 | H |
| ATOM | 2887 | HA | ALA | A | 349 | 16.363 | 57.289 | 4.516 | 1.00 | 0.00 | H |
| ATOM | 2888 | 1HB | ALA | A | 349 | 17.416 | 58.327 | 6.481 | 1.00 | 0.00 | H |
| ATOM | 2889 | 2HB | ALA | A | 349 | 18.542 | 58.217 | 5.106 | 1.00 | 0.00 | H |
| ATOM | 2890 | 3HB | ALA | A | 349 | 17.786 | 59.775 | 5.515 | 1.00 | 0.00 | H |
| ATOM | 2891 | N | ASP | A | 350 | 14.893 | 60.189 | 4.559 | 1.00 | 0.00 | N |
| ATOM | 2892 | CA | ASP | A | 350 | 13.606 | 60.847 | 4.619 | 1.00 | 0.00 | C |
| ATOM | 2893 | C | ASP | A | 350 | 12.462 | 59.837 | 4.538 | 1.00 | 0.00 | C |
| ATOM | 2894 | O | ASP | A | 350 | 11.695 | 59.715 | 5.488 | 1.00 | 0.00 | O |
| ATOM | 2895 | CB | ASP | A | 350 | 13.549 | 61.908 | 3.520 | 1.00 | 0.00 | C |
| ATOM | 2896 | CG | ASP | A | 350 | 14.101 | 61.424 | 2.192 | 1.00 | 0.00 | C |
| ATOM | 2897 | OD1 | ASP | A | 350 | 13.371 | 60.708 | 1.478 | 1.00 | 0.00 | O |
| ATOM | 2898 | OD2 | ASP | A | 350 | 15.285 | 61.725 | 1.933 | 1.00 | 0.00 | O1− |
| ATOM | 2899 | H | ASP | A | 350 | 15.602 | 60.717 | 4.069 | 1.00 | 0.00 | H |
| ATOM | 2900 | HA | ASP | A | 350 | 13.555 | 61.467 | 5.514 | 1.00 | 0.00 | H |
| ATOM | 2901 | 1HB | ASP | A | 350 | 12.514 | 62.208 | 3.355 | 1.00 | 0.00 | H |
| ATOM | 2902 | 2HB | ASP | A | 350 | 14.135 | 62.776 | 3.822 | 1.00 | 0.00 | H |
| ATOM | 2903 | N | ARG | A | 351 | 12.364 | 59.051 | 3.467 | 1.00 | 0.00 | N |
| ATOM | 2904 | CA | ARG | A | 351 | 11.253 | 58.118 | 3.312 | 1.00 | 0.00 | C |
| ATOM | 2905 | C | ARG | A | 351 | 11.480 | 56.808 | 4.071 | 1.00 | 0.00 | C |
| ATOM | 2906 | O | ARG | A | 351 | 10.882 | 55.783 | 3.748 | 1.00 | 0.00 | O |
| ATOM | 2907 | CB | ARG | A | 351 | 10.881 | 57.968 | 1.831 | 1.00 | 0.00 | C |
| ATOM | 2908 | CG | ARG | A | 351 | 9.653 | 57.104 | 1.499 | 1.00 | 0.00 | C |
| ATOM | 2909 | CD | ARG | A | 351 | 8.834 | 57.700 | 0.337 | 1.00 | 0.00 | C |
| ATOM | 2910 | NE | ARG | A | 351 | 7.925 | 58.767 | 0.804 | 1.00 | 0.00 | N |
| ATOM | 2911 | CZ | ARG | A | 351 | 7.275 | 59.688 | 0.071 | 1.00 | 0.00 | C |
| ATOM | 2912 | NH1 | ARG | A | 351 | 6.411 | 60.496 | 0.663 | 1.00 | 0.00 | N1+ |
| ATOM | 2913 | NH2 | ARG | A | 351 | 7.498 | 59.855 | −1.230 | 1.00 | 0.00 | N |
| ATOM | 2914 | H | ARG | A | 351 | 13.066 | 59.094 | 2.742 | 1.00 | 0.00 | H |
| ATOM | 2915 | HA | ARG | A | 351 | 10.314 | 58.634 | 3.515 | 1.00 | 0.00 | H |
| ATOM | 2916 | 1HB | ARG | A | 351 | 10.668 | 58.949 | 1.407 | 1.00 | 0.00 | H |
| ATOM | 2917 | 2HB | ARG | A | 351 | 11.711 | 57.512 | 1.291 | 1.00 | 0.00 | H |
| ATOM | 2918 | 1HG | ARG | A | 351 | 9.979 | 56.104 | 1.212 | 1.00 | 0.00 | H |
| ATOM | 2919 | 2HG | ARG | A | 351 | 9.008 | 57.038 | 2.375 | 1.00 | 0.00 | H |
| ATOM | 2920 | 1HD | ARG | A | 351 | 9.511 | 58.125 | −0.405 | 1.00 | 0.00 | H |
| ATOM | 2921 | 2HD | ARG | A | 351 | 8.235 | 56.916 | −0.125 | 1.00 | 0.00 | H |
| ATOM | 2922 | HE | ARG | A | 351 | 7.817 | 58.753 | 1.808 | 1.00 | 0.00 | H |
| ATOM | 2923 | 1HH1 | ARG | A | 351 | 5.917 | 61.191 | 0.122 | 1.00 | 0.00 | H |
| ATOM | 2924 | 2HH1 | ARG | A | 351 | 6.244 | 60.417 | 1.656 | 1.00 | 0.00 | H |
| ATOM | 2925 | 1HH2 | ARG | A | 351 | 6.988 | 60.559 | −1.744 | 1.00 | 0.00 | H |
| ATOM | 2926 | 2HH2 | ARG | A | 351 | 8.178 | 59.278 | −1.704 | 1.00 | 0.00 | H |
| ATOM | 2927 | N | MET | A | 352 | 12.295 | 56.841 | 5.127 | 1.00 | 0.00 | N |
| ATOM | 2928 | CA | MET | A | 352 | 12.257 | 55.874 | 6.212 | 1.00 | 0.00 | C |
| ATOM | 2929 | C | MET | A | 352 | 11.695 | 56.549 | 7.468 | 1.00 | 0.00 | C |
| ATOM | 2930 | O | MET | A | 352 | 11.638 | 55.932 | 8.529 | 1.00 | 0.00 | O |
| ATOM | 2931 | CB | MET | A | 352 | 13.658 | 55.294 | 6.452 | 1.00 | 0.00 | C |
| ATOM | 2932 | CG | MET | A | 352 | 14.188 | 54.633 | 5.177 | 1.00 | 0.00 | C |
| ATOM | 2933 | SD | MET | A | 352 | 15.628 | 53.552 | 5.395 | 1.00 | 0.00 | S |
| ATOM | 2934 | CE | MET | A | 352 | 16.958 | 54.776 | 5.396 | 1.00 | 0.00 | C |
| ATOM | 2935 | H | MET | A | 352 | 12.991 | 57.569 | 5.205 | 1.00 | 0.00 | H |
| ATOM | 2936 | HA | MET | A | 352 | 11.666 | 55.010 | 5.909 | 1.00 | 0.00 | H |
| ATOM | 2937 | 1HB | MET | A | 352 | 14.338 | 56.093 | 6.745 | 1.00 | 0.00 | H |
| ATOM | 2938 | 2HB | MET | A | 352 | 13.612 | 54.547 | 7.245 | 1.00 | 0.00 | H |
| ATOM | 2939 | 1HG | MET | A | 352 | 13.407 | 54.016 | 4.735 | 1.00 | 0.00 | H |
| ATOM | 2940 | 2HG | MET | A | 352 | 14.489 | 55.403 | 4.467 | 1.00 | 0.00 | H |
| ATOM | 2941 | 1HE | MET | A | 352 | 17.916 | 54.272 | 5.522 | 1.00 | 0.00 | H |
| ATOM | 2942 | 2HE | MET | A | 352 | 16.954 | 55.318 | 4.451 | 1.00 | 0.00 | H |
| ATOM | 2943 | 3HE | MET | A | 352 | 16.807 | 55.477 | 6.217 | 1.00 | 0.00 | H |
| ATOM | 2944 | N | LEU | A | 353 | 11.277 | 57.808 | 7.342 | 1.00 | 0.00 | N |
| ATOM | 2945 | CA | LEU | A | 353 | 10.646 | 58.622 | 8.365 | 1.00 | 0.00 | C |
| ATOM | 2946 | C | LEU | A | 353 | 9.255 | 59.017 | 7.868 | 1.00 | 0.00 | C |
| ATOM | 2947 | O | LEU | A | 353 | 8.318 | 58.993 | 8.663 | 1.00 | 0.00 | O |
| ATOM | 2948 | CB | LEU | A | 353 | 11.490 | 59.857 | 8.713 | 1.00 | 0.00 | C |
| ATOM | 2949 | CG | LEU | A | 353 | 12.935 | 59.523 | 9.121 | 1.00 | 0.00 | C |
| ATOM | 2950 | CD1 | LEU | A | 353 | 13.710 | 60.825 | 9.340 | 1.00 | 0.00 | C |
| ATOM | 2951 | CD2 | LEU | A | 353 | 13.002 | 58.684 | 10.404 | 1.00 | 0.00 | C |
| ATOM | 2952 | H | LEU | A | 353 | 11.387 | 58.291 | 6.462 | 1.00 | 0.00 | H |
| ATOM | 2953 | HA | LEU | A | 353 | 10.614 | 58.067 | 9.303 | 1.00 | 0.00 | H |
| ATOM | 2954 | 1HB | LEU | A | 353 | 11.542 | 60.517 | 7.847 | 1.00 | 0.00 | H |
| ATOM | 2955 | 2HB | LEU | A | 353 | 11.032 | 60.388 | 9.547 | 1.00 | 0.00 | H |
| ATOM | 2956 | HG | LEU | A | 353 | 13.406 | 58.928 | 8.338 | 1.00 | 0.00 | H |
| ATOM | 2957 | 1HD1 | LEU | A | 353 | 14.735 | 60.595 | 9.630 | 1.00 | 0.00 | H |
| ATOM | 2958 | 2HD1 | LEU | A | 353 | 13.716 | 61.405 | 8.417 | 1.00 | 0.00 | H |
| ATOM | 2959 | 3HD1 | LEU | A | 353 | 13.232 | 61.405 | 10.129 | 1.00 | 0.00 | H |
| ATOM | 2960 | 1HD2 | LEU | A | 353 | 14.043 | 58.476 | 10.648 | 1.00 | 0.00 | H |
| ATOM | 2961 | 2HD2 | LEU | A | 353 | 12.540 | 59.235 | 11.223 | 1.00 | 0.00 | H |
| ATOM | 2962 | 3HD2 | LEU | A | 353 | 12.470 | 57.745 | 10.252 | 1.00 | 0.00 | H |
| ATOM | 2963 | N | ASP | A | 354 | 9.083 | 59.257 | 6.556 | 1.00 | 0.00 | N |
| ATOM | 2964 | CA | ASP | A | 354 | 7.757 | 59.390 | 5.938 | 1.00 | 0.00 | C |
| ATOM | 2965 | C | ASP | A | 354 | 6.879 | 58.188 | 6.320 | 1.00 | 0.00 | C |
| ATOM | 2966 | O | ASP | A | 354 | 5.656 | 58.250 | 6.230 | 1.00 | 0.00 | O |
| ATOM | 2967 | CB | ASP | A | 354 | 7.800 | 59.387 | 4.393 | 1.00 | 0.00 | C |
| ATOM | 2968 | CG | ASP | A | 354 | 8.392 | 60.599 | 3.687 | 1.00 | 0.00 | C |
| ATOM | 2969 | OD1 | ASP | A | 354 | 9.587 | 60.892 | 3.863 | 1.00 | 0.00 | O |
| ATOM | 2970 | OD2 | ASP | A | 354 | 7.704 | 61.134 | 2.795 | 1.00 | 0.00 | O1− |
| ATOM | 2971 | H | ASP | A | 354 | 9.885 | 59.353 | 5.950 | 1.00 | 0.00 | H |
| ATOM | 2972 | HA | ASP | A | 354 | 7.264 | 60.283 | 6.323 | 1.00 | 0.00 | H |
| ATOM | 2973 | 1HB | ASP | A | 354 | 8.395 | 58.541 | 4.047 | 1.00 | 0.00 | H |
| ATOM | 2974 | 2HB | ASP | A | 354 | 6.786 | 59.301 | 4.001 | 1.00 | 0.00 | H |
| ATOM | 2975 | N | MET | A | 355 | 7.512 | 57.051 | 6.628 | 1.00 | 0.00 | N |
| ATOM | 2976 | CA | MET | A | 355 | 6.861 | 55.786 | 6.933 | 1.00 | 0.00 | C |
| ATOM | 2977 | C | MET | A | 355 | 7.316 | 55.281 | 8.308 | 1.00 | 0.00 | C |
| ATOM | 2978 | O | MET | A | 355 | 7.150 | 54.107 | 8.627 | 1.00 | 0.00 | O |
| ATOM | 2979 | CB | MET | A | 355 | 7.191 | 54.774 | 5.827 | 1.00 | 0.00 | C |
| ATOM | 2980 | CG | MET | A | 355 | 6.792 | 55.283 | 4.435 | 1.00 | 0.00 | C |
| ATOM | 2981 | SD | MET | A | 355 | 7.130 | 54.142 | 3.065 | 1.00 | 0.00 | S |
| ATOM | 2982 | CE | MET | A | 355 | 5.912 | 52.842 | 3.398 | 1.00 | 0.00 | C |
| ATOM | 2983 | H | MET | A | 355 | 8.521 | 57.028 | 6.661 | 1.00 | 0.00 | H |
| ATOM | 2984 | HA | MET | A | 355 | 5.779 | 55.916 | 6.900 | 1.00 | 0.00 | H |
| ATOM | 2985 | 1HB | MET | A | 355 | 8.263 | 54.579 | 5.820 | 1.00 | 0.00 | H |
| ATOM | 2986 | 2HB | MET | A | 355 | 6.654 | 53.844 | 6.013 | 1.00 | 0.00 | H |
| ATOM | 2987 | 1HG | MET | A | 355 | 5.720 | 55.482 | 4.414 | 1.00 | 0.00 | H |
| ATOM | 2988 | 2HG | MET | A | 355 | 7.336 | 56.201 | 4.214 | 1.00 | 0.00 | H |
| ATOM | 2989 | 1HE | MET | A | 355 | 5.994 | 52.064 | 2.640 | 1.00 | 0.00 | H |
| ATOM | 2990 | 2HE | MET | A | 355 | 6.100 | 52.411 | 4.382 | 1.00 | 0.00 | H |
| ATOM | 2991 | 3HE | MET | A | 355 | 4.909 | 53.268 | 3.375 | 1.00 | 0.00 | H |
| ATOM | 2992 | N | GLY | A | 356 | 7.888 | 56.179 | 9.113 | 1.00 | 0.00 | N |
| ATOM | 2993 | CA | GLY | A | 356 | 8.304 | 55.964 | 10.491 | 1.00 | 0.00 | C |
| ATOM | 2994 | C | GLY | A | 356 | 8.948 | 54.600 | 10.765 | 1.00 | 0.00 | C |
| ATOM | 2995 | O | GLY | A | 356 | 8.502 | 53.867 | 11.649 | 1.00 | 0.00 | O |
| ATOM | 2996 | H | GLY | A | 356 | 8.070 | 57.114 | 8.778 | 1.00 | 0.00 | H |
| ATOM | 2997 | 1HA | GLY | A | 356 | 9.038 | 56.719 | 10.771 | 1.00 | 0.00 | H |
| ATOM | 2998 | 2HA | GLY | A | 356 | 7.437 | 56.038 | 11.148 | 1.00 | 0.00 | H |
| ATOM | 2999 | N | PHE | A | 357 | 10.059 | 54.285 | 10.095 | 1.00 | 0.00 | N |
| ATOM | 3000 | CA | PHE | A | 357 | 10.764 | 53.026 | 10.303 | 1.00 | 0.00 | C |
| ATOM | 3001 | C | PHE | A | 357 | 11.683 | 53.096 | 11.529 | 1.00 | 0.00 | C |
| ATOM | 3002 | O | PHE | A | 357 | 12.416 | 52.142 | 11.788 | 1.00 | 0.00 | O |
| ATOM | 3003 | CB | PHE | A | 357 | 11.586 | 52.633 | 9.063 | 1.00 | 0.00 | C |
| ATOM | 3004 | CG | PHE | A | 357 | 10.851 | 52.353 | 7.762 | 1.00 | 0.00 | C |
| ATOM | 3005 | CD1 | PHE | A | 357 | 9.461 | 52.125 | 7.717 | 1.00 | 0.00 | C |
| ATOM | 3006 | CD2 | PHE | A | 357 | 11.578 | 52.296 | 6.556 | 1.00 | 0.00 | C |
| ATOM | 3007 | CE1 | PHE | A | 357 | 8.814 | 51.861 | 6.499 | 1.00 | 0.00 | C |
| ATOM | 3008 | CE2 | PHE | A | 357 | 10.933 | 52.047 | 5.331 | 1.00 | 0.00 | C |
| ATOM | 3009 | CZ | PHE | A | 357 | 9.547 | 51.830 | 5.301 | 1.00 | 0.00 | C |
| ATOM | 3010 | H | PHE | A | 357 | 10.440 | 54.929 | 9.416 | 1.00 | 0.00 | H |
| ATOM | 3011 | HA | PHE | A | 357 | 10.042 | 52.217 | 10.408 | 1.00 | 0.00 | H |
| ATOM | 3012 | 1HB | PHE | A | 357 | 12.284 | 53.434 | 8.820 | 1.00 | 0.00 | H |
| ATOM | 3013 | 2HB | PHE | A | 357 | 12.141 | 51.718 | 9.270 | 1.00 | 0.00 | H |
| ATOM | 3014 | HD1 | PHE | A | 357 | 8.880 | 52.152 | 8.627 | 1.00 | 0.00 | H |
| ATOM | 3015 | HD2 | PHE | A | 357 | 12.648 | 52.446 | 6.563 | 1.00 | 0.00 | H |
| ATOM | 3016 | HE1 | PHE | A | 357 | 7.749 | 51.681 | 6.480 | 1.00 | 0.00 | H |
| ATOM | 3017 | HE2 | PHE | A | 357 | 11.504 | 52.022 | 4.415 | 1.00 | 0.00 | H |
| ATOM | 3018 | HZ | PHE | A | 357 | 9.043 | 51.641 | 4.365 | 1.00 | 0.00 | H |
| ATOM | 3019 | N | GLU | A | 358 | 11.679 | 54.215 | 12.259 | 1.00 | 0.00 | N |
| ATOM | 3020 | CA | GLU | A | 358 | 12.470 | 54.436 | 13.465 | 1.00 | 0.00 | C |
| ATOM | 3021 | C | GLU | A | 358 | 12.538 | 53.195 | 14.376 | 1.00 | 0.00 | C |
| ATOM | 3022 | O | GLU | A | 358 | 13.627 | 52.814 | 14.806 | 1.00 | 0.00 | O |
| ATOM | 3023 | CB | GLU | A | 358 | 11.978 | 55.693 | 14.204 | 1.00 | 0.00 | C |
| ATOM | 3024 | CG | GLU | A | 358 | 12.836 | 56.921 | 13.874 | 1.00 | 0.00 | C |
| ATOM | 3025 | CD | GLU | A | 358 | 14.110 | 56.912 | 14.701 | 1.00 | 0.00 | C |
| ATOM | 3026 | OE1 | GLU | A | 358 | 14.998 | 56.088 | 14.408 | 1.00 | 0.00 | O |
| ATOM | 3027 | OE2 | GLU | A | 358 | 14.170 | 57.667 | 15.693 | 1.00 | 0.00 | O1− |
| ATOM | 3028 | H | GLU | A | 358 | 11.095 | 54.993 | 11.987 | 1.00 | 0.00 | H |
| ATOM | 3029 | HA | GLU | A | 358 | 13.451 | 54.825 | 13.191 | 1.00 | 0.00 | H |
| ATOM | 3030 | 1HB | GLU | A | 358 | 10.949 | 55.907 | 13.914 | 1.00 | 0.00 | H |
| ATOM | 3031 | 2HB | GLU | A | 358 | 12.024 | 55.524 | 15.280 | 1.00 | 0.00 | H |
| ATOM | 3032 | 1HG | GLU | A | 358 | 13.098 | 56.908 | 12.816 | 1.00 | 0.00 | H |
| ATOM | 3033 | 2HG | GLU | A | 358 | 12.274 | 57.828 | 14.097 | 1.00 | 0.00 | H |
| ATOM | 3034 | N | PRO | A | 359 | 11.425 | 52.494 | 14.654 | 1.00 | 0.00 | N |
| ATOM | 3035 | CA | PRO | A | 359 | 11.476 | 51.384 | 15.592 | 1.00 | 0.00 | C |
| ATOM | 3036 | C | PRO | A | 359 | 12.271 | 50.196 | 15.033 | 1.00 | 0.00 | C |
| ATOM | 3037 | O | PRO | A | 359 | 12.624 | 49.279 | 15.771 | 1.00 | 0.00 | O |
| ATOM | 3038 | CB | PRO | A | 359 | 10.018 | 51.013 | 15.880 | 1.00 | 0.00 | C |
| ATOM | 3039 | CG | PRO | A | 359 | 9.272 | 52.324 | 15.625 | 1.00 | 0.00 | C |
| ATOM | 3040 | CD | PRO | A | 359 | 10.054 | 52.944 | 14.468 | 1.00 | 0.00 | C |
| ATOM | 3041 | HA | PRO | A | 359 | 11.918 | 51.720 | 16.530 | 1.00 | 0.00 | H |
| ATOM | 3042 | 1HB | PRO | A | 359 | 9.705 | 50.216 | 15.206 | 1.00 | 0.00 | H |
| ATOM | 3043 | 2HB | PRO | A | 359 | 9.926 | 50.673 | 16.912 | 1.00 | 0.00 | H |
| ATOM | 3044 | 1HG | PRO | A | 359 | 8.236 | 52.108 | 15.363 | 1.00 | 0.00 | H |
| ATOM | 3045 | 2HG | PRO | A | 359 | 9.298 | 52.939 | 16.525 | 1.00 | 0.00 | H |
| ATOM | 3046 | 1HD | PRO | A | 359 | 10.021 | 54.033 | 14.499 | 1.00 | 0.00 | H |
| ATOM | 3047 | 2HD | PRO | A | 359 | 9.685 | 52.597 | 13.503 | 1.00 | 0.00 | H |
| ATOM | 3048 | N | GLN | A | 360 | 12.545 | 50.203 | 13.727 | 1.00 | 0.00 | N |
| ATOM | 3049 | CA | GLN | A | 360 | 13.457 | 49.273 | 13.093 | 1.00 | 0.00 | C |
| ATOM | 3050 | C | GLN | A | 360 | 14.850 | 49.894 | 12.992 | 1.00 | 0.00 | C |
| ATOM | 3051 | O | GLN | A | 360 | 15.835 | 49.180 | 13.148 | 1.00 | 0.00 | O |
| ATOM | 3052 | CB | GLN | A | 360 | 12.962 | 48.852 | 11.705 | 1.00 | 0.00 | C |
| ATOM | 3053 | CG | GLN | A | 360 | 11.522 | 48.326 | 11.743 | 1.00 | 0.00 | C |
| ATOM | 3054 | CD | GLN | A | 360 | 10.537 | 49.380 | 11.259 | 1.00 | 0.00 | C |
| ATOM | 3055 | NE2 | GLN | A | 360 | 9.891 | 50.096 | 12.168 | 1.00 | 0.00 | N |
| ATOM | 3056 | OE1 | GLN | A | 360 | 10.371 | 49.553 | 10.058 | 1.00 | 0.00 | O |
| ATOM | 3057 | H | GLN | A | 360 | 12.106 | 50.884 | 13.123 | 1.00 | 0.00 | H |
| ATOM | 3058 | HA | GLN | A | 360 | 13.477 | 48.341 | 13.658 | 1.00 | 0.00 | H |
| ATOM | 3059 | 1HB | GLN | A | 360 | 12.992 | 49.710 | 11.033 | 1.00 | 0.00 | H |
| ATOM | 3060 | 2HB | GLN | A | 360 | 13.602 | 48.062 | 11.314 | 1.00 | 0.00 | H |
| ATOM | 3061 | 1HG | GLN | A | 360 | 11.438 | 47.450 | 11.100 | 1.00 | 0.00 | H |
| ATOM | 3062 | 2HG | GLN | A | 360 | 11.262 | 48.052 | 12.765 | 1.00 | 0.00 | H |
| ATOM | 3063 | 1HE2 | GLN | A | 360 | 9.231 | 50.803 | 11.878 | 1.00 | 0.00 | H |
| ATOM | 3064 | 2HE2 | GLN | A | 360 | 10.060 | 49.934 | 13.151 | 1.00 | 0.00 | H |
| ATOM | 3065 | N | ILE | A | 361 | 14.949 | 51.190 | 12.688 | 1.00 | 0.00 | N |
| ATOM | 3066 | CA | ILE | A | 361 | 16.239 | 51.850 | 12.536 | 1.00 | 0.00 | C |
| ATOM | 3067 | C | ILE | A | 361 | 17.000 | 51.782 | 13.860 | 1.00 | 0.00 | C |
| ATOM | 3068 | O | ILE | A | 361 | 18.152 | 51.360 | 13.884 | 1.00 | 0.00 | O |
| ATOM | 3069 | CB | ILE | A | 361 | 16.115 | 53.295 | 12.012 | 1.00 | 0.00 | C |
| ATOM | 3070 | CG1 | ILE | A | 361 | 15.418 | 53.341 | 10.639 | 1.00 | 0.00 | C |
| ATOM | 3071 | CG2 | ILE | A | 361 | 17.512 | 53.922 | 11.876 | 1.00 | 0.00 | C |
| ATOM | 3072 | CD1 | ILE | A | 361 | 15.024 | 54.766 | 10.232 | 1.00 | 0.00 | C |
| ATOM | 3073 | H | ILE | A | 361 | 14.115 | 51.744 | 12.557 | 1.00 | 0.00 | H |
| ATOM | 3074 | HA | ILE | A | 361 | 16.776 | 51.410 | 11.695 | 1.00 | 0.00 | H |
| ATOM | 3075 | HB | ILE | A | 361 | 15.538 | 53.890 | 12.720 | 1.00 | 0.00 | H |
| ATOM | 3076 | 1HG1 | ILE | A | 361 | 16.090 | 52.948 | 9.876 | 1.00 | 0.00 | H |
| ATOM | 3077 | 2HG1 | ILE | A | 361 | 14.511 | 52.738 | 10.670 | 1.00 | 0.00 | H |
| ATOM | 3078 | 1HG2 | ILE | A | 361 | 17.418 | 54.943 | 11.506 | 1.00 | 0.00 | H |
| ATOM | 3079 | 2HG2 | ILE | A | 361 | 18.002 | 53.933 | 12.850 | 1.00 | 0.00 | H |
| ATOM | 3080 | 3HG2 | ILE | A | 361 | 18.108 | 53.336 | 11.177 | 1.00 | 0.00 | H |
| ATOM | 3081 | 1HD1 | ILE | A | 361 | 14.537 | 54.745 | 9.257 | 1.00 | 0.00 | H |
| ATOM | 3082 | 2HD1 | ILE | A | 361 | 14.338 | 55.179 | 10.972 | 1.00 | 0.00 | H |
| ATOM | 3083 | 3HD1 | ILE | A | 361 | 15.917 | 55.389 | 10.178 | 1.00 | 0.00 | H |
| ATOM | 3084 | N | ARG | A | 362 | 16.385 | 52.179 | 14.972 | 1.00 | 0.00 | N |
| ATOM | 3085 | CA | ARG | A | 362 | 17.072 | 52.146 | 16.256 | 1.00 | 0.00 | C |
| ATOM | 3086 | C | ARG | A | 362 | 17.515 | 50.714 | 16.557 | 1.00 | 0.00 | C |
| ATOM | 3087 | O | ARG | A | 362 | 18.610 | 50.486 | 17.067 | 1.00 | 0.00 | O |
| ATOM | 3088 | CB | ARG | A | 362 | 16.167 | 52.673 | 17.375 | 1.00 | 0.00 | C |
| ATOM | 3089 | CG | ARG | A | 362 | 15.727 | 54.125 | 17.169 | 1.00 | 0.00 | C |
| ATOM | 3090 | CD | ARG | A | 362 | 16.871 | 55.136 | 17.304 | 1.00 | 0.00 | C |
| ATOM | 3091 | NE | ARG | A | 362 | 16.352 | 56.475 | 17.006 | 1.00 | 0.00 | N |
| ATOM | 3092 | CZ | ARG | A | 362 | 16.677 | 57.622 | 17.608 | 1.00 | 0.00 | C |
| ATOM | 3093 | NH1 | ARG | A | 362 | 17.749 | 57.714 | 18.389 | 1.00 | 0.00 | N1+ |
| ATOM | 3094 | NH2 | ARG | A | 362 | 15.898 | 58.678 | 17.436 | 1.00 | 0.00 | N |
| ATOM | 3095 | H | ARG | A | 362 | 15.431 | 52.508 | 14.932 | 1.00 | 0.00 | H |
| ATOM | 3096 | HA | ARG | A | 362 | 17.923 | 52.826 | 16.231 | 1.00 | 0.00 | H |
| ATOM | 3097 | 1HB | ARG | A | 362 | 15.266 | 52.062 | 17.432 | 1.00 | 0.00 | H |
| ATOM | 3098 | 2HB | ARG | A | 362 | 16.699 | 52.624 | 18.325 | 1.00 | 0.00 | H |
| ATOM | 3099 | 1HG | ARG | A | 362 | 15.307 | 54.239 | 16.169 | 1.00 | 0.00 | H |
| ATOM | 3100 | 2HG | ARG | A | 362 | 14.972 | 54.385 | 17.911 | 1.00 | 0.00 | H |
| ATOM | 3101 | 1HD | ARG | A | 362 | 17.262 | 55.109 | 18.321 | 1.00 | 0.00 | H |
| ATOM | 3102 | 2HD | ARG | A | 362 | 17.665 | 54.883 | 16.603 | 1.00 | 0.00 | H |
| ATOM | 3103 | HE | ARG | A | 362 | 15.678 | 56.472 | 16.254 | 1.00 | 0.00 | H |
| ATOM | 3104 | 1HH1 | ARG | A | 362 | 17.975 | 58.592 | 18.834 | 1.00 | 0.00 | H |
| ATOM | 3105 | 2HH1 | ARG | A | 362 | 18.337 | 56.907 | 18.537 | 1.00 | 0.00 | H |
| ATOM | 3106 | 1HH2 | ARG | A | 362 | 16.129 | 59.554 | 17.884 | 1.00 | 0.00 | H |
| ATOM | 3107 | 2HH2 | ARG | A | 362 | 15.074 | 58.609 | 16.857 | 1.00 | 0.00 | H |
| ATOM | 3108 | N | ARG | A | 363 | 16.676 | 49.750 | 16.177 | 1.00 | 0.00 | N |
| ATOM | 3109 | CA | ARG | A | 363 | 16.873 | 48.321 | 16.378 | 1.00 | 0.00 | C |
| ATOM | 3110 | C | ARG | A | 363 | 17.874 | 47.754 | 15.355 | 1.00 | 0.00 | C |
| ATOM | 3111 | O | ARG | A | 363 | 18.016 | 46.543 | 15.204 | 1.00 | 0.00 | O |
| ATOM | 3112 | CB | ARG | A | 363 | 15.477 | 47.664 | 16.344 | 1.00 | 0.00 | C |
| ATOM | 3113 | CG | ARG | A | 363 | 15.376 | 46.138 | 16.489 | 1.00 | 0.00 | C |
| ATOM | 3114 | CD | ARG | A | 363 | 15.956 | 45.585 | 17.802 | 1.00 | 0.00 | C |
| ATOM | 3115 | NE | ARG | A | 363 | 16.689 | 44.328 | 17.587 | 1.00 | 0.00 | N |
| ATOM | 3116 | CZ | ARG | A | 363 | 16.193 | 43.181 | 17.123 | 1.00 | 0.00 | C |
| ATOM | 3117 | NH1 | ARG | A | 363 | 14.880 | 43.061 | 16.922 | 1.00 | 0.00 | N1+ |
| ATOM | 3118 | NH2 | ARG | A | 363 | 17.033 | 42.187 | 16.865 | 1.00 | 0.00 | N |
| ATOM | 3119 | H | ARG | A | 363 | 15.814 | 49.987 | 15.706 | 1.00 | 0.00 | H |
| ATOM | 3120 | HA | ARG | A | 363 | 17.164 | 48.136 | 17.412 | 1.00 | 0.00 | H |
| ATOM | 3121 | 1HB | ARG | A | 363 | 14.869 | 48.059 | 17.158 | 1.00 | 0.00 | H |
| ATOM | 3122 | 2HB | ARG | A | 363 | 14.995 | 47.884 | 15.392 | 1.00 | 0.00 | H |
| ATOM | 3123 | 1HG | ARG | A | 363 | 14.329 | 45.838 | 16.452 | 1.00 | 0.00 | H |
| ATOM | 3124 | 2HG | ARG | A | 363 | 15.920 | 45.659 | 15.674 | 1.00 | 0.00 | H |
| ATOM | 3125 | 1HD | ARG | A | 363 | 16.643 | 46.313 | 18.232 | 1.00 | 0.00 | H |
| ATOM | 3126 | 2HD | ARG | A | 363 | 15.145 | 45.393 | 18.505 | 1.00 | 0.00 | H |
| ATOM | 3127 | HE | ARG | A | 363 | 17.667 | 44.393 | 17.832 | 1.00 | 0.00 | H |
| ATOM | 3128 | 1HH1 | ARG | A | 363 | 14.500 | 42.194 | 16.570 | 1.00 | 0.00 | H |
| ATOM | 3129 | 2HH1 | ARG | A | 363 | 14.266 | 43.838 | 17.121 | 1.00 | 0.00 | H |
| ATOM | 3130 | 1HH2 | ARG | A | 363 | 16.683 | 41.308 | 16.513 | 1.00 | 0.00 | H |
| ATOM | 3131 | 2HH2 | ARG | A | 363 | 18.024 | 42.310 | 17.021 | 1.00 | 0.00 | H |
| ATOM | 3132 | N | ILE | A | 364 | 18.590 | 48.629 | 14.653 | 1.00 | 0.00 | N |
| ATOM | 3133 | CA | ILE | A | 364 | 19.730 | 48.312 | 13.817 | 1.00 | 0.00 | C |
| ATOM | 3134 | C | ILE | A | 364 | 20.906 | 49.146 | 14.315 | 1.00 | 0.00 | C |
| ATOM | 3135 | O | ILE | A | 364 | 22.031 | 48.660 | 14.389 | 1.00 | 0.00 | O |
| ATOM | 3136 | CB | ILE | A | 364 | 19.398 | 48.646 | 12.349 | 1.00 | 0.00 | C |
| ATOM | 3137 | CG1 | ILE | A | 364 | 18.377 | 47.641 | 11.796 | 1.00 | 0.00 | C |
| ATOM | 3138 | CG2 | ILE | A | 364 | 20.656 | 48.692 | 11.471 | 1.00 | 0.00 | C |
| ATOM | 3139 | CD1 | ILE | A | 364 | 17.721 | 48.163 | 10.516 | 1.00 | 0.00 | C |
| ATOM | 3140 | H | ILE | A | 364 | 18.352 | 49.611 | 14.677 | 1.00 | 0.00 | H |
| ATOM | 3141 | HA | ILE | A | 364 | 19.948 | 47.247 | 13.888 | 1.00 | 0.00 | H |
| ATOM | 3142 | HB | ILE | A | 364 | 19.045 | 49.675 | 12.281 | 1.00 | 0.00 | H |
| ATOM | 3143 | 1HG1 | ILE | A | 364 | 18.879 | 46.701 | 11.569 | 1.00 | 0.00 | H |
| ATOM | 3144 | 2HG1 | ILE | A | 364 | 17.599 | 47.467 | 12.538 | 1.00 | 0.00 | H |
| ATOM | 3145 | 1HG2 | ILE | A | 364 | 20.376 | 48.931 | 10.445 | 1.00 | 0.00 | H |
| ATOM | 3146 | 2HG2 | ILE | A | 364 | 21.336 | 49.456 | 11.848 | 1.00 | 0.00 | H |
| ATOM | 3147 | 3HG2 | ILE | A | 364 | 21.152 | 47.721 | 11.495 | 1.00 | 0.00 | H |
| ATOM | 3148 | 1HD1 | ILE | A | 364 | 17.003 | 47.429 | 10.149 | 1.00 | 0.00 | H |
| ATOM | 3149 | 2HD1 | ILE | A | 364 | 17.205 | 49.100 | 10.728 | 1.00 | 0.00 | H |
| ATOM | 3150 | 3HD1 | ILE | A | 364 | 18.485 | 48.334 | 9.758 | 1.00 | 0.00 | H |
| ATOM | 3151 | N | VAL | A | 365 | 20.655 | 50.428 | 14.560 | 1.00 | 0.00 | N |
| ATOM | 3152 | CA | VAL | A | 365 | 21.681 | 51.448 | 14.570 | 1.00 | 0.00 | C |
| ATOM | 3153 | C | VAL | A | 365 | 22.255 | 51.656 | 15.973 | 1.00 | 0.00 | C |
| ATOM | 3154 | O | VAL | A | 365 | 23.406 | 52.085 | 16.087 | 1.00 | 0.00 | O |
| ATOM | 3155 | CB | VAL | A | 365 | 21.093 | 52.727 | 13.933 | 1.00 | 0.00 | C |
| ATOM | 3156 | CG1 | VAL | A | 365 | 22.007 | 53.949 | 14.066 | 1.00 | 0.00 | C |
| ATOM | 3157 | CG2 | VAL | A | 365 | 20.812 | 52.481 | 12.441 | 1.00 | 0.00 | C |
| ATOM | 3158 | H | VAL | A | 365 | 19.711 | 50.731 | 14.751 | 1.00 | 0.00 | H |
| ATOM | 3159 | HA | VAL | A | 365 | 22.439 | 51.209 | 13.824 | 1.00 | 0.00 | H |
| ATOM | 3160 | HB | VAL | A | 365 | 20.132 | 52.954 | 14.393 | 1.00 | 0.00 | H |
| ATOM | 3161 | 1HG1 | VAL | A | 365 | 21.532 | 54.811 | 13.597 | 1.00 | 0.00 | H |
| ATOM | 3162 | 2HG1 | VAL | A | 365 | 22.181 | 54.160 | 15.121 | 1.00 | 0.00 | H |
| ATOM | 3163 | 3HG1 | VAL | A | 365 | 22.958 | 53.747 | 13.575 | 1.00 | 0.00 | H |
| ATOM | 3164 | 1HG2 | VAL | A | 365 | 20.397 | 53.385 | 11.995 | 1.00 | 0.00 | H |
| ATOM | 3165 | 2HG2 | VAL | A | 365 | 21.741 | 52.219 | 11.934 | 1.00 | 0.00 | H |
| ATOM | 3166 | 3HG2 | VAL | A | 365 | 20.098 | 51.664 | 12.336 | 1.00 | 0.00 | H |
| ATOM | 3167 | N | GLU | A | 366 | 21.475 | 51.407 | 17.030 | 1.00 | 0.00 | N |
| ATOM | 3168 | CA | GLU | A | 366 | 21.892 | 51.754 | 18.384 | 1.00 | 0.00 | C |
| ATOM | 3169 | C | GLU | A | 366 | 21.170 | 50.949 | 19.472 | 1.00 | 0.00 | C |
| ATOM | 3170 | O | GLU | A | 366 | 21.348 | 51.222 | 20.658 | 1.00 | 0.00 | O |
| ATOM | 3171 | CB | GLU | A | 366 | 21.800 | 53.281 | 18.601 | 1.00 | 0.00 | C |
| ATOM | 3172 | CG | GLU | A | 366 | 20.691 | 54.017 | 17.817 | 1.00 | 0.00 | C |
| ATOM | 3173 | CD | GLU | A | 366 | 20.937 | 55.516 | 17.727 | 1.00 | 0.00 | C |
| ATOM | 3174 | OE1 | GLU | A | 366 | 19.979 | 56.257 | 18.027 | 1.00 | 0.00 | O |
| ATOM | 3175 | OE2 | GLU | A | 366 | 22.066 | 55.893 | 17.325 | 1.00 | 0.00 | O1− |
| ATOM | 3176 | H | GLU | A | 366 | 20.575 | 50.968 | 16.901 | 1.00 | 0.00 | H |
| ATOM | 3177 | HA | GLU | A | 366 | 22.979 | 51.705 | 18.454 | 1.00 | 0.00 | H |
| ATOM | 3178 | 1HB | GLU | A | 366 | 21.611 | 53.489 | 19.654 | 1.00 | 0.00 | H |
| ATOM | 3179 | 2HB | GLU | A | 366 | 22.738 | 53.749 | 18.302 | 1.00 | 0.00 | H |
| ATOM | 3180 | 1HG | GLU | A | 366 | 20.641 | 53.624 | 16.801 | 1.00 | 0.00 | H |
| ATOM | 3181 | 2HG | GLU | A | 366 | 19.732 | 53.864 | 18.313 | 1.00 | 0.00 | H |
| ATOM | 3182 | N | GLN | A | 367 | 20.351 | 49.966 | 19.100 | 1.00 | 0.00 | N |
| ATOM | 3183 | CA | GLN | A | 367 | 19.615 | 49.130 | 20.038 | 1.00 | 0.00 | C |
| ATOM | 3184 | C | GLN | A | 367 | 19.646 | 47.681 | 19.547 | 1.00 | 0.00 | C |
| ATOM | 3185 | O | GLN | A | 367 | 18.634 | 46.985 | 19.551 | 1.00 | 0.00 | O |
| ATOM | 3186 | CB | GLN | A | 367 | 18.181 | 49.651 | 20.222 | 1.00 | 0.00 | C |
| ATOM | 3187 | CG | GLN | A | 367 | 18.148 | 51.124 | 20.655 | 1.00 | 0.00 | C |
| ATOM | 3188 | CD | GLN | A | 367 | 16.731 | 51.618 | 20.909 | 1.00 | 0.00 | C |
| ATOM | 3189 | NE2 | GLN | A | 367 | 16.571 | 52.919 | 21.116 | 1.00 | 0.00 | N |
| ATOM | 3190 | OE1 | GLN | A | 367 | 15.777 | 50.850 | 20.905 | 1.00 | 0.00 | O |
| ATOM | 3191 | H | GLN | A | 367 | 20.215 | 49.766 | 18.120 | 1.00 | 0.00 | H |
| ATOM | 3192 | HA | GLN | A | 367 | 20.034 | 49.248 | 21.038 | 1.00 | 0.00 | H |
| ATOM | 3193 | 1HB | GLN | A | 367 | 17.639 | 49.564 | 19.281 | 1.00 | 0.00 | H |
| ATOM | 3194 | 2HB | GLN | A | 367 | 17.675 | 49.063 | 20.988 | 1.00 | 0.00 | H |
| ATOM | 3195 | 1HG | GLN | A | 367 | 18.718 | 51.245 | 21.576 | 1.00 | 0.00 | H |
| ATOM | 3196 | 2HG | GLN | A | 367 | 18.587 | 51.743 | 19.873 | 1.00 | 0.00 | H |
| ATOM | 3197 | 1HE2 | GLN | A | 367 | 15.649 | 53.292 | 21.289 | 1.00 | 0.00 | H |
| ATOM | 3198 | 2HE2 | GLN | A | 367 | 17.371 | 53.535 | 21.101 | 1.00 | 0.00 | H |
| ATOM | 3199 | N | ASP | A | 368 | 20.839 | 47.234 | 19.155 | 1.00 | 0.00 | N |
| ATOM | 3200 | CA | ASP | A | 368 | 21.197 | 45.852 | 18.852 | 1.00 | 0.00 | C |
| ATOM | 3201 | C | ASP | A | 368 | 22.730 | 45.840 | 18.788 | 1.00 | 0.00 | C |
| ATOM | 3202 | O | ASP | A | 368 | 23.364 | 46.769 | 19.292 | 1.00 | 0.00 | O |
| ATOM | 3203 | CB | ASP | A | 368 | 20.551 | 45.362 | 17.545 | 1.00 | 0.00 | C |
| ATOM | 3204 | CG | ASP | A | 368 | 20.405 | 43.845 | 17.508 | 1.00 | 0.00 | C |
| ATOM | 3205 | OD1 | ASP | A | 368 | 21.432 | 43.157 | 17.336 | 1.00 | 0.00 | O |
| ATOM | 3206 | OD2 | ASP | A | 368 | 19.252 | 43.372 | 17.620 | 1.00 | 0.00 | O1− |
| ATOM | 3207 | H | ASP | A | 368 | 21.608 | 47.879 | 19.042 | 1.00 | 0.00 | H |
| ATOM | 3208 | HA | ASP | A | 368 | 20.787 | 45.194 | 19.618 | 1.00 | 0.00 | H |
| ATOM | 3209 | 1HB | ASP | A | 368 | 19.558 | 45.799 | 17.443 | 1.00 | 0.00 | H |
| ATOM | 3210 | 2HB | ASP | A | 368 | 21.169 | 45.662 | 16.699 | 1.00 | 0.00 | H |
| ATOM | 3211 | N | THR | A | 369 | 23.357 | 44.846 | 18.166 | 1.00 | 0.00 | N |
| ATOM | 3212 | CA | THR | A | 369 | 24.806 | 44.766 | 18.132 | 1.00 | 0.00 | C |
| ATOM | 3213 | C | THR | A | 369 | 25.354 | 45.662 | 17.007 | 1.00 | 0.00 | C |
| ATOM | 3214 | O | THR | A | 369 | 25.684 | 45.192 | 15.916 | 1.00 | 0.00 | O |
| ATOM | 3215 | CB | THR | A | 369 | 25.260 | 43.288 | 18.114 | 1.00 | 0.00 | C |
| ATOM | 3216 | CG2 | THR | A | 369 | 24.861 | 42.486 | 16.868 | 1.00 | 0.00 | C |
| ATOM | 3217 | OG1 | THR | A | 369 | 26.659 | 43.180 | 18.325 | 1.00 | 0.00 | O |
| ATOM | 3218 | H | THR | A | 369 | 22.825 | 44.124 | 17.702 | 1.00 | 0.00 | H |
| ATOM | 3219 | HA | THR | A | 369 | 25.203 | 44.937 | 19.132 | 1.00 | 0.00 | H |
| ATOM | 3220 | HB | THR | A | 369 | 24.915 | 42.790 | 19.020 | 1.00 | 0.00 | H |
| ATOM | 3221 | 1HG2 | THR | A | 369 | 25.230 | 41.464 | 16.960 | 1.00 | 0.00 | H |
| ATOM | 3222 | 2HG2 | THR | A | 369 | 23.775 | 42.473 | 16.776 | 1.00 | 0.00 | H |
| ATOM | 3223 | 3HG2 | THR | A | 369 | 25.295 | 42.950 | 15.982 | 1.00 | 0.00 | H |
| ATOM | 3224 | HG1 | THR | A | 369 | 26.916 | 42.255 | 18.309 | 1.00 | 0.00 | H |
| ATOM | 3225 | N | MET | A | 370 | 25.509 | 46.955 | 17.307 | 1.00 | 0.00 | N |
| ATOM | 3226 | CA | MET | A | 370 | 26.251 | 47.921 | 16.512 | 1.00 | 0.00 | C |
| ATOM | 3227 | C | MET | A | 370 | 26.904 | 48.917 | 17.478 | 1.00 | 0.00 | C |
| ATOM | 3228 | O | MET | A | 370 | 26.235 | 49.410 | 18.383 | 1.00 | 0.00 | O |
| ATOM | 3229 | CB | MET | A | 370 | 25.340 | 48.677 | 15.531 | 1.00 | 0.00 | C |
| ATOM | 3230 | CG | MET | A | 370 | 26.209 | 49.497 | 14.560 | 1.00 | 0.00 | C |
| ATOM | 3231 | SD | MET | A | 370 | 25.369 | 50.740 | 13.550 | 1.00 | 0.00 | S |
| ATOM | 3232 | CE | MET | A | 370 | 24.560 | 49.685 | 12.333 | 1.00 | 0.00 | C |
| ATOM | 3233 | H | MET | A | 370 | 25.092 | 47.332 | 18.146 | 1.00 | 0.00 | H |
| ATOM | 3234 | HA | MET | A | 370 | 27.009 | 47.402 | 15.924 | 1.00 | 0.00 | H |
| ATOM | 3235 | 1HB | MET | A | 370 | 24.738 | 47.964 | 14.969 | 1.00 | 0.00 | H |
| ATOM | 3236 | 2HB | MET | A | 370 | 24.683 | 49.347 | 16.087 | 1.00 | 0.00 | H |
| ATOM | 3237 | 1HG | MET | A | 370 | 26.967 | 50.042 | 15.123 | 1.00 | 0.00 | H |
| ATOM | 3238 | 2HG | MET | A | 370 | 26.696 | 48.826 | 13.853 | 1.00 | 0.00 | H |
| ATOM | 3239 | 1HE | MET | A | 370 | 23.997 | 50.303 | 11.633 | 1.00 | 0.00 | H |
| ATOM | 3240 | 2HE | MET | A | 370 | 25.312 | 49.114 | 11.790 | 1.00 | 0.00 | H |
| ATOM | 3241 | 3HE | MET | A | 370 | 23.881 | 49.000 | 12.840 | 1.00 | 0.00 | H |
| ATOM | 3242 | N | PRO | A | 371 | 28.191 | 49.248 | 17.309 | 1.00 | 0.00 | N |
| ATOM | 3243 | CA | PRO | A | 371 | 28.809 | 50.328 | 18.063 | 1.00 | 0.00 | C |
| ATOM | 3244 | C | PRO | A | 371 | 28.146 | 51.693 | 17.783 | 1.00 | 0.00 | C |
| ATOM | 3245 | O | PRO | A | 371 | 27.597 | 51.896 | 16.702 | 1.00 | 0.00 | O |
| ATOM | 3246 | CB | PRO | A | 371 | 30.267 | 50.336 | 17.595 | 1.00 | 0.00 | C |
| ATOM | 3247 | CG | PRO | A | 371 | 30.520 | 48.905 | 17.120 | 1.00 | 0.00 | C |
| ATOM | 3248 | CD | PRO | A | 371 | 29.169 | 48.511 | 16.530 | 1.00 | 0.00 | C |
| ATOM | 3249 | HA | PRO | A | 371 | 28.717 | 50.126 | 19.130 | 1.00 | 0.00 | H |
| ATOM | 3250 | 1HB | PRO | A | 371 | 30.387 | 51.060 | 16.790 | 1.00 | 0.00 | H |
| ATOM | 3251 | 2HB | PRO | A | 371 | 30.915 | 50.609 | 18.429 | 1.00 | 0.00 | H |
| ATOM | 3252 | 1HG | PRO | A | 371 | 31.321 | 48.904 | 16.381 | 1.00 | 0.00 | H |
| ATOM | 3253 | 2HG | PRO | A | 371 | 30.808 | 48.286 | 17.969 | 1.00 | 0.00 | H |
| ATOM | 3254 | 1HD | PRO | A | 371 | 28.983 | 47.442 | 16.631 | 1.00 | 0.00 | H |
| ATOM | 3255 | 2HD | PRO | A | 371 | 29.089 | 48.796 | 15.481 | 1.00 | 0.00 | H |
| ATOM | 3256 | N | PRO | A | 372 | 28.239 | 52.662 | 18.704 | 1.00 | 0.00 | N |
| ATOM | 3257 | CA | PRO | A | 372 | 27.552 | 53.941 | 18.564 | 1.00 | 0.00 | C |
| ATOM | 3258 | C | PRO | A | 372 | 28.309 | 54.890 | 17.620 | 1.00 | 0.00 | C |
| ATOM | 3259 | O | PRO | A | 372 | 29.373 | 54.544 | 17.105 | 1.00 | 0.00 | O |
| ATOM | 3260 | CB | PRO | A | 372 | 27.479 | 54.503 | 19.989 | 1.00 | 0.00 | C |
| ATOM | 3261 | CG | PRO | A | 372 | 28.706 | 53.897 | 20.669 | 1.00 | 0.00 | C |
| ATOM | 3262 | CD | PRO | A | 372 | 28.770 | 52.505 | 20.045 | 1.00 | 0.00 | C |
| ATOM | 3263 | HA | PRO | A | 372 | 26.549 | 53.774 | 18.172 | 1.00 | 0.00 | H |
| ATOM | 3264 | 1HB | PRO | A | 372 | 27.517 | 55.591 | 19.953 | 1.00 | 0.00 | H |
| ATOM | 3265 | 2HB | PRO | A | 372 | 26.546 | 54.187 | 20.457 | 1.00 | 0.00 | H |
| ATOM | 3266 | 1HG | PRO | A | 372 | 29.583 | 54.504 | 20.444 | 1.00 | 0.00 | H |
| ATOM | 3267 | 2HG | PRO | A | 372 | 28.550 | 53.872 | 21.747 | 1.00 | 0.00 | H |
| ATOM | 3268 | 1HD | PRO | A | 372 | 28.160 | 51.791 | 20.597 | 1.00 | 0.00 | H |
| ATOM | 3269 | 2HD | PRO | A | 372 | 29.796 | 52.139 | 19.989 | 1.00 | 0.00 | H |
| ATOM | 3270 | N | LYS | A | 373 | 27.735 | 56.077 | 17.399 | 1.00 | 0.00 | N |
| ATOM | 3271 | CA | LYS | A | 373 | 28.261 | 57.142 | 16.544 | 1.00 | 0.00 | C |
| ATOM | 3272 | C | LYS | A | 373 | 29.768 | 57.381 | 16.720 | 1.00 | 0.00 | C |
| ATOM | 3273 | O | LYS | A | 373 | 30.329 | 57.154 | 17.795 | 1.00 | 0.00 | O |
| ATOM | 3274 | CB | LYS | A | 373 | 27.426 | 58.424 | 16.694 | 1.00 | 0.00 | C |
| ATOM | 3275 | CG | LYS | A | 373 | 26.900 | 58.762 | 18.102 | 1.00 | 0.00 | C |
| ATOM | 3276 | CD | LYS | A | 373 | 26.113 | 60.074 | 18.000 | 1.00 | 0.00 | C |
| ATOM | 3277 | CE | LYS | A | 373 | 25.362 | 60.466 | 19.281 | 1.00 | 0.00 | C |
| ATOM | 3278 | NZ | LYS | A | 373 | 24.826 | 61.842 | 19.190 | 1.00 | 0.00 | N1+ |
| ATOM | 3279 | H | LYS | A | 373 | 26.856 | 56.303 | 17.842 | 1.00 | 0.00 | H |
| ATOM | 3280 | HA | LYS | A | 373 | 27.961 | 56.962 | 15.512 | 1.00 | 0.00 | H |
| ATOM | 3281 | 1HB | LYS | A | 373 | 28.023 | 59.285 | 16.392 | 1.00 | 0.00 | H |
| ATOM | 3282 | 2HB | LYS | A | 373 | 26.541 | 58.358 | 16.061 | 1.00 | 0.00 | H |
| ATOM | 3283 | 1HG | LYS | A | 373 | 26.253 | 57.958 | 18.451 | 1.00 | 0.00 | H |
| ATOM | 3284 | 2HG | LYS | A | 373 | 27.740 | 58.876 | 18.787 | 1.00 | 0.00 | H |
| ATOM | 3285 | 1HD | LYS | A | 373 | 26.796 | 60.892 | 17.771 | 1.00 | 0.00 | H |
| ATOM | 3286 | 2HD | LYS | A | 373 | 25.368 | 59.991 | 17.209 | 1.00 | 0.00 | H |
| ATOM | 3287 | 1HE | LYS | A | 373 | 24.530 | 59.780 | 19.439 | 1.00 | 0.00 | H |
| ATOM | 3288 | 2HE | LYS | A | 373 | 26.041 | 60.414 | 20.132 | 1.00 | 0.00 | H |
| ATOM | 3289 | 1HZ | LYS | A | 373 | 24.338 | 62.071 | 20.044 | 1.00 | 0.00 | H |
| ATOM | 3290 | 2HZ | LYS | A | 373 | 25.587 | 62.492 | 19.052 | 1.00 | 0.00 | H |
| ATOM | 3291 | 3HZ | LYS | A | 373 | 24.186 | 61.903 | 18.411 | 1.00 | 0.00 | H |
| ATOM | 3292 | N | GLY | A | 374 | 30.440 | 57.770 | 15.637 | 1.00 | 0.00 | N |
| ATOM | 3293 | CA | GLY | A | 374 | 31.887 | 57.783 | 15.532 | 1.00 | 0.00 | C |
| ATOM | 3294 | C | GLY | A | 374 | 32.440 | 56.360 | 15.356 | 1.00 | 0.00 | C |
| ATOM | 3295 | O | GLY | A | 374 | 33.177 | 56.059 | 14.406 | 1.00 | 0.00 | O |
| ATOM | 3296 | H | GLY | A | 374 | 29.940 | 58.082 | 14.817 | 1.00 | 0.00 | H |
| ATOM | 3297 | 1HA | GLY | A | 374 | 32.184 | 58.382 | 14.670 | 1.00 | 0.00 | H |
| ATOM | 3298 | 2HA | GLY | A | 374 | 32.314 | 58.215 | 16.437 | 1.00 | 0.00 | H |
| ATOM | 3299 | N | VAL | A | 375 | 32.125 | 55.466 | 16.298 | 1.00 | 0.00 | N |
| ATOM | 3300 | CA | VAL | A | 375 | 32.606 | 54.098 | 16.277 | 1.00 | 0.00 | C |
| ATOM | 3301 | C | VAL | A | 375 | 32.000 | 53.391 | 15.061 | 1.00 | 0.00 | C |
| ATOM | 3302 | O | VAL | A | 375 | 32.741 | 52.905 | 14.203 | 1.00 | 0.00 | O |
| ATOM | 3303 | CB | VAL | A | 375 | 32.357 | 53.371 | 17.612 | 1.00 | 0.00 | C |
| ATOM | 3304 | CG1 | VAL | A | 375 | 33.223 | 52.104 | 17.669 | 1.00 | 0.00 | C |
| ATOM | 3305 | CG2 | VAL | A | 375 | 32.705 | 54.254 | 18.819 | 1.00 | 0.00 | C |
| ATOM | 3306 | H | VAL | A | 375 | 31.527 | 55.732 | 17.067 | 1.00 | 0.00 | H |
| ATOM | 3307 | HA | VAL | A | 375 | 33.695 | 54.095 | 16.327 | 1.00 | 0.00 | H |
| ATOM | 3308 | HB | VAL | A | 375 | 31.304 | 53.099 | 17.688 | 1.00 | 0.00 | H |
| ATOM | 3309 | 1HG1 | VAL | A | 375 | 33.050 | 51.587 | 18.612 | 1.00 | 0.00 | H |
| ATOM | 3310 | 2HG1 | VAL | A | 375 | 32.961 | 51.447 | 16.840 | 1.00 | 0.00 | H |
| ATOM | 3311 | 3HG1 | VAL | A | 375 | 34.275 | 52.380 | 17.594 | 1.00 | 0.00 | H |
| ATOM | 3312 | 1HG2 | VAL | A | 375 | 32.515 | 53.702 | 19.740 | 1.00 | 0.00 | H |
| ATOM | 3313 | 2HG2 | VAL | A | 375 | 33.758 | 54.533 | 18.774 | 1.00 | 0.00 | H |
| ATOM | 3314 | 3HG2 | VAL | A | 375 | 32.090 | 55.153 | 18.801 | 1.00 | 0.00 | H |
| ATOM | 3315 | N | ARG | A | 376 | 30.675 | 53.404 | 14.931 | 1.00 | 0.00 | N |
| ATOM | 3316 | CA | ARG | A | 376 | 30.075 | 53.408 | 13.608 | 1.00 | 0.00 | C |
| ATOM | 3317 | C | ARG | A | 376 | 30.325 | 54.833 | 13.103 | 1.00 | 0.00 | C |
| ATOM | 3318 | O | ARG | A | 376 | 29.983 | 55.779 | 13.798 | 1.00 | 0.00 | O |
| ATOM | 3319 | CB | ARG | A | 376 | 28.569 | 53.050 | 13.662 | 1.00 | 0.00 | C |
| ATOM | 3320 | CG | ARG | A | 376 | 27.702 | 54.251 | 14.065 | 1.00 | 0.00 | C |
| ATOM | 3321 | CD | ARG | A | 376 | 26.233 | 54.021 | 14.412 | 1.00 | 0.00 | C |
| ATOM | 3322 | NE | ARG | A | 376 | 25.603 | 55.330 | 14.679 | 1.00 | 0.00 | N |
| ATOM | 3323 | CZ | ARG | A | 376 | 24.654 | 55.587 | 15.590 | 1.00 | 0.00 | C |
| ATOM | 3324 | NH1 | ARG | A | 376 | 24.169 | 54.624 | 16.359 | 1.00 | 0.00 | N1+ |
| ATOM | 3325 | NH2 | ARG | A | 376 | 24.172 | 56.812 | 15.756 | 1.00 | 0.00 | N |
| ATOM | 3326 | H | ARG | A | 376 | 30.087 | 53.410 | 15.752 | 1.00 | 0.00 | H |
| ATOM | 3327 | HA | ARG | A | 376 | 30.544 | 52.639 | 12.993 | 1.00 | 0.00 | H |
| ATOM | 3328 | 1HB | ARG | A | 376 | 28.240 | 52.710 | 12.680 | 1.00 | 0.00 | H |
| ATOM | 3329 | 2HB | ARG | A | 376 | 28.410 | 52.257 | 14.392 | 1.00 | 0.00 | H |
| ATOM | 3330 | 1HG | ARG | A | 376 | 28.122 | 54.721 | 14.955 | 1.00 | 0.00 | H |
| ATOM | 3331 | 2HG | ARG | A | 376 | 27.679 | 54.974 | 13.250 | 1.00 | 0.00 | H |
| ATOM | 3332 | 1HD | ARG | A | 376 | 25.734 | 53.532 | 13.576 | 1.00 | 0.00 | H |
| ATOM | 3333 | 2HD | ARG | A | 376 | 26.163 | 53.388 | 15.297 | 1.00 | 0.00 | H |
| ATOM | 3334 | HE | ARG | A | 376 | 25.954 | 56.074 | 14.092 | 1.00 | 0.00 | H |
| ATOM | 3335 | 1HH1 | ARG | A | 376 | 23.454 | 54.834 | 17.041 | 1.00 | 0.00 | H |
| ATOM | 3336 | 2HH1 | ARG | A | 376 | 24.514 | 53.679 | 16.264 | 1.00 | 0.00 | H |
| ATOM | 3337 | 1HH2 | ARG | A | 376 | 23.457 | 56.985 | 16.448 | 1.00 | 0.00 | H |
| ATOM | 3338 | 2HH2 | ARG | A | 376 | 24.521 | 57.573 | 15.190 | 1.00 | 0.00 | H |
| ATOM | 3339 | N | HIS | A | 377 | 30.919 | 55.028 | 11.931 | 1.00 | 0.00 | N |
| ATOM | 3340 | CA | HIS | A | 377 | 30.875 | 56.360 | 11.348 | 1.00 | 0.00 | C |
| ATOM | 3341 | C | HIS | A | 377 | 29.508 | 56.462 | 10.672 | 1.00 | 0.00 | C |
| ATOM | 3342 | O | HIS | A | 377 | 29.178 | 55.612 | 9.839 | 1.00 | 0.00 | O |
| ATOM | 3343 | CB | HIS | A | 377 | 32.040 | 56.568 | 10.379 | 1.00 | 0.00 | C |
| ATOM | 3344 | CG | HIS | A | 377 | 31.862 | 57.731 | 9.436 | 1.00 | 0.00 | C |
| ATOM | 3345 | CD2 | HIS | A | 377 | 32.231 | 57.902 | 8.127 | 1.00 | 0.00 | C |
| ATOM | 3346 | ND1 | HIS | A | 377 | 31.220 | 58.913 | 9.787 | 1.00 | 0.00 | N |
| ATOM | 3347 | CE1 | HIS | A | 377 | 31.127 | 59.676 | 8.684 | 1.00 | 0.00 | C |
| ATOM | 3348 | NE2 | HIS | A | 377 | 31.733 | 59.097 | 7.652 | 1.00 | 0.00 | N |
| ATOM | 3349 | H | HIS | A | 377 | 31.386 | 54.262 | 11.468 | 1.00 | 0.00 | H |
| ATOM | 3350 | HA | HIS | A | 377 | 31.033 | 57.104 | 12.129 | 1.00 | 0.00 | H |
| ATOM | 3351 | 1HB | HIS | A | 377 | 32.954 | 56.751 | 10.944 | 1.00 | 0.00 | H |
| ATOM | 3352 | 2HB | HIS | A | 377 | 32.165 | 55.676 | 9.765 | 1.00 | 0.00 | H |
| ATOM | 3353 | HD2 | HIS | A | 377 | 32.827 | 57.150 | 7.631 | 1.00 | 0.00 | H |
| ATOM | 3354 | HD1 | HIS | A | 377 | 30.920 | 59.061 | 10.740 | 1.00 | 0.00 | H |
| ATOM | 3355 | HE1 | HIS | A | 377 | 30.608 | 60.622 | 8.724 | 1.00 | 0.00 | H |
| ATOM | 3356 | N | THR | A | 378 | 28.722 | 57.458 | 11.057 | 1.00 | 0.00 | N |
| ATOM | 3357 | CA | THR | A | 378 | 27.334 | 57.609 | 10.672 | 1.00 | 0.00 | C |
| ATOM | 3358 | C | THR | A | 378 | 27.212 | 58.680 | 9.586 | 1.00 | 0.00 | C |
| ATOM | 3359 | O | THR | A | 378 | 27.745 | 59.784 | 9.720 | 1.00 | 0.00 | O |
| ATOM | 3360 | CB | THR | A | 378 | 26.491 | 57.982 | 11.904 | 1.00 | 0.00 | C |
| ATOM | 3361 | CG2 | THR | A | 378 | 25.017 | 57.653 | 11.655 | 1.00 | 0.00 | C |
| ATOM | 3362 | OG1 | THR | A | 378 | 26.930 | 57.312 | 13.072 | 1.00 | 0.00 | O |
| ATOM | 3363 | H | THR | A | 378 | 29.083 | 58.182 | 11.661 | 1.00 | 0.00 | H |
| ATOM | 3364 | HA | THR | A | 378 | 26.948 | 56.652 | 10.320 | 1.00 | 0.00 | H |
| ATOM | 3365 | HB | THR | A | 378 | 26.618 | 59.042 | 12.124 | 1.00 | 0.00 | H |
| ATOM | 3366 | 1HG2 | THR | A | 378 | 24.430 | 57.921 | 12.534 | 1.00 | 0.00 | H |
| ATOM | 3367 | 2HG2 | THR | A | 378 | 24.658 | 58.217 | 10.794 | 1.00 | 0.00 | H |
| ATOM | 3368 | 3HG2 | THR | A | 378 | 24.910 | 56.586 | 11.459 | 1.00 | 0.00 | H |
| ATOM | 3369 | HG1 | THR | A | 378 | 26.380 | 57.571 | 13.815 | 1.00 | 0.00 | H |
| ATOM | 3370 | N | MET | A | 379 | 26.496 | 58.357 | 8.509 | 1.00 | 0.00 | N |
| ATOM | 3371 | CA | MET | A | 379 | 26.322 | 59.222 | 7.359 | 1.00 | 0.00 | C |
| ATOM | 3372 | C | MET | A | 379 | 24.839 | 59.235 | 6.998 | 1.00 | 0.00 | C |
| ATOM | 3373 | O | MET | A | 379 | 24.299 | 58.228 | 6.542 | 1.00 | 0.00 | O |
| ATOM | 3374 | CB | MET | A | 379 | 27.147 | 58.655 | 6.201 | 1.00 | 0.00 | C |
| ATOM | 3375 | CG | MET | A | 379 | 28.654 | 58.689 | 6.464 | 1.00 | 0.00 | C |
| ATOM | 3376 | SD | MET | A | 379 | 29.600 | 57.565 | 5.406 | 1.00 | 0.00 | S |
| ATOM | 3377 | CE | MET | A | 379 | 29.529 | 56.067 | 6.417 | 1.00 | 0.00 | C |
| ATOM | 3378 | H | MET | A | 379 | 26.034 | 57.461 | 8.461 | 1.00 | 0.00 | H |
| ATOM | 3379 | HA | MET | A | 379 | 26.650 | 60.231 | 7.610 | 1.00 | 0.00 | H |
| ATOM | 3380 | 1HB | MET | A | 379 | 26.865 | 57.617 | 6.028 | 1.00 | 0.00 | H |
| ATOM | 3381 | 2HB | MET | A | 379 | 26.956 | 59.238 | 5.299 | 1.00 | 0.00 | H |
| ATOM | 3382 | 1HG | MET | A | 379 | 29.032 | 59.697 | 6.287 | 1.00 | 0.00 | H |
| ATOM | 3383 | 2HG | MET | A | 379 | 28.850 | 58.404 | 7.498 | 1.00 | 0.00 | H |
| ATOM | 3384 | 1HE | MET | A | 379 | 30.066 | 55.263 | 5.914 | 1.00 | 0.00 | H |
| ATOM | 3385 | 2HE | MET | A | 379 | 29.989 | 56.260 | 7.386 | 1.00 | 0.00 | H |
| ATOM | 3386 | 3HE | MET | A | 379 | 28.489 | 55.774 | 6.561 | 1.00 | 0.00 | H |
| ATOM | 3387 | N | MET | A | 380 | 24.180 | 60.373 | 7.182 | 1.00 | 0.00 | N |
| ATOM | 3388 | CA | MET | A | 380 | 22.786 | 60.549 | 6.826 | 1.00 | 0.00 | C |
| ATOM | 3389 | C | MET | A | 380 | 22.740 | 61.327 | 5.517 | 1.00 | 0.00 | C |
| ATOM | 3390 | O | MET | A | 380 | 23.243 | 62.447 | 5.452 | 1.00 | 0.00 | O |
| ATOM | 3391 | CB | MET | A | 380 | 22.072 | 61.341 | 7.926 | 1.00 | 0.00 | C |
| ATOM | 3392 | CG | MET | A | 380 | 21.909 | 60.537 | 9.217 | 1.00 | 0.00 | C |
| ATOM | 3393 | SD | MET | A | 380 | 21.330 | 61.515 | 10.631 | 1.00 | 0.00 | S |
| ATOM | 3394 | CE | MET | A | 380 | 19.605 | 61.799 | 10.162 | 1.00 | 0.00 | C |
| ATOM | 3395 | H | MET | A | 380 | 24.651 | 61.168 | 7.589 | 1.00 | 0.00 | H |
| ATOM | 3396 | HA | MET | A | 380 | 22.318 | 59.573 | 6.692 | 1.00 | 0.00 | H |
| ATOM | 3397 | 1HB | MET | A | 380 | 22.646 | 62.238 | 8.160 | 1.00 | 0.00 | H |
| ATOM | 3398 | 2HB | MET | A | 380 | 21.078 | 61.627 | 7.582 | 1.00 | 0.00 | H |
| ATOM | 3399 | 1HG | MET | A | 380 | 21.183 | 59.740 | 9.060 | 1.00 | 0.00 | H |
| ATOM | 3400 | 2HG | MET | A | 380 | 22.869 | 60.104 | 9.499 | 1.00 | 0.00 | H |
| ATOM | 3401 | 1HE | MET | A | 380 | 19.110 | 62.389 | 10.934 | 1.00 | 0.00 | H |
| ATOM | 3402 | 2HE | MET | A | 380 | 19.569 | 62.338 | 9.215 | 1.00 | 0.00 | H |
| ATOM | 3403 | 3HE | MET | A | 380 | 19.094 | 60.842 | 10.055 | 1.00 | 0.00 | H |
| ATOM | 3404 | N | PHE | A | 381 | 22.097 | 60.758 | 4.502 | 1.00 | 0.00 | N |
| ATOM | 3405 | CA | PHE | A | 381 | 21.719 | 61.462 | 3.296 | 1.00 | 0.00 | C |
| ATOM | 3406 | C | PHE | A | 381 | 20.194 | 61.421 | 3.226 | 1.00 | 0.00 | C |
| ATOM | 3407 | O | PHE | A | 381 | 19.572 | 60.373 | 3.417 | 1.00 | 0.00 | O |
| ATOM | 3408 | CB | PHE | A | 381 | 22.337 | 60.808 | 2.052 | 1.00 | 0.00 | C |
| ATOM | 3409 | CG | PHE | A | 381 | 23.849 | 60.866 | 1.944 | 1.00 | 0.00 | C |
| ATOM | 3410 | CD1 | PHE | A | 381 | 24.477 | 61.822 | 1.123 | 1.00 | 0.00 | C |
| ATOM | 3411 | CD2 | PHE | A | 381 | 24.649 | 59.949 | 2.650 | 1.00 | 0.00 | C |
| ATOM | 3412 | CE1 | PHE | A | 381 | 25.877 | 61.853 | 0.994 | 1.00 | 0.00 | C |
| ATOM | 3413 | CE2 | PHE | A | 381 | 26.050 | 59.985 | 2.536 | 1.00 | 0.00 | C |
| ATOM | 3414 | CZ | PHE | A | 381 | 26.667 | 60.936 | 1.706 | 1.00 | 0.00 | C |
| ATOM | 3415 | H | PHE | A | 381 | 21.846 | 59.780 | 4.547 | 1.00 | 0.00 | H |
| ATOM | 3416 | HA | PHE | A | 381 | 22.090 | 62.485 | 3.341 | 1.00 | 0.00 | H |
| ATOM | 3417 | 1HB | PHE | A | 381 | 22.075 | 59.751 | 2.028 | 1.00 | 0.00 | H |
| ATOM | 3418 | 2HB | PHE | A | 381 | 21.954 | 61.297 | 1.156 | 1.00 | 0.00 | H |
| ATOM | 3419 | HD1 | PHE | A | 381 | 23.883 | 62.543 | 0.582 | 1.00 | 0.00 | H |
| ATOM | 3420 | HD2 | PHE | A | 381 | 24.188 | 59.209 | 3.287 | 1.00 | 0.00 | H |
| ATOM | 3421 | HE1 | PHE | A | 381 | 26.344 | 62.582 | 0.348 | 1.00 | 0.00 | H |
| ATOM | 3422 | HE2 | PHE | A | 381 | 26.654 | 59.280 | 3.088 | 1.00 | 0.00 | H |
| ATOM | 3423 | HZ | PHE | A | 381 | 27.743 | 60.964 | 1.614 | 1.00 | 0.00 | H |
| ATOM | 3424 | N | SER | A | 382 | 19.591 | 62.561 | 2.929 | 1.00 | 0.00 | N |
| ATOM | 3425 | CA | SER | A | 382 | 18.168 | 62.687 | 2.705 | 1.00 | 0.00 | C |
| ATOM | 3426 | C | SER | A | 382 | 18.019 | 63.660 | 1.544 | 1.00 | 0.00 | C |
| ATOM | 3427 | O | SER | A | 382 | 18.862 | 64.537 | 1.340 | 1.00 | 0.00 | O |
| ATOM | 3428 | CB | SER | A | 382 | 17.462 | 63.176 | 3.977 | 1.00 | 0.00 | C |
| ATOM | 3429 | OG | SER | A | 382 | 18.131 | 64.276 | 4.570 | 1.00 | 0.00 | O |
| ATOM | 3430 | H | SER | A | 382 | 20.131 | 63.411 | 2.847 | 1.00 | 0.00 | H |
| ATOM | 3431 | HA | SER | A | 382 | 17.745 | 61.705 | 2.497 | 1.00 | 0.00 | H |
| ATOM | 3432 | 1HB | SER | A | 382 | 16.447 | 63.489 | 3.731 | 1.00 | 0.00 | H |
| ATOM | 3433 | 2HB | SER | A | 382 | 17.425 | 62.367 | 4.707 | 1.00 | 0.00 | H |
| ATOM | 3434 | HG | SER | A | 382 | 17.659 | 64.550 | 5.359 | 1.00 | 0.00 | H |
| ATOM | 3435 | N | ALA | A | 383 | 16.977 | 63.497 | 0.746 | 1.00 | 0.00 | N |
| ATOM | 3436 | CA | ALA | A | 383 | 16.718 | 64.414 | −0.341 | 1.00 | 0.00 | C |
| ATOM | 3437 | C | ALA | A | 383 | 16.056 | 65.694 | 0.189 | 1.00 | 0.00 | C |
| ATOM | 3438 | O | ALA | A | 383 | 15.983 | 66.691 | −0.530 | 1.00 | 0.00 | O |
| ATOM | 3439 | CB | ALA | A | 383 | 15.947 | 63.664 | −1.423 | 1.00 | 0.00 | C |
| ATOM | 3440 | H | ALA | A | 383 | 16.346 | 62.722 | 0.891 | 1.00 | 0.00 | H |
| ATOM | 3441 | HA | ALA | A | 383 | 17.633 | 64.567 | −0.914 | 1.00 | 0.00 | H |
| ATOM | 3442 | 1HB | ALA | A | 383 | 15.740 | 64.337 | −2.255 | 1.00 | 0.00 | H |
| ATOM | 3443 | 2HB | ALA | A | 383 | 16.542 | 62.822 | −1.777 | 1.00 | 0.00 | H |
| ATOM | 3444 | 3HB | ALA | A | 383 | 15.007 | 63.296 | −1.011 | 1.00 | 0.00 | H |
| ATOM | 3445 | N | THR | A | 384 | 15.660 | 65.667 | 1.462 | 1.00 | 0.00 | N |
| ATOM | 3446 | CA | THR | A | 384 | 14.938 | 66.706 | 2.179 | 1.00 | 0.00 | C |
| ATOM | 3447 | C | THR | A | 384 | 15.766 | 67.106 | 3.415 | 1.00 | 0.00 | C |
| ATOM | 3448 | O | THR | A | 384 | 16.785 | 66.469 | 3.709 | 1.00 | 0.00 | O |
| ATOM | 3449 | CB | THR | A | 384 | 13.602 | 66.087 | 2.623 | 1.00 | 0.00 | C |
| ATOM | 3450 | CG2 | THR | A | 384 | 12.809 | 65.492 | 1.465 | 1.00 | 0.00 | C |
| ATOM | 3451 | OG1 | THR | A | 384 | 13.833 | 65.027 | 3.529 | 1.00 | 0.00 | O |
| ATOM | 3452 | H | THR | A | 384 | 15.857 | 64.859 | 2.035 | 1.00 | 0.00 | H |
| ATOM | 3453 | HA | THR | A | 384 | 14.828 | 67.581 | 1.538 | 1.00 | 0.00 | H |
| ATOM | 3454 | HB | THR | A | 384 | 12.973 | 66.858 | 3.066 | 1.00 | 0.00 | H |
| ATOM | 3455 | 1HG2 | THR | A | 384 | 11.876 | 65.070 | 1.842 | 1.00 | 0.00 | H |
| ATOM | 3456 | 2HG2 | THR | A | 384 | 12.586 | 66.272 | 0.737 | 1.00 | 0.00 | H |
| ATOM | 3457 | 3HG2 | THR | A | 384 | 13.395 | 64.706 | 0.988 | 1.00 | 0.00 | H |
| ATOM | 3458 | HG1 | THR | A | 384 | 12.994 | 64.647 | 3.799 | 1.00 | 0.00 | H |
| ATOM | 3459 | N | PHE | A | 385 | 15.334 | 68.102 | 4.190 | 1.00 | 0.00 | N |
| ATOM | 3460 | CA | PHE | A | 385 | 15.855 | 68.358 | 5.524 | 1.00 | 0.00 | C |
| ATOM | 3461 | C | PHE | A | 385 | 14.726 | 68.811 | 6.462 | 1.00 | 0.00 | C |
| ATOM | 3462 | O | PHE | A | 385 | 14.681 | 69.972 | 6.872 | 1.00 | 0.00 | O |
| ATOM | 3463 | CB | PHE | A | 385 | 16.986 | 69.393 | 5.441 | 1.00 | 0.00 | C |
| ATOM | 3464 | CG | PHE | A | 385 | 17.707 | 69.645 | 6.753 | 1.00 | 0.00 | C |
| ATOM | 3465 | CD1 | PHE | A | 385 | 18.547 | 68.661 | 7.306 | 1.00 | 0.00 | C |
| ATOM | 3466 | CD2 | PHE | A | 385 | 17.544 | 70.864 | 7.440 | 1.00 | 0.00 | C |
| ATOM | 3467 | CE1 | PHE | A | 385 | 19.213 | 68.889 | 8.523 | 1.00 | 0.00 | C |
| ATOM | 3468 | CE2 | PHE | A | 385 | 18.218 | 71.103 | 8.650 | 1.00 | 0.00 | C |
| ATOM | 3469 | CZ | PHE | A | 385 | 19.055 | 70.114 | 9.192 | 1.00 | 0.00 | C |
| ATOM | 3470 | H | PHE | A | 385 | 14.611 | 68.723 | 3.857 | 1.00 | 0.00 | H |
| ATOM | 3471 | HA | PHE | A | 385 | 16.362 | 67.467 | 5.895 | 1.00 | 0.00 | H |
| ATOM | 3472 | 1HB | PHE | A | 385 | 17.737 | 69.054 | 4.727 | 1.00 | 0.00 | H |
| ATOM | 3473 | 2HB | PHE | A | 385 | 16.580 | 70.350 | 5.114 | 1.00 | 0.00 | H |
| ATOM | 3474 | HD1 | PHE | A | 385 | 18.686 | 67.720 | 6.794 | 1.00 | 0.00 | H |
| ATOM | 3475 | HD2 | PHE | A | 385 | 16.895 | 71.628 | 7.039 | 1.00 | 0.00 | H |
| ATOM | 3476 | HE1 | PHE | A | 385 | 19.846 | 68.123 | 8.944 | 1.00 | 0.00 | H |
| ATOM | 3477 | HE2 | PHE | A | 385 | 18.091 | 72.046 | 9.161 | 1.00 | 0.00 | H |
| ATOM | 3478 | HZ | PHE | A | 385 | 19.577 | 70.293 | 10.121 | 1.00 | 0.00 | H |
| ATOM | 3479 | N | PRO | A | 386 | 13.856 | 67.886 | 6.888 | 1.00 | 0.00 | N |
| ATOM | 3480 | CA | PRO | A | 386 | 12.648 | 68.274 | 7.589 | 1.00 | 0.00 | C |
| ATOM | 3481 | C | PRO | A | 386 | 12.948 | 68.330 | 9.086 | 1.00 | 0.00 | C |
| ATOM | 3482 | O | PRO | A | 386 | 14.035 | 67.959 | 9.544 | 1.00 | 0.00 | O |
| ATOM | 3483 | CB | PRO | A | 386 | 11.613 | 67.189 | 7.266 | 1.00 | 0.00 | C |
| ATOM | 3484 | CG | PRO | A | 386 | 12.473 | 65.962 | 6.971 | 1.00 | 0.00 | C |
| ATOM | 3485 | CD | PRO | A | 386 | 13.642 | 66.610 | 6.236 | 1.00 | 0.00 | C |
| ATOM | 3486 | HA | PRO | A | 386 | 12.326 | 69.256 | 7.243 | 1.00 | 0.00 | H |
| ATOM | 3487 | 1HB | PRO | A | 386 | 10.960 | 67.042 | 8.126 | 1.00 | 0.00 | H |
| ATOM | 3488 | 2HB | PRO | A | 386 | 11.018 | 67.498 | 6.407 | 1.00 | 0.00 | H |
| ATOM | 3489 | 1HG | PRO | A | 386 | 12.756 | 65.482 | 7.907 | 1.00 | 0.00 | H |
| ATOM | 3490 | 2HG | PRO | A | 386 | 11.907 | 65.260 | 6.360 | 1.00 | 0.00 | H |
| ATOM | 3491 | 1HD | PRO | A | 386 | 13.408 | 66.774 | 5.184 | 1.00 | 0.00 | H |
| ATOM | 3492 | 2HD | PRO | A | 386 | 14.545 | 66.004 | 6.316 | 1.00 | 0.00 | H |
| ATOM | 3493 | N | LYS | A | 387 | 11.971 | 68.763 | 9.886 | 1.00 | 0.00 | N |
| ATOM | 3494 | CA | LYS | A | 387 | 12.196 | 68.920 | 11.311 | 1.00 | 0.00 | C |
| ATOM | 3495 | C | LYS | A | 387 | 12.503 | 67.568 | 11.975 | 1.00 | 0.00 | C |
| ATOM | 3496 | O | LYS | A | 387 | 13.006 | 67.541 | 13.096 | 1.00 | 0.00 | O |
| ATOM | 3497 | CB | LYS | A | 387 | 11.053 | 69.720 | 11.966 | 1.00 | 0.00 | C |
| ATOM | 3498 | CG | LYS | A | 387 | 9.826 | 68.880 | 12.358 | 1.00 | 0.00 | C |
| ATOM | 3499 | CD | LYS | A | 387 | 9.845 | 68.463 | 13.846 | 1.00 | 0.00 | C |
| ATOM | 3500 | CE | LYS | A | 387 | 9.678 | 66.953 | 14.075 | 1.00 | 0.00 | C |
| ATOM | 3501 | NZ | LYS | A | 387 | 8.469 | 66.391 | 13.448 | 1.00 | 0.00 | N1+ |
| ATOM | 3502 | H | LYS | A | 387 | 11.064 | 68.984 | 9.501 | 1.00 | 0.00 | H |
| ATOM | 3503 | HA | LYS | A | 387 | 12.930 | 69.709 | 11.478 | 1.00 | 0.00 | H |
| ATOM | 3504 | 1HB | LYS | A | 387 | 11.417 | 70.192 | 12.879 | 1.00 | 0.00 | H |
| ATOM | 3505 | 2HB | LYS | A | 387 | 10.703 | 70.487 | 11.276 | 1.00 | 0.00 | H |
| ATOM | 3506 | 1HG | LYS | A | 387 | 8.919 | 69.459 | 12.183 | 1.00 | 0.00 | H |
| ATOM | 3507 | 2HG | LYS | A | 387 | 9.799 | 67.973 | 11.755 | 1.00 | 0.00 | H |
| ATOM | 3508 | 1HD | LYS | A | 387 | 10.796 | 68.752 | 14.294 | 1.00 | 0.00 | H |
| ATOM | 3509 | 2HD | LYS | A | 387 | 9.030 | 68.959 | 14.374 | 1.00 | 0.00 | H |
| ATOM | 3510 | 1HE | LYS | A | 387 | 10.536 | 66.425 | 13.658 | 1.00 | 0.00 | H |
| ATOM | 3511 | 2HE | LYS | A | 387 | 9.613 | 66.752 | 15.145 | 1.00 | 0.00 | H |
| ATOM | 3512 | 1HZ | LYS | A | 387 | 8.421 | 65.400 | 13.637 | 1.00 | 0.00 | H |
| ATOM | 3513 | 2HZ | LYS | A | 387 | 7.651 | 66.846 | 13.828 | 1.00 | 0.00 | H |
| ATOM | 3514 | 3HZ | LYS | A | 387 | 8.506 | 66.543 | 12.450 | 1.00 | 0.00 | H |
| ATOM | 3515 | N | GLU | A | 388 | 12.184 | 66.457 | 11.300 | 1.00 | 0.00 | N |
| ATOM | 3516 | CA | GLU | A | 388 | 12.551 | 65.116 | 11.722 | 1.00 | 0.00 | C |
| ATOM | 3517 | C | GLU | A | 388 | 14.078 | 65.006 | 11.701 | 1.00 | 0.00 | C |
| ATOM | 3518 | O | GLU | A | 388 | 14.717 | 64.791 | 12.728 | 1.00 | 0.00 | O |
| ATOM | 3519 | CB | GLU | A | 388 | 11.924 | 64.029 | 10.821 | 1.00 | 0.00 | C |
| ATOM | 3520 | CG | GLU | A | 388 | 10.557 | 64.363 | 10.209 | 1.00 | 0.00 | C |
| ATOM | 3521 | CD | GLU | A | 388 | 9.578 | 64.881 | 11.234 | 1.00 | 0.00 | C |
| ATOM | 3522 | OE1 | GLU | A | 388 | 9.509 | 64.317 | 12.348 | 1.00 | 0.00 | O |
| ATOM | 3523 | OE2 | GLU | A | 388 | 8.955 | 65.931 | 10.972 | 1.00 | 0.00 | O1− |
| ATOM | 3524 | H | GLU | A | 388 | 11.656 | 66.522 | 10.441 | 1.00 | 0.00 | H |
| ATOM | 3525 | HA | GLU | A | 388 | 12.149 | 64.924 | 12.717 | 1.00 | 0.00 | H |
| ATOM | 3526 | 1HB | GLU | A | 388 | 12.587 | 63.825 | 9.981 | 1.00 | 0.00 | H |
| ATOM | 3527 | 2HB | GLU | A | 388 | 11.779 | 63.116 | 11.399 | 1.00 | 0.00 | H |
| ATOM | 3528 | 1HG | GLU | A | 388 | 10.678 | 65.129 | 9.443 | 1.00 | 0.00 | H |
| ATOM | 3529 | 2HG | GLU | A | 388 | 10.130 | 63.465 | 9.761 | 1.00 | 0.00 | H |
| ATOM | 3530 | N | ILE | A | 389 | 14.668 | 65.151 | 10.513 | 1.00 | 0.00 | N |
| ATOM | 3531 | CA | ILE | A | 389 | 16.100 | 65.020 | 10.300 | 1.00 | 0.00 | C |
| ATOM | 3532 | C | ILE | A | 389 | 16.826 | 66.077 | 11.137 | 1.00 | 0.00 | C |
| ATOM | 3533 | O | ILE | A | 389 | 17.913 | 65.836 | 11.663 | 1.00 | 0.00 | O |
| ATOM | 3534 | CB | ILE | A | 389 | 16.414 | 65.150 | 8.795 | 1.00 | 0.00 | C |
| ATOM | 3535 | CG1 | ILE | A | 389 | 15.814 | 64.004 | 7.954 | 1.00 | 0.00 | C |
| ATOM | 3536 | CG2 | ILE | A | 389 | 17.920 | 65.248 | 8.535 | 1.00 | 0.00 | C |
| ATOM | 3537 | CD1 | ILE | A | 389 | 16.494 | 62.643 | 8.143 | 1.00 | 0.00 | C |
| ATOM | 3538 | H | ILE | A | 389 | 14.113 | 65.363 | 9.696 | 1.00 | 0.00 | H |
| ATOM | 3539 | HA | ILE | A | 389 | 16.414 | 64.007 | 10.551 | 1.00 | 0.00 | H |
| ATOM | 3540 | HB | ILE | A | 389 | 16.073 | 66.121 | 8.434 | 1.00 | 0.00 | H |
| ATOM | 3541 | 1HG1 | ILE | A | 389 | 14.765 | 63.870 | 8.219 | 1.00 | 0.00 | H |
| ATOM | 3542 | 2HG1 | ILE | A | 389 | 15.892 | 64.250 | 6.895 | 1.00 | 0.00 | H |
| ATOM | 3543 | 1HG2 | ILE | A | 389 | 18.100 | 65.338 | 7.463 | 1.00 | 0.00 | H |
| ATOM | 3544 | 2HG2 | ILE | A | 389 | 18.321 | 66.124 | 9.045 | 1.00 | 0.00 | H |
| ATOM | 3545 | 3HG2 | ILE | A | 389 | 18.414 | 64.352 | 8.911 | 1.00 | 0.00 | H |
| ATOM | 3546 | 1HD1 | ILE | A | 389 | 16.004 | 61.901 | 7.513 | 1.00 | 0.00 | H |
| ATOM | 3547 | 2HD1 | ILE | A | 389 | 17.545 | 62.719 | 7.864 | 1.00 | 0.00 | H |
| ATOM | 3548 | 3HD1 | ILE | A | 389 | 16.417 | 62.340 | 9.187 | 1.00 | 0.00 | H |
| ATOM | 3549 | N | GLN | A | 390 | 16.225 | 67.259 | 11.256 | 1.00 | 0.00 | N |
| ATOM | 3550 | CA | GLN | A | 390 | 16.803 | 68.366 | 11.989 | 1.00 | 0.00 | C |
| ATOM | 3551 | C | GLN | A | 390 | 16.964 | 68.025 | 13.479 | 1.00 | 0.00 | C |
| ATOM | 3552 | O | GLN | A | 390 | 17.757 | 68.667 | 14.175 | 1.00 | 0.00 | O |
| ATOM | 3553 | CB | GLN | A | 390 | 15.929 | 69.603 | 11.771 | 1.00 | 0.00 | C |
| ATOM | 3554 | CG | GLN | A | 390 | 16.664 | 70.917 | 12.044 | 1.00 | 0.00 | C |
| ATOM | 3555 | CD | GLN | A | 390 | 15.806 | 72.113 | 11.654 | 1.00 | 0.00 | C |
| ATOM | 3556 | NE2 | GLN | A | 390 | 15.407 | 72.207 | 10.392 | 1.00 | 0.00 | N |
| ATOM | 3557 | OE1 | GLN | A | 390 | 15.503 | 72.956 | 12.490 | 1.00 | 0.00 | O |
| ATOM | 3558 | H | GLN | A | 390 | 15.324 | 67.414 | 10.825 | 1.00 | 0.00 | H |
| ATOM | 3559 | HA | GLN | A | 390 | 17.754 | 68.645 | 11.536 | 1.00 | 0.00 | H |
| ATOM | 3560 | 1HB | GLN | A | 390 | 15.585 | 69.628 | 10.737 | 1.00 | 0.00 | H |
| ATOM | 3561 | 2HB | GLN | A | 390 | 15.068 | 69.563 | 12.439 | 1.00 | 0.00 | H |
| ATOM | 3562 | 1HG | GLN | A | 390 | 16.901 | 70.988 | 13.105 | 1.00 | 0.00 | H |
| ATOM | 3563 | 2HG | GLN | A | 390 | 17.586 | 70.945 | 11.464 | 1.00 | 0.00 | H |
| ATOM | 3564 | 1HE2 | GLN | A | 390 | 14.837 | 72.987 | 10.099 | 1.00 | 0.00 | H |
| ATOM | 3565 | 2HE2 | GLN | A | 390 | 15.673 | 71.497 | 9.724 | 1.00 | 0.00 | H |
| ATOM | 3566 | N | MET | A | 391 | 16.217 | 67.032 | 13.966 | 1.00 | 0.00 | N |
| ATOM | 3567 | CA | MET | A | 391 | 16.315 | 66.505 | 15.316 | 1.00 | 0.00 | C |
| ATOM | 3568 | C | MET | A | 391 | 17.173 | 65.243 | 15.287 | 1.00 | 0.00 | C |
| ATOM | 3569 | O | MET | A | 391 | 18.144 | 65.148 | 16.027 | 1.00 | 0.00 | O |
| ATOM | 3570 | CB | MET | A | 391 | 14.919 | 66.213 | 15.881 | 1.00 | 0.00 | C |
| ATOM | 3571 | CG | MET | A | 391 | 14.157 | 67.511 | 16.167 | 1.00 | 0.00 | C |
| ATOM | 3572 | SD | MET | A | 391 | 12.416 | 67.299 | 16.634 | 1.00 | 0.00 | S |
| ATOM | 3573 | CE | MET | A | 391 | 12.585 | 66.311 | 18.144 | 1.00 | 0.00 | C |
| ATOM | 3574 | H | MET | A | 391 | 15.522 | 66.589 | 13.382 | 1.00 | 0.00 | H |
| ATOM | 3575 | HA | MET | A | 391 | 16.738 | 67.264 | 15.973 | 1.00 | 0.00 | H |
| ATOM | 3576 | 1HB | MET | A | 391 | 14.350 | 65.627 | 15.159 | 1.00 | 0.00 | H |
| ATOM | 3577 | 2HB | MET | A | 391 | 15.013 | 65.652 | 16.810 | 1.00 | 0.00 | H |
| ATOM | 3578 | 1HG | MET | A | 391 | 14.635 | 68.040 | 16.991 | 1.00 | 0.00 | H |
| ATOM | 3579 | 2HG | MET | A | 391 | 14.164 | 68.141 | 15.278 | 1.00 | 0.00 | H |
| ATOM | 3580 | 1HE | MET | A | 391 | 11.597 | 66.098 | 18.552 | 1.00 | 0.00 | H |
| ATOM | 3581 | 2HE | MET | A | 391 | 13.091 | 65.375 | 17.911 | 1.00 | 0.00 | H |
| ATOM | 3582 | 3HE | MET | A | 391 | 13.169 | 66.866 | 18.878 | 1.00 | 0.00 | H |
| ATOM | 3583 | N | LEU | A | 392 | 16.840 | 64.290 | 14.420 | 1.00 | 0.00 | N |
| ATOM | 3584 | CA | LEU | A | 392 | 17.452 | 62.966 | 14.391 | 1.00 | 0.00 | C |
| ATOM | 3585 | C | LEU | A | 392 | 18.960 | 63.057 | 14.109 | 1.00 | 0.00 | C |
| ATOM | 3586 | O | LEU | A | 392 | 19.726 | 62.139 | 14.396 | 1.00 | 0.00 | O |
| ATOM | 3587 | CB | LEU | A | 392 | 16.717 | 62.135 | 13.328 | 1.00 | 0.00 | C |
| ATOM | 3588 | CG | LEU | A | 392 | 16.990 | 60.623 | 13.355 | 1.00 | 0.00 | C |
| ATOM | 3589 | CD1 | LEU | A | 392 | 16.451 | 59.978 | 14.635 | 1.00 | 0.00 | C |
| ATOM | 3590 | CD2 | LEU | A | 392 | 16.282 | 59.989 | 12.152 | 1.00 | 0.00 | C |
| ATOM | 3591 | H | LEU | A | 392 | 16.125 | 64.463 | 13.729 | 1.00 | 0.00 | H |
| ATOM | 3592 | HA | LEU | A | 392 | 17.261 | 62.456 | 15.335 | 1.00 | 0.00 | H |
| ATOM | 3593 | 1HB | LEU | A | 392 | 15.641 | 62.252 | 13.454 | 1.00 | 0.00 | H |
| ATOM | 3594 | 2HB | LEU | A | 392 | 17.006 | 62.478 | 12.334 | 1.00 | 0.00 | H |
| ATOM | 3595 | HG | LEU | A | 392 | 18.065 | 60.447 | 13.317 | 1.00 | 0.00 | H |
| ATOM | 3596 | 1HD1 | LEU | A | 392 | 16.661 | 58.909 | 14.620 | 1.00 | 0.00 | H |
| ATOM | 3597 | 2HD1 | LEU | A | 392 | 16.933 | 60.431 | 15.501 | 1.00 | 0.00 | H |
| ATOM | 3598 | 3HD1 | LEU | A | 392 | 15.374 | 60.136 | 14.696 | 1.00 | 0.00 | H |
| ATOM | 3599 | 1HD2 | LEU | A | 392 | 16.462 | 58.914 | 12.149 | 1.00 | 0.00 | H |
| ATOM | 3600 | 2HD2 | LEU | A | 392 | 15.211 | 60.178 | 12.218 | 1.00 | 0.00 | H |
| ATOM | 3601 | 3HD2 | LEU | A | 392 | 16.671 | 60.424 | 11.230 | 1.00 | 0.00 | H |
| ATOM | 3602 | N | ALA | A | 393 | 19.409 | 64.179 | 13.544 | 1.00 | 0.00 | N |
| ATOM | 3603 | CA | ALA | A | 393 | 20.817 | 64.449 | 13.315 | 1.00 | 0.00 | C |
| ATOM | 3604 | C | ALA | A | 393 | 21.501 | 64.980 | 14.586 | 1.00 | 0.00 | C |
| ATOM | 3605 | O | ALA | A | 393 | 22.583 | 65.568 | 14.510 | 1.00 | 0.00 | O |
| ATOM | 3606 | CB | ALA | A | 393 | 20.946 | 65.436 | 12.149 | 1.00 | 0.00 | C |
| ATOM | 3607 | H | ALA | A | 393 | 18.758 | 64.893 | 13.252 | 1.00 | 0.00 | H |
| ATOM | 3608 | HA | ALA | A | 393 | 21.306 | 63.546 | 12.950 | 1.00 | 0.00 | H |
| ATOM | 3609 | 1HB | ALA | A | 393 | 21.999 | 65.647 | 11.966 | 1.00 | 0.00 | H |
| ATOM | 3610 | 2HB | ALA | A | 393 | 20.503 | 65.000 | 11.254 | 1.00 | 0.00 | H |
| ATOM | 3611 | 3HB | ALA | A | 393 | 20.428 | 66.362 | 12.397 | 1.00 | 0.00 | H |
| ATOM | 3612 | N | ARG | A | 394 | 20.858 | 64.875 | 15.752 | 1.00 | 0.00 | N |
| ATOM | 3613 | CA | ARG | A | 394 | 21.453 | 65.023 | 17.076 | 1.00 | 0.00 | C |
| ATOM | 3614 | C | ARG | A | 394 | 21.645 | 63.611 | 17.626 | 1.00 | 0.00 | C |
| ATOM | 3615 | O | ARG | A | 394 | 22.711 | 63.278 | 18.143 | 1.00 | 0.00 | O |
| ATOM | 3616 | CB | ARG | A | 394 | 20.543 | 65.814 | 18.038 | 1.00 | 0.00 | C |
| ATOM | 3617 | CG | ARG | A | 394 | 20.532 | 67.350 | 17.926 | 1.00 | 0.00 | C |
| ATOM | 3618 | CD | ARG | A | 394 | 20.071 | 67.924 | 16.580 | 1.00 | 0.00 | C |
| ATOM | 3619 | NE | ARG | A | 394 | 21.122 | 67.766 | 15.572 | 1.00 | 0.00 | N |
| ATOM | 3620 | CZ | ARG | A | 394 | 21.256 | 68.432 | 14.426 | 1.00 | 0.00 | C |
| ATOM | 3621 | NH1 | ARG | A | 394 | 20.349 | 69.329 | 14.046 | 1.00 | 0.00 | N1+ |
| ATOM | 3622 | NH2 | ARG | A | 394 | 22.323 | 68.169 | 13.682 | 1.00 | 0.00 | N |
| ATOM | 3623 | H | ARG | A | 394 | 19.868 | 64.677 | 15.766 | 1.00 | 0.00 | H |
| ATOM | 3624 | HA | ARG | A | 394 | 22.416 | 65.525 | 16.988 | 1.00 | 0.00 | H |
| ATOM | 3625 | 1HB | ARG | A | 394 | 19.506 | 65.513 | 17.889 | 1.00 | 0.00 | H |
| ATOM | 3626 | 2HB | ARG | A | 394 | 20.836 | 65.608 | 19.068 | 1.00 | 0.00 | H |
| ATOM | 3627 | 1HG | ARG | A | 394 | 19.860 | 67.765 | 18.678 | 1.00 | 0.00 | H |
| ATOM | 3628 | 2HG | ARG | A | 394 | 21.539 | 67.734 | 18.088 | 1.00 | 0.00 | H |
| ATOM | 3629 | 1HD | ARG | A | 394 | 19.177 | 67.396 | 16.247 | 1.00 | 0.00 | H |
| ATOM | 3630 | 2HD | ARG | A | 394 | 19.845 | 68.984 | 16.695 | 1.00 | 0.00 | H |
| ATOM | 3631 | HE | ARG | A | 394 | 21.795 | 67.057 | 15.826 | 1.00 | 0.00 | H |
| ATOM | 3632 | 1HH1 | ARG | A | 394 | 20.465 | 69.826 | 13.174 | 1.00 | 0.00 | H |
| ATOM | 3633 | 2HH1 | ARG | A | 394 | 19.545 | 69.512 | 14.629 | 1.00 | 0.00 | H |
| ATOM | 3634 | 1HH2 | ARG | A | 394 | 22.460 | 68.653 | 12.806 | 1.00 | 0.00 | H |
| ATOM | 3635 | 2HH2 | ARG | A | 394 | 22.997 | 67.484 | 13.991 | 1.00 | 0.00 | H |
| ATOM | 3636 | N | ASP | A | 395 | 20.602 | 62.789 | 17.527 | 1.00 | 0.00 | N |
| ATOM | 3637 | CA | ASP | A | 395 | 20.645 | 61.390 | 17.897 | 1.00 | 0.00 | C |
| ATOM | 3638 | C | ASP | A | 395 | 21.814 | 60.736 | 17.172 | 1.00 | 0.00 | C |
| ATOM | 3639 | O | ASP | A | 395 | 22.777 | 60.280 | 17.786 | 1.00 | 0.00 | O |
| ATOM | 3640 | CB | ASP | A | 395 | 19.349 | 60.673 | 17.499 | 1.00 | 0.00 | C |
| ATOM | 3641 | CG | ASP | A | 395 | 18.129 | 61.176 | 18.237 | 1.00 | 0.00 | C |
| ATOM | 3642 | OD1 | ASP | A | 395 | 17.856 | 62.388 | 18.130 | 1.00 | 0.00 | O |
| ATOM | 3643 | OD2 | ASP | A | 395 | 17.456 | 60.321 | 18.854 | 1.00 | 0.00 | O1− |
| ATOM | 3644 | H | ASP | A | 395 | 19.718 | 63.134 | 17.179 | 1.00 | 0.00 | H |
| ATOM | 3645 | HA | ASP | A | 395 | 20.785 | 61.302 | 18.974 | 1.00 | 0.00 | H |
| ATOM | 3646 | 1HB | ASP | A | 395 | 19.166 | 60.814 | 16.434 | 1.00 | 0.00 | H |
| ATOM | 3647 | 2HB | ASP | A | 395 | 19.442 | 59.608 | 17.713 | 1.00 | 0.00 | H |
| ATOM | 3648 | N | PHE | A | 396 | 21.712 | 60.665 | 15.847 | 1.00 | 0.00 | N |
| ATOM | 3649 | CA | PHE | A | 396 | 22.493 | 59.724 | 15.076 | 1.00 | 0.00 | C |
| ATOM | 3650 | C | PHE | A | 396 | 23.944 | 60.156 | 14.903 | 1.00 | 0.00 | C |
| ATOM | 3651 | O | PHE | A | 396 | 24.763 | 59.303 | 14.564 | 1.00 | 0.00 | O |
| ATOM | 3652 | CB | PHE | A | 396 | 21.840 | 59.492 | 13.707 | 1.00 | 0.00 | C |
| ATOM | 3653 | CG | PHE | A | 396 | 20.611 | 58.602 | 13.660 | 1.00 | 0.00 | C |
| ATOM | 3654 | CD1 | PHE | A | 396 | 20.070 | 57.999 | 14.815 | 1.00 | 0.00 | C |
| ATOM | 3655 | CD2 | PHE | A | 396 | 19.987 | 58.360 | 12.420 | 1.00 | 0.00 | C |
| ATOM | 3656 | CE1 | PHE | A | 396 | 18.934 | 57.184 | 14.726 | 1.00 | 0.00 | C |
| ATOM | 3657 | CE2 | PHE | A | 396 | 18.840 | 57.551 | 12.333 | 1.00 | 0.00 | C |
| ATOM | 3658 | CZ | PHE | A | 396 | 18.305 | 56.968 | 13.492 | 1.00 | 0.00 | C |
| ATOM | 3659 | H | PHE | A | 396 | 21.076 | 61.278 | 15.356 | 1.00 | 0.00 | H |
| ATOM | 3660 | HA | PHE | A | 396 | 22.438 | 58.739 | 15.539 | 1.00 | 0.00 | H |
| ATOM | 3661 | 1HB | PHE | A | 396 | 21.521 | 60.447 | 13.290 | 1.00 | 0.00 | H |
| ATOM | 3662 | 2HB | PHE | A | 396 | 22.560 | 59.024 | 13.035 | 1.00 | 0.00 | H |
| ATOM | 3663 | HD1 | PHE | A | 396 | 20.532 | 58.163 | 15.777 | 1.00 | 0.00 | H |
| ATOM | 3664 | HD2 | PHE | A | 396 | 20.389 | 58.798 | 11.518 | 1.00 | 0.00 | H |
| ATOM | 3665 | HE1 | PHE | A | 396 | 18.535 | 56.716 | 15.613 | 1.00 | 0.00 | H |
| ATOM | 3666 | HE2 | PHE | A | 396 | 18.371 | 57.380 | 11.375 | 1.00 | 0.00 | H |
| ATOM | 3667 | HZ | PHE | A | 396 | 17.417 | 56.357 | 13.439 | 1.00 | 0.00 | H |
| ATOM | 3668 | N | LEU | A | 397 | 24.257 | 61.436 | 15.106 | 1.00 | 0.00 | N |
| ATOM | 3669 | CA | LEU | A | 397 | 25.547 | 62.032 | 14.763 | 1.00 | 0.00 | C |
| ATOM | 3670 | C | LEU | A | 397 | 26.107 | 62.747 | 15.994 | 1.00 | 0.00 | C |
| ATOM | 3671 | O | LEU | A | 397 | 25.330 | 63.184 | 16.841 | 1.00 | 0.00 | O |
| ATOM | 3672 | CB | LEU | A | 397 | 25.373 | 63.074 | 13.643 | 1.00 | 0.00 | C |
| ATOM | 3673 | CG | LEU | A | 397 | 24.502 | 62.659 | 12.446 | 1.00 | 0.00 | C |
| ATOM | 3674 | CD1 | LEU | A | 397 | 24.434 | 63.823 | 11.456 | 1.00 | 0.00 | C |
| ATOM | 3675 | CD2 | LEU | A | 397 | 25.042 | 61.438 | 11.700 | 1.00 | 0.00 | C |
| ATOM | 3676 | H | LEU | A | 397 | 23.582 | 62.061 | 15.522 | 1.00 | 0.00 | H |
| ATOM | 3677 | HA | LEU | A | 397 | 26.245 | 61.248 | 14.471 | 1.00 | 0.00 | H |
| ATOM | 3678 | 1HB | LEU | A | 397 | 24.909 | 63.972 | 14.050 | 1.00 | 0.00 | H |
| ATOM | 3679 | 2HB | LEU | A | 397 | 26.348 | 63.327 | 13.227 | 1.00 | 0.00 | H |
| ATOM | 3680 | HG | LEU | A | 397 | 23.499 | 62.412 | 12.794 | 1.00 | 0.00 | H |
| ATOM | 3681 | 1HD1 | LEU | A | 397 | 23.819 | 63.539 | 10.602 | 1.00 | 0.00 | H |
| ATOM | 3682 | 2HD1 | LEU | A | 397 | 23.997 | 64.693 | 11.946 | 1.00 | 0.00 | H |
| ATOM | 3683 | 3HD1 | LEU | A | 397 | 25.440 | 64.067 | 11.113 | 1.00 | 0.00 | H |
| ATOM | 3684 | 1HD2 | LEU | A | 397 | 24.381 | 61.198 | 10.867 | 1.00 | 0.00 | H |
| ATOM | 3685 | 2HD2 | LEU | A | 397 | 26.040 | 61.657 | 11.320 | 1.00 | 0.00 | H |
| ATOM | 3686 | 3HD2 | LEU | A | 397 | 25.092 | 60.588 | 12.381 | 1.00 | 0.00 | H |
| ATOM | 3687 | N | ASP | A | 398 | 27.429 | 62.899 | 16.076 | 1.00 | 0.00 | N |
| ATOM | 3688 | CA | ASP | A | 398 | 28.127 | 63.704 | 17.079 | 1.00 | 0.00 | C |
| ATOM | 3689 | C | ASP | A | 398 | 29.249 | 64.429 | 16.347 | 1.00 | 0.00 | C |
| ATOM | 3690 | O | ASP | A | 398 | 29.676 | 63.957 | 15.294 | 1.00 | 0.00 | O |
| ATOM | 3691 | CB | ASP | A | 398 | 28.770 | 62.845 | 18.186 | 1.00 | 0.00 | C |
| ATOM | 3692 | CG | ASP | A | 398 | 27.887 | 62.604 | 19.395 | 1.00 | 0.00 | C |
| ATOM | 3693 | OD1 | ASP | A | 398 | 26.929 | 63.380 | 19.600 | 1.00 | 0.00 | O |
| ATOM | 3694 | OD2 | ASP | A | 398 | 28.123 | 61.590 | 20.079 | 1.00 | 0.00 | O1− |
| ATOM | 3695 | H | ASP | A | 398 | 28.033 | 62.439 | 15.410 | 1.00 | 0.00 | H |
| ATOM | 3696 | HA | ASP | A | 398 | 27.441 | 64.440 | 17.497 | 1.00 | 0.00 | H |
| ATOM | 3697 | 1HB | ASP | A | 398 | 29.026 | 61.865 | 17.784 | 1.00 | 0.00 | H |
| ATOM | 3698 | 2HB | ASP | A | 398 | 29.673 | 63.335 | 18.549 | 1.00 | 0.00 | H |
| ATOM | 3699 | N | GLU | A | 399 | 29.712 | 65.545 | 16.925 | 1.00 | 0.00 | N |
| ATOM | 3700 | CA | GLU | A | 399 | 30.709 | 66.476 | 16.400 | 1.00 | 0.00 | C |
| ATOM | 3701 | C | GLU | A | 399 | 30.763 | 66.408 | 14.866 | 1.00 | 0.00 | C |
| ATOM | 3702 | O | GLU | A | 399 | 31.700 | 65.867 | 14.275 | 1.00 | 0.00 | O |
| ATOM | 3703 | CB | GLU | A | 399 | 32.064 | 66.231 | 17.096 | 1.00 | 0.00 | C |
| ATOM | 3704 | CG | GLU | A | 399 | 32.846 | 67.529 | 17.353 | 1.00 | 0.00 | C |
| ATOM | 3705 | CD | GLU | A | 399 | 33.160 | 68.267 | 16.067 | 1.00 | 0.00 | C |
| ATOM | 3706 | OE1 | GLU | A | 399 | 32.206 | 68.755 | 15.428 | 1.00 | 0.00 | O |
| ATOM | 3707 | OE2 | GLU | A | 399 | 34.346 | 68.324 | 15.687 | 1.00 | 0.00 | O1− |
| ATOM | 3708 | H | GLU | A | 399 | 29.366 | 65.825 | 17.832 | 1.00 | 0.00 | H |
| ATOM | 3709 | HA | GLU | A | 399 | 30.524 | 67.472 | 16.802 | 1.00 | 0.00 | H |
| ATOM | 3710 | 1HB | GLU | A | 399 | 31.896 | 65.748 | 18.059 | 1.00 | 0.00 | H |
| ATOM | 3711 | 2HB | GLU | A | 399 | 32.683 | 65.588 | 16.471 | 1.00 | 0.00 | H |
| ATOM | 3712 | 1HG | GLU | A | 399 | 32.256 | 68.190 | 17.989 | 1.00 | 0.00 | H |
| ATOM | 3713 | 2HG | GLU | A | 399 | 33.788 | 67.295 | 17.849 | 1.00 | 0.00 | H |
| ATOM | 3714 | N | TYR | A | 400 | 29.721 | 66.938 | 14.229 | 1.00 | 0.00 | N |
| ATOM | 3715 | CA | TYR | A | 400 | 29.309 | 66.470 | 12.920 | 1.00 | 0.00 | C |
| ATOM | 3716 | C | TYR | A | 400 | 29.487 | 67.571 | 11.875 | 1.00 | 0.00 | C |
| ATOM | 3717 | O | TYR | A | 400 | 29.532 | 68.758 | 12.211 | 1.00 | 0.00 | O |
| ATOM | 3718 | CB | TYR | A | 400 | 27.854 | 65.969 | 13.001 | 1.00 | 0.00 | C |
| ATOM | 3719 | CG | TYR | A | 400 | 26.773 | 66.994 | 12.705 | 1.00 | 0.00 | C |
| ATOM | 3720 | CD1 | TYR | A | 400 | 26.089 | 66.956 | 11.477 | 1.00 | 0.00 | C |
| ATOM | 3721 | CD2 | TYR | A | 400 | 26.433 | 67.995 | 13.634 | 1.00 | 0.00 | C |
| ATOM | 3722 | CE1 | TYR | A | 400 | 25.091 | 67.895 | 11.182 | 1.00 | 0.00 | C |
| ATOM | 3723 | CE2 | TYR | A | 400 | 25.436 | 68.941 | 13.335 | 1.00 | 0.00 | C |
| ATOM | 3724 | CZ | TYR | A | 400 | 24.752 | 68.898 | 12.105 | 1.00 | 0.00 | C |
| ATOM | 3725 | OH | TYR | A | 400 | 23.768 | 69.804 | 11.812 | 1.00 | 0.00 | O |
| ATOM | 3726 | H | TYR | A | 400 | 29.195 | 67.687 | 14.656 | 1.00 | 0.00 | H |
| ATOM | 3727 | HA | TYR | A | 400 | 29.866 | 65.568 | 12.664 | 1.00 | 0.00 | H |
| ATOM | 3728 | 1HB | TYR | A | 400 | 27.706 | 65.162 | 12.285 | 1.00 | 0.00 | H |
| ATOM | 3729 | 2HB | TYR | A | 400 | 27.651 | 65.603 | 14.008 | 1.00 | 0.00 | H |
| ATOM | 3730 | HD1 | TYR | A | 400 | 26.330 | 66.197 | 10.748 | 1.00 | 0.00 | H |
| ATOM | 3731 | HD2 | TYR | A | 400 | 26.939 | 68.042 | 14.587 | 1.00 | 0.00 | H |
| ATOM | 3732 | HE1 | TYR | A | 400 | 24.573 | 67.853 | 10.235 | 1.00 | 0.00 | H |
| ATOM | 3733 | HE2 | TYR | A | 400 | 25.188 | 69.710 | 14.052 | 1.00 | 0.00 | H |
| ATOM | 3734 | HH | TYR | A | 400 | 23.665 | 70.414 | 12.546 | 1.00 | 0.00 | H |
| ATOM | 3735 | N | ILE | A | 401 | 29.548 | 67.186 | 10.602 | 1.00 | 0.00 | N |
| ATOM | 3736 | CA | ILE | A | 401 | 29.545 | 68.129 | 9.496 | 1.00 | 0.00 | C |
| ATOM | 3737 | C | ILE | A | 401 | 28.161 | 68.090 | 8.856 | 1.00 | 0.00 | C |
| ATOM | 3738 | O | ILE | A | 401 | 27.599 | 67.014 | 8.652 | 1.00 | 0.00 | O |
| ATOM | 3739 | CB | ILE | A | 401 | 30.644 | 67.795 | 8.472 | 1.00 | 0.00 | C |
| ATOM | 3740 | CG1 | ILE | A | 401 | 32.046 | 67.691 | 9.099 | 1.00 | 0.00 | C |
| ATOM | 3741 | CG2 | ILE | A | 401 | 30.645 | 68.842 | 7.345 | 1.00 | 0.00 | C |
| ATOM | 3742 | CD1 | ILE | A | 401 | 32.524 | 68.980 | 9.774 | 1.00 | 0.00 | C |
| ATOM | 3743 | H | ILE | A | 401 | 29.598 | 66.203 | 10.373 | 1.00 | 0.00 | H |
| ATOM | 3744 | HA | ILE | A | 401 | 29.802 | 69.122 | 9.864 | 1.00 | 0.00 | H |
| ATOM | 3745 | HB | ILE | A | 401 | 30.391 | 66.869 | 7.955 | 1.00 | 0.00 | H |
| ATOM | 3746 | 1HG1 | ILE | A | 401 | 32.048 | 66.910 | 9.860 | 1.00 | 0.00 | H |
| ATOM | 3747 | 2HG1 | ILE | A | 401 | 32.774 | 67.444 | 8.325 | 1.00 | 0.00 | H |
| ATOM | 3748 | 1HG2 | ILE | A | 401 | 31.425 | 68.600 | 6.624 | 1.00 | 0.00 | H |
| ATOM | 3749 | 2HG2 | ILE | A | 401 | 29.676 | 68.841 | 6.846 | 1.00 | 0.00 | H |
| ATOM | 3750 | 3HG2 | ILE | A | 401 | 30.834 | 69.830 | 7.767 | 1.00 | 0.00 | H |
| ATOM | 3751 | 1HD1 | ILE | A | 401 | 33.519 | 68.825 | 10.191 | 1.00 | 0.00 | H |
| ATOM | 3752 | 2HD1 | ILE | A | 401 | 32.560 | 69.784 | 9.038 | 1.00 | 0.00 | H |
| ATOM | 3753 | 3HD1 | ILE | A | 401 | 31.834 | 69.250 | 10.573 | 1.00 | 0.00 | H |
| ATOM | 3754 | N | PHE | A | 402 | 27.626 | 69.263 | 8.522 | 1.00 | 0.00 | N |
| ATOM | 3755 | CA | PHE | A | 402 | 26.339 | 69.407 | 7.880 | 1.00 | 0.00 | C |
| ATOM | 3756 | C | PHE | A | 402 | 26.573 | 69.949 | 6.477 | 1.00 | 0.00 | C |
| ATOM | 3757 | O | PHE | A | 402 | 27.143 | 71.025 | 6.323 | 1.00 | 0.00 | O |
| ATOM | 3758 | CB | PHE | A | 402 | 25.487 | 70.399 | 8.679 | 1.00 | 0.00 | C |
| ATOM | 3759 | CG | PHE | A | 402 | 24.065 | 70.617 | 8.193 | 1.00 | 0.00 | C |
| ATOM | 3760 | CD1 | PHE | A | 402 | 23.399 | 69.689 | 7.360 | 1.00 | 0.00 | C |
| ATOM | 3761 | CD2 | PHE | A | 402 | 23.388 | 71.795 | 8.565 | 1.00 | 0.00 | C |
| ATOM | 3762 | CE1 | PHE | A | 402 | 22.104 | 69.951 | 6.887 | 1.00 | 0.00 | C |
| ATOM | 3763 | CE2 | PHE | A | 402 | 22.092 | 72.057 | 8.092 | 1.00 | 0.00 | C |
| ATOM | 3764 | CZ | PHE | A | 402 | 21.454 | 71.139 | 7.246 | 1.00 | 0.00 | C |
| ATOM | 3765 | H | PHE | A | 402 | 28.126 | 70.119 | 8.716 | 1.00 | 0.00 | H |
| ATOM | 3766 | HA | PHE | A | 402 | 25.853 | 68.433 | 7.816 | 1.00 | 0.00 | H |
| ATOM | 3767 | 1HB | PHE | A | 402 | 25.400 | 70.057 | 9.710 | 1.00 | 0.00 | H |
| ATOM | 3768 | 2HB | PHE | A | 402 | 25.961 | 71.381 | 8.661 | 1.00 | 0.00 | H |
| ATOM | 3769 | HD1 | PHE | A | 402 | 23.887 | 68.767 | 7.080 | 1.00 | 0.00 | H |
| ATOM | 3770 | HD2 | PHE | A | 402 | 23.866 | 72.508 | 9.220 | 1.00 | 0.00 | H |
| ATOM | 3771 | HE1 | PHE | A | 402 | 21.608 | 69.235 | 6.248 | 1.00 | 0.00 | H |
| ATOM | 3772 | HE2 | PHE | A | 402 | 21.585 | 72.966 | 8.381 | 1.00 | 0.00 | H |
| ATOM | 3773 | HZ | PHE | A | 402 | 20.464 | 71.348 | 6.870 | 1.00 | 0.00 | H |
| ATOM | 3774 | N | LEU | A | 403 | 26.107 | 69.231 | 5.465 | 1.00 | 0.00 | N |
| ATOM | 3775 | CA | LEU | A | 403 | 26.089 | 69.676 | 4.090 | 1.00 | 0.00 | C |
| ATOM | 3776 | C | LEU | A | 403 | 24.646 | 69.585 | 3.609 | 1.00 | 0.00 | C |
| ATOM | 3777 | O | LEU | A | 403 | 23.930 | 68.651 | 3.975 | 1.00 | 0.00 | O |
| ATOM | 3778 | CB | LEU | A | 403 | 27.063 | 68.815 | 3.272 | 1.00 | 0.00 | C |
| ATOM | 3779 | CG | LEU | A | 403 | 27.109 | 69.164 | 1.773 | 1.00 | 0.00 | C |
| ATOM | 3780 | CD1 | LEU | A | 403 | 28.512 | 68.870 | 1.226 | 1.00 | 0.00 | C |
| ATOM | 3781 | CD2 | LEU | A | 403 | 26.094 | 68.350 | 0.956 | 1.00 | 0.00 | C |
| ATOM | 3782 | H | LEU | A | 403 | 25.732 | 68.308 | 5.631 | 1.00 | 0.00 | H |
| ATOM | 3783 | HA | LEU | A | 403 | 26.501 | 70.684 | 4.029 | 1.00 | 0.00 | H |
| ATOM | 3784 | 1HB | LEU | A | 403 | 28.074 | 68.940 | 3.661 | 1.00 | 0.00 | H |
| ATOM | 3785 | 2HB | LEU | A | 403 | 26.773 | 67.767 | 3.347 | 1.00 | 0.00 | H |
| ATOM | 3786 | HG | LEU | A | 403 | 26.879 | 70.221 | 1.638 | 1.00 | 0.00 | H |
| ATOM | 3787 | 1HD1 | LEU | A | 403 | 28.548 | 69.116 | 0.165 | 1.00 | 0.00 | H |
| ATOM | 3788 | 2HD1 | LEU | A | 403 | 29.246 | 69.471 | 1.764 | 1.00 | 0.00 | H |
| ATOM | 3789 | 3HD1 | LEU | A | 403 | 28.740 | 67.812 | 1.361 | 1.00 | 0.00 | H |
| ATOM | 3790 | 1HD2 | LEU | A | 403 | 26.163 | 68.631 | −0.095 | 1.00 | 0.00 | H |
| ATOM | 3791 | 2HD2 | LEU | A | 403 | 26.310 | 67.287 | 1.062 | 1.00 | 0.00 | H |
| ATOM | 3792 | 3HD2 | LEU | A | 403 | 25.087 | 68.554 | 1.320 | 1.00 | 0.00 | H |
| ATOM | 3793 | N | ALA | A | 404 | 24.219 | 70.531 | 2.777 | 1.00 | 0.00 | N |
| ATOM | 3794 | CA | ALA | A | 404 | 22.895 | 70.506 | 2.193 | 1.00 | 0.00 | C |
| ATOM | 3795 | C | ALA | A | 404 | 22.923 | 71.159 | 0.818 | 1.00 | 0.00 | C |
| ATOM | 3796 | O | ALA | A | 404 | 23.498 | 72.234 | 0.665 | 1.00 | 0.00 | O |
| ATOM | 3797 | CB | ALA | A | 404 | 21.910 | 71.254 | 3.083 | 1.00 | 0.00 | C |
| ATOM | 3798 | H | ALA | A | 404 | 24.826 | 71.301 | 2.534 | 1.00 | 0.00 | H |
| ATOM | 3799 | HA | ALA | A | 404 | 22.558 | 69.474 | 2.100 | 1.00 | 0.00 | H |
| ATOM | 3800 | 1HB | ALA | A | 404 | 20.918 | 71.227 | 2.631 | 1.00 | 0.00 | H |
| ATOM | 3801 | 2HB | ALA | A | 404 | 21.874 | 70.781 | 4.064 | 1.00 | 0.00 | H |
| ATOM | 3802 | 3HB | ALA | A | 404 | 22.231 | 72.290 | 3.191 | 1.00 | 0.00 | H |
| ATOM | 3803 | N | VAL | A | 405 | 22.256 | 70.539 | −0.152 | 1.00 | 0.00 | N |
| ATOM | 3804 | CA | VAL | A | 405 | 21.759 | 71.169 | −1.365 | 1.00 | 0.00 | C |
| ATOM | 3805 | C | VAL | A | 405 | 20.328 | 70.646 | −1.543 | 1.00 | 0.00 | C |
| ATOM | 3806 | O | VAL | A | 405 | 19.987 | 69.959 | −2.508 | 1.00 | 0.00 | O |
| ATOM | 3807 | CB | VAL | A | 405 | 22.695 | 70.912 | −2.566 | 1.00 | 0.00 | C |
| ATOM | 3808 | CG1 | VAL | A | 405 | 23.991 | 71.722 | −2.422 | 1.00 | 0.00 | C |
| ATOM | 3809 | CG2 | VAL | A | 405 | 23.071 | 69.437 | −2.770 | 1.00 | 0.00 | C |
| ATOM | 3810 | H | VAL | A | 405 | 22.059 | 69.551 | −0.077 | 1.00 | 0.00 | H |
| ATOM | 3811 | HA | VAL | A | 405 | 21.861 | 72.251 | −1.281 | 1.00 | 0.00 | H |
| ATOM | 3812 | HB | VAL | A | 405 | 22.179 | 71.168 | −3.491 | 1.00 | 0.00 | H |
| ATOM | 3813 | 1HG1 | VAL | A | 405 | 24.638 | 71.528 | −3.277 | 1.00 | 0.00 | H |
| ATOM | 3814 | 2HG1 | VAL | A | 405 | 23.753 | 72.785 | −2.380 | 1.00 | 0.00 | H |
| ATOM | 3815 | 3HG1 | VAL | A | 405 | 24.503 | 71.429 | −1.506 | 1.00 | 0.00 | H |
| ATOM | 3816 | 1HG2 | VAL | A | 405 | 23.730 | 69.345 | −3.633 | 1.00 | 0.00 | H |
| ATOM | 3817 | 2HG2 | VAL | A | 405 | 23.583 | 69.066 | −1.882 | 1.00 | 0.00 | H |
| ATOM | 3818 | 3HG2 | VAL | A | 405 | 22.167 | 68.851 | −2.939 | 1.00 | 0.00 | H |
| ATOM | 3819 | N | GLY | A | 406 | 19.507 | 70.933 | −0.536 | 1.00 | 0.00 | N |
| ATOM | 3820 | CA | GLY | A | 406 | 18.150 | 70.439 | −0.371 | 1.00 | 0.00 | C |
| ATOM | 3821 | C | GLY | A | 406 | 17.650 | 70.826 | 1.023 | 1.00 | 0.00 | C |
| ATOM | 3822 | O | GLY | A | 406 | 18.354 | 71.525 | 1.752 | 1.00 | 0.00 | O |
| ATOM | 3823 | H | GLY | A | 406 | 19.813 | 71.548 | 0.205 | 1.00 | 0.00 | H |
| ATOM | 3824 | 1HA | GLY | A | 406 | 17.506 | 70.884 | −1.130 | 1.00 | 0.00 | H |
| ATOM | 3825 | 2HA | GLY | A | 406 | 18.142 | 69.355 | −0.478 | 1.00 | 0.00 | H |
| ATOM | 3826 | HXT | GLY | A | 406 | 16.684 | 70.487 | 1.368 | 1.00 | 0.00 | H |
| ATOM | 3827 | N | THR | A | 411 | 16.873 | 68.679 | −11.615 | 1.00 | 0.00 | N1+ |
| ATOM | 3828 | CA | THR | A | 411 | 18.079 | 68.144 | −10.968 | 1.00 | 0.00 | C |
| ATOM | 3829 | C | THR | A | 411 | 18.867 | 69.321 | −10.420 | 1.00 | 0.00 | C |
| ATOM | 3830 | O | THR | A | 411 | 18.663 | 70.441 | −10.878 | 1.00 | 0.00 | O |
| ATOM | 3831 | CB | THR | A | 411 | 18.916 | 67.332 | −11.957 | 1.00 | 0.00 | C |
| ATOM | 3832 | 1H | THR | A | 411 | 16.326 | 67.918 | −11.991 | 1.00 | 0.00 | H |
| ATOM | 3833 | 2H | THR | A | 411 | 16.323 | 69.185 | −10.935 | 1.00 | 0.00 | H |
| ATOM | 3834 | 3H | THR | A | 411 | 17.140 | 69.303 | −12.363 | 1.00 | 0.00 | H |
| ATOM | 3835 | HA | THR | A | 411 | 17.791 | 67.446 | −10.182 | 1.00 | 0.00 | H |
| ATOM | 3836 | 1HB | THR | A | 411 | 19.802 | 66.947 | −11.453 | 1.00 | 0.00 | H |
| ATOM | 3837 | 2HB | THR | A | 411 | 18.324 | 66.499 | −12.336 | 1.00 | 0.00 | H |
| ATOM | 3838 | 3HB | THR | A | 411 | 19.219 | 67.970 | −12.787 | 1.00 | 0.00 | H |
| ATOM | 3839 | N | SER | A | 412 | 19.767 | 69.086 | −9.471 | 1.00 | 0.00 | N |
| ATOM | 3840 | CA | SER | A | 412 | 20.538 | 70.113 | −8.792 | 1.00 | 0.00 | C |
| ATOM | 3841 | C | SER | A | 412 | 21.360 | 70.960 | −9.774 | 1.00 | 0.00 | C |
| ATOM | 3842 | O | SER | A | 412 | 21.857 | 72.024 | −9.414 | 1.00 | 0.00 | O |
| ATOM | 3843 | CB | SER | A | 412 | 21.427 | 69.422 | −7.742 | 1.00 | 0.00 | C |
| ATOM | 3844 | OG | SER | A | 412 | 21.420 | 68.004 | −7.897 | 1.00 | 0.00 | O |
| ATOM | 3845 | H | SER | A | 412 | 19.954 | 68.138 | −9.177 | 1.00 | 0.00 | H |
| ATOM | 3846 | HA | SER | A | 412 | 19.870 | 70.737 | −8.199 | 1.00 | 0.00 | H |
| ATOM | 3847 | 1HB | SER | A | 412 | 22.454 | 69.773 | −7.845 | 1.00 | 0.00 | H |
| ATOM | 3848 | 2HB | SER | A | 412 | 21.061 | 69.659 | −6.743 | 1.00 | 0.00 | H |
| ATOM | 3849 | HG | SER | A | 412 | 21.983 | 67.606 | −7.228 | 1.00 | 0.00 | H |
| ATOM | 3850 | N | GLU | A | 413 | 21.539 | 70.456 | −10.992 | 1.00 | 0.00 | N |
| ATOM | 3851 | CA | GLU | A | 413 | 22.403 | 71.002 | −12.008 | 1.00 | 0.00 | C |
| ATOM | 3852 | C | GLU | A | 413 | 21.575 | 71.603 | −13.156 | 1.00 | 0.00 | C |
| ATOM | 3853 | O | GLU | A | 413 | 22.149 | 72.061 | −14.141 | 1.00 | 0.00 | O |
| ATOM | 3854 | CB | GLU | A | 413 | 23.292 | 69.859 | −12.538 | 1.00 | 0.00 | C |
| ATOM | 3855 | CG | GLU | A | 413 | 23.922 | 68.967 | −11.443 | 1.00 | 0.00 | C |
| ATOM | 3856 | CD | GLU | A | 413 | 22.963 | 67.960 | −10.816 | 1.00 | 0.00 | C |
| ATOM | 3857 | OE1 | GLU | A | 413 | 23.309 | 67.416 | −9.747 | 1.00 | 0.00 | O |
| ATOM | 3858 | OE2 | GLU | A | 413 | 21.864 | 67.764 | −11.374 | 1.00 | 0.00 | O1− |
| ATOM | 3859 | H | GLU | A | 413 | 21.045 | 69.619 | −11.270 | 1.00 | 0.00 | H |
| ATOM | 3860 | HA | GLU | A | 413 | 23.031 | 71.779 | −11.572 | 1.00 | 0.00 | H |
| ATOM | 3861 | 1HB | GLU | A | 413 | 22.699 | 69.203 | −13.175 | 1.00 | 0.00 | H |
| ATOM | 3862 | 2HB | GLU | A | 413 | 24.116 | 70.277 | −13.115 | 1.00 | 0.00 | H |
| ATOM | 3863 | 1HG | GLU | A | 413 | 24.746 | 68.395 | −11.869 | 1.00 | 0.00 | H |
| ATOM | 3864 | 2HG | GLU | A | 413 | 24.295 | 69.594 | −10.633 | 1.00 | 0.00 | H |
| ATOM | 3865 | N | ASN | A | 414 | 20.239 | 71.504 | −13.105 | 1.00 | 0.00 | N |
| ATOM | 3866 | CA | ASN | A | 414 | 19.373 | 71.780 | −14.254 | 1.00 | 0.00 | C |
| ATOM | 3867 | C | ASN | A | 414 | 17.903 | 71.584 | −13.863 | 1.00 | 0.00 | C |
| ATOM | 3868 | O | ASN | A | 414 | 17.521 | 70.472 | −13.493 | 1.00 | 0.00 | O |
| ATOM | 3869 | CB | ASN | A | 414 | 19.757 | 70.812 | −15.383 | 1.00 | 0.00 | C |
| ATOM | 3870 | CG | ASN | A | 414 | 18.696 | 70.619 | −16.458 | 1.00 | 0.00 | C |
| ATOM | 3871 | ND2 | ASN | A | 414 | 18.745 | 69.469 | −17.110 | 1.00 | 0.00 | N |
| ATOM | 3872 | OD1 | ASN | A | 414 | 17.852 | 71.466 | −16.726 | 1.00 | 0.00 | O |
| ATOM | 3873 | H | ASN | A | 414 | 19.785 | 71.227 | −12.247 | 1.00 | 0.00 | H |
| ATOM | 3874 | HA | ASN | A | 414 | 19.527 | 72.806 | −14.588 | 1.00 | 0.00 | H |
| ATOM | 3875 | 1HB | ASN | A | 414 | 20.649 | 71.180 | −15.890 | 1.00 | 0.00 | H |
| ATOM | 3876 | 2HB | ASN | A | 414 | 19.958 | 69.826 | −14.964 | 1.00 | 0.00 | H |
| ATOM | 3877 | 1HD2 | ASN | A | 414 | 18.072 | 69.271 | −17.837 | 1.00 | 0.00 | H |
| ATOM | 3878 | 2HD2 | ASN | A | 414 | 19.455 | 68.788 | −16.881 | 1.00 | 0.00 | H |
| ATOM | 3879 | N | ILE | A | 415 | 17.078 | 72.631 | −13.959 | 1.00 | 0.00 | N |
| ATOM | 3880 | CA | ILE | A | 415 | 15.694 | 72.657 | −13.494 | 1.00 | 0.00 | C |
| ATOM | 3881 | C | ILE | A | 415 | 14.880 | 73.536 | −14.453 | 1.00 | 0.00 | C |
| ATOM | 3882 | O | ILE | A | 415 | 15.342 | 74.613 | −14.829 | 1.00 | 0.00 | O |
| ATOM | 3883 | CB | ILE | A | 415 | 15.634 | 73.253 | −12.067 | 1.00 | 0.00 | C |
| ATOM | 3884 | CG1 | ILE | A | 415 | 16.562 | 72.516 | −11.087 | 1.00 | 0.00 | C |
| ATOM | 3885 | CG2 | ILE | A | 415 | 14.196 | 73.214 | −11.540 | 1.00 | 0.00 | C |
| ATOM | 3886 | CD1 | ILE | A | 415 | 16.486 | 73.017 | −9.641 | 1.00 | 0.00 | C |
| ATOM | 3887 | H | ILE | A | 415 | 17.401 | 73.491 | −14.381 | 1.00 | 0.00 | H |
| ATOM | 3888 | HA | ILE | A | 415 | 15.291 | 71.644 | −13.494 | 1.00 | 0.00 | H |
| ATOM | 3889 | HB | ILE | A | 415 | 15.894 | 74.311 | −12.103 | 1.00 | 0.00 | H |
| ATOM | 3890 | 1HG1 | ILE | A | 415 | 16.306 | 71.457 | −11.070 | 1.00 | 0.00 | H |
| ATOM | 3891 | 2HG1 | ILE | A | 415 | 17.597 | 72.634 | −11.409 | 1.00 | 0.00 | H |
| ATOM | 3892 | 1HG2 | ILE | A | 415 | 14.165 | 73.636 | −10.536 | 1.00 | 0.00 | H |
| ATOM | 3893 | 2HG2 | ILE | A | 415 | 13.551 | 73.797 | −12.198 | 1.00 | 0.00 | H |
| ATOM | 3894 | 3HG2 | ILE | A | 415 | 13.847 | 72.182 | −11.512 | 1.00 | 0.00 | H |
| ATOM | 3895 | 1HD1 | ILE | A | 415 | 17.174 | 72.442 | −9.021 | 1.00 | 0.00 | H |
| ATOM | 3896 | 2HD1 | ILE | A | 415 | 16.761 | 74.071 | −9.606 | 1.00 | 0.00 | H |
| ATOM | 3897 | 3HD1 | ILE | A | 415 | 15.470 | 72.894 | −9.267 | 1.00 | 0.00 | H |
| ATOM | 3898 | N | THR | A | 416 | 13.675 | 73.111 | −14.833 | 1.00 | 0.00 | N |
| ATOM | 3899 | CA | THR | A | 416 | 12.721 | 73.901 | −15.601 | 1.00 | 0.00 | C |
| ATOM | 3900 | C | THR | A | 416 | 11.307 | 73.477 | −15.157 | 1.00 | 0.00 | C |
| ATOM | 3901 | O | THR | A | 416 | 11.022 | 72.281 | −15.126 | 1.00 | 0.00 | O |
| ATOM | 3902 | CB | THR | A | 416 | 12.979 | 73.664 | −17.098 | 1.00 | 0.00 | C |
| ATOM | 3903 | CG2 | THR | A | 416 | 12.220 | 74.674 | −17.961 | 1.00 | 0.00 | C |
| ATOM | 3904 | OG1 | THR | A | 416 | 14.364 | 73.764 | −17.385 | 1.00 | 0.00 | O |
| ATOM | 3905 | H | THR | A | 416 | 13.367 | 72.181 | −14.588 | 1.00 | 0.00 | H |
| ATOM | 3906 | HA | THR | A | 416 | 12.912 | 74.962 | −15.438 | 1.00 | 0.00 | H |
| ATOM | 3907 | HB | THR | A | 416 | 12.668 | 72.654 | −17.365 | 1.00 | 0.00 | H |
| ATOM | 3908 | 1HG2 | THR | A | 416 | 12.424 | 74.478 | −19.014 | 1.00 | 0.00 | H |
| ATOM | 3909 | 2HG2 | THR | A | 416 | 11.150 | 74.580 | −17.776 | 1.00 | 0.00 | H |
| ATOM | 3910 | 3HG2 | THR | A | 416 | 12.545 | 75.684 | −17.710 | 1.00 | 0.00 | H |
| ATOM | 3911 | HG1 | THR | A | 416 | 14.509 | 73.614 | −18.322 | 1.00 | 0.00 | H |
| ATOM | 3912 | N | GLN | A | 417 | 10.433 | 74.414 | −14.777 | 1.00 | 0.00 | N |
| ATOM | 3913 | CA | GLN | A | 417 | 9.223 | 74.136 | −14.004 | 1.00 | 0.00 | C |
| ATOM | 3914 | C | GLN | A | 417 | 7.995 | 74.820 | −14.611 | 1.00 | 0.00 | C |
| ATOM | 3915 | O | GLN | A | 417 | 8.070 | 75.974 | −15.025 | 1.00 | 0.00 | O |
| ATOM | 3916 | CB | GLN | A | 417 | 9.413 | 74.643 | −12.566 | 1.00 | 0.00 | C |
| ATOM | 3917 | CG | GLN | A | 417 | 10.359 | 73.733 | −11.778 | 1.00 | 0.00 | C |
| ATOM | 3918 | CD | GLN | A | 417 | 10.778 | 74.361 | −10.455 | 1.00 | 0.00 | C |
| ATOM | 3919 | NE2 | GLN | A | 417 | 9.935 | 74.312 | −9.435 | 1.00 | 0.00 | N |
| ATOM | 3920 | OE1 | GLN | A | 417 | 11.872 | 74.897 | −10.336 | 1.00 | 0.00 | O |
| ATOM | 3921 | H | GLN | A | 417 | 10.589 | 75.380 | −15.024 | 1.00 | 0.00 | H |
| ATOM | 3922 | HA | GLN | A | 417 | 9.047 | 73.061 | −13.977 | 1.00 | 0.00 | H |
| ATOM | 3923 | 1HB | GLN | A | 417 | 9.836 | 75.647 | −12.587 | 1.00 | 0.00 | H |
| ATOM | 3924 | 2HB | GLN | A | 417 | 8.449 | 74.665 | −12.058 | 1.00 | 0.00 | H |
| ATOM | 3925 | 1HG | GLN | A | 417 | 9.860 | 72.787 | −11.564 | 1.00 | 0.00 | H |
| ATOM | 3926 | 2HG | GLN | A | 417 | 11.257 | 73.544 | −12.366 | 1.00 | 0.00 | H |
| ATOM | 3927 | 1HE2 | GLN | A | 417 | 10.191 | 74.722 | −8.548 | 1.00 | 0.00 | H |
| ATOM | 3928 | 2HE2 | GLN | A | 417 | 9.035 | 73.866 | −9.543 | 1.00 | 0.00 | H |
| ATOM | 3929 | N | LYS | A | 418 | 6.845 | 74.140 | −14.602 | 1.00 | 0.00 | N |
| ATOM | 3930 | CA | LYS | A | 418 | 5.549 | 74.715 | −14.938 | 1.00 | 0.00 | C |
| ATOM | 3931 | C | LYS | A | 418 | 4.470 | 74.063 | −14.068 | 1.00 | 0.00 | C |
| ATOM | 3932 | O | LYS | A | 418 | 4.578 | 72.883 | −13.737 | 1.00 | 0.00 | O |
| ATOM | 3933 | CB | LYS | A | 418 | 5.212 | 74.468 | −16.414 | 1.00 | 0.00 | C |
| ATOM | 3934 | CG | LYS | A | 418 | 6.108 | 75.244 | −17.387 | 1.00 | 0.00 | C |
| ATOM | 3935 | CD | LYS | A | 418 | 5.523 | 75.122 | −18.805 | 1.00 | 0.00 | C |
| ATOM | 3936 | CE | LYS | A | 418 | 6.241 | 76.046 | −19.797 | 1.00 | 0.00 | C |
| ATOM | 3937 | NZ | LYS | A | 418 | 7.656 | 75.685 | −19.975 | 1.00 | 0.00 | N1+ |
| ATOM | 3938 | H | LYS | A | 418 | 6.839 | 73.161 | −14.353 | 1.00 | 0.00 | H |
| ATOM | 3939 | HA | LYS | A | 418 | 5.567 | 75.788 | −14.748 | 1.00 | 0.00 | H |
| ATOM | 3940 | 1HB | LYS | A | 418 | 5.326 | 73.408 | −16.642 | 1.00 | 0.00 | H |
| ATOM | 3941 | 2HB | LYS | A | 418 | 4.183 | 74.772 | −16.608 | 1.00 | 0.00 | H |
| ATOM | 3942 | 1HG | LYS | A | 418 | 6.144 | 76.293 | −17.092 | 1.00 | 0.00 | H |
| ATOM | 3943 | 2HG | LYS | A | 418 | 7.115 | 74.826 | −17.366 | 1.00 | 0.00 | H |
| ATOM | 3944 | 1HD | LYS | A | 418 | 5.629 | 74.096 | −19.156 | 1.00 | 0.00 | H |
| ATOM | 3945 | 2HD | LYS | A | 418 | 4.468 | 75.393 | −18.788 | 1.00 | 0.00 | H |
| ATOM | 3946 | 1HE | LYS | A | 418 | 5.754 | 75.984 | −20.770 | 1.00 | 0.00 | H |
| ATOM | 3947 | 2HE | LYS | A | 418 | 6.199 | 77.073 | −19.434 | 1.00 | 0.00 | H |
| ATOM | 3948 | 1HZ | LYS | A | 418 | 8.086 | 76.318 | −20.634 | 1.00 | 0.00 | H |
| ATOM | 3949 | 2HZ | LYS | A | 418 | 8.135 | 75.748 | −19.087 | 1.00 | 0.00 | H |
| ATOM | 3950 | 3HZ | LYS | A | 418 | 7.722 | 74.740 | −20.325 | 1.00 | 0.00 | H |
| ATOM | 3951 | N | VAL | A | 419 | 3.415 | 74.809 | −13.731 | 1.00 | 0.00 | N |
| ATOM | 3952 | CA | VAL | A | 419 | 2.330 | 74.351 | −12.874 | 1.00 | 0.00 | C |
| ATOM | 3953 | C | VAL | A | 419 | 1.025 | 74.941 | −13.425 | 1.00 | 0.00 | C |
| ATOM | 3954 | O | VAL | A | 419 | 0.990 | 76.125 | −13.764 | 1.00 | 0.00 | O |
| ATOM | 3955 | CB | VAL | A | 419 | 2.571 | 74.803 | −11.418 | 1.00 | 0.00 | C |
| ATOM | 3956 | CG1 | VAL | A | 419 | 1.585 | 74.124 | −10.460 | 1.00 | 0.00 | C |
| ATOM | 3957 | CG2 | VAL | A | 419 | 3.993 | 74.506 | −10.915 | 1.00 | 0.00 | C |
| ATOM | 3958 | H | VAL | A | 419 | 3.335 | 75.755 | −14.074 | 1.00 | 0.00 | H |
| ATOM | 3959 | HA | VAL | A | 419 | 2.304 | 73.261 | −12.871 | 1.00 | 0.00 | H |
| ATOM | 3960 | HB | VAL | A | 419 | 2.466 | 75.886 | −11.350 | 1.00 | 0.00 | H |
| ATOM | 3961 | 1HG1 | VAL | A | 419 | 1.777 | 74.459 | −9.441 | 1.00 | 0.00 | H |
| ATOM | 3962 | 2HG1 | VAL | A | 419 | 0.565 | 74.385 | −10.742 | 1.00 | 0.00 | H |
| ATOM | 3963 | 3HG1 | VAL | A | 419 | 1.711 | 73.042 | −10.516 | 1.00 | 0.00 | H |
| ATOM | 3964 | 1HG2 | VAL | A | 419 | 4.094 | 74.849 | −9.886 | 1.00 | 0.00 | H |
| ATOM | 3965 | 2HG2 | VAL | A | 419 | 4.178 | 73.432 | −10.960 | 1.00 | 0.00 | H |
| ATOM | 3966 | 3HG2 | VAL | A | 419 | 4.717 | 75.025 | −11.543 | 1.00 | 0.00 | H |
| ATOM | 3967 | N | VAL | A | 420 | −0.036 | 74.140 | −13.519 | 1.00 | 0.00 | N |
| ATOM | 3968 | CA | VAL | A | 420 | −1.347 | 74.545 | −14.013 | 1.00 | 0.00 | C |
| ATOM | 3969 | C | VAL | A | 420 | −2.414 | 73.741 | −13.265 | 1.00 | 0.00 | C |
| ATOM | 3970 | O | VAL | A | 420 | −2.167 | 72.596 | −12.896 | 1.00 | 0.00 | O |
| ATOM | 3971 | CB | VAL | A | 420 | −1.454 | 74.318 | −15.536 | 1.00 | 0.00 | C |
| ATOM | 3972 | CG1 | VAL | A | 420 | −0.665 | 75.370 | −16.326 | 1.00 | 0.00 | C |
| ATOM | 3973 | CG2 | VAL | A | 420 | −0.990 | 72.920 | −15.970 | 1.00 | 0.00 | C |
| ATOM | 3974 | H | VAL | A | 420 | 0.035 | 73.172 | −13.237 | 1.00 | 0.00 | H |
| ATOM | 3975 | HA | VAL | A | 420 | −1.476 | 75.617 | −13.866 | 1.00 | 0.00 | H |
| ATOM | 3976 | HB | VAL | A | 420 | −2.503 | 74.317 | −15.831 | 1.00 | 0.00 | H |
| ATOM | 3977 | 1HG1 | VAL | A | 420 | −0.766 | 75.174 | −17.394 | 1.00 | 0.00 | H |
| ATOM | 3978 | 2HG1 | VAL | A | 420 | −1.055 | 76.362 | −16.100 | 1.00 | 0.00 | H |
| ATOM | 3979 | 3HG1 | VAL | A | 420 | 0.387 | 75.321 | −16.046 | 1.00 | 0.00 | H |
| ATOM | 3980 | 1HG2 | VAL | A | 420 | −1.090 | 72.823 | −17.051 | 1.00 | 0.00 | H |
| ATOM | 3981 | 2HG2 | VAL | A | 420 | 0.053 | 72.778 | −15.688 | 1.00 | 0.00 | H |
| ATOM | 3982 | 3HG2 | VAL | A | 420 | −1.604 | 72.164 | −15.480 | 1.00 | 0.00 | H |
| ATOM | 3983 | N | TRP | A | 421 | −3.594 | 74.320 | −13.036 | 1.00 | 0.00 | N |
| ATOM | 3984 | CA | TRP | A | 421 | −4.707 | 73.587 | −12.446 | 1.00 | 0.00 | C |
| ATOM | 3985 | C | TRP | A | 421 | −5.363 | 72.772 | −13.560 | 1.00 | 0.00 | C |
| ATOM | 3986 | O | TRP | A | 421 | −5.759 | 73.346 | −14.573 | 1.00 | 0.00 | O |
| ATOM | 3987 | CB | TRP | A | 421 | −5.701 | 74.560 | −11.796 | 1.00 | 0.00 | C |
| ATOM | 3988 | CG | TRP | A | 421 | −6.680 | 73.970 | −10.819 | 1.00 | 0.00 | C |
| ATOM | 3989 | CD1 | TRP | A | 421 | −7.447 | 72.870 | −11.010 | 1.00 | 0.00 | C |
| ATOM | 3990 | CD2 | TRP | A | 421 | −7.021 | 74.451 | −9.482 | 1.00 | 0.00 | C |
| ATOM | 3991 | CE2 | TRP | A | 421 | −7.995 | 73.572 | −8.919 | 1.00 | 0.00 | C |
| ATOM | 3992 | CE3 | TRP | A | 421 | −6.623 | 75.544 | −8.679 | 1.00 | 0.00 | C |
| ATOM | 3993 | NE1 | TRP | A | 421 | −8.207 | 72.619 | −9.889 | 1.00 | 0.00 | N |
| ATOM | 3994 | CZ2 | TRP | A | 421 | −8.530 | 73.765 | −7.636 | 1.00 | 0.00 | C |
| ATOM | 3995 | CZ3 | TRP | A | 421 | −7.159 | 75.751 | −7.395 | 1.00 | 0.00 | C |
| ATOM | 3996 | CH2 | TRP | A | 421 | −8.114 | 74.864 | −6.872 | 1.00 | 0.00 | C |
| ATOM | 3997 | H | TRP | A | 421 | −3.735 | 75.291 | −13.274 | 1.00 | 0.00 | H |
| ATOM | 3998 | HA | TRP | A | 421 | −4.338 | 72.954 | −11.639 | 1.00 | 0.00 | H |
| ATOM | 3999 | 1HB | TRP | A | 421 | −5.153 | 75.324 | −11.243 | 1.00 | 0.00 | H |
| ATOM | 4000 | 2HB | TRP | A | 421 | −6.303 | 75.036 | −12.570 | 1.00 | 0.00 | H |
| ATOM | 4001 | HD1 | TRP | A | 421 | −7.397 | 72.333 | −11.945 | 1.00 | 0.00 | H |
| ATOM | 4002 | HE3 | TRP | A | 421 | −5.890 | 76.246 | −9.048 | 1.00 | 0.00 | H |
| ATOM | 4003 | HE1 | TRP | A | 421 | −8.819 | 71.816 | −9.863 | 1.00 | 0.00 | H |
| ATOM | 4004 | HZ2 | TRP | A | 421 | −9.257 | 73.070 | −7.244 | 1.00 | 0.00 | H |
| ATOM | 4005 | HZ3 | TRP | A | 421 | −6.837 | 76.596 | −6.805 | 1.00 | 0.00 | H |
| ATOM | 4006 | HH2 | TRP | A | 421 | −8.527 | 75.024 | −5.887 | 1.00 | 0.00 | H |
| ATOM | 4007 | N | VAL | A | 422 | −5.481 | 71.456 | −13.386 | 1.00 | 0.00 | N |
| ATOM | 4008 | CA | VAL | A | 422 | −6.138 | 70.560 | −14.328 | 1.00 | 0.00 | C |
| ATOM | 4009 | C | VAL | A | 422 | −6.677 | 69.397 | −13.496 | 1.00 | 0.00 | C |
| ATOM | 4010 | O | VAL | A | 422 | −5.898 | 68.751 | −12.796 | 1.00 | 0.00 | O |
| ATOM | 4011 | CB | VAL | A | 422 | −5.141 | 70.046 | −15.390 | 1.00 | 0.00 | C |
| ATOM | 4012 | CG1 | VAL | A | 422 | −5.822 | 69.063 | −16.351 | 1.00 | 0.00 | C |
| ATOM | 4013 | CG2 | VAL | A | 422 | −4.514 | 71.165 | −16.233 | 1.00 | 0.00 | C |
| ATOM | 4014 | H | VAL | A | 422 | −5.101 | 71.020 | −12.558 | 1.00 | 0.00 | H |
| ATOM | 4015 | HA | VAL | A | 422 | −6.947 | 71.091 | −14.831 | 1.00 | 0.00 | H |
| ATOM | 4016 | HB | VAL | A | 422 | −4.301 | 69.559 | −14.895 | 1.00 | 0.00 | H |
| ATOM | 4017 | 1HG1 | VAL | A | 422 | −5.099 | 68.715 | −17.089 | 1.00 | 0.00 | H |
| ATOM | 4018 | 2HG1 | VAL | A | 422 | −6.205 | 68.211 | −15.789 | 1.00 | 0.00 | H |
| ATOM | 4019 | 3HG1 | VAL | A | 422 | −6.647 | 69.563 | −16.858 | 1.00 | 0.00 | H |
| ATOM | 4020 | 1HG2 | VAL | A | 422 | −3.825 | 70.733 | −16.958 | 1.00 | 0.00 | H |
| ATOM | 4021 | 2HG2 | VAL | A | 422 | −5.299 | 71.709 | −16.758 | 1.00 | 0.00 | H |
| ATOM | 4022 | 3HG2 | VAL | A | 422 | −3.972 | 71.851 | −15.581 | 1.00 | 0.00 | H |
| ATOM | 4023 | N | GLU | A | 423 | −7.981 | 69.130 | −13.572 | 1.00 | 0.00 | N |
| ATOM | 4024 | CA | GLU | A | 423 | −8.638 | 68.137 | −12.736 | 1.00 | 0.00 | C |
| ATOM | 4025 | C | GLU | A | 423 | −8.498 | 66.732 | −13.317 | 1.00 | 0.00 | C |
| ATOM | 4026 | O | GLU | A | 423 | −8.254 | 66.566 | −14.510 | 1.00 | 0.00 | O |
| ATOM | 4027 | CB | GLU | A | 423 | −10.124 | 68.487 | −12.583 | 1.00 | 0.00 | C |
| ATOM | 4028 | CG | GLU | A | 423 | −10.326 | 69.693 | −11.658 | 1.00 | 0.00 | C |
| ATOM | 4029 | CD | GLU | A | 423 | −9.865 | 69.403 | −10.238 | 1.00 | 0.00 | C |
| ATOM | 4030 | OE1 | GLU | A | 423 | −9.641 | 68.212 | −9.923 | 1.00 | 0.00 | O |
| ATOM | 4031 | OE2 | GLU | A | 423 | −9.687 | 70.391 | −9.499 | 1.00 | 0.00 | O1− |
| ATOM | 4032 | H | GLU | A | 423 | −8.561 | 69.627 | −14.234 | 1.00 | 0.00 | H |
| ATOM | 4033 | HA | GLU | A | 423 | −8.212 | 68.167 | −11.733 | 1.00 | 0.00 | H |
| ATOM | 4034 | 1HB | GLU | A | 423 | −10.544 | 68.727 | −13.559 | 1.00 | 0.00 | H |
| ATOM | 4035 | 2HB | GLU | A | 423 | −10.656 | 67.635 | −12.159 | 1.00 | 0.00 | H |
| ATOM | 4036 | 1HG | GLU | A | 423 | −9.754 | 70.541 | −12.036 | 1.00 | 0.00 | H |
| ATOM | 4037 | 2HG | GLU | A | 423 | −11.384 | 69.954 | −11.626 | 1.00 | 0.00 | H |
| ATOM | 4038 | N | GLU | A | 424 | −8.654 | 65.731 | −12.447 | 1.00 | 0.00 | N |
| ATOM | 4039 | CA | GLU | A | 424 | −8.469 | 64.305 | −12.682 | 1.00 | 0.00 | C |
| ATOM | 4040 | C | GLU | A | 424 | −8.888 | 63.825 | −14.079 | 1.00 | 0.00 | C |
| ATOM | 4041 | O | GLU | A | 424 | −8.233 | 62.953 | −14.649 | 1.00 | 0.00 | O |
| ATOM | 4042 | CB | GLU | A | 424 | −9.251 | 63.507 | −11.629 | 1.00 | 0.00 | C |
| ATOM | 4043 | CG | GLU | A | 424 | −8.908 | 63.856 | −10.175 | 1.00 | 0.00 | C |
| ATOM | 4044 | CD | GLU | A | 424 | −7.475 | 63.517 | −9.831 | 1.00 | 0.00 | C |
| ATOM | 4045 | OE1 | GLU | A | 424 | −7.230 | 62.366 | −9.421 | 1.00 | 0.00 | O |
| ATOM | 4046 | OE2 | GLU | A | 424 | −6.620 | 64.423 | −9.823 | 1.00 | 0.00 | O1− |
| ATOM | 4047 | H | GLU | A | 424 | −8.934 | 65.927 | −11.497 | 1.00 | 0.00 | H |
| ATOM | 4048 | HA | GLU | A | 424 | −7.423 | 64.042 | −12.520 | 1.00 | 0.00 | H |
| ATOM | 4049 | 1HB | GLU | A | 424 | −10.319 | 63.689 | −11.754 | 1.00 | 0.00 | H |
| ATOM | 4050 | 2HB | GLU | A | 424 | −9.048 | 62.443 | −11.754 | 1.00 | 0.00 | H |
| ATOM | 4051 | 1HG | GLU | A | 424 | −9.051 | 64.925 | −10.014 | 1.00 | 0.00 | H |
| ATOM | 4052 | 2HG | GLU | A | 424 | −9.559 | 63.298 | −9.503 | 1.00 | 0.00 | H |
| ATOM | 4053 | N | SER | A | 425 | −10.017 | 64.315 | −14.591 | 1.00 | 0.00 | N |
| ATOM | 4054 | CA | SER | A | 425 | −10.503 | 63.979 | −15.914 | 1.00 | 0.00 | C |
| ATOM | 4055 | C | SER | A | 425 | −9.444 | 64.333 | −16.955 | 1.00 | 0.00 | C |
| ATOM | 4056 | O | SER | A | 425 | −9.004 | 63.502 | −17.751 | 1.00 | 0.00 | O |
| ATOM | 4057 | CB | SER | A | 425 | −11.790 | 64.777 | −16.168 | 1.00 | 0.00 | C |
| ATOM | 4058 | OG | SER | A | 425 | −11.685 | 66.090 | −15.636 | 1.00 | 0.00 | O |
| ATOM | 4059 | H | SER | A | 425 | −10.581 | 64.955 | −14.049 | 1.00 | 0.00 | H |
| ATOM | 4060 | HA | SER | A | 425 | −10.722 | 62.912 | −15.961 | 1.00 | 0.00 | H |
| ATOM | 4061 | 1HB | SER | A | 425 | −11.969 | 64.848 | −17.241 | 1.00 | 0.00 | H |
| ATOM | 4062 | 2HB | SER | A | 425 | −12.631 | 64.273 | −15.692 | 1.00 | 0.00 | H |
| ATOM | 4063 | HG | SER | A | 425 | −12.499 | 66.570 | −15.805 | 1.00 | 0.00 | H |
| ATOM | 4064 | N | ASP | A | 426 | −9.071 | 65.603 | −16.951 | 1.00 | 0.00 | N |
| ATOM | 4065 | CA | ASP | A | 426 | −8.343 | 66.239 | −18.024 | 1.00 | 0.00 | C |
| ATOM | 4066 | C | ASP | A | 426 | −6.851 | 66.160 | −17.747 | 1.00 | 0.00 | C |
| ATOM | 4067 | O | ASP | A | 426 | −6.035 | 66.545 | −18.583 | 1.00 | 0.00 | O |
| ATOM | 4068 | CB | ASP | A | 426 | −8.869 | 67.655 | −18.235 | 1.00 | 0.00 | C |
| ATOM | 4069 | CG | ASP | A | 426 | −10.282 | 67.553 | −18.775 | 1.00 | 0.00 | C |
| ATOM | 4070 | OD1 | ASP | A | 426 | −10.398 | 67.429 | −20.014 | 1.00 | 0.00 | O |
| ATOM | 4071 | OD2 | ASP | A | 426 | −11.209 | 67.502 | −17.938 | 1.00 | 0.00 | O1− |
| ATOM | 4072 | H | ASP | A | 426 | −9.293 | 66.193 | −16.162 | 1.00 | 0.00 | H |
| ATOM | 4073 | HA | ASP | A | 426 | −8.648 | 65.806 | −18.977 | 1.00 | 0.00 | H |
| ATOM | 4074 | 1HB | ASP | A | 426 | −8.866 | 68.189 | −17.285 | 1.00 | 0.00 | H |
| ATOM | 4075 | 2HB | ASP | A | 426 | −8.230 | 68.178 | −18.947 | 1.00 | 0.00 | H |
| ATOM | 4076 | N | LYS | A | 427 | −6.485 | 65.551 | −16.618 | 1.00 | 0.00 | N |
| ATOM | 4077 | CA | LYS | A | 427 | −5.171 | 64.966 | −16.454 | 1.00 | 0.00 | C |
| ATOM | 4078 | C | LYS | A | 427 | −5.010 | 63.752 | −17.389 | 1.00 | 0.00 | C |
| ATOM | 4079 | O | LYS | A | 427 | −3.941 | 63.147 | −17.413 | 1.00 | 0.00 | O |
| ATOM | 4080 | CB | LYS | A | 427 | −4.909 | 64.577 | −14.991 | 1.00 | 0.00 | C |
| ATOM | 4081 | CG | LYS | A | 427 | −4.964 | 65.747 | −13.995 | 1.00 | 0.00 | C |
| ATOM | 4082 | CD | LYS | A | 427 | −4.552 | 65.272 | −12.591 | 1.00 | 0.00 | C |
| ATOM | 4083 | CE | LYS | A | 427 | −4.792 | 66.348 | −11.521 | 1.00 | 0.00 | C |
| ATOM | 4084 | NZ | LYS | A | 427 | −4.477 | 65.863 | −10.168 | 1.00 | 0.00 | N1+ |
| ATOM | 4085 | H | LYS | A | 427 | −7.133 | 65.488 | −15.846 | 1.00 | 0.00 | H |
| ATOM | 4086 | HA | LYS | A | 427 | −4.410 | 65.726 | −16.632 | 1.00 | 0.00 | H |
| ATOM | 4087 | 1HB | LYS | A | 427 | −5.657 | 63.854 | −14.668 | 1.00 | 0.00 | H |
| ATOM | 4088 | 2HB | LYS | A | 427 | −3.916 | 64.136 | −14.904 | 1.00 | 0.00 | H |
| ATOM | 4089 | 1HG | LYS | A | 427 | −4.282 | 66.533 | −14.320 | 1.00 | 0.00 | H |
| ATOM | 4090 | 2HG | LYS | A | 427 | −5.979 | 66.142 | −13.954 | 1.00 | 0.00 | H |
| ATOM | 4091 | 1HD | LYS | A | 427 | −5.132 | 64.390 | −12.320 | 1.00 | 0.00 | H |
| ATOM | 4092 | 2HD | LYS | A | 427 | −3.491 | 65.023 | −12.588 | 1.00 | 0.00 | H |
| ATOM | 4093 | 1HE | LYS | A | 427 | −4.161 | 67.213 | −11.727 | 1.00 | 0.00 | H |
| ATOM | 4094 | 2HE | LYS | A | 427 | −5.839 | 66.651 | −11.538 | 1.00 | 0.00 | H |
| ATOM | 4095 | 1HZ | LYS | A | 427 | −4.648 | 66.599 | −9.497 | 1.00 | 0.00 | H |
| ATOM | 4096 | 2HZ | LYS | A | 427 | −5.061 | 65.068 | −9.951 | 1.00 | 0.00 | H |
| ATOM | 4097 | 3HZ | LYS | A | 427 | −3.506 | 65.589 | −10.126 | 1.00 | 0.00 | H |
| ATOM | 4098 | N | ARG | A | 428 | −6.045 | 63.378 | −18.156 | 1.00 | 0.00 | N |
| ATOM | 4099 | CA | ARG | A | 428 | −5.901 | 62.563 | −19.353 | 1.00 | 0.00 | C |
| ATOM | 4100 | C | ARG | A | 428 | −5.848 | 63.480 | −20.580 | 1.00 | 0.00 | C |
| ATOM | 4101 | O | ARG | A | 428 | −4.893 | 63.427 | −21.349 | 1.00 | 0.00 | O |
| ATOM | 4102 | CB | ARG | A | 428 | −7.035 | 61.537 | −19.494 | 1.00 | 0.00 | C |
| ATOM | 4103 | CG | ARG | A | 428 | −7.023 | 60.491 | −18.371 | 1.00 | 0.00 | C |
| ATOM | 4104 | CD | ARG | A | 428 | −7.880 | 59.279 | −18.768 | 1.00 | 0.00 | C |
| ATOM | 4105 | NE | ARG | A | 428 | −7.865 | 58.239 | −17.723 | 1.00 | 0.00 | N |
| ATOM | 4106 | CZ | ARG | A | 428 | −8.117 | 56.932 | −17.904 | 1.00 | 0.00 | C |
| ATOM | 4107 | NH1 | ARG | A | 428 | −8.430 | 56.443 | −19.103 | 1.00 | 0.00 | N1+ |
| ATOM | 4108 | NH2 | ARG | A | 428 | −8.033 | 56.092 | −16.883 | 1.00 | 0.00 | N |
| ATOM | 4109 | H | ARG | A | 428 | −6.983 | 63.663 | −17.913 | 1.00 | 0.00 | H |
| ATOM | 4110 | HA | ARG | A | 428 | −5.008 | 61.944 | −19.267 | 1.00 | 0.00 | H |
| ATOM | 4111 | 1HB | ARG | A | 428 | −7.996 | 62.050 | −19.464 | 1.00 | 0.00 | H |
| ATOM | 4112 | 2HB | ARG | A | 428 | −6.934 | 61.011 | −20.443 | 1.00 | 0.00 | H |
| ATOM | 4113 | 1HG | ARG | A | 428 | −5.999 | 60.162 | −18.191 | 1.00 | 0.00 | H |
| ATOM | 4114 | 2HG | ARG | A | 428 | −7.427 | 60.932 | −17.459 | 1.00 | 0.00 | H |
| ATOM | 4115 | 1HD | ARG | A | 428 | −8.911 | 59.597 | −18.923 | 1.00 | 0.00 | H |
| ATOM | 4116 | 2HD | ARG | A | 428 | −7.492 | 58.845 | −19.690 | 1.00 | 0.00 | H |
| ATOM | 4117 | HE | ARG | A | 428 | −7.641 | 58.587 | −16.802 | 1.00 | 0.00 | H |
| ATOM | 4118 | 1HH1 | ARG | A | 428 | −8.615 | 55.456 | −19.213 | 1.00 | 0.00 | H |
| ATOM | 4119 | 2HH1 | ARG | A | 428 | −8.484 | 57.058 | −19.902 | 1.00 | 0.00 | H |
| ATOM | 4120 | 1HH2 | ARG | A | 428 | −8.222 | 55.109 | −17.021 | 1.00 | 0.00 | H |
| ATOM | 4121 | 2HH2 | ARG | A | 428 | −7.780 | 56.434 | −15.967 | 1.00 | 0.00 | H |
| ATOM | 4122 | N | SER | A | 429 | −6.879 | 64.297 | −20.794 | 1.00 | 0.00 | N |
| ATOM | 4123 | CA | SER | A | 429 | −7.008 | 65.135 | −21.976 | 1.00 | 0.00 | C |
| ATOM | 4124 | C | SER | A | 429 | −5.747 | 65.967 | −22.254 | 1.00 | 0.00 | C |
| ATOM | 4125 | O | SER | A | 429 | −5.261 | 66.020 | −23.382 | 1.00 | 0.00 | O |
| ATOM | 4126 | CB | SER | A | 429 | −8.229 | 66.041 | −21.799 | 1.00 | 0.00 | C |
| ATOM | 4127 | OG | SER | A | 429 | −9.324 | 65.301 | −21.285 | 1.00 | 0.00 | O |
| ATOM | 4128 | H | SER | A | 429 | −7.625 | 64.357 | −20.116 | 1.00 | 0.00 | H |
| ATOM | 4129 | HA | SER | A | 429 | −7.247 | 64.514 | −22.839 | 1.00 | 0.00 | H |
| ATOM | 4130 | 1HB | SER | A | 429 | −7.988 | 66.846 | −21.104 | 1.00 | 0.00 | H |
| ATOM | 4131 | 2HB | SER | A | 429 | −8.510 | 66.467 | −22.762 | 1.00 | 0.00 | H |
| ATOM | 4132 | HG | SER | A | 429 | −10.081 | 65.882 | −21.180 | 1.00 | 0.00 | H |
| ATOM | 4133 | N | PHE | A | 430 | −5.236 | 66.650 | −21.230 | 1.00 | 0.00 | N |
| ATOM | 4134 | CA | PHE | A | 430 | −4.116 | 67.571 | −21.357 | 1.00 | 0.00 | C |
| ATOM | 4135 | C | PHE | A | 430 | −2.817 | 66.766 | −21.492 | 1.00 | 0.00 | C |
| ATOM | 4136 | O | PHE | A | 430 | −1.821 | 67.237 | −22.034 | 1.00 | 0.00 | O |
| ATOM | 4137 | CB | PHE | A | 430 | −4.095 | 68.475 | −20.111 | 1.00 | 0.00 | C |
| ATOM | 4138 | CG | PHE | A | 430 | −3.428 | 69.834 | −20.252 | 1.00 | 0.00 | C |
| ATOM | 4139 | CD1 | PHE | A | 430 | −4.198 | 71.010 | −20.143 | 1.00 | 0.00 | C |
| ATOM | 4140 | CD2 | PHE | A | 430 | −2.040 | 69.957 | −20.466 | 1.00 | 0.00 | C |
| ATOM | 4141 | CE1 | PHE | A | 430 | −3.597 | 72.277 | −20.240 | 1.00 | 0.00 | C |
| ATOM | 4142 | CE2 | PHE | A | 430 | −1.437 | 71.222 | −20.573 | 1.00 | 0.00 | C |
| ATOM | 4143 | CZ | PHE | A | 430 | −2.215 | 72.385 | −20.458 | 1.00 | 0.00 | C |
| ATOM | 4144 | H | PHE | A | 430 | −5.627 | 66.541 | −20.305 | 1.00 | 0.00 | H |
| ATOM | 4145 | HA | PHE | A | 430 | −4.269 | 68.212 | −22.225 | 1.00 | 0.00 | H |
| ATOM | 4146 | 1HB | PHE | A | 430 | −5.118 | 68.684 | −19.796 | 1.00 | 0.00 | H |
| ATOM | 4147 | 2HB | PHE | A | 430 | −3.562 | 67.972 | −19.305 | 1.00 | 0.00 | H |
| ATOM | 4148 | HD1 | PHE | A | 430 | −5.264 | 70.945 | −19.982 | 1.00 | 0.00 | H |
| ATOM | 4149 | HD2 | PHE | A | 430 | −1.426 | 69.073 | −20.550 | 1.00 | 0.00 | H |
| ATOM | 4150 | HE1 | PHE | A | 430 | −4.200 | 73.169 | −20.148 | 1.00 | 0.00 | H |
| ATOM | 4151 | HE2 | PHE | A | 430 | −0.373 | 71.300 | −20.744 | 1.00 | 0.00 | H |
| ATOM | 4152 | HZ | PHE | A | 430 | −1.754 | 73.359 | −20.536 | 1.00 | 0.00 | H |
| ATOM | 4153 | N | LEU | A | 431 | −2.812 | 65.547 | −20.954 | 1.00 | 0.00 | N |
| ATOM | 4154 | CA | LEU | A | 431 | −1.630 | 64.705 | −20.869 | 1.00 | 0.00 | C |
| ATOM | 4155 | C | LEU | A | 431 | −1.194 | 64.244 | −22.265 | 1.00 | 0.00 | C |
| ATOM | 4156 | O | LEU | A | 431 | −0.060 | 63.801 | −22.444 | 1.00 | 0.00 | O |
| ATOM | 4157 | CB | LEU | A | 431 | −1.951 | 63.546 | −19.909 | 1.00 | 0.00 | C |
| ATOM | 4158 | CG | LEU | A | 431 | −0.852 | 62.510 | −19.653 | 1.00 | 0.00 | C |
| ATOM | 4159 | CD1 | LEU | A | 431 | 0.341 | 63.123 | −18.923 | 1.00 | 0.00 | C |
| ATOM | 4160 | CD2 | LEU | A | 431 | −1.409 | 61.370 | −18.792 | 1.00 | 0.00 | C |
| ATOM | 4161 | H | LEU | A | 431 | −3.664 | 65.159 | −20.576 | 1.00 | 0.00 | H |
| ATOM | 4162 | HA | LEU | A | 431 | −0.842 | 65.238 | −20.338 | 1.00 | 0.00 | H |
| ATOM | 4163 | 1HB | LEU | A | 431 | −2.201 | 63.947 | −18.927 | 1.00 | 0.00 | H |
| ATOM | 4164 | 2HB | LEU | A | 431 | −2.797 | 62.977 | −20.295 | 1.00 | 0.00 | H |
| ATOM | 4165 | HG | LEU | A | 431 | −0.502 | 62.107 | −20.604 | 1.00 | 0.00 | H |
| ATOM | 4166 | 1HD1 | LEU | A | 431 | 1.100 | 62.358 | −18.759 | 1.00 | 0.00 | H |
| ATOM | 4167 | 2HD1 | LEU | A | 431 | 0.762 | 63.928 | −19.526 | 1.00 | 0.00 | H |
| ATOM | 4168 | 3HD1 | LEU | A | 431 | 0.014 | 63.522 | −17.963 | 1.00 | 0.00 | H |
| ATOM | 4169 | 1HD2 | LEU | A | 431 | −0.626 | 60.634 | −18.611 | 1.00 | 0.00 | H |
| ATOM | 4170 | 2HD2 | LEU | A | 431 | −1.757 | 61.771 | −17.840 | 1.00 | 0.00 | H |
| ATOM | 4171 | 3HD2 | LEU | A | 431 | −2.241 | 60.895 | −19.311 | 1.00 | 0.00 | H |
| ATOM | 4172 | N | LEU | A | 432 | −2.092 | 64.336 | −23.248 | 1.00 | 0.00 | N |
| ATOM | 4173 | CA | LEU | A | 432 | −1.822 | 63.954 | −24.622 | 1.00 | 0.00 | C |
| ATOM | 4174 | C | LEU | A | 432 | −0.555 | 64.632 | −25.149 | 1.00 | 0.00 | C |
| ATOM | 4175 | O | LEU | A | 432 | 0.349 | 63.940 | −25.620 | 1.00 | 0.00 | O |
| ATOM | 4176 | CB | LEU | A | 432 | −3.027 | 64.266 | −25.521 | 1.00 | 0.00 | C |
| ATOM | 4177 | CG | LEU | A | 432 | −4.257 | 63.385 | −25.238 | 1.00 | 0.00 | C |
| ATOM | 4178 | CD1 | LEU | A | 432 | −5.444 | 63.902 | −26.060 | 1.00 | 0.00 | C |
| ATOM | 4179 | CD2 | LEU | A | 432 | −4.009 | 61.916 | −25.610 | 1.00 | 0.00 | C |
| ATOM | 4180 | H | LEU | A | 432 | −3.018 | 64.689 | −23.054 | 1.00 | 0.00 | H |
| ATOM | 4181 | HA | LEU | A | 432 | −1.757 | 62.868 | −24.691 | 1.00 | 0.00 | H |
| ATOM | 4182 | 1HB | LEU | A | 432 | −3.331 | 65.302 | −25.374 | 1.00 | 0.00 | H |
| ATOM | 4183 | 2HB | LEU | A | 432 | −2.753 | 64.113 | −26.565 | 1.00 | 0.00 | H |
| ATOM | 4184 | HG | LEU | A | 432 | −4.499 | 63.427 | −24.176 | 1.00 | 0.00 | H |
| ATOM | 4185 | 1HD1 | LEU | A | 432 | −6.319 | 63.283 | −25.865 | 1.00 | 0.00 | H |
| ATOM | 4186 | 2HD1 | LEU | A | 432 | −5.659 | 64.933 | −25.780 | 1.00 | 0.00 | H |
| ATOM | 4187 | 3HD1 | LEU | A | 432 | −5.198 | 63.859 | −27.121 | 1.00 | 0.00 | H |
| ATOM | 4188 | 1HD2 | LEU | A | 432 | −4.902 | 61.330 | −25.395 | 1.00 | 0.00 | H |
| ATOM | 4189 | 2HD2 | LEU | A | 432 | −3.776 | 61.844 | −26.672 | 1.00 | 0.00 | H |
| ATOM | 4190 | 3HD2 | LEU | A | 432 | −3.172 | 61.530 | −25.028 | 1.00 | 0.00 | H |
| ATOM | 4191 | N | ASP | A | 433 | −0.487 | 65.966 | −25.115 | 1.00 | 0.00 | N |
| ATOM | 4192 | CA | ASP | A | 433 | 0.691 | 66.680 | −25.604 | 1.00 | 0.00 | C |
| ATOM | 4193 | C | ASP | A | 433 | 1.906 | 66.236 | −24.800 | 1.00 | 0.00 | C |
| ATOM | 4194 | O | ASP | A | 433 | 2.933 | 65.840 | −25.350 | 1.00 | 0.00 | O |
| ATOM | 4195 | CB | ASP | A | 433 | 0.541 | 68.198 | −25.482 | 1.00 | 0.00 | C |
| ATOM | 4196 | CG | ASP | A | 433 | 1.872 | 68.860 | −25.824 | 1.00 | 0.00 | C |
| ATOM | 4197 | OD1 | ASP | A | 433 | 2.156 | 68.973 | −27.035 | 1.00 | 0.00 | O |
| ATOM | 4198 | OD2 | ASP | A | 433 | 2.604 | 69.205 | −24.872 | 1.00 | 0.00 | O1− |
| ATOM | 4199 | H | ASP | A | 433 | −1.259 | 66.502 | −24.746 | 1.00 | 0.00 | H |
| ATOM | 4200 | HA | ASP | A | 433 | 0.836 | 66.462 | −26.662 | 1.00 | 0.00 | H |
| ATOM | 4201 | 1HB | ASP | A | 433 | −0.228 | 68.544 | −26.172 | 1.00 | 0.00 | H |
| ATOM | 4202 | 2HB | ASP | A | 433 | 0.255 | 68.454 | −24.462 | 1.00 | 0.00 | H |
| ATOM | 4203 | N | LEU | A | 434 | 1.760 | 66.290 | −23.479 | 1.00 | 0.00 | N |
| ATOM | 4204 | CA | LEU | A | 434 | 2.867 | 66.138 | −22.559 | 1.00 | 0.00 | C |
| ATOM | 4205 | C | LEU | A | 434 | 3.579 | 64.794 | −22.760 | 1.00 | 0.00 | C |
| ATOM | 4206 | O | LEU | A | 434 | 4.769 | 64.672 | −22.452 | 1.00 | 0.00 | O |
| ATOM | 4207 | CB | LEU | A | 434 | 2.371 | 66.285 | −21.113 | 1.00 | 0.00 | C |
| ATOM | 4208 | CG | LEU | A | 434 | 1.598 | 67.580 | −20.802 | 1.00 | 0.00 | C |
| ATOM | 4209 | CD1 | LEU | A | 434 | 1.275 | 67.611 | −19.305 | 1.00 | 0.00 | C |
| ATOM | 4210 | CD2 | LEU | A | 434 | 2.358 | 68.863 | −21.159 | 1.00 | 0.00 | C |
| ATOM | 4211 | H | LEU | A | 434 | 0.845 | 66.442 | −23.078 | 1.00 | 0.00 | H |
| ATOM | 4212 | HA | LEU | A | 434 | 3.558 | 66.972 | −22.680 | 1.00 | 0.00 | H |
| ATOM | 4213 | 1HB | LEU | A | 434 | 1.698 | 65.462 | −20.874 | 1.00 | 0.00 | H |
| ATOM | 4214 | 2HB | LEU | A | 434 | 3.223 | 66.266 | −20.433 | 1.00 | 0.00 | H |
| ATOM | 4215 | HG | LEU | A | 434 | 0.683 | 67.609 | −21.394 | 1.00 | 0.00 | H |
| ATOM | 4216 | 1HD1 | LEU | A | 434 | 0.728 | 68.524 | −19.069 | 1.00 | 0.00 | H |
| ATOM | 4217 | 2HD1 | LEU | A | 434 | 0.666 | 66.745 | −19.046 | 1.00 | 0.00 | H |
| ATOM | 4218 | 3HD1 | LEU | A | 434 | 2.203 | 67.587 | −18.732 | 1.00 | 0.00 | H |
| ATOM | 4219 | 1HD2 | LEU | A | 434 | 1.746 | 69.729 | −20.909 | 1.00 | 0.00 | H |
| ATOM | 4220 | 2HD2 | LEU | A | 434 | 3.290 | 68.902 | −20.596 | 1.00 | 0.00 | H |
| ATOM | 4221 | 3HD2 | LEU | A | 434 | 2.579 | 68.869 | −22.226 | 1.00 | 0.00 | H |
| ATOM | 4222 | N | LEU | A | 435 | 2.849 | 63.788 | −23.254 | 1.00 | 0.00 | N |
| ATOM | 4223 | CA | LEU | A | 435 | 3.371 | 62.470 | −23.567 | 1.00 | 0.00 | C |
| ATOM | 4224 | C | LEU | A | 435 | 3.720 | 62.343 | −25.047 | 1.00 | 0.00 | C |
| ATOM | 4225 | O | LEU | A | 435 | 4.697 | 61.673 | −25.378 | 1.00 | 0.00 | O |
| ATOM | 4226 | CB | LEU | A | 435 | 2.366 | 61.384 | −23.187 | 1.00 | 0.00 | C |
| ATOM | 4227 | CG | LEU | A | 435 | 2.093 | 61.305 | −21.681 | 1.00 | 0.00 | C |
| ATOM | 4228 | CD1 | LEU | A | 435 | 1.127 | 60.148 | −21.450 | 1.00 | 0.00 | C |
| ATOM | 4229 | CD2 | LEU | A | 435 | 3.354 | 61.070 | −20.848 | 1.00 | 0.00 | C |
| ATOM | 4230 | H | LEU | A | 435 | 1.864 | 63.919 | −23.433 | 1.00 | 0.00 | H |
| ATOM | 4231 | HA | LEU | A | 435 | 4.252 | 62.273 | −22.956 | 1.00 | 0.00 | H |
| ATOM | 4232 | 1HB | LEU | A | 435 | 1.415 | 61.581 | −23.682 | 1.00 | 0.00 | H |
| ATOM | 4233 | 2HB | LEU | A | 435 | 2.745 | 60.412 | −23.502 | 1.00 | 0.00 | H |
| ATOM | 4234 | HG | LEU | A | 435 | 1.656 | 62.245 | −21.340 | 1.00 | 0.00 | H |
| ATOM | 4235 | 1HD1 | LEU | A | 435 | 0.909 | 60.062 | −20.386 | 1.00 | 0.00 | H |
| ATOM | 4236 | 2HD1 | LEU | A | 435 | 0.201 | 60.331 | −21.996 | 1.00 | 0.00 | H |
| ATOM | 4237 | 3HD1 | LEU | A | 435 | 1.579 | 59.221 | −21.804 | 1.00 | 0.00 | H |
| ATOM | 4238 | 1HD2 | LEU | A | 435 | 3.090 | 61.025 | −19.791 | 1.00 | 0.00 | H |
| ATOM | 4239 | 2HD2 | LEU | A | 435 | 3.818 | 60.130 | −21.147 | 1.00 | 0.00 | H |
| ATOM | 4240 | 3HD2 | LEU | A | 435 | 4.056 | 61.889 | −21.011 | 1.00 | 0.00 | H |
| ATOM | 4241 | N | ASN | A | 436 | 2.972 | 62.970 | −25.954 | 1.00 | 0.00 | N |
| ATOM | 4242 | CA | ASN | A | 436 | 3.369 | 63.012 | −27.362 | 1.00 | 0.00 | C |
| ATOM | 4243 | C | ASN | A | 436 | 4.712 | 63.733 | −27.484 | 1.00 | 0.00 | C |
| ATOM | 4244 | O | ASN | A | 436 | 5.469 | 63.523 | −28.430 | 1.00 | 0.00 | O |
| ATOM | 4245 | CB | ASN | A | 436 | 2.304 | 63.684 | −28.236 | 1.00 | 0.00 | C |
| ATOM | 4246 | CG | ASN | A | 436 | 1.272 | 62.670 | −28.711 | 1.00 | 0.00 | C |
| ATOM | 4247 | ND2 | ASN | A | 436 | 0.194 | 62.488 | −27.962 | 1.00 | 0.00 | N |
| ATOM | 4248 | OD1 | ASN | A | 436 | 1.464 | 62.018 | −29.732 | 1.00 | 0.00 | O |
| ATOM | 4249 | H | ASN | A | 436 | 2.116 | 63.426 | −25.674 | 1.00 | 0.00 | H |
| ATOM | 4250 | HA | ASN | A | 436 | 3.409 | 61.998 | −27.759 | 1.00 | 0.00 | H |
| ATOM | 4251 | 1HB | ASN | A | 436 | 1.796 | 64.457 | −27.660 | 1.00 | 0.00 | H |
| ATOM | 4252 | 2HB | ASN | A | 436 | 2.780 | 64.134 | −29.107 | 1.00 | 0.00 | H |
| ATOM | 4253 | 1HD2 | ASN | A | 436 | −0.512 | 61.823 | −28.246 | 1.00 | 0.00 | H |
| ATOM | 4254 | 2HD2 | ASN | A | 436 | 0.078 | 63.012 | −27.107 | 1.00 | 0.00 | H |
| ATOM | 4255 | N | ALA | A | 437 | 5.038 | 64.556 | −26.491 | 1.00 | 0.00 | N |
| ATOM | 4256 | CA | ALA | A | 437 | 6.305 | 65.234 | −26.371 | 1.00 | 0.00 | C |
| ATOM | 4257 | C | ALA | A | 437 | 7.431 | 64.266 | −25.982 | 1.00 | 0.00 | C |
| ATOM | 4258 | O | ALA | A | 437 | 8.570 | 64.712 | −25.811 | 1.00 | 0.00 | O |
| ATOM | 4259 | CB | ALA | A | 437 | 6.175 | 66.378 | −25.360 | 1.00 | 0.00 | C |
| ATOM | 4260 | H | ALA | A | 437 | 4.375 | 64.743 | −25.751 | 1.00 | 0.00 | H |
| ATOM | 4261 | HA | ALA | A | 437 | 6.531 | 65.751 | −27.303 | 1.00 | 0.00 | H |
| ATOM | 4262 | 1HB | ALA | A | 437 | 7.131 | 66.892 | −25.267 | 1.00 | 0.00 | H |
| ATOM | 4263 | 2HB | ALA | A | 437 | 5.416 | 67.082 | −25.703 | 1.00 | 0.00 | H |
| ATOM | 4264 | 3HB | ALA | A | 437 | 5.884 | 65.975 | −24.390 | 1.00 | 0.00 | H |
| ATOM | 4265 | N | THR | A | 438 | 7.162 | 62.963 | −25.855 | 1.00 | 0.00 | N |
| ATOM | 4266 | CA | THR | A | 438 | 8.188 | 61.939 | −25.727 | 1.00 | 0.00 | C |
| ATOM | 4267 | C | THR | A | 438 | 8.827 | 61.725 | −27.105 | 1.00 | 0.00 | C |
| ATOM | 4268 | O | THR | A | 438 | 8.636 | 60.713 | −27.774 | 1.00 | 0.00 | O |
| ATOM | 4269 | CB | THR | A | 438 | 7.588 | 60.659 | −25.124 | 1.00 | 0.00 | C |
| ATOM | 4270 | CG2 | THR | A | 438 | 8.672 | 59.665 | −24.706 | 1.00 | 0.00 | C |
| ATOM | 4271 | OG1 | THR | A | 438 | 6.847 | 60.991 | −23.966 | 1.00 | 0.00 | O |
| ATOM | 4272 | H | THR | A | 438 | 6.204 | 62.644 | −25.844 | 1.00 | 0.00 | H |
| ATOM | 4273 | HA | THR | A | 438 | 8.898 | 62.230 | −24.952 | 1.00 | 0.00 | H |
| ATOM | 4274 | HB | THR | A | 438 | 6.964 | 60.164 | −25.868 | 1.00 | 0.00 | H |
| ATOM | 4275 | 1HG2 | THR | A | 438 | 8.207 | 58.774 | −24.285 | 1.00 | 0.00 | H |
| ATOM | 4276 | 2HG2 | THR | A | 438 | 9.266 | 59.387 | −25.576 | 1.00 | 0.00 | H |
| ATOM | 4277 | 3HG2 | THR | A | 438 | 9.318 | 60.125 | −23.958 | 1.00 | 0.00 | H |
| ATOM | 4278 | HG1 | THR | A | 438 | 6.471 | 60.194 | −23.586 | 1.00 | 0.00 | H |
| ATOM | 4279 | N | GLY | A | 439 | 9.619 | 62.710 | −27.509 | 1.00 | 0.00 | N |
| ATOM | 4280 | CA | GLY | A | 439 | 10.309 | 62.808 | −28.776 | 1.00 | 0.00 | C |
| ATOM | 4281 | C | GLY | A | 439 | 11.101 | 64.111 | −28.688 | 1.00 | 0.00 | C |
| ATOM | 4282 | O | GLY | A | 439 | 11.209 | 64.674 | −27.600 | 1.00 | 0.00 | O |
| ATOM | 4283 | H | GLY | A | 439 | 9.791 | 63.500 | −26.904 | 1.00 | 0.00 | H |
| ATOM | 4284 | 1HA | GLY | A | 439 | 10.963 | 61.946 | −28.901 | 1.00 | 0.00 | H |
| ATOM | 4285 | 2HA | GLY | A | 439 | 9.581 | 62.832 | −29.586 | 1.00 | 0.00 | H |
| ATOM | 4286 | N | LYS | A | 440 | 11.635 | 64.635 | −29.791 | 1.00 | 0.00 | N |
| ATOM | 4287 | CA | LYS | A | 440 | 12.697 | 65.636 | −29.725 | 1.00 | 0.00 | C |
| ATOM | 4288 | C | LYS | A | 440 | 13.908 | 64.908 | −29.120 | 1.00 | 0.00 | C |
| ATOM | 4289 | O | LYS | A | 440 | 14.251 | 63.834 | −29.606 | 1.00 | 0.00 | O |
| ATOM | 4290 | CB | LYS | A | 440 | 12.272 | 66.917 | −28.958 | 1.00 | 0.00 | C |
| ATOM | 4291 | CG | LYS | A | 440 | 10.885 | 67.469 | −29.342 | 1.00 | 0.00 | C |
| ATOM | 4292 | CD | LYS | A | 440 | 10.365 | 68.490 | −28.308 | 1.00 | 0.00 | C |
| ATOM | 4293 | CE | LYS | A | 440 | 9.430 | 67.879 | −27.244 | 1.00 | 0.00 | C |
| ATOM | 4294 | NZ | LYS | A | 440 | 10.053 | 66.802 | −26.445 | 1.00 | 0.00 | N1+ |
| ATOM | 4295 | H | LYS | A | 440 | 11.307 | 64.340 | −30.700 | 1.00 | 0.00 | H |
| ATOM | 4296 | HA | LYS | A | 440 | 12.847 | 66.073 | −30.712 | 1.00 | 0.00 | H |
| ATOM | 4297 | 1HB | LYS | A | 440 | 12.241 | 66.707 | −27.889 | 1.00 | 0.00 | H |
| ATOM | 4298 | 2HB | LYS | A | 440 | 12.991 | 67.712 | −29.151 | 1.00 | 0.00 | H |
| ATOM | 4299 | 1HG | LYS | A | 440 | 10.947 | 67.966 | −30.310 | 1.00 | 0.00 | H |
| ATOM | 4300 | 2HG | LYS | A | 440 | 10.170 | 66.648 | −29.400 | 1.00 | 0.00 | H |
| ATOM | 4301 | 1HD | LYS | A | 440 | 11.208 | 68.935 | −27.779 | 1.00 | 0.00 | H |
| ATOM | 4302 | 2HD | LYS | A | 440 | 9.804 | 69.272 | −28.819 | 1.00 | 0.00 | H |
| ATOM | 4303 | 1HE | LYS | A | 440 | 9.115 | 68.655 | −26.547 | 1.00 | 0.00 | H |
| ATOM | 4304 | 2HE | LYS | A | 440 | 8.554 | 67.451 | −27.732 | 1.00 | 0.00 | H |
| ATOM | 4305 | 1HZ | LYS | A | 440 | 9.386 | 66.452 | −25.773 | 1.00 | 0.00 | H |
| ATOM | 4306 | 2HZ | LYS | A | 440 | 10.340 | 66.052 | −27.057 | 1.00 | 0.00 | H |
| ATOM | 4307 | 3HZ | LYS | A | 440 | 10.860 | 67.167 | −25.959 | 1.00 | 0.00 | H |
| ATOM | 4308 | N | ASP | A | 441 | 14.483 | 65.421 | −28.031 | 1.00 | 0.00 | N |
| ATOM | 4309 | CA | ASP | A | 441 | 15.262 | 64.628 | −27.093 | 1.00 | 0.00 | C |
| ATOM | 4310 | C | ASP | A | 441 | 14.521 | 64.753 | −25.762 | 1.00 | 0.00 | C |
| ATOM | 4311 | O | ASP | A | 441 | 14.618 | 65.778 | −25.087 | 1.00 | 0.00 | O |
| ATOM | 4312 | CB | ASP | A | 441 | 16.688 | 65.179 | −26.974 | 1.00 | 0.00 | C |
| ATOM | 4313 | CG | ASP | A | 441 | 17.401 | 64.563 | −25.784 | 1.00 | 0.00 | C |
| ATOM | 4314 | OD1 | ASP | A | 441 | 17.145 | 63.377 | −25.479 | 1.00 | 0.00 | O |
| ATOM | 4315 | OD2 | ASP | A | 441 | 18.159 | 65.280 | −25.093 | 1.00 | 0.00 | O1− |
| ATOM | 4316 | H | ASP | A | 441 | 14.387 | 66.406 | −27.826 | 1.00 | 0.00 | H |
| ATOM | 4317 | HA | ASP | A | 441 | 15.293 | 63.592 | −27.431 | 1.00 | 0.00 | H |
| ATOM | 4318 | 1HB | ASP | A | 441 | 17.246 | 64.944 | −27.880 | 1.00 | 0.00 | H |
| ATOM | 4319 | 2HB | ASP | A | 441 | 16.650 | 66.260 | −26.842 | 1.00 | 0.00 | H |
| ATOM | 4320 | N | SER | A | 442 | 13.718 | 63.755 | −25.405 | 1.00 | 0.00 | N |
| ATOM | 4321 | CA | SER | A | 442 | 12.900 | 63.753 | −24.206 | 1.00 | 0.00 | C |
| ATOM | 4322 | C | SER | A | 442 | 12.496 | 62.310 | −23.892 | 1.00 | 0.00 | C |
| ATOM | 4323 | O | SER | A | 442 | 12.218 | 61.535 | −24.804 | 1.00 | 0.00 | O |
| ATOM | 4324 | CB | SER | A | 442 | 11.648 | 64.628 | −24.388 | 1.00 | 0.00 | C |
| ATOM | 4325 | OG | SER | A | 442 | 11.959 | 65.952 | −24.816 | 1.00 | 0.00 | O |
| ATOM | 4326 | H | SER | A | 442 | 13.649 | 62.929 | −25.983 | 1.00 | 0.00 | H |
| ATOM | 4327 | HA | SER | A | 442 | 13.463 | 64.190 | −23.381 | 1.00 | 0.00 | H |
| ATOM | 4328 | 1HB | SER | A | 442 | 10.996 | 64.179 | −25.138 | 1.00 | 0.00 | H |
| ATOM | 4329 | 2HB | SER | A | 442 | 11.114 | 64.701 | −23.441 | 1.00 | 0.00 | H |
| ATOM | 4330 | HG | SER | A | 442 | 11.147 | 66.455 | −24.915 | 1.00 | 0.00 | H |
| ATOM | 4331 | N | LEU | A | 443 | 12.456 | 61.971 | −22.607 | 1.00 | 0.00 | N |
| ATOM | 4332 | CA | LEU | A | 443 | 11.981 | 60.706 | −22.064 | 1.00 | 0.00 | C |
| ATOM | 4333 | C | LEU | A | 443 | 11.259 | 61.098 | −20.778 | 1.00 | 0.00 | C |
| ATOM | 4334 | O | LEU | A | 443 | 11.648 | 62.100 | −20.171 | 1.00 | 0.00 | O |
| ATOM | 4335 | CB | LEU | A | 443 | 13.158 | 59.771 | −21.750 | 1.00 | 0.00 | C |
| ATOM | 4336 | CG | LEU | A | 443 | 13.902 | 59.244 | −22.988 | 1.00 | 0.00 | C |
| ATOM | 4337 | CD1 | LEU | A | 443 | 15.163 | 58.501 | −22.531 | 1.00 | 0.00 | C |
| ATOM | 4338 | CD2 | LEU | A | 443 | 13.029 | 58.294 | −23.818 | 1.00 | 0.00 | C |
| ATOM | 4339 | H | LEU | A | 443 | 12.776 | 62.620 | −21.903 | 1.00 | 0.00 | H |
| ATOM | 4340 | HA | LEU | A | 443 | 11.291 | 60.244 | −22.770 | 1.00 | 0.00 | H |
| ATOM | 4341 | 1HB | LEU | A | 443 | 13.891 | 60.300 | −21.141 | 1.00 | 0.00 | H |
| ATOM | 4342 | 2HB | LEU | A | 443 | 12.795 | 58.899 | −21.204 | 1.00 | 0.00 | H |
| ATOM | 4343 | HG | LEU | A | 443 | 14.167 | 60.080 | −23.635 | 1.00 | 0.00 | H |
| ATOM | 4344 | 1HD1 | LEU | A | 443 | 15.698 | 58.124 | −23.403 | 1.00 | 0.00 | H |
| ATOM | 4345 | 2HD1 | LEU | A | 443 | 15.808 | 59.184 | −21.978 | 1.00 | 0.00 | H |
| ATOM | 4346 | 3HD1 | LEU | A | 443 | 14.882 | 57.667 | −21.889 | 1.00 | 0.00 | H |
| ATOM | 4347 | 1HD2 | LEU | A | 443 | 13.593 | 57.945 | −24.683 | 1.00 | 0.00 | H |
| ATOM | 4348 | 2HD2 | LEU | A | 443 | 12.738 | 57.440 | −23.206 | 1.00 | 0.00 | H |
| ATOM | 4349 | 3HD2 | LEU | A | 443 | 12.136 | 58.821 | −24.155 | 1.00 | 0.00 | H |
| ATOM | 4350 | N | THR | A | 444 | 10.223 | 60.361 | −20.384 | 1.00 | 0.00 | N |
| ATOM | 4351 | CA | THR | A | 444 | 9.065 | 60.983 | −19.757 | 1.00 | 0.00 | C |
| ATOM | 4352 | C | THR | A | 444 | 8.552 | 60.149 | −18.575 | 1.00 | 0.00 | C |
| ATOM | 4353 | O | THR | A | 444 | 8.595 | 58.921 | −18.621 | 1.00 | 0.00 | O |
| ATOM | 4354 | CB | THR | A | 444 | 8.005 | 61.135 | −20.859 | 1.00 | 0.00 | C |
| ATOM | 4355 | CG2 | THR | A | 444 | 6.762 | 61.894 | −20.391 | 1.00 | 0.00 | C |
| ATOM | 4356 | OG1 | THR | A | 444 | 8.572 | 61.817 | −21.966 | 1.00 | 0.00 | O |
| ATOM | 4357 | H | THR | A | 444 | 10.235 | 59.360 | −20.518 | 1.00 | 0.00 | H |
| ATOM | 4358 | HA | THR | A | 444 | 9.326 | 61.989 | −19.428 | 1.00 | 0.00 | H |
| ATOM | 4359 | HB | THR | A | 444 | 7.703 | 60.149 | −21.212 | 1.00 | 0.00 | H |
| ATOM | 4360 | 1HG2 | THR | A | 444 | 6.050 | 61.969 | −21.213 | 1.00 | 0.00 | H |
| ATOM | 4361 | 2HG2 | THR | A | 444 | 6.301 | 61.361 | −19.559 | 1.00 | 0.00 | H |
| ATOM | 4362 | 3HG2 | THR | A | 444 | 7.048 | 62.895 | −20.066 | 1.00 | 0.00 | H |
| ATOM | 4363 | HG1 | THR | A | 444 | 7.911 | 61.914 | −22.656 | 1.00 | 0.00 | H |
| ATOM | 4364 | N | LEU | A | 445 | 8.060 | 60.800 | −17.518 | 1.00 | 0.00 | N |
| ATOM | 4365 | CA | LEU | A | 445 | 7.563 | 60.155 | −16.309 | 1.00 | 0.00 | C |
| ATOM | 4366 | C | LEU | A | 445 | 6.350 | 60.928 | −15.797 | 1.00 | 0.00 | C |
| ATOM | 4367 | O | LEU | A | 445 | 6.403 | 62.152 | −15.703 | 1.00 | 0.00 | O |
| ATOM | 4368 | CB | LEU | A | 445 | 8.679 | 60.166 | −15.251 | 1.00 | 0.00 | C |
| ATOM | 4369 | CG | LEU | A | 445 | 8.238 | 59.721 | −13.843 | 1.00 | 0.00 | C |
| ATOM | 4370 | CD1 | LEU | A | 445 | 7.713 | 58.285 | −13.833 | 1.00 | 0.00 | C |
| ATOM | 4371 | CD2 | LEU | A | 445 | 9.432 | 59.781 | −12.889 | 1.00 | 0.00 | C |
| ATOM | 4372 | H | LEU | A | 445 | 8.015 | 61.809 | −17.523 | 1.00 | 0.00 | H |
| ATOM | 4373 | HA | LEU | A | 445 | 7.273 | 59.129 | −16.538 | 1.00 | 0.00 | H |
| ATOM | 4374 | 1HB | LEU | A | 445 | 9.478 | 59.492 | −15.558 | 1.00 | 0.00 | H |
| ATOM | 4375 | 2HB | LEU | A | 445 | 9.075 | 61.177 | −15.151 | 1.00 | 0.00 | H |
| ATOM | 4376 | HG | LEU | A | 445 | 7.435 | 60.368 | −13.491 | 1.00 | 0.00 | H |
| ATOM | 4377 | 1HD1 | LEU | A | 445 | 7.414 | 58.014 | −12.820 | 1.00 | 0.00 | H |
| ATOM | 4378 | 2HD1 | LEU | A | 445 | 6.853 | 58.208 | −14.498 | 1.00 | 0.00 | H |
| ATOM | 4379 | 3HD1 | LEU | A | 445 | 8.497 | 57.609 | −14.173 | 1.00 | 0.00 | H |
| ATOM | 4380 | 1HD2 | LEU | A | 445 | 9.119 | 59.466 | −11.894 | 1.00 | 0.00 | H |
| ATOM | 4381 | 2HD2 | LEU | A | 445 | 10.219 | 59.119 | −13.248 | 1.00 | 0.00 | H |
| ATOM | 4382 | 3HD2 | LEU | A | 445 | 9.810 | 60.803 | −12.844 | 1.00 | 0.00 | H |
| ATOM | 4383 | N | VAL | A | 446 | 5.290 | 60.221 | −15.409 | 1.00 | 0.00 | N |
| ATOM | 4384 | CA | VAL | A | 446 | 4.163 | 60.798 | −14.699 | 1.00 | 0.00 | C |
| ATOM | 4385 | C | VAL | A | 446 | 4.340 | 60.480 | −13.208 | 1.00 | 0.00 | C |
| ATOM | 4386 | O | VAL | A | 446 | 4.668 | 59.346 | −12.850 | 1.00 | 0.00 | O |
| ATOM | 4387 | CB | VAL | A | 446 | 2.839 | 60.245 | −15.253 | 1.00 | 0.00 | C |
| ATOM | 4388 | CG1 | VAL | A | 446 | 1.645 | 60.969 | −14.618 | 1.00 | 0.00 | C |
| ATOM | 4389 | CG2 | VAL | A | 446 | 2.755 | 60.392 | −16.779 | 1.00 | 0.00 | C |
| ATOM | 4390 | H | VAL | A | 446 | 5.244 | 59.232 | −15.606 | 1.00 | 0.00 | H |
| ATOM | 4391 | HA | VAL | A | 446 | 4.118 | 61.869 | −14.895 | 1.00 | 0.00 | H |
| ATOM | 4392 | HB | VAL | A | 446 | 2.779 | 59.176 | −15.053 | 1.00 | 0.00 | H |
| ATOM | 4393 | 1HG1 | VAL | A | 446 | 0.717 | 60.565 | −15.023 | 1.00 | 0.00 | H |
| ATOM | 4394 | 2HG1 | VAL | A | 446 | 1.663 | 60.824 | −13.538 | 1.00 | 0.00 | H |
| ATOM | 4395 | 3HG1 | VAL | A | 446 | 1.704 | 62.034 | −14.842 | 1.00 | 0.00 | H |
| ATOM | 4396 | 1HG2 | VAL | A | 446 | 1.805 | 59.990 | −17.132 | 1.00 | 0.00 | H |
| ATOM | 4397 | 2HG2 | VAL | A | 446 | 2.825 | 61.445 | −17.048 | 1.00 | 0.00 | H |
| ATOM | 4398 | 3HG2 | VAL | A | 446 | 3.575 | 59.843 | −17.243 | 1.00 | 0.00 | H |
| ATOM | 4399 | N | PHE | A | 447 | 4.123 | 61.470 | −12.344 | 1.00 | 0.00 | N |
| ATOM | 4400 | CA | PHE | A | 447 | 4.295 | 61.357 | −10.901 | 1.00 | 0.00 | C |
| ATOM | 4401 | C | PHE | A | 447 | 2.914 | 61.436 | −10.252 | 1.00 | 0.00 | C |
| ATOM | 4402 | O | PHE | A | 447 | 2.200 | 62.413 | −10.473 | 1.00 | 0.00 | O |
| ATOM | 4403 | CB | PHE | A | 447 | 5.188 | 62.506 | −10.397 | 1.00 | 0.00 | C |
| ATOM | 4404 | CG | PHE | A | 447 | 6.470 | 62.065 | −9.721 | 1.00 | 0.00 | C |
| ATOM | 4405 | CD1 | PHE | A | 447 | 6.471 | 61.706 | −8.360 | 1.00 | 0.00 | C |
| ATOM | 4406 | CD2 | PHE | A | 447 | 7.682 | 62.020 | −10.437 | 1.00 | 0.00 | C |
| ATOM | 4407 | CE1 | PHE | A | 447 | 7.665 | 61.336 | −7.718 | 1.00 | 0.00 | C |
| ATOM | 4408 | CE2 | PHE | A | 447 | 8.877 | 61.639 | −9.802 | 1.00 | 0.00 | C |
| ATOM | 4409 | CZ | PHE | A | 447 | 8.870 | 61.296 | −8.440 | 1.00 | 0.00 | C |
| ATOM | 4410 | H | PHE | A | 447 | 3.817 | 62.373 | −12.677 | 1.00 | 0.00 | H |
| ATOM | 4411 | HA | PHE | A | 447 | 4.770 | 60.405 | −10.665 | 1.00 | 0.00 | H |
| ATOM | 4412 | 1HB | PHE | A | 447 | 5.477 | 63.138 | −11.236 | 1.00 | 0.00 | H |
| ATOM | 4413 | 2HB | PHE | A | 447 | 4.638 | 63.100 | −9.667 | 1.00 | 0.00 | H |
| ATOM | 4414 | HD1 | PHE | A | 447 | 5.547 | 61.713 | −7.800 | 1.00 | 0.00 | H |
| ATOM | 4415 | HD2 | PHE | A | 447 | 7.700 | 62.281 | −11.485 | 1.00 | 0.00 | H |
| ATOM | 4416 | HE1 | PHE | A | 447 | 7.657 | 61.083 | −6.668 | 1.00 | 0.00 | H |
| ATOM | 4417 | HE2 | PHE | A | 447 | 9.800 | 61.610 | −10.362 | 1.00 | 0.00 | H |
| ATOM | 4418 | HZ | PHE | A | 447 | 9.785 | 61.002 | −7.948 | 1.00 | 0.00 | H |
| ATOM | 4419 | N | VAL | A | 448 | 2.562 | 60.443 | −9.439 | 1.00 | 0.00 | N |
| ATOM | 4420 | CA | VAL | A | 448 | 1.281 | 60.345 | −8.748 | 1.00 | 0.00 | C |
| ATOM | 4421 | C | VAL | A | 448 | 1.549 | 60.052 | −7.266 | 1.00 | 0.00 | C |
| ATOM | 4422 | O | VAL | A | 448 | 2.687 | 59.784 | −6.874 | 1.00 | 0.00 | O |
| ATOM | 4423 | CB | VAL | A | 448 | 0.420 | 59.217 | −9.374 | 1.00 | 0.00 | C |
| ATOM | 4424 | CG1 | VAL | A | 448 | −0.268 | 59.650 | −10.667 | 1.00 | 0.00 | C |
| ATOM | 4425 | CG2 | VAL | A | 448 | 1.202 | 57.934 | −9.670 | 1.00 | 0.00 | C |
| ATOM | 4426 | H | VAL | A | 448 | 3.204 | 59.683 | −9.263 | 1.00 | 0.00 | H |
| ATOM | 4427 | HA | VAL | A | 448 | 0.734 | 61.281 | −8.862 | 1.00 | 0.00 | H |
| ATOM | 4428 | HB | VAL | A | 448 | −0.321 | 58.880 | −8.648 | 1.00 | 0.00 | H |
| ATOM | 4429 | 1HG1 | VAL | A | 448 | −0.857 | 58.821 | −11.061 | 1.00 | 0.00 | H |
| ATOM | 4430 | 2HG1 | VAL | A | 448 | −0.924 | 60.496 | −10.465 | 1.00 | 0.00 | H |
| ATOM | 4431 | 3HG1 | VAL | A | 448 | 0.484 | 59.940 | −11.400 | 1.00 | 0.00 | H |
| ATOM | 4432 | 1HG2 | VAL | A | 448 | 0.532 | 57.192 | −10.106 | 1.00 | 0.00 | H |
| ATOM | 4433 | 2HG2 | VAL | A | 448 | 2.007 | 58.152 | −10.372 | 1.00 | 0.00 | H |
| ATOM | 4434 | 3HG2 | VAL | A | 448 | 1.623 | 57.542 | −8.744 | 1.00 | 0.00 | H |
| ATOM | 4435 | N | GLU | A | 449 | 0.492 | 60.018 | −6.459 | 1.00 | 0.00 | N |
| ATOM | 4436 | CA | GLU | A | 449 | 0.311 | 59.045 | −5.392 | 1.00 | 0.00 | C |
| ATOM | 4437 | C | GLU | A | 449 | −1.149 | 58.545 | −5.416 | 1.00 | 0.00 | C |
| ATOM | 4438 | O | GLU | A | 449 | −1.543 | 57.717 | −4.595 | 1.00 | 0.00 | O |
| ATOM | 4439 | CB | GLU | A | 449 | 0.749 | 59.636 | −4.039 | 1.00 | 0.00 | C |
| ATOM | 4440 | CG | GLU | A | 449 | 0.873 | 58.614 | −2.889 | 1.00 | 0.00 | C |
| ATOM | 4441 | CD | GLU | A | 449 | 1.811 | 57.463 | −3.213 | 1.00 | 0.00 | C |
| ATOM | 4442 | OE1 | GLU | A | 449 | 2.921 | 57.746 | −3.702 | 1.00 | 0.00 | O |
| ATOM | 4443 | OE2 | GLU | A | 449 | 1.423 | 56.294 | −2.994 | 1.00 | 0.00 | O1− |
| ATOM | 4444 | H | GLU | A | 449 | −0.248 | 60.697 | −6.567 | 1.00 | 0.00 | H |
| ATOM | 4445 | HA | GLU | A | 449 | 1.061 | 58.260 | −5.484 | 1.00 | 0.00 | H |
| ATOM | 4446 | 1HB | GLU | A | 449 | 1.727 | 60.105 | −4.146 | 1.00 | 0.00 | H |
| ATOM | 4447 | 2HB | GLU | A | 449 | 0.022 | 60.382 | −3.716 | 1.00 | 0.00 | H |
| ATOM | 4448 | 1HG | GLU | A | 449 | 1.258 | 59.112 | −1.999 | 1.00 | 0.00 | H |
| ATOM | 4449 | 2HG | GLU | A | 449 | −0.107 | 58.189 | −2.672 | 1.00 | 0.00 | H |
| ATOM | 4450 | N | THR | A | 450 | −1.978 | 59.007 | −6.359 | 1.00 | 0.00 | N |
| ATOM | 4451 | CA | THR | A | 450 | −3.322 | 58.492 | −6.533 | 1.00 | 0.00 | C |
| ATOM | 4452 | C | THR | A | 450 | −3.259 | 56.972 | −6.730 | 1.00 | 0.00 | C |
| ATOM | 4453 | O | THR | A | 450 | −2.750 | 56.472 | −7.734 | 1.00 | 0.00 | O |
| ATOM | 4454 | CB | THR | A | 450 | −4.009 | 59.189 | −7.720 | 1.00 | 0.00 | C |
| ATOM | 4455 | CG2 | THR | A | 450 | −4.771 | 60.427 | −7.257 | 1.00 | 0.00 | C |
| ATOM | 4456 | OG1 | THR | A | 450 | −3.077 | 59.569 | −8.713 | 1.00 | 0.00 | O |
| ATOM | 4457 | H | THR | A | 450 | −1.674 | 59.742 | −6.982 | 1.00 | 0.00 | H |
| ATOM | 4458 | HA | THR | A | 450 | −3.931 | 58.767 | −5.672 | 1.00 | 0.00 | H |
| ATOM | 4459 | HB | THR | A | 450 | −4.689 | 58.491 | −8.207 | 1.00 | 0.00 | H |
| ATOM | 4460 | 1HG2 | THR | A | 450 | −5.248 | 60.902 | −8.114 | 1.00 | 0.00 | H |
| ATOM | 4461 | 2HG2 | THR | A | 450 | −5.532 | 60.136 | −6.533 | 1.00 | 0.00 | H |
| ATOM | 4462 | 3HG2 | THR | A | 450 | −4.078 | 61.129 | −6.793 | 1.00 | 0.00 | H |
| ATOM | 4463 | HG1 | THR | A | 450 | −3.537 | 60.000 | −9.437 | 1.00 | 0.00 | H |
| ATOM | 4464 | N | LYS | A | 451 | −3.785 | 56.208 | −5.771 | 1.00 | 0.00 | N |
| ATOM | 4465 | CA | LYS | A | 451 | −3.362 | 54.828 | −5.607 | 1.00 | 0.00 | C |
| ATOM | 4466 | C | LYS | A | 451 | −3.864 | 53.919 | −6.730 | 1.00 | 0.00 | C |
| ATOM | 4467 | O | LYS | A | 451 | −3.424 | 52.775 | −6.829 | 1.00 | 0.00 | O |
| ATOM | 4468 | CB | LYS | A | 451 | −3.783 | 54.309 | −4.219 | 1.00 | 0.00 | C |
| ATOM | 4469 | CG | LYS | A | 451 | −3.128 | 55.071 | −3.049 | 1.00 | 0.00 | C |
| ATOM | 4470 | CD | LYS | A | 451 | −1.804 | 54.467 | −2.552 | 1.00 | 0.00 | C |
| ATOM | 4471 | CE | LYS | A | 451 | −0.777 | 54.253 | −3.671 | 1.00 | 0.00 | C |
| ATOM | 4472 | NZ | LYS | A | 451 | 0.533 | 53.852 | −3.146 | 1.00 | 0.00 | N1+ |
| ATOM | 4473 | H | LYS | A | 451 | −4.484 | 56.589 | −5.149 | 1.00 | 0.00 | H |
| ATOM | 4474 | HA | LYS | A | 451 | −2.278 | 54.789 | −5.500 | 1.00 | 0.00 | H |
| ATOM | 4475 | 1HB | LYS | A | 451 | −4.863 | 54.407 | −4.107 | 1.00 | 0.00 | H |
| ATOM | 4476 | 2HB | LYS | A | 451 | −3.501 | 53.261 | −4.122 | 1.00 | 0.00 | H |
| ATOM | 4477 | 1HG | LYS | A | 451 | −2.913 | 56.094 | −3.356 | 1.00 | 0.00 | H |
| ATOM | 4478 | 2HG | LYS | A | 451 | −3.808 | 55.083 | −2.197 | 1.00 | 0.00 | H |
| ATOM | 4479 | 1HD | LYS | A | 451 | −1.353 | 55.133 | −1.817 | 1.00 | 0.00 | H |
| ATOM | 4480 | 2HD | LYS | A | 451 | −1.995 | 53.497 | −2.094 | 1.00 | 0.00 | H |
| ATOM | 4481 | 1HE | LYS | A | 451 | −1.128 | 53.469 | −4.342 | 1.00 | 0.00 | H |
| ATOM | 4482 | 2HE | LYS | A | 451 | −0.650 | 55.179 | −4.230 | 1.00 | 0.00 | H |
| ATOM | 4483 | 1HZ | LYS | A | 451 | 1.178 | 53.722 | −3.913 | 1.00 | 0.00 | H |
| ATOM | 4484 | 2HZ | LYS | A | 451 | 0.884 | 54.571 | −2.530 | 1.00 | 0.00 | H |
| ATOM | 4485 | 3HZ | LYS | A | 451 | 0.441 | 52.986 | −2.634 | 1.00 | 0.00 | H |
| ATOM | 4486 | N | LYS | A | 452 | −4.782 | 54.405 | −7.568 | 1.00 | 0.00 | N |
| ATOM | 4487 | CA | LYS | A | 452 | −5.131 | 53.768 | −8.826 | 1.00 | 0.00 | C |
| ATOM | 4488 | C | LYS | A | 452 | −5.291 | 54.822 | −9.924 | 1.00 | 0.00 | C |
| ATOM | 4489 | O | LYS | A | 452 | −5.910 | 54.556 | −10.949 | 1.00 | 0.00 | O |
| ATOM | 4490 | CB | LYS | A | 452 | −6.375 | 52.865 | −8.663 | 1.00 | 0.00 | C |
| ATOM | 4491 | CG | LYS | A | 452 | −6.146 | 51.618 | −7.783 | 1.00 | 0.00 | C |
| ATOM | 4492 | CD | LYS | A | 452 | −5.234 | 50.608 | −8.501 | 1.00 | 0.00 | C |
| ATOM | 4493 | CE | LYS | A | 452 | −4.471 | 49.642 | −7.584 | 1.00 | 0.00 | C |
| ATOM | 4494 | NZ | LYS | A | 452 | −3.472 | 48.879 | −8.363 | 1.00 | 0.00 | N1+ |
| ATOM | 4495 | H | LYS | A | 452 | −5.273 | 55.257 | −7.339 | 1.00 | 0.00 | H |
| ATOM | 4496 | HA | LYS | A | 452 | −4.400 | 52.993 | −9.056 | 1.00 | 0.00 | H |
| ATOM | 4497 | 1HB | LYS | A | 452 | −7.180 | 53.436 | −8.201 | 1.00 | 0.00 | H |
| ATOM | 4498 | 2HB | LYS | A | 452 | −6.696 | 52.509 | −9.641 | 1.00 | 0.00 | H |
| ATOM | 4499 | 1HG | LYS | A | 452 | −5.674 | 51.915 | −6.846 | 1.00 | 0.00 | H |
| ATOM | 4500 | 2HG | LYS | A | 452 | −7.103 | 51.141 | −7.572 | 1.00 | 0.00 | H |
| ATOM | 4501 | 1HD | LYS | A | 452 | −5.832 | 49.990 | −9.170 | 1.00 | 0.00 | H |
| ATOM | 4502 | 2HD | LYS | A | 452 | −4.481 | 51.145 | −9.078 | 1.00 | 0.00 | H |
| ATOM | 4503 | 1HE | LYS | A | 452 | −3.958 | 50.208 | −6.806 | 1.00 | 0.00 | H |
| ATOM | 4504 | 2HE | LYS | A | 452 | −5.172 | 48.946 | −7.125 | 1.00 | 0.00 | H |
| ATOM | 4505 | 1HZ | LYS | A | 452 | −2.977 | 48.247 | −7.749 | 1.00 | 0.00 | H |
| ATOM | 4506 | 2HZ | LYS | A | 452 | −3.940 | 48.349 | −9.084 | 1.00 | 0.00 | H |
| ATOM | 4507 | 3HZ | LYS | A | 452 | −2.816 | 49.518 | −8.788 | 1.00 | 0.00 | H |
| ATOM | 4508 | N | GLY | A | 453 | −4.649 | 55.981 | −9.744 | 1.00 | 0.00 | N |
| ATOM | 4509 | CA | GLY | A | 453 | −4.277 | 56.846 | −10.861 | 1.00 | 0.00 | C |
| ATOM | 4510 | C | GLY | A | 453 | −3.001 | 56.285 | −11.500 | 1.00 | 0.00 | C |
| ATOM | 4511 | O | GLY | A | 453 | −2.503 | 56.793 | −12.497 | 1.00 | 0.00 | O |
| ATOM | 4512 | H | GLY | A | 453 | −4.406 | 56.285 | −8.812 | 1.00 | 0.00 | H |
| ATOM | 4513 | 1HA | GLY | A | 453 | −5.084 | 56.863 | −11.593 | 1.00 | 0.00 | H |
| ATOM | 4514 | 2HA | GLY | A | 453 | −4.098 | 57.857 | −10.495 | 1.00 | 0.00 | H |
| ATOM | 4515 | N | ALA | A | 454 | −2.468 | 55.217 | −10.901 | 1.00 | 0.00 | N |
| ATOM | 4516 | CA | ALA | A | 454 | −1.509 | 54.327 | −11.511 | 1.00 | 0.00 | C |
| ATOM | 4517 | C | ALA | A | 454 | −2.253 | 53.488 | −12.557 | 1.00 | 0.00 | C |
| ATOM | 4518 | O | ALA | A | 454 | −2.229 | 53.813 | −13.741 | 1.00 | 0.00 | O |
| ATOM | 4519 | CB | ALA | A | 454 | −0.853 | 53.498 | −10.401 | 1.00 | 0.00 | C |
| ATOM | 4520 | H | ALA | A | 454 | −2.731 | 54.986 | −9.954 | 1.00 | 0.00 | H |
| ATOM | 4521 | HA | ALA | A | 454 | −0.688 | 54.908 | −11.930 | 1.00 | 0.00 | H |
| ATOM | 4522 | 1HB | ALA | A | 454 | −0.123 | 52.817 | −10.838 | 1.00 | 0.00 | H |
| ATOM | 4523 | 2HB | ALA | A | 454 | −0.351 | 54.164 | −9.698 | 1.00 | 0.00 | H |
| ATOM | 4524 | 3HB | ALA | A | 454 | −1.616 | 52.924 | −9.875 | 1.00 | 0.00 | H |
| ATOM | 4525 | N | ASP | A | 455 | −2.925 | 52.421 | −12.115 | 1.00 | 0.00 | N |
| ATOM | 4526 | CA | ASP | A | 455 | −3.662 | 51.492 | −12.959 | 1.00 | 0.00 | C |
| ATOM | 4527 | C | ASP | A | 455 | −4.556 | 52.236 | −13.952 | 1.00 | 0.00 | C |
| ATOM | 4528 | O | ASP | A | 455 | −4.500 | 51.978 | −15.153 | 1.00 | 0.00 | O |
| ATOM | 4529 | CB | ASP | A | 455 | −4.538 | 50.562 | −12.105 | 1.00 | 0.00 | C |
| ATOM | 4530 | CG | ASP | A | 455 | −3.776 | 49.668 | −11.148 | 1.00 | 0.00 | C |
| ATOM | 4531 | OD1 | ASP | A | 455 | −2.746 | 50.106 | −10.587 | 1.00 | 0.00 | O |
| ATOM | 4532 | OD2 | ASP | A | 455 | −4.286 | 48.583 | −10.803 | 1.00 | 0.00 | O1− |
| ATOM | 4533 | H | ASP | A | 455 | −2.945 | 52.210 | −11.128 | 1.00 | 0.00 | H |
| ATOM | 4534 | HA | ASP | A | 455 | −2.960 | 50.862 | −13.506 | 1.00 | 0.00 | H |
| ATOM | 4535 | 1HB | ASP | A | 455 | −5.219 | 51.159 | −11.499 | 1.00 | 0.00 | H |
| ATOM | 4536 | 2HB | ASP | A | 455 | −5.113 | 49.904 | −12.756 | 1.00 | 0.00 | H |
| ATOM | 4537 | N | SER | A | 456 | −5.399 | 53.152 | −13.463 | 1.00 | 0.00 | N |
| ATOM | 4538 | CA | SER | A | 456 | −6.357 | 53.853 | −14.305 | 1.00 | 0.00 | C |
| ATOM | 4539 | C | SER | A | 456 | −5.667 | 55.038 | −14.989 | 1.00 | 0.00 | C |
| ATOM | 4540 | O | SER | A | 456 | −6.150 | 56.172 | −14.963 | 1.00 | 0.00 | O |
| ATOM | 4541 | CB | SER | A | 456 | −7.593 | 54.271 | −13.492 | 1.00 | 0.00 | C |
| ATOM | 4542 | OG | SER | A | 456 | −8.716 | 54.478 | −14.336 | 1.00 | 0.00 | O |
| ATOM | 4543 | H | SER | A | 456 | −5.385 | 53.376 | −12.478 | 1.00 | 0.00 | H |
| ATOM | 4544 | HA | SER | A | 456 | −6.809 | 53.150 | −15.004 | 1.00 | 0.00 | H |
| ATOM | 4545 | 1HB | SER | A | 456 | −7.838 | 53.488 | −12.773 | 1.00 | 0.00 | H |
| ATOM | 4546 | 2HB | SER | A | 456 | −7.382 | 55.199 | −12.960 | 1.00 | 0.00 | H |
| ATOM | 4547 | HG | SER | A | 456 | −9.473 | 54.737 | −13.805 | 1.00 | 0.00 | H |
| ATOM | 4548 | N | LEU | A | 457 | −4.563 | 54.744 | −15.663 | 1.00 | 0.00 | N |
| ATOM | 4549 | CA | LEU | A | 457 | −3.757 | 55.662 | −16.439 | 1.00 | 0.00 | C |
| ATOM | 4550 | C | LEU | A | 457 | −2.752 | 54.798 | −17.192 | 1.00 | 0.00 | C |
| ATOM | 4551 | O | LEU | A | 457 | −2.489 | 55.031 | −18.367 | 1.00 | 0.00 | O |
| ATOM | 4552 | CB | LEU | A | 457 | −3.066 | 56.711 | −15.554 | 1.00 | 0.00 | C |
| ATOM | 4553 | CG | LEU | A | 457 | −2.551 | 57.921 | −16.354 | 1.00 | 0.00 | C |
| ATOM | 4554 | CD1 | LEU | A | 457 | −3.704 | 58.805 | −16.851 | 1.00 | 0.00 | C |
| ATOM | 4555 | CD2 | LEU | A | 457 | −1.645 | 58.771 | −15.456 | 1.00 | 0.00 | C |
| ATOM | 4556 | H | LEU | A | 457 | −4.209 | 53.798 | −15.663 | 1.00 | 0.00 | H |
| ATOM | 4557 | HA | LEU | A | 457 | −4.402 | 56.244 | −17.099 | 1.00 | 0.00 | H |
| ATOM | 4558 | 1HB | LEU | A | 457 | −3.772 | 57.081 | −14.810 | 1.00 | 0.00 | H |
| ATOM | 4559 | 2HB | LEU | A | 457 | −2.212 | 56.258 | −15.051 | 1.00 | 0.00 | H |
| ATOM | 4560 | HG | LEU | A | 457 | −1.991 | 57.571 | −17.221 | 1.00 | 0.00 | H |
| ATOM | 4561 | 1HD1 | LEU | A | 457 | −3.301 | 59.648 | −17.411 | 1.00 | 0.00 | H |
| ATOM | 4562 | 2HD1 | LEU | A | 457 | −4.358 | 58.219 | −17.498 | 1.00 | 0.00 | H |
| ATOM | 4563 | 3HD1 | LEU | A | 457 | −4.274 | 59.174 | −15.999 | 1.00 | 0.00 | H |
| ATOM | 4564 | 1HD2 | LEU | A | 457 | −1.278 | 59.629 | −16.019 | 1.00 | 0.00 | H |
| ATOM | 4565 | 2HD2 | LEU | A | 457 | −2.212 | 59.119 | −14.593 | 1.00 | 0.00 | H |
| ATOM | 4566 | 3HD2 | LEU | A | 457 | −0.801 | 58.171 | −15.118 | 1.00 | 0.00 | H |
| ATOM | 4567 | N | GLU | A | 458 | −2.269 | 53.731 | −16.548 | 1.00 | 0.00 | N |
| ATOM | 4568 | CA | GLU | A | 458 | −1.681 | 52.601 | −17.249 | 1.00 | 0.00 | C |
| ATOM | 4569 | C | GLU | A | 458 | −2.637 | 52.182 | −18.371 | 1.00 | 0.00 | C |
| ATOM | 4570 | O | GLU | A | 458 | −2.231 | 52.128 | −19.527 | 1.00 | 0.00 | O |
| ATOM | 4571 | CB | GLU | A | 458 | −1.390 | 51.457 | −16.266 | 1.00 | 0.00 | C |
| ATOM | 4572 | CG | GLU | A | 458 | −0.229 | 50.565 | −16.735 | 1.00 | 0.00 | C |
| ATOM | 4573 | CD | GLU | A | 458 | 0.221 | 49.607 | −15.646 | 1.00 | 0.00 | C |
| ATOM | 4574 | OE1 | GLU | A | 458 | −0.527 | 49.465 | −14.664 | 1.00 | 0.00 | O |
| ATOM | 4575 | OE2 | GLU | A | 458 | 1.375 | 49.127 | −15.664 | 1.00 | 0.00 | O1− |
| ATOM | 4576 | H | GLU | A | 458 | −2.305 | 53.690 | −15.539 | 1.00 | 0.00 | H |
| ATOM | 4577 | HA | GLU | A | 458 | −0.691 | 52.874 | −17.614 | 1.00 | 0.00 | H |
| ATOM | 4578 | 1HB | GLU | A | 458 | −1.124 | 51.871 | −15.293 | 1.00 | 0.00 | H |
| ATOM | 4579 | 2HB | GLU | A | 458 | −2.276 | 50.831 | −16.163 | 1.00 | 0.00 | H |
| ATOM | 4580 | 1HG | GLU | A | 458 | −0.548 | 49.977 | −17.597 | 1.00 | 0.00 | H |
| ATOM | 4581 | 2HG | GLU | A | 458 | 0.619 | 51.189 | −17.015 | 1.00 | 0.00 | H |
| ATOM | 4582 | N | ASP | A | 459 | −3.900 | 51.952 | −17.994 | 1.00 | 0.00 | N |
| ATOM | 4583 | CA | ASP | A | 459 | −5.090 | 51.924 | −18.843 | 1.00 | 0.00 | C |
| ATOM | 4584 | C | ASP | A | 459 | −4.945 | 52.833 | −20.070 | 1.00 | 0.00 | C |
| ATOM | 4585 | O | ASP | A | 459 | −4.784 | 52.364 | −21.196 | 1.00 | 0.00 | O |
| ATOM | 4586 | CB | ASP | A | 459 | −6.284 | 52.352 | −17.967 | 1.00 | 0.00 | C |
| ATOM | 4587 | CG | ASP | A | 459 | −7.476 | 52.870 | −18.752 | 1.00 | 0.00 | C |
| ATOM | 4588 | OD1 | ASP | A | 459 | −7.836 | 52.240 | −19.761 | 1.00 | 0.00 | O |
| ATOM | 4589 | OD2 | ASP | A | 459 | −8.001 | 53.926 | −18.335 | 1.00 | 0.00 | O1− |
| ATOM | 4590 | H | ASP | A | 459 | −4.112 | 51.772 | −17.023 | 1.00 | 0.00 | H |
| ATOM | 4591 | HA | ASP | A | 459 | −5.287 | 50.900 | −19.158 | 1.00 | 0.00 | H |
| ATOM | 4592 | 1HB | ASP | A | 459 | −6.631 | 51.499 | −17.384 | 1.00 | 0.00 | H |
| ATOM | 4593 | 2HB | ASP | A | 459 | −5.973 | 53.150 | −17.293 | 1.00 | 0.00 | H |
| ATOM | 4594 | N | PHE | A | 460 | −5.040 | 54.142 | −19.833 | 1.00 | 0.00 | N |
| ATOM | 4595 | CA | PHE | A | 460 | −5.060 | 55.163 | −20.867 | 1.00 | 0.00 | C |
| ATOM | 4596 | C | PHE | A | 460 | −3.870 | 54.988 | −21.807 | 1.00 | 0.00 | C |
| ATOM | 4597 | O | PHE | A | 460 | −3.995 | 55.080 | −23.022 | 1.00 | 0.00 | O |
| ATOM | 4598 | CB | PHE | A | 460 | −5.011 | 56.534 | −20.176 | 1.00 | 0.00 | C |
| ATOM | 4599 | CG | PHE | A | 460 | −5.111 | 57.772 | −21.049 | 1.00 | 0.00 | C |
| ATOM | 4600 | CD1 | PHE | A | 460 | −6.027 | 57.849 | −22.117 | 1.00 | 0.00 | C |
| ATOM | 4601 | CD2 | PHE | A | 460 | −4.294 | 58.888 | −20.776 | 1.00 | 0.00 | C |
| ATOM | 4602 | CE1 | PHE | A | 460 | −6.132 | 59.023 | −22.884 | 1.00 | 0.00 | C |
| ATOM | 4603 | CE2 | PHE | A | 460 | −4.391 | 60.059 | −21.548 | 1.00 | 0.00 | C |
| ATOM | 4604 | CZ | PHE | A | 460 | −5.318 | 60.130 | −22.598 | 1.00 | 0.00 | C |
| ATOM | 4605 | H | PHE | A | 460 | −5.104 | 54.480 | −18.884 | 1.00 | 0.00 | H |
| ATOM | 4606 | HA | PHE | A | 460 | −5.991 | 55.092 | −21.430 | 1.00 | 0.00 | H |
| ATOM | 4607 | 1HB | PHE | A | 460 | −5.839 | 56.617 | −19.472 | 1.00 | 0.00 | H |
| ATOM | 4608 | 2HB | PHE | A | 460 | −4.068 | 56.639 | −19.641 | 1.00 | 0.00 | H |
| ATOM | 4609 | HD1 | PHE | A | 460 | −6.654 | 57.003 | −22.353 | 1.00 | 0.00 | H |
| ATOM | 4610 | HD2 | PHE | A | 460 | −3.581 | 58.851 | −19.965 | 1.00 | 0.00 | H |
| ATOM | 4611 | HE1 | PHE | A | 460 | −6.842 | 59.075 | −23.697 | 1.00 | 0.00 | H |
| ATOM | 4612 | HE2 | PHE | A | 460 | −3.751 | 60.902 | −21.333 | 1.00 | 0.00 | H |
| ATOM | 4613 | HZ | PHE | A | 460 | −5.408 | 61.033 | −23.184 | 1.00 | 0.00 | H |
| ATOM | 4614 | N | LEU | A | 461 | −2.699 | 54.749 | −21.227 | 1.00 | 0.00 | N |
| ATOM | 4615 | CA | LEU | A | 461 | −1.445 | 54.748 | −21.956 | 1.00 | 0.00 | C |
| ATOM | 4616 | C | LEU | A | 461 | −1.102 | 53.331 | −22.422 | 1.00 | 0.00 | C |
| ATOM | 4617 | O | LEU | A | 461 | 0.032 | 53.041 | −22.797 | 1.00 | 0.00 | O |
| ATOM | 4618 | CB | LEU | A | 461 | −0.363 | 55.374 | −21.067 | 1.00 | 0.00 | C |
| ATOM | 4619 | CG | LEU | A | 461 | −0.762 | 56.755 | −20.513 | 1.00 | 0.00 | C |
| ATOM | 4620 | CD1 | LEU | A | 461 | 0.343 | 57.265 | −19.584 | 1.00 | 0.00 | C |
| ATOM | 4621 | CD2 | LEU | A | 461 | −1.064 | 57.767 | −21.623 | 1.00 | 0.00 | C |
| ATOM | 4622 | H | LEU | A | 461 | −2.655 | 54.557 | −20.236 | 1.00 | 0.00 | H |
| ATOM | 4623 | HA | LEU | A | 461 | −1.502 | 55.462 | −22.778 | 1.00 | 0.00 | H |
| ATOM | 4624 | 1HB | LEU | A | 461 | −0.168 | 54.721 | −20.217 | 1.00 | 0.00 | H |
| ATOM | 4625 | 2HB | LEU | A | 461 | 0.553 | 55.502 | −21.645 | 1.00 | 0.00 | H |
| ATOM | 4626 | HG | LEU | A | 461 | −1.730 | 56.683 | −20.018 | 1.00 | 0.00 | H |
| ATOM | 4627 | 1HD1 | LEU | A | 461 | 0.065 | 58.242 | −19.190 | 1.00 | 0.00 | H |
| ATOM | 4628 | 2HD1 | LEU | A | 461 | 0.477 | 56.565 | −18.759 | 1.00 | 0.00 | H |
| ATOM | 4629 | 3HD1 | LEU | A | 461 | 1.276 | 57.351 | −20.142 | 1.00 | 0.00 | H |
| ATOM | 4630 | 1HD2 | LEU | A | 461 | −1.340 | 58.723 | −21.178 | 1.00 | 0.00 | H |
| ATOM | 4631 | 2HD2 | LEU | A | 461 | −0.179 | 57.898 | −22.246 | 1.00 | 0.00 | H |
| ATOM | 4632 | 3HD2 | LEU | A | 461 | −1.888 | 57.400 | −22.235 | 1.00 | 0.00 | H |
| ATOM | 4633 | N | TYR | A | 462 | −2.091 | 52.443 | −22.400 | 1.00 | 0.00 | N |
| ATOM | 4634 | CA | TYR | A | 462 | −2.044 | 51.114 | −22.968 | 1.00 | 0.00 | C |
| ATOM | 4635 | C | TYR | A | 462 | −3.052 | 51.114 | −24.110 | 1.00 | 0.00 | C |
| ATOM | 4636 | O | TYR | A | 462 | −2.687 | 50.852 | −25.255 | 1.00 | 0.00 | O |
| ATOM | 4637 | CB | TYR | A | 462 | −2.371 | 50.065 | −21.894 | 1.00 | 0.00 | C |
| ATOM | 4638 | CG | TYR | A | 462 | −2.837 | 48.728 | −22.434 | 1.00 | 0.00 | C |
| ATOM | 4639 | CD1 | TYR | A | 462 | −1.924 | 47.809 | −22.982 | 1.00 | 0.00 | C |
| ATOM | 4640 | CD2 | TYR | A | 462 | −4.203 | 48.392 | −22.407 | 1.00 | 0.00 | C |
| ATOM | 4641 | CE1 | TYR | A | 462 | −2.372 | 46.579 | −23.497 | 1.00 | 0.00 | C |
| ATOM | 4642 | CE2 | TYR | A | 462 | −4.652 | 47.167 | −22.923 | 1.00 | 0.00 | C |
| ATOM | 4643 | CZ | TYR | A | 462 | −3.739 | 46.248 | −23.474 | 1.00 | 0.00 | C |
| ATOM | 4644 | OH | TYR | A | 462 | −4.171 | 45.056 | −23.981 | 1.00 | 0.00 | O |
| ATOM | 4645 | H | TYR | A | 462 | −2.969 | 52.675 | −21.959 | 1.00 | 0.00 | H |
| ATOM | 4646 | HA | TYR | A | 462 | −1.029 | 50.895 | −23.298 | 1.00 | 0.00 | H |
| ATOM | 4647 | 1HB | TYR | A | 462 | −1.483 | 49.869 | −21.294 | 1.00 | 0.00 | H |
| ATOM | 4648 | 2HB | TYR | A | 462 | −3.168 | 50.439 | −21.251 | 1.00 | 0.00 | H |
| ATOM | 4649 | HD1 | TYR | A | 462 | −0.870 | 48.045 | −23.009 | 1.00 | 0.00 | H |
| ATOM | 4650 | HD2 | TYR | A | 462 | −4.920 | 49.080 | −21.985 | 1.00 | 0.00 | H |
| ATOM | 4651 | HE1 | TYR | A | 462 | −1.664 | 45.879 | −23.915 | 1.00 | 0.00 | H |
| ATOM | 4652 | HE2 | TYR | A | 462 | −5.704 | 46.924 | −22.900 | 1.00 | 0.00 | H |
| ATOM | 4653 | HH | TYR | A | 462 | −5.125 | 44.994 | −23.885 | 1.00 | 0.00 | H |
| ATOM | 4654 | N | HIS | A | 463 | −4.310 | 51.429 | −23.798 | 1.00 | 0.00 | N |
| ATOM | 4655 | CA | HIS | A | 463 | −5.386 | 51.479 | −24.773 | 1.00 | 0.00 | C |
| ATOM | 4656 | C | HIS | A | 463 | −5.042 | 52.449 | −25.899 | 1.00 | 0.00 | C |
| ATOM | 4657 | O | HIS | A | 463 | −5.084 | 52.062 | −27.067 | 1.00 | 0.00 | O |
| ATOM | 4658 | CB | HIS | A | 463 | −6.719 | 51.805 | −24.088 | 1.00 | 0.00 | C |
| ATOM | 4659 | CG | HIS | A | 463 | −7.237 | 50.637 | −23.282 | 1.00 | 0.00 | C |
| ATOM | 4660 | CD2 | HIS | A | 463 | −7.484 | 49.333 | −23.636 | 1.00 | 0.00 | C |
| ATOM | 4661 | ND1 | HIS | A | 463 | −7.565 | 50.709 | −21.933 | 1.00 | 0.00 | N |
| ATOM | 4662 | CE1 | HIS | A | 463 | −7.964 | 49.493 | −21.533 | 1.00 | 0.00 | C |
| ATOM | 4663 | NE2 | HIS | A | 463 | −7.926 | 48.613 | −22.535 | 1.00 | 0.00 | N |
| ATOM | 4664 | H | HIS | A | 463 | −4.553 | 51.648 | −22.843 | 1.00 | 0.00 | H |
| ATOM | 4665 | HA | HIS | A | 463 | −5.610 | 50.470 | −25.119 | 1.00 | 0.00 | H |
| ATOM | 4666 | 1HB | HIS | A | 463 | −6.584 | 52.652 | −23.414 | 1.00 | 0.00 | H |
| ATOM | 4667 | 2HB | HIS | A | 463 | −7.464 | 52.057 | −24.842 | 1.00 | 0.00 | H |
| ATOM | 4668 | HD2 | HIS | A | 463 | −7.325 | 49.010 | −24.654 | 1.00 | 0.00 | H |
| ATOM | 4669 | HD1 | HIS | A | 463 | −7.478 | 51.585 | −21.438 | 1.00 | 0.00 | H |
| ATOM | 4670 | HE1 | HIS | A | 463 | −8.262 | 49.332 | −20.508 | 1.00 | 0.00 | H |
| ATOM | 4671 | N | GLU | A | 464 | −4.618 | 53.668 | −25.560 | 1.00 | 0.00 | N |
| ATOM | 4672 | CA | GLU | A | 464 | −4.324 | 54.679 | −26.562 | 1.00 | 0.00 | C |
| ATOM | 4673 | C | GLU | A | 464 | −2.860 | 54.564 | −27.017 | 1.00 | 0.00 | C |
| ATOM | 4674 | O | GLU | A | 464 | −2.301 | 55.479 | −27.617 | 1.00 | 0.00 | O |
| ATOM | 4675 | CB | GLU | A | 464 | −4.662 | 56.074 | −26.017 | 1.00 | 0.00 | C |
| ATOM | 4676 | CG | GLU | A | 464 | −6.063 | 56.143 | −25.386 | 1.00 | 0.00 | C |
| ATOM | 4677 | CD | GLU | A | 464 | −7.140 | 55.619 | −26.320 | 1.00 | 0.00 | C |
| ATOM | 4678 | OE1 | GLU | A | 464 | −7.215 | 56.160 | −27.443 | 1.00 | 0.00 | O |
| ATOM | 4679 | OE2 | GLU | A | 464 | −7.860 | 54.693 | −25.891 | 1.00 | 0.00 | O1− |
| ATOM | 4680 | H | GLU | A | 464 | −4.493 | 53.907 | −24.586 | 1.00 | 0.00 | H |
| ATOM | 4681 | HA | GLU | A | 464 | −5.032 | 54.591 | −27.386 | 1.00 | 0.00 | H |
| ATOM | 4682 | 1HB | GLU | A | 464 | −3.938 | 56.351 | −25.251 | 1.00 | 0.00 | H |
| ATOM | 4683 | 2HB | GLU | A | 464 | −4.627 | 56.800 | −26.829 | 1.00 | 0.00 | H |
| ATOM | 4684 | 1HG | GLU | A | 464 | −6.083 | 55.543 | −24.476 | 1.00 | 0.00 | H |
| ATOM | 4685 | 2HG | GLU | A | 464 | −6.302 | 57.179 | −25.142 | 1.00 | 0.00 | H |
| ATOM | 4686 | N | GLY | A | 465 | −2.206 | 53.434 | −26.733 | 1.00 | 0.00 | N |
| ATOM | 4687 | CA | GLY | A | 465 | −1.033 | 53.008 | −27.475 | 1.00 | 0.00 | C |
| ATOM | 4688 | C | GLY | A | 465 | 0.279 | 53.604 | −26.960 | 1.00 | 0.00 | C |
| ATOM | 4689 | O | GLY | A | 465 | 1.353 | 53.215 | −27.426 | 1.00 | 0.00 | O |
| ATOM | 4690 | H | GLY | A | 465 | −2.524 | 52.842 | −25.979 | 1.00 | 0.00 | H |
| ATOM | 4691 | 1HA | GLY | A | 465 | −0.938 | 51.924 | −27.415 | 1.00 | 0.00 | H |
| ATOM | 4692 | 2HA | GLY | A | 465 | −1.134 | 53.307 | −28.518 | 1.00 | 0.00 | H |
| ATOM | 4693 | N | TYR | A | 466 | 0.230 | 54.512 | −25.984 | 1.00 | 0.00 | N |
| ATOM | 4694 | CA | TYR | A | 466 | 1.402 | 55.229 | −25.495 | 1.00 | 0.00 | C |
| ATOM | 4695 | C | TYR | A | 466 | 2.226 | 54.356 | −24.537 | 1.00 | 0.00 | C |
| ATOM | 4696 | O | TYR | A | 466 | 2.428 | 54.725 | −23.383 | 1.00 | 0.00 | O |
| ATOM | 4697 | CB | TYR | A | 466 | 0.969 | 56.532 | −24.809 | 1.00 | 0.00 | C |
| ATOM | 4698 | CG | TYR | A | 466 | 0.240 | 57.520 | −25.695 | 1.00 | 0.00 | C |
| ATOM | 4699 | CD1 | TYR | A | 466 | 0.947 | 58.289 | −26.638 | 1.00 | 0.00 | C |
| ATOM | 4700 | CD2 | TYR | A | 466 | −1.151 | 57.692 | −25.586 | 1.00 | 0.00 | C |
| ATOM | 4701 | CE1 | TYR | A | 466 | 0.274 | 59.210 | −27.458 | 1.00 | 0.00 | C |
| ATOM | 4702 | CE2 | TYR | A | 466 | −1.826 | 58.611 | −26.407 | 1.00 | 0.00 | C |
| ATOM | 4703 | CZ | TYR | A | 466 | −1.117 | 59.382 | −27.349 | 1.00 | 0.00 | C |
| ATOM | 4704 | OH | TYR | A | 466 | −1.767 | 60.278 | −28.150 | 1.00 | 0.00 | O |
| ATOM | 4705 | H | TYR | A | 466 | −0.652 | 54.732 | −25.544 | 1.00 | 0.00 | H |
| ATOM | 4706 | HA | TYR | A | 466 | 2.006 | 55.559 | −26.341 | 1.00 | 0.00 | H |
| ATOM | 4707 | 1HB | TYR | A | 466 | 0.295 | 56.302 | −23.984 | 1.00 | 0.00 | H |
| ATOM | 4708 | 2HB | TYR | A | 466 | 1.848 | 57.051 | −24.426 | 1.00 | 0.00 | H |
| ATOM | 4709 | HD1 | TYR | A | 466 | 2.016 | 58.174 | −26.735 | 1.00 | 0.00 | H |
| ATOM | 4710 | HD2 | TYR | A | 466 | −1.712 | 57.115 | −24.866 | 1.00 | 0.00 | H |
| ATOM | 4711 | HE1 | TYR | A | 466 | 0.825 | 59.793 | −28.181 | 1.00 | 0.00 | H |
| ATOM | 4712 | HE2 | TYR | A | 466 | −2.896 | 58.731 | −26.319 | 1.00 | 0.00 | H |
| ATOM | 4713 | HH | TYR | A | 466 | −2.705 | 60.269 | −27.946 | 1.00 | 0.00 | H |
| ATOM | 4714 | N | ALA | A | 467 | 2.728 | 53.229 | −25.058 | 1.00 | 0.00 | N |
| ATOM | 4715 | CA | ALA | A | 467 | 3.366 | 52.121 | −24.358 | 1.00 | 0.00 | C |
| ATOM | 4716 | C | ALA | A | 467 | 4.007 | 52.505 | −23.019 | 1.00 | 0.00 | C |
| ATOM | 4717 | O | ALA | A | 467 | 4.943 | 53.305 | −22.978 | 1.00 | 0.00 | O |
| ATOM | 4718 | CB | ALA | A | 467 | 4.408 | 51.485 | −25.281 | 1.00 | 0.00 | C |
| ATOM | 4719 | H | ALA | A | 467 | 2.688 | 53.072 | −26.055 | 1.00 | 0.00 | H |
| ATOM | 4720 | HA | ALA | A | 467 | 2.641 | 51.321 | −24.206 | 1.00 | 0.00 | H |
| ATOM | 4721 | 1HB | ALA | A | 467 | 4.892 | 50.655 | −24.766 | 1.00 | 0.00 | H |
| ATOM | 4722 | 2HB | ALA | A | 467 | 3.920 | 51.118 | −26.183 | 1.00 | 0.00 | H |
| ATOM | 4723 | 3HB | ALA | A | 467 | 5.157 | 52.229 | −25.551 | 1.00 | 0.00 | H |
| ATOM | 4724 | N | CYS | A | 468 | 3.560 | 51.864 | −21.938 | 1.00 | 0.00 | N |
| ATOM | 4725 | CA | CYS | A | 468 | 3.615 | 52.423 | −20.596 | 1.00 | 0.00 | C |
| ATOM | 4726 | C | CYS | A | 468 | 3.917 | 51.334 | −19.566 | 1.00 | 0.00 | C |
| ATOM | 4727 | O | CYS | A | 468 | 4.158 | 50.178 | −19.920 | 1.00 | 0.00 | O |
| ATOM | 4728 | CB | CYS | A | 468 | 2.238 | 53.040 | −20.294 | 1.00 | 0.00 | C |
| ATOM | 4729 | SG | CYS | A | 468 | 0.984 | 51.726 | −20.134 | 1.00 | 0.00 | S |
| ATOM | 4730 | H | CYS | A | 468 | 3.158 | 50.942 | −22.028 | 1.00 | 0.00 | H |
| ATOM | 4731 | HA | CYS | A | 468 | 4.408 | 53.170 | −20.545 | 1.00 | 0.00 | H |
| ATOM | 4732 | 1HB | CYS | A | 468 | 2.288 | 53.602 | −19.361 | 1.00 | 0.00 | H |
| ATOM | 4733 | 2HB | CYS | A | 468 | 1.953 | 53.709 | −21.106 | 1.00 | 0.00 | H |
| ATOM | 4734 | HG | CYS | A | 468 | 0.004 | 52.178 | −19.918 | 1.00 | 0.00 | H |
| ATOM | 4735 | N | THR | A | 469 | 3.844 | 51.712 | −18.294 | 1.00 | 0.00 | N |
| ATOM | 4736 | CA | THR | A | 469 | 3.462 | 50.874 | −17.171 | 1.00 | 0.00 | C |
| ATOM | 4737 | C | THR | A | 469 | 3.290 | 51.835 | −15.983 | 1.00 | 0.00 | C |
| ATOM | 4738 | O | THR | A | 469 | 3.779 | 52.969 | −16.040 | 1.00 | 0.00 | O |
| ATOM | 4739 | CB | THR | A | 469 | 4.455 | 49.725 | −16.905 | 1.00 | 0.00 | C |
| ATOM | 4740 | CG2 | THR | A | 469 | 5.917 | 50.182 | −16.875 | 1.00 | 0.00 | C |
| ATOM | 4741 | OG1 | THR | A | 469 | 4.142 | 49.070 | −15.688 | 1.00 | 0.00 | O |
| ATOM | 4742 | H | THR | A | 469 | 4.066 | 52.663 | −18.036 | 1.00 | 0.00 | H |
| ATOM | 4743 | HA | THR | A | 469 | 2.581 | 50.289 | −17.437 | 1.00 | 0.00 | H |
| ATOM | 4744 | HB | THR | A | 469 | 4.323 | 48.948 | −17.658 | 1.00 | 0.00 | H |
| ATOM | 4745 | 1HG2 | THR | A | 469 | 6.562 | 49.325 | −16.683 | 1.00 | 0.00 | H |
| ATOM | 4746 | 2HG2 | THR | A | 469 | 6.179 | 50.626 | −17.835 | 1.00 | 0.00 | H |
| ATOM | 4747 | 3HG2 | THR | A | 469 | 6.051 | 50.922 | −16.085 | 1.00 | 0.00 | H |
| ATOM | 4748 | HG1 | THR | A | 469 | 4.769 | 48.359 | −15.537 | 1.00 | 0.00 | H |
| ATOM | 4749 | N | SER | A | 470 | 2.613 | 51.412 | −14.916 | 1.00 | 0.00 | N |
| ATOM | 4750 | CA | SER | A | 470 | 2.489 | 52.166 | −13.690 | 1.00 | 0.00 | C |
| ATOM | 4751 | C | SER | A | 470 | 3.122 | 51.352 | −12.561 | 1.00 | 0.00 | C |
| ATOM | 4752 | O | SER | A | 470 | 2.524 | 50.445 | −11.981 | 1.00 | 0.00 | O |
| ATOM | 4753 | CB | SER | A | 470 | 1.035 | 52.597 | −13.440 | 1.00 | 0.00 | C |
| ATOM | 4754 | OG | SER | A | 470 | 0.166 | 51.550 | −13.051 | 1.00 | 0.00 | O |
| ATOM | 4755 | H | SER | A | 470 | 2.149 | 50.516 | −14.934 | 1.00 | 0.00 | H |
| ATOM | 4756 | HA | SER | A | 470 | 2.888 | 53.169 | −13.838 | 1.00 | 0.00 | H |
| ATOM | 4757 | 1HB | SER | A | 470 | 1.008 | 53.340 | −12.643 | 1.00 | 0.00 | H |
| ATOM | 4758 | 2HB | SER | A | 470 | 0.621 | 53.027 | −14.352 | 1.00 | 0.00 | H |
| ATOM | 4759 | HG | SER | A | 470 | −0.717 | 51.901 | −12.916 | 1.00 | 0.00 | H |
| ATOM | 4760 | N | ILE | A | 471 | 4.369 | 51.676 | −12.228 | 1.00 | 0.00 | N |
| ATOM | 4761 | CA | ILE | A | 471 | 5.107 | 50.901 | −11.256 | 1.00 | 0.00 | C |
| ATOM | 4762 | C | ILE | A | 471 | 4.841 | 51.577 | −9.912 | 1.00 | 0.00 | C |
| ATOM | 4763 | O | ILE | A | 471 | 5.703 | 52.258 | −9.351 | 1.00 | 0.00 | O |
| ATOM | 4764 | CB | ILE | A | 471 | 6.594 | 50.791 | −11.649 | 1.00 | 0.00 | C |
| ATOM | 4765 | CG1 | ILE | A | 471 | 6.733 | 50.293 | −13.101 | 1.00 | 0.00 | C |
| ATOM | 4766 | CG2 | ILE | A | 471 | 7.298 | 49.790 | −10.723 | 1.00 | 0.00 | C |
| ATOM | 4767 | CD1 | ILE | A | 471 | 8.194 | 50.177 | −13.552 | 1.00 | 0.00 | C |
| ATOM | 4768 | H | ILE | A | 471 | 4.815 | 52.474 | −12.657 | 1.00 | 0.00 | H |
| ATOM | 4769 | HA | ILE | A | 471 | 4.814 | 49.854 | −11.327 | 1.00 | 0.00 | H |
| ATOM | 4770 | HB | ILE | A | 471 | 7.064 | 51.771 | −11.573 | 1.00 | 0.00 | H |
| ATOM | 4771 | 1HG1 | ILE | A | 471 | 6.278 | 49.307 | −13.194 | 1.00 | 0.00 | H |
| ATOM | 4772 | 2HG1 | ILE | A | 471 | 6.232 | 50.989 | −13.774 | 1.00 | 0.00 | H |
| ATOM | 4773 | 1HG2 | ILE | A | 471 | 8.348 | 49.712 | −11.002 | 1.00 | 0.00 | H |
| ATOM | 4774 | 2HG2 | ILE | A | 471 | 7.220 | 50.132 | −9.691 | 1.00 | 0.00 | H |
| ATOM | 4775 | 3HG2 | ILE | A | 471 | 6.824 | 48.812 | −10.817 | 1.00 | 0.00 | H |
| ATOM | 4776 | 1HD1 | ILE | A | 471 | 8.230 | 49.823 | −14.582 | 1.00 | 0.00 | H |
| ATOM | 4777 | 2HD1 | ILE | A | 471 | 8.673 | 51.154 | −13.487 | 1.00 | 0.00 | H |
| ATOM | 4778 | 3HD1 | ILE | A | 471 | 8.719 | 49.472 | −12.907 | 1.00 | 0.00 | H |
| ATOM | 4779 | N | HIS | A | 472 | 3.628 | 51.388 | −9.391 | 1.00 | 0.00 | N |
| ATOM | 4780 | CA | HIS | A | 472 | 3.264 | 51.944 | −8.099 | 1.00 | 0.00 | C |
| ATOM | 4781 | C | HIS | A | 472 | 3.937 | 51.144 | −6.977 | 1.00 | 0.00 | C |
| ATOM | 4782 | O | HIS | A | 472 | 4.408 | 50.024 | −7.190 | 1.00 | 0.00 | O |
| ATOM | 4783 | CB | HIS | A | 472 | 1.750 | 52.157 | −7.940 | 1.00 | 0.00 | C |
| ATOM | 4784 | CG | HIS | A | 472 | 0.832 | 50.964 | −7.947 | 1.00 | 0.00 | C |
| ATOM | 4785 | CD2 | HIS | A | 472 | 0.121 | 50.344 | −8.944 | 1.00 | 0.00 | C |
| ATOM | 4786 | ND1 | HIS | A | 472 | 0.479 | 50.298 | −6.785 | 1.00 | 0.00 | N |
| ATOM | 4787 | CE1 | HIS | A | 472 | −0.351 | 49.295 | −7.107 | 1.00 | 0.00 | C |
| ATOM | 4788 | NE2 | HIS | A | 472 | −0.614 | 49.287 | −8.413 | 1.00 | 0.00 | N |
| ATOM | 4789 | H | HIS | A | 472 | 2.940 | 50.848 | −9.896 | 1.00 | 0.00 | H |
| ATOM | 4790 | HA | HIS | A | 472 | 3.416 | 53.023 | −8.111 | 1.00 | 0.00 | H |
| ATOM | 4791 | 1HB | HIS | A | 472 | 1.549 | 52.646 | −6.986 | 1.00 | 0.00 | H |
| ATOM | 4792 | 2HB | HIS | A | 472 | 1.383 | 52.785 | −8.753 | 1.00 | 0.00 | H |
| ATOM | 4793 | HD2 | HIS | A | 472 | 0.191 | 50.702 | −9.960 | 1.00 | 0.00 | H |
| ATOM | 4794 | HD1 | HIS | A | 472 | 0.848 | 50.604 | −5.895 | 1.00 | 0.00 | H |
| ATOM | 4795 | HE1 | HIS | A | 472 | −0.714 | 48.626 | −6.341 | 1.00 | 0.00 | H |
| ATOM | 4796 | N | GLY | A | 473 | 4.107 | 51.749 | −5.802 | 1.00 | 0.00 | N |
| ATOM | 4797 | CA | GLY | A | 473 | 4.813 | 51.157 | −4.672 | 1.00 | 0.00 | C |
| ATOM | 4798 | C | GLY | A | 473 | 4.172 | 49.851 | −4.221 | 1.00 | 0.00 | C |
| ATOM | 4799 | O | GLY | A | 473 | 4.828 | 49.010 | −3.615 | 1.00 | 0.00 | O |
| ATOM | 4800 | H | GLY | A | 473 | 3.735 | 52.677 | −5.654 | 1.00 | 0.00 | H |
| ATOM | 4801 | 1HA | GLY | A | 473 | 5.845 | 50.950 | −4.956 | 1.00 | 0.00 | H |
| ATOM | 4802 | 2HA | GLY | A | 473 | 4.800 | 51.851 | −3.831 | 1.00 | 0.00 | H |
| ATOM | 4803 | N | ASP | A | 474 | 2.889 | 49.717 | −4.527 | 1.00 | 0.00 | N |
| ATOM | 4804 | CA | ASP | A | 474 | 1.987 | 48.697 | −4.034 | 1.00 | 0.00 | C |
| ATOM | 4805 | C | ASP | A | 474 | 1.796 | 47.640 | −5.135 | 1.00 | 0.00 | C |
| ATOM | 4806 | O | ASP | A | 474 | 0.888 | 46.814 | −5.068 | 1.00 | 0.00 | O |
| ATOM | 4807 | CB | ASP | A | 474 | 0.650 | 49.341 | −3.581 | 1.00 | 0.00 | C |
| ATOM | 4808 | CG | ASP | A | 474 | 0.660 | 50.861 | −3.438 | 1.00 | 0.00 | C |
| ATOM | 4809 | OD1 | ASP | A | 474 | 0.964 | 51.546 | −4.445 | 1.00 | 0.00 | O |
| ATOM | 4810 | OD2 | ASP | A | 474 | 0.358 | 51.376 | −2.344 | 1.00 | 0.00 | O1− |
| ATOM | 4811 | H | ASP | A | 474 | 2.451 | 50.368 | −5.163 | 1.00 | 0.00 | H |
| ATOM | 4812 | HA | ASP | A | 474 | 2.379 | 48.281 | −3.106 | 1.00 | 0.00 | H |
| ATOM | 4813 | 1HB | ASP | A | 474 | −0.130 | 49.109 | −4.306 | 1.00 | 0.00 | H |
| ATOM | 4814 | 2HB | ASP | A | 474 | 0.367 | 48.945 | −2.606 | 1.00 | 0.00 | H |
| ATOM | 4815 | N | ARG | A | 475 | 2.637 | 47.681 | −6.174 | 1.00 | 0.00 | N |
| ATOM | 4816 | CA | ARG | A | 475 | 2.785 | 46.584 | −7.124 | 1.00 | 0.00 | C |
| ATOM | 4817 | C | ARG | A | 475 | 3.590 | 45.475 | −6.443 | 1.00 | 0.00 | C |
| ATOM | 4818 | O | ARG | A | 475 | 4.359 | 45.734 | −5.517 | 1.00 | 0.00 | O |
| ATOM | 4819 | CB | ARG | A | 475 | 3.502 | 47.062 | −8.397 | 1.00 | 0.00 | C |
| ATOM | 4820 | CG | ARG | A | 475 | 2.610 | 47.917 | −9.301 | 1.00 | 0.00 | C |
| ATOM | 4821 | CD | ARG | A | 475 | 1.688 | 47.071 | −10.199 | 1.00 | 0.00 | C |
| ATOM | 4822 | NE | ARG | A | 475 | 0.550 | 47.873 | −10.681 | 1.00 | 0.00 | N |
| ATOM | 4823 | CZ | ARG | A | 475 | 0.258 | 48.272 | −11.930 | 1.00 | 0.00 | C |
| ATOM | 4824 | NH1 | ARG | A | 475 | 1.048 | 47.975 | −12.962 | 1.00 | 0.00 | N1+ |
| ATOM | 4825 | NH2 | ARG | A | 475 | −0.852 | 48.965 | −12.135 | 1.00 | 0.00 | N |
| ATOM | 4826 | H | ARG | A | 475 | 3.208 | 48.499 | −6.330 | 1.00 | 0.00 | H |
| ATOM | 4827 | HA | ARG | A | 475 | 1.800 | 46.226 | −7.422 | 1.00 | 0.00 | H |
| ATOM | 4828 | 1HB | ARG | A | 475 | 4.368 | 47.665 | −8.122 | 1.00 | 0.00 | H |
| ATOM | 4829 | 2HB | ARG | A | 475 | 3.830 | 46.199 | −8.976 | 1.00 | 0.00 | H |
| ATOM | 4830 | 1HG | ARG | A | 475 | 1.980 | 48.560 | −8.688 | 1.00 | 0.00 | H |
| ATOM | 4831 | 2HG | ARG | A | 475 | 3.234 | 48.532 | −9.950 | 1.00 | 0.00 | H |
| ATOM | 4832 | 1HD | ARG | A | 475 | 2.251 | 46.707 | −11.059 | 1.00 | 0.00 | H |
| ATOM | 4833 | 2HD | ARG | A | 475 | 1.305 | 46.223 | −9.631 | 1.00 | 0.00 | H |
| ATOM | 4834 | HE | ARG | A | 475 | −0.058 | 48.126 | −9.915 | 1.00 | 0.00 | H |
| ATOM | 4835 | 1HH1 | ARG | A | 475 | 0.804 | 48.288 | −13.891 | 1.00 | 0.00 | H |
| ATOM | 4836 | 2HH1 | ARG | A | 475 | 1.890 | 47.437 | −12.815 | 1.00 | 0.00 | H |
| ATOM | 4837 | 1HH2 | ARG | A | 475 | −1.090 | 49.274 | −13.066 | 1.00 | 0.00 | H |
| ATOM | 4838 | 2HH2 | ARG | A | 475 | −1.461 | 49.184 | −11.359 | 1.00 | 0.00 | H |
| ATOM | 4839 | N | SER | A | 476 | 3.431 | 44.229 | −6.883 | 1.00 | 0.00 | N |
| ATOM | 4840 | CA | SER | A | 476 | 3.995 | 43.116 | −6.154 | 1.00 | 0.00 | C |
| ATOM | 4841 | C | SER | A | 476 | 5.525 | 43.084 | −6.260 | 1.00 | 0.00 | C |
| ATOM | 4842 | O | SER | A | 476 | 6.128 | 43.551 | −7.230 | 1.00 | 0.00 | O |
| ATOM | 4843 | CB | SER | A | 476 | 3.358 | 41.795 | −6.612 | 1.00 | 0.00 | C |
| ATOM | 4844 | OG | SER | A | 476 | 3.563 | 41.548 | −7.990 | 1.00 | 0.00 | O |
| ATOM | 4845 | H | SER | A | 476 | 2.914 | 44.057 | −7.734 | 1.00 | 0.00 | H |
| ATOM | 4846 | HA | SER | A | 476 | 3.647 | 43.144 | −5.121 | 1.00 | 0.00 | H |
| ATOM | 4847 | 1HB | SER | A | 476 | 3.794 | 40.967 | −6.053 | 1.00 | 0.00 | H |
| ATOM | 4848 | 2HB | SER | A | 476 | 2.283 | 41.827 | −6.432 | 1.00 | 0.00 | H |
| ATOM | 4849 | HG | SER | A | 476 | 3.151 | 40.716 | −8.232 | 1.00 | 0.00 | H |
| ATOM | 4850 | N | GLN | A | 477 | 6.152 | 42.443 | −5.273 | 1.00 | 0.00 | N |
| ATOM | 4851 | CA | GLN | A | 477 | 7.557 | 42.080 | −5.292 | 1.00 | 0.00 | C |
| ATOM | 4852 | C | GLN | A | 477 | 7.710 | 40.933 | −6.302 | 1.00 | 0.00 | C |
| ATOM | 4853 | O | GLN | A | 477 | 7.962 | 39.789 | −5.929 | 1.00 | 0.00 | O |
| ATOM | 4854 | CB | GLN | A | 477 | 7.937 | 41.740 | −3.836 | 1.00 | 0.00 | C |
| ATOM | 4855 | CG | GLN | A | 477 | 9.285 | 41.060 | −3.580 | 1.00 | 0.00 | C |
| ATOM | 4856 | CD | GLN | A | 477 | 10.422 | 41.656 | −4.389 | 1.00 | 0.00 | C |
| ATOM | 4857 | NE2 | GLN | A | 477 | 10.700 | 41.058 | −5.534 | 1.00 | 0.00 | N |
| ATOM | 4858 | OE1 | GLN | A | 477 | 11.043 | 42.629 | −3.976 | 1.00 | 0.00 | O |
| ATOM | 4859 | H | GLN | A | 477 | 5.644 | 42.177 | −4.442 | 1.00 | 0.00 | H |
| ATOM | 4860 | HA | GLN | A | 477 | 8.157 | 42.960 | −5.525 | 1.00 | 0.00 | H |
| ATOM | 4861 | 1HB | GLN | A | 477 | 7.967 | 42.655 | −3.244 | 1.00 | 0.00 | H |
| ATOM | 4862 | 2HB | GLN | A | 477 | 7.195 | 41.060 | −3.416 | 1.00 | 0.00 | H |
| ATOM | 4863 | 1HG | GLN | A | 477 | 9.547 | 41.157 | −2.526 | 1.00 | 0.00 | H |
| ATOM | 4864 | 2HG | GLN | A | 477 | 9.215 | 40.004 | −3.840 | 1.00 | 0.00 | H |
| ATOM | 4865 | 1HE2 | GLN | A | 477 | 11.446 | 41.408 | −6.117 | 1.00 | 0.00 | H |
| ATOM | 4866 | 2HE2 | GLN | A | 477 | 10.167 | 40.252 | −5.825 | 1.00 | 0.00 | H |
| ATOM | 4867 | N | ARG | A | 478 | 7.553 | 41.265 | −7.583 | 1.00 | 0.00 | N |
| ATOM | 4868 | CA | ARG | A | 478 | 7.415 | 40.394 | −8.740 | 1.00 | 0.00 | C |
| ATOM | 4869 | C | ARG | A | 478 | 7.160 | 41.342 | −9.919 | 1.00 | 0.00 | C |
| ATOM | 4870 | O | ARG | A | 478 | 8.015 | 41.495 | −10.789 | 1.00 | 0.00 | O |
| ATOM | 4871 | CB | ARG | A | 478 | 6.270 | 39.381 | −8.544 | 1.00 | 0.00 | C |
| ATOM | 4872 | CG | ARG | A | 478 | 6.285 | 38.240 | −9.571 | 1.00 | 0.00 | C |
| ATOM | 4873 | CD | ARG | A | 478 | 4.950 | 38.108 | −10.316 | 1.00 | 0.00 | C |
| ATOM | 4874 | NE | ARG | A | 478 | 4.865 | 39.085 | −11.409 | 1.00 | 0.00 | N |
| ATOM | 4875 | CZ | ARG | A | 478 | 4.031 | 40.121 | −11.552 | 1.00 | 0.00 | C |
| ATOM | 4876 | NH1 | ARG | A | 478 | 3.187 | 40.457 | −10.580 | 1.00 | 0.00 | N1+ |
| ATOM | 4877 | NH2 | ARG | A | 478 | 4.042 | 40.838 | −12.664 | 1.00 | 0.00 | N |
| ATOM | 4878 | H | ARG | A | 478 | 7.517 | 42.238 | −7.852 | 1.00 | 0.00 | H |
| ATOM | 4879 | HA | ARG | A | 478 | 8.321 | 39.799 | −8.858 | 1.00 | 0.00 | H |
| ATOM | 4880 | 1HB | ARG | A | 478 | 6.347 | 38.932 | −7.554 | 1.00 | 0.00 | H |
| ATOM | 4881 | 2HB | ARG | A | 478 | 5.312 | 39.892 | −8.637 | 1.00 | 0.00 | H |
| ATOM | 4882 | 1HG | ARG | A | 478 | 7.066 | 38.425 | −10.309 | 1.00 | 0.00 | H |
| ATOM | 4883 | 2HG | ARG | A | 478 | 6.482 | 37.296 | −9.063 | 1.00 | 0.00 | H |
| ATOM | 4884 | 1HD | ARG | A | 478 | 4.864 | 37.105 | −10.734 | 1.00 | 0.00 | H |
| ATOM | 4885 | 2HD | ARG | A | 478 | 4.127 | 38.284 | −9.623 | 1.00 | 0.00 | H |
| ATOM | 4886 | HE | ARG | A | 478 | 5.559 | 38.894 | −12.118 | 1.00 | 0.00 | H |
| ATOM | 4887 | 1HH1 | ARG | A | 478 | 2.562 | 41.242 | −10.702 | 1.00 | 0.00 | H |
| ATOM | 4888 | 2HH1 | ARG | A | 478 | 3.170 | 39.929 | −9.719 | 1.00 | 0.00 | H |
| ATOM | 4889 | 1HH2 | ARG | A | 478 | 3.411 | 41.620 | −12.768 | 1.00 | 0.00 | H |
| ATOM | 4890 | 2HH2 | ARG | A | 478 | 4.683 | 40.605 | −13.409 | 1.00 | 0.00 | H |
| ATOM | 4891 | N | ASP | A | 479 | 6.039 | 42.081 | −9.882 | 1.00 | 0.00 | N |
| ATOM | 4892 | CA | ASP | A | 479 | 5.794 | 43.196 | −10.798 | 1.00 | 0.00 | C |
| ATOM | 4893 | C | ASP | A | 479 | 7.025 | 44.089 | −10.821 | 1.00 | 0.00 | C |
| ATOM | 4894 | O | ASP | A | 479 | 7.519 | 44.475 | −11.876 | 1.00 | 0.00 | O |
| ATOM | 4895 | CB | ASP | A | 479 | 4.650 | 44.100 | −10.318 | 1.00 | 0.00 | C |
| ATOM | 4896 | CG | ASP | A | 479 | 3.263 | 43.517 | −10.419 | 1.00 | 0.00 | C |
| ATOM | 4897 | OD1 | ASP | A | 479 | 2.937 | 42.938 | −11.473 | 1.00 | 0.00 | O |
| ATOM | 4898 | OD2 | ASP | A | 479 | 2.558 | 43.571 | −9.390 | 1.00 | 0.00 | O1− |
| ATOM | 4899 | H | ASP | A | 479 | 5.322 | 41.875 | −9.201 | 1.00 | 0.00 | H |
| ATOM | 4900 | HA | ASP | A | 479 | 5.669 | 42.814 | −11.811 | 1.00 | 0.00 | H |
| ATOM | 4901 | 1HB | ASP | A | 479 | 4.798 | 44.347 | −9.267 | 1.00 | 0.00 | H |
| ATOM | 4902 | 2HB | ASP | A | 479 | 4.638 | 45.016 | −10.908 | 1.00 | 0.00 | H |
| ATOM | 4903 | N | ARG | A | 480 | 7.502 | 44.445 | −9.628 | 1.00 | 0.00 | N |
| ATOM | 4904 | CA | ARG | A | 480 | 8.583 | 45.398 | −9.471 | 1.00 | 0.00 | C |
| ATOM | 4905 | C | ARG | A | 480 | 9.943 | 44.694 | −9.519 | 1.00 | 0.00 | C |
| ATOM | 4906 | O | ARG | A | 480 | 10.929 | 45.220 | −9.007 | 1.00 | 0.00 | O |
| ATOM | 4907 | CB | ARG | A | 480 | 8.343 | 46.236 | −8.204 | 1.00 | 0.00 | C |
| ATOM | 4908 | CG | ARG | A | 480 | 9.062 | 47.593 | −8.275 | 1.00 | 0.00 | C |
| ATOM | 4909 | CD | ARG | A | 480 | 8.380 | 48.643 | −7.387 | 1.00 | 0.00 | C |
| ATOM | 4910 | NE | ARG | A | 480 | 8.938 | 49.981 | −7.657 | 1.00 | 0.00 | N |
| ATOM | 4911 | CZ | ARG | A | 480 | 8.286 | 51.151 | −7.622 | 1.00 | 0.00 | C |
| ATOM | 4912 | NH1 | ARG | A | 480 | 7.002 | 51.210 | −7.286 | 1.00 | 0.00 | N1+ |
| ATOM | 4913 | NH2 | ARG | A | 480 | 8.939 | 52.257 | −7.949 | 1.00 | 0.00 | N |
| ATOM | 4914 | H | ARG | A | 480 | 7.109 | 44.046 | −8.788 | 1.00 | 0.00 | H |
| ATOM | 4915 | HA | ARG | A | 480 | 8.467 | 46.202 | −10.198 | 1.00 | 0.00 | H |
| ATOM | 4916 | 1HB | ARG | A | 480 | 7.276 | 46.420 | −8.086 | 1.00 | 0.00 | H |
| ATOM | 4917 | 2HB | ARG | A | 480 | 8.717 | 45.696 | −7.335 | 1.00 | 0.00 | H |
| ATOM | 4918 | 1HG | ARG | A | 480 | 10.092 | 47.478 | −7.939 | 1.00 | 0.00 | H |
| ATOM | 4919 | 2HG | ARG | A | 480 | 9.054 | 47.957 | −9.303 | 1.00 | 0.00 | H |
| ATOM | 4920 | 1HD | ARG | A | 480 | 7.310 | 48.655 | −7.594 | 1.00 | 0.00 | H |
| ATOM | 4921 | 2HD | ARG | A | 480 | 8.544 | 48.394 | −6.338 | 1.00 | 0.00 | H |
| ATOM | 4922 | HE | ARG | A | 480 | 9.921 | 49.957 | −7.888 | 1.00 | 0.00 | H |
| ATOM | 4923 | 1HH1 | ARG | A | 480 | 6.526 | 52.100 | −7.265 | 1.00 | 0.00 | H |
| ATOM | 4924 | 2HH1 | ARG | A | 480 | 6.501 | 50.364 | −7.052 | 1.00 | 0.00 | H |
| ATOM | 4925 | 1HH2 | ARG | A | 480 | 8.465 | 53.148 | −7.929 | 1.00 | 0.00 | H |
| ATOM | 4926 | 2HH2 | ARG | A | 480 | 9.911 | 52.209 | −8.219 | 1.00 | 0.00 | H |
| ATOM | 4927 | N | GLU | A | 481 | 9.990 | 43.533 | −10.175 | 1.00 | 0.00 | N |
| ATOM | 4928 | CA | GLU | A | 481 | 11.181 | 42.975 | −10.783 | 1.00 | 0.00 | C |
| ATOM | 4929 | C | GLU | A | 481 | 10.985 | 43.161 | −12.290 | 1.00 | 0.00 | C |
| ATOM | 4930 | O | GLU | A | 481 | 11.717 | 43.901 | −12.947 | 1.00 | 0.00 | O |
| ATOM | 4931 | CB | GLU | A | 481 | 11.322 | 41.480 | −10.440 | 1.00 | 0.00 | C |
| ATOM | 4932 | CG | GLU | A | 481 | 11.422 | 41.200 | −8.936 | 1.00 | 0.00 | C |
| ATOM | 4933 | CD | GLU | A | 481 | 12.712 | 41.713 | −8.336 | 1.00 | 0.00 | C |
| ATOM | 4934 | OE1 | GLU | A | 481 | 13.656 | 42.047 | −9.079 | 1.00 | 0.00 | O |
| ATOM | 4935 | OE2 | GLU | A | 481 | 12.784 | 41.773 | −7.094 | 1.00 | 0.00 | O1− |
| ATOM | 4936 | H | GLU | A | 481 | 9.153 | 42.976 | −10.275 | 1.00 | 0.00 | H |
| ATOM | 4937 | HA | GLU | A | 481 | 12.058 | 43.521 | −10.433 | 1.00 | 0.00 | H |
| ATOM | 4938 | 1HB | GLU | A | 481 | 10.454 | 40.938 | −10.814 | 1.00 | 0.00 | H |
| ATOM | 4939 | 2HB | GLU | A | 481 | 12.225 | 41.084 | −10.904 | 1.00 | 0.00 | H |
| ATOM | 4940 | 1HG | GLU | A | 481 | 10.594 | 41.687 | −8.419 | 1.00 | 0.00 | H |
| ATOM | 4941 | 2HG | GLU | A | 481 | 11.375 | 40.125 | −8.762 | 1.00 | 0.00 | H |
| ATOM | 4942 | N | GLU | A | 482 | 9.966 | 42.489 | −12.826 | 1.00 | 0.00 | N |
| ATOM | 4943 | CA | GLU | A | 482 | 9.666 | 42.401 | −14.242 | 1.00 | 0.00 | C |
| ATOM | 4944 | C | GLU | A | 482 | 9.595 | 43.790 | −14.886 | 1.00 | 0.00 | C |
| ATOM | 4945 | O | GLU | A | 482 | 10.270 | 44.064 | −15.880 | 1.00 | 0.00 | O |
| ATOM | 4946 | CB | GLU | A | 482 | 8.346 | 41.641 | −14.397 | 1.00 | 0.00 | C |
| ATOM | 4947 | CG | GLU | A | 482 | 8.473 | 40.178 | −13.935 | 1.00 | 0.00 | C |
| ATOM | 4948 | CD | GLU | A | 482 | 7.116 | 39.560 | −13.660 | 1.00 | 0.00 | C |
| ATOM | 4949 | OE1 | GLU | A | 482 | 6.106 | 40.112 | −14.140 | 1.00 | 0.00 | O |
| ATOM | 4950 | OE2 | GLU | A | 482 | 7.068 | 38.570 | −12.903 | 1.00 | 0.00 | O1− |
| ATOM | 4951 | H | GLU | A | 482 | 9.326 | 41.984 | −12.230 | 1.00 | 0.00 | H |
| ATOM | 4952 | HA | GLU | A | 482 | 10.418 | 41.786 | −14.736 | 1.00 | 0.00 | H |
| ATOM | 4953 | 1HB | GLU | A | 482 | 7.575 | 42.124 | −13.797 | 1.00 | 0.00 | H |
| ATOM | 4954 | 2HB | GLU | A | 482 | 8.045 | 41.644 | −15.445 | 1.00 | 0.00 | H |
| ATOM | 4955 | 1HG | GLU | A | 482 | 8.967 | 39.593 | −14.711 | 1.00 | 0.00 | H |
| ATOM | 4956 | 2HG | GLU | A | 482 | 9.063 | 40.136 | −13.019 | 1.00 | 0.00 | H |
| ATOM | 4957 | N | ALA | A | 483 | 8.767 | 44.671 | −14.327 | 1.00 | 0.00 | N |
| ATOM | 4958 | CA | ALA | A | 483 | 8.459 | 45.955 | −14.931 | 1.00 | 0.00 | C |
| ATOM | 4959 | C | ALA | A | 483 | 9.678 | 46.881 | −14.910 | 1.00 | 0.00 | C |
| ATOM | 4960 | O | ALA | A | 483 | 9.719 | 47.876 | −15.635 | 1.00 | 0.00 | O |
| ATOM | 4961 | CB | ALA | A | 483 | 7.267 | 46.599 | −14.222 | 1.00 | 0.00 | C |
| ATOM | 4962 | H | ALA | A | 483 | 8.323 | 44.459 | −13.446 | 1.00 | 0.00 | H |
| ATOM | 4963 | HA | ALA | A | 483 | 8.094 | 45.802 | −15.946 | 1.00 | 0.00 | H |
| ATOM | 4964 | 1HB | ALA | A | 483 | 7.045 | 47.561 | −14.683 | 1.00 | 0.00 | H |
| ATOM | 4965 | 2HB | ALA | A | 483 | 6.397 | 45.947 | −14.307 | 1.00 | 0.00 | H |
| ATOM | 4966 | 3HB | ALA | A | 483 | 7.507 | 46.748 | −13.169 | 1.00 | 0.00 | H |
| ATOM | 4967 | N | LEU | A | 484 | 10.694 | 46.580 | −14.095 | 1.00 | 0.00 | N |
| ATOM | 4968 | CA | LEU | A | 484 | 11.932 | 47.339 | −14.148 | 1.00 | 0.00 | C |
| ATOM | 4969 | C | LEU | A | 484 | 12.574 | 47.152 | −15.523 | 1.00 | 0.00 | C |
| ATOM | 4970 | O | LEU | A | 484 | 13.263 | 48.045 | −16.014 | 1.00 | 0.00 | O |
| ATOM | 4971 | CB | LEU | A | 484 | 12.912 | 46.921 | −13.042 | 1.00 | 0.00 | C |
| ATOM | 4972 | CG | LEU | A | 484 | 12.352 | 47.018 | −11.612 | 1.00 | 0.00 | C |
| ATOM | 4973 | CD1 | LEU | A | 484 | 13.433 | 46.552 | −10.630 | 1.00 | 0.00 | C |
| ATOM | 4974 | CD2 | LEU | A | 484 | 11.932 | 48.448 | −11.249 | 1.00 | 0.00 | C |
| ATOM | 4975 | H | LEU | A | 484 | 10.605 | 45.820 | −13.436 | 1.00 | 0.00 | H |
| ATOM | 4976 | HA | LEU | A | 484 | 11.725 | 48.389 | −13.942 | 1.00 | 0.00 | H |
| ATOM | 4977 | 1HB | LEU | A | 484 | 13.209 | 45.883 | −13.192 | 1.00 | 0.00 | H |
| ATOM | 4978 | 2HB | LEU | A | 484 | 13.794 | 47.560 | −13.076 | 1.00 | 0.00 | H |
| ATOM | 4979 | HG | LEU | A | 484 | 11.466 | 46.389 | −11.525 | 1.00 | 0.00 | H |
| ATOM | 4980 | 1HD1 | LEU | A | 484 | 13.051 | 46.615 | −9.611 | 1.00 | 0.00 | H |
| ATOM | 4981 | 2HD1 | LEU | A | 484 | 13.707 | 45.521 | −10.852 | 1.00 | 0.00 | H |
| ATOM | 4982 | 3HD1 | LEU | A | 484 | 14.312 | 47.189 | −10.727 | 1.00 | 0.00 | H |
| ATOM | 4983 | 1HD2 | LEU | A | 484 | 11.543 | 48.465 | −10.231 | 1.00 | 0.00 | H |
| ATOM | 4984 | 2HD2 | LEU | A | 484 | 12.795 | 49.109 | −11.320 | 1.00 | 0.00 | H |
| ATOM | 4985 | 3HD2 | LEU | A | 484 | 11.158 | 48.786 | −11.939 | 1.00 | 0.00 | H |
| ATOM | 4986 | N | HIS | A | 485 | 12.340 | 45.998 | −16.151 | 1.00 | 0.00 | N |
| ATOM | 4987 | CA | HIS | A | 485 | 12.907 | 45.673 | −17.446 | 1.00 | 0.00 | C |
| ATOM | 4988 | C | HIS | A | 485 | 12.048 | 46.269 | −18.569 | 1.00 | 0.00 | C |
| ATOM | 4989 | O | HIS | A | 485 | 12.329 | 46.056 | −19.749 | 1.00 | 0.00 | O |
| ATOM | 4990 | CB | HIS | A | 485 | 13.086 | 44.153 | −17.585 | 1.00 | 0.00 | C |
| ATOM | 4991 | CG | HIS | A | 485 | 14.187 | 43.750 | −18.539 | 1.00 | 0.00 | C |
| ATOM | 4992 | CD2 | HIS | A | 485 | 15.061 | 42.690 | −18.539 | 1.00 | 0.00 | C |
| ATOM | 4993 | ND1 | HIS | A | 485 | 14.517 | 44.489 | −19.670 | 1.00 | 0.00 | N |
| ATOM | 4994 | CE1 | HIS | A | 485 | 15.544 | 43.881 | −20.284 | 1.00 | 0.00 | C |
| ATOM | 4995 | NE2 | HIS | A | 485 | 15.911 | 42.784 | −19.631 | 1.00 | 0.00 | N |
| ATOM | 4996 | H | HIS | A | 485 | 11.744 | 45.303 | −15.724 | 1.00 | 0.00 | H |
| ATOM | 4997 | HA | HIS | A | 485 | 13.945 | 46.004 | −17.482 | 1.00 | 0.00 | H |
| ATOM | 4998 | 1HB | HIS | A | 485 | 13.329 | 43.725 | −16.612 | 1.00 | 0.00 | H |
| ATOM | 4999 | 2HB | HIS | A | 485 | 12.161 | 43.710 | −17.955 | 1.00 | 0.00 | H |
| ATOM | 5000 | HD2 | HIS | A | 485 | 15.008 | 41.948 | −17.756 | 1.00 | 0.00 | H |
| ATOM | 5001 | HD1 | HIS | A | 485 | 14.000 | 45.327 | −19.894 | 1.00 | 0.00 | H |
| ATOM | 5002 | HE1 | HIS | A | 485 | 15.957 | 44.301 | −21.189 | 1.00 | 0.00 | H |
| ATOM | 5003 | N | GLN | A | 486 | 11.019 | 47.049 | −18.232 | 1.00 | 0.00 | N |
| ATOM | 5004 | CA | GLN | A | 486 | 10.378 | 47.916 | −19.198 | 1.00 | 0.00 | C |
| ATOM | 5005 | C | GLN | A | 486 | 11.178 | 49.221 | −19.242 | 1.00 | 0.00 | C |
| ATOM | 5006 | O | GLN | A | 486 | 12.275 | 49.263 | −19.805 | 1.00 | 0.00 | O |
| ATOM | 5007 | CB | GLN | A | 486 | 8.889 | 48.120 | −18.888 | 1.00 | 0.00 | C |
| ATOM | 5008 | CG | GLN | A | 486 | 8.072 | 46.856 | −19.152 | 1.00 | 0.00 | C |
| ATOM | 5009 | CD | GLN | A | 486 | 6.599 | 47.132 | −18.891 | 1.00 | 0.00 | C |
| ATOM | 5010 | NE2 | GLN | A | 486 | 5.984 | 47.961 | −19.725 | 1.00 | 0.00 | N |
| ATOM | 5011 | OE1 | GLN | A | 486 | 6.039 | 46.637 | −17.923 | 1.00 | 0.00 | O |
| ATOM | 5012 | H | GLN | A | 486 | 10.671 | 47.044 | −17.284 | 1.00 | 0.00 | H |
| ATOM | 5013 | HA | GLN | A | 486 | 10.296 | 47.398 | −20.154 | 1.00 | 0.00 | H |
| ATOM | 5014 | 1HB | GLN | A | 486 | 8.770 | 48.390 | −17.839 | 1.00 | 0.00 | H |
| ATOM | 5015 | 2HB | GLN | A | 486 | 8.493 | 48.919 | −19.515 | 1.00 | 0.00 | H |
| ATOM | 5016 | 1HG | GLN | A | 486 | 8.202 | 46.548 | −20.190 | 1.00 | 0.00 | H |
| ATOM | 5017 | 2HG | GLN | A | 486 | 8.412 | 46.058 | −18.492 | 1.00 | 0.00 | H |
| ATOM | 5018 | 1HE2 | GLN | A | 486 | 5.006 | 48.176 | −19.593 | 1.00 | 0.00 | H |
| ATOM | 5019 | 2HE2 | GLN | A | 486 | 6.493 | 48.377 | −20.491 | 1.00 | 0.00 | H |
| ATOM | 5020 | N | PHE | A | 487 | 10.618 | 50.280 | −18.655 | 1.00 | 0.00 | N |
| ATOM | 5021 | CA | PHE | A | 487 | 11.095 | 51.648 | −18.744 | 1.00 | 0.00 | C |
| ATOM | 5022 | C | PHE | A | 487 | 12.585 | 51.745 | −18.438 | 1.00 | 0.00 | C |
| ATOM | 5023 | O | PHE | A | 487 | 13.343 | 52.346 | −19.195 | 1.00 | 0.00 | O |
| ATOM | 5024 | CB | PHE | A | 487 | 10.274 | 52.510 | −17.783 | 1.00 | 0.00 | C |
| ATOM | 5025 | CG | PHE | A | 487 | 10.577 | 53.991 | −17.843 | 1.00 | 0.00 | C |
| ATOM | 5026 | CD1 | PHE | A | 487 | 9.947 | 54.795 | −18.810 | 1.00 | 0.00 | C |
| ATOM | 5027 | CD2 | PHE | A | 487 | 11.452 | 54.593 | −16.918 | 1.00 | 0.00 | C |
| ATOM | 5028 | CE1 | PHE | A | 487 | 10.143 | 56.183 | −18.814 | 1.00 | 0.00 | C |
| ATOM | 5029 | CE2 | PHE | A | 487 | 11.654 | 55.984 | −16.924 | 1.00 | 0.00 | C |
| ATOM | 5030 | CZ | PHE | A | 487 | 10.982 | 56.780 | −17.866 | 1.00 | 0.00 | C |
| ATOM | 5031 | H | PHE | A | 487 | 9.789 | 50.165 | −18.089 | 1.00 | 0.00 | H |
| ATOM | 5032 | HA | PHE | A | 487 | 10.879 | 52.044 | −19.737 | 1.00 | 0.00 | H |
| ATOM | 5033 | 1HB | PHE | A | 487 | 9.213 | 52.398 | −18.008 | 1.00 | 0.00 | H |
| ATOM | 5034 | 2HB | PHE | A | 487 | 10.463 | 52.193 | −16.757 | 1.00 | 0.00 | H |
| ATOM | 5035 | HD1 | PHE | A | 487 | 9.309 | 54.344 | −19.555 | 1.00 | 0.00 | H |
| ATOM | 5036 | HD2 | PHE | A | 487 | 11.976 | 53.985 | −16.195 | 1.00 | 0.00 | H |
| ATOM | 5037 | HE1 | PHE | A | 487 | 9.645 | 56.796 | −19.552 | 1.00 | 0.00 | H |
| ATOM | 5038 | HE2 | PHE | A | 487 | 12.323 | 56.436 | −16.207 | 1.00 | 0.00 | H |
| ATOM | 5039 | HZ | PHE | A | 487 | 11.112 | 57.852 | −17.858 | 1.00 | 0.00 | H |
| ATOM | 5040 | N | ARG | A | 488 | 13.026 | 51.130 | −17.341 | 1.00 | 0.00 | N |
| ATOM | 5041 | CA | ARG | A | 488 | 14.405 | 51.264 | −16.901 | 1.00 | 0.00 | C |
| ATOM | 5042 | C | ARG | A | 488 | 15.334 | 50.305 | −17.656 | 1.00 | 0.00 | C |
| ATOM | 5043 | O | ARG | A | 488 | 16.439 | 50.025 | −17.199 | 1.00 | 0.00 | O |
| ATOM | 5044 | CB | ARG | A | 488 | 14.514 | 51.137 | −15.374 | 1.00 | 0.00 | C |
| ATOM | 5045 | CG | ARG | A | 488 | 13.686 | 52.209 | −14.646 | 1.00 | 0.00 | C |
| ATOM | 5046 | CD | ARG | A | 488 | 14.056 | 52.225 | −13.158 | 1.00 | 0.00 | C |
| ATOM | 5047 | NE | ARG | A | 488 | 13.337 | 53.266 | −12.392 | 1.00 | 0.00 | N |
| ATOM | 5048 | CZ | ARG | A | 488 | 12.541 | 53.021 | −11.338 | 1.00 | 0.00 | C |
| ATOM | 5049 | NH1 | ARG | A | 488 | 11.914 | 51.850 | −11.244 | 1.00 | 0.00 | N1+ |
| ATOM | 5050 | NH2 | ARG | A | 488 | 12.424 | 53.929 | −10.375 | 1.00 | 0.00 | N |
| ATOM | 5051 | H | ARG | A | 488 | 12.398 | 50.555 | −16.797 | 1.00 | 0.00 | H |
| ATOM | 5052 | HA | ARG | A | 488 | 14.704 | 52.311 | −16.955 | 1.00 | 0.00 | H |
| ATOM | 5053 | 1HB | ARG | A | 488 | 14.150 | 50.157 | −15.064 | 1.00 | 0.00 | H |
| ATOM | 5054 | 2HB | ARG | A | 488 | 15.556 | 51.249 | −15.073 | 1.00 | 0.00 | H |
| ATOM | 5055 | 1HG | ARG | A | 488 | 13.894 | 53.187 | −15.080 | 1.00 | 0.00 | H |
| ATOM | 5056 | 2HG | ARG | A | 488 | 12.625 | 51.984 | −14.751 | 1.00 | 0.00 | H |
| ATOM | 5057 | 1HD | ARG | A | 488 | 13.812 | 51.261 | −12.712 | 1.00 | 0.00 | H |
| ATOM | 5058 | 2HD | ARG | A | 488 | 15.124 | 52.416 | −13.051 | 1.00 | 0.00 | H |
| ATOM | 5059 | HE | ARG | A | 488 | 13.476 | 54.215 | −12.710 | 1.00 | 0.00 | H |
| ATOM | 5060 | 1HH1 | ARG | A | 488 | 11.314 | 51.661 | −10.454 | 1.00 | 0.00 | H |
| ATOM | 5061 | 2HH1 | ARG | A | 488 | 12.037 | 51.152 | −11.963 | 1.00 | 0.00 | H |
| ATOM | 5062 | 1HH2 | ARG | A | 488 | 11.826 | 53.748 | −9.582 | 1.00 | 0.00 | H |
| ATOM | 5063 | 2HH2 | ARG | A | 488 | 12.932 | 54.799 | −10.437 | 1.00 | 0.00 | H |
| ATOM | 5064 | N | SER | A | 489 | 14.938 | 49.844 | −18.844 | 1.00 | 0.00 | N |
| ATOM | 5065 | CA | SER | A | 489 | 15.883 | 49.359 | −19.831 | 1.00 | 0.00 | C |
| ATOM | 5066 | C | SER | A | 489 | 15.525 | 49.928 | −21.209 | 1.00 | 0.00 | C |
| ATOM | 5067 | O | SER | A | 489 | 15.951 | 49.383 | −22.228 | 1.00 | 0.00 | O |
| ATOM | 5068 | CB | SER | A | 489 | 15.860 | 47.826 | −19.836 | 1.00 | 0.00 | C |
| ATOM | 5069 | OG | SER | A | 489 | 14.617 | 47.345 | −20.327 | 1.00 | 0.00 | O |
| ATOM | 5070 | H | SER | A | 489 | 13.955 | 49.826 | −19.077 | 1.00 | 0.00 | H |
| ATOM | 5071 | HA | SER | A | 489 | 16.894 | 49.644 | −19.539 | 1.00 | 0.00 | H |
| ATOM | 5072 | 1HB | SER | A | 489 | 16.659 | 47.452 | −20.476 | 1.00 | 0.00 | H |
| ATOM | 5073 | 2HB | SER | A | 489 | 16.004 | 47.457 | −18.821 | 1.00 | 0.00 | H |
| ATOM | 5074 | HG | SER | A | 489 | 14.621 | 46.385 | −20.323 | 1.00 | 0.00 | H |
| ATOM | 5075 | N | GLY | A | 490 | 14.677 | 50.959 | −21.246 | 1.00 | 0.00 | N |
| ATOM | 5076 | CA | GLY | A | 490 | 14.101 | 51.519 | −22.456 | 1.00 | 0.00 | C |
| ATOM | 5077 | C | GLY | A | 490 | 13.215 | 50.486 | −23.156 | 1.00 | 0.00 | C |
| ATOM | 5078 | O | GLY | A | 490 | 13.717 | 49.620 | −23.873 | 1.00 | 0.00 | O |
| ATOM | 5079 | H | GLY | A | 490 | 14.391 | 51.409 | −20.388 | 1.00 | 0.00 | H |
| ATOM | 5080 | 1HA | GLY | A | 490 | 13.497 | 52.390 | −22.202 | 1.00 | 0.00 | H |
| ATOM | 5081 | 2HA | GLY | A | 490 | 14.900 | 51.818 | −23.134 | 1.00 | 0.00 | H |
| ATOM | 5082 | N | LYS | A | 491 | 11.899 | 50.570 | −22.963 | 1.00 | 0.00 | N |
| ATOM | 5083 | CA | LYS | A | 491 | 10.905 | 49.807 | −23.708 | 1.00 | 0.00 | C |
| ATOM | 5084 | C | LYS | A | 491 | 9.733 | 50.767 | −23.884 | 1.00 | 0.00 | C |
| ATOM | 5085 | O | LYS | A | 491 | 9.784 | 51.618 | −24.766 | 1.00 | 0.00 | O |
| ATOM | 5086 | CB | LYS | A | 491 | 10.517 | 48.494 | −22.993 | 1.00 | 0.00 | C |
| ATOM | 5087 | CG | LYS | A | 491 | 11.677 | 47.523 | −22.722 | 1.00 | 0.00 | C |
| ATOM | 5088 | CD | LYS | A | 491 | 12.290 | 46.927 | −24.001 | 1.00 | 0.00 | C |
| ATOM | 5089 | CE | LYS | A | 491 | 13.647 | 46.264 | −23.723 | 1.00 | 0.00 | C |
| ATOM | 5090 | NZ | LYS | A | 491 | 14.601 | 47.203 | −23.099 | 1.00 | 0.00 | N1+ |
| ATOM | 5091 | H | LYS | A | 491 | 11.530 | 51.194 | −22.260 | 1.00 | 0.00 | H |
| ATOM | 5092 | HA | LYS | A | 491 | 11.343 | 49.452 | −24.641 | 1.00 | 0.00 | H |
| ATOM | 5093 | 1HB | LYS | A | 491 | 10.076 | 48.725 | −22.023 | 1.00 | 0.00 | H |
| ATOM | 5094 | 2HB | LYS | A | 491 | 9.794 | 47.949 | −23.599 | 1.00 | 0.00 | H |
| ATOM | 5095 | 1HG | LYS | A | 491 | 12.473 | 48.045 | −22.192 | 1.00 | 0.00 | H |
| ATOM | 5096 | 2HG | LYS | A | 491 | 11.320 | 46.691 | −22.114 | 1.00 | 0.00 | H |
| ATOM | 5097 | 1HD | LYS | A | 491 | 11.617 | 46.174 | −24.411 | 1.00 | 0.00 | H |
| ATOM | 5098 | 2HD | LYS | A | 491 | 12.439 | 47.719 | −24.735 | 1.00 | 0.00 | H |
| ATOM | 5099 | 1HE | LYS | A | 491 | 13.507 | 45.420 | −23.047 | 1.00 | 0.00 | H |
| ATOM | 5100 | 2HE | LYS | A | 491 | 14.078 | 45.911 | −24.660 | 1.00 | 0.00 | H |
| ATOM | 5101 | 1HZ | LYS | A | 491 | 15.479 | 46.732 | −22.932 | 1.00 | 0.00 | H |
| ATOM | 5102 | 2HZ | LYS | A | 491 | 14.754 | 47.988 | −23.717 | 1.00 | 0.00 | H |
| ATOM | 5103 | 3HZ | LYS | A | 491 | 14.225 | 47.534 | −22.222 | 1.00 | 0.00 | H |
| ATOM | 5104 | N | SER | A | 492 | 8.742 | 50.710 | −22.996 | 1.00 | 0.00 | N |
| ATOM | 5105 | CA | SER | A | 492 | 7.901 | 51.852 | −22.700 | 1.00 | 0.00 | C |
| ATOM | 5106 | C | SER | A | 492 | 8.810 | 53.078 | −22.490 | 1.00 | 0.00 | C |
| ATOM | 5107 | O | SER | A | 492 | 9.715 | 52.993 | −21.658 | 1.00 | 0.00 | O |
| ATOM | 5108 | CB | SER | A | 492 | 7.163 | 51.525 | −21.390 | 1.00 | 0.00 | C |
| ATOM | 5109 | OG | SER | A | 492 | 6.715 | 50.171 | −21.362 | 1.00 | 0.00 | O |
| ATOM | 5110 | H | SER | A | 492 | 8.555 | 49.849 | −22.501 | 1.00 | 0.00 | H |
| ATOM | 5111 | HA | SER | A | 492 | 7.247 | 52.052 | −23.548 | 1.00 | 0.00 | H |
| ATOM | 5112 | 1HB | SER | A | 492 | 7.834 | 51.682 | −20.545 | 1.00 | 0.00 | H |
| ATOM | 5113 | 2HB | SER | A | 492 | 6.294 | 52.175 | −21.290 | 1.00 | 0.00 | H |
| ATOM | 5114 | HG | SER | A | 492 | 6.260 | 50.000 | −20.534 | 1.00 | 0.00 | H |
| ATOM | 5115 | N | PRO | A | 493 | 8.609 | 54.206 | −23.184 | 1.00 | 0.00 | N |
| ATOM | 5116 | CA | PRO | A | 493 | 9.433 | 55.396 | −23.002 | 1.00 | 0.00 | C |
| ATOM | 5117 | C | PRO | A | 493 | 8.709 | 56.375 | −22.066 | 1.00 | 0.00 | C |
| ATOM | 5118 | O | PRO | A | 493 | 9.104 | 57.533 | −21.920 | 1.00 | 0.00 | O |
| ATOM | 5119 | CB | PRO | A | 493 | 9.598 | 55.962 | −24.412 | 1.00 | 0.00 | C |
| ATOM | 5120 | CG | PRO | A | 493 | 8.254 | 55.636 | −25.067 | 1.00 | 0.00 | C |
| ATOM | 5121 | CD | PRO | A | 493 | 7.852 | 54.304 | −24.427 | 1.00 | 0.00 | C |
| ATOM | 5122 | HA | PRO | A | 493 | 10.391 | 55.112 | −22.566 | 1.00 | 0.00 | H |
| ATOM | 5123 | 1HB | PRO | A | 493 | 9.788 | 57.034 | −24.354 | 1.00 | 0.00 | H |
| ATOM | 5124 | 2HB | PRO | A | 493 | 10.436 | 55.472 | −24.906 | 1.00 | 0.00 | H |
| ATOM | 5125 | 1HG | PRO | A | 493 | 7.540 | 56.430 | −24.849 | 1.00 | 0.00 | H |
| ATOM | 5126 | 2HG | PRO | A | 493 | 8.385 | 55.552 | −26.146 | 1.00 | 0.00 | H |
| ATOM | 5127 | 1HD | PRO | A | 493 | 8.107 | 53.465 | −25.074 | 1.00 | 0.00 | H |
| ATOM | 5128 | 2HD | PRO | A | 493 | 6.785 | 54.281 | −24.206 | 1.00 | 0.00 | H |
| ATOM | 5129 | N | ILE | A | 494 | 7.650 | 55.869 | −21.433 | 1.00 | 0.00 | N |
| ATOM | 5130 | CA | ILE | A | 494 | 6.713 | 56.548 | −20.560 | 1.00 | 0.00 | C |
| ATOM | 5131 | C | ILE | A | 494 | 6.493 | 55.599 | −19.376 | 1.00 | 0.00 | C |
| ATOM | 5132 | O | ILE | A | 494 | 6.607 | 54.378 | −19.522 | 1.00 | 0.00 | O |
| ATOM | 5133 | CB | ILE | A | 494 | 5.402 | 56.825 | −21.330 | 1.00 | 0.00 | C |
| ATOM | 5134 | CG1 | ILE | A | 494 | 5.639 | 57.841 | −22.466 | 1.00 | 0.00 | C |
| ATOM | 5135 | CG2 | ILE | A | 494 | 4.309 | 57.353 | −20.387 | 1.00 | 0.00 | C |
| ATOM | 5136 | CD1 | ILE | A | 494 | 4.507 | 57.863 | −23.498 | 1.00 | 0.00 | C |
| ATOM | 5137 | H | ILE | A | 494 | 7.420 | 54.891 | −21.536 | 1.00 | 0.00 | H |
| ATOM | 5138 | HA | ILE | A | 494 | 7.134 | 57.502 | −20.245 | 1.00 | 0.00 | H |
| ATOM | 5139 | HB | ILE | A | 494 | 5.010 | 55.891 | −21.730 | 1.00 | 0.00 | H |
| ATOM | 5140 | 1HG1 | ILE | A | 494 | 5.722 | 58.843 | −22.047 | 1.00 | 0.00 | H |
| ATOM | 5141 | 2HG1 | ILE | A | 494 | 6.560 | 57.589 | −22.993 | 1.00 | 0.00 | H |
| ATOM | 5142 | 1HG2 | ILE | A | 494 | 3.398 | 57.540 | −20.956 | 1.00 | 0.00 | H |
| ATOM | 5143 | 2HG2 | ILE | A | 494 | 4.108 | 56.613 | −19.613 | 1.00 | 0.00 | H |
| ATOM | 5144 | 3HG2 | ILE | A | 494 | 4.645 | 58.281 | −19.924 | 1.00 | 0.00 | H |
| ATOM | 5145 | 1HD1 | ILE | A | 494 | 4.734 | 58.598 | −24.271 | 1.00 | 0.00 | H |
| ATOM | 5146 | 2HD1 | ILE | A | 494 | 4.409 | 56.877 | −23.953 | 1.00 | 0.00 | H |
| ATOM | 5147 | 3HD1 | ILE | A | 494 | 3.572 | 58.131 | −23.007 | 1.00 | 0.00 | H |
| ATOM | 5148 | N | LEU | A | 495 | 6.205 | 56.153 | −18.202 | 1.00 | 0.00 | N |
| ATOM | 5149 | CA | LEU | A | 495 | 6.095 | 55.440 | −16.942 | 1.00 | 0.00 | C |
| ATOM | 5150 | C | LEU | A | 495 | 5.222 | 56.300 | −16.032 | 1.00 | 0.00 | C |
| ATOM | 5151 | O | LEU | A | 495 | 5.254 | 57.525 | −16.142 | 1.00 | 0.00 | O |
| ATOM | 5152 | CB | LEU | A | 495 | 7.511 | 55.253 | −16.364 | 1.00 | 0.00 | C |
| ATOM | 5153 | CG | LEU | A | 495 | 7.605 | 54.813 | −14.891 | 1.00 | 0.00 | C |
| ATOM | 5154 | CD1 | LEU | A | 495 | 7.022 | 53.419 | −14.662 | 1.00 | 0.00 | C |
| ATOM | 5155 | CD2 | LEU | A | 495 | 9.072 | 54.768 | −14.445 | 1.00 | 0.00 | C |
| ATOM | 5156 | H | LEU | A | 495 | 6.042 | 57.148 | −18.140 | 1.00 | 0.00 | H |
| ATOM | 5157 | HA | LEU | A | 495 | 5.642 | 54.463 | −17.115 | 1.00 | 0.00 | H |
| ATOM | 5158 | 1HB | LEU | A | 495 | 8.039 | 54.488 | −16.934 | 1.00 | 0.00 | H |
| ATOM | 5159 | 2HB | LEU | A | 495 | 8.056 | 56.194 | −16.427 | 1.00 | 0.00 | H |
| ATOM | 5160 | HG | LEU | A | 495 | 7.043 | 55.509 | −14.267 | 1.00 | 0.00 | H |
| ATOM | 5161 | 1HD1 | LEU | A | 495 | 7.112 | 53.156 | −13.608 | 1.00 | 0.00 | H |
| ATOM | 5162 | 2HD1 | LEU | A | 495 | 5.971 | 53.412 | −14.949 | 1.00 | 0.00 | H |
| ATOM | 5163 | 3HD1 | LEU | A | 495 | 7.568 | 52.693 | −15.265 | 1.00 | 0.00 | H |
| ATOM | 5164 | 1HD2 | LEU | A | 495 | 9.126 | 54.456 | −13.402 | 1.00 | 0.00 | H |
| ATOM | 5165 | 2HD2 | LEU | A | 495 | 9.619 | 54.057 | −15.065 | 1.00 | 0.00 | H |
| ATOM | 5166 | 3HD2 | LEU | A | 495 | 9.515 | 55.758 | −14.551 | 1.00 | 0.00 | H |
| ATOM | 5167 | N | VAL | A | 496 | 4.473 | 55.669 | −15.131 | 1.00 | 0.00 | N |
| ATOM | 5168 | CA | VAL | A | 496 | 3.740 | 56.328 | −14.062 | 1.00 | 0.00 | C |
| ATOM | 5169 | C | VAL | A | 496 | 4.296 | 55.760 | −12.748 | 1.00 | 0.00 | C |
| ATOM | 5170 | O | VAL | A | 496 | 4.567 | 54.557 | −12.672 | 1.00 | 0.00 | O |
| ATOM | 5171 | CB | VAL | A | 496 | 2.230 | 56.061 | −14.227 | 1.00 | 0.00 | C |
| ATOM | 5172 | CG1 | VAL | A | 496 | 1.408 | 56.878 | −13.225 | 1.00 | 0.00 | C |
| ATOM | 5173 | CG2 | VAL | A | 496 | 1.727 | 56.326 | −15.655 | 1.00 | 0.00 | C |
| ATOM | 5174 | H | VAL | A | 496 | 4.386 | 54.663 | −15.162 | 1.00 | 0.00 | H |
| ATOM | 5175 | HA | VAL | A | 496 | 3.874 | 57.407 | −14.140 | 1.00 | 0.00 | H |
| ATOM | 5176 | HB | VAL | A | 496 | 2.037 | 54.992 | −14.133 | 1.00 | 0.00 | H |
| ATOM | 5177 | 1HG1 | VAL | A | 496 | 0.348 | 56.668 | −13.367 | 1.00 | 0.00 | H |
| ATOM | 5178 | 2HG1 | VAL | A | 496 | 1.698 | 56.608 | −12.210 | 1.00 | 0.00 | H |
| ATOM | 5179 | 3HG1 | VAL | A | 496 | 1.591 | 57.941 | −13.385 | 1.00 | 0.00 | H |
| ATOM | 5180 | 1HG2 | VAL | A | 496 | 0.658 | 56.121 | −15.709 | 1.00 | 0.00 | H |
| ATOM | 5181 | 2HG2 | VAL | A | 496 | 1.910 | 57.369 | −15.917 | 1.00 | 0.00 | H |
| ATOM | 5182 | 3HG2 | VAL | A | 496 | 2.257 | 55.679 | −16.354 | 1.00 | 0.00 | H |
| ATOM | 5183 | N | ALA | A | 497 | 4.501 | 56.585 | −11.718 | 1.00 | 0.00 | N |
| ATOM | 5184 | CA | ALA | A | 497 | 5.113 | 56.142 | −10.471 | 1.00 | 0.00 | C |
| ATOM | 5185 | C | ALA | A | 497 | 4.557 | 56.913 | −9.275 | 1.00 | 0.00 | C |
| ATOM | 5186 | O | ALA | A | 497 | 4.336 | 58.119 | −9.358 | 1.00 | 0.00 | O |
| ATOM | 5187 | CB | ALA | A | 497 | 6.630 | 56.326 | −10.544 | 1.00 | 0.00 | C |
| ATOM | 5188 | H | ALA | A | 497 | 4.230 | 57.555 | −11.787 | 1.00 | 0.00 | H |
| ATOM | 5189 | HA | ALA | A | 497 | 4.937 | 55.074 | −10.338 | 1.00 | 0.00 | H |
| ATOM | 5190 | 1HB | ALA | A | 497 | 7.083 | 55.994 | −9.610 | 1.00 | 0.00 | H |
| ATOM | 5191 | 2HB | ALA | A | 497 | 7.029 | 55.737 | −11.371 | 1.00 | 0.00 | H |
| ATOM | 5192 | 3HB | ALA | A | 497 | 6.862 | 57.379 | −10.705 | 1.00 | 0.00 | H |
| ATOM | 5193 | N | THR | A | 498 | 4.358 | 56.207 | −8.163 | 1.00 | 0.00 | N |
| ATOM | 5194 | CA | THR | A | 498 | 3.839 | 56.762 | −6.927 | 1.00 | 0.00 | C |
| ATOM | 5195 | C | THR | A | 498 | 5.010 | 57.258 | −6.078 | 1.00 | 0.00 | C |
| ATOM | 5196 | O | THR | A | 498 | 5.941 | 56.480 | −5.849 | 1.00 | 0.00 | O |
| ATOM | 5197 | CB | THR | A | 498 | 3.076 | 55.646 | −6.205 | 1.00 | 0.00 | C |
| ATOM | 5198 | CG2 | THR | A | 498 | 1.724 | 55.377 | −6.872 | 1.00 | 0.00 | C |
| ATOM | 5199 | OG1 | THR | A | 498 | 3.875 | 54.474 | −6.202 | 1.00 | 0.00 | O |
| ATOM | 5200 | H | THR | A | 498 | 4.573 | 55.221 | −8.145 | 1.00 | 0.00 | H |
| ATOM | 5201 | HA | THR | A | 498 | 3.169 | 57.592 | −7.154 | 1.00 | 0.00 | H |
| ATOM | 5202 | HB | THR | A | 498 | 2.934 | 55.919 | −5.160 | 1.00 | 0.00 | H |
| ATOM | 5203 | 1HG2 | THR | A | 498 | 1.206 | 54.581 | −6.338 | 1.00 | 0.00 | H |
| ATOM | 5204 | 2HG2 | THR | A | 498 | 1.119 | 56.284 | −6.847 | 1.00 | 0.00 | H |
| ATOM | 5205 | 3HG2 | THR | A | 498 | 1.883 | 55.076 | −7.908 | 1.00 | 0.00 | H |
| ATOM | 5206 | HG1 | THR | A | 498 | 3.405 | 53.768 | −5.752 | 1.00 | 0.00 | H |
| ATOM | 5207 | N | ALA | A | 499 | 4.954 | 58.508 | −5.617 | 1.00 | 0.00 | N |
| ATOM | 5208 | CA | ALA | A | 499 | 5.856 | 59.125 | −4.655 | 1.00 | 0.00 | C |
| ATOM | 5209 | C | ALA | A | 499 | 6.407 | 58.140 | −3.618 | 1.00 | 0.00 | C |
| ATOM | 5210 | O | ALA | A | 499 | 7.618 | 58.097 | −3.370 | 1.00 | 0.00 | O |
| ATOM | 5211 | CB | ALA | A | 499 | 5.132 | 60.281 | −3.963 | 1.00 | 0.00 | C |
| ATOM | 5212 | H | ALA | A | 499 | 4.227 | 59.132 | −5.938 | 1.00 | 0.00 | H |
| ATOM | 5213 | HA | ALA | A | 499 | 6.676 | 59.611 | −5.185 | 1.00 | 0.00 | H |
| ATOM | 5214 | 1HB | ALA | A | 499 | 5.801 | 60.749 | −3.241 | 1.00 | 0.00 | H |
| ATOM | 5215 | 2HB | ALA | A | 499 | 4.828 | 61.017 | −4.707 | 1.00 | 0.00 | H |
| ATOM | 5216 | 3HB | ALA | A | 499 | 4.250 | 59.900 | −3.447 | 1.00 | 0.00 | H |
| ATOM | 5217 | N | VAL | A | 500 | 5.525 | 57.358 | −2.993 | 1.00 | 0.00 | N |
| ATOM | 5218 | CA | VAL | A | 500 | 5.854 | 56.450 | −1.904 | 1.00 | 0.00 | C |
| ATOM | 5219 | C | VAL | A | 500 | 6.912 | 55.425 | −2.335 | 1.00 | 0.00 | C |
| ATOM | 5220 | O | VAL | A | 500 | 7.544 | 54.779 | −1.503 | 1.00 | 0.00 | O |
| ATOM | 5221 | CB | VAL | A | 500 | 4.574 | 55.792 | −1.354 | 1.00 | 0.00 | C |
| ATOM | 5222 | CG1 | VAL | A | 500 | 4.048 | 54.683 | −2.274 | 1.00 | 0.00 | C |
| ATOM | 5223 | CG2 | VAL | A | 500 | 4.787 | 55.227 | 0.057 | 1.00 | 0.00 | C |
| ATOM | 5224 | H | VAL | A | 500 | 4.553 | 57.371 | −3.270 | 1.00 | 0.00 | H |
| ATOM | 5225 | HA | VAL | A | 500 | 6.147 | 57.027 | −1.027 | 1.00 | 0.00 | H |
| ATOM | 5226 | HB | VAL | A | 500 | 3.812 | 56.555 | −1.194 | 1.00 | 0.00 | H |
| ATOM | 5227 | 1HG1 | VAL | A | 500 | 3.145 | 54.250 | −1.842 | 1.00 | 0.00 | H |
| ATOM | 5228 | 2HG1 | VAL | A | 500 | 3.817 | 55.102 | −3.253 | 1.00 | 0.00 | H |
| ATOM | 5229 | 3HG1 | VAL | A | 500 | 4.806 | 53.908 | −2.380 | 1.00 | 0.00 | H |
| ATOM | 5230 | 1HG2 | VAL | A | 500 | 3.862 | 54.771 | 0.410 | 1.00 | 0.00 | H |
| ATOM | 5231 | 2HG2 | VAL | A | 500 | 5.576 | 54.476 | 0.033 | 1.00 | 0.00 | H |
| ATOM | 5232 | 3HG2 | VAL | A | 500 | 5.075 | 56.034 | 0.732 | 1.00 | 0.00 | H |
| ATOM | 5233 | N | ALA | A | 501 | 7.106 | 55.258 | −3.642 | 1.00 | 0.00 | N |
| ATOM | 5234 | CA | ALA | A | 501 | 8.159 | 54.451 | −4.208 | 1.00 | 0.00 | C |
| ATOM | 5235 | C | ALA | A | 501 | 8.665 | 55.141 | −5.472 | 1.00 | 0.00 | C |
| ATOM | 5236 | O | ALA | A | 501 | 8.765 | 54.521 | −6.533 | 1.00 | 0.00 | O |
| ATOM | 5237 | CB | ALA | A | 501 | 7.632 | 53.045 | −4.486 | 1.00 | 0.00 | C |
| ATOM | 5238 | H | ALA | A | 501 | 6.494 | 55.712 | −4.306 | 1.00 | 0.00 | H |
| ATOM | 5239 | HA | ALA | A | 501 | 8.948 | 54.312 | −3.469 | 1.00 | 0.00 | H |
| ATOM | 5240 | 1HB | ALA | A | 501 | 8.428 | 52.436 | −4.914 | 1.00 | 0.00 | H |
| ATOM | 5241 | 2HB | ALA | A | 501 | 7.290 | 52.594 | −3.554 | 1.00 | 0.00 | H |
| ATOM | 5242 | 3HB | ALA | A | 501 | 6.800 | 53.100 | −5.189 | 1.00 | 0.00 | H |
| ATOM | 5243 | N | ALA | A | 502 | 9.007 | 56.422 | −5.342 | 1.00 | 0.00 | N |
| ATOM | 5244 | CA | ALA | A | 502 | 9.738 | 57.177 | −6.347 | 1.00 | 0.00 | C |
| ATOM | 5245 | C | ALA | A | 502 | 10.956 | 57.853 | −5.708 | 1.00 | 0.00 | C |
| ATOM | 5246 | O | ALA | A | 502 | 11.690 | 58.578 | −6.377 | 1.00 | 0.00 | O |
| ATOM | 5247 | CB | ALA | A | 502 | 8.815 | 58.195 | −7.007 | 1.00 | 0.00 | C |
| ATOM | 5248 | H | ALA | A | 502 | 8.757 | 56.929 | −4.505 | 1.00 | 0.00 | H |
| ATOM | 5249 | HA | ALA | A | 502 | 10.024 | 56.514 | −7.164 | 1.00 | 0.00 | H |
| ATOM | 5250 | 1HB | ALA | A | 502 | 9.370 | 58.757 | −7.759 | 1.00 | 0.00 | H |
| ATOM | 5251 | 2HB | ALA | A | 502 | 7.983 | 57.677 | −7.484 | 1.00 | 0.00 | H |
| ATOM | 5252 | 3HB | ALA | A | 502 | 8.430 | 58.881 | −6.253 | 1.00 | 0.00 | H |
| ATOM | 5253 | N | ARG | A | 503 | 11.181 | 57.585 | −4.421 | 1.00 | 0.00 | N |
| ATOM | 5254 | CA | ARG | A | 503 | 12.491 | 57.676 | −3.801 | 1.00 | 0.00 | C |
| ATOM | 5255 | C | ARG | A | 503 | 13.014 | 56.249 | −3.966 | 1.00 | 0.00 | C |
| ATOM | 5256 | O | ARG | A | 503 | 12.482 | 55.326 | −3.350 | 1.00 | 0.00 | O |
| ATOM | 5257 | CB | ARG | A | 503 | 12.353 | 58.124 | −2.329 | 1.00 | 0.00 | C |
| ATOM | 5258 | CG | ARG | A | 503 | 11.600 | 59.464 | −2.260 | 1.00 | 0.00 | C |
| ATOM | 5259 | CD | ARG | A | 503 | 11.704 | 60.241 | −0.937 | 1.00 | 0.00 | C |
| ATOM | 5260 | NE | ARG | A | 503 | 10.518 | 61.107 | −0.721 | 1.00 | 0.00 | N |
| ATOM | 5261 | CZ | ARG | A | 503 | 10.081 | 61.576 | 0.465 | 1.00 | 0.00 | C |
| ATOM | 5262 | NH1 | ARG | A | 503 | 10.816 | 61.416 | 1.555 | 1.00 | 0.00 | N1+ |
| ATOM | 5263 | NH2 | ARG | A | 503 | 8.889 | 62.158 | 0.573 | 1.00 | 0.00 | N |
| ATOM | 5264 | H | ARG | A | 503 | 10.417 | 57.303 | −3.823 | 1.00 | 0.00 | H |
| ATOM | 5265 | HA | ARG | A | 503 | 13.086 | 58.431 | −4.315 | 1.00 | 0.00 | H |
| ATOM | 5266 | 1HB | ARG | A | 503 | 11.799 | 57.370 | −1.769 | 1.00 | 0.00 | H |
| ATOM | 5267 | 2HB | ARG | A | 503 | 13.344 | 58.245 | −1.891 | 1.00 | 0.00 | H |
| ATOM | 5268 | 1HG | ARG | A | 503 | 11.980 | 60.135 | −3.030 | 1.00 | 0.00 | H |
| ATOM | 5269 | 2HG | ARG | A | 503 | 10.536 | 59.292 | −2.421 | 1.00 | 0.00 | H |
| ATOM | 5270 | 1HD | ARG | A | 503 | 11.773 | 59.538 | −0.107 | 1.00 | 0.00 | H |
| ATOM | 5271 | 2HD | ARG | A | 503 | 12.593 | 60.871 | −0.955 | 1.00 | 0.00 | H |
| ATOM | 5272 | HE | ARG | A | 503 | 10.024 | 61.342 | −1.570 | 1.00 | 0.00 | H |
| ATOM | 5273 | 1HH1 | ARG | A | 503 | 10.486 | 61.768 | 2.442 | 1.00 | 0.00 | H |
| ATOM | 5274 | 2HH1 | ARG | A | 503 | 11.706 | 60.943 | 1.499 | 1.00 | 0.00 | H |
| ATOM | 5275 | 1HH2 | ARG | A | 503 | 8.576 | 62.504 | 1.469 | 1.00 | 0.00 | H |
| ATOM | 5276 | 2HH2 | ARG | A | 503 | 8.298 | 62.255 | −0.240 | 1.00 | 0.00 | H |
| ATOM | 5277 | N | GLY | A | 504 | 13.930 | 56.017 | −4.906 | 1.00 | 0.00 | N |
| ATOM | 5278 | CA | GLY | A | 504 | 14.085 | 54.688 | −5.471 | 1.00 | 0.00 | C |
| ATOM | 5279 | C | GLY | A | 504 | 15.131 | 54.655 | −6.582 | 1.00 | 0.00 | C |
| ATOM | 5280 | O | GLY | A | 504 | 16.243 | 55.148 | −6.406 | 1.00 | 0.00 | O |
| ATOM | 5281 | H | GLY | A | 504 | 14.523 | 56.768 | −5.231 | 1.00 | 0.00 | H |
| ATOM | 5282 | 1HA | GLY | A | 504 | 14.397 | 53.995 | −4.690 | 1.00 | 0.00 | H |
| ATOM | 5283 | 2HA | GLY | A | 504 | 13.134 | 54.357 | −5.890 | 1.00 | 0.00 | H |
| ATOM | 5284 | N | LEU | A | 505 | 14.798 | 54.060 | −7.728 | 1.00 | 0.00 | N |
| ATOM | 5285 | CA | LEU | A | 505 | 15.719 | 53.986 | −8.851 | 1.00 | 0.00 | C |
| ATOM | 5286 | C | LEU | A | 505 | 15.597 | 55.300 | −9.611 | 1.00 | 0.00 | C |
| ATOM | 5287 | O | LEU | A | 505 | 14.571 | 55.548 | −10.254 | 1.00 | 0.00 | O |
| ATOM | 5288 | CB | LEU | A | 505 | 15.414 | 52.777 | −9.757 | 1.00 | 0.00 | C |
| ATOM | 5289 | CG | LEU | A | 505 | 16.459 | 51.657 | −9.655 | 1.00 | 0.00 | C |
| ATOM | 5290 | CD1 | LEU | A | 505 | 15.844 | 50.306 | −10.042 | 1.00 | 0.00 | C |
| ATOM | 5291 | CD2 | LEU | A | 505 | 17.631 | 51.957 | −10.598 | 1.00 | 0.00 | C |
| ATOM | 5292 | H | LEU | A | 505 | 13.884 | 53.644 | −7.835 | 1.00 | 0.00 | H |
| ATOM | 5293 | HA | LEU | A | 505 | 16.729 | 53.809 | −8.481 | 1.00 | 0.00 | H |
| ATOM | 5294 | 1HB | LEU | A | 505 | 14.450 | 52.350 | −9.482 | 1.00 | 0.00 | H |
| ATOM | 5295 | 2HB | LEU | A | 505 | 15.384 | 53.101 | −10.797 | 1.00 | 0.00 | H |
| ATOM | 5296 | HG | LEU | A | 505 | 16.829 | 51.596 | −8.631 | 1.00 | 0.00 | H |
| ATOM | 5297 | 1HD1 | LEU | A | 505 | 16.601 | 49.526 | −9.963 | 1.00 | 0.00 | H |
| ATOM | 5298 | 2HD1 | LEU | A | 505 | 15.016 | 50.078 | −9.371 | 1.00 | 0.00 | H |
| ATOM | 5299 | 3HD1 | LEU | A | 505 | 15.478 | 50.354 | −11.067 | 1.00 | 0.00 | H |
| ATOM | 5300 | 1HD2 | LEU | A | 505 | 18.371 | 51.160 | −10.524 | 1.00 | 0.00 | H |
| ATOM | 5301 | 2HD2 | LEU | A | 505 | 17.266 | 52.018 | −11.623 | 1.00 | 0.00 | H |
| ATOM | 5302 | 3HD2 | LEU | A | 505 | 18.089 | 52.905 | −10.318 | 1.00 | 0.00 | H |
| ATOM | 5303 | N | ASP | A | 506 | 16.649 | 56.110 | −9.536 | 1.00 | 0.00 | N |
| ATOM | 5304 | CA | ASP | A | 506 | 16.773 | 57.356 | −10.267 | 1.00 | 0.00 | C |
| ATOM | 5305 | C | ASP | A | 506 | 16.659 | 57.073 | −11.768 | 1.00 | 0.00 | C |
| ATOM | 5306 | O | ASP | A | 506 | 16.782 | 55.936 | −12.229 | 1.00 | 0.00 | O |
| ATOM | 5307 | CB | ASP | A | 506 | 18.094 | 58.042 | −9.890 | 1.00 | 0.00 | C |
| ATOM | 5308 | CG | ASP | A | 506 | 18.215 | 59.491 | −10.334 | 1.00 | 0.00 | C |
| ATOM | 5309 | OD1 | ASP | A | 506 | 19.222 | 60.127 | −9.973 | 1.00 | 0.00 | O |
| ATOM | 5310 | OD2 | ASP | A | 506 | 17.320 | 60.033 | −11.017 | 1.00 | 0.00 | O1− |
| ATOM | 5311 | H | ASP | A | 506 | 17.429 | 55.871 | −8.940 | 1.00 | 0.00 | H |
| ATOM | 5312 | HA | ASP | A | 506 | 16.022 | 58.062 | −9.912 | 1.00 | 0.00 | H |
| ATOM | 5313 | 1HB | ASP | A | 506 | 18.210 | 58.037 | −8.806 | 1.00 | 0.00 | H |
| ATOM | 5314 | 2HB | ASP | A | 506 | 18.926 | 57.505 | −10.346 | 1.00 | 0.00 | H |
| ATOM | 5315 | N | ILE | A | 507 | 16.372 | 58.120 | −12.522 | 1.00 | 0.00 | N |
| ATOM | 5316 | CA | ILE | A | 507 | 15.978 | 58.094 | −13.909 | 1.00 | 0.00 | C |
| ATOM | 5317 | C | ILE | A | 507 | 16.858 | 59.059 | −14.704 | 1.00 | 0.00 | C |
| ATOM | 5318 | O | ILE | A | 507 | 17.087 | 58.823 | −15.887 | 1.00 | 0.00 | O |
| ATOM | 5319 | CB | ILE | A | 507 | 14.483 | 58.445 | −14.000 | 1.00 | 0.00 | C |
| ATOM | 5320 | CG1 | ILE | A | 507 | 14.206 | 59.860 | −13.451 | 1.00 | 0.00 | C |
| ATOM | 5321 | CG2 | ILE | A | 507 | 13.644 | 57.388 | −13.264 | 1.00 | 0.00 | C |
| ATOM | 5322 | CD1 | ILE | A | 507 | 12.718 | 60.203 | −13.419 | 1.00 | 0.00 | C |
| ATOM | 5323 | H | ILE | A | 507 | 16.419 | 59.051 | −12.133 | 1.00 | 0.00 | H |
| ATOM | 5324 | HA | ILE | A | 507 | 16.043 | 57.073 | −14.286 | 1.00 | 0.00 | H |
| ATOM | 5325 | HB | ILE | A | 507 | 14.147 | 58.342 | −15.032 | 1.00 | 0.00 | H |
| ATOM | 5326 | 1HG1 | ILE | A | 507 | 14.587 | 59.936 | −12.433 | 1.00 | 0.00 | H |
| ATOM | 5327 | 2HG1 | ILE | A | 507 | 14.704 | 60.598 | −14.080 | 1.00 | 0.00 | H |
| ATOM | 5328 | 1HG2 | ILE | A | 507 | 12.588 | 57.647 | −13.335 | 1.00 | 0.00 | H |
| ATOM | 5329 | 2HG2 | ILE | A | 507 | 13.810 | 56.411 | −13.718 | 1.00 | 0.00 | H |
| ATOM | 5330 | 3HG2 | ILE | A | 507 | 13.940 | 57.354 | −12.215 | 1.00 | 0.00 | H |
| ATOM | 5331 | 1HD1 | ILE | A | 507 | 12.585 | 61.211 | −13.023 | 1.00 | 0.00 | H |
| ATOM | 5332 | 2HD1 | ILE | A | 507 | 12.311 | 60.154 | −14.429 | 1.00 | 0.00 | H |
| ATOM | 5333 | 3HD1 | ILE | A | 507 | 12.194 | 59.492 | −12.781 | 1.00 | 0.00 | H |
| ATOM | 5334 | N | SER | A | 508 | 17.358 | 60.120 | −14.059 | 1.00 | 0.00 | N |
| ATOM | 5335 | CA | SER | A | 508 | 18.363 | 61.038 | −14.579 | 1.00 | 0.00 | C |
| ATOM | 5336 | C | SER | A | 508 | 18.024 | 61.725 | −15.915 | 1.00 | 0.00 | C |
| ATOM | 5337 | O | SER | A | 508 | 17.802 | 62.937 | −15.955 | 1.00 | 0.00 | O |
| ATOM | 5338 | CB | SER | A | 508 | 19.724 | 60.337 | −14.579 | 1.00 | 0.00 | C |
| ATOM | 5339 | OG | SER | A | 508 | 20.046 | 59.964 | −13.251 | 1.00 | 0.00 | O |
| ATOM | 5340 | H | SER | A | 508 | 17.038 | 60.342 | −13.127 | 1.00 | 0.00 | H |
| ATOM | 5341 | HA | SER | A | 508 | 18.654 | 61.740 | −13.797 | 1.00 | 0.00 | H |
| ATOM | 5342 | 1HB | SER | A | 508 | 19.676 | 59.450 | −15.210 | 1.00 | 0.00 | H |
| ATOM | 5343 | 2HB | SER | A | 508 | 20.483 | 61.017 | −14.966 | 1.00 | 0.00 | H |
| ATOM | 5344 | HG | SER | A | 508 | 20.899 | 59.523 | −13.240 | 1.00 | 0.00 | H |
| ATOM | 5345 | N | ASN | A | 509 | 18.055 | 60.975 | −17.016 | 1.00 | 0.00 | N |
| ATOM | 5346 | CA | ASN | A | 509 | 17.932 | 61.459 | −18.388 | 1.00 | 0.00 | C |
| ATOM | 5347 | C | ASN | A | 509 | 16.466 | 61.433 | −18.827 | 1.00 | 0.00 | C |
| ATOM | 5348 | O | ASN | A | 509 | 16.152 | 61.579 | −20.005 | 1.00 | 0.00 | O |
| ATOM | 5349 | CB | ASN | A | 509 | 18.799 | 60.590 | −19.310 | 1.00 | 0.00 | C |
| ATOM | 5350 | CG | ASN | A | 509 | 18.809 | 61.076 | −20.761 | 1.00 | 0.00 | C |
| ATOM | 5351 | ND2 | ASN | A | 509 | 18.703 | 60.161 | −21.721 | 1.00 | 0.00 | N |
| ATOM | 5352 | OD1 | ASN | A | 509 | 18.930 | 62.266 | −21.042 | 1.00 | 0.00 | O |
| ATOM | 5353 | H | ASN | A | 509 | 18.172 | 59.974 | −16.942 | 1.00 | 0.00 | H |
| ATOM | 5354 | HA | ASN | A | 509 | 18.339 | 62.469 | −18.455 | 1.00 | 0.00 | H |
| ATOM | 5355 | 1HB | ASN | A | 509 | 19.829 | 60.597 | −18.954 | 1.00 | 0.00 | H |
| ATOM | 5356 | 2HB | ASN | A | 509 | 18.420 | 59.568 | −19.308 | 1.00 | 0.00 | H |
| ATOM | 5357 | 1HD2 | ASN | A | 509 | 18.706 | 60.444 | −22.690 | 1.00 | 0.00 | H |
| ATOM | 5358 | 2HD2 | ASN | A | 509 | 18.620 | 59.184 | −21.480 | 1.00 | 0.00 | H |
| ATOM | 5359 | N | VAL | A | 510 | 15.555 | 61.257 | −17.873 | 1.00 | 0.00 | N |
| ATOM | 5360 | CA | VAL | A | 510 | 14.177 | 61.671 | −18.060 | 1.00 | 0.00 | C |
| ATOM | 5361 | C | VAL | A | 510 | 14.204 | 63.194 | −18.059 | 1.00 | 0.00 | C |
| ATOM | 5362 | O | VAL | A | 510 | 14.877 | 63.801 | −17.226 | 1.00 | 0.00 | O |
| ATOM | 5363 | CB | VAL | A | 510 | 13.293 | 61.081 | −16.953 | 1.00 | 0.00 | C |
| ATOM | 5364 | CG1 | VAL | A | 510 | 11.907 | 61.734 | −16.850 | 1.00 | 0.00 | C |
| ATOM | 5365 | CG2 | VAL | A | 510 | 13.124 | 59.587 | −17.245 | 1.00 | 0.00 | C |
| ATOM | 5366 | H | VAL | A | 510 | 15.817 | 60.828 | −16.997 | 1.00 | 0.00 | H |
| ATOM | 5367 | HA | VAL | A | 510 | 13.791 | 61.244 | −18.986 | 1.00 | 0.00 | H |
| ATOM | 5368 | HB | VAL | A | 510 | 13.792 | 61.195 | −15.991 | 1.00 | 0.00 | H |
| ATOM | 5369 | 1HG1 | VAL | A | 510 | 11.342 | 61.263 | −16.045 | 1.00 | 0.00 | H |
| ATOM | 5370 | 2HG1 | VAL | A | 510 | 12.020 | 62.797 | −16.639 | 1.00 | 0.00 | H |
| ATOM | 5371 | 3HG1 | VAL | A | 510 | 11.373 | 61.605 | −17.791 | 1.00 | 0.00 | H |
| ATOM | 5372 | 1HG2 | VAL | A | 510 | 12.499 | 59.135 | −16.475 | 1.00 | 0.00 | H |
| ATOM | 5373 | 2HG2 | VAL | A | 510 | 12.651 | 59.458 | −18.218 | 1.00 | 0.00 | H |
| ATOM | 5374 | 3HG2 | VAL | A | 510 | 14.101 | 59.104 | −17.249 | 1.00 | 0.00 | H |
| ATOM | 5375 | N | LYS | A | 511 | 13.523 | 63.801 | −19.022 | 1.00 | 0.00 | N |
| ATOM | 5376 | CA | LYS | A | 511 | 13.549 | 65.225 | −19.316 | 1.00 | 0.00 | C |
| ATOM | 5377 | C | LYS | A | 511 | 12.113 | 65.667 | −19.586 | 1.00 | 0.00 | C |
| ATOM | 5378 | O | LYS | A | 511 | 11.861 | 66.612 | −20.328 | 1.00 | 0.00 | O |
| ATOM | 5379 | CB | LYS | A | 511 | 14.485 | 65.465 | −20.506 | 1.00 | 0.00 | C |
| ATOM | 5380 | CG | LYS | A | 511 | 15.933 | 65.190 | −20.073 | 1.00 | 0.00 | C |
| ATOM | 5381 | CD | LYS | A | 511 | 16.934 | 65.243 | −21.226 | 1.00 | 0.00 | C |
| ATOM | 5382 | CE | LYS | A | 511 | 16.615 | 64.190 | −22.293 | 1.00 | 0.00 | C |
| ATOM | 5383 | NZ | LYS | A | 511 | 17.819 | 63.776 | −23.024 | 1.00 | 0.00 | N1+ |
| ATOM | 5384 | H | LYS | A | 511 | 12.920 | 63.265 | −19.629 | 1.00 | 0.00 | H |
| ATOM | 5385 | HA | LYS | A | 511 | 14.004 | 65.761 | −18.483 | 1.00 | 0.00 | H |
| ATOM | 5386 | 1HB | LYS | A | 511 | 14.215 | 64.796 | −21.322 | 1.00 | 0.00 | H |
| ATOM | 5387 | 2HB | LYS | A | 511 | 14.393 | 66.499 | −20.839 | 1.00 | 0.00 | H |
| ATOM | 5388 | 1HG | LYS | A | 511 | 16.241 | 65.935 | −19.340 | 1.00 | 0.00 | H |
| ATOM | 5389 | 2HG | LYS | A | 511 | 15.998 | 64.197 | −19.630 | 1.00 | 0.00 | H |
| ATOM | 5390 | 1HD | LYS | A | 511 | 16.901 | 66.228 | −21.692 | 1.00 | 0.00 | H |
| ATOM | 5391 | 2HD | LYS | A | 511 | 17.937 | 65.055 | −20.845 | 1.00 | 0.00 | H |
| ATOM | 5392 | 1HE | LYS | A | 511 | 16.183 | 63.309 | −21.817 | 1.00 | 0.00 | H |
| ATOM | 5393 | 2HE | LYS | A | 511 | 15.903 | 64.601 | −23.009 | 1.00 | 0.00 | H |
| ATOM | 5394 | 1HZ | LYS | A | 511 | 17.572 | 63.084 | −23.717 | 1.00 | 0.00 | H |
| ATOM | 5395 | 2HZ | LYS | A | 511 | 18.228 | 64.579 | −23.482 | 1.00 | 0.00 | H |
| ATOM | 5396 | 3HZ | LYS | A | 511 | 18.487 | 63.382 | −22.378 | 1.00 | 0.00 | H |
| ATOM | 5397 | N | HIS | A | 512 | 11.168 | 64.954 | −18.976 | 1.00 | 0.00 | N |
| ATOM | 5398 | CA | HIS | A | 512 | 9.847 | 65.473 | −18.714 | 1.00 | 0.00 | C |
| ATOM | 5399 | C | HIS | A | 512 | 9.274 | 64.706 | −17.523 | 1.00 | 0.00 | C |
| ATOM | 5400 | O | HIS | A | 512 | 9.035 | 63.502 | −17.613 | 1.00 | 0.00 | O |
| ATOM | 5401 | CB | HIS | A | 512 | 8.941 | 65.376 | −19.950 | 1.00 | 0.00 | C |
| ATOM | 5402 | CG | HIS | A | 512 | 7.733 | 66.275 | −19.858 | 1.00 | 0.00 | C |
| ATOM | 5403 | CD2 | HIS | A | 512 | 7.419 | 67.299 | −18.994 | 1.00 | 0.00 | C |
| ATOM | 5404 | ND1 | HIS | A | 512 | 6.667 | 66.213 | −20.753 | 1.00 | 0.00 | N |
| ATOM | 5405 | CE1 | HIS | A | 512 | 5.857 | 67.251 | −20.489 | 1.00 | 0.00 | C |
| ATOM | 5406 | NE2 | HIS | A | 512 | 6.261 | 67.925 | −19.415 | 1.00 | 0.00 | N |
| ATOM | 5407 | H | HIS | A | 512 | 11.363 | 64.009 | −18.677 | 1.00 | 0.00 | H |
| ATOM | 5408 | HA | HIS | A | 512 | 9.905 | 66.548 | −18.543 | 1.00 | 0.00 | H |
| ATOM | 5409 | 1HB | HIS | A | 512 | 9.505 | 65.664 | −20.837 | 1.00 | 0.00 | H |
| ATOM | 5410 | 2HB | HIS | A | 512 | 8.588 | 64.351 | −20.062 | 1.00 | 0.00 | H |
| ATOM | 5411 | HD2 | HIS | A | 512 | 8.051 | 67.500 | −18.143 | 1.00 | 0.00 | H |
| ATOM | 5412 | HD1 | HIS | A | 512 | 6.624 | 65.470 | −21.436 | 1.00 | 0.00 | H |
| ATOM | 5413 | HE1 | HIS | A | 512 | 5.004 | 67.445 | −21.121 | 1.00 | 0.00 | H |
| ATOM | 5414 | N | VAL | A | 513 | 9.065 | 65.394 | −16.407 | 1.00 | 0.00 | N |
| ATOM | 5415 | CA | VAL | A | 513 | 8.251 | 64.911 | −15.309 | 1.00 | 0.00 | C |
| ATOM | 5416 | C | VAL | A | 513 | 6.906 | 65.608 | −15.452 | 1.00 | 0.00 | C |
| ATOM | 5417 | O | VAL | A | 513 | 6.859 | 66.809 | −15.712 | 1.00 | 0.00 | O |
| ATOM | 5418 | CB | VAL | A | 513 | 8.923 | 65.201 | −13.955 | 1.00 | 0.00 | C |
| ATOM | 5419 | CG1 | VAL | A | 513 | 7.926 | 65.132 | −12.789 | 1.00 | 0.00 | C |
| ATOM | 5420 | CG2 | VAL | A | 513 | 10.025 | 64.163 | −13.723 | 1.00 | 0.00 | C |
| ATOM | 5421 | H | VAL | A | 513 | 9.485 | 66.306 | −16.290 | 1.00 | 0.00 | H |
| ATOM | 5422 | HA | VAL | A | 513 | 8.178 | 63.824 | −15.361 | 1.00 | 0.00 | H |
| ATOM | 5423 | HB | VAL | A | 513 | 9.360 | 66.200 | −13.973 | 1.00 | 0.00 | H |
| ATOM | 5424 | 1HG1 | VAL | A | 513 | 8.445 | 65.344 | −11.854 | 1.00 | 0.00 | H |
| ATOM | 5425 | 2HG1 | VAL | A | 513 | 7.137 | 65.868 | −12.940 | 1.00 | 0.00 | H |
| ATOM | 5426 | 3HG1 | VAL | A | 513 | 7.489 | 64.135 | −12.743 | 1.00 | 0.00 | H |
| ATOM | 5427 | 1HG2 | VAL | A | 513 | 10.511 | 64.357 | −12.766 | 1.00 | 0.00 | H |
| ATOM | 5428 | 2HG2 | VAL | A | 513 | 9.588 | 63.165 | −13.712 | 1.00 | 0.00 | H |
| ATOM | 5429 | 3HG2 | VAL | A | 513 | 10.762 | 64.229 | −14.524 | 1.00 | 0.00 | H |
| ATOM | 5430 | N | ILE | A | 514 | 5.826 | 64.854 | −15.289 | 1.00 | 0.00 | N |
| ATOM | 5431 | CA | ILE | A | 514 | 4.475 | 65.366 | −15.337 | 1.00 | 0.00 | C |
| ATOM | 5432 | C | ILE | A | 514 | 3.866 | 65.009 | −13.983 | 1.00 | 0.00 | C |
| ATOM | 5433 | O | ILE | A | 514 | 3.528 | 63.853 | −13.733 | 1.00 | 0.00 | O |
| ATOM | 5434 | CB | ILE | A | 514 | 3.728 | 64.733 | −16.522 | 1.00 | 0.00 | C |
| ATOM | 5435 | CG1 | ILE | A | 514 | 4.446 | 65.040 | −17.852 | 1.00 | 0.00 | C |
| ATOM | 5436 | CG2 | ILE | A | 514 | 2.290 | 65.270 | −16.560 | 1.00 | 0.00 | C |
| ATOM | 5437 | CD1 | ILE | A | 514 | 4.097 | 64.016 | −18.932 | 1.00 | 0.00 | C |
| ATOM | 5438 | H | ILE | A | 514 | 5.923 | 63.863 | −15.119 | 1.00 | 0.00 | H |
| ATOM | 5439 | HA | ILE | A | 514 | 4.500 | 66.439 | −15.527 | 1.00 | 0.00 | H |
| ATOM | 5440 | HB | ILE | A | 514 | 3.661 | 63.655 | −16.375 | 1.00 | 0.00 | H |
| ATOM | 5441 | 1HG1 | ILE | A | 514 | 4.148 | 66.027 | −18.207 | 1.00 | 0.00 | H |
| ATOM | 5442 | 2HG1 | ILE | A | 514 | 5.524 | 65.021 | −17.697 | 1.00 | 0.00 | H |
| ATOM | 5443 | 1HG2 | ILE | A | 514 | 1.757 | 64.823 | −17.400 | 1.00 | 0.00 | H |
| ATOM | 5444 | 2HG2 | ILE | A | 514 | 1.782 | 65.016 | −15.631 | 1.00 | 0.00 | H |
| ATOM | 5445 | 3HG2 | ILE | A | 514 | 2.310 | 66.353 | −16.679 | 1.00 | 0.00 | H |
| ATOM | 5446 | 1HD1 | ILE | A | 514 | 4.621 | 64.266 | −19.854 | 1.00 | 0.00 | H |
| ATOM | 5447 | 2HD1 | ILE | A | 514 | 4.399 | 63.022 | −18.601 | 1.00 | 0.00 | H |
| ATOM | 5448 | 3HD1 | ILE | A | 514 | 3.022 | 64.028 | −19.111 | 1.00 | 0.00 | H |
| ATOM | 5449 | N | ASN | A | 515 | 3.760 | 65.977 | −13.078 | 1.00 | 0.00 | N |
| ATOM | 5450 | CA | ASN | A | 515 | 3.193 | 65.709 | −11.767 | 1.00 | 0.00 | C |
| ATOM | 5451 | C | ASN | A | 515 | 1.682 | 65.684 | −11.938 | 1.00 | 0.00 | C |
| ATOM | 5452 | O | ASN | A | 515 | 1.066 | 66.724 | −12.165 | 1.00 | 0.00 | O |
| ATOM | 5453 | CB | ASN | A | 515 | 3.607 | 66.762 | −10.733 | 1.00 | 0.00 | C |
| ATOM | 5454 | CG | ASN | A | 515 | 5.102 | 66.749 | −10.450 | 1.00 | 0.00 | C |
| ATOM | 5455 | ND2 | ASN | A | 515 | 5.750 | 67.908 | −10.451 | 1.00 | 0.00 | N |
| ATOM | 5456 | OD1 | ASN | A | 515 | 5.692 | 65.697 | −10.225 | 1.00 | 0.00 | O |
| ATOM | 5457 | H | ASN | A | 515 | 4.074 | 66.911 | −13.299 | 1.00 | 0.00 | H |
| ATOM | 5458 | HA | ASN | A | 515 | 3.578 | 64.760 | −11.391 | 1.00 | 0.00 | H |
| ATOM | 5459 | 1HB | ASN | A | 515 | 3.345 | 67.755 | −11.101 | 1.00 | 0.00 | H |
| ATOM | 5460 | 2HB | ASN | A | 515 | 3.087 | 66.574 | −9.794 | 1.00 | 0.00 | H |
| ATOM | 5461 | 1HD2 | ASN | A | 515 | 6.742 | 67.933 | −10.266 | 1.00 | 0.00 | H |
| ATOM | 5462 | 2HD2 | ASN | A | 515 | 5.250 | 68.766 | −10.636 | 1.00 | 0.00 | H |
| ATOM | 5463 | N | PHE | A | 516 | 1.093 | 64.497 | −11.835 | 1.00 | 0.00 | N |
| ATOM | 5464 | CA | PHE | A | 516 | −0.340 | 64.301 | −11.766 | 1.00 | 0.00 | C |
| ATOM | 5465 | C | PHE | A | 516 | −0.784 | 64.653 | −10.350 | 1.00 | 0.00 | C |
| ATOM | 5466 | O | PHE | A | 516 | −1.845 | 65.250 | −10.169 | 1.00 | 0.00 | O |
| ATOM | 5467 | CB | PHE | A | 516 | −0.642 | 62.839 | −12.106 | 1.00 | 0.00 | C |
| ATOM | 5468 | CG | PHE | A | 516 | −2.103 | 62.445 | −12.225 | 1.00 | 0.00 | C |
| ATOM | 5469 | CD1 | PHE | A | 516 | −2.658 | 62.151 | −13.487 | 1.00 | 0.00 | C |
| ATOM | 5470 | CD2 | PHE | A | 516 | −2.919 | 62.325 | −11.082 | 1.00 | 0.00 | C |
| ATOM | 5471 | CE1 | PHE | A | 516 | −3.998 | 61.742 | −13.605 | 1.00 | 0.00 | C |
| ATOM | 5472 | CE2 | PHE | A | 516 | −4.261 | 61.928 | −11.199 | 1.00 | 0.00 | C |
| ATOM | 5473 | CZ | PHE | A | 516 | −4.804 | 61.637 | −12.461 | 1.00 | 0.00 | C |
| ATOM | 5474 | H | PHE | A | 516 | 1.653 | 63.657 | −11.801 | 1.00 | 0.00 | H |
| ATOM | 5475 | HA | PHE | A | 516 | −0.831 | 64.937 | −12.502 | 1.00 | 0.00 | H |
| ATOM | 5476 | 1HB | PHE | A | 516 | −0.194 | 62.589 | −13.068 | 1.00 | 0.00 | H |
| ATOM | 5477 | 2HB | PHE | A | 516 | −0.228 | 62.192 | −11.333 | 1.00 | 0.00 | H |
| ATOM | 5478 | HD1 | PHE | A | 516 | −2.053 | 62.239 | −14.378 | 1.00 | 0.00 | H |
| ATOM | 5479 | HD2 | PHE | A | 516 | −2.514 | 62.540 | −10.104 | 1.00 | 0.00 | H |
| ATOM | 5480 | HE1 | PHE | A | 516 | −4.408 | 61.508 | −14.577 | 1.00 | 0.00 | H |
| ATOM | 5481 | HE2 | PHE | A | 516 | −4.879 | 61.845 | −10.318 | 1.00 | 0.00 | H |
| ATOM | 5482 | HZ | PHE | A | 516 | −5.837 | 61.334 | −12.553 | 1.00 | 0.00 | H |
| ATOM | 5483 | N | ASP | A | 517 | 0.049 | 64.288 | −9.373 | 1.00 | 0.00 | N |
| ATOM | 5484 | CA | ASP | A | 517 | −0.114 | 64.659 | −7.978 | 1.00 | 0.00 | C |
| ATOM | 5485 | C | ASP | A | 517 | 1.172 | 65.377 | −7.568 | 1.00 | 0.00 | C |
| ATOM | 5486 | O | ASP | A | 517 | 2.277 | 64.825 | −7.655 | 1.00 | 0.00 | O |
| ATOM | 5487 | CB | ASP | A | 517 | −0.334 | 63.437 | −7.074 | 1.00 | 0.00 | C |
| ATOM | 5488 | CG | ASP | A | 517 | −1.627 | 62.689 | −7.349 | 1.00 | 0.00 | C |
| ATOM | 5489 | OD1 | ASP | A | 517 | −2.702 | 63.325 | −7.287 | 1.00 | 0.00 | O |
| ATOM | 5490 | OD2 | ASP | A | 517 | −1.539 | 61.462 | −7.589 | 1.00 | 0.00 | O1− |
| ATOM | 5491 | H | ASP | A | 517 | 0.855 | 63.716 | −9.584 | 1.00 | 0.00 | H |
| ATOM | 5492 | HA | ASP | A | 517 | −0.976 | 65.319 | −7.876 | 1.00 | 0.00 | H |
| ATOM | 5493 | 1HB | ASP | A | 517 | 0.483 | 62.729 | −7.215 | 1.00 | 0.00 | H |
| ATOM | 5494 | 2HB | ASP | A | 517 | −0.362 | 63.756 | −6.032 | 1.00 | 0.00 | H |
| ATOM | 5495 | N | LEU | A | 518 | 1.035 | 66.612 | −7.100 | 1.00 | 0.00 | N |
| ATOM | 5496 | CA | LEU | A | 518 | 2.109 | 67.337 | −6.447 | 1.00 | 0.00 | C |
| ATOM | 5497 | C | LEU | A | 518 | 2.206 | 66.800 | −5.013 | 1.00 | 0.00 | C |
| ATOM | 5498 | O | LEU | A | 518 | 1.298 | 66.109 | −4.549 | 1.00 | 0.00 | O |
| ATOM | 5499 | CB | LEU | A | 518 | 1.809 | 68.841 | −6.546 | 1.00 | 0.00 | C |
| ATOM | 5500 | CG | LEU | A | 518 | 2.370 | 69.457 | −7.835 | 1.00 | 0.00 | C |
| ATOM | 5501 | CD1 | LEU | A | 518 | 1.668 | 70.796 | −8.069 | 1.00 | 0.00 | C |
| ATOM | 5502 | CD2 | LEU | A | 518 | 3.887 | 69.683 | −7.765 | 1.00 | 0.00 | C |
| ATOM | 5503 | H | LEU | A | 518 | 0.151 | 67.091 | −7.191 | 1.00 | 0.00 | H |
| ATOM | 5504 | HA | LEU | A | 518 | 3.026 | 67.228 | −7.025 | 1.00 | 0.00 | H |
| ATOM | 5505 | 1HB | LEU | A | 518 | 0.731 | 68.998 | −6.535 | 1.00 | 0.00 | H |
| ATOM | 5506 | 2HB | LEU | A | 518 | 2.259 | 69.358 | −5.699 | 1.00 | 0.00 | H |
| ATOM | 5507 | HG | LEU | A | 518 | 2.195 | 68.777 | −8.669 | 1.00 | 0.00 | H |
| ATOM | 5508 | 1HD1 | LEU | A | 518 | 2.051 | 71.253 | −8.982 | 1.00 | 0.00 | H |
| ATOM | 5509 | 2HD1 | LEU | A | 518 | 0.595 | 70.631 | −8.169 | 1.00 | 0.00 | H |
| ATOM | 5510 | 3HD1 | LEU | A | 518 | 1.857 | 71.458 | −7.225 | 1.00 | 0.00 | H |
| ATOM | 5511 | 1HD2 | LEU | A | 518 | 4.234 | 70.120 | −8.701 | 1.00 | 0.00 | H |
| ATOM | 5512 | 2HD2 | LEU | A | 518 | 4.116 | 70.359 | −6.941 | 1.00 | 0.00 | H |
| ATOM | 5513 | 3HD2 | LEU | A | 518 | 4.389 | 68.729 | −7.601 | 1.00 | 0.00 | H |
| ATOM | 5514 | N | PRO | A | 519 | 3.322 | 66.996 | −4.306 | 1.00 | 0.00 | N |
| ATOM | 5515 | CA | PRO | A | 519 | 3.506 | 66.379 | −3.004 | 1.00 | 0.00 | C |
| ATOM | 5516 | C | PRO | A | 519 | 2.497 | 66.882 | −1.968 | 1.00 | 0.00 | C |
| ATOM | 5517 | O | PRO | A | 519 | 2.177 | 68.067 | −1.917 | 1.00 | 0.00 | O |
| ATOM | 5518 | CB | PRO | A | 519 | 4.930 | 66.742 | −2.591 | 1.00 | 0.00 | C |
| ATOM | 5519 | CG | PRO | A | 519 | 5.199 | 68.048 | −3.325 | 1.00 | 0.00 | C |
| ATOM | 5520 | CD | PRO | A | 519 | 4.429 | 67.880 | −4.628 | 1.00 | 0.00 | C |
| ATOM | 5521 | HA | PRO | A | 519 | 3.394 | 65.299 | −3.093 | 1.00 | 0.00 | H |
| ATOM | 5522 | 1HB | PRO | A | 519 | 4.976 | 66.861 | −1.508 | 1.00 | 0.00 | H |
| ATOM | 5523 | 2HB | PRO | A | 519 | 5.612 | 65.949 | −2.898 | 1.00 | 0.00 | H |
| ATOM | 5524 | 1HG | PRO | A | 519 | 4.832 | 68.883 | −2.728 | 1.00 | 0.00 | H |
| ATOM | 5525 | 2HG | PRO | A | 519 | 6.271 | 68.160 | −3.486 | 1.00 | 0.00 | H |
| ATOM | 5526 | 1HD | PRO | A | 519 | 5.046 | 67.425 | −5.402 | 1.00 | 0.00 | H |
| ATOM | 5527 | 2HD | PRO | A | 519 | 4.040 | 68.833 | −4.988 | 1.00 | 0.00 | H |
| ATOM | 5528 | N | SER | A | 520 | 2.066 | 65.974 | −1.094 | 1.00 | 0.00 | N |
| ATOM | 5529 | CA | SER | A | 520 | 1.724 | 66.332 | 0.273 | 1.00 | 0.00 | C |
| ATOM | 5530 | C | SER | A | 520 | 2.975 | 66.140 | 1.141 | 1.00 | 0.00 | C |
| ATOM | 5531 | O | SER | A | 520 | 3.065 | 66.665 | 2.246 | 1.00 | 0.00 | O |
| ATOM | 5532 | CB | SER | A | 520 | 0.581 | 65.428 | 0.743 | 1.00 | 0.00 | C |
| ATOM | 5533 | OG | SER | A | 520 | −0.398 | 65.330 | −0.277 | 1.00 | 0.00 | O |
| ATOM | 5534 | H | SER | A | 520 | 1.970 | 65.009 | −1.374 | 1.00 | 0.00 | H |
| ATOM | 5535 | HA | SER | A | 520 | 1.387 | 67.368 | 0.305 | 1.00 | 0.00 | H |
| ATOM | 5536 | 1HB | SER | A | 520 | 0.972 | 64.435 | 0.967 | 1.00 | 0.00 | H |
| ATOM | 5537 | 2HB | SER | A | 520 | 0.129 | 65.850 | 1.640 | 1.00 | 0.00 | H |
| ATOM | 5538 | HG | SER | A | 520 | −1.114 | 64.763 | 0.019 | 1.00 | 0.00 | H |
| ATOM | 5539 | N | ASP | A | 521 | 3.948 | 65.382 | 0.628 | 1.00 | 0.00 | N |
| ATOM | 5540 | CA | ASP | A | 521 | 5.257 | 65.167 | 1.209 | 1.00 | 0.00 | C |
| ATOM | 5541 | C | ASP | A | 521 | 6.165 | 66.306 | 0.739 | 1.00 | 0.00 | C |
| ATOM | 5542 | O | ASP | A | 521 | 7.161 | 66.082 | 0.058 | 1.00 | 0.00 | O |
| ATOM | 5543 | CB | ASP | A | 521 | 5.768 | 63.764 | 0.823 | 1.00 | 0.00 | C |
| ATOM | 5544 | CG | ASP | A | 521 | 5.802 | 63.422 | −0.665 | 1.00 | 0.00 | C |
| ATOM | 5545 | OD1 | ASP | A | 521 | 4.922 | 63.893 | −1.423 | 1.00 | 0.00 | O |
| ATOM | 5546 | OD2 | ASP | A | 521 | 6.636 | 62.549 | −1.013 | 1.00 | 0.00 | O1− |
| ATOM | 5547 | H | ASP | A | 521 | 3.801 | 64.895 | −0.244 | 1.00 | 0.00 | H |
| ATOM | 5548 | HA | ASP | A | 521 | 5.167 | 65.095 | 2.293 | 1.00 | 0.00 | H |
| ATOM | 5549 | 1HB | ASP | A | 521 | 6.793 | 63.641 | 1.175 | 1.00 | 0.00 | H |
| ATOM | 5550 | 2HB | ASP | A | 521 | 5.133 | 63.007 | 1.283 | 1.00 | 0.00 | H |
| ATOM | 5551 | N | ILE | A | 522 | 5.752 | 67.528 | 1.097 | 1.00 | 0.00 | N |
| ATOM | 5552 | CA | ILE | A | 522 | 6.267 | 68.838 | 0.705 | 1.00 | 0.00 | C |
| ATOM | 5553 | C | ILE | A | 522 | 7.597 | 68.818 | −0.064 | 1.00 | 0.00 | C |
| ATOM | 5554 | O | ILE | A | 522 | 7.610 | 69.046 | −1.277 | 1.00 | 0.00 | O |
| ATOM | 5555 | CB | ILE | A | 522 | 6.208 | 69.823 | 1.898 | 1.00 | 0.00 | C |
| ATOM | 5556 | CG1 | ILE | A | 522 | 6.862 | 71.189 | 1.614 | 1.00 | 0.00 | C |
| ATOM | 5557 | CG2 | ILE | A | 522 | 6.776 | 69.262 | 3.213 | 1.00 | 0.00 | C |
| ATOM | 5558 | CD1 | ILE | A | 522 | 6.230 | 71.926 | 0.430 | 1.00 | 0.00 | C |
| ATOM | 5559 | H | ILE | A | 522 | 4.971 | 67.632 | 1.729 | 1.00 | 0.00 | H |
| ATOM | 5560 | HA | ILE | A | 522 | 5.466 | 69.426 | 0.258 | 1.00 | 0.00 | H |
| ATOM | 5561 | HB | ILE | A | 522 | 5.175 | 69.928 | 2.232 | 1.00 | 0.00 | H |
| ATOM | 5562 | 1HG1 | ILE | A | 522 | 6.763 | 71.829 | 2.490 | 1.00 | 0.00 | H |
| ATOM | 5563 | 2HG1 | ILE | A | 522 | 7.918 | 71.046 | 1.386 | 1.00 | 0.00 | H |
| ATOM | 5564 | 1HG2 | ILE | A | 522 | 6.695 | 70.016 | 3.996 | 1.00 | 0.00 | H |
| ATOM | 5565 | 2HG2 | ILE | A | 522 | 6.212 | 68.376 | 3.504 | 1.00 | 0.00 | H |
| ATOM | 5566 | 3HG2 | ILE | A | 522 | 7.823 | 68.996 | 3.073 | 1.00 | 0.00 | H |
| ATOM | 5567 | 1HD1 | ILE | A | 522 | 6.737 | 72.880 | 0.283 | 1.00 | 0.00 | H |
| ATOM | 5568 | 2HD1 | ILE | A | 522 | 6.330 | 71.320 | −0.471 | 1.00 | 0.00 | H |
| ATOM | 5569 | 3HD1 | ILE | A | 522 | 5.174 | 72.104 | 0.633 | 1.00 | 0.00 | H |
| ATOM | 5570 | N | GLU | A | 523 | 8.719 | 68.593 | 0.625 | 1.00 | 0.00 | N |
| ATOM | 5571 | CA | GLU | A | 523 | 10.041 | 68.763 | 0.051 | 1.00 | 0.00 | C |
| ATOM | 5572 | C | GLU | A | 523 | 10.263 | 67.861 | −1.169 | 1.00 | 0.00 | C |
| ATOM | 5573 | O | GLU | A | 523 | 11.162 | 68.129 | −1.967 | 1.00 | 0.00 | O |
| ATOM | 5574 | CB | GLU | A | 523 | 11.116 | 68.513 | 1.112 | 1.00 | 0.00 | C |
| ATOM | 5575 | CG | GLU | A | 523 | 11.144 | 69.566 | 2.228 | 1.00 | 0.00 | C |
| ATOM | 5576 | CD | GLU | A | 523 | 12.319 | 69.300 | 3.153 | 1.00 | 0.00 | C |
| ATOM | 5577 | OE1 | GLU | A | 523 | 12.074 | 68.849 | 4.289 | 1.00 | 0.00 | O |
| ATOM | 5578 | OE2 | GLU | A | 523 | 13.470 | 69.430 | 2.682 | 1.00 | 0.00 | O1− |
| ATOM | 5579 | H | GLU | A | 523 | 8.668 | 68.291 | 1.587 | 1.00 | 0.00 | H |
| ATOM | 5580 | HA | GLU | A | 523 | 10.197 | 69.812 | −0.201 | 1.00 | 0.00 | H |
| ATOM | 5581 | 1HB | GLU | A | 523 | 10.943 | 67.547 | 1.585 | 1.00 | 0.00 | H |
| ATOM | 5582 | 2HB | GLU | A | 523 | 12.100 | 68.516 | 0.642 | 1.00 | 0.00 | H |
| ATOM | 5583 | 1HG | GLU | A | 523 | 11.248 | 70.558 | 1.789 | 1.00 | 0.00 | H |
| ATOM | 5584 | 2HG | GLU | A | 523 | 10.217 | 69.516 | 2.798 | 1.00 | 0.00 | H |
| ATOM | 5585 | N | GLU | A | 524 | 9.454 | 66.810 | −1.359 | 1.00 | 0.00 | N |
| ATOM | 5586 | CA | GLU | A | 524 | 9.599 | 65.970 | −2.530 | 1.00 | 0.00 | C |
| ATOM | 5587 | C | GLU | A | 524 | 9.387 | 66.774 | −3.817 | 1.00 | 0.00 | C |
| ATOM | 5588 | O | GLU | A | 524 | 9.786 | 66.321 | −4.890 | 1.00 | 0.00 | O |
| ATOM | 5589 | CB | GLU | A | 524 | 8.717 | 64.706 | −2.494 | 1.00 | 0.00 | C |
| ATOM | 5590 | CG | GLU | A | 524 | 9.551 | 63.542 | −3.047 | 1.00 | 0.00 | C |
| ATOM | 5591 | CD | GLU | A | 524 | 8.787 | 62.331 | −3.517 | 1.00 | 0.00 | C |
| ATOM | 5592 | OE1 | GLU | A | 524 | 9.193 | 61.228 | −3.099 | 1.00 | 0.00 | O |
| ATOM | 5593 | OE2 | GLU | A | 524 | 7.954 | 62.482 | −4.430 | 1.00 | 0.00 | O1− |
| ATOM | 5594 | H | GLU | A | 524 | 8.735 | 66.597 | −0.682 | 1.00 | 0.00 | H |
| ATOM | 5595 | HA | GLU | A | 524 | 10.552 | 65.443 | −2.485 | 1.00 | 0.00 | H |
| ATOM | 5596 | 1HB | GLU | A | 524 | 8.415 | 64.502 | −1.467 | 1.00 | 0.00 | H |
| ATOM | 5597 | 2HB | GLU | A | 524 | 7.831 | 64.863 | −3.109 | 1.00 | 0.00 | H |
| ATOM | 5598 | 1HG | GLU | A | 524 | 10.127 | 63.884 | −3.907 | 1.00 | 0.00 | H |
| ATOM | 5599 | 2HG | GLU | A | 524 | 10.231 | 63.183 | −2.275 | 1.00 | 0.00 | H |
| ATOM | 5600 | N | TYR | A | 525 | 8.784 | 67.964 | −3.735 | 1.00 | 0.00 | N |
| ATOM | 5601 | CA | TYR | A | 525 | 8.741 | 68.901 | −4.846 | 1.00 | 0.00 | C |
| ATOM | 5602 | C | TYR | A | 525 | 10.147 | 69.057 | −5.426 | 1.00 | 0.00 | C |
| ATOM | 5603 | O | TYR | A | 525 | 10.339 | 68.960 | −6.638 | 1.00 | 0.00 | O |
| ATOM | 5604 | CB | TYR | A | 525 | 8.188 | 70.263 | −4.392 | 1.00 | 0.00 | C |
| ATOM | 5605 | CG | TYR | A | 525 | 8.024 | 71.332 | −5.465 | 1.00 | 0.00 | C |
| ATOM | 5606 | CD1 | TYR | A | 525 | 7.647 | 71.013 | −6.787 | 1.00 | 0.00 | C |
| ATOM | 5607 | CD2 | TYR | A | 525 | 8.213 | 72.688 | −5.135 | 1.00 | 0.00 | C |
| ATOM | 5608 | CE1 | TYR | A | 525 | 7.440 | 72.025 | −7.740 | 1.00 | 0.00 | C |
| ATOM | 5609 | CE2 | TYR | A | 525 | 7.999 | 73.701 | −6.086 | 1.00 | 0.00 | C |
| ATOM | 5610 | CZ | TYR | A | 525 | 7.590 | 73.379 | −7.392 | 1.00 | 0.00 | C |
| ATOM | 5611 | OH | TYR | A | 525 | 7.307 | 74.359 | −8.301 | 1.00 | 0.00 | O |
| ATOM | 5612 | H | TYR | A | 525 | 8.333 | 68.243 | −2.876 | 1.00 | 0.00 | H |
| ATOM | 5613 | HA | TYR | A | 525 | 8.040 | 68.541 | −5.599 | 1.00 | 0.00 | H |
| ATOM | 5614 | 1HB | TYR | A | 525 | 7.197 | 70.127 | −3.958 | 1.00 | 0.00 | H |
| ATOM | 5615 | 2HB | TYR | A | 525 | 8.855 | 70.695 | −3.646 | 1.00 | 0.00 | H |
| ATOM | 5616 | HD1 | TYR | A | 525 | 7.514 | 69.981 | −7.076 | 1.00 | 0.00 | H |
| ATOM | 5617 | HD2 | TYR | A | 525 | 8.526 | 72.963 | −4.139 | 1.00 | 0.00 | H |
| ATOM | 5618 | HE1 | TYR | A | 525 | 7.163 | 71.766 | −8.751 | 1.00 | 0.00 | H |
| ATOM | 5619 | HE2 | TYR | A | 525 | 8.148 | 74.736 | −5.815 | 1.00 | 0.00 | H |
| ATOM | 5620 | HH | TYR | A | 525 | 7.462 | 75.219 | −7.904 | 1.00 | 0.00 | H |
| ATOM | 5621 | N | VAL | A | 526 | 11.132 | 69.246 | −4.547 | 1.00 | 0.00 | N |
| ATOM | 5622 | CA | VAL | A | 526 | 12.514 | 69.488 | −4.916 | 1.00 | 0.00 | C |
| ATOM | 5623 | C | VAL | A | 526 | 13.075 | 68.255 | −5.641 | 1.00 | 0.00 | C |
| ATOM | 5624 | O | VAL | A | 526 | 14.034 | 68.357 | −6.406 | 1.00 | 0.00 | O |
| ATOM | 5625 | CB | VAL | A | 526 | 13.336 | 69.878 | −3.669 | 1.00 | 0.00 | C |
| ATOM | 5626 | CG1 | VAL | A | 526 | 14.749 | 70.340 | −4.055 | 1.00 | 0.00 | C |
| ATOM | 5627 | CG2 | VAL | A | 526 | 12.670 | 71.024 | −2.890 | 1.00 | 0.00 | C |
| ATOM | 5628 | H | VAL | A | 526 | 10.937 | 69.224 | −3.557 | 1.00 | 0.00 | H |
| ATOM | 5629 | HA | VAL | A | 526 | 12.579 | 70.401 | −5.506 | 1.00 | 0.00 | H |
| ATOM | 5630 | HB | VAL | A | 526 | 13.420 | 69.019 | −3.005 | 1.00 | 0.00 | H |
| ATOM | 5631 | 1HG1 | VAL | A | 526 | 15.302 | 70.608 | −3.155 | 1.00 | 0.00 | H |
| ATOM | 5632 | 2HG1 | VAL | A | 526 | 15.267 | 69.533 | −4.572 | 1.00 | 0.00 | H |
| ATOM | 5633 | 3HG1 | VAL | A | 526 | 14.681 | 71.208 | −4.711 | 1.00 | 0.00 | H |
| ATOM | 5634 | 1HG2 | VAL | A | 526 | 13.277 | 71.272 | −2.019 | 1.00 | 0.00 | H |
| ATOM | 5635 | 2HG2 | VAL | A | 526 | 12.584 | 71.900 | −3.533 | 1.00 | 0.00 | H |
| ATOM | 5636 | 3HG2 | VAL | A | 526 | 11.678 | 70.714 | −2.564 | 1.00 | 0.00 | H |
| ATOM | 5637 | N | HIS | A | 527 | 12.487 | 67.077 | −5.416 | 1.00 | 0.00 | N |
| ATOM | 5638 | CA | HIS | A | 527 | 12.940 | 65.855 | −6.049 | 1.00 | 0.00 | C |
| ATOM | 5639 | C | HIS | A | 527 | 12.162 | 65.632 | −7.342 | 1.00 | 0.00 | C |
| ATOM | 5640 | O | HIS | A | 527 | 12.693 | 65.041 | −8.277 | 1.00 | 0.00 | O |
| ATOM | 5641 | CB | HIS | A | 527 | 12.767 | 64.610 | −5.180 | 1.00 | 0.00 | C |
| ATOM | 5642 | CG | HIS | A | 527 | 12.974 | 64.734 | −3.701 | 1.00 | 0.00 | C |
| ATOM | 5643 | CD2 | HIS | A | 527 | 13.458 | 65.730 | −2.891 | 1.00 | 0.00 | C |
| ATOM | 5644 | ND1 | HIS | A | 527 | 12.631 | 63.693 | −2.847 | 1.00 | 0.00 | N |
| ATOM | 5645 | CE1 | HIS | A | 527 | 12.776 | 64.127 | −1.589 | 1.00 | 0.00 | C |
| ATOM | 5646 | NE2 | HIS | A | 527 | 13.310 | 65.342 | −1.576 | 1.00 | 0.00 | N |
| ATOM | 5647 | H | HIS | A | 527 | 11.699 | 67.018 | −4.787 | 1.00 | 0.00 | H |
| ATOM | 5648 | HA | HIS | A | 527 | 14.006 | 65.930 | −6.264 | 1.00 | 0.00 | H |
| ATOM | 5649 | 1HB | HIS | A | 527 | 11.752 | 64.229 | −5.287 | 1.00 | 0.00 | H |
| ATOM | 5650 | 2HB | HIS | A | 527 | 13.477 | 63.845 | −5.496 | 1.00 | 0.00 | H |
| ATOM | 5651 | HD2 | HIS | A | 527 | 13.867 | 66.630 | −3.327 | 1.00 | 0.00 | H |
| ATOM | 5652 | HD1 | HIS | A | 527 | 12.336 | 62.804 | −3.227 | 1.00 | 0.00 | H |
| ATOM | 5653 | HE1 | HIS | A | 527 | 12.472 | 63.502 | −0.763 | 1.00 | 0.00 | H |
| ATOM | 5654 | N | ARG | A | 528 | 10.894 | 66.035 | −7.405 | 1.00 | 0.00 | N |
| ATOM | 5655 | CA | ARG | A | 528 | 10.089 | 65.890 | −8.610 | 1.00 | 0.00 | C |
| ATOM | 5656 | C | ARG | A | 528 | 10.631 | 66.818 | −9.702 | 1.00 | 0.00 | C |
| ATOM | 5657 | O | ARG | A | 528 | 10.547 | 66.491 | −10.882 | 1.00 | 0.00 | O |
| ATOM | 5658 | CB | ARG | A | 528 | 8.593 | 66.087 | −8.310 | 1.00 | 0.00 | C |
| ATOM | 5659 | CG | ARG | A | 528 | 8.089 | 64.910 | −7.458 | 1.00 | 0.00 | C |
| ATOM | 5660 | CD | ARG | A | 528 | 6.596 | 64.940 | −7.092 | 1.00 | 0.00 | C |
| ATOM | 5661 | NE | ARG | A | 528 | 6.356 | 64.007 | −5.979 | 1.00 | 0.00 | N |
| ATOM | 5662 | CZ | ARG | A | 528 | 5.292 | 63.932 | −5.174 | 1.00 | 0.00 | C |
| ATOM | 5663 | NH1 | ARG | A | 528 | 4.085 | 64.337 | −5.578 | 1.00 | 0.00 | N1+ |
| ATOM | 5664 | NH2 | ARG | A | 528 | 5.507 | 63.441 | −3.960 | 1.00 | 0.00 | N |
| ATOM | 5665 | H | ARG | A | 528 | 10.459 | 66.459 | −6.598 | 1.00 | 0.00 | H |
| ATOM | 5666 | HA | ARG | A | 528 | 10.035 | 64.837 | −8.887 | 1.00 | 0.00 | H |
| ATOM | 5667 | 1HB | ARG | A | 528 | 8.451 | 67.020 | −7.766 | 1.00 | 0.00 | H |
| ATOM | 5668 | 2HB | ARG | A | 528 | 8.037 | 66.125 | −9.247 | 1.00 | 0.00 | H |
| ATOM | 5669 | 1HG | ARG | A | 528 | 8.252 | 63.976 | −7.996 | 1.00 | 0.00 | H |
| ATOM | 5670 | 2HG | ARG | A | 528 | 8.633 | 64.885 | −6.514 | 1.00 | 0.00 | H |
| ATOM | 5671 | 1HD | ARG | A | 528 | 6.315 | 65.949 | −6.792 | 1.00 | 0.00 | H |
| ATOM | 5672 | 2HD | ARG | A | 528 | 6.004 | 64.640 | −7.957 | 1.00 | 0.00 | H |
| ATOM | 5673 | HE | ARG | A | 528 | 7.128 | 63.366 | −5.857 | 1.00 | 0.00 | H |
| ATOM | 5674 | 1HH1 | ARG | A | 528 | 3.295 | 64.271 | −4.953 | 1.00 | 0.00 | H |
| ATOM | 5675 | 2HH1 | ARG | A | 528 | 3.962 | 64.709 | −6.509 | 1.00 | 0.00 | H |
| ATOM | 5676 | 1HH2 | ARG | A | 528 | 4.742 | 63.359 | −3.306 | 1.00 | 0.00 | H |
| ATOM | 5677 | 2HH2 | ARG | A | 528 | 6.435 | 63.148 | −3.691 | 1.00 | 0.00 | H |
| ATOM | 5678 | N | ILE | A | 529 | 11.272 | 67.921 | −9.309 | 1.00 | 0.00 | N |
| ATOM | 5679 | CA | ILE | A | 529 | 12.034 | 68.783 | −10.212 | 1.00 | 0.00 | C |
| ATOM | 5680 | C | ILE | A | 529 | 13.512 | 68.349 | −10.208 | 1.00 | 0.00 | C |
| ATOM | 5681 | O | ILE | A | 529 | 14.406 | 69.061 | −10.671 | 1.00 | 0.00 | O |
| ATOM | 5682 | CB | ILE | A | 529 | 11.833 | 70.263 | −9.827 | 1.00 | 0.00 | C |
| ATOM | 5683 | CG1 | ILE | A | 529 | 12.606 | 70.629 | −8.547 | 1.00 | 0.00 | C |
| ATOM | 5684 | CG2 | ILE | A | 529 | 10.332 | 70.566 | −9.691 | 1.00 | 0.00 | C |
| ATOM | 5685 | CD1 | ILE | A | 529 | 12.333 | 72.045 | −8.037 | 1.00 | 0.00 | C |
| ATOM | 5686 | H | ILE | A | 529 | 11.246 | 68.202 | −8.339 | 1.00 | 0.00 | H |
| ATOM | 5687 | HA | ILE | A | 529 | 11.578 | 68.767 | −11.202 | 1.00 | 0.00 | H |
| ATOM | 5688 | HB | ILE | A | 529 | 12.112 | 70.899 | −10.666 | 1.00 | 0.00 | H |
| ATOM | 5689 | 1HG1 | ILE | A | 529 | 12.331 | 69.941 | −7.747 | 1.00 | 0.00 | H |
| ATOM | 5690 | 2HG1 | ILE | A | 529 | 13.677 | 70.556 | −8.736 | 1.00 | 0.00 | H |
| ATOM | 5691 | 1HG2 | ILE | A | 529 | 10.195 | 71.613 | −9.419 | 1.00 | 0.00 | H |
| ATOM | 5692 | 2HG2 | ILE | A | 529 | 9.834 | 70.371 | −10.641 | 1.00 | 0.00 | H |
| ATOM | 5693 | 3HG2 | ILE | A | 529 | 9.900 | 69.931 | −8.917 | 1.00 | 0.00 | H |
| ATOM | 5694 | 1HD1 | ILE | A | 529 | 12.915 | 72.225 | −7.133 | 1.00 | 0.00 | H |
| ATOM | 5695 | 2HD1 | ILE | A | 529 | 12.618 | 72.768 | −8.801 | 1.00 | 0.00 | H |
| ATOM | 5696 | 3HD1 | ILE | A | 529 | 11.272 | 72.153 | −7.812 | 1.00 | 0.00 | H |
| ATOM | 5697 | N | GLY | A | 530 | 13.782 | 67.168 | −9.654 | 1.00 | 0.00 | N |
| ATOM | 5698 | CA | GLY | A | 530 | 15.089 | 66.725 | −9.216 | 1.00 | 0.00 | C |
| ATOM | 5699 | C | GLY | A | 530 | 15.520 | 65.525 | −10.047 | 1.00 | 0.00 | C |
| ATOM | 5700 | O | GLY | A | 530 | 16.499 | 65.605 | −10.790 | 1.00 | 0.00 | O |
| ATOM | 5701 | H | GLY | A | 530 | 13.041 | 66.496 | −9.511 | 1.00 | 0.00 | H |
| ATOM | 5702 | 1HA | GLY | A | 530 | 15.808 | 67.534 | −9.343 | 1.00 | 0.00 | H |
| ATOM | 5703 | 2HA | GLY | A | 530 | 15.043 | 66.443 | −8.164 | 1.00 | 0.00 | H |
| ATOM | 5704 | N | ARG | A | 531 | 14.771 | 64.417 | −9.965 | 1.00 | 0.00 | N |
| ATOM | 5705 | CA | ARG | A | 531 | 15.144 | 63.179 | −10.635 | 1.00 | 0.00 | C |
| ATOM | 5706 | C | ARG | A | 531 | 15.383 | 63.427 | −12.138 | 1.00 | 0.00 | C |
| ATOM | 5707 | O | ARG | A | 531 | 16.163 | 62.718 | −12.772 | 1.00 | 0.00 | O |
| ATOM | 5708 | CB | ARG | A | 531 | 14.124 | 62.032 | −10.440 | 1.00 | 0.00 | C |
| ATOM | 5709 | CG | ARG | A | 531 | 13.018 | 62.110 | −9.371 | 1.00 | 0.00 | C |
| ATOM | 5710 | CD | ARG | A | 531 | 13.469 | 62.000 | −7.904 | 1.00 | 0.00 | C |
| ATOM | 5711 | NE | ARG | A | 531 | 12.297 | 61.936 | −7.006 | 1.00 | 0.00 | N |
| ATOM | 5712 | CZ | ARG | A | 531 | 12.260 | 61.427 | −5.758 | 1.00 | 0.00 | C |
| ATOM | 5713 | NH1 | ARG | A | 531 | 13.400 | 61.062 | −5.160 | 1.00 | 0.00 | N1+ |
| ATOM | 5714 | NH2 | ARG | A | 531 | 11.066 | 61.359 | −5.158 | 1.00 | 0.00 | N |
| ATOM | 5715 | H | ARG | A | 531 | 13.918 | 64.423 | −9.424 | 1.00 | 0.00 | H |
| ATOM | 5716 | HA | ARG | A | 531 | 16.012 | 62.744 | −10.140 | 1.00 | 0.00 | H |
| ATOM | 5717 | 1HB | ARG | A | 531 | 13.565 | 61.881 | −11.364 | 1.00 | 0.00 | H |
| ATOM | 5718 | 2HB | ARG | A | 531 | 14.653 | 61.114 | −10.182 | 1.00 | 0.00 | H |
| ATOM | 5719 | 1HG | ARG | A | 531 | 12.501 | 63.066 | −9.451 | 1.00 | 0.00 | H |
| ATOM | 5720 | 2HG | ARG | A | 531 | 12.306 | 61.299 | −9.524 | 1.00 | 0.00 | H |
| ATOM | 5721 | 1HD | ARG | A | 531 | 14.064 | 61.096 | −7.774 | 1.00 | 0.00 | H |
| ATOM | 5722 | 2HD | ARG | A | 531 | 14.069 | 62.871 | −7.642 | 1.00 | 0.00 | H |
| ATOM | 5723 | HE | ARG | A | 531 | 11.462 | 62.327 | −7.417 | 1.00 | 0.00 | H |
| ATOM | 5724 | 1HH1 | ARG | A | 531 | 13.380 | 60.681 | −4.225 | 1.00 | 0.00 | H |
| ATOM | 5725 | 2HH1 | ARG | A | 531 | 14.281 | 61.168 | −5.643 | 1.00 | 0.00 | H |
| ATOM | 5726 | 1HH2 | ARG | A | 531 | 10.991 | 60.985 | −4.223 | 1.00 | 0.00 | H |
| ATOM | 5727 | 2HH2 | ARG | A | 531 | 10.239 | 61.682 | −5.640 | 1.00 | 0.00 | H |
| ATOM | 5728 | N | THR | A | 532 | 14.718 | 64.435 | −12.703 | 1.00 | 0.00 | N |
| ATOM | 5729 | CA | THR | A | 532 | 14.865 | 64.839 | −14.088 | 1.00 | 0.00 | C |
| ATOM | 5730 | C | THR | A | 532 | 15.942 | 65.926 | −14.198 | 1.00 | 0.00 | C |
| ATOM | 5731 | O | THR | A | 532 | 15.924 | 66.902 | −13.444 | 1.00 | 0.00 | O |
| ATOM | 5732 | CB | THR | A | 532 | 13.499 | 65.311 | −14.628 | 1.00 | 0.00 | C |
| ATOM | 5733 | CG2 | THR | A | 532 | 12.808 | 66.402 | −13.797 | 1.00 | 0.00 | C |
| ATOM | 5734 | OG1 | THR | A | 532 | 13.615 | 65.791 | −15.948 | 1.00 | 0.00 | O |
| ATOM | 5735 | H | THR | A | 532 | 14.060 | 64.973 | −12.158 | 1.00 | 0.00 | H |
| ATOM | 5736 | HA | THR | A | 532 | 15.098 | 63.967 | −14.699 | 1.00 | 0.00 | H |
| ATOM | 5737 | HB | THR | A | 532 | 12.825 | 64.459 | −14.709 | 1.00 | 0.00 | H |
| ATOM | 5738 | 1HG2 | THR | A | 532 | 11.856 | 66.664 | −14.260 | 1.00 | 0.00 | H |
| ATOM | 5739 | 2HG2 | THR | A | 532 | 12.630 | 66.032 | −12.787 | 1.00 | 0.00 | H |
| ATOM | 5740 | 3HG2 | THR | A | 532 | 13.445 | 67.285 | −13.753 | 1.00 | 0.00 | H |
| ATOM | 5741 | HG1 | THR | A | 532 | 12.755 | 66.079 | −16.262 | 1.00 | 0.00 | H |
| ATOM | 5742 | N | GLY | A | 533 | 16.869 | 65.784 | −15.151 | 1.00 | 0.00 | N |
| ATOM | 5743 | CA | GLY | A | 533 | 17.707 | 66.888 | −15.601 | 1.00 | 0.00 | C |
| ATOM | 5744 | C | GLY | A | 533 | 19.166 | 66.485 | −15.793 | 1.00 | 0.00 | C |
| ATOM | 5745 | O | GLY | A | 533 | 20.043 | 67.346 | −15.847 | 1.00 | 0.00 | O |
| ATOM | 5746 | H | GLY | A | 533 | 17.009 | 64.884 | −15.588 | 1.00 | 0.00 | H |
| ATOM | 5747 | 1HA | GLY | A | 533 | 17.337 | 67.260 | −16.556 | 1.00 | 0.00 | H |
| ATOM | 5748 | 2HA | GLY | A | 533 | 17.679 | 67.690 | −14.864 | 1.00 | 0.00 | H |
| ATOM | 5749 | N | ARG | A | 534 | 19.450 | 65.188 | −15.894 | 1.00 | 0.00 | N |
| ATOM | 5750 | CA | ARG | A | 534 | 20.799 | 64.674 | −16.071 | 1.00 | 0.00 | C |
| ATOM | 5751 | C | ARG | A | 534 | 20.769 | 63.645 | −17.202 | 1.00 | 0.00 | C |
| ATOM | 5752 | O | ARG | A | 534 | 20.855 | 62.449 | −16.961 | 1.00 | 0.00 | O |
| ATOM | 5753 | CB | ARG | A | 534 | 21.274 | 64.064 | −14.742 | 1.00 | 0.00 | C |
| ATOM | 5754 | CG | ARG | A | 534 | 21.518 | 65.121 | −13.648 | 1.00 | 0.00 | C |
| ATOM | 5755 | CD | ARG | A | 534 | 21.439 | 64.498 | −12.250 | 1.00 | 0.00 | C |
| ATOM | 5756 | NE | ARG | A | 534 | 20.069 | 64.025 | −11.975 | 1.00 | 0.00 | N |
| ATOM | 5757 | CZ | ARG | A | 534 | 19.744 | 62.903 | −11.320 | 1.00 | 0.00 | C |
| ATOM | 5758 | NH1 | ARG | A | 534 | 18.498 | 62.455 | −11.380 | 1.00 | 0.00 | N1+ |
| ATOM | 5759 | NH2 | ARG | A | 534 | 20.657 | 62.232 | −10.631 | 1.00 | 0.00 | N |
| ATOM | 5760 | H | ARG | A | 534 | 18.708 | 64.505 | −15.849 | 1.00 | 0.00 | H |
| ATOM | 5761 | HA | ARG | A | 534 | 21.476 | 65.499 | −16.291 | 1.00 | 0.00 | H |
| ATOM | 5762 | HXT | ARG | A | 534 | 20.671 | 63.985 | −18.222 | 1.00 | 0.00 | H |
| ATOM | 5763 | 1HB | ARG | A | 534 | 20.520 | 63.370 | −14.370 | 1.00 | 0.00 | H |
| ATOM | 5764 | 2HB | ARG | A | 534 | 22.211 | 63.530 | −14.901 | 1.00 | 0.00 | H |
| ATOM | 5765 | 1HG | ARG | A | 534 | 22.508 | 65.559 | −13.779 | 1.00 | 0.00 | H |
| ATOM | 5766 | 2HG | ARG | A | 534 | 20.763 | 65.904 | −13.724 | 1.00 | 0.00 | H |
| ATOM | 5767 | 1HD | ARG | A | 534 | 22.125 | 63.653 | −12.189 | 1.00 | 0.00 | H |
| ATOM | 5768 | 2HD | ARG | A | 534 | 21.713 | 65.243 | −11.503 | 1.00 | 0.00 | H |
| ATOM | 5769 | HE | ARG | A | 534 | 19.343 | 64.631 | −12.330 | 1.00 | 0.00 | H |
| ATOM | 5770 | 1HH1 | ARG | A | 534 | 18.241 | 61.610 | −10.889 | 1.00 | 0.00 | H |
| ATOM | 5771 | 2HH1 | ARG | A | 534 | 17.805 | 62.959 | −11.915 | 1.00 | 0.00 | H |
| ATOM | 5772 | 1HH2 | ARG | A | 534 | 20.400 | 61.387 | −10.141 | 1.00 | 0.00 | H |
| ATOM | 5773 | 2HH2 | ARG | A | 534 | 21.610 | 62.565 | −10.596 | 1.00 | 0.00 | H |
| ATOM | 5774 | N | ASN | A | 537 | 19.113 | 68.471 | −22.383 | 1.00 | 0.00 | N |
| ATOM | 5775 | CA | ASN | A | 537 | 19.295 | 67.883 | −21.045 | 1.00 | 0.00 | C |
| ATOM | 5776 | C | ASN | A | 537 | 18.159 | 68.315 | −20.113 | 1.00 | 0.00 | C |
| ATOM | 5777 | O | ASN | A | 537 | 17.958 | 67.724 | −19.060 | 1.00 | 0.00 | O |
| ATOM | 5778 | CB | ASN | A | 537 | 20.649 | 68.285 | −20.444 | 1.00 | 0.00 | C |
| ATOM | 5779 | CG | ASN | A | 537 | 20.894 | 67.602 | −19.094 | 1.00 | 0.00 | C |
| ATOM | 5780 | ND2 | ASN | A | 537 | 21.268 | 68.362 | −18.073 | 1.00 | 0.00 | N |
| ATOM | 5781 | OD1 | ASN | A | 537 | 20.753 | 66.389 | −18.964 | 1.00 | 0.00 | O |
| ATOM | 5782 | 1H | ASN | A | 537 | 19.868 | 68.178 | −22.986 | 1.00 | 0.00 | H |
| ATOM | 5783 | 2H | ASN | A | 537 | 18.233 | 68.161 | −22.770 | 1.00 | 0.00 | H |
| ATOM | 5784 | HA | ASN | A | 537 | 19.337 | 66.797 | −21.128 | 1.00 | 0.00 | H |
| ATOM | 5785 | 1HB | ASN | A | 537 | 21.449 | 67.995 | −21.125 | 1.00 | 0.00 | H |
| ATOM | 5786 | 2HB | ASN | A | 537 | 20.674 | 69.364 | −20.293 | 1.00 | 0.00 | H |
| ATOM | 5787 | 1HD2 | ASN | A | 537 | 21.438 | 67.945 | −17.169 | 1.00 | 0.00 | H |
| ATOM | 5788 | 2HD2 | ASN | A | 537 | 21.383 | 69.357 | −18.200 | 1.00 | 0.00 | H |
| ATOM | 5789 | N | LEU | A | 538 | 17.456 | 69.366 | −20.534 | 1.00 | 0.00 | N |
| ATOM | 5790 | CA | LEU | A | 538 | 16.408 | 70.109 | −19.836 | 1.00 | 0.00 | C |
| ATOM | 5791 | C | LEU | A | 538 | 15.661 | 69.278 | −18.780 | 1.00 | 0.00 | C |
| ATOM | 5792 | O | LEU | A | 538 | 14.968 | 68.318 | −19.115 | 1.00 | 0.00 | O |
| ATOM | 5793 | CB | LEU | A | 538 | 15.430 | 70.651 | −20.890 | 1.00 | 0.00 | C |
| ATOM | 5794 | CG | LEU | A | 538 | 14.528 | 71.782 | −20.370 | 1.00 | 0.00 | C |
| ATOM | 5795 | CD1 | LEU | A | 538 | 15.308 | 73.100 | −20.271 | 1.00 | 0.00 | C |
| ATOM | 5796 | CD2 | LEU | A | 538 | 13.354 | 71.977 | −21.336 | 1.00 | 0.00 | C |
| ATOM | 5797 | H | LEU | A | 538 | 17.624 | 69.749 | −21.453 | 1.00 | 0.00 | H |
| ATOM | 5798 | HA | LEU | A | 538 | 16.839 | 70.994 | −19.369 | 1.00 | 0.00 | H |
| ATOM | 5799 | 1HB | LEU | A | 538 | 15.990 | 71.046 | −21.738 | 1.00 | 0.00 | H |
| ATOM | 5800 | 2HB | LEU | A | 538 | 14.778 | 69.846 | −21.230 | 1.00 | 0.00 | H |
| ATOM | 5801 | HG | LEU | A | 538 | 14.155 | 71.524 | −19.379 | 1.00 | 0.00 | H |
| ATOM | 5802 | 1HD1 | LEU | A | 538 | 14.649 | 73.886 | −19.901 | 1.00 | 0.00 | H |
| ATOM | 5803 | 2HD1 | LEU | A | 538 | 16.146 | 72.977 | −19.585 | 1.00 | 0.00 | H |
| ATOM | 5804 | 3HD1 | LEU | A | 538 | 15.683 | 73.376 | −21.257 | 1.00 | 0.00 | H |
| ATOM | 5805 | 1HD2 | LEU | A | 538 | 12.711 | 72.778 | −20.971 | 1.00 | 0.00 | H |
| ATOM | 5806 | 2HD2 | LEU | A | 538 | 13.734 | 72.238 | −22.324 | 1.00 | 0.00 | H |
| ATOM | 5807 | 3HD2 | LEU | A | 538 | 12.779 | 71.053 | −21.401 | 1.00 | 0.00 | H |
| ATOM | 5808 | N | GLY | A | 539 | 15.772 | 69.661 | −17.509 | 1.00 | 0.00 | N |
| ATOM | 5809 | CA | GLY | A | 539 | 15.097 | 68.978 | −16.420 | 1.00 | 0.00 | C |
| ATOM | 5810 | C | GLY | A | 539 | 13.700 | 69.564 | −16.275 | 1.00 | 0.00 | C |
| ATOM | 5811 | O | GLY | A | 539 | 13.451 | 70.359 | −15.372 | 1.00 | 0.00 | O |
| ATOM | 5812 | H | GLY | A | 539 | 16.345 | 70.458 | −17.270 | 1.00 | 0.00 | H |
| ATOM | 5813 | 1HA | GLY | A | 539 | 15.032 | 67.913 | −16.642 | 1.00 | 0.00 | H |
| ATOM | 5814 | 2HA | GLY | A | 539 | 15.658 | 69.122 | −15.496 | 1.00 | 0.00 | H |
| ATOM | 5815 | N | LEU | A | 540 | 12.811 | 69.179 | −17.189 | 1.00 | 0.00 | N |
| ATOM | 5816 | CA | LEU | A | 540 | 11.489 | 69.768 | −17.306 | 1.00 | 0.00 | C |
| ATOM | 5817 | C | LEU | A | 540 | 10.535 | 69.049 | −16.353 | 1.00 | 0.00 | C |
| ATOM | 5818 | O | LEU | A | 540 | 10.362 | 67.835 | −16.456 | 1.00 | 0.00 | O |
| ATOM | 5819 | CB | LEU | A | 540 | 11.015 | 69.671 | −18.767 | 1.00 | 0.00 | C |
| ATOM | 5820 | CG | LEU | A | 540 | 10.078 | 70.810 | −19.210 | 1.00 | 0.00 | C |
| ATOM | 5821 | CD1 | LEU | A | 540 | 9.571 | 70.509 | −20.625 | 1.00 | 0.00 | C |
| ATOM | 5822 | CD2 | LEU | A | 540 | 8.872 | 71.017 | −18.287 | 1.00 | 0.00 | C |
| ATOM | 5823 | H | LEU | A | 540 | 13.044 | 68.445 | −17.843 | 1.00 | 0.00 | H |
| ATOM | 5824 | HA | LEU | A | 540 | 11.546 | 70.835 | −17.090 | 1.00 | 0.00 | H |
| ATOM | 5825 | 1HB | LEU | A | 540 | 11.878 | 69.693 | −19.432 | 1.00 | 0.00 | H |
| ATOM | 5826 | 2HB | LEU | A | 540 | 10.470 | 68.738 | −18.912 | 1.00 | 0.00 | H |
| ATOM | 5827 | HG | LEU | A | 540 | 10.618 | 71.757 | −19.180 | 1.00 | 0.00 | H |
| ATOM | 5828 | 1HD1 | LEU | A | 540 | 8.906 | 71.309 | −20.951 | 1.00 | 0.00 | H |
| ATOM | 5829 | 2HD1 | LEU | A | 540 | 10.418 | 70.440 | −21.308 | 1.00 | 0.00 | H |
| ATOM | 5830 | 3HD1 | LEU | A | 540 | 9.029 | 69.564 | −20.624 | 1.00 | 0.00 | H |
| ATOM | 5831 | 1HD2 | LEU | A | 540 | 8.261 | 71.836 | −18.665 | 1.00 | 0.00 | H |
| ATOM | 5832 | 2HD2 | LEU | A | 540 | 8.277 | 70.104 | −18.256 | 1.00 | 0.00 | H |
| ATOM | 5833 | 3HD2 | LEU | A | 540 | 9.220 | 71.258 | −17.282 | 1.00 | 0.00 | H |
| ATOM | 5834 | N | ALA | A | 541 | 9.886 | 69.793 | −15.464 | 1.00 | 0.00 | N |
| ATOM | 5835 | CA | ALA | A | 541 | 8.820 | 69.309 | −14.612 | 1.00 | 0.00 | C |
| ATOM | 5836 | C | ALA | A | 541 | 7.579 | 70.160 | −14.871 | 1.00 | 0.00 | C |
| ATOM | 5837 | O | ALA | A | 541 | 7.505 | 71.310 | −14.440 | 1.00 | 0.00 | O |
| ATOM | 5838 | CB | ALA | A | 541 | 9.260 | 69.402 | −13.152 | 1.00 | 0.00 | C |
| ATOM | 5839 | H | ALA | A | 541 | 10.126 | 70.768 | −15.349 | 1.00 | 0.00 | H |
| ATOM | 5840 | HA | ALA | A | 541 | 8.628 | 68.259 | −14.831 | 1.00 | 0.00 | H |
| ATOM | 5841 | 1HB | ALA | A | 541 | 8.460 | 69.039 | −12.507 | 1.00 | 0.00 | H |
| ATOM | 5842 | 2HB | ALA | A | 541 | 10.152 | 68.795 | −13.002 | 1.00 | 0.00 | H |
| ATOM | 5843 | 3HB | ALA | A | 541 | 9.482 | 70.441 | −12.905 | 1.00 | 0.00 | H |
| ATOM | 5844 | N | THR | A | 542 | 6.591 | 69.584 | −15.543 | 1.00 | 0.00 | N |
| ATOM | 5845 | CA | THR | A | 542 | 5.274 | 70.175 | −15.663 | 1.00 | 0.00 | C |
| ATOM | 5846 | C | THR | A | 542 | 4.426 | 69.562 | −14.548 | 1.00 | 0.00 | C |
| ATOM | 5847 | O | THR | A | 542 | 4.615 | 68.399 | −14.194 | 1.00 | 0.00 | O |
| ATOM | 5848 | CB | THR | A | 542 | 4.704 | 69.876 | −17.060 | 1.00 | 0.00 | C |
| ATOM | 5849 | CG2 | THR | A | 542 | 3.350 | 70.555 | −17.290 | 1.00 | 0.00 | C |
| ATOM | 5850 | OG1 | THR | A | 542 | 5.594 | 70.348 | −18.055 | 1.00 | 0.00 | O |
| ATOM | 5851 | H | THR | A | 542 | 6.741 | 68.695 | −15.999 | 1.00 | 0.00 | H |
| ATOM | 5852 | HA | THR | A | 542 | 5.352 | 71.258 | −15.574 | 1.00 | 0.00 | H |
| ATOM | 5853 | HB | THR | A | 542 | 4.561 | 68.801 | −17.173 | 1.00 | 0.00 | H |
| ATOM | 5854 | 1HG2 | THR | A | 542 | 2.987 | 70.315 | −18.290 | 1.00 | 0.00 | H |
| ATOM | 5855 | 2HG2 | THR | A | 542 | 2.633 | 70.199 | −16.550 | 1.00 | 0.00 | H |
| ATOM | 5856 | 3HG2 | THR | A | 542 | 3.463 | 71.635 | −17.195 | 1.00 | 0.00 | H |
| ATOM | 5857 | HG1 | THR | A | 542 | 5.232 | 70.159 | −18.924 | 1.00 | 0.00 | H |
| ATOM | 5858 | N | SER | A | 543 | 3.491 | 70.319 | −13.986 | 1.00 | 0.00 | N |
| ATOM | 5859 | CA | SER | A | 543 | 2.614 | 69.820 | −12.949 | 1.00 | 0.00 | C |
| ATOM | 5860 | C | SER | A | 543 | 1.175 | 70.211 | −13.251 | 1.00 | 0.00 | C |
| ATOM | 5861 | O | SER | A | 543 | 0.886 | 71.384 | −13.493 | 1.00 | 0.00 | O |
| ATOM | 5862 | CB | SER | A | 543 | 3.064 | 70.397 | −11.605 | 1.00 | 0.00 | C |
| ATOM | 5863 | OG | SER | A | 543 | 4.466 | 70.238 | −11.445 | 1.00 | 0.00 | O |
| ATOM | 5864 | H | SER | A | 543 | 3.373 | 71.278 | −14.281 | 1.00 | 0.00 | H |
| ATOM | 5865 | HA | SER | A | 543 | 2.708 | 68.736 | −12.883 | 1.00 | 0.00 | H |
| ATOM | 5866 | 1HB | SER | A | 543 | 2.819 | 71.458 | −11.565 | 1.00 | 0.00 | H |
| ATOM | 5867 | 2HB | SER | A | 543 | 2.553 | 69.875 | −10.796 | 1.00 | 0.00 | H |
| ATOM | 5868 | HG | SER | A | 543 | 4.735 | 70.604 | −10.599 | 1.00 | 0.00 | H |
| ATOM | 5869 | N | PHE | A | 544 | 0.284 | 69.229 | −13.184 | 1.00 | 0.00 | N |
| ATOM | 5870 | CA | PHE | A | 544 | −1.113 | 69.500 | −12.939 | 1.00 | 0.00 | C |
| ATOM | 5871 | C | PHE | A | 544 | −1.252 | 69.831 | −11.449 | 1.00 | 0.00 | C |
| ATOM | 5872 | O | PHE | A | 544 | −0.302 | 69.671 | −10.677 | 1.00 | 0.00 | O |
| ATOM | 5873 | CB | PHE | A | 544 | −1.953 | 68.277 | −13.331 | 1.00 | 0.00 | C |
| ATOM | 5874 | CG | PHE | A | 544 | −1.812 | 67.809 | −14.772 | 1.00 | 0.00 | C |
| ATOM | 5875 | CD1 | PHE | A | 544 | −1.796 | 68.735 | −15.832 | 1.00 | 0.00 | C |
| ATOM | 5876 | CD2 | PHE | A | 544 | −1.727 | 66.436 | −15.081 | 1.00 | 0.00 | C |
| ATOM | 5877 | CE1 | PHE | A | 544 | −1.712 | 68.303 | −17.164 | 1.00 | 0.00 | C |
| ATOM | 5878 | CE2 | PHE | A | 544 | −1.649 | 65.996 | −16.413 | 1.00 | 0.00 | C |
| ATOM | 5879 | CZ | PHE | A | 544 | −1.647 | 66.932 | −17.459 | 1.00 | 0.00 | C |
| ATOM | 5880 | H | PHE | A | 544 | 0.579 | 68.270 | −13.305 | 1.00 | 0.00 | H |
| ATOM | 5881 | HA | PHE | A | 544 | −1.440 | 70.326 | −13.571 | 1.00 | 0.00 | H |
| ATOM | 5882 | 1HB | PHE | A | 544 | −1.672 | 67.428 | −12.708 | 1.00 | 0.00 | H |
| ATOM | 5883 | 2HB | PHE | A | 544 | −3.010 | 68.500 | −13.186 | 1.00 | 0.00 | H |
| ATOM | 5884 | HD1 | PHE | A | 544 | −1.847 | 69.794 | −15.625 | 1.00 | 0.00 | H |
| ATOM | 5885 | HD2 | PHE | A | 544 | −1.722 | 65.703 | −14.287 | 1.00 | 0.00 | H |
| ATOM | 5886 | HE1 | PHE | A | 544 | −1.697 | 69.025 | −17.967 | 1.00 | 0.00 | H |
| ATOM | 5887 | HE2 | PHE | A | 544 | −1.590 | 64.940 | −16.631 | 1.00 | 0.00 | H |
| ATOM | 5888 | HZ | PHE | A | 544 | −1.596 | 66.602 | −18.486 | 1.00 | 0.00 | H |
| ATOM | 5889 | N | PHE | A | 545 | −2.432 | 70.277 | −11.038 | 1.00 | 0.00 | N |
| ATOM | 5890 | CA | PHE | A | 545 | −2.714 | 70.737 | −9.692 | 1.00 | 0.00 | C |
| ATOM | 5891 | C | PHE | A | 545 | −4.229 | 70.679 | −9.535 | 1.00 | 0.00 | C |
| ATOM | 5892 | O | PHE | A | 545 | −4.950 | 70.862 | −10.517 | 1.00 | 0.00 | O |
| ATOM | 5893 | CB | PHE | A | 545 | −2.183 | 72.170 | −9.517 | 1.00 | 0.00 | C |
| ATOM | 5894 | CG | PHE | A | 545 | −2.466 | 72.826 | −8.178 | 1.00 | 0.00 | C |
| ATOM | 5895 | CD1 | PHE | A | 545 | −1.462 | 72.906 | −7.193 | 1.00 | 0.00 | C |
| ATOM | 5896 | CD2 | PHE | A | 545 | −3.730 | 73.387 | −7.902 | 1.00 | 0.00 | C |
| ATOM | 5897 | CE1 | PHE | A | 545 | −1.709 | 73.542 | −5.964 | 1.00 | 0.00 | C |
| ATOM | 5898 | CE2 | PHE | A | 545 | −3.984 | 74.007 | −6.668 | 1.00 | 0.00 | C |
| ATOM | 5899 | CZ | PHE | A | 545 | −2.971 | 74.094 | −5.701 | 1.00 | 0.00 | C |
| ATOM | 5900 | H | PHE | A | 545 | −3.210 | 70.311 | −11.681 | 1.00 | 0.00 | H |
| ATOM | 5901 | HA | PHE | A | 545 | −2.213 | 70.089 | −8.973 | 1.00 | 0.00 | H |
| ATOM | 5902 | 1HB | PHE | A | 545 | −1.099 | 72.172 | −9.631 | 1.00 | 0.00 | H |
| ATOM | 5903 | 2HB | PHE | A | 545 | −2.629 | 72.818 | −10.271 | 1.00 | 0.00 | H |
| ATOM | 5904 | HD1 | PHE | A | 545 | −0.489 | 72.474 | −7.377 | 1.00 | 0.00 | H |
| ATOM | 5905 | HD2 | PHE | A | 545 | −4.513 | 73.343 | −8.644 | 1.00 | 0.00 | H |
| ATOM | 5906 | HE1 | PHE | A | 545 | −0.927 | 73.607 | −5.223 | 1.00 | 0.00 | H |
| ATOM | 5907 | HE2 | PHE | A | 545 | −4.962 | 74.418 | −6.461 | 1.00 | 0.00 | H |
| ATOM | 5908 | HZ | PHE | A | 545 | −3.160 | 74.584 | −4.757 | 1.00 | 0.00 | H |
| ATOM | 5909 | N | ASN | A | 546 | −4.686 | 70.428 | −8.313 | 1.00 | 0.00 | N |
| ATOM | 5910 | CA | ASN | A | 546 | −6.076 | 70.428 | −7.889 | 1.00 | 0.00 | C |
| ATOM | 5911 | C | ASN | A | 546 | −6.100 | 70.285 | −6.367 | 1.00 | 0.00 | C |
| ATOM | 5912 | O | ASN | A | 546 | −5.037 | 70.124 | −5.760 | 1.00 | 0.00 | O |
| ATOM | 5913 | CB | ASN | A | 546 | −6.953 | 69.386 | −8.607 | 1.00 | 0.00 | C |
| ATOM | 5914 | CG | ASN | A | 546 | −6.495 | 67.932 | −8.593 | 1.00 | 0.00 | C |
| ATOM | 5915 | ND2 | ASN | A | 546 | −7.345 | 67.033 | −9.073 | 1.00 | 0.00 | N |
| ATOM | 5916 | OD1 | ASN | A | 546 | −5.371 | 67.589 | −8.225 | 1.00 | 0.00 | O |
| ATOM | 5917 | H | ASN | A | 546 | −4.040 | 70.209 | −7.568 | 1.00 | 0.00 | H |
| ATOM | 5918 | HA | ASN | A | 546 | −6.578 | 71.310 | −8.288 | 1.00 | 0.00 | H |
| ATOM | 5919 | 1HB | ASN | A | 546 | −7.945 | 69.374 | −8.156 | 1.00 | 0.00 | H |
| ATOM | 5920 | 2HB | ASN | A | 546 | −7.037 | 69.646 | −9.662 | 1.00 | 0.00 | H |
| ATOM | 5921 | 1HD2 | ASN | A | 546 | −7.091 | 66.055 | −9.088 | 1.00 | 0.00 | H |
| ATOM | 5922 | 2HD2 | ASN | A | 546 | −8.245 | 67.326 | −9.425 | 1.00 | 0.00 | H |
| ATOM | 5923 | N | GLU | A | 547 | −7.293 | 70.409 | −5.770 | 1.00 | 0.00 | N |
| ATOM | 5924 | CA | GLU | A | 547 | −7.569 | 70.667 | −4.357 | 1.00 | 0.00 | C |
| ATOM | 5925 | C | GLU | A | 547 | −6.532 | 70.087 | −3.385 | 1.00 | 0.00 | C |
| ATOM | 5926 | O | GLU | A | 547 | −6.104 | 70.764 | −2.453 | 1.00 | 0.00 | O |
| ATOM | 5927 | CB | GLU | A | 547 | −8.983 | 70.179 | −4.012 | 1.00 | 0.00 | C |
| ATOM | 5928 | CG | GLU | A | 547 | −9.452 | 70.722 | −2.656 | 1.00 | 0.00 | C |
| ATOM | 5929 | CD | GLU | A | 547 | −10.840 | 70.216 | −2.313 | 1.00 | 0.00 | C |
| ATOM | 5930 | OE1 | GLU | A | 547 | −10.988 | 68.977 | −2.261 | 1.00 | 0.00 | O |
| ATOM | 5931 | OE2 | GLU | A | 547 | −11.721 | 71.079 | −2.110 | 1.00 | 0.00 | O1− |
| ATOM | 5932 | H | GLU | A | 547 | −8.140 | 70.322 | −6.313 | 1.00 | 0.00 | H |
| ATOM | 5933 | HA | GLU | A | 547 | −7.697 | 71.739 | −4.201 | 1.00 | 0.00 | H |
| ATOM | 5934 | 1HB | GLU | A | 547 | −9.681 | 70.518 | −4.778 | 1.00 | 0.00 | H |
| ATOM | 5935 | 2HB | GLU | A | 547 | −8.992 | 69.090 | −3.969 | 1.00 | 0.00 | H |
| ATOM | 5936 | 1HG | GLU | A | 547 | −8.761 | 70.398 | −1.876 | 1.00 | 0.00 | H |
| ATOM | 5937 | 2HG | GLU | A | 547 | −9.477 | 71.811 | −2.688 | 1.00 | 0.00 | H |
| ATOM | 5938 | N | ARG | A | 548 | −6.150 | 68.826 | −3.599 | 1.00 | 0.00 | N |
| ATOM | 5939 | CA | ARG | A | 548 | −5.126 | 68.112 | −2.848 | 1.00 | 0.00 | C |
| ATOM | 5940 | C | ARG | A | 548 | −3.976 | 69.020 | −2.412 | 1.00 | 0.00 | C |
| ATOM | 5941 | O | ARG | A | 548 | −3.484 | 68.921 | −1.287 | 1.00 | 0.00 | O |
| ATOM | 5942 | CB | ARG | A | 548 | −4.538 | 66.994 | −3.721 | 1.00 | 0.00 | C |
| ATOM | 5943 | CG | ARG | A | 548 | −5.576 | 65.919 | −4.046 | 1.00 | 0.00 | C |
| ATOM | 5944 | CD | ARG | A | 548 | −5.013 | 64.893 | −5.034 | 1.00 | 0.00 | C |
| ATOM | 5945 | NE | ARG | A | 548 | −6.068 | 63.930 | −5.375 | 1.00 | 0.00 | N |
| ATOM | 5946 | CZ | ARG | A | 548 | −6.280 | 63.424 | −6.595 | 1.00 | 0.00 | C |
| ATOM | 5947 | NH1 | ARG | A | 548 | −5.351 | 63.480 | −7.542 | 1.00 | 0.00 | N1+ |
| ATOM | 5948 | NH2 | ARG | A | 548 | −7.455 | 62.853 | −6.844 | 1.00 | 0.00 | N |
| ATOM | 5949 | H | ARG | A | 548 | −6.582 | 68.285 | −4.335 | 1.00 | 0.00 | H |
| ATOM | 5950 | HA | ARG | A | 548 | −5.569 | 67.680 | −1.950 | 1.00 | 0.00 | H |
| ATOM | 5951 | 1HB | ARG | A | 548 | −4.179 | 67.416 | −4.660 | 1.00 | 0.00 | H |
| ATOM | 5952 | 2HB | ARG | A | 548 | −3.709 | 66.520 | −3.196 | 1.00 | 0.00 | H |
| ATOM | 5953 | 1HG | ARG | A | 548 | −5.863 | 65.402 | −3.131 | 1.00 | 0.00 | H |
| ATOM | 5954 | 2HG | ARG | A | 548 | −6.456 | 66.385 | −4.490 | 1.00 | 0.00 | H |
| ATOM | 5955 | 1HD | ARG | A | 548 | −4.676 | 65.403 | −5.937 | 1.00 | 0.00 | H |
| ATOM | 5956 | 2HD | ARG | A | 548 | −4.173 | 64.371 | −4.577 | 1.00 | 0.00 | H |
| ATOM | 5957 | HE | ARG | A | 548 | −6.657 | 63.652 | −4.603 | 1.00 | 0.00 | H |
| ATOM | 5958 | 1HH1 | ARG | A | 548 | −5.537 | 63.090 | −8.455 | 1.00 | 0.00 | H |
| ATOM | 5959 | 2HH1 | ARG | A | 548 | −4.459 | 63.912 | −7.349 | 1.00 | 0.00 | H |
| ATOM | 5960 | 1HH2 | ARG | A | 548 | −7.644 | 62.462 | −7.756 | 1.00 | 0.00 | H |
| ATOM | 5961 | 2HH2 | ARG | A | 548 | −8.159 | 62.810 | −6.121 | 1.00 | 0.00 | H |
| ATOM | 5962 | N | ASN | A | 549 | −3.494 | 69.846 | −3.337 | 1.00 | 0.00 | N |
| ATOM | 5963 | CA | ASN | A | 549 | −2.240 | 70.557 | −3.176 | 1.00 | 0.00 | C |
| ATOM | 5964 | C | ASN | A | 549 | −2.491 | 72.015 | −2.819 | 1.00 | 0.00 | C |
| ATOM | 5965 | O | ASN | A | 549 | −1.551 | 72.807 | −2.783 | 1.00 | 0.00 | O |
| ATOM | 5966 | CB | ASN | A | 549 | −1.382 | 70.394 | −4.431 | 1.00 | 0.00 | C |
| ATOM | 5967 | CG | ASN | A | 549 | −0.988 | 68.935 | −4.626 | 1.00 | 0.00 | C |
| ATOM | 5968 | ND2 | ASN | A | 549 | −0.090 | 68.405 | −3.804 | 1.00 | 0.00 | N |
| ATOM | 5969 | OD1 | ASN | A | 549 | −1.476 | 68.264 | −5.531 | 1.00 | 0.00 | O |
| ATOM | 5970 | H | ASN | A | 549 | −4.006 | 69.998 | −4.194 | 1.00 | 0.00 | H |
| ATOM | 5971 | HA | ASN | A | 549 | −1.619 | 70.038 | −2.446 | 1.00 | 0.00 | H |
| ATOM | 5972 | 1HB | ASN | A | 549 | −1.945 | 70.726 | −5.303 | 1.00 | 0.00 | H |
| ATOM | 5973 | 2HB | ASN | A | 549 | −0.477 | 70.993 | −4.333 | 1.00 | 0.00 | H |
| ATOM | 5974 | 1HD2 | ASN | A | 549 | 0.188 | 67.440 | −3.913 | 1.00 | 0.00 | H |
| ATOM | 5975 | 2HD2 | ASN | A | 549 | 0.316 | 68.967 | −3.069 | 1.00 | 0.00 | H |
| ATOM | 5976 | N | ILE | A | 550 | −3.733 | 72.367 | −2.469 | 1.00 | 0.00 | N |
| ATOM | 5977 | CA | ILE | A | 550 | −4.015 | 73.646 | −1.828 | 1.00 | 0.00 | C |
| ATOM | 5978 | C | ILE | A | 550 | −3.178 | 73.743 | −0.545 | 1.00 | 0.00 | C |
| ATOM | 5979 | O | ILE | A | 550 | −2.795 | 74.826 | −0.105 | 1.00 | 0.00 | O |
| ATOM | 5980 | CB | ILE | A | 550 | −5.525 | 73.816 | −1.570 | 1.00 | 0.00 | C |
| ATOM | 5981 | CG1 | ILE | A | 550 | −6.289 | 74.008 | −2.895 | 1.00 | 0.00 | C |
| ATOM | 5982 | CG2 | ILE | A | 550 | −5.841 | 74.969 | −0.606 | 1.00 | 0.00 | C |
| ATOM | 5983 | CD1 | ILE | A | 550 | −6.171 | 75.416 | −3.492 | 1.00 | 0.00 | C |
| ATOM | 5984 | H | ILE | A | 550 | −4.505 | 71.741 | −2.648 | 1.00 | 0.00 | H |
| ATOM | 5985 | HA | ILE | A | 550 | −3.828 | 74.456 | −2.533 | 1.00 | 0.00 | H |
| ATOM | 5986 | HB | ILE | A | 550 | −5.896 | 72.961 | −1.005 | 1.00 | 0.00 | H |
| ATOM | 5987 | 1HG1 | ILE | A | 550 | −5.904 | 73.312 | −3.641 | 1.00 | 0.00 | H |
| ATOM | 5988 | 2HG1 | ILE | A | 550 | −7.350 | 73.817 | −2.734 | 1.00 | 0.00 | H |
| ATOM | 5989 | 1HG2 | ILE | A | 550 | −6.919 | 75.039 | −0.463 | 1.00 | 0.00 | H |
| ATOM | 5990 | 2HG2 | ILE | A | 550 | −5.359 | 74.782 | 0.354 | 1.00 | 0.00 | H |
| ATOM | 5991 | 3HG2 | ILE | A | 550 | −5.468 | 75.904 | −1.023 | 1.00 | 0.00 | H |
| ATOM | 5992 | 1HD1 | ILE | A | 550 | −6.737 | 75.466 | −4.422 | 1.00 | 0.00 | H |
| ATOM | 5993 | 2HD1 | ILE | A | 550 | −6.569 | 76.145 | −2.786 | 1.00 | 0.00 | H |
| ATOM | 5994 | 3HD1 | ILE | A | 550 | −5.123 | 75.640 | −3.693 | 1.00 | 0.00 | H |
| ATOM | 5995 | N | ASN | A | 551 | −2.861 | 72.584 | 0.032 | 1.00 | 0.00 | N |
| ATOM | 5996 | CA | ASN | A | 551 | −2.063 | 72.446 | 1.238 | 1.00 | 0.00 | C |
| ATOM | 5997 | C | ASN | A | 551 | −0.641 | 72.992 | 1.070 | 1.00 | 0.00 | C |
| ATOM | 5998 | O | ASN | A | 551 | 0.089 | 73.044 | 2.054 | 1.00 | 0.00 | O |
| ATOM | 5999 | CB | ASN | A | 551 | −1.995 | 70.965 | 1.636 | 1.00 | 0.00 | C |
| ATOM | 6000 | CG | ASN | A | 551 | −3.335 | 70.449 | 2.144 | 1.00 | 0.00 | C |
| ATOM | 6001 | ND2 | ASN | A | 551 | −4.042 | 69.652 | 1.351 | 1.00 | 0.00 | N |
| ATOM | 6002 | OD1 | ASN | A | 551 | −3.753 | 70.775 | 3.250 | 1.00 | 0.00 | O |
| ATOM | 6003 | H | ASN | A | 551 | −3.182 | 71.715 | −0.371 | 1.00 | 0.00 | H |
| ATOM | 6004 | HA | ASN | A | 551 | −2.553 | 72.968 | 2.060 | 1.00 | 0.00 | H |
| ATOM | 6005 | 1HB | ASN | A | 551 | −1.707 | 70.369 | 0.770 | 1.00 | 0.00 | H |
| ATOM | 6006 | 2HB | ASN | A | 551 | −1.257 | 70.834 | 2.427 | 1.00 | 0.00 | H |
| ATOM | 6007 | 1HD2 | ASN | A | 551 | −4.935 | 69.294 | 1.661 | 1.00 | 0.00 | H |
| ATOM | 6008 | 2HD2 | ASN | A | 551 | −3.688 | 69.404 | 0.438 | 1.00 | 0.00 | H |
| ATOM | 6009 | N | ILE | A | 552 | −0.209 | 73.338 | −0.149 | 1.00 | 0.00 | N |
| ATOM | 6010 | CA | ILE | A | 552 | 1.137 | 73.842 | −0.406 | 1.00 | 0.00 | C |
| ATOM | 6011 | C | ILE | A | 552 | 1.083 | 75.020 | −1.387 | 1.00 | 0.00 | C |
| ATOM | 6012 | O | ILE | A | 552 | 1.973 | 75.188 | −2.224 | 1.00 | 0.00 | O |
| ATOM | 6013 | CB | ILE | A | 552 | 2.054 | 72.705 | −0.910 | 1.00 | 0.00 | C |
| ATOM | 6014 | CG1 | ILE | A | 552 | 1.516 | 72.051 | −2.197 | 1.00 | 0.00 | C |
| ATOM | 6015 | CG2 | ILE | A | 552 | 2.260 | 71.643 | 0.181 | 1.00 | 0.00 | C |
| ATOM | 6016 | CD1 | ILE | A | 552 | 2.609 | 71.292 | −2.952 | 1.00 | 0.00 | C |
| ATOM | 6017 | H | ILE | A | 552 | −0.824 | 73.257 | −0.945 | 1.00 | 0.00 | H |
| ATOM | 6018 | HA | ILE | A | 552 | 1.615 | 74.101 | 0.538 | 1.00 | 0.00 | H |
| ATOM | 6019 | HB | ILE | A | 552 | 3.063 | 73.090 | −1.058 | 1.00 | 0.00 | H |
| ATOM | 6020 | 1HG1 | ILE | A | 552 | 0.724 | 71.346 | −1.943 | 1.00 | 0.00 | H |
| ATOM | 6021 | 2HG1 | ILE | A | 552 | 1.118 | 72.821 | −2.857 | 1.00 | 0.00 | H |
| ATOM | 6022 | 1HG2 | ILE | A | 552 | 2.910 | 70.853 | −0.197 | 1.00 | 0.00 | H |
| ATOM | 6023 | 2HG2 | ILE | A | 552 | 2.721 | 72.103 | 1.055 | 1.00 | 0.00 | H |
| ATOM | 6024 | 3HG2 | ILE | A | 552 | 1.297 | 71.217 | 0.461 | 1.00 | 0.00 | H |
| ATOM | 6025 | 1HD1 | ILE | A | 552 | 2.187 | 70.846 | −3.852 | 1.00 | 0.00 | H |
| ATOM | 6026 | 2HD1 | ILE | A | 552 | 3.407 | 71.982 | −3.227 | 1.00 | 0.00 | H |
| ATOM | 6027 | 3HD1 | ILE | A | 552 | 3.014 | 70.506 | −2.313 | 1.00 | 0.00 | H |
| ATOM | 6028 | N | THR | A | 553 | 0.048 | 75.858 | −1.298 | 1.00 | 0.00 | N |
| ATOM | 6029 | CA | THR | A | 553 | −0.076 | 77.005 | −2.191 | 1.00 | 0.00 | C |
| ATOM | 6030 | C | THR | A | 553 | 0.860 | 78.137 | −1.740 | 1.00 | 0.00 | C |
| ATOM | 6031 | O | THR | A | 553 | 0.417 | 79.236 | −1.414 | 1.00 | 0.00 | O |
| ATOM | 6032 | CB | THR | A | 553 | −1.543 | 77.464 | −2.290 | 1.00 | 0.00 | C |
| ATOM | 6033 | CG2 | THR | A | 553 | −1.815 | 78.210 | −3.599 | 1.00 | 0.00 | C |
| ATOM | 6034 | OG1 | THR | A | 553 | −2.400 | 76.349 | −2.295 | 1.00 | 0.00 | O |
| ATOM | 6035 | H | THR | A | 553 | −0.668 | 75.705 | −0.603 | 1.00 | 0.00 | H |
| ATOM | 6036 | HA | THR | A | 553 | 0.104 | 76.687 | −3.218 | 1.00 | 0.00 | H |
| ATOM | 6037 | HB | THR | A | 553 | −1.759 | 78.167 | −1.486 | 1.00 | 0.00 | H |
| ATOM | 6038 | 1HG2 | THR | A | 553 | −2.860 | 78.518 | −3.632 | 1.00 | 0.00 | H |
| ATOM | 6039 | 2HG2 | THR | A | 553 | −1.175 | 79.091 | −3.655 | 1.00 | 0.00 | H |
| ATOM | 6040 | 3HG2 | THR | A | 553 | −1.603 | 77.553 | −4.443 | 1.00 | 0.00 | H |
| ATOM | 6041 | HG1 | THR | A | 553 | −3.311 | 76.645 | −2.356 | 1.00 | 0.00 | H |
| ATOM | 6042 | N | LYS | A | 554 | 2.170 | 77.897 | −1.770 | 1.00 | 0.00 | N |
| ATOM | 6043 | CA | LYS | A | 554 | 3.163 | 78.918 | −1.495 | 1.00 | 0.00 | C |
| ATOM | 6044 | C | LYS | A | 554 | 4.530 | 78.439 | −1.969 | 1.00 | 0.00 | C |
| ATOM | 6045 | O | LYS | A | 554 | 5.025 | 78.933 | −2.977 | 1.00 | 0.00 | O |
| ATOM | 6046 | CB | LYS | A | 554 | 3.153 | 79.379 | −0.022 | 1.00 | 0.00 | C |
| ATOM | 6047 | CG | LYS | A | 554 | 2.858 | 80.885 | 0.083 | 1.00 | 0.00 | C |
| ATOM | 6048 | CD | LYS | A | 554 | 4.001 | 81.717 | −0.522 | 1.00 | 0.00 | C |
| ATOM | 6049 | CE | LYS | A | 554 | 3.586 | 83.175 | −0.766 | 1.00 | 0.00 | C |
| ATOM | 6050 | NZ | LYS | A | 554 | 4.608 | 83.872 | −1.567 | 1.00 | 0.00 | N1+ |
| ATOM | 6051 | H | LYS | A | 554 | 2.509 | 76.971 | −1.990 | 1.00 | 0.00 | H |
| ATOM | 6052 | HA | LYS | A | 554 | 2.833 | 79.868 | −1.916 | 1.00 | 0.00 | H |
| ATOM | 6053 | 1HB | LYS | A | 554 | 2.384 | 78.833 | 0.525 | 1.00 | 0.00 | H |
| ATOM | 6054 | 2HB | LYS | A | 554 | 4.126 | 79.182 | 0.428 | 1.00 | 0.00 | H |
| ATOM | 6055 | 1HG | LYS | A | 554 | 1.938 | 81.113 | −0.455 | 1.00 | 0.00 | H |
| ATOM | 6056 | 2HG | LYS | A | 554 | 2.743 | 81.161 | 1.131 | 1.00 | 0.00 | H |
| ATOM | 6057 | 1HD | LYS | A | 554 | 4.852 | 81.716 | 0.159 | 1.00 | 0.00 | H |
| ATOM | 6058 | 2HD | LYS | A | 554 | 4.300 | 81.285 | −1.477 | 1.00 | 0.00 | H |
| ATOM | 6059 | 1HE | LYS | A | 554 | 2.638 | 83.198 | −1.304 | 1.00 | 0.00 | H |
| ATOM | 6060 | 2HE | LYS | A | 554 | 3.475 | 83.686 | 0.190 | 1.00 | 0.00 | H |
| ATOM | 6061 | 1HZ | LYS | A | 554 | 4.322 | 84.829 | −1.719 | 1.00 | 0.00 | H |
| ATOM | 6062 | 2HZ | LYS | A | 554 | 5.489 | 83.859 | −1.074 | 1.00 | 0.00 | H |
| ATOM | 6063 | 3HZ | LYS | A | 554 | 4.714 | 83.407 | −2.457 | 1.00 | 0.00 | H |
| ATOM | 6064 | N | ASP | A | 555 | 5.127 | 77.464 | −1.285 | 1.00 | 0.00 | N |
| ATOM | 6065 | CA | ASP | A | 555 | 6.433 | 76.886 | −1.592 | 1.00 | 0.00 | C |
| ATOM | 6066 | C | ASP | A | 555 | 6.515 | 76.528 | −3.075 | 1.00 | 0.00 | C |
| ATOM | 6067 | O | ASP | A | 555 | 7.537 | 76.713 | −3.738 | 1.00 | 0.00 | O |
| ATOM | 6068 | CB | ASP | A | 555 | 6.629 | 75.606 | −0.770 | 1.00 | 0.00 | C |
| ATOM | 6069 | CG | ASP | A | 555 | 6.448 | 75.832 | 0.720 | 1.00 | 0.00 | C |
| ATOM | 6070 | OD1 | ASP | A | 555 | 7.447 | 75.676 | 1.450 | 1.00 | 0.00 | O |
| ATOM | 6071 | OD2 | ASP | A | 555 | 5.298 | 76.159 | 1.092 | 1.00 | 0.00 | O1− |
| ATOM | 6072 | H | ASP | A | 555 | 4.672 | 77.062 | −0.477 | 1.00 | 0.00 | H |
| ATOM | 6073 | HA | ASP | A | 555 | 7.214 | 77.613 | −1.369 | 1.00 | 0.00 | H |
| ATOM | 6074 | 1HB | ASP | A | 555 | 5.902 | 74.857 | −1.084 | 1.00 | 0.00 | H |
| ATOM | 6075 | 2HB | ASP | A | 555 | 7.637 | 75.223 | −0.928 | 1.00 | 0.00 | H |
| ATOM | 6076 | N | LEU | A | 556 | 5.400 | 75.986 | −3.569 | 1.00 | 0.00 | N |
| ATOM | 6077 | CA | LEU | A | 556 | 5.162 | 75.593 | −4.944 | 1.00 | 0.00 | C |
| ATOM | 6078 | C | LEU | A | 556 | 5.563 | 76.699 | −5.931 | 1.00 | 0.00 | C |
| ATOM | 6079 | O | LEU | A | 556 | 5.988 | 76.408 | −7.052 | 1.00 | 0.00 | O |
| ATOM | 6080 | CB | LEU | A | 556 | 3.674 | 75.231 | −5.059 | 1.00 | 0.00 | C |
| ATOM | 6081 | CG | LEU | A | 556 | 3.219 | 74.698 | −6.425 | 1.00 | 0.00 | C |
| ATOM | 6082 | CD1 | LEU | A | 556 | 3.922 | 73.390 | −6.796 | 1.00 | 0.00 | C |
| ATOM | 6083 | CD2 | LEU | A | 556 | 1.709 | 74.448 | −6.354 | 1.00 | 0.00 | C |
| ATOM | 6084 | H | LEU | A | 556 | 4.612 | 75.811 | −2.962 | 1.00 | 0.00 | H |
| ATOM | 6085 | HA | LEU | A | 556 | 5.703 | 74.671 | −5.158 | 1.00 | 0.00 | H |
| ATOM | 6086 | 1HB | LEU | A | 556 | 3.432 | 74.454 | −4.334 | 1.00 | 0.00 | H |
| ATOM | 6087 | 2HB | LEU | A | 556 | 3.068 | 76.115 | −4.861 | 1.00 | 0.00 | H |
| ATOM | 6088 | HG | LEU | A | 556 | 3.445 | 75.434 | −7.197 | 1.00 | 0.00 | H |
| ATOM | 6089 | 1HD1 | LEU | A | 556 | 3.569 | 73.050 | −7.770 | 1.00 | 0.00 | H |
| ATOM | 6090 | 2HD1 | LEU | A | 556 | 4.998 | 73.554 | −6.838 | 1.00 | 0.00 | H |
| ATOM | 6091 | 3HD1 | LEU | A | 556 | 3.699 | 72.631 | −6.045 | 1.00 | 0.00 | H |
| ATOM | 6092 | 1HD2 | LEU | A | 556 | 1.357 | 74.068 | −7.313 | 1.00 | 0.00 | H |
| ATOM | 6093 | 2HD2 | LEU | A | 556 | 1.497 | 73.716 | −5.574 | 1.00 | 0.00 | H |
| ATOM | 6094 | 3HD2 | LEU | A | 556 | 1.196 | 75.382 | −6.124 | 1.00 | 0.00 | H |
| ATOM | 6095 | N | LEU | A | 557 | 5.395 | 77.955 | −5.517 | 1.00 | 0.00 | N |
| ATOM | 6096 | CA | LEU | A | 557 | 5.710 | 79.156 | −6.267 | 1.00 | 0.00 | C |
| ATOM | 6097 | C | LEU | A | 557 | 7.040 | 79.716 | −5.772 | 1.00 | 0.00 | C |
| ATOM | 6098 | O | LEU | A | 557 | 7.927 | 80.004 | −6.570 | 1.00 | 0.00 | O |
| ATOM | 6099 | CB | LEU | A | 557 | 4.599 | 80.197 | −6.054 | 1.00 | 0.00 | C |
| ATOM | 6100 | CG | LEU | A | 557 | 4.803 | 81.486 | −6.871 | 1.00 | 0.00 | C |
| ATOM | 6101 | CD1 | LEU | A | 557 | 4.322 | 81.314 | −8.317 | 1.00 | 0.00 | C |
| ATOM | 6102 | CD2 | LEU | A | 557 | 4.023 | 82.628 | −6.215 | 1.00 | 0.00 | C |
| ATOM | 6103 | H | LEU | A | 557 | 5.014 | 78.137 | −4.599 | 1.00 | 0.00 | H |
| ATOM | 6104 | HA | LEU | A | 557 | 5.787 | 78.913 | −7.327 | 1.00 | 0.00 | H |
| ATOM | 6105 | 1HB | LEU | A | 557 | 3.640 | 79.772 | −6.350 | 1.00 | 0.00 | H |
| ATOM | 6106 | 2HB | LEU | A | 557 | 4.562 | 80.480 | −5.002 | 1.00 | 0.00 | H |
| ATOM | 6107 | HG | LEU | A | 557 | 5.860 | 81.749 | −6.877 | 1.00 | 0.00 | H |
| ATOM | 6108 | 1HD1 | LEU | A | 557 | 4.481 | 82.243 | −8.865 | 1.00 | 0.00 | H |
| ATOM | 6109 | 2HD1 | LEU | A | 557 | 4.883 | 80.511 | −8.794 | 1.00 | 0.00 | H |
| ATOM | 6110 | 3HD1 | LEU | A | 557 | 3.261 | 81.067 | −8.320 | 1.00 | 0.00 | H |
| ATOM | 6111 | 1HD2 | LEU | A | 557 | 4.165 | 83.543 | −6.791 | 1.00 | 0.00 | H |
| ATOM | 6112 | 2HD2 | LEU | A | 557 | 2.963 | 82.377 | −6.188 | 1.00 | 0.00 | H |
| ATOM | 6113 | 3HD2 | LEU | A | 557 | 4.386 | 82.780 | −5.198 | 1.00 | 0.00 | H |
| ATOM | 6114 | N | ASP | A | 558 | 7.165 | 79.946 | −4.467 | 1.00 | 0.00 | N |
| ATOM | 6115 | CA | ASP | A | 558 | 8.219 | 80.799 | −3.942 | 1.00 | 0.00 | C |
| ATOM | 6116 | C | ASP | A | 558 | 9.590 | 80.164 | −4.180 | 1.00 | 0.00 | C |
| ATOM | 6117 | O | ASP | A | 558 | 10.591 | 80.870 | −4.293 | 1.00 | 0.00 | O |
| ATOM | 6118 | CB | ASP | A | 558 | 7.985 | 81.132 | −2.461 | 1.00 | 0.00 | C |
| ATOM | 6119 | CG | ASP | A | 558 | 6.951 | 82.228 | −2.263 | 1.00 | 0.00 | C |
| ATOM | 6120 | OD1 | ASP | A | 558 | 6.040 | 82.376 | −3.107 | 1.00 | 0.00 | O |
| ATOM | 6121 | OD2 | ASP | A | 558 | 7.007 | 82.938 | −1.234 | 1.00 | 0.00 | O1− |
| ATOM | 6122 | H | ASP | A | 558 | 6.519 | 79.523 | −3.816 | 1.00 | 0.00 | H |
| ATOM | 6123 | HA | ASP | A | 558 | 8.128 | 81.797 | −4.371 | 1.00 | 0.00 | H |
| ATOM | 6124 | 1HB | ASP | A | 558 | 7.633 | 80.243 | −1.939 | 1.00 | 0.00 | H |
| ATOM | 6125 | 2HB | ASP | A | 558 | 8.919 | 81.470 | −2.012 | 1.00 | 0.00 | H |
| ATOM | 6126 | N | LEU | A | 559 | 9.654 | 78.835 | −4.296 | 1.00 | 0.00 | N |
| ATOM | 6127 | CA | LEU | A | 559 | 10.892 | 78.148 | −4.630 | 1.00 | 0.00 | C |
| ATOM | 6128 | C | LEU | A | 559 | 11.444 | 78.650 | −5.972 | 1.00 | 0.00 | C |
| ATOM | 6129 | O | LEU | A | 559 | 12.655 | 78.634 | −6.201 | 1.00 | 0.00 | O |
| ATOM | 6130 | CB | LEU | A | 559 | 10.644 | 76.632 | −4.663 | 1.00 | 0.00 | C |
| ATOM | 6131 | CG | LEU | A | 559 | 11.890 | 75.789 | −4.989 | 1.00 | 0.00 | C |
| ATOM | 6132 | CD1 | LEU | A | 559 | 12.992 | 75.955 | −3.934 | 1.00 | 0.00 | C |
| ATOM | 6133 | CD2 | LEU | A | 559 | 11.495 | 74.312 | −5.075 | 1.00 | 0.00 | C |
| ATOM | 6134 | H | LEU | A | 559 | 8.826 | 78.275 | −4.150 | 1.00 | 0.00 | H |
| ATOM | 6135 | HA | LEU | A | 559 | 11.617 | 78.293 | −3.828 | 1.00 | 0.00 | H |
| ATOM | 6136 | 1HB | LEU | A | 559 | 10.283 | 76.301 | −3.689 | 1.00 | 0.00 | H |
| ATOM | 6137 | 2HB | LEU | A | 559 | 9.897 | 76.403 | −5.424 | 1.00 | 0.00 | H |
| ATOM | 6138 | HG | LEU | A | 559 | 12.283 | 76.082 | −5.963 | 1.00 | 0.00 | H |
| ATOM | 6139 | 1HD1 | LEU | A | 559 | 13.851 | 75.343 | −4.206 | 1.00 | 0.00 | H |
| ATOM | 6140 | 2HD1 | LEU | A | 559 | 13.293 | 77.002 | −3.885 | 1.00 | 0.00 | H |
| ATOM | 6141 | 3HD1 | LEU | A | 559 | 12.614 | 75.640 | −2.962 | 1.00 | 0.00 | H |
| ATOM | 6142 | 1HD2 | LEU | A | 559 | 12.375 | 73.712 | −5.306 | 1.00 | 0.00 | H |
| ATOM | 6143 | 2HD2 | LEU | A | 559 | 11.078 | 73.991 | −4.121 | 1.00 | 0.00 | H |
| ATOM | 6144 | 3HD2 | LEU | A | 559 | 10.750 | 74.179 | −5.860 | 1.00 | 0.00 | H |
| ATOM | 6145 | N | LEU | A | 560 | 10.563 | 79.077 | −6.878 | 1.00 | 0.00 | N |
| ATOM | 6146 | CA | LEU | A | 560 | 10.945 | 79.551 | −8.198 | 1.00 | 0.00 | C |
| ATOM | 6147 | C | LEU | A | 560 | 11.771 | 80.833 | −8.086 | 1.00 | 0.00 | C |
| ATOM | 6148 | O | LEU | A | 560 | 12.526 | 81.165 | −9.002 | 1.00 | 0.00 | O |
| ATOM | 6149 | CB | LEU | A | 560 | 9.702 | 79.811 | −9.062 | 1.00 | 0.00 | C |
| ATOM | 6150 | CG | LEU | A | 560 | 8.733 | 78.620 | −9.181 | 1.00 | 0.00 | C |
| ATOM | 6151 | CD1 | LEU | A | 560 | 7.471 | 79.093 | −9.901 | 1.00 | 0.00 | C |
| ATOM | 6152 | CD2 | LEU | A | 560 | 9.329 | 77.448 | −9.960 | 1.00 | 0.00 | C |
| ATOM | 6153 | H | LEU | A | 560 | 9.577 | 79.081 | −6.661 | 1.00 | 0.00 | H |
| ATOM | 6154 | HA | LEU | A | 560 | 11.515 | 78.777 | −8.712 | 1.00 | 0.00 | H |
| ATOM | 6155 | 1HB | LEU | A | 560 | 9.131 | 80.637 | −8.638 | 1.00 | 0.00 | H |
| ATOM | 6156 | 2HB | LEU | A | 560 | 10.011 | 80.066 | −10.076 | 1.00 | 0.00 | H |
| ATOM | 6157 | HG | LEU | A | 560 | 8.485 | 78.252 | −8.186 | 1.00 | 0.00 | H |
| ATOM | 6158 | 1HD1 | LEU | A | 560 | 6.772 | 78.262 | −9.995 | 1.00 | 0.00 | H |
| ATOM | 6159 | 2HD1 | LEU | A | 560 | 7.005 | 79.896 | −9.330 | 1.00 | 0.00 | H |
| ATOM | 6160 | 3HD1 | LEU | A | 560 | 7.734 | 79.460 | −10.894 | 1.00 | 0.00 | H |
| ATOM | 6161 | 1HD2 | LEU | A | 560 | 8.601 | 76.638 | −10.011 | 1.00 | 0.00 | H |
| ATOM | 6162 | 2HD2 | LEU | A | 560 | 9.583 | 77.773 | −10.969 | 1.00 | 0.00 | H |
| ATOM | 6163 | 3HD2 | LEU | A | 560 | 10.228 | 77.096 | −9.455 | 1.00 | 0.00 | H |
| ATOM | 6164 | N | VAL | A | 561 | 11.602 | 81.568 | −6.984 | 1.00 | 0.00 | N |
| ATOM | 6165 | CA | VAL | A | 561 | 12.263 | 82.842 | −6.768 | 1.00 | 0.00 | C |
| ATOM | 6166 | C | VAL | A | 561 | 13.740 | 82.594 | −6.457 | 1.00 | 0.00 | C |
| ATOM | 6167 | O | VAL | A | 561 | 14.571 | 83.465 | −6.709 | 1.00 | 0.00 | O |
| ATOM | 6168 | CB | VAL | A | 561 | 11.542 | 83.644 | −5.666 | 1.00 | 0.00 | C |
| ATOM | 6169 | CG1 | VAL | A | 561 | 12.158 | 85.038 | −5.481 | 1.00 | 0.00 | C |
| ATOM | 6170 | CG2 | VAL | A | 561 | 10.054 | 83.819 | −6.005 | 1.00 | 0.00 | C |
| ATOM | 6171 | H | VAL | A | 561 | 10.990 | 81.242 | −6.249 | 1.00 | 0.00 | H |
| ATOM | 6172 | HA | VAL | A | 561 | 12.102 | 83.486 | −7.632 | 1.00 | 0.00 | H |
| ATOM | 6173 | HB | VAL | A | 561 | 11.606 | 83.104 | −4.721 | 1.00 | 0.00 | H |
| ATOM | 6174 | 1HG1 | VAL | A | 561 | 11.622 | 85.572 | −4.696 | 1.00 | 0.00 | H |
| ATOM | 6175 | 2HG1 | VAL | A | 561 | 13.206 | 84.939 | −5.202 | 1.00 | 0.00 | H |
| ATOM | 6176 | 3HG1 | VAL | A | 561 | 12.082 | 85.596 | −6.415 | 1.00 | 0.00 | H |
| ATOM | 6177 | 1HG2 | VAL | A | 561 | 9.565 | 84.388 | −5.214 | 1.00 | 0.00 | H |
| ATOM | 6178 | 2HG2 | VAL | A | 561 | 9.956 | 84.354 | −6.950 | 1.00 | 0.00 | H |
| ATOM | 6179 | 3HG2 | VAL | A | 561 | 9.583 | 82.840 | −6.092 | 1.00 | 0.00 | H |
| ATOM | 6180 | N | GLU | A | 562 | 14.076 | 81.402 | −5.949 | 1.00 | 0.00 | N |
| ATOM | 6181 | CA | GLU | A | 562 | 15.442 | 80.930 | −6.033 | 1.00 | 0.00 | C |
| ATOM | 6182 | C | GLU | A | 562 | 15.606 | 80.412 | −7.457 | 1.00 | 0.00 | C |
| ATOM | 6183 | O | GLU | A | 562 | 16.422 | 80.936 | −8.207 | 1.00 | 0.00 | O |
| ATOM | 6184 | CB | GLU | A | 562 | 15.763 | 79.851 | −4.980 | 1.00 | 0.00 | C |
| ATOM | 6185 | CG | GLU | A | 562 | 17.276 | 79.724 | −4.697 | 1.00 | 0.00 | C |
| ATOM | 6186 | CD | GLU | A | 562 | 18.116 | 79.457 | −5.938 | 1.00 | 0.00 | C |
| ATOM | 6187 | OE1 | GLU | A | 562 | 17.856 | 78.457 | −6.641 | 1.00 | 0.00 | O |
| ATOM | 6188 | OE2 | GLU | A | 562 | 18.984 | 80.292 | −6.276 | 1.00 | 0.00 | O1− |
| ATOM | 6189 | H | GLU | A | 562 | 13.375 | 80.827 | −5.504 | 1.00 | 0.00 | H |
| ATOM | 6190 | HA | GLU | A | 562 | 16.124 | 81.742 | −5.780 | 1.00 | 0.00 | H |
| ATOM | 6191 | 1HB | GLU | A | 562 | 15.267 | 80.099 | −4.042 | 1.00 | 0.00 | H |
| ATOM | 6192 | 2HB | GLU | A | 562 | 15.408 | 78.882 | −5.333 | 1.00 | 0.00 | H |
| ATOM | 6193 | 1HG | GLU | A | 562 | 17.642 | 80.650 | −4.254 | 1.00 | 0.00 | H |
| ATOM | 6194 | 2HG | GLU | A | 562 | 17.449 | 78.898 | −4.007 | 1.00 | 0.00 | H |
| ATOM | 6195 | N | ALA | A | 563 | 14.864 | 79.364 | −7.821 | 1.00 | 0.00 | N |
| ATOM | 6196 | CA | ALA | A | 563 | 15.291 | 78.356 | −8.784 | 1.00 | 0.00 | C |
| ATOM | 6197 | C | ALA | A | 563 | 15.539 | 78.861 | −10.213 | 1.00 | 0.00 | C |
| ATOM | 6198 | O | ALA | A | 563 | 16.009 | 78.083 | −11.040 | 1.00 | 0.00 | O |
| ATOM | 6199 | CB | ALA | A | 563 | 14.315 | 77.177 | −8.765 | 1.00 | 0.00 | C |
| ATOM | 6200 | H | ALA | A | 563 | 13.945 | 79.233 | −7.422 | 1.00 | 0.00 | H |
| ATOM | 6201 | HA | ALA | A | 563 | 16.156 | 77.821 | −8.391 | 1.00 | 0.00 | H |
| ATOM | 6202 | 1HB | ALA | A | 563 | 14.637 | 76.426 | −9.486 | 1.00 | 0.00 | H |
| ATOM | 6203 | 2HB | ALA | A | 563 | 14.294 | 76.737 | −7.768 | 1.00 | 0.00 | H |
| ATOM | 6204 | 3HB | ALA | A | 563 | 13.316 | 77.527 | −9.028 | 1.00 | 0.00 | H |
| ATOM | 6205 | N | LYS | A | 564 | 15.239 | 80.126 | −10.521 | 1.00 | 0.00 | N |
| ATOM | 6206 | CA | LYS | A | 564 | 15.471 | 80.764 | −11.814 | 1.00 | 0.00 | C |
| ATOM | 6207 | C | LYS | A | 564 | 14.652 | 80.046 | −12.894 | 1.00 | 0.00 | C |
| ATOM | 6208 | O | LYS | A | 564 | 15.190 | 79.216 | −13.623 | 1.00 | 0.00 | O |
| ATOM | 6209 | CB | LYS | A | 564 | 16.977 | 80.770 | −12.167 | 1.00 | 0.00 | C |
| ATOM | 6210 | CG | LYS | A | 564 | 17.860 | 81.539 | −11.165 | 1.00 | 0.00 | C |
| ATOM | 6211 | CD | LYS | A | 564 | 19.238 | 80.886 | −10.921 | 1.00 | 0.00 | C |
| ATOM | 6212 | CE | LYS | A | 564 | 19.205 | 79.558 | −10.135 | 1.00 | 0.00 | C |
| ATOM | 6213 | NZ | LYS | A | 564 | 18.668 | 79.717 | −8.771 | 1.00 | 0.00 | N1+ |
| ATOM | 6214 | H | LYS | A | 564 | 14.815 | 80.727 | −9.829 | 1.00 | 0.00 | H |
| ATOM | 6215 | HA | LYS | A | 564 | 15.219 | 81.822 | −11.748 | 1.00 | 0.00 | H |
| ATOM | 6216 | 1HB | LYS | A | 564 | 17.348 | 79.745 | −12.197 | 1.00 | 0.00 | H |
| ATOM | 6217 | 2HB | LYS | A | 564 | 17.121 | 81.236 | −13.142 | 1.00 | 0.00 | H |
| ATOM | 6218 | 1HG | LYS | A | 564 | 18.040 | 82.547 | −11.538 | 1.00 | 0.00 | H |
| ATOM | 6219 | 2HG | LYS | A | 564 | 17.353 | 81.594 | −10.201 | 1.00 | 0.00 | H |
| ATOM | 6220 | 1HD | LYS | A | 564 | 19.712 | 80.669 | −11.878 | 1.00 | 0.00 | H |
| ATOM | 6221 | 2HD | LYS | A | 564 | 19.868 | 81.569 | −10.351 | 1.00 | 0.00 | H |
| ATOM | 6222 | 1HE | LYS | A | 564 | 18.574 | 78.841 | −10.660 | 1.00 | 0.00 | H |
| ATOM | 6223 | 2HE | LYS | A | 564 | 20.216 | 79.160 | −10.051 | 1.00 | 0.00 | H |
| ATOM | 6224 | 1HZ | LYS | A | 564 | 18.669 | 78.822 | −8.303 | 1.00 | 0.00 | H |
| ATOM | 6225 | 2HZ | LYS | A | 564 | 19.245 | 80.366 | −8.256 | 1.00 | 0.00 | H |
| ATOM | 6226 | 3HZ | LYS | A | 564 | 17.724 | 80.070 | −8.820 | 1.00 | 0.00 | H |
| ATOM | 6227 | N | GLN | A | 565 | 13.361 | 80.361 | −13.005 | 1.00 | 0.00 | N |
| ATOM | 6228 | CA | GLN | A | 565 | 12.396 | 79.505 | −13.684 | 1.00 | 0.00 | C |
| ATOM | 6229 | C | GLN | A | 565 | 11.388 | 80.320 | −14.491 | 1.00 | 0.00 | C |
| ATOM | 6230 | O | GLN | A | 565 | 11.268 | 81.532 | −14.323 | 1.00 | 0.00 | O |
| ATOM | 6231 | CB | GLN | A | 565 | 11.624 | 78.704 | −12.627 | 1.00 | 0.00 | C |
| ATOM | 6232 | CG | GLN | A | 565 | 12.522 | 77.753 | −11.834 | 1.00 | 0.00 | C |
| ATOM | 6233 | CD | GLN | A | 565 | 13.087 | 76.666 | −12.733 | 1.00 | 0.00 | C |
| ATOM | 6234 | NE2 | GLN | A | 565 | 14.401 | 76.536 | −12.836 | 1.00 | 0.00 | N |
| ATOM | 6235 | OE1 | GLN | A | 565 | 12.324 | 75.969 | −13.390 | 1.00 | 0.00 | O |
| ATOM | 6236 | H | GLN | A | 565 | 13.015 | 81.224 | −12.609 | 1.00 | 0.00 | H |
| ATOM | 6237 | HA | GLN | A | 565 | 12.919 | 78.848 | −14.379 | 1.00 | 0.00 | H |
| ATOM | 6238 | 1HB | GLN | A | 565 | 11.159 | 79.390 | −11.919 | 1.00 | 0.00 | H |
| ATOM | 6239 | 2HB | GLN | A | 565 | 10.853 | 78.107 | −13.114 | 1.00 | 0.00 | H |
| ATOM | 6240 | 1HG | GLN | A | 565 | 13.350 | 78.312 | −11.399 | 1.00 | 0.00 | H |
| ATOM | 6241 | 2HG | GLN | A | 565 | 11.943 | 77.284 | −11.039 | 1.00 | 0.00 | H |
| ATOM | 6242 | 1HE2 | GLN | A | 565 | 14.793 | 75.819 | −13.429 | 1.00 | 0.00 | H |
| ATOM | 6243 | 2HE2 | GLN | A | 565 | 15.011 | 77.155 | −12.321 | 1.00 | 0.00 | H |
| ATOM | 6244 | N | GLU | A | 566 | 10.633 | 79.610 | −15.329 | 1.00 | 0.00 | N |
| ATOM | 6245 | CA | GLU | A | 566 | 9.396 | 80.090 | −15.910 | 1.00 | 0.00 | C |
| ATOM | 6246 | C | GLU | A | 566 | 8.364 | 80.271 | −14.786 | 1.00 | 0.00 | C |
| ATOM | 6247 | O | GLU | A | 566 | 8.512 | 79.695 | −13.707 | 1.00 | 0.00 | O |
| ATOM | 6248 | CB | GLU | A | 566 | 8.920 | 79.056 | −16.939 | 1.00 | 0.00 | C |
| ATOM | 6249 | CG | GLU | A | 566 | 9.970 | 78.771 | −18.028 | 1.00 | 0.00 | C |
| ATOM | 6250 | CD | GLU | A | 566 | 9.524 | 77.609 | −18.888 | 1.00 | 0.00 | C |
| ATOM | 6251 | OE1 | GLU | A | 566 | 9.498 | 76.470 | −18.375 | 1.00 | 0.00 | O |
| ATOM | 6252 | OE2 | GLU | A | 566 | 9.095 | 77.827 | −20.042 | 1.00 | 0.00 | O1− |
| ATOM | 6253 | H | GLU | A | 566 | 10.914 | 78.677 | −15.594 | 1.00 | 0.00 | H |
| ATOM | 6254 | HA | GLU | A | 566 | 9.577 | 81.037 | −16.419 | 1.00 | 0.00 | H |
| ATOM | 6255 | 1HB | GLU | A | 566 | 8.699 | 78.116 | −16.435 | 1.00 | 0.00 | H |
| ATOM | 6256 | 2HB | GLU | A | 566 | 8.020 | 79.423 | −17.433 | 1.00 | 0.00 | H |
| ATOM | 6257 | 1HG | GLU | A | 566 | 10.093 | 79.653 | −18.655 | 1.00 | 0.00 | H |
| ATOM | 6258 | 2HG | GLU | A | 566 | 10.922 | 78.523 | −17.559 | 1.00 | 0.00 | H |
| ATOM | 6259 | N | VAL | A | 567 | 7.311 | 81.048 | −15.041 | 1.00 | 0.00 | N |
| ATOM | 6260 | CA | VAL | A | 567 | 6.393 | 81.519 | −14.014 | 1.00 | 0.00 | C |
| ATOM | 6261 | C | VAL | A | 567 | 5.008 | 80.902 | −14.258 | 1.00 | 0.00 | C |
| ATOM | 6262 | O | VAL | A | 567 | 4.434 | 81.090 | −15.329 | 1.00 | 0.00 | O |
| ATOM | 6263 | CB | VAL | A | 567 | 6.327 | 83.057 | −14.061 | 1.00 | 0.00 | C |
| ATOM | 6264 | CG1 | VAL | A | 567 | 5.444 | 83.599 | −12.930 | 1.00 | 0.00 | C |
| ATOM | 6265 | CG2 | VAL | A | 567 | 7.726 | 83.677 | −13.924 | 1.00 | 0.00 | C |
| ATOM | 6266 | H | VAL | A | 567 | 7.117 | 81.340 | −15.989 | 1.00 | 0.00 | H |
| ATOM | 6267 | HA | VAL | A | 567 | 6.773 | 81.240 | −13.031 | 1.00 | 0.00 | H |
| ATOM | 6268 | HB | VAL | A | 567 | 5.919 | 83.374 | −15.021 | 1.00 | 0.00 | H |
| ATOM | 6269 | 1HG1 | VAL | A | 567 | 5.412 | 84.688 | −12.984 | 1.00 | 0.00 | H |
| ATOM | 6270 | 2HG1 | VAL | A | 567 | 4.434 | 83.202 | −13.035 | 1.00 | 0.00 | H |
| ATOM | 6271 | 3HG1 | VAL | A | 567 | 5.856 | 83.295 | −11.969 | 1.00 | 0.00 | H |
| ATOM | 6272 | 1HG2 | VAL | A | 567 | 7.648 | 84.764 | −13.960 | 1.00 | 0.00 | H |
| ATOM | 6273 | 2HG2 | VAL | A | 567 | 8.165 | 83.377 | −12.972 | 1.00 | 0.00 | H |
| ATOM | 6274 | 3HG2 | VAL | A | 567 | 8.360 | 83.331 | −14.740 | 1.00 | 0.00 | H |
| ATOM | 6275 | N | PRO | A | 568 | 4.440 | 80.187 | −13.277 | 1.00 | 0.00 | N |
| ATOM | 6276 | CA | PRO | A | 568 | 3.067 | 79.711 | −13.337 | 1.00 | 0.00 | C |
| ATOM | 6277 | C | PRO | A | 568 | 2.072 | 80.863 | −13.131 | 1.00 | 0.00 | C |
| ATOM | 6278 | O | PRO | A | 568 | 1.406 | 80.939 | −12.097 | 1.00 | 0.00 | O |
| ATOM | 6279 | CB | PRO | A | 568 | 2.965 | 78.650 | −12.233 | 1.00 | 0.00 | C |
| ATOM | 6280 | CG | PRO | A | 568 | 4.390 | 78.118 | −12.122 | 1.00 | 0.00 | C |
| ATOM | 6281 | CD | PRO | A | 568 | 5.205 | 79.392 | −12.332 | 1.00 | 0.00 | C |
| ATOM | 6282 | HA | PRO | A | 568 | 2.889 | 79.234 | −14.301 | 1.00 | 0.00 | H |
| ATOM | 6283 | 1HB | PRO | A | 568 | 2.622 | 79.117 | −11.310 | 1.00 | 0.00 | H |
| ATOM | 6284 | 2HB | PRO | A | 568 | 2.256 | 77.879 | −12.534 | 1.00 | 0.00 | H |
| ATOM | 6285 | 1HG | PRO | A | 568 | 4.535 | 77.670 | −11.139 | 1.00 | 0.00 | H |
| ATOM | 6286 | 2HG | PRO | A | 568 | 4.559 | 77.366 | −12.892 | 1.00 | 0.00 | H |
| ATOM | 6287 | 1HD | PRO | A | 568 | 6.187 | 79.173 | −12.751 | 1.00 | 0.00 | H |
| ATOM | 6288 | 2HD | PRO | A | 568 | 5.324 | 79.948 | −11.401 | 1.00 | 0.00 | H |
| ATOM | 6289 | N | SER | A | 569 | 1.983 | 81.771 | −14.102 | 1.00 | 0.00 | N |
| ATOM | 6290 | CA | SER | A | 569 | 1.220 | 82.996 | −13.953 | 1.00 | 0.00 | C |
| ATOM | 6291 | C | SER | A | 569 | −0.274 | 82.724 | −13.756 | 1.00 | 0.00 | C |
| ATOM | 6292 | O | SER | A | 569 | −1.025 | 82.592 | −14.720 | 1.00 | 0.00 | O |
| ATOM | 6293 | CB | SER | A | 569 | 1.492 | 83.920 | −15.140 | 1.00 | 0.00 | C |
| ATOM | 6294 | OG | SER | A | 569 | 2.876 | 84.216 | −15.195 | 1.00 | 0.00 | O |
| ATOM | 6295 | H | SER | A | 569 | 2.457 | 81.619 | −14.980 | 1.00 | 0.00 | H |
| ATOM | 6296 | HA | SER | A | 569 | 1.645 | 83.593 | −13.146 | 1.00 | 0.00 | H |
| ATOM | 6297 | 1HB | SER | A | 569 | 1.188 | 83.426 | −16.063 | 1.00 | 0.00 | H |
| ATOM | 6298 | 2HB | SER | A | 569 | 0.926 | 84.844 | −15.019 | 1.00 | 0.00 | H |
| ATOM | 6299 | HG | SER | A | 569 | 3.050 | 84.797 | −15.940 | 1.00 | 0.00 | H |
| ATOM | 6300 | N | TRP | A | 570 | −0.678 | 82.674 | −12.486 | 1.00 | 0.00 | N |
| ATOM | 6301 | CA | TRP | A | 570 | −1.984 | 82.399 | −11.910 | 1.00 | 0.00 | C |
| ATOM | 6302 | C | TRP | A | 570 | −1.711 | 82.073 | −10.437 | 1.00 | 0.00 | C |
| ATOM | 6303 | O | TRP | A | 570 | −2.362 | 82.579 | −9.521 | 1.00 | 0.00 | O |
| ATOM | 6304 | CB | TRP | A | 570 | −2.727 | 81.241 | −12.599 | 1.00 | 0.00 | C |
| ATOM | 6305 | CG | TRP | A | 570 | −2.107 | 79.879 | −12.487 | 1.00 | 0.00 | C |
| ATOM | 6306 | CD1 | TRP | A | 570 | −1.190 | 79.346 | −13.325 | 1.00 | 0.00 | C |
| ATOM | 6307 | CD2 | TRP | A | 570 | −2.343 | 78.865 | −11.465 | 1.00 | 0.00 | C |
| ATOM | 6308 | CE2 | TRP | A | 570 | −1.532 | 77.729 | −11.757 | 1.00 | 0.00 | C |
| ATOM | 6309 | CE3 | TRP | A | 570 | −3.153 | 78.786 | −10.309 | 1.00 | 0.00 | C |
| ATOM | 6310 | NE1 | TRP | A | 570 | −0.839 | 78.079 | −12.898 | 1.00 | 0.00 | N |
| ATOM | 6311 | CZ2 | TRP | A | 570 | −1.550 | 76.581 | −10.951 | 1.00 | 0.00 | C |
| ATOM | 6312 | CZ3 | TRP | A | 570 | −3.162 | 77.644 | −9.488 | 1.00 | 0.00 | C |
| ATOM | 6313 | CH2 | TRP | A | 570 | −2.364 | 76.537 | −9.810 | 1.00 | 0.00 | C |
| ATOM | 6314 | H | TRP | A | 570 | −0.020 | 82.847 | −11.740 | 1.00 | 0.00 | H |
| ATOM | 6315 | HA | TRP | A | 570 | −2.628 | 83.270 | −12.035 | 1.00 | 0.00 | H |
| ATOM | 6316 | 1HB | TRP | A | 570 | −3.726 | 81.145 | −12.173 | 1.00 | 0.00 | H |
| ATOM | 6317 | 2HB | TRP | A | 570 | −2.806 | 81.442 | −13.667 | 1.00 | 0.00 | H |
| ATOM | 6318 | HD1 | TRP | A | 570 | −0.846 | 79.912 | −14.179 | 1.00 | 0.00 | H |
| ATOM | 6319 | HE3 | TRP | A | 570 | −3.787 | 79.617 | −10.036 | 1.00 | 0.00 | H |
| ATOM | 6320 | HE1 | TRP | A | 570 | −0.154 | 77.545 | −13.413 | 1.00 | 0.00 | H |
| ATOM | 6321 | HZ2 | TRP | A | 570 | −0.936 | 75.730 | −11.209 | 1.00 | 0.00 | H |
| ATOM | 6322 | HZ3 | TRP | A | 570 | −3.785 | 77.618 | −8.606 | 1.00 | 0.00 | H |
| ATOM | 6323 | HH2 | TRP | A | 570 | −2.374 | 75.654 | −9.187 | 1.00 | 0.00 | H |
| ATOM | 6324 | N | LEU | A | 571 | −0.684 | 81.257 | −10.198 | 1.00 | 0.00 | N |
| ATOM | 6325 | CA | LEU | A | 571 | −0.310 | 80.826 | −8.865 | 1.00 | 0.00 | C |
| ATOM | 6326 | C | LEU | A | 571 | 0.301 | 82.010 | −8.113 | 1.00 | 0.00 | C |
| ATOM | 6327 | O | LEU | A | 571 | 0.222 | 82.081 | −6.892 | 1.00 | 0.00 | O |
| ATOM | 6328 | CB | LEU | A | 571 | 0.648 | 79.635 | −8.972 | 1.00 | 0.00 | C |
| ATOM | 6329 | CG | LEU | A | 571 | 1.046 | 79.027 | −7.618 | 1.00 | 0.00 | C |
| ATOM | 6330 | CD1 | LEU | A | 571 | −0.145 | 78.402 | −6.882 | 1.00 | 0.00 | C |
| ATOM | 6331 | CD2 | LEU | A | 571 | 2.112 | 77.951 | −7.848 | 1.00 | 0.00 | C |
| ATOM | 6332 | H | LEU | A | 571 | −0.126 | 80.911 | −10.965 | 1.00 | 0.00 | H |
| ATOM | 6333 | HA | LEU | A | 571 | −1.183 | 80.416 | −8.357 | 1.00 | 0.00 | H |
| ATOM | 6334 | 1HB | LEU | A | 571 | 0.179 | 78.842 | −9.554 | 1.00 | 0.00 | H |
| ATOM | 6335 | 2HB | LEU | A | 571 | 1.568 | 79.951 | −9.464 | 1.00 | 0.00 | H |
| ATOM | 6336 | HG | LEU | A | 571 | 1.482 | 79.800 | −6.985 | 1.00 | 0.00 | H |
| ATOM | 6337 | 1HD1 | LEU | A | 571 | 0.191 | 77.987 | −5.932 | 1.00 | 0.00 | H |
| ATOM | 6338 | 2HD1 | LEU | A | 571 | −0.901 | 79.166 | −6.698 | 1.00 | 0.00 | H |
| ATOM | 6339 | 3HD1 | LEU | A | 571 | −0.574 | 77.608 | −7.493 | 1.00 | 0.00 | H |
| ATOM | 6340 | 1HD2 | LEU | A | 571 | 2.400 | 77.514 | −6.892 | 1.00 | 0.00 | H |
| ATOM | 6341 | 2HD2 | LEU | A | 571 | 1.709 | 77.173 | −8.496 | 1.00 | 0.00 | H |
| ATOM | 6342 | 3HD2 | LEU | A | 571 | 2.986 | 78.400 | −8.320 | 1.00 | 0.00 | H |
| ATOM | 6343 | N | GLU | A | 572 | 0.852 | 82.980 | −8.844 | 1.00 | 0.00 | N |
| ATOM | 6344 | CA | GLU | A | 572 | 1.341 | 84.233 | −8.285 | 1.00 | 0.00 | C |
| ATOM | 6345 | C | GLU | A | 572 | 0.182 | 85.156 | −7.876 | 1.00 | 0.00 | C |
| ATOM | 6346 | O | GLU | A | 572 | 0.393 | 86.314 | −7.523 | 1.00 | 0.00 | O |
| ATOM | 6347 | CB | GLU | A | 572 | 2.340 | 84.906 | −9.241 | 1.00 | 0.00 | C |
| ATOM | 6348 | CG | GLU | A | 572 | 2.060 | 84.678 | −10.734 | 1.00 | 0.00 | C |
| ATOM | 6349 | CD | GLU | A | 572 | 2.971 | 85.526 | −11.614 | 1.00 | 0.00 | C |
| ATOM | 6350 | OE1 | GLU | A | 572 | 3.935 | 86.094 | −11.064 | 1.00 | 0.00 | O |
| ATOM | 6351 | OE2 | GLU | A | 572 | 2.695 | 85.568 | −12.832 | 1.00 | 0.00 | O1− |
| ATOM | 6352 | H | GLU | A | 572 | 0.949 | 82.867 | −9.843 | 1.00 | 0.00 | H |
| ATOM | 6353 | HA | GLU | A | 572 | 2.044 | 84.023 | −7.479 | 1.00 | 0.00 | H |
| ATOM | 6354 | 1HB | GLU | A | 572 | 2.328 | 85.984 | −9.082 | 1.00 | 0.00 | H |
| ATOM | 6355 | 2HB | GLU | A | 572 | 3.342 | 84.522 | −9.049 | 1.00 | 0.00 | H |
| ATOM | 6356 | 1HG | GLU | A | 572 | 2.226 | 83.629 | −10.980 | 1.00 | 0.00 | H |
| ATOM | 6357 | 2HG | GLU | A | 572 | 1.026 | 84.944 | −10.955 | 1.00 | 0.00 | H |
| ATOM | 6358 | N | ASN | A | 573 | −1.058 | 84.665 | −7.907 | 1.00 | 0.00 | N |
| ATOM | 6359 | CA | ASN | A | 573 | −2.196 | 85.306 | −7.268 | 1.00 | 0.00 | C |
| ATOM | 6360 | C | ASN | A | 573 | −2.767 | 84.332 | −6.245 | 1.00 | 0.00 | C |
| ATOM | 6361 | O | ASN | A | 573 | −3.040 | 84.729 | −5.117 | 1.00 | 0.00 | O |
| ATOM | 6362 | CB | ASN | A | 573 | −3.264 | 85.719 | −8.290 | 1.00 | 0.00 | C |
| ATOM | 6363 | CG | ASN | A | 573 | −2.961 | 87.009 | −9.052 | 1.00 | 0.00 | C |
| ATOM | 6364 | ND2 | ASN | A | 573 | −1.744 | 87.543 | −8.999 | 1.00 | 0.00 | N |
| ATOM | 6365 | OD1 | ASN | A | 573 | −3.840 | 87.549 | −9.716 | 1.00 | 0.00 | O |
| ATOM | 6366 | H | ASN | A | 573 | −1.247 | 83.801 | −8.394 | 1.00 | 0.00 | H |
| ATOM | 6367 | HA | ASN | A | 573 | −1.877 | 86.242 | −6.810 | 1.00 | 0.00 | H |
| ATOM | 6368 | 1HB | ASN | A | 573 | −3.376 | 84.933 | −9.037 | 1.00 | 0.00 | H |
| ATOM | 6369 | 2HB | ASN | A | 573 | −4.215 | 85.873 | −7.780 | 1.00 | 0.00 | H |
| ATOM | 6370 | 1HD2 | ASN | A | 573 | −1.545 | 88.396 | −9.503 | 1.00 | 0.00 | H |
| ATOM | 6371 | 2HD2 | ASN | A | 573 | −1.020 | 87.096 | −8.455 | 1.00 | 0.00 | H |
| ATOM | 6372 | N | MET | A | 574 | −2.938 | 83.056 | −6.604 | 1.00 | 0.00 | N |
| ATOM | 6373 | CA | MET | A | 574 | −3.536 | 82.100 | −5.674 | 1.00 | 0.00 | C |
| ATOM | 6374 | C | MET | A | 574 | −2.607 | 81.775 | −4.499 | 1.00 | 0.00 | C |
| ATOM | 6375 | O | MET | A | 574 | −3.063 | 81.293 | −3.469 | 1.00 | 0.00 | O |
| ATOM | 6376 | CB | MET | A | 574 | −4.021 | 80.838 | −6.395 | 1.00 | 0.00 | C |
| ATOM | 6377 | CG | MET | A | 574 | −5.100 | 81.186 | −7.429 | 1.00 | 0.00 | C |
| ATOM | 6378 | SD | MET | A | 574 | −6.083 | 79.784 | −8.027 | 1.00 | 0.00 | S |
| ATOM | 6379 | CE | MET | A | 574 | −7.156 | 79.513 | −6.589 | 1.00 | 0.00 | C |
| ATOM | 6380 | H | MET | A | 574 | −2.653 | 82.747 | −7.523 | 1.00 | 0.00 | H |
| ATOM | 6381 | HA | MET | A | 574 | −4.518 | 82.459 | −5.365 | 1.00 | 0.00 | H |
| ATOM | 6382 | 1HB | MET | A | 574 | −3.182 | 80.367 | −6.907 | 1.00 | 0.00 | H |
| ATOM | 6383 | 2HB | MET | A | 574 | −4.441 | 80.142 | −5.669 | 1.00 | 0.00 | H |
| ATOM | 6384 | 1HG | MET | A | 574 | −5.805 | 81.895 | −6.995 | 1.00 | 0.00 | H |
| ATOM | 6385 | 2HG | MET | A | 574 | −4.633 | 81.632 | −8.307 | 1.00 | 0.00 | H |
| ATOM | 6386 | 1HE | MET | A | 574 | −7.829 | 78.680 | −6.789 | 1.00 | 0.00 | H |
| ATOM | 6387 | 2HE | MET | A | 574 | −6.544 | 79.284 | −5.716 | 1.00 | 0.00 | H |
| ATOM | 6388 | 3HE | MET | A | 574 | −7.740 | 80.413 | −6.396 | 1.00 | 0.00 | H |
| ATOM | 6389 | N | ALA | A | 575 | −1.315 | 82.091 | −4.595 | 1.00 | 0.00 | N |
| ATOM | 6390 | CA | ALA | A | 575 | −0.416 | 82.022 | −3.447 | 1.00 | 0.00 | C |
| ATOM | 6391 | C | ALA | A | 575 | −0.664 | 83.189 | −2.483 | 1.00 | 0.00 | C |
| ATOM | 6392 | O | ALA | A | 575 | 0.094 | 83.389 | −1.534 | 1.00 | 0.00 | O |
| ATOM | 6393 | CB | ALA | A | 575 | 1.038 | 81.992 | −3.924 | 1.00 | 0.00 | C |
| ATOM | 6394 | H | ALA | A | 575 | −0.932 | 82.389 | −5.481 | 1.00 | 0.00 | H |
| ATOM | 6395 | HA | ALA | A | 575 | −0.534 | 81.059 | −2.950 | 1.00 | 0.00 | H |
| ATOM | 6396 | 1HB | ALA | A | 575 | 1.703 | 81.940 | −3.062 | 1.00 | 0.00 | H |
| ATOM | 6397 | 2HB | ALA | A | 575 | 1.196 | 81.119 | −4.557 | 1.00 | 0.00 | H |
| ATOM | 6398 | 3HB | ALA | A | 575 | 1.252 | 82.896 | −4.493 | 1.00 | 0.00 | H |
| ATOM | 6399 | N | TYR | A | 576 | −1.701 | 83.985 | −2.747 | 1.00 | 0.00 | N |
| ATOM | 6400 | CA | TYR | A | 576 | −2.199 | 85.064 | −1.919 | 1.00 | 0.00 | C |
| ATOM | 6401 | C | TYR | A | 576 | −3.732 | 85.020 | −1.989 | 1.00 | 0.00 | C |
| ATOM | 6402 | O | TYR | A | 576 | −4.396 | 86.058 | −1.981 | 1.00 | 0.00 | O |
| ATOM | 6403 | CB | TYR | A | 576 | −1.628 | 86.397 | −2.427 | 1.00 | 0.00 | C |
| ATOM | 6404 | CG | TYR | A | 576 | −0.124 | 86.376 | −2.638 | 1.00 | 0.00 | C |
| ATOM | 6405 | CD1 | TYR | A | 576 | 0.757 | 86.560 | −1.557 | 1.00 | 0.00 | C |
| ATOM | 6406 | CD2 | TYR | A | 576 | 0.416 | 86.131 | −3.915 | 1.00 | 0.00 | C |
| ATOM | 6407 | CE1 | TYR | A | 576 | 2.147 | 86.477 | −1.745 | 1.00 | 0.00 | C |
| ATOM | 6408 | CE2 | TYR | A | 576 | 1.806 | 86.048 | −4.105 | 1.00 | 0.00 | C |
| ATOM | 6409 | CZ | TYR | A | 576 | 2.686 | 86.210 | −3.018 | 1.00 | 0.00 | C |
| ATOM | 6410 | OH | TYR | A | 576 | 4.037 | 86.113 | −3.190 | 1.00 | 0.00 | O |
| ATOM | 6411 | H | TYR | A | 576 | −2.226 | 83.863 | −3.601 | 1.00 | 0.00 | H |
| ATOM | 6412 | HA | TYR | A | 576 | −1.829 | 84.941 | −0.900 | 1.00 | 0.00 | H |
| ATOM | 6413 | 1HB | TYR | A | 576 | −2.086 | 86.649 | −3.383 | 1.00 | 0.00 | H |
| ATOM | 6414 | 2HB | TYR | A | 576 | −1.842 | 87.184 | −1.704 | 1.00 | 0.00 | H |
| ATOM | 6415 | HD1 | TYR | A | 576 | 0.367 | 86.768 | −0.572 | 1.00 | 0.00 | H |
| ATOM | 6416 | HD2 | TYR | A | 576 | −0.240 | 86.004 | −4.764 | 1.00 | 0.00 | H |
| ATOM | 6417 | HE1 | TYR | A | 576 | 2.815 | 86.618 | −0.907 | 1.00 | 0.00 | H |
| ATOM | 6418 | HE2 | TYR | A | 576 | 2.205 | 85.858 | −5.090 | 1.00 | 0.00 | H |
| ATOM | 6419 | HH | TYR | A | 576 | 4.230 | 85.929 | −4.112 | 1.00 | 0.00 | H |
| ATOM | 6420 | N | GLU | A | 577 | −4.277 | 83.803 | −2.085 | 1.00 | 0.00 | N |
| ATOM | 6421 | CA | GLU | A | 577 | −5.700 | 83.521 | −2.197 | 1.00 | 0.00 | C |
| ATOM | 6422 | C | GLU | A | 577 | −6.494 | 84.232 | −1.089 | 1.00 | 0.00 | C |
| ATOM | 6423 | O | GLU | A | 577 | −6.017 | 84.390 | 0.036 | 1.00 | 0.00 | O |
| ATOM | 6424 | CB | GLU | A | 577 | −5.886 | 81.993 | −2.171 | 1.00 | 0.00 | C |
| ATOM | 6425 | CG | GLU | A | 577 | −7.323 | 81.463 | −2.094 | 1.00 | 0.00 | C |
| ATOM | 6426 | CD | GLU | A | 577 | −8.184 | 81.877 | −3.272 | 1.00 | 0.00 | C |
| ATOM | 6427 | OE1 | GLU | A | 577 | −8.841 | 80.984 | −3.840 | 1.00 | 0.00 | O |
| ATOM | 6428 | OE2 | GLU | A | 577 | −8.304 | 83.098 | −3.488 | 1.00 | 0.00 | O1− |
| ATOM | 6429 | H | GLU | A | 577 | −3.686 | 82.984 | −2.085 | 1.00 | 0.00 | H |
| ATOM | 6430 | HA | GLU | A | 577 | −6.051 | 83.802 | −3.190 | 1.00 | 0.00 | H |
| ATOM | 6431 | 1HB | GLU | A | 577 | −5.465 | 81.560 | −3.078 | 1.00 | 0.00 | H |
| ATOM | 6432 | 2HB | GLU | A | 577 | −5.377 | 81.579 | −1.300 | 1.00 | 0.00 | H |
| ATOM | 6433 | 1HG | GLU | A | 577 | −7.308 | 80.374 | −2.067 | 1.00 | 0.00 | H |
| ATOM | 6434 | 2HG | GLU | A | 577 | −7.802 | 81.842 | −1.191 | 1.00 | 0.00 | H |
| ATOM | 6435 | N | HIS | A | 578 | −7.710 | 84.665 | −1.417 | 1.00 | 0.00 | N |
| ATOM | 6436 | CA | HIS | A | 578 | −8.659 | 85.329 | −0.539 | 1.00 | 0.00 | C |
| ATOM | 6437 | C | HIS | A | 578 | −10.078 | 84.816 | −0.843 | 1.00 | 0.00 | C |
| ATOM | 6438 | O | HIS | A | 578 | −11.063 | 85.502 | −0.560 | 1.00 | 0.00 | O |
| ATOM | 6439 | CB | HIS | A | 578 | −8.569 | 86.853 | −0.742 | 1.00 | 0.00 | C |
| ATOM | 6440 | CG | HIS | A | 578 | −7.452 | 87.542 | 0.004 | 1.00 | 0.00 | C |
| ATOM | 6441 | CD2 | HIS | A | 578 | −7.475 | 88.455 | 1.030 | 1.00 | 0.00 | C |
| ATOM | 6442 | ND1 | HIS | A | 578 | −6.106 | 87.352 | −0.276 | 1.00 | 0.00 | N |
| ATOM | 6443 | CE1 | HIS | A | 578 | −5.390 | 88.110 | 0.568 | 1.00 | 0.00 | C |
| ATOM | 6444 | NE2 | HIS | A | 578 | −6.182 | 88.820 | 1.372 | 1.00 | 0.00 | N |
| ATOM | 6445 | H | HIS | A | 578 | −8.053 | 84.543 | −2.359 | 1.00 | 0.00 | H |
| ATOM | 6446 | HA | HIS | A | 578 | −8.390 | 85.138 | 0.499 | 1.00 | 0.00 | H |
| ATOM | 6447 | 1HB | HIS | A | 578 | −8.414 | 87.072 | −1.798 | 1.00 | 0.00 | H |
| ATOM | 6448 | 2HB | HIS | A | 578 | −9.496 | 87.320 | −0.407 | 1.00 | 0.00 | H |
| ATOM | 6449 | HD2 | HIS | A | 578 | −8.418 | 88.779 | 1.444 | 1.00 | 0.00 | H |
| ATOM | 6450 | HD1 | HIS | A | 578 | −5.828 | 86.722 | −1.016 | 1.00 | 0.00 | H |
| ATOM | 6451 | HE1 | HIS | A | 578 | −4.311 | 88.087 | 0.532 | 1.00 | 0.00 | H |
| ATOM | 6452 | N | HIS | A | 579 | −10.211 | 83.640 | −1.458 | 1.00 | 0.00 | N |
| ATOM | 6453 | CA | HIS | A | 579 | −11.482 | 83.026 | −1.836 | 1.00 | 0.00 | C |
| ATOM | 6454 | C | HIS | A | 579 | −11.547 | 81.576 | −1.348 | 1.00 | 0.00 | C |
| ATOM | 6455 | O | HIS | A | 579 | −12.307 | 81.282 | −0.426 | 1.00 | 0.00 | O |
| ATOM | 6456 | CB | HIS | A | 579 | −11.732 | 83.130 | −3.351 | 1.00 | 0.00 | C |
| ATOM | 6457 | CG | HIS | A | 579 | −12.230 | 84.473 | −3.835 | 1.00 | 0.00 | C |
| ATOM | 6458 | CD2 | HIS | A | 579 | −12.744 | 84.873 | −5.045 | 1.00 | 0.00 | C |
| ATOM | 6459 | ND1 | HIS | A | 579 | −12.262 | 85.613 | −3.039 | 1.00 | 0.00 | N |
| ATOM | 6460 | CE1 | HIS | A | 579 | −12.770 | 86.620 | −3.766 | 1.00 | 0.00 | C |
| ATOM | 6461 | NE2 | HIS | A | 579 | −13.099 | 86.214 | −4.989 | 1.00 | 0.00 | N |
| ATOM | 6462 | H | HIS | A | 579 | −9.392 | 83.100 | −1.699 | 1.00 | 0.00 | H |
| ATOM | 6463 | HA | HIS | A | 579 | −12.305 | 83.637 | −1.465 | 1.00 | 0.00 | H |
| ATOM | 6464 | 1HB | HIS | A | 579 | −10.803 | 82.934 | −3.887 | 1.00 | 0.00 | H |
| ATOM | 6465 | 2HB | HIS | A | 579 | −12.483 | 82.397 | −3.647 | 1.00 | 0.00 | H |
| ATOM | 6466 | HD2 | HIS | A | 579 | −12.819 | 84.167 | −5.859 | 1.00 | 0.00 | H |
| ATOM | 6467 | HD1 | HIS | A | 579 | −11.931 | 85.573 | −2.085 | 1.00 | 0.00 | H |
| ATOM | 6468 | HE1 | HIS | A | 579 | −12.864 | 87.605 | −3.332 | 1.00 | 0.00 | H |
| ATOM | 6469 | N | TYR | A | 580 | −10.799 | 80.662 | −1.963 | 1.00 | 0.00 | N |
| ATOM | 6470 | CA | TYR | A | 580 | −10.795 | 79.248 | −1.613 | 1.00 | 0.00 | C |
| ATOM | 6471 | C | TYR | A | 580 | −9.428 | 78.633 | −1.905 | 1.00 | 0.00 | C |
| ATOM | 6472 | O | TYR | A | 580 | −8.725 | 78.252 | −0.980 | 1.00 | 0.00 | O |
| ATOM | 6473 | CB | TYR | A | 580 | −11.895 | 78.510 | −2.389 | 1.00 | 0.00 | C |
| ATOM | 6474 | CG | TYR | A | 580 | −12.108 | 77.068 | −1.959 | 1.00 | 0.00 | C |
| ATOM | 6475 | CD1 | TYR | A | 580 | −11.545 | 75.996 | −2.681 | 1.00 | 0.00 | C |
| ATOM | 6476 | CD2 | TYR | A | 580 | −12.883 | 76.779 | −0.820 | 1.00 | 0.00 | C |
| ATOM | 6477 | CE1 | TYR | A | 580 | −11.763 | 74.664 | −2.278 | 1.00 | 0.00 | C |
| ATOM | 6478 | CE2 | TYR | A | 580 | −13.100 | 75.450 | −0.418 | 1.00 | 0.00 | C |
| ATOM | 6479 | CZ | TYR | A | 580 | −12.548 | 74.381 | −1.145 | 1.00 | 0.00 | C |
| ATOM | 6480 | OH | TYR | A | 580 | −12.778 | 73.093 | −0.747 | 1.00 | 0.00 | O |
| ATOM | 6481 | H | TYR | A | 580 | −10.188 | 80.935 | −2.720 | 1.00 | 0.00 | H |
| ATOM | 6482 | HA | TYR | A | 580 | −11.055 | 79.133 | −0.560 | 1.00 | 0.00 | H |
| ATOM | 6483 | HXT | TYR | A | 580 | −9.081 | 78.534 | −2.923 | 1.00 | 0.00 | H |
| ATOM | 6484 | 1HB | TYR | A | 580 | −12.845 | 79.026 | −2.252 | 1.00 | 0.00 | H |
| ATOM | 6485 | 2HB | TYR | A | 580 | −11.642 | 78.491 | −3.449 | 1.00 | 0.00 | H |
| ATOM | 6486 | HD1 | TYR | A | 580 | −10.940 | 76.191 | −3.554 | 1.00 | 0.00 | H |
| ATOM | 6487 | HD2 | TYR | A | 580 | −13.318 | 77.583 | −0.245 | 1.00 | 0.00 | H |
| ATOM | 6488 | HE1 | TYR | A | 580 | −11.327 | 73.851 | −2.840 | 1.00 | 0.00 | H |
| ATOM | 6489 | HE2 | TYR | A | 580 | −13.696 | 75.241 | 0.458 | 1.00 | 0.00 | H |
| ATOM | 6490 | HH | TYR | A | 580 | −13.329 | 73.094 | 0.039 | 1.00 | 0.00 | H |

ENDMDL
END

### Virtual Docking

The virtual docking and scoring of a large number of molecules in a protein active site has proven to be a useful method for selecting molecules for screening. It has the potential to be less biased than pharmacophore-based methods, since the only assumption one must make is the region of the protein surface to target.

Starting from the structure reported in Appendix I, a virtual docking protocol (using Gold 3.0.1) has been applyied to a collection of 220000 compounds from the Asinex database that have been opportunely treated: the 2D structures have been initially converted into the corresponding 3D structures using the Cerius² software and the 3D compounds have been filtered on the basis of the followings criteria:
Molecular weight≦550
Rotable bonds≦10
Number of H-bond acceptors≦11
Number of H-bond donors≦6
AlogP98≦6
Lipinski rule of five violations≦1

The resulting compounds have been submitted to conformational analysis (Cerius default parameters) and finally treated with OpenBabel using the following options: -d (to remove hydroges); -p (add hydrogens at physiological pH); -c (reset coordinates).

The resulting database of commercially available compounds has been automatically docked into the RNA binding site using the GOLD default parameters with the exception of the followings:

Population size=100

### Select_pressure=1.1

### N_island=5

### Max Ops=100000

### Niche_size=2

### Pt_crosswt=95

### Allele_mutatewt=95

### Migratewt=10

### Radius=10

### Origin=6.38; −29.48; 26.06

### Floodfill_atom_no=2465

### No_run=10

Scoring function=chemscore

The 100 best ranking compounds (according to chemscore) were than re-docked into the RNA binding site increasing the number of runs up to 100. Visual inspection allowed to select the best candidate compounds defined by a chemscore value equal to or major than 25.

### EXAMPLES

### Example 1

### One-Pot Two-Steps Microwave-Assisted Synthesis of N-Substituted 5-Alkylidene-thiazol-4-ones

General Procedure: To a solution of bis(carboxymethyl)trithiocarbonate 1 (1 eq.) in DME, TEA (1 eq.) and the amine 2 (R₁NH₂, 1 eq.) were added. The reaction mixture was heated at 90° C. under microwave irradiation for 10 min. After this time, the aldehyde (R₂CHO, 1 eq.) was added and the mixture was heated at 110° C. under microwave irradiation for 5 min. The reaction mixture was evaporated to dryness and the residue was additioned with MeOH; the final rhodanine derivative was obtained as a pure precipitate upon standing, isolated by filtration, washed with hexane and finally dried under high vacuum.

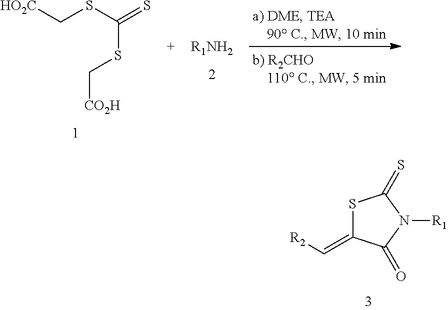

(Z)-5-(3-bromobenzylidene)-3-phenethyl-2-thioxothiazolidin-4-one (3a). Yellow solid. Mp 102-104° C. ¹H NMR (400 MHz, CDCl₃) 7.56-7.49 (3H, m), δ 7.36-7.13 (7H, m), δ 4.30-4.26 (2H, t, J=7.8 Hz), δ 2.96-2.92 (2H, t, J=7.8 Hz) ppm. ¹³C NMR (100 MHz, CDCl₃) 192.5, 167.3, 137.3, 137.1, 135.3, 133.4, 133.1, 130.9, 130.7, 128.9, 126.9, 124.06, 123.0, 45.75, 32.93 ppm. Anal. Calcd for (C₁₈H₁₄BrNOS₂): C, 53.47; H, 3.49; N, 3.46. Found: C, 53.36; H, 3.40; N, 3.55.

(Z)-5-(3-bromobenzylidene)-3-benzyl-2-thioxothiazolidin-4-one (3b). Yellow solid. Mp 112-113° C. ¹H NMR (400 MHz, CDCl₃) δ 7.55-7.47 (3H, m), δ 7.41-7.24 (7H, m), δ 5.25 (2H, s) ppm. ¹³C NMR (100 MHz, CDCl₃) 192.5, 167.6, 135.3, 134.7, 133.5, 133.2, 131.2, 130.8, 129.0, 128.8, 128.6, 128.3, 124.7, 123.5, 47.6 ppm. Anal. Calcd for (C₁₇H₁₂BrNOS₂): C, 52.31; H, 3.10; N, 3.59. Found: C, 53.30; H, 3.21; N, 3.48.

(Z)-5-(3-bromobenzylidene)-3-ethynyl-2-thioxothiazolidin-4-one (3c). Orange solid. Mp 159-161° C. ¹H NMR (400 MHz, CDCl₃) δ 7.60 (1H, s), δ 7.55 (1H,$), δ 7.51-7.49 (1H, d, J=7.7 Hz), δ 7.35-7.34 (1H, d, J=7.7 Hz), δ 7.29-7.19 (1H, t, J=7.7 Hz), δ 4.81-4.80 (2H, d, J=2.1 Hz), δ 2.19-2.18 (1H, t, J=2.1 Hz) ppm. ¹³C NMR (100 MHz, CDCl₃) 191.2, 166.4, 135.2, 133.7, 133.6, 133.3, 133.2, 131.8, 131.6, 130.9, 130.7, 128.9, 128.7, 124.4, 123.5, 75.7, 72.3, 33.6 ppm. Anal. Calcd for (C₁₂H₆BrNOS₂): C, 44.45; H, 1.87; N, 4.32. Found: C, 44.37; H, 1.76; N, 4.42.

(Z)-5-(3-bromobenzylidene)-3-(2,2-dimethoxyethyl)-2-thioxothiazolidin-4-one (3d). Yellow solid. Mp 124-126° C. ¹H NMR (400 MHz, CDCl₃) δ 7.53-7.52 (2H, m), δ 7.48-7.46 (1H, d, J=7.8 Hz), δ 7.33-7.31 (1H, d, J=7.8 Hz), δ 7.28-7.24 (1H, t, J=7.8 Hz), δ 4.85-4.82 (1H, t, J=5.6 Hz), 4.19-4.18 (2H, d, J=5.6 Hz), 3.31 (6H, s) ppm. ¹³C NMR (100 MHz, CDCl₃) 193.0, 167.4, 135.3, 133.5, 133.2, 131.2, 130.7, 128.8, 124.5, 123.5, 99.3, 53.9, 45.0 ppm. Anal. Calcd for (C₁₄H₁₄BrNO₃S₂): C, 43.30; H, 3.63; N, 3.61. Found: C, 43.24; H, 3.60; N, 3.71.

(Z)-5-(3-bromobenzylidene)-3-butyl-2-thioxothiazolidin-4-one (3e). Yellow solid. Mp 113-115° C. ¹H NMR (400 MHz, CDCl₃) 7.51-7.45 (3H, m), 7.33-7.24 (2H, m), 4.05-4.02 (2H, t, J=7.5 Hz), 1.61-1-59 (2H, m, J=7.5 Hz), 1.32-1.29 (2H, m, J=7.5 Hz), 0.90-0.87 (3H, t, J=7.2 Hz) ppm. ¹³C NMR (100 MHz, CDCl₃) 192.7, 167.5, 135.4, 133.3, 133.2, 130.7, 128.7, 124.9, 123.4, 44.6, 29.0, 20.1, 13.7 ppm. Anal. Calcd for (C₁₄H₁₄BrNOS₂): C, 47.19; H, 3.96; N, 3.93. Found: C, 47.28; H, 3.88; N, 3.98.

(Z)-5-(3-methoxybenzylidene)-3-phenethyl-2-thioxothiazolidin-4-one (3f). Yellow solid. Mp 122-125. ¹H NMR (400 MHz, CDCl₃) 7.60 (1H, s), 7.34-7.18 (6H, m), 7.03-7.01 (1H, d), 6.92 (2H, s), 4.29-4.25 (2H, t, J=7.9 Hz), 3.79 (3H, s), 2.96-2.92 (2H, t, J=7.9 Hz) ppm. ¹³C NMR (100 MHz, CDCl₃) 193.1, 167.5, 160.1, 137.5, 134.6, 133.1, 130.4, 129.0, 128.9, 128.7, 128.6, 126.8, 123.2, 116.9, 115.2, 55.4, 45.7, 32.9 ppm. Anal. Calcd for (C₁₉H₁₇NO₂S₂): C, 64.20; H, 4.82; N, 3.94. Found: C, 64.31; H, 4.74; N, 3.85.

(Z)-5-((furan-2-yl)methylene)-3-phenethyl-2-thioxothiazolidin-4-one (3g). Orange solid. Mp 150-152° C. ¹H NMR (400 MHz, CDCl₃) 7.63 (1H, s), 7.38 (1H, s), 7.3-7.1 (5H, m), 6.76 (1H, m), 6.52 (1H, m), 4.27-4.23 (2H, t, J=7.9 Hz), 2.95-2.91 (2H, t, J=7.9 Hz) ppm. ¹³C NMR (100 MHz, CDCl₃) 194.3, 167.3, 150.2, 147.0, 137.6, 129.0, 128.6, 126.8, 120.9, 118.6, 118.3, 113.5, 45.6, 33.0 ppm. Anal. Calcd for (C₁₆H₁₃NO₂S₂): C, 60.93; H, 4.15; N, 4.44. Found: C, 60.89; H, 4.24; N, 4.35.

(Z)-3-phenethyl-5-((pyridin-3-yl)methylene)-2-thioxothiazolidin-4-one (3h). Yellow solid. Mp 138-140° C. ¹H NMR (400 MHz, CDCl₃) 8.69 (1H, s), 8-57-8.56 (1H, d, J=4.4 Hz), 7.70-7.68 (1H, d, J=7.9 Hz), 7.59 (1H, s), 7.36-7.33 (1H, dd, J=4.7 Hz, J=7.9 Hz), 7.26-7.16 (5H, m), 4.28-4.24 (2H, t, J=7.9), 2.95-2.91 (2H, t, J=7.9 Hz) ppm. ¹³C NMR (100 MHz, CDCl₃) 192.0, 167.1, 151.9, 150.8, 137.3, 136.3, 129.4, 128.9, 128.8, 128.7, 126.9, 125.5, 124.0, 45.8, 32.9 ppm. Anal. Calcd for (C₁₇H₁₄N₂OS₂): C, 62.55; H, 4.32; N, 8.58. Found: C, 62.58; H, 4.21; N, 8.50.

(Z)-5-((naphthalen-3-yl)methylene)-3-phenethyl-2-thioxothiazolidin-4-one (3i). Yellow solid. Mp 174-176° C. ¹H NMR (400 MHz, CDCl₃) 7.90-7.79 (5H, m), 7.53-7.46 (3H, m), 7.29-7.19 (5H, m), 4.30-4.26 (2H, t, J=7.9 Hz), 2.98-2.94 (2H, t, J=7.9 Hz) ppm. ¹³C NMR (100 MHz, CDCl₃) 193.1, 167.5, 137.6, 133.9, 133.2, 132.1, 130.8, 129.2, 129.0, 128.9, 128.7, 128.3, 127.8, 127.2, 126.8, 126.2, 45.7, 33.0 ppm. Anal. Calcd for (C₂₂H₁₇NOS₂): C, 70.37; H, 4.56; N, 3.73. Found: C, 70.28; H, 4.46; N, 3.70.

N-(2-((Z)-5-(3-Bromobenzylidene)-4-oxo-2-thioxothiazolidin-3-yl)ethyl)-2-hydroxybenzamide (3j). Yield 69%. Yellow solid. ¹H NMR (400 MHz, DMSO-d₆): δ (ppm) 12.32 (s, 1H), 8.86 (t, J=5.2 Hz, 1H), 7.80 (s, 1H), 7.71 (s, 1H), 7.63 (d, J=7.9 Hz, 1H), 7.57-7.54 (m, 2H), 7.44 (t, J=7.9 Hz, 1H), 7.30 (t, J=7.7 Hz, 1H), 6.78 (t, J=8.6 Hz, 2H), 4.20 (t, J=5.2 Hz, 1H), 3.57 (q, J=5.2 Hz, 1H). ¹³C NMR (100 MHz, DMSO-d₆): δ (ppm) 194.2, 170.2, 167.6, 160.7, 135.9, 133.9, 131.1, 129.0, 128.1, 124.8, 123.1, 115.4, 44.4, 36.7. MS (ESI) m/z: 462.9 [M−H]⁻. Anal. Calcd for (C₁₉H₁₅BrN₂O₃S₂): C, 49.25; H, 3.26; N, 6.05. Found: C, 49.19; H, 3.34; N, 6.12.

N-(2-((Z)-5-(3-Fluorobenzylidene)-4-oxo-2-thioxothiazolidin-3-yl)ethyl)-2-hydroxy-benzamide (3k). Yield 62%. Yellow solid. ¹H NMR (400 MHz, DMSO-d₆): δ (ppm) 12.31 (1H, s), 8.85 (t, J=4.6 Hz, 1H), 7.72 (1H, s), 7.58-7.50 (m, 2H), 7.45-7.37 (m, 2H), 7.31-7.29 (m, 2H), 6.80-6.76 (m, 2H), 4.20 (t, J=4.5 Hz, 2H), 3.56 (q, J=4.6 Hz, 2H). ¹³C NMR (100 MHz, DMSO-d₆): δ (ppm) 193.8, 169.8, 167.2, 164.8, 160.2, 135.4, 133.7, 131.7, 131.6, 130.8, 127.6, 126.0, 125.9, 124.3, 118.6, 117.4, 114.9, 43.9, 36.2. MS (ESI) m/z: 401.5 [M−H]⁻. Anal. Calcd for (C₁₉H₁₅FN₂O₃S₂): C, 56.70; H, 3.76; N, 6.96. Found: C, 56.59; H, 3.83; N, 6.84.

### Example 2

### Chemistry

### Synthesis of pyrrolidine-2,5-dione derivatives

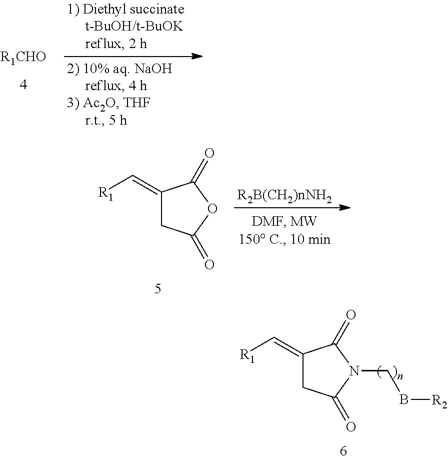

General Procedure for the synthesis of compounds 5: To a solution of potassium tert-butoxide, from 1.13 g (29 mmol, 1.2 eq) of potassium in 15 mL of tert-butyl alcohol at reflux was added 4.86 mL (29 mmol, 1.2 eq) of diethyl succinate in 2.8 mL of tert-butyl alcohol. A solution of the opportune benzaldehyde 4 (1 eq) in 2.6 mL of tert-butyl alcohol was added dropwise and the resulting heterogeneous mixture was refluxed for 2 h and then at r.t. overnight. After cooling, 30 mL of water was added and tert-butyl alcohol was removed by distillation. To this mixture a solution of 3.12 g (55.6 mmol, 2.3 eq) of KOH in 10 mL of water was added and mixture was heated at reflux for 4 h. The mixture was washed with Et₂O. The acqueous layer was diluited with water and conc. HCl was added up to pH 1. Then it was stirred 5 min. and it was extracted with Et₂O, dried over Na₂SO₄ and then solvent was removed. The crude product was recrystallized from cyclo hexane/hexane.

The latter intermediate (1.31 g, 5.87 mmol, 1 eq) was dissolved in THF, acetic anhydride (1.22 mL, 2.2 eq) was added and the resulting mixture stirred at room temperature for 5 h. Then THF was removed and tert-butyl methylether (MTB-ether) was added to induce a more complete crystallization. The crystals are filtered, washed with MTB-ether and dried in vacuo.

General Procedure for the synthesis of compounds 6: Compounds with general structure 5 and the opportune amine (1 eq.) were suspended in a DMF (3 mL) and the mixture was heated with MW at 150° C. for 10 min. Then water was added and the solution was extracted with CH₂Cl₂. The organic layers were collected, washed with brine and dried over Na₂SO₄. The solvent was removed and residue was crystallized in EtOH to obtain the final product.

(E)-2-(3-fluorobenzylidene)succinic acid: ¹H NMR (400 MHz, DMSO-d₆) δ 3.39 (s, 2H), δ 7.08-7.2 (m, 3H), δ 7.37-4.43 (m, 1H), δ 7.63 (m, 1H), δ 12.48 (br s, 2H) ppm. MS (ESI) m/z: 223 [M−H]⁻. Anal. Calcd for (C₁₁H₉FO₄): C, 58.93; H, 4.05. Found: C, 58.90; H, 3.98.

(E)-2-(3-bromobenzylidene)succinic acid: ¹H NMR (400 MHz, DMSO-d₆) δ 3.26 (s, 2H), δ 7.32-7.33 (m, 2H), δ 7.48-7.53 (m, 2H), δ 7.62 (s, 1H), δ 12.55 (br s, 2H) ppm. MS (ESI) m/z: 284 [M−H]⁻. Anal. Calcd for (C₁₁H₉BrO₄): C, 46.34; H, 3.18. Found: C, 46.38; H, 3.28.

(E)-3-(3-fluorobenzylidene)-dihydrofuran-2,5-dione (5a): ¹H NMR (400 MHz, DMSO-d₆) δ 3.93-3.94 (d, 2H), δ 7.23-7.29 (m, 1H), δ 7.42-7.48 (m, 3H), 7.57 (s, 1H) ppm. MS (ESI) m/z: 205 [M−H]⁻. Anal. Calcd for (C₁₁H₇FO₃): C, 64.08; H, 3.42. Found: C, 64.12; H, 3.38.

(E)-3-(3-bromobenzylidene)-dihydrofuran-2,5-dione (5b): ¹H NMR (400 MHz, DMSO-d₆) δ 3.94-3.95 (d, 2H), δ 7.53-7.62 (m, 3H), δ 7.80 (s, 1H) ppm. MS (ESI) m/z: 266 [M−H]⁻. Anal. Calcd for (C₁₁H₇BrO₃): C, 49.47; H, 2.64. Found: C, 49.56; H, 2.66.

3-((E)-3-(3-fluorobenzylidene)-2,5-dioxopyrrolidin-1-yl)-N-(2-hydroxyphenyl)propanamide (6a): ¹H NMR (400 MHz, DMSO-d₆) δ 2.59-2.65 (t, 2H, J=6.3 Hz), δ 3.66 (s, 2H), δ 3.68-3.65 (t, 2H, J=6.3 Hz), δ 6.63-6.70 (t, 1H, J=7.2 Hz), δ 6.74-6.78 (d, 1H), δ 6.82-6.88 (t, 1H, J=7.2 Hz), 7.15-7.23 (d, 1H), 7.38-7.46 (m, 4H), 7.53-7.58 (d, 1H), 9.29 (s, 1H), 9.55 (s, 1H) ppm. ¹³C NMR (100 MHz, DMSO-d₆) 34.1, 34.3, 35.4, 116.2, 116.8, 117.1, 119.3, 123.5, 125.3, 126.4, 126.8, 127.3, 131.1, 131.3, 136.9, 148.7, 161.5, 169.5, 170.7, 174.4 ppm. MS (ESI) m/z: 367 [M−H]⁻. Anal. Calcd for (C₂₀H₁₇FN₂O₄): C, 65.21; H, 4.65; N, 7.60. Found: C, 65.14; H, 4.72; N, 7.66.

3-((E)-3-(3-bromobenzylidene)-2,5-dioxopyrrolidin-1-yl)-N-(2-hydroxyphenyl)propanamide (6b): ¹H NMR (400 MHz, DMSO-d₆) δ 2.61 (t, 2H, J=7.1Hz), δ 3.66 (s, 2H), δ 3.71 (t, 2H, J=7.1 Hz), δ 6.67 (t, 1H, J=7.4 Hz), δ 6.76 (d, 1H, J=7.6 Hz), 6.86 (t, 1H, J=7.4 Hz), 7.31-7.41 (m, 2H), 7.53-7.60 (m, 3H), 7.77 (s, 1H), 9.28 (s, 1H), 9.54 (s, 1H) ppm. ¹³C NMR (100 MHz, DMSO-d₆) δ3.5, 33.6, 34.8, 115.7, 118.8, 122.2, 123.9, 124.6, 125.3, 126.9, 128.7, 130.3, 130.9, 132.3, 132.5, 136.4, 148.2, 168.2, 170.1, 173.8 ppm. MS (ESI) m/z: 428.3 [M−H]⁻. Anal. Calcd for (C₂₀H₁₇BrN₂O₄): C, 55.96; H, 3.99; N, 6.53. Found: C, 55.88; H, 4.06; N, 6.46.

### Example 3

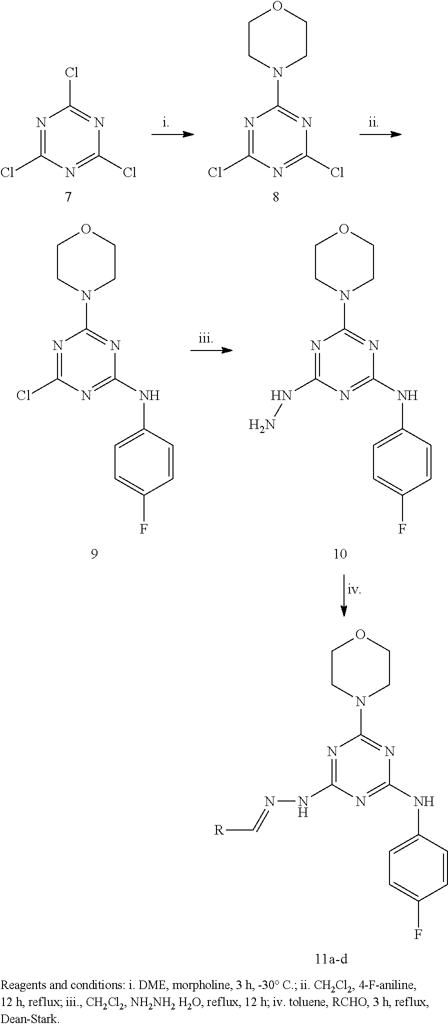

4-(4,6-dichloro-1,3,5-triazin-2-yl)morpholine (8): To a stirred solution of cyanuric chloride 7 (1 g; 5.42 mmol) in dimethoxy ethane (50 mL) at 30° C., morpholine (470.1 μL; 5.42 mmol) was added dropwise. The reaction mixture was vigorously stirred for 3 h at −30° C. then warmed up to room temperature and washed with HCl 3N, water, brine and finally dried over anhydrous Na₂SO₄. The product is obtained as a white solid used in the next step without further purification.

Yield=79%; R_{f} (Et₂O/Hex: 2:1)=0.62; Mp 156.7-156.9° C.; ¹H-NMR (400 MHz, CD₃OD): δ (ppm) 3.80 (t, J=4.59 Hz, 4H); 3.66 (t, J=4.59 Hz, 4H); ¹³C-NMR (100 MHz, CDCl₃): δ (ppm) 170.44, 164.11, 66.37, 44.47; MS (ESI): m/z 235.1 [M+H]⁺, 257.2 [M+Na]⁺; Anal. Calcd for (C₇H₈Cl₂N₄O): C, 35.77; H, 3.43; N, 23.83. Found: C, 35.74; H, 3.38; N, 23.92.

4-chloro-N-(4-fluorophenyl)-6-morpholino-1,3,5-triazin-2-amine (9): To a stirred solution of compound 8 (650 mg, 2.76 mmol) in dichloromethane, 4-fluoro aniline (310.81 μL, 2.71 mmol) was added. The reaction mixture was refluxed for 12 h and then washed with HCl 3N, water, brine and finally dried over anhydrous Na₂SO₄. The organic phase was evaporated to dryness and the white residue was dissolved with the minimum amount of dichloromethane; by addition of petroleum ether the final compound precipitated as a white solid and was collected by filtration.

Yield=90%; R_{f} (Et₂O:Hex 2:1)=0.51; Mp 170-172° C.; ¹H-NMR (400 MHz, CDCl₃): δ (ppm) 7.38 (q, J=8.60, 2H); 6.97 (t, J=8.63 Hz, 2H); 3.78 (brs, 2H); 3.72 (brs, 2H); 3.67-3.66 (m, 4H); MS (ESI): m/z 310.7 [M+H]⁺; 332.6 [M+Na]⁺; Anal. Calcd for (C₁₃H₁₃ClFN₅O): C, 50.41; H, 4.23; N, 22.61. Found: C, 50.35; H, 4.19; N, 22.69.

N-(4-fluorophenyl)-4-hydrazinyl-6-morpholino-1,3,5-triazin-2-amine (10): To a solution of 9 (900 mg; 2.69 mmol) in dichloromethane, hydrazine (544.2 μL, 10.76 mmol) was added and the resulting mixture was refluxed for 12 h. After cooling down to room temperature, the mixture was washed with water, brine and finally dried over anhydrous Na₂SO₄. The organic phase was evaporated to dryness and the residue was dissolved with the minimum amount of dichloromethane; by addition of petroleum ether the final compound precipitated as a white solid and was collected by filtration.

Yield=70%; R_{f} (CH₂Cl₂:CH₃OH 95:5)=0.54; MS (ESI): m/z 306 [M+H]⁺; Anal. Calcd for (C₁₃H₁₆FN₇O): C, 51.14; H, 5.28; N, 32.11. Found: C, 51.07; H, 5.33; N, 32.02.

### Synthesis of the Final Compounds 11a-c.

General procedure: To a stirred solution of 10 in toluene, the opportune aldehyde (2 eq) was added and the resuting mixture was refluxed for 3 h using a Dean-Stark apparatus for azeotropical removal of water. The reaction mixture was evaporated to dryness and the resulting residue was dissolved with the minimum amount of dichloromethane; by addition of petroleum ether the final compound precipitated and collected by filtration.

(E)-N-(4-fluorophenyl)-4-morpholino-6-(2-(naphthalen-1-ylmethylene)hydrazinyl)-1,3,5-triazin-2-amine (11a): Yield=80%; R_{f} (CH₂Cl₂:MeOH 95:5)=0.53; Mp 220.0-220.4° C.; ¹H-NMR (400 MHz, CDCl₃): δ (ppm) 8.62 (1H); 8.47 (1H); 7.91-7.81 (m, 3H); 7.53-7.49 (m, 5H); 6.99 (t, J=8.4; 3H); 3.82 (s, 4H); 3.76 (s, 4H); ¹³C-NMR (100 MHz, CDCl₃): δ (ppm) 165.85, 161.14, 156.33, 142.59, 133.83, 128.77, 127.56, 127.03, 126.82, 126.26, 121.81, 115.14, 77.62, 76.99, 76.35, 66.75, 43.80; MS (ESI): m/z 444.2 [M+H]⁺; Anal. Calcd for (C₂₄H₂₂FN₇O): C, 65.00; H, 5.00; N, 22.11. Found: C, 65.06; H, 5.10; N, 22.02.

(E)-N-(4-fluorophenyl)-4-morpholino-6-(2-(naphthalen-2-ylmethylene)hydrazinyl)-1,3,5-triazin-2-amine (11b): Yield=83%; R_{f} (CH₂Cl₂:MeOH 95:5)=0.62; Mp 225-226° C.; ¹H-NMR (400 MHz, CDCl₃): δ (ppm) 10.95 (brs, 1H); 8.22 (s, 1H); 7.93-7.83 (m, 6H); 7.46-7.44 (m, 2H); 7.05 (t, J=8.5; 2H); 3.68 (s, 4H); 3.58 (s, 4H); ¹³C-NMR (100, MHz, CDCl₃): δ (ppm) 165.26, 164.62, 158.94, 156.57, 142.89, 137.18, 133.80, 133.48, 133.44, 133.28, 128.83, 128.59, 128.21, 128.08, 127.14, 122.92, 121.76, 115.41, 115.19, 66.49, 43.90; MS (ESI): m/z 444.5 [M+H]⁺; Anal. Calcd for (C₂₄H₂₂FN₇O): C, 65.00; H, 5.00; N, 22.11. Found: C, 65.05; H, 5.09; N, 22.14.

(E)-4-(2-(2,6-difluorobenzylidene)hydrazinyl)-N-(4-fluorophenyl)-6-morpholino-1,3,5-triazin-2-amine (11c): Yield=85%; R_{f} (CH₂Cl₂:MeOH: 95:5)=0.65; Mp 155.5-156° C.; ¹H-NMR (CDCl₃): δ (ppm) 8.61 (s, 1H); 7.95 (s, 1H); 7.45 (brs, 3H); 7.23-7.10 (m, 1H); 6.96-6.91 (t, J=8.5 Hz, 1H); 6.88-6.83 (t, J=8.5 Hz, 2H); 3.75 (s, 4H); 3.68 (s, 4H); ¹³C-NMR (CDCl₃): δ (ppm) 163.60, 163.47, 161.16, 158.60, 156.35, 133.33, 130.48, 121.75. 115.59, 115.15, 112.07, 111.68, 77.63, 77.20, 76.36, 66.70, 43.83; MS (ESI): m/z 430 [M+H]⁺; Anal. Calcd for (C₂₀H₁₈F₃N₇O): C, 55.94; H, 4.23; N, 22.83. Found: C, 55.96; H, 4.29; N, 22.88

(E)-2-((2-(4-(4-Fluorophenylamino)-6-morpholino-1,3,5-triazin-2-yl)hydrazono)methyl)phenol (11d). Mp 234° C. ¹H NMR (400 MHz, CDCl₃-d): δ (ppm) 11.37 (brs, 1H), 7.84 (s, 1H), 7.42 (brs, 1H), 7.22-7.19 (m, 1H), 7.09-7.07 (m, 1H), 6.98-6.93 (m, 3H), 6.84-6.80 (m, 1H), 3.77-3.68 (m, 8H). MS (ESI) m/z: 410.4 [M+H]⁺; 432 [M+Na]⁺. Anal. Calcd for (C₂₀H₂₀FN₇O₂): C, 58.67; H, 4.92; N, 23.95. Found: C, 58.71; H, 4.86; N, 23.99.

### Example 4

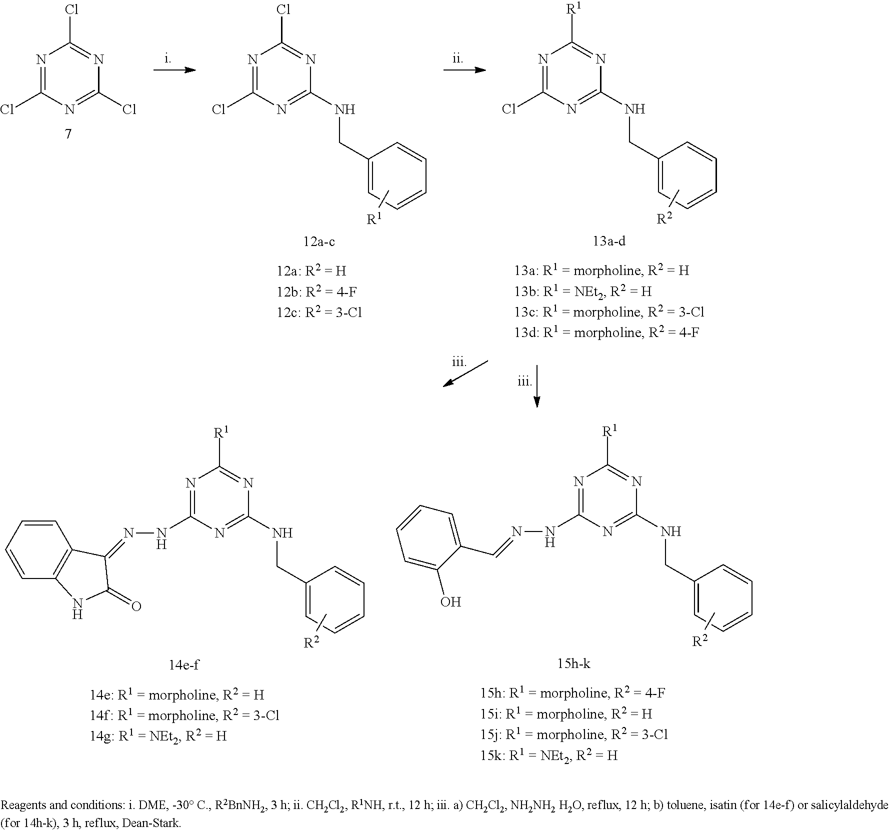

General procedure for the synthesis of compound 12a-c. To a stirred solution of cyanuric chloride 7 (1 g, 5.42 mmol) in dimethoxyethane (35 mL) at −30° C., the opportune benzylamine (1 eq) was added dropwise. The reaction mixture was vigorously stirred for 3 h at −30° C. then, warmed up to room temperature and washed with 3N HCl, water, brine and finally dried over anhydrous Na₂SO₄. The organic phase was evaporated to dryness and the white residue was dissolved with the minimum amount of dichloromethane; by addition of petroleum ether, the final compounds precipitated and were collected by filtration.

N-Benzyl-4,6-dichloro-1,3,5-triazin-2-amine (12a). Yield 85%. ¹H NMR (200 MHz, CDCl₃-d): δ (ppm) 7.30-7.26 (m, 5H), 4.48 (m, 2H), MS (ESI): m/z: 256.1 [M+H]⁺. Anal. Calcd for (C₁₀H₉Cl₂N₄): C, 47.08; H, 3.16; N, 21.96. Found: C, 47.02; H, 3.21; N, 22.04.

4,6-Dichloro-N-(4-fluorobenzyl)-1,3,5-triazin-2-amine (12b): Yield 75%. Mp 151.0-151.5° C. ¹H NMR (400 MHz, CD₃OD-d₄): δ (ppm) 7.23-7.19 (m, 2H), 6.98 (t, J=8.48, 2H), 6.19 (brs, 1H). MS (ESI) m/z: 274.1 [M+H]⁺. Anal. Calcd for (C₁₀H₇Cl₂FN₄): C, 43.98; H, 2.58; N, 20.52. Found: C, 43.88; H, 2.62; N, 20.59.

4,6-Dichloro-N-(3-chlorobenzyl)-1,3,5-triazin-2-amine (12c): Yield 78%. Mp 149° C. ¹H NMR (400 MHz, CD₃OD-d₄): δ (ppm) 7.23-7.19 (m, 3H), 7.13-7.10 (m, 1H), 6.58 (brs, 1H), 4.60 (d, J=6.29, 2H). ¹³C NMR (100 MHz, CDCl₃-d): δ (ppm) 169.95, 165.98, 138.48, 134.76, 130.18, 128.24, 127.70, 125.73, 44.68. MS (ESI) m/z: 289.5 [M+H]⁺; 311.5 [M+Na]⁺. Anal. Calcd for (C₁₀H₇Cl₃N₄): C, 41.48; H, 2.44; N, 19.35. Found: C, 41.45; H, 2.38; N, 19.36.

General procedure for the synthesis of compounds 13a-d. To a suspension of the opportune intermediate 12a-c (1 eq) in dichloromethane, the opportune amine (2 eq) was added dropwise. The reaction mixture was stirred at room temperature for 12 h and then washed with 3N HCl, water, brine and finally dried over anhydrous Na₂SO₄. The organic phase was evaporated to dryness and the white residue was dissolved with the minimum amount of dichloromethane; by addition of petroleum ether the final compounds precipitated and were collected by filtration.

N-Benzyl-4-chloro-6-morpholino-1,3,5-triazin-2-amine (13a). Yield 67%. ¹H NMR (200 MHz, CDCl₃-d): δ (ppm) 7.28-7.25 (m, 5H), 7.07 (brs, 1H), 4.58 (d, J=6 Hz, 2H), 3.74-3.65 (m, 8H). MS (ESI): m/z 306.8 [M+H]⁺. Anal. Calcd for (C₁₄H₁₆ClN₅O): C, 54.99; H, 5.27; N, 22.90. Found: C, 55.05; H, 5.25; N, 22.87.

N2-Benzyl-6-chloro-N4,N4-diethyl-1,3,5-triazine-2,4-diamine (13b): Yield 75%. ¹H NMR (200 MHz, CDCl₃-d): δ (ppm) 7.29-7.24 (m, 5H), 6.06 (brs, 1H), 4.57 (s, 2H), 3.55-3.48 (m, 4H), 1.16-1.07 (m, 6H). MS (ESI) m/z: 292.8 [M+H]⁺. Anal. Calcd for (C₁₄H₁₅Cl₂N₅): C, 57.63; H, 6.22; N, 24.00. Found: C, 57.60; H, 6.16; N, 24.08.

4-Chloro-N-(3-chlorobenzyl)-6-morpholino-1,3,5-triazin-2-amine (13c): Yield 85%. Mp 168-168.5° C. ¹H NMR (400 MHz, CD₃OD-d₄): δ (ppm) 7.72 (s, 1H), 7.19-7.16 (m, 2H), 6.67 (brs, 1H), 4.54-4.49 (m, 2H), 3.72-3.61 (m, 8H). ¹³C NMR (100 MHz, CDCl₃-d): δ (ppm) 165.46, 164.34, 163.10, 144.97, 134.07, 129.34, 128.90, 128.82, 115.42, 115.21, 66.54, 66.37, 44.03, 43.79. MS (ESI) m/z: 340.1 [M+H]⁺. Anal. Calcd for (C₁₄H₁₅Cl₂N₅): C, 49.43; H, 4.44; N, 20.59. Found: C, 49.40; H, 4.46; N, 20.51.

4-Chloro-N-(4-fluorobenzyl)-6-morpholino-1,3,5-triazin-2-amine (13d): Yield 95%. Mp 192° C. ¹H NMR (400 MHz, CD₃OD-d₄): δ (ppm) 7.23-7.17 (m, 2H), 6.95 (t, J=8.43, 2H), 6.00 (brs, 1H), 4.52-4.47 (m, 2H), 3.72 (s, 4H), 3.62 (s, 4H). MS (ESI) m/z: 324.7 [M+H]⁺. Anal. Calcd for (C₁₄H₁₅ClFN₅O): C, 51.94; H, 4.67; N, 21.63. Found: C, 51.88; H, 4.74; N, 21.60.

General procedure for the synthesis of compounds 14e-g and 15h-k. To a solution of the opportune intermediate 13a-d (1 eq) in dichloromethane, hydrazine (4 eq) was added and the resulting mixture was refluxed for 12 h. After cooling down to room temperature, the mixture was washed with water, brine and finally dried over anhydrous Na₂SO₄. The organic phase was evaporated to dryness, the residue was dissolved in toluene and reacted with the opportune aldehyde (2 eq). The reaction mixture was refluxed for 3 h using a Dean-Stark apparatus for azeotropical removal of water. The reaction mixture was evaporated to dryness and the resulting residue was dissolved with the minimum amount of dichloromethane; by addition of petroleum ether the desired compounds 14e-g and 15h-k precipitated and were collected by filtration.

(E)-3-((2-(4-(Benzylamino)-6-morpholino)-1,3,5-triazin-2-yl)hydrazono)indolin-2-one (14e). Yield 80%. ¹H NMR (400 MHz, CDCl₃-d): δ (ppm), 12.39 (s, 1H), 8.87 (s, 1H), 7.60-7.58 (m, 1H), 7.34-7-30 (m, 6H), 6.96-6.95 (m, 1H), 6.83-6.81 (m, 1H), 6.15 (brs, 1H), 4.64 (s, 2H), 3.60-3.57 (m, 4H), 1.26-1.12 (m, 6H). ¹³C NMR (CDCl₃-d): δ (ppm) 164.42, 163.26, 141.17, 139.76, 138.15, 137.82, 129.86, 127.88, 126.80, 126.38, 122.17, 120.82, 120.17, 110.26, 43, 99, 40.93, 12.03. MS (ESI) m/z: 431.5 [M+H]⁺. Anal. Calcd for (C₂₂H₂₂N₈O₂): C, 61.38; H, 5.15; N, 26.03. Found: C, 61.34; H, 5.21; N, 26.09.

(Z)-3-(2-(4-(3-Chlorobenzylamino)-6-morpholino-1,3,5-triazin-2-yl)hydrazono)indolin-2-one (14f). Yield 71%. Mp 144-144.5° C. ¹H NMR (400 MHz, CD₃OD-d₄): δ (ppm) 12.39 (s, 1H), 8.15 (s, 1H), 7.62 (d, J=6.80, 1H), 7.26 (s, 1H), 7.16 (m, 4H), 7.00-6.90 (m, 1H), 6.77 (d, J=7.82), 4.53 (s, 2H), 3.75 (s, 4H), 3.65 (s, 4H). ¹³C NMR (100 MHz, CDCl₃-d): δ (ppm) 165.08, 164.26, 163.10, 141.13, 139.84, 134.33, 132.42, 130.15, 129.75, 127.68, 127.32, 125.50, 122.93, 121.33, 120.88, 110.47, 66.76, 44.40, 43.77. MS (ESI) m/z: 465.9 [M+H]⁺. Anal. Calcd for (C₂₂H₂₁ClN₈O₂): C, 56.84; H, 4.55; N, 24.10. Found: C, 56.88; H, 4.47; N, 24.11.

(E)-3-(2-(4-(benzylamino)-6-(diethylamino)-1,3,5-triazin-2-yl)hydrazono)indolin-2-one (14g) Yield 78%. ¹H NMR (400 MHz, CDCl₃-d): δ (ppm) 11.52 (s, 1H), 7.92 (s, 2H), 7.33 (s, 3H), 7.18-7.17 (m, 1H), 7.01-6.99 (m, 1H), 6.91-6.87 (m, 1H), 4.63 (s, 2H), 3.82-3.73 (m, 8H). MS (ESI) m/z: 417.5 [M+H]⁺. Anal. Calcd for (C₂₂H₂₄N₈O): C, 63.45; H, 5.81; N, 26.90. Found: C, 63.53; H, 5.88; N, 26.81.

(E)-2-((2-(4-(4-fluorobenzylamino)-6-morpholino-1,3,5-triazin-2-yl)hydrazono)methyl)phenol (15h). Yield 87%. Mp 114-114.5° C. ¹H NMR (400 MHz, CD₃OD-d₄): δ (ppm) 11.48 (s, 1H), 7.77 (s, 1H), 7.19 (s, 3H), 7.02 (s, 1H), 6.91 (s, 3H), 6.80 (t, J=6.63 Hz, 1H), 4.47 (s, 2H), 3.74 (s, 4H), 3.65 (s, 4H). ¹³C NMR (100 MHz, CDCl₃-d): δ (ppm) 165.07, 163.85, 163.31, 160.82, 158.07, 143.85, 134.75, 130.81, 129.78, 129.19, 119.18, 118.15, 117.02, 115.51, 115.30, 66.81, 44.13, 43.72. MS (ESI) m/z: 424.4 [M+H]⁺; 446.4 [M+Na]⁺. Anal. Calcd for (C₂₁H₂₂FN₇O₂): C, 59.57; H, 5.24; N, 23.15. Found: C, 59.56; H, 5.06; N, 23.14.

(E)-2-((2-(4-(Benzylamino)-6-morpholino-1,3,5-triazin-2-yl)hydrazono)methyl)phenol (15i). Yield 74%. ¹H NMR (400 MHz, CDCl₃-d): δ (ppm) 12.47 (s, 1H), 9.27 (s, 1H), 7.51-7.49 (m, 1H), 7.32-7.23 (m, 5H), 7.15-7.13 (m, 1H), 7.05-7.01 (m, 1H), 6.90-6.79 (m, 1H), 6.48 (brs, 1H), 4.64 (s, 2H), 3.84-3.72 (m, 8H). ¹³C NMR (CDCl₃-d): δ (ppm) 166.07, 165.05, 164.13, 163.32, 139.96, 138.83, 132.51, 129.99, 128.40, 127.41, 127.10, 122.74, 121.16, 120.72, 110.48, 66.70, 44.88, 43.68. MS (ESI) m/z: 406.5 [M+H]⁺. Anal. Calcd for (C₂₁H₂₃N₇O₂): C, 62.21; H, 5.72; N, 24.18. Found: C, 62.26; H, 5.64; N, 24.15.

(E)-2-((2-(4-(3-chlorobenzylamino)-6-morpholino-1,3,5-triazin-2-yl)hydrazono)methyl)phenol (15j). Yield 82%. Mp 163.5-164° C. ¹H NMR (400 MHz, CD₃OD-d₄): δ (ppm) 11.51 (s, 1H), 7.83 (s, 1H), 7.29-7.07 (m, 3H), 6.96 (d, J=8.2, 1H), 6.84 (t, J=7.4, 3H), 4.54 (d, J=4.9 Hz, 2H), 3.77 (s, 4H), 3.71 (s, 4H). ¹³C NMR (100 Hz, CDCl₃-d): δ (ppm) 164.84, 158.00, 143.93, 141.09, 134.32, 130.75, 129.76, 127.57, 127.34, 125.48, 119.10, 118.04, 116.93, 66.68, 44.16, 43.66. MS (ESI) m/z: 440.9 [M+H]⁺; 462.9 [M+Na]⁺. Anal. Calcd for (C₂₁H₂₂ClN₇O₂): C, 57.34; H, 5.04; N, 22.29. Found: C, 57.37; H, 5.12; N, 22.21.

(E)-2-((2-(4-(benzylamino)-6-(diethylamino)-1,3,5-triazin-2-yl)hydrazono)methyl)phenol (15k). Yield 85%. ¹H NMR (400 MHz, CDCl₃-d): δ (ppm) 11.54 (brs, 1H), 7.86 (s, 1H), 7.31-7.19 (m, 6H), 7.10-7.08 (m, 1H), 6.97-6.95 (m, 1H), 6.85-6.82 (m, 1H), 5.24 (brs, 1H), 4.59-4.57 (m, 2H), 3.56 (m, 4H), 1.18-1.11 (m, 6H). MS (ESI) m/z: 392.5 [M+H]⁺. Anal. Calcd for (C₂₁H₂₅N₇O): C, 64.43; H, 6.44; N, 25.05. Found: C, 64.38; H, 6.48; N, 25.00.

### Example 5

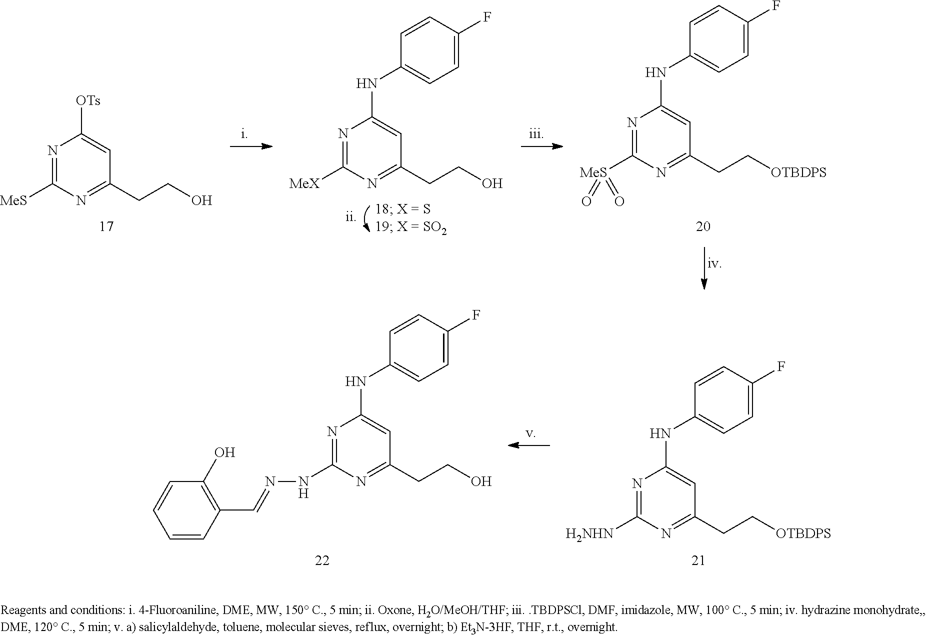

2-(6-(4-Fluorophenylamino)-2-(methylthio)pyrimidin-4-yl)ethanol (18). A mixture of 17 (0.28 mmol) and 4-fluoroaniline (1.12 mmol) in dimethoxyethane was irradiated under microwave at 150° C. for 5 min. The reaction mixture was then concentrated under reduced pressure, diluted with EtOAc and washed with water, dried and purified by flash chromatography (DCM/EtOAc:1/1) to get the desired product 18.

Yield 80%. ¹H NMR (400 MHz, CDCl₃-d): δ (ppm) 7.3 (m, 2H), 7.1 (t, 2H), 6.62 (s, 1H), 6.1 (s, 1H), 3.95 (t, J=8 Hz, 2H), 2.75 (t, J=8 Hz, 2H), 2.5 (s, 3H). ¹³C NMR (50 MHz, CD3OD-d₄: δ (ppm) 172.75, 167.07, 162.66, 162.06, 157.88, 136.88, 123.84, 123.69, 116.42, 115.97, 101.83, 61.56, 40.98, 14.16. MS (ESI) m/z: 280.3 [M+H]⁺. Anal. Calcd for (C₁₃H₁₄FN₃OS): C, 55.90; H, 5.05; N, 15.04. Found: C, 55.95; H, 5.12; N, 15.08.

2-(6-(4-Fluorophenylamino)-2-(methylsulfonyl)pyrimidin-4-yl)ethanol (19). Compound 18 (0.448 mmol) was dissolved in a 1:1 mixture of MeOH and H₂O (6 mL); Oxone (0.896 mmol) in 3 mL of water was added portionwise and the mixture was then stirred overnight at r.t. The crude mixture was concentrated under reduced pressure, neutralized with NaHCO₃ solution and washed with EtOAc. The organic extracts were collected, dried over Na₂SO₄, and then evaporated under reduced pressure. The crude residue was purified by flash chromatography (CH₂Cl₂:AcOEt 1:1) to give the desired product 19.

Yield=71%. ¹H NMR (200 MHz, CDCl₃-d): δ (ppm) 7.55 (br, 1H), 7.35 (m, 2H), 7.1 (m, 2H), 6.55 (s, 1H), 3.95 (t, J=8 Hz, 2H), 3.3 (s, 3H), 2.85 (t, J=8 Hz, 3H). ¹³C NMR (50 MHz, CD₃OD-d₄: δ (ppm) 168.72, 166.44, 163.15, 162.83, 158.83, 136.07, 124.13, 116.78, 116.33, 108.58, 61.11, 41.14, 39.31. MS (ESI) m/z: 312.3 [M+H]⁺. Anal. Calcd for (C₁₃H₁₄FN₃O₃S): C, 50.15; H, 4.53; N, 13.50. Found: C, 50.18; H, 4.47; N, 13.59.

6-(2-(tert-Butyldiphenylsilyloxy)ethyl)-N-(4-fluorophenyl)-2(methylsulfonyl)pyrimidin-4-amine (20). Compound 19 (0.16 mmol) and tert-butyldiphenylsilylchloride (0.16 mmol) were dissolved in dry DMF and the mixture was irradiated under microwave at 100° C. for 5 min. The reaction mixture was diluted with EtOAc, washed several times with water, brine, dried over Na₂SO₄. The crude material was purified by flash chromatography (DCM/EtOAc:9/1) to give the desired product 20.

Yield=80%. ¹H NMR (200 MHz, CDCl₃-d): δ (ppm) 7.55 (m, 4H), 7.40 (m, 6H), 7.25 (m, 2H), 7.05 (m, 2H), 6.60 (s, 1H), 4.00 (t, J=8 Hz, 2H), 3.20 (s, 3H), 2.75 (t, J=8 Hz, 2H), 0.95 (s, 9H). ¹³C NMR (50 MHz, CDCl₃-d): δ (ppm) 168.95, 165.20, 163.10, 162.16, 158.10, 135.35, 133.18, 129.74, 127.68, 125.61, 125.46, 116.76, 116.30, 105.22, 61.98, 40.65, 38.79, 29.63, 26.67. MS (ESI) m/z: 550.7 [M+H]⁺. Anal. Calcd for (C₂₉H₃₂FN₃O₃SSi): C, 63.36; H, 5.87; N, 7.64. Found: C, 63.43; H, 5.80; N, 7.60.

6-(2-(tert-Butyldiphenylsilyloxy)ethyl)-N-(4-fluorophenyl)-2-hydrazinylpyrimidin-4-amine (21). Compound 20 (0.2 mmol) was dissolved in dimethoxyethane (2 mL) and the resulting mixture was irradiated under microwave at 120° C. for 5 min. The solvent was evaporated under reduced pressure and the remaining crude material was purified by flash chromatography (DCM/MeOH: 9.5/0.5) to give the desired product 21.

Yield=90%. ¹H NMR (400 MHz, CDCl₃-d): δ (ppm) 7.50 (d, J=4 Hz, 4H), 7.27 (m, 6H), 7.2 (m, 2H), 6.95 (t, J=8 Hz, 2H), 5.87 (s, 1H); 3.89 (t, J=8 Hz, 2H), 2.61 (t, J=8 Hz, 2H), 0.91 (s, 9H). ¹³C NMR (100 MHz, CDCl₃-d): δ (ppm) 161.98, 161.02, 158.58, 135.43, 134.82, 134.50, 133.65, 129.62, 127.75, 127.60, 127.53, 124.81, 124.73, 116.12, 115.89, 95.26, 62.54, 40.77, 29.70, 26.81. MS (ESI) m/z: 502.7 [M+H]⁺. Anal. Calcd for (C₂₈H₃₂FN₅OSi): C, 67.04; H, 6.43; N, 13.96. Found: C, 67.12; H, 6.40; N, 13.94.

2-β2-(4-(2-(tert-Butyldiphenylsilyloxy)ethyl)-6-(4-fluorophenylamino)pyrimidin-2-yl) hydrazono)methyl)phenol. Compound 21 (0.046 mmol) and salicylaldehyde (0.046 mmol) were dissolved in dry toluene containing molecular sieves and the mixture was refluxed for 2 h, then it was cooled to r.t., concentrated under reduced pressure and purified by flash chromatography (DCM/EtOAc:9/1) to give the desired product.

Yield=54%. ¹H NMR (200 MHz, CDCl₃-d): δ (ppm) 8.05 (s, 1H), 7.55 (d, J=4 Hz, 4H), 6.7-7.4 (m, 14H), 5.9 (s, 1H), 3.9 (t, J=8 Hz, 2H), 2.7 (t, J=8 Hz, 2H), 0.95 (s, 9H). ¹³C NMR (50 MHz, CDCl₃-d): δ (ppm) 166.78, 162.35, 162.22, 158.34, 157.97, 143.825, 135.50, 133.94, 133.88, 133.47, 130.50, 129.78, 129.61, 127.63, 124.58, 124.52, 119.13, 118.39, 116.92, 116.42, 115.97, 95.55, 62.24, 39.90, 29.58, 26.77. MS (ESI) m/z: 606.8 [M+H]⁺. Anal. Calcd for (C₃₅H₃₆FN₅O₂Si): C, 69.39; H, 5.99; N, 11.56. Found: C, 69.38; H, 6.05; N, 11.50.

2-((2-(4-(4-Fluorophenylamino)-6-(2-hydroxyethyl)pyrimidin-2-yl)hydrazono)methyl)phenol (22). 2-(2-(4-(2-(tert-Butyldiphenylsilyloxy)ethyl)-6-(4-fluorophenylamino)pyrimidin-2-yl) hydrazono)methyl)phenol (0.025 mmol) was dissolved in dry THF (2 mL), triethylamine trihydrofluoride (24 μL, 0.150 mmol) was added and the resulting mixture was stirred overnight at r.t. NaHCO₃ solution was added to the reaction mixture, then it was extracted with EtOAc, dried over Na₂SO₄ and evaporated. The crude material was dissolved in CH₂Cl₂ and precipitated by petroleum ether to give the pure product 22 without further purification. Yield=73%. ¹H NMR (400 MHz, CD3OD-d₄): δ (ppm) 8.27 (s, 1H), 7.73 (m, 2H), 7.43 (m, 1H), 7.26 (t, J=8 Hz, 1H), 7.1 (t, J=8 Hz, 2H), 6.92 (q, J=8 Hz, 2H), 6.17 (s, 1H), 3.94 (t, J=6 Hz, 2H), 2.8 (t, J=6 Hz, 2H). ¹³C NMR (50 MHz, CDCl₃-d): δ (ppm) 163.22, 158.79, 145.46, 131.71, 130.35, 123.54, 123.49, 120.43, 117.32, 116.55, 116.10, 99.32, 61.44, 39.53, 30.77. MS (ESI) m/z: 368.4 [M+H]⁺. Anal. Calcd for (C₁₉H₁₈FN₅O₂): C, 62.12; H, 4.94; N, 19.06. Found: C, 62.10; H, 4.98; N, 19.00.

### Example 6

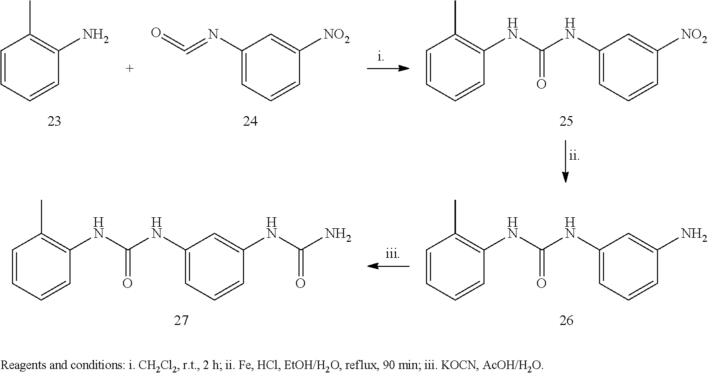

1-(3-nitrophenyl)-3-o-tolylurea (25): 2-amino toluene 23 (300 mg, 300 μL, 2.80 mmol, 1 eq.) was added to a solution of 3-nitrophenyl isocyanate 24 (460 mg, 2.80 mmol, 1 eq.) in anhydrous CH₂Cl₂ (8 mL) in one portion. The solution was stirred for 2 hours at room temperature under a nitrogen atmosphere. The white precipitate was filtered, washed with petroleum ether and dried under high vacuum affording 750 mg (99%) of the desired product as a white solid. Further purification was unnecessary. ¹H NMR (400 MHz, MeOD): δ: 2.31 (s, 3H, CH₃); 7.04-7.07 (t, J=8, 1H), 7.17-7.22 (m, 2H), 7.49-7.53 (t, J=8, 1H); 7.62-7.64 (d, J=8 1H); 7.73-7.75 (d, J=8, 1H); 7.85-7.87 (d, J=8, 1H); 8.50 (s, 1H) Ms (ESI) m/z: 272 [M+H]⁺ Anal. Calcd for (C₁₄H₁₃N₃O₃): C, 61.99; H, 4.83; N, 15.49. Found: C, 62.00; H, 4.88; N, 15.40.

1-(3-aminophenyl)-3-o-tolylurea (26): Iron powder (423 mg, 7.56 mmol, 21 eq.), water (2 mL) and HCl (2 drops) were added consecutively to a solution of 25 (100 mg, 0.36 mmol, 1 eq.) in ethanol (10 mL). After stirring at 95° for 90 min, the reaction mixture was filtered hot. Following an ethanol wash, the filtrates were combined and the solvent removed in vacuo. The crude material was purified by flash chromatography (60:40 ethyl acetate/petroleum ether) to provide 25 (70 mg, 0.29 mmol, 78%) as yellow solid

¹H NMR (400 MHz, MeOD): δ: 2.28 (s, 3H, CH₃); 6.45-6.48 (d, J=8, 1H); 6.69-6.71 (d, J=8, 1H); 6.91 (s, 1H), 7.00-7.03 (m, 2H), 7.15-7.17 (m, 2H), 7.61-7-63 (d, J=8, 1H) Ms (ESI) m/z: 242 [M+H]⁺ Anal. Calcd for (C₁₄H₁₅N₃O): C, 69.69; H, 6.27; N, 17.41. Found: C, 69.64; H, 6.29; N, 17.40.

1-[3-(carbamoylamino)phenyl]-3-(2-methylphenyl)urea (27): The urea derivative 26 (20 mg, 0.08 mmol, 1 eq.) was dissolved in acetic acid concentrated/water (1:1). To this a suspension of potassium cyanate (7 mg, 0.08 mmol, 1 eq.) in warm water was added. The reaction mixture was stirred for 30 min and then it was cooled in ice bath and filtered to obtain the desired product 27 (12 mg, 0.04 mmol, 52%) as white solid. Further purification was unnecessary. ¹H NMR (400 MHz, DMSO): δ 2.15 (s, 3H, CH₃); 5.74 (S, 2H, NH₂); 6.84 (m, 2H); 7.01-7.09 (m, 4H); 7.54 (s, 1H); 7.76-7.79 (m, 2H); 8.44 (s, 1H); 8.94 (s, 1H). Ms (ESI) m/z: 285 [M+H]⁺ Anal. Calcd for (C₁₅H₁₆N₄O₂): C, 63.37; H, 5.67; N, 19.71. Found: C, 63.38; H, 5.60; N, 19.77.

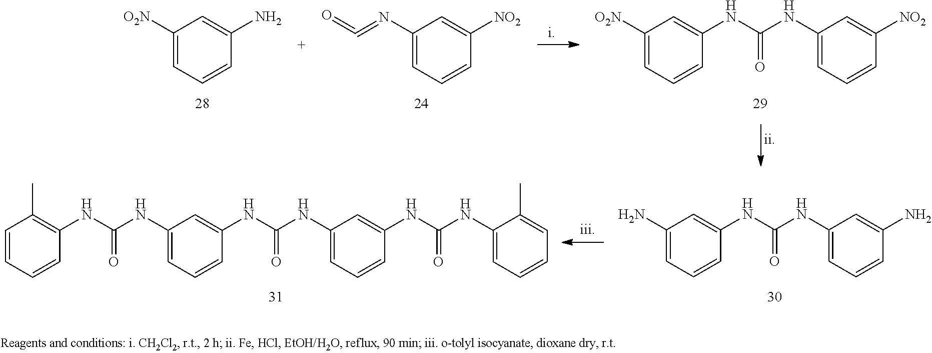

1,3-bis(3-nitrophenyl)urea (29): 3-nitroaniline 28 (300 mg, 2.18 mmol, 1 eq.) was added to a solution of 3-nitrophenyl isocyanate (357 mg, 2.18 mmol, 1 eq.) in anhydrous CH₂Cl₂ (8 mL) in one portion. The solution was stirred for 2 hours at room temperature under a nitrogen atmosphere. The white precipitate was filtered, washed with petroleum ether Drying under high vacuum afforded 550 mg (83%) of the desired product 29 as a white solid. Further purification was unnecessary. ¹H NMR (400 MHz, MeOD): δ: 7.51-7.55 (t, J=8, 2H); 7.78-7.80 (d, J=8, 2H); 7.88-7.90 (d, J=8, 2H); 8.51 (s, 2H); Ms (ESI) m/z: 302 [M+H]⁺ Anal. Calcd for (C₁₃H₁₀N₄O₅): C, 51.66; H, 3.33; N, 18.54. Found: C, 51.69; H, 3.27; N, 18.60.

1,3-bis(3-aminophenyl)urea (30): Iron powder (190 mg, 3.36 mmol, 21 eq.), water (2 mL) and HCl (2 drops) were added consecutively to a solution of 29 (50 mg, 0.16 mmol, 1 eq.) in ethanol (8 mL). After stirring at 95° for 90 min, the reaction mixture was filtered hot. Following an ethanol wash, the filtrates were combined and the solvent removed in vacuo. The crude material was purified by flash chromatography (95:5 ethyl acetate/petroleum ether) to provide 30 (32 mg, 0.13 mmol, 80%) as yellow solid ¹H NMR (400 MHz, MeOD): δ: 6.37-6.41 (t, J=8, 2H); 6.65-6.69 (t, J=8, 2H); 6.86-6.88 (d, J=8, 2H); 6.97-7.02 (q, J=8, 2H); Ms (ESI) m/z: 243 [M+H]⁺ Anal. Calcd for (C₁₃H₁₄N₄O): C, 64.45; H, 5.82; N, 23.13. Found: C, 64.44; H, 5.78; N, 23.18.

1,3-bis(3-{[(2-methylphenyl)carbamoyl]amino}phenyl)urea (31): A solution of o-tolyl-isocyanate (22 mg, 21 μL, 0.16 mmol, 1 eq.) in anhydrous dioxane (1 mL) was slowly added to a solution of 30 (20 mg, 0.08 mmol, 1 eq.) in anhydrous dioxane (1 mL). The reaction mixture was stirred for 24 h at room temperature. The solvent was removed under vacuum; the residual white solid was washed with ethanol, diethyl ether and hexane and filtered. Drying under high vacuum afforded 22 mg (52%) of the desired product 31 as a white solid. Further purification was unnecessary. ¹H NMR (400 MHz, MeOD): δ: 2.16 (s, 6H); 6.84-6.87 (t, J=8, 2H); 6.93-6.95 (d, J=8, 2H); 7.00-7.10 (m, 8H); 7.66 (s, 2H), 7.78 (s, 2H); 8.55 (s, 2H); 8.97 (s, 2H); Ms (ESI) m/z: 509 [M+H]⁺ Anal. Calcd for (C₂₉H₂₈N₆O₃): C, 68.49; H, 5.55; N, 16.52. Found: C, 68.45; H, 5.50; N, 16.54.

### Example 7

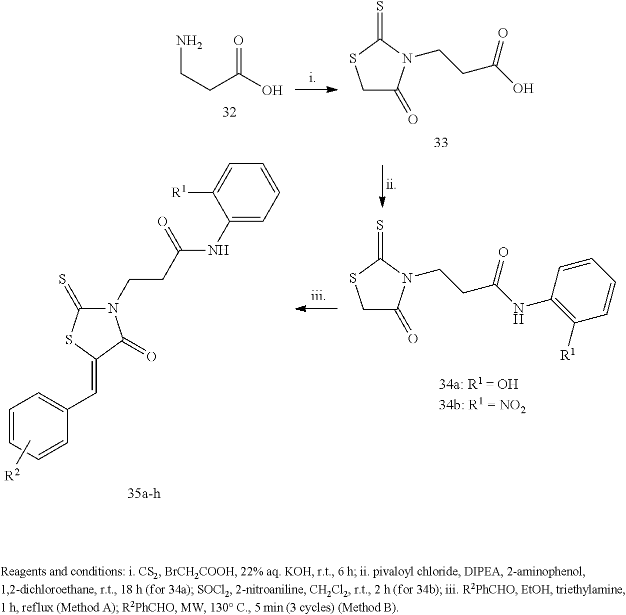

3-(4-Oxo-2-thioxothiazolidin-3-yl)propanoic acid (33). To a solution of β-alanine 32 (3.4 g, 38.1 mmol) in 17 mL of 22% KOH solution, CS₂ 2.5 mL (42 mmol) was added dropwise making sure the temperature of the reaction did not exceed 25° C. The mixture was allowed to stir at r.t. for approximately 3 hours and then bromoacetic acid (5.3 g, 38.1 mmol) was added portionwise over 20 minutes. The reaction mixture was stirred at room temperature for additional 3 hours during which time a precipitate was formed. The pH of the reaction mixture was adjusted to 3-4 using conc. H₂SO₄ and the resulting solution was stirred overnight at room temperature. The precipitate was filtered off and washed with water thus obtaining the desired pure compound 33 (3.52 g, yield 45%) as a yellow solid. Mp 155-156° C. ¹H NMR (400 MHz, MeOD-d₄) δ (ppm) 5.00 (brs, 1H), 4.22 (t, J=7.5, 2H), 4.11 (s, 1H), 2.64 (t, J=7.6, 2H). ¹³C NMR (100 MHz, MeOD-d₄) δ (ppm) 202.58, 174.37, 172.87, 39.64, 35.08, 30.28. MS (ESI) m/z: 204.1 [M−H]⁻. Anal. Calcd for (C₆H₇NO₃S₂): C, 35.11; H, 3.44; N, 6.82. Found: C, 35.10; H, 3.48; N, 6.88.

N-(2-Hydroxyphenyl)-3-(4-oxo-2-thioxothiazolidin-3-yl)propanamide (34a). Compound 33 (1.0 g, 4.87 mmol) and N,N-diisopropylethylamine (932 μL, 5.36 mmol) were dissolved in 20 mL of dry 1,2-dichloroethane and finally pivaloyl chloride (660 μL, 5.36 mmol) was added dropwise. The reaction mixture was stirred at room temperature for 3 hours and then 2-aminophenol (531 mg, 4.87 mmol) was added. The mixture was allowed to stir at room temperature for additional 18 hours. The solvent was removed under reduced pressure and the mixture was purified with flash chromatography (hexane/EtOAc:1/1) to give the desired product 34a (1.233 g, yield 85%) as a yellow solid. Mp 168-170° C. ¹H NMR (400 MHz, DMSO-d₆) δ (ppm) 9.65 (s, 1H), 9.36 (s, 1H), 7.63 (d, J=7.7 1H), 6.95-6.91 (m, 1H) 6.83 (d, J=7.8, 1H) 6.75-6.72 (m, 1H), 4.24 (s, 2H), 4.13 (t, J=7.4, 2H), 2.68 (t, J=7.5, 2H). ¹³C NMR (50 MHz, DMSO-d₆) δ (ppm) 202.75, 173.98, 168.50, 148.10, 125.77, 124.74, 122.85, 118.75, 115.62, 38.2, 35.71, 32.57. MS (ESI) m/z: 295.4 [M−H]⁻. Anal. Calcd for (C₁₂H₁₁N₂O₃S₂): C, 48.63; H, 4.08; N, 9.45. Found: C, 48.66; H, 4.04; N, 9.40.

N-(2-Nitrophenyl)-3-(4-oxo-2-thioxothiazolidin-3-yl)propanamide (34b). To a suspension of 33 (100 mg, 0.48 mmol) in 4 mL of dry dichloromethane, thionyl chloride (40 μL, 0.58 mmol) was added. The reaction was monitored by TLC and after the disappearance of the starting material, 2-nitroaniline (80 mg, 0.58 mmol) was added. The resulting mixture was stirred at room temperature for 2 hours. H₂O was added to the reaction mixture, then it was extracted with EtOAC, dried over Na₂SO₄, concentrated under reduced pressure and purified with flash chromatography (hexane/EtOAc:1/1) to give the desired product 34b (130 mg, yield 84%) as a yellow solid. Mp 170-171° C. ¹H NMR (400 MHz, CDCl₃-d) δ (ppm) 10.27 (s, 1H), 8.64-8.62 (m, 1H), 8.15-8.13 (m, 1H), 7.60-7.56 (m, 1H), 7.15-7.11 (m, 1H), 4.45 (t, J=8, 2H), 3.94 (s, 2H), 2.22 (t, J=8, 2H). ¹³C NMR (100 MHz, DMSO-d₆) δ (ppm) 206.19, 173.99, 168.83, 136.04, 134.16, 128.02, 125.78, 123.59, 122.38, 40.28, 35.43, 34.87. MS (ESI) m/z: 324.4 [M−H]⁻. Anal. Calcd for (C₁₂H₁₁N₃O₄S₂): C, 44.30; H, 3.41; N, 12.91. Found: C, 44.36; H, 3.38; N, 12.90.

### General Procedure for the Synthesis of 35a-h (Method A).

To a solution of the opportune intermediate 34a or 34b (0.33 mmol) in 4 mL of EtOH, the opportune benzaldehyde (0.33 mmol) and triethylamine (0.33 mmol) were added. The reaction mixture was refluxed for 1 hour then the precipitate formed was filtered off and washed with EtOH and hexane to give the pure products.

General procedure for microwave-assisted synthesis of 35a-h (Method B). A mixture of the opportune intermediate 34a or 34b (0.16 mmol) and the appropriate benzaldehyde (0.16 mmol) was irradiated in the microwave for 5 min at 130° C. (3 cycles). The residue was washed with EtOH and hexane to give the desired pure compounds.

(Z)-3-(5-(3-Bromobenzylidene)-4-oxo-2-thioxothiazolidin-3-yl)-N-(2-hydroxy phenyl)propanamide (35a; FE15). Yellow solid. Mp 222-224° C. ¹H NMR (400 MHz, DMSO-d₆) δ (ppm) 9.56 (s, 1H), 9.38 (s, 1H), 7.89 (s, 1H), 7.81 (s, 1H), 7.71 (d, J=7.9 1H), 7.65-7.60 (m, 2H), 7.53-7.49 (m, 1H), 6.92-6.90 (m, 1H) 6.83 (d, J=7.8, 1H) 6.75-6.72 (m, 1H), 4.32 (t, J=7.1, 2H), 2.79 (t, J=7.1, 2H). ¹³C NMR (100 MHz, DMSO-d₆) δ (ppm) 193.37, 169.07, 167.08, 148.66, 135.86, 133.87, 133.85, 132.00, 131.45, 129.04, 126.38, 125.30, 124.72, 123.36, 123.07, 119.33, 116.13, 41.40, 33.39. MS (ESI): m/z 462.9 [M−H]⁺; Anal. Calcd for (C₁₉H₁₅BrN₂O₃S₂): C, 49.25; H, 3.26; N, 6.05. Found: C, 49.31; H, 3.29; N, 6.06.

3-((Z)-5-(3-Fluorobenzylidene)-4-oxo-2-thioxothiazolidin-3-yl)-N-(2-hydroxyphenyl) propanamide (35b). Yield 45%. Yellow solid. Mp 210-211° C. ¹H NMR (400 MHz, acetone-d₆): δ (ppm) 9.17 (s, 1H), 7.69 (s, 1H), 7.59-7.54 (m, 1H), 7.44-7.41 (m, 1H), 7.37 (d, J=8, 1H), 7.26-7.21 (m, 1H), 6.97-6.92 (m, 1H), 6.82 (d, J=8 1H), 6.76-6.72 (m, 1H), 4.45 (t, J=7.1, 2H), 2.92 (t, J=7.1, 2H). ¹³C NMR (75 MHz, DMSO-d₆): δ (ppm) 193.68, 169.30, 167.37, 164.65, 161.39, 148.88, 136.05, 135.94, 132.35, 132.23, 131.96, 126.77, 126.73, 126.60, 126.52, 124.86, 123.59, 119.55, 118.53, 118.24, 118.17, 117.88, 116.34, 40.94, 33.29. MS (ESI) m/z: 401.5 [M−H]⁻. Anal. Calcd for (C₁₉H₁₅FN₂O₃S₂): C, 56.70; H, 3.76; N, 6.96. Found: C, 56.76; H, 3.75; N, 6.98.

3-((Z)-5-(3,5-Difluorobenzylidene)-4-oxo-2-thioxothiazolidin-3-yl)-N-(2-hydroxyphenyl) propanamide (35c). Yield 77%. Yellow solid. Mp 235-237° C. ¹H NMR (400 MHz, DMSO-d₆): δ (ppm) 9.56 (s, 1H), 9.30 (s, 1H), 7.72 (s, 1H), 7.58-7.56 (m, 1H), 7.37-7.33 (m, 1H), 7.28-7.27 (m, 2H), 6.87-6.83 (m, 1H), 6.77-6.75 (m, 1H), 6.68-6.64 (m, 1H), 4.23 (t, J=7.16, 2H), 2.70 (t, J=7.16, 2H). ¹³C NMR (100 MHz, DMSO-d₆): δ (ppm) 193.05, 168.97, 166.96, 164.36, 164.22, 161.89, 161.76, 148.57, 136.72, 130.27, 126.30, 126.11, 125.21, 123.26, 119.25, 116.04, 113.71, 113.44, 106.77, 106.51, 106.25, 41.35, 33.28. MS (ESI) m/z: 419.4 [M−H]⁻. Anal. Calcd for (C₁₉H₁₄F₂N₂O₃S₂): C, 54.28; H, 3.36; N, 6.66. Found: C, 54.30; H, 3.30; N, 6.68.

3-((Z)-5-(3-Methylbenzylidene)-4-oxo-2-thioxothiazolidin-3-yl)-N-(2-hydroxyphenyl) propanamide (35d). Yield 61%. Yellow solid. Mp 208-209° C. ¹H NMR (400 MHz, DMSO-d₆): δ (ppm) 9.57 (s, 1H), 9.30 (s, 1H), 7.69 (s, 1H), 7.57 (d, J=8, 1H), 7.39-7.36 (m, 3H), 7.27-7.25 (m, 1H), 6.87-6.83 (m, 1H), 6.76 (d, J=8, 1H), 6.69-6.64 (m, 1H), 4.25 (t, J=7.2, 2H), 2.72 (t, J=7.2, 2H). ¹³C NMR (100 MHz, DMSO-d₆): δ (ppm) 193.67, 169.01, 167.15, 148.56, 139.31, 133.36, 132.12, 131.43, 129.81, 128.21, 126.32, 125.21, 123.26, 122.64, 119.26, 116.07, 41.22, 33.32, 21.28. MS (ESI) m/z: 397.5 [M−H]⁻. Anal. Calcd for (C₂₀H₁₈N₂O₃S₂): C, 60.28; H, 4.55; N, 7.03. Found: C, 60.21; H, 4.59; N, 7.12.

3-((Z)-5-(3-Methoxybenzylidene)-4-oxo-2-thioxothiazolidin-3-yl)-N-(2-hydroxyphenyl) propanamide (35e). Yield 67%. Yellow solid. Mp 200-202° C. ¹H NMR (400 MHz, DMSO-d₆): δ (ppm) 9.31 (s, 1H), 7.73 (s, 1H), 7.57 (d, J=8, 1H), 7.42-7.38 (m, 1H), 7.14-7.12 (m, 2H), 7.03-7.01 (m, 1H), 6.87-6.83 (m, 1H), 6.76 (d, J=8, 1H), 6.68-6.64 (m, 1H), 4.25 (t, J=7.2, 2H), 2.72 (t, J=7.2, 2H). ¹³C NMR (100 MHz, DMSO-d₆): δ (ppm) 193.68, 169.10, 160.23, 148.68, 134.84, 133.29, 131.13, 126.40, 125.30, 123.37, 122.94, 119.33, 117.47, 116.26, 116.15, 55.83, 41.35, 33.41. MS (ESI) m/z: 413.5 [M−H]⁻. Anal. Calcd for (C₂₀H₁₈N₂O₄S₂): C, 57.95; H, 4.38; N, 6.76. Found: C, 57.97; H, 4.44; N, 6.70.

3-((Z)-5-((Benzo[d][1,3]dioxol-5-yl)methylene)-4-oxo-2-thioxothiazolidin-3-yl)-N-(2-hydroxy phenyl)propanamide (351). Yield 53%. Yellow solid. Mp 240-241° C. ¹H NMR (400 MHz, DMSO-d₆): δ (ppm) 9.56 (s, 1H), 9.30 (s, 1H), 7.66 (s, 1H), 7.58-7.56 (m, 1H), 7.14-7-12 (m, 1H), 7.09 (s, 1H), 7.04-7.01 (m, 1H), 6.87-6.84 (m, 1H), 6.77-6.75 (m, 1H), 6.68-6.65 (m, 1H), 6.07 (s, 2H), 4.23 (t, J=7.2, 2H), 2.70 (t, J=7.2, 2H). ¹³C NMR (100 MHz, DMSO-d₆): δ (ppm) 193.29, 169.03, 167.18, 150.29, 148.77, 148.57, 133.53, 127.61, 127.40, 126.32, 125.20, 123.26, 120.18, 119.26, 116.07, 110.01, 109.74, 102.60, 41.19, 33.32. MS (ESI) m/z: 427.0 [M−H]⁻. Anal. Calcd for (C₂₀H₁₆N₂O₅S₂): C, 56.06; H, 3.76; N, 6.54. Found: C, 56.03; H, 3.84; N, 6.51.

3-((Z)-5-(3-Bromobenzylidene)-4-oxo-2-thioxothiazolidin-3-yl)-N-(2-nitrophenyl)propanamide (35g). Yield 85%. Yellow solid. Mp 204-206° C. ¹H NMR (400 MHz, DMSO-d₆): δ (ppm) 10.32 (s, 1H), 7.87-7.84 (m, 1H), 7.79 (s, 1H), 7.72 (s, 1H), 7.63-7.60 (m, 2H), 7.54-7.52 (m, 2H), 7.45-7.41 (m, 1H), 7.31-7.27 (m, 1H), 4.23 (t, J=7.4, 2H), 2.70 (t, J=7.4, 2H). ¹³C NMR (100 MHz, DMSO-d₆): δ (ppm) 193.43, 168.87, 166.99, 142.99, 135.76, 134.32, 133.78, 131.93, 131.39, 131.18, 128.95, 126.00, 125.84, 125.24, 124.66, 123.00, 40.86, 33.20. MS (ESI) m/z: 491.4 [M−H]⁻. Anal. Calcd for (C₁₉H₁₄BrN₃O₄S₂): C, 46.35; H, 2.87; N, 8.53. Found: C, 46.42; H, 2.85; N, 8.56.

3-((Z)-5-(3-Fluorobenzylidene)-4-oxo-2-thioxothiazolidin-3-yl)-N-(2-nitrophenyl)propanamide (35h). Yield 77%. Yellow solid. Mp 207-208° C. ¹H NMR (400 MHz, DMSO-d₆): δ (ppm) 10.32 (s, 1H), 7.87-7.85 (m, 1H), 7.74 (s, 1H), 7.64-7.60 (m, 1H), 7.54-7.52 (m, 2H), 7.45-7.42 (m, 1H), 7.39-7.37 (m, 1H), 7.31-7.27 (m, 2H), 4.23 (t, J=8, 2H), 2.70 (t, J=8, 2H). ¹³C NMR (100 MHz, DMSO-d₆): δ (ppm) 193.61, 168.95, 167.14, 164.03, 161.58, 143.09, 135.81, 135.73, 134.39, 132.11, 132.03, 131.75, 131.26, 126.52, 126.08, 125.92, 125.32, 124.68, 118.27, 118.06, 117.90, 117.67, 40.94, 33.29. MS (ESI) m/z: 430.5 [M−H]⁻. Anal. Calcd for (C₁₉H₁₄FN₃O₄S₂): C, 52.89; H, 3.27; N, 9.74. Found: C, 52.80; H, 3.26; N, 9.81.

### Example 8

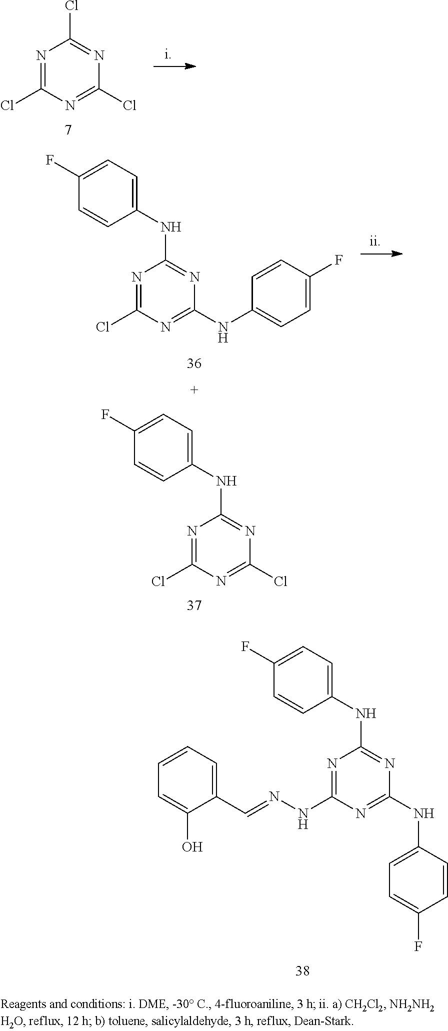

6-Chloro-N2,N4-bis(4-fluorophenyl)-1,3,5-triazine-2,4-diamine (36): To a stirred solution of cyanuric chloride 7 (2 g, 10.86 mmol) in dimethoxyethane (20 mL) at −30° C., 4-fluoroaniline (1 eq) was added dropwise. The reaction mixture was vigorously stirred for 3 h at −30° C. then warmed up to r.t. and washed with 3N HCl, water, brine and finally dried over anhydrous Na₂SO₄. The organic phase was evaporated to dryness and the white residue was purified by flash chromatography (petroleum ether/Et₂O 3/1) to give the desired product 36 and the side product 37. Yield 50%. ¹H NMR (400 MHz, CDCl₃-d): δ (ppm) 7.79 (brs, 2H), 7.39-7.36 (m, 4H), 7.04-7.02 (m, 4H). MS (ESI) m/z: 30: 334.7 [M+H]⁺;]⁺. Anal. Calcd for (C₁₅H₁₀ClF₂N₅): C, 53.99; H, 3.02; N, 20.99. Found: C, 53.94; H, 3.05; N, 20.95.

4,6-Dichloro-N-(4-fluorophenyl)-1,3,5-triazin-2-amine (37). Yield 35%; Mp 172° C.; ¹H NMR (400 MHz, CDCl₃-d): δ (ppm) 7.79 (brs, 1H), 7.41 (q, J=8.60, 2H), 7.03 (t, J=8.63, 2H). ¹³C NMR (100 MHz, CDCl₃-d): δ (ppm) 171.28, 164.33, 161.68, 159.23, 131.62, 123.80, 116.26. MS (ESI) m/z: 260.1 [M+H]⁺; 282.1 [M+Na]⁺. Anal. Calcd for (C₉H₅Cl₂FN₄): C, 41.73; H, 1.95; N, 21.63. Found: C, 41.77; H, 1.93; N, 21.64.

(E)-2-((2-(4,6-bis(4-Fluorophenylamino)-1,3,5-triazin-2-yl)hydrazono)methyl)phenol (38): To a solution of 36 (1 eq) in dichloromethane, hydrazine (4 eq) was added and the resulting mixture was refluxed for 12 h. After cooling down to r.t., the mixture was washed with water, brine and finally dried over anhydrous Na₂SO₄. The organic phase was evaporated to dryness, the residue was dissolved in toluene and reacted with salicylaldehyde (2 eq). The reaction mixture was refluxed for 3 h using a Dean-Stark apparatus for azeotropical removal of water and then evaporated to dryness. The resulting residue was dissolved in the minimum amount of dichloromethane; by addition of petroleum ether the desired final compounds were collected by filtration.

Yield 48%. ¹H NMR (400 MHz, CDCl₃-d): δ (ppm) 11.20 (brs, 1H), 7.80 (s, 1H), 7.40 (brs, 3H), 7.22-7.19 (m, 2H), 7.04-7.02 (m, 1H), 6.96-6.94 (m, 4H), 6.82-6.78 (m, 1H). MS (ESI) m/z: 434.4 [M+H]⁺. Anal. Calcd for (C₂₂H₁₇F₂N₇O): C, 60.97; H, 3.95; N, 22.62. Found: C, 60.94; H, 3.96; N, 22.55.

### Example 9

### Anti-Enzymatic Activity:

The anti-enzymatic activity of representative compounds of the invention against the DDX3 ATPase activity is reported in Table 1.

TABLE 1
Activity of representative compounds of the invention against DDX3 ATPase

| Compound ID^{a} | Structure | Ki (μM)^{b} |
|---|---|---|
| FE-15 (35a) | 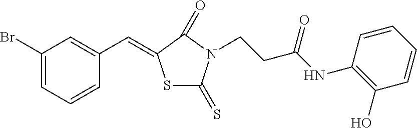 | 4.5 |
| FE-56 | 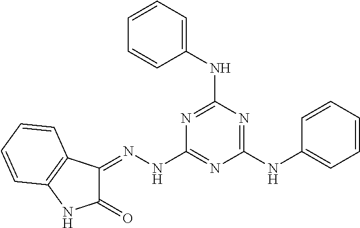 | 0.4 |
| FE-56A | 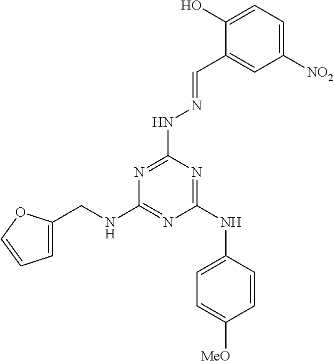 | 31.7 |
| FE-56F | 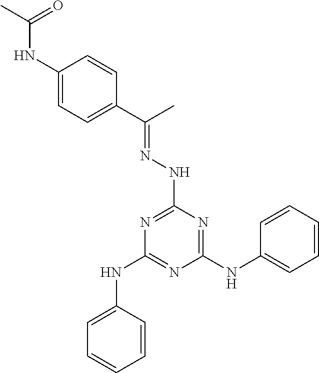 | 0.3 |
| FE-56I | 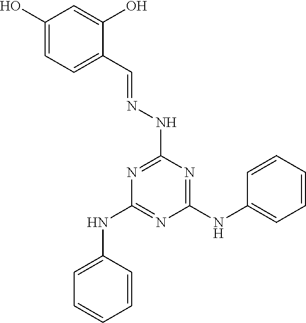 | 33 |
| FE-56M | 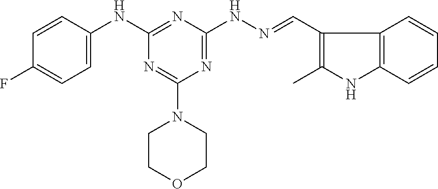 | 0.2 |
| FE-56N | 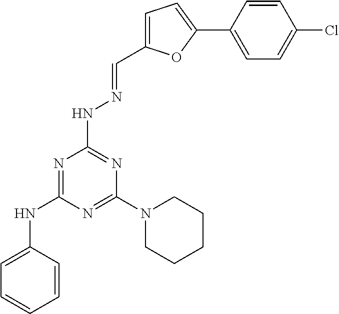 | 0.4 |
| FE-56O | 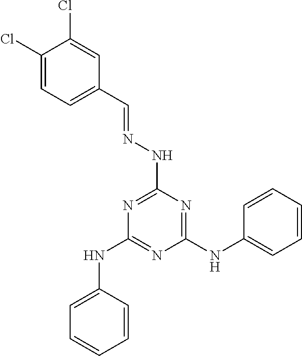 | 18 |
| FE-56U | 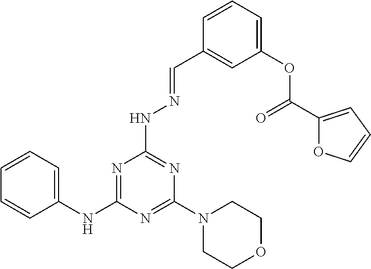 | 6.3 |
| FE-56V | 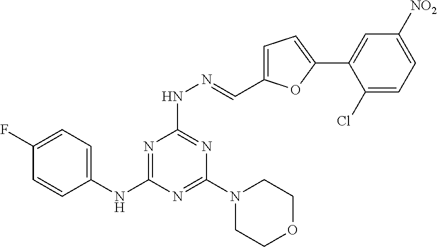 | 3.6 |
| FE-56Z | 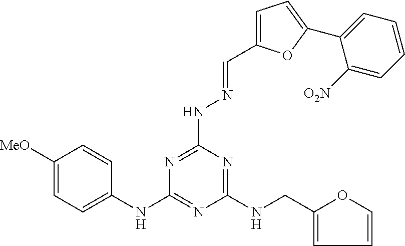 | 1.9 |
| FE-56AA | 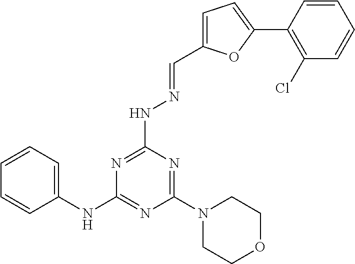 | 3.2 |
| FE-56AD | 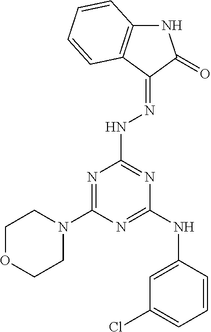 | 2 |
| FE-56AH | 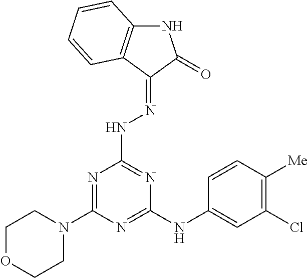 | 3.2 |
| FE-56AN | 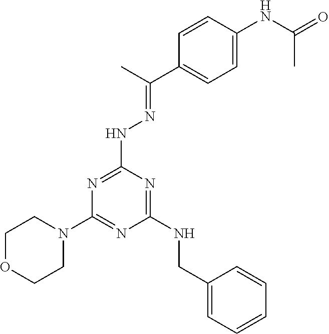 | 0.5 |
| FE-56AI | 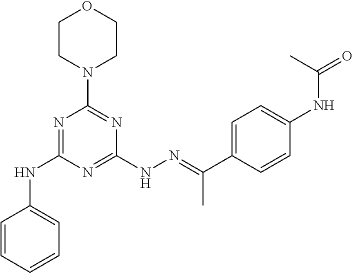 | 2.2 |
| FE-56AM | 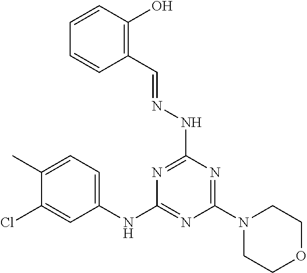 | 1.6 |
| 11a | 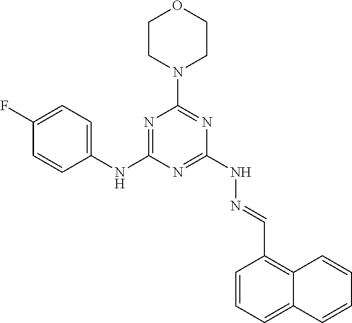 | 86 |
| 11b | 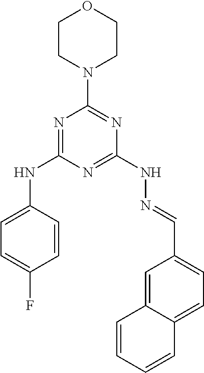 | 8.5 |
| 11c | 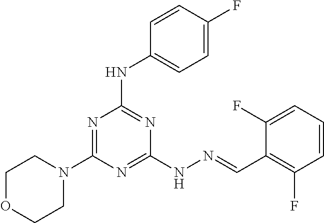 | 2.6 |
| 11d | 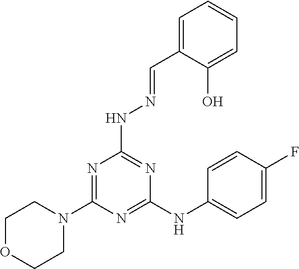 | 0.4 |
| 14f | 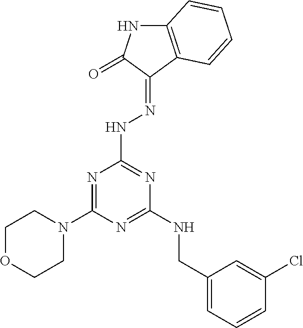 | 2.9 |
| 14g | 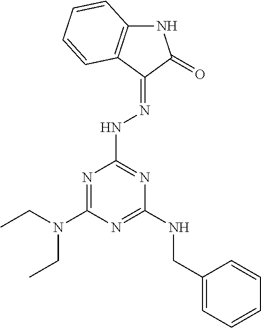 | 0.1 |
| 15h | 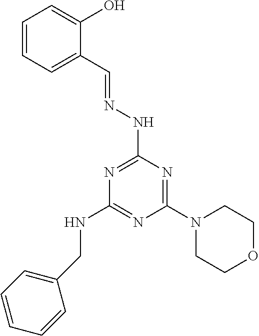 | 0.6 |
| 15j | 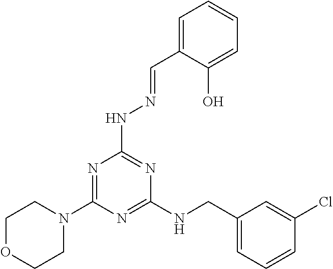 | 62 |
| 15k | 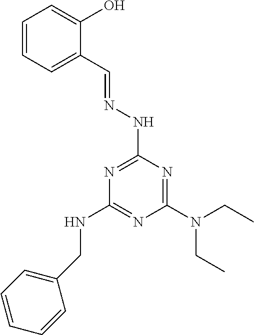 | 0.5 |
| 38 | 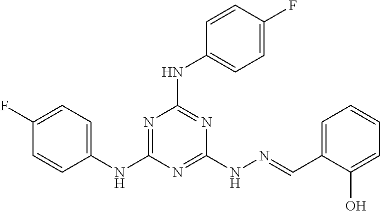 | 0.1 |
| 35b | 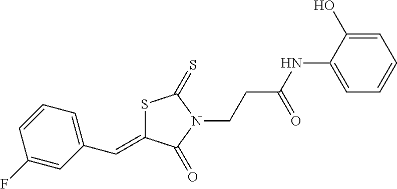 | 0.3 |
| 35c | 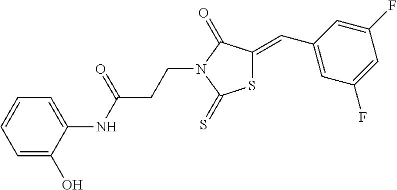 | 0.5 |
| 35d | 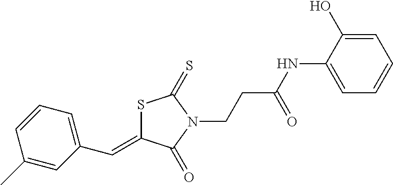 | 97 |
| 35e | 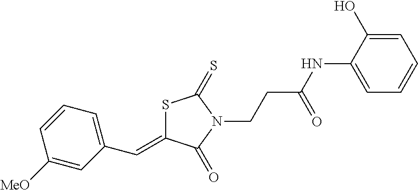 | 10 |
| 35f | 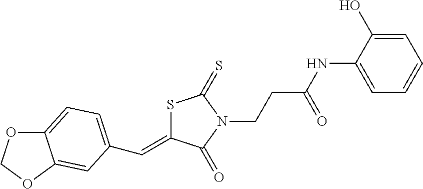 | 1.0 |
| 35g | 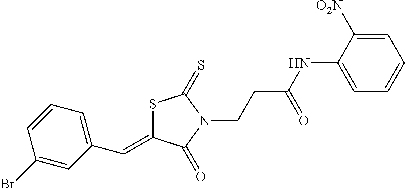 | 8.7 |
| 35h | 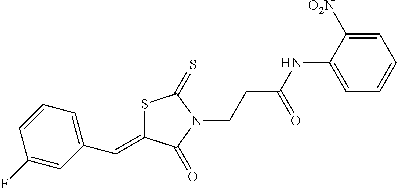 | 7.6 |
| 3j | 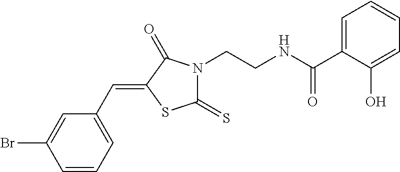 | 4.2 |
| 3k | 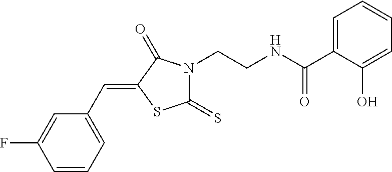 | 4.3 |
| 6b | 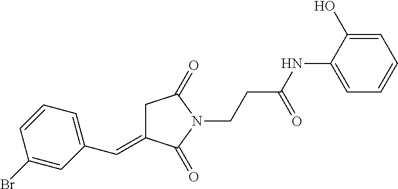 | 28 |
| FE-70 | 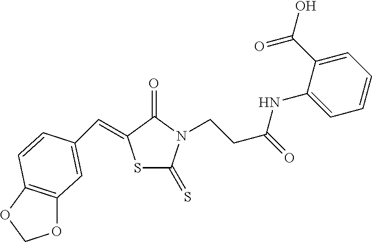 | 0.4 |
| FE-77 | 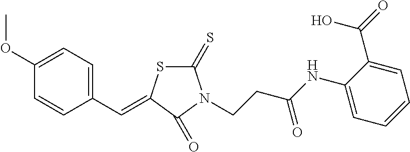 | 2.0 |
| FE-69 | 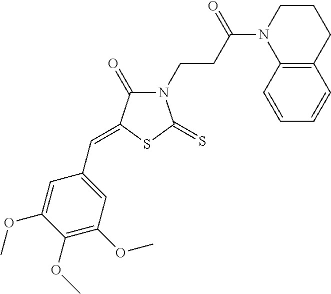 | 0.9 |
| FE-74 | 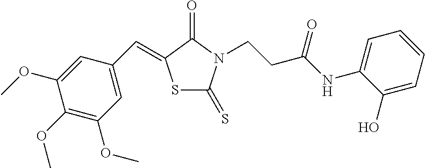 | 0.1 |
| 22 | 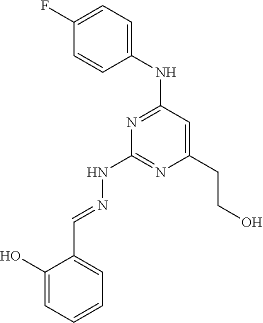 | 9.0 |

^{a}FE-compounds were obatained by commercial sources (Asinex and ChemBridge) while the other were synthesized using the procedures reported in the examples 1-5 and 7-8.
^{b}Apparent equilibrium dissociation constant for the inhibitor to the target enzyme DDX3, expressed in micromolar concentration.

### Example 10

### Antiviral Activity:

Selected compounds from Table 1 were tested against HIV-1 replication in two different model systems. FIG. 1A shows the effects of compounds FE56 and 35b against HIV-1 single-round replication in HeLaCD4+ cells infected with a wild type HIV-1 strain. The data clearly show a dose-dependent reduction of viral load (quantified as viral RNA copies), indicative of an inhibition of viral replication. FIG. 1 B shows the effects of compounds FE15 and 35b against multiple rounds of replication of wild type HIV-1 in natural target cells (PBMC). The data clearly show a significant reduction of viral RNA at 5 days post infection. Together, the data presented in FIG. 1 clearly demonstrate that selected compounds of the invention are capable to inhibit HIV-1 replication in two different model systems.

It is interesting to note that the inhibitor FE15 displayed very low cytotoxicity on HeLa cells (CC₅₀>200 μM) but only modest anti-HIV activity (EC₅₀=83 μM).

FIG. 2A shows the antiviral activity of FE15 on PBMCs infected with HIV-1, in comparison with the most potent derivatives 35b, 11d and 38. It can be appreciated that the compounds 11d and 38 showed between 40% and 60% reduction of viral load already at 3 μM, while FE15 needed to be added at 50 μM to show comparable effects. Dose response curves were generated for FE15 and 38. As shown in FIG. 2B, 38 showed a 41-fold higher antiviral potency on HIV-1 infected PBMCs (EC₅₀=2 μM) than the inhibitor FE15 (EC₅₀=83 μM). Similar curves yielded EC₅₀ values for 35b and 11d of 7.5 and 2.5 μM, respectively. The antiviral potencies and toxicity indexes of the most active compounds are summarized in the Table 2 below.

TABLE 2
Antiviral and cytotoxic activity of selected compounds

| | Antiviral | Cytotoxicity (CC₅₀)^{b} | | | Selectivity index^{c} | | |
|---|---|---|---|---|---|---|---|
| Comd. | activity^{a} | HeLa | MOLT-4 | PBMCs | HeLa | MOLT-4 | PBMCs |
| FE15 | 83 ± 7 | >200 | >200 | >150 | 2.4 | 2.4 | 1.8 |
| (35a) | | | | | | | |
| FE66 | 7.5 ± 0.7 | 50 ± 5 | 50 ± 10 | 50 ± 5 | 6.7 | 6.7 | 6.7 |
| (35b) | | | | | | | |
| 16c | 2.5 ± 0.1 | 2.5 ± 0.3 | 5 ± 1 | 10 ± 1 | 1.0 | 2.0 | 4.0 |
| 38 | 2.0 ± 0.2 | 20 ± 2 | 10 ± 1 | 8.0 ± 0.5 | 10 | 5.0 | 4.0 |

^{a}dose required to reduce 50% viral proliferation (measured as viral RNA load)
^{b}dose requried to reduce 50% cell vitality
^{c}Expressed as CC₅₀/EC₅₀

The data shown in Table 2, clearly demonstrate the ability of the compounds of the invention to potently suppress cellular proliferation of the highly proliferating HeLa cancer cell line.

### Example 11

Antiproliferative Activity:

Selected compounds from Table 1 were tested against the cervical cancer HeLa cell line for antiproliferative activity. Cell viability was measured with the CellTiter viability assay (Table 2). Table 3 lists the calculated antiproliferative potencies (EC₅₀, dose required to suppress 50% of cell viability) for a series of representative compounds of the invention.

TABLE 3
EC50 values for antiproliferative activity against HeLa cells

| | | EC50 (μM) | EC50 (μM) |
|---|---|---|---|
| Compound ID^{a} | Structure | 48 hours | 72 hours |
| 35b | 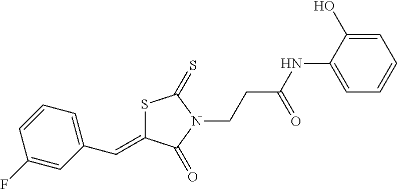 | 50 | 10 |
| FE-56A | 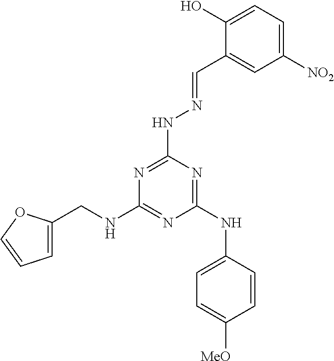 | 8 | 4 |
| FE-56F | 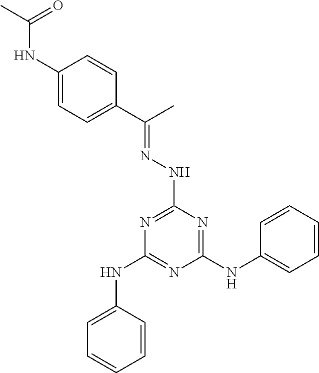 | 10 | 5 |
| FE-56M | 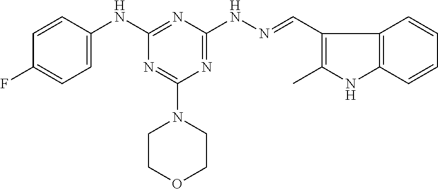 | 10 | 5 |
| FE-56N | 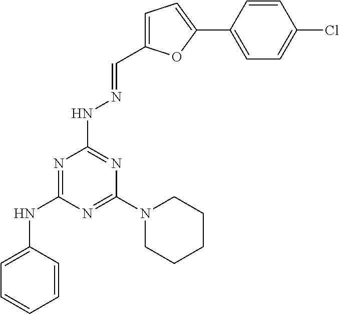 | 25 | 4 |
| 15k | 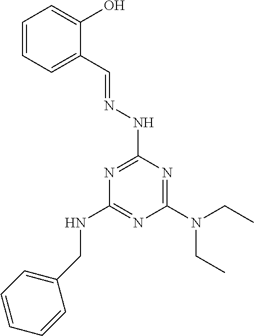 | 3 | 1 |
| 15h | 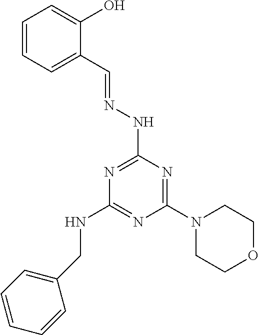 | 5 | 2 |
| 14g | 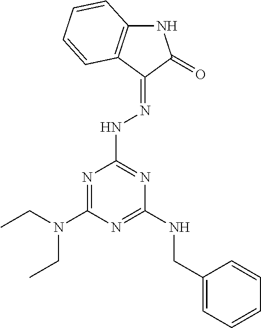 | 10 | 10 |

^{a}FE-compounds were obatained by commercial sources (Asinex and ChemBridge) while the other were synthesized using the procedures reported in the examples 4 and 7.

### Example 12

### Anti-Helicase Activity

Compounds were obtained by application of a virtual docking protocol on the RNA-binding site identified in the homology model developed in the present invention. These compounds were commercially available (EI-compounds in Table 4). A few other compounds (25-31, EXAMPLE 6) were rationally designed to target the above mentioned RNA-binding site and then synthesized. All the compounds were tested for anti-helicase activity. The results are reported in Table 4.

TABLE 4
anti-helicase and ATPase activity of the compound of the invention

| | | Helicase | ATPase |
|---|---|---|---|
| | | ID50 | ID50 |
| Compound ID^{a} | Structure | (μM) | (μM) |
| EI-01 | 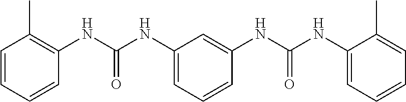 | 250 | 245 |
| EI-01A | 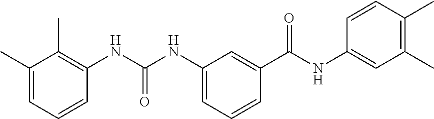 | 131 | |
| EI-01B | 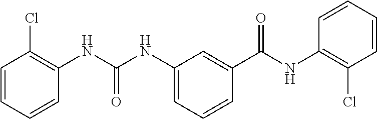 | 1300 | |
| 25 | 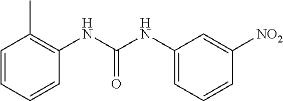 | 1 | 11 |
| 26 | 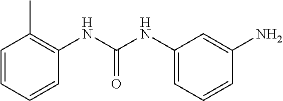 | 5.7 | |
| 27 | 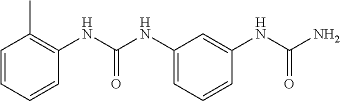 | na^{b} | |
| 29 | 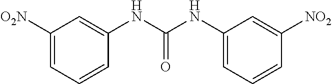 | 52 | |
| 30 | 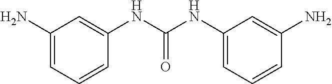 | 24 | |
| 31 | 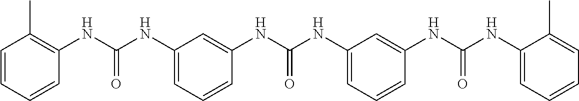 | 1300 | |

^{a}EI-compounds were obtained by commercial sources (Asinex and ChemBridge) while the other were synthesized using the procedures reported in the example 6.
^{b}na = not active

Among those of the first series, compound EI-01 proved to be the most potent, but did not discriminate between ATPase and helicase activities. Compounds 25 and 26 showed 245- and 43-fold improvement in their anti-helicase activity, with respect to EI01. Moreover, compound 25 showed an 11-fold higher potency against the helicase, with respect to the ATPase activity.

### Results

The anti-DDX3 activity of the compounds of the invention was evaluated by in vitro ATPase activity assay using the recombinant human DDX3 protein produced in E. coli. The potency of inhibition (Ki) for each molecule was calculated, representing the concentration of the inhibitor able to reduce the DDX3 ATPase activity by 50%

The most active compounds were further optimized and tested in anti-HIV and antiproliferative cellular assays.

The results obtained and reported in the Examples show that the compounds of the invention were able to:

1) inhibit the ATPase activity of the human DDX3 protein by interacting with the ATP binding site and interfering with the subsequent catalytic steps through a mixed-type inhibitory mechanism;
2) suppress HIV-1 replication in infected cells with low toxicity to uninfected control cells;
3) suppress proliferation of the HeLa cervical cancer tumor cell line.

The data of the present invention demonstrate the capacity of the present compounds to inhibit proliferation of HIV-1 as well as cancer cells. For these reasons, such molecules, used alone or in synergy (cocktail), are suitable for the treatment of viral (e.g. HIV-1 and HCV) infections and for the treatment of diseases caused by uncontrolled cellular proliferation (cancer).

### REFERENCES

1. Arkin M R, Wells J A. Small-molecule inhibitors of protein-protein interactions: progressing towards the dream. Nat Rev Drug Discov. 2004 April; 3(4):301-17
2. Armand-Ugon M, Clotet-Codina I, Tintori C, Manetti F, Clotet B, Botta M, Este J A. The anti-HIV activity of ADS-J1 targets the HIV-1 gp120. Virology. 2005 Sep. 13
3. Bieniasz P. D., and B. R. Cullen. 2000. Multiple blocks to human immunodeficiency virus type 1 replication in rodent cells. J. Virol. 74:9868-9877.
4. Botlagunta M, Vesuna F, Mironchik Y, Raman A, Lisok A, Winnard Jr P, Mukadam S, Van Diest P, Chen J H, Farabaugh P, Patel A H and Raman V (2008). Oncogenic role of DDX3 in breast cancer biogenesis. Oncogene, 27: 3912-3922.
5. Chao C H, Chen C M, Cheng P L, Shih J W, Tsou A P and Lee Y H (2006). DDX3, a DEAD box RNA helicase with tumor growth-suppressive property and trancriptional regulation activity of the p21 waf1/cip1 promoter, is a candidate tumor suppressor. Cancer Res., 66: 6579-6588.
6. Cherepanov, P., Maertens, G., Proost, P., Devreese, B., Van Beeumen, J., Engelborghs, Y., De Clercq, E., and Debyser, Z. (2003) HIV-1 integrase forms stable tetramers and associates with LEDGF/p75 protein in human cells. J. Biol. Chem. 278, 38372-38381.
7. Cohen, J. 2001. Building a small-animal model for AIDS, block by block. Science. 293:1034-6.
8. Cona, A., Manetti, F., Leone, R., Corelli, F., Tavladoraki, P., Polticelli, F., Botta, M. Molecular Basis for the Binding of Competitive Inhibitors of Maize Polyamine Oxidase. Biochemistry 2004, 43, 3426-3435
9. Cullen B R. Nuclear mRNA export: insights from virology. Trends Biochem Sci. 2003 August; 28(8):419-24
10. Elbashir et al., 2001
11. Farnet C M, Bushman F D. HIV-1 cDNA integration: requirement of HMG I(Y) protein for function of preintegration complexes in vitro. Cell. 1997 Feb. 21; 88(4):483-92
12. Hannon G J, Rossi J J. Unlocking the potential of the human genome with RNA interference. Nature. 2004 Sep. 16; 431(7006):371-8
13. Heger P, Lohmaier J, Schneider G, Schweimer K, Stauber R H. Qualitative highly divergent nuclear export signals can regulate export by the competition for transport cofactors in vivo. Traffic. 2001 August; 2(8):544-55
14. Huang J S, Chao C C, Su T L, Yeh S H, Chen D S, Chen C T, Chen P J and Jou Y S (2004). Diverse cellular transformation capability of overexpressed genes in human hepatocellular carcinoma. Biochem. Biophys. Res. Commun., 315: 950-958.
15. Kalpana G V, Marmon S, Wang W, Crabtree G R, Goff S P. Binding and stimulation of HIV-1 integrase by a human homolog of yeast transcription factor SNF5. Science. 1994 Dec. 23; 266(5193):2002-6
16. Kau T R, Way J C, Silver P A. Nuclear transport and cancer: from mechanism to intervention. Nat Rev Cancer. 2004 February; 4(2):106-17
17. Keppler, O. T., W. Yonemoto, F. J. Welte, K. S. Patton, D. Iacovides, R. E. Atchison, T. Ngo, D. L. Hirschberg, R. F. Speck, and M. A. Goldsmith. 2001. Susceptibility of rat-derived cells to replication by human immunoficiency virus type 1. J. Virol. 75:8063-8073.
18. Keppler, O. T., F. J. Welte, T. A. Ngo, P. Chin, K. S. Patton, C.-T. Tsou, N. W. Abbey, M. E. Sharkey, R. M. Grant, Y. You, J. D. Scarborough, W. Ellmeier, D. R. Littman, M. Stevenson, I. F. Charo, B. G. Herndier, R. F. Speck, and M. A. Goldsmith. 2002. Progress toward a human CD4/CCR5-transgenic rat model for de novo infection by human immunodeficiency virus type 1. J. Exp. Med. 195:719-736.
19. Keppler, O. T., I. Allespach, L. Schuller, D. Fenard, W. C. Greene, and O. T. Fackler. 2005. Rodent cells support key functions of the human immunodeficiency virus type 1 pathogenicity factor Nef. J. Virol. 79:1655-65.
20. Knauer, S. K., and R. H. Stauber. (2005) Development of an autofluorescent translocation biosensor system to investigate protein-protein interactions in living cells. Anal Chem. 77 (4815-4820).
21. Knauer, S., Berg, T., Liebel, U., Pepperkok, R. and Stauber, R. H. (2005). Translocation Biosensors To Study Signal Specific Nucleo-Cytoplasmic Transport, Protease Activity & Protein Interactions. Traffic 6 (594-606).
22. Lipinski C, Hopkins A. Navigating chemical space for biology and medicine. Nature. 2004 Dec. 16; 432(7019):855-61
23. Maertens G, Vercammen J, Debyser Z, Engelborghs Y. Measuring protein-protein interactions inside living cells using single color fluorescence correlation spectroscopy. Application to human immunodeficiency virus type 1 integrase and LEDGF/p75. FASEB J. 2005 June; 19(8):1039-41
24. Manetti, F., Esté, J. A., Clotet-Codina, I., Armand-Ugón, M., Maga, G., Crespan, E., Cancio, R., Mugnaini, C., Bernardini, C., Togninelli, A., Carmi, C., Alongi, M., Petricci, E., Massa, S., Corelli, F., Botta, M. Parallel Solution-phase and Microwave-assisted Synthesis of New S-DABO Derivatives Endowed with Subnanomolar Anti-HIV-1 Activity. J. Med. Chem. 2005, 48, 8000-8008.
25. Manetti, F., Schenone, S., Bondavalli, F., Brullo, C., Bruno, O., Ranise, A., Mosti, L., Menozzi, G., Fossa, P., Trincavelli, M. L., Martini, C., Martinelli, A., Tintori, C., Botta, M. Synthesis and 3D QSAR of new pyrazolo[3,4-b]pyridines, potent and selective inhibitors of A₁ adenosine receptors. J. Med. Chem. DOI: 10.1021/jm050407k
26. Mariani, R., G. Rutter, M. E. Harris, T. J. Hope, H. G. Kräusslich, and N. R. Landau. 2000. A block to human immunodeficiency virus type 1 assembly in murine cells. J. Virol. 74:3859-3870
27. Martinez M A, Gutierrez A, Armand-Ugon M, Blanco J, Parera M, Gomez J, Clotet B, Este J A. Suppression of chemokine receptor expression by RNA interference allows for inhibition of HIV-1 replication. AIDS. 2002 Dec. 6; 16(18):2385-90
28. Novina C D, Murray M F, Dykxhoorn D M, Beresford P J, Riess J, Lee S K, Collman R G, Lieberman J, Shankar P, Sharp P A. siRNA-directed inhibition of HIV-1 infection. Nat Med. 2002 July; 8(7):681-6.
29. Owsianka A M and patel A H (1999). Hepatitis C virus core protein interacts with a human DEAD box protein DDX3. Virology, 257: 330-340.
30. Pagliaro L, Felding J, Audouze K, Nielsen S J, Terry R B, Krog-Jensen C, Butcher S. Emerging classes of protein-protein interaction inhibitors and new tools for their development. Curr Opin Chem. Biol. 2004 August; 8(4):442-9
31. Pante N. Nuclear pore complex structure: unplugged and dynamic pores. Dev Cell. 2004 December; 7(6):780-1
32. Pemberton L F, Paschal B M. Mechanisms of receptor-mediated nuclear import and nuclear export. Traffic. 2005 March; 6(3):187-98.
33. Qin X F, An D S, Chen I S, Baltimore D. Inhibiting HIV-1 infection in human T cells by lentiviral-mediated delivery of small interfering RNA against CCR5. Proc Natl Acad Sci USA. 2003 Jan. 7; 100(1):183-8
34. Sticht J, Humbert M, Findlow S, Bodem J, Müller B, Dietrich U, Werner J, Kräusslich H G. A peptide inhibitor of HIV-1 assembly in vitro. Nature Struct Mol Biol, 2005, doi:10.1038/nsmb964.
35. Trono, D., and Baltimore, D. (1990) A human cell factor is essential for HIV-1 Rev action. EMBO J. 9, 4155-4160
36. Vandekerckhove, L., Christ, F., Van Maele, B., De Rijck, J., Gijsbers, R., Van den Haute, C., Witvrouw, M. and Debyser, Z. Transient and stable knock-down of the integrase co-factor LEDGF/p75 reveals its role in the replication cycle of the Human Immunodeficiency Virus. J. Virol. 2005. In Press.
37. von Schwedler, U. K., M. Stuchell, B. Muller, D. M. Ward, H. Y. Chung, E. Morita, H. E. Wang, T. Davis, G. P. He, D. M. Cimbora, A. Scott, H. G. Krausslich, J. Kaplan, S. G. Morham, and W. I. Sundquist. 2003. The protein network of HIV budding. Cell. 114:701-13.
38. Wei, P., M. E. Garber, S. M. Fang, W. H. Fischer, and K. A. Jones. 1998. A novel CDK9-associated C-type cyclin interacts directly with HIV-1 Tat and mediates its high-affinity, loop-specific binding to TAR RNA. Cell. 92:451-462.
39. Yedavalli, V. S., C. Neuveut, Y. H. Chi, L. Kleiman, and K. T. Jeang. 2004. Requirement of DDX3 DEAD box RNA helicase for HIV-1 Rev-RRE export function. Cell. 119:381-92.
40. Zheng, Y. H., H. F. Yu, and B. M. Peterlin. 2003. Human p32 protein relieves a post-transcriptional block to HIV replication in murine cells. Nat Cell Biol. 5:611-8.
41. Maga, G.; Falchi, F.; Garbelli; Belfiore, A.; Witvrouw, M.; Manetti, F.; Botta, M. Pharmacophore Modeling and Molecular Docking Led to the Discovery of Inhibitors of Human Immunodeficiency Virus-1 Replication Targeting the Human Cellular Aspartic Acid-Glutamic Acid-Alanine-Aspartic Acid Box Polypeptide 3. J. Med. Chem. 2008, 51, 6335-6338.

1. A compound of formula 1, 2, 3 or 4: 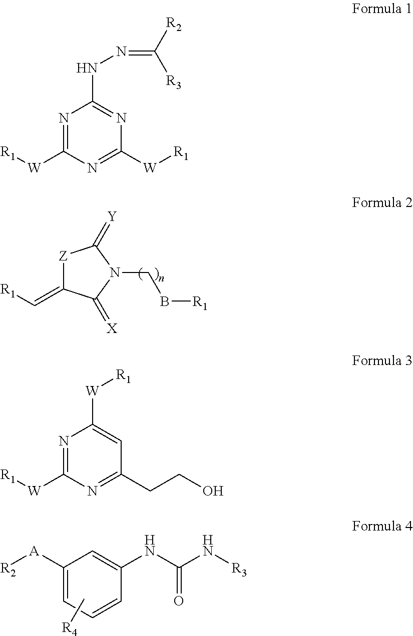
wherein:
Z represents CH₂ or S;
X and Y represent independently O or S;
n is comprised between 0 and 4;
B is absent or represents 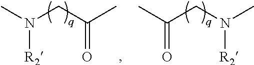
q is comprised between 0 and 2 and R₂′ represents H, —(CH₂)_{w′}—OH, or —(CH₂)_{w′}—NH₂, w′ is an integer from 1 to 3; B is also C═O;
R₁, R₂, and R₃ are each independently selected from the group consisting of: H, a linear or branched alkyl group comprising 1 to 6 carbon atoms, unsubstituted or substituted phenyl radical, an unsubstituted or substituted phenylalkenyl radical, an unsubstituted or substituted phenyalkynyl radical, an unsubstituted or substituted biphenylalkyl radical, an unsubstituted or substituted heterocyclic radical, an unsubstituted or substituted polycyclic radical, an unsubstituted or substituted alicyclic radical or a radical of the formula (R₁a-)ₘ(L-)ₚR₁b-, wherein R₁a and R₁b may be the same or different and represent an unsubstituted or substituted heterocyclic radical or an unsubstituted or substituted phenyl radical, R₁a also represents an unsubstitutetd or substituted polycyclic radical and L represents a divalent linking moiety selected from the group consisting of a valence bond, —(CH₂)_{q}—, —HC═CH—, —C≡C—, —C(═O)—, —O—, —S—, —S(═O)—, —S(═O)₂—, —NHCONH— or NR₁c, R₁c being hydrogen or alkyl, m and p are each independently 0 or 1, and q is an integer from 1 to 3;
R₂ and R₃ together optionally form a cycloalkyl, cycloalkenyl, non-aromatic heterocyclic, or fused or polycyclic ring, 2-oxindole wherein said cycloalkyl, cycloalkenyl, non-aromatic heterocyclic and fused or polycyclic ring are optionally substituted with one or more substituents selected from the groups above described;
wherein the heterocyclic radical is selected from the group consisting of morpholine, thiomorpholine, piperidine, piperazine, pyrrolidine, furan, thiophene, oxazole, oxadiazole, isoxazole, pyridine, 1,3-oxathiolane, thiazole, isothiazole, thiadiazole, imidazole, pyrrole, tetrazole or triazine;
wherein the polycyclic radical is selected from the group consisting benzofuran, isobenzofuran, benzothiophene, isobenzothiophene, benzoxazole, indole, 2-isoindole, 2-oxindole, 2-methylindole, benzopyrazole, quinoline, isoquinoline, tetrahydroquinoline, 1,3-benzodioxole, 1,2-benzodiazine, 1,3-benzodiazine, 1,2,3-benzotriazole, benzothiazole, benzimidazole, 1,2,3-benzotriazine, 1,2,4-benzotriazine, naphtalene, antracene or fluorene;
wherein the alicyclic radical preferably comprises 5 to 8 carbon atoms;
wherein the heterocyclic radical substituents, the polycyclic radical substituents and the alicyclic radical substituents being at least one selected from the group consisting of straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;
wherein the phenyl radical substituents, the phenylalkenyl radical substituents, the phenylalkynyl radical substituents or the biphenylalkyl radical substituents are selected from the group consisting of a straight or branched chain, saturated or unsaturated aliphatic group having 1-6 carbon atoms, halogen, nitro, carboxy, carboxy alkyl, alkoxy, hydroxy, hydroxyalkyl, perhaloalkoxy, acyl, acyloxy, acyloxyalkyl, cyano, carbalkoxy, thio, alkylhio, alkylsulfinyl, alkylsulfonyl, amino, alkylamino, dialkylamino, aminoalkyl, alkylaminoalkyl, dialkylaminoalkyl, sulfonamido, carboxamido, alkanoylamino;
W is absent or represents independently O, S, NH, NHCH₂ or N—R₅ wherein R₅ is a linear or branched alkyl group comprising 1 to 6 carbon atoms;
A is absent or represents CONH, NHCO, NHCONH;
R₄ represents H, unsubstituted or substituted alkyl from 1 to 6 carbon atoms, unsubstituted or substituted alkenyl, unsubstituted or substituted alkynyl, halogen, haloalkyl, COOH, OCH₃, NO₂, NH₂, CN, OZ′ or SZ′ where Z′ is H, or unsubstituted or substituted alkyl from 1 to 6 carbon atoms;
with the provisio that the compounds indicated below are not included

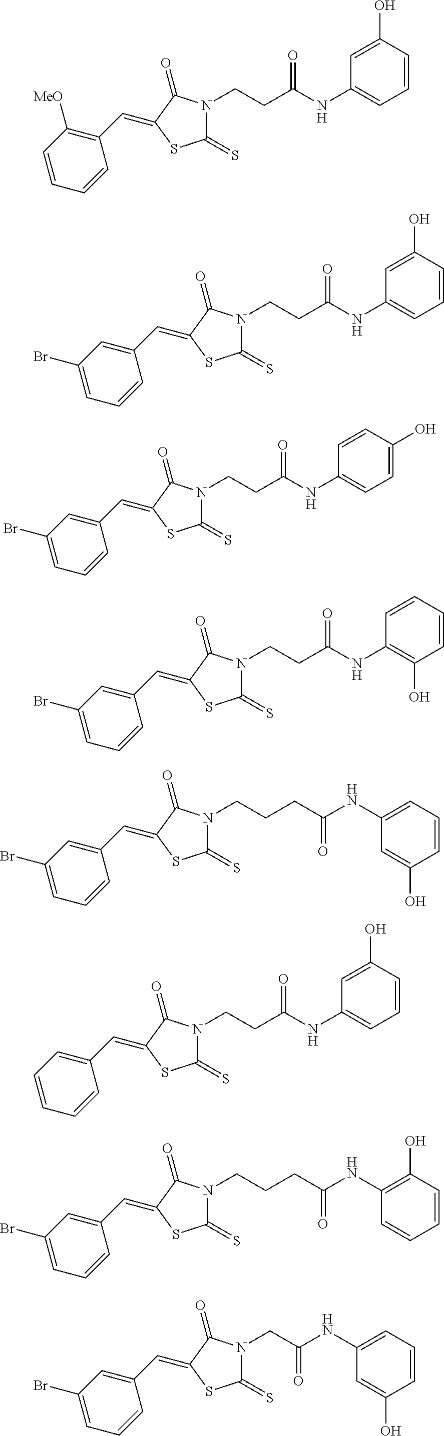

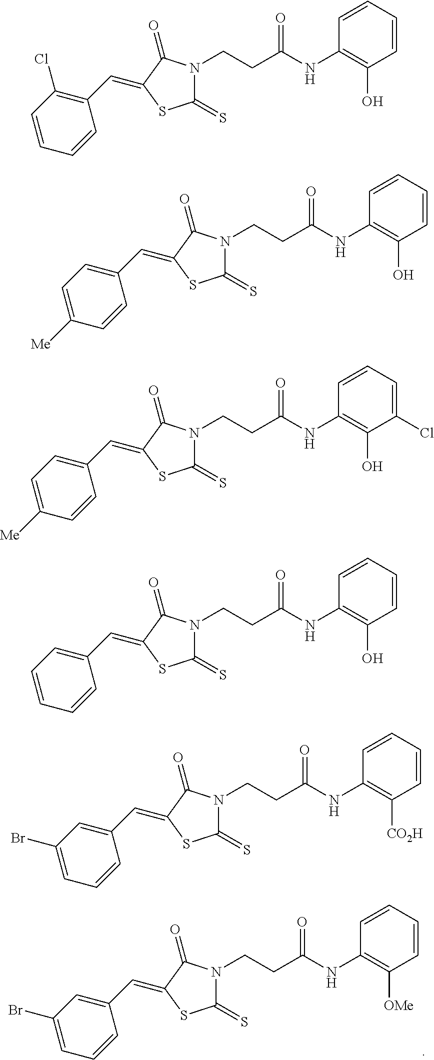

2. The compound according to claim 1 having formula 1 or 3 wherein W is absent or independently selected from the group consisting of NH, NHCH₂ or N—R₅ wherein R₅ is ethyl; R₁ is independently selected from the group consisting of ethyl, heterocyclic radical, unsubstituted or substituted phenyl radical and polycyclic radical; R₂ is selected from the group consisting of unsubstituted or substituted phenyl radical, heterocyclic radical or polycyclic radical when R₃=H or Me; alternatively, R₂ and R₃ together form a 2-oxindole.

3. The compound according to claim 1 being compound 11d, 14f, 14g, 15h, 15k, 38, FE56, FE56M, FE56F, FE56N, FE56AN or 22.

4. The compound according to claim 1 having formula 2 wherein Z=S; Y=S; X=O; n=2; 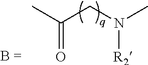
or C═O; q=0; R₂′=H; R₁ is any independently substituted phenyl radical or polycyclic radical.

5. A compound according to claim 1 being compound 35b, 35c, 35f, FE-70, FE-77, FE-69, or FE-74.

6. The compound according to claim 1 having formula 4 wherein R₂, R₃ are independently selected from the group consisting of H, unsubstituted or substituted phenyl radical; R₄=H, unsubstituted or substituted alkyl from 1 to 6 carbon atoms, COOH, OCH₃, NO₂, NH₂; A is absent or represents CONH, NHCO, and NHCONH.

7. A compound according to claim 6 being the compound EI-01, EI-01A, EI-01B, 25, 26, 27, 29, 30, or 31.

8. A compound according to claim 1 being an inhibitor of the human DEAD-box RNA helicases DDX3.

9. A compound according to claim 8 being an inhibitor of ATPase and/or helicase of the human DEAD-box RNA helicases DDX3.

10-14. (canceled)

15. A pharmaceutical composition comprising a compound of claim 1, or a pharmaceutically acceptable salt, solvate, or hydrate thereof and pharmaceutically acceptable excipients.

16. A method for inhibiting the human DEAD-box RNA helicases DDX3 comprising contacting the compound of claim 1.

17. A method for treating a viral and/or a hyperproliferative disease in a cell, comprising contacting the cell with the compound of claim 1.

18. A process for the preparation of the compound of claim 1.

19-28. (canceled)

29. A method for inhibiting the human DEAD-box RNA helicases DDX3 comprising contacting the composition of claim 15 with human DDX3, thereby inhibiting the activity of DDX3.

30. A method for treating a viral and/or a hyperproliferative disease in a cell, comprising contacting the cell with the composition of claim 15.
